(19) United States
(12) Patent Application Publication
(10) Pub. No.: US 2023/0256106 A1
(43) Pub. Date: Aug. 17, 2023

(54) DRUG DELIVERY SYSTEM FOR LOCALLY DELIVERING THERAPEUTIC AGENTS AND USES THEREOF

(71) Applicant: COVAL BIOPHARMA (SHANGHAI) CO., LTD., Shanghai (CN)

(72) Inventors: Hao ZHANG, Foster City, CA (US); Weijiang ZHANG, Redwood City, CA (US); Lichun FENG, Shanghai (CN); Guolong WU, Shanghai (CN); Dafeng LI, Shanghai (CN)

(21) Appl. No.: 18/005,356

(22) PCT Filed: Jul. 13, 2021
(86) PCT No.: PCT/CN2021/105899

§ 371 Date: Jan. 13, 2023

(30) Foreign Application Priority Data
Jul. 15, 2020 (WO) PCT/CN2020/102115

(51) Int. Cl. A61K 47/61; A61K 31/519

(52) U.S. Cl. CPC A61K 47/61; A61K 31/519

(57) ABSTRACT

Provided herein are drug delivery systems and methods for locally delivering therapeutic agents, and methods for using such drug delivery systems for the treatment of diseases.

### FIELD OF THE DISCLOSURE

The present disclosure relates to drug delivery systems and methods for locally delivering therapeutic agents, and methods for using such drug delivery systems for the treatment of diseases.

### BACKGROUND OF THE DISCLOSURE

Most of the therapeutic agents are delivered to the body systemically via oral/GI absorption or systemic injection. These delivery routes are convenient and suitable for treating systemic illnesses. However, many diseases are local disorders. Even though the therapeutical agents administered systemically can effectively treat these disorders, they may also target other tissues or binding sites that can result in side effects or adverse effects. To reduce systemic side effects, a locally administered drug delivery system is desirable. Delivering therapeutic agents to the desirable sites is not as easy as taking drugs orally or via injection. Therefore, a long term, sustained release drug delivery system for locally delivering drug is a must for such a product to be acceptable by the doctors and patients. In addition, the release profile of the therapeutic agents to maintain an effective concentration at the delivery site after the drug being administered to a subject may dramatically affect the therapeutic agents' effectiveness. Thus, drug delivery of a therapeutic agent at a specific target tissue or site within the body represents a long-time challenge in the pharmaceutic industry.

Numerous drug delivery systems have been developed to provide controlled drug delivery with tissue specificity or desired release profile. The most common local drug delivery system is to use biodegradable polymers to control the release rate of the therapeutic agents. These drug delivery systems release drugs via both biopolymer erosion and drug molecule diffusion. This complicated release control has imposed a great challenge in drug product manufacturing and quality control. Therefore, there is a continuing need for developing drug delivery system that can locally deliver therapeutic agents to specific tissues with controlled release of the therapeutic agents and reduced side effects.

There are three critical properties for a successful local drug delivery system: the ability to maintain the delivery system at the delivery site; the ability to release the therapeutic agent at a desirable rate and profile; and the ability to treat local disorder with the therapeutic agent. The present disclosure provides a different approach to fulfill these critical properties for a local drug delivery system, i.e., the biopolymers, due to their large molecular sizes, are to hold the drug delivery system at the delivery site; the therapeutic agents, are to be selected from marketed products or the activities have been proven by late stage clinical studies; and the linkers, covalently binding to the biopolymers and the therapeutic agents, are not stable chemically and upon degrading, release the therapeutic agents at a desirable rate for a specific delivery site and a specific disease.

### SUMMARY OF THE DISCLOSURE

In one aspect, the present disclosure provides a drug delivery system for locally delivering a therapeutic agent at a controlled rate, the drug delivery system comprising:

a biopolymer comprising at least a first binding group BG1 selected from the group consisting of hydroxyl group, carboxylic group, amino group, and a combination thereof;

a therapeutic agent comprising at least a second binding group BG2 selected from the group consisting of hydroxyl group, carboxylic group, amino group, amide group, amine group and a combination thereof; and

a linker covalently linking the biopolymer to the therapeutic agent and capable of retaining the therapeutic agent in the location of administration;

wherein the linker comprises a structure of formula (I):

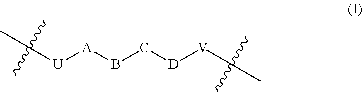

wherein

U is connected to the biopolymer through BG1 such that at least one linkage selected from ester or amide is formed, and U is selected from the group consisting of a direct bond, —N(R¹)—, —O—, —C(═O)— and

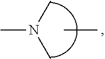

wherein

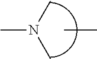

is a nitrogen-containing heterocyclyl optionally comprising one or more additional heteroatoms selected from N, O or S;

A is selected from a direct bond, alkyl and —(CH₂CH₂O)ₘ—, wherein said alkyl is optionally substituted with one or more R^{a} groups;

B is selected from the group consisting of a direct bond, alkyl, cycloalkyl, heterocyclyl, aryl, heteroaryl, —O-cycloalkyl, —O-heterocyclyl, —O-aryl, —O-heteroaryl, wherein each of alkyl, cycloalkyl, heterocyclyl, aryl and heteroaryl is optionally substituted with one or more R^{b} groups;

C is selected from a direct bond, —C(═O)—, —C(═O)N(R²)—, —N(R²)C(═O)—, —[CH₂NHC(═O)]ₙ—, —[NHC(═O)CH₂]ₙ— and —NH(CH₂)ₚC(═O)—;

D is selected from a direct bond, alkyl, and aryl, wherein said alkyl is optionally substituted with one or more R^{c} groups;

V is connected to the therapeutic agent through BG2 such that at least one linkage selected from the group consisting of amide, urea, thiourea, carbamate, thiocarbamate, phosphoramidate, aza-acetal and combination thereof is formed, and V is selected from the group consisting of a direct bond, —C(═O)—, —N(R²)C(═O)—, —N(R²)C(S)—, —OC(═O)—, —OC(═S)—, —OC(═O)OCH₂—, —C(═O)OCH₂—, —N(R²)C(═O)OCH₂—, —OP(═O)(OPh)-, and —N(R²)P(═O)(OPh)-;

R¹ and R² are independently selected from hydrogen, alkyl, alkenyl and alkynyl;

R^{a}, R^{b}, and R^{c} are independently selected from halogen, hydroxyl, amino, cyano, nitro, alkyl, alkoxyl, —C(═O)OR^{e}, and ═NH;

m is an integer from 0 to 4;

n is an integer from 1 to 4; and

p is an integer from 1 to 4.

In some embodiments, the linker in the drug delivery system provided herein comprises a structure of formula (Ia) to (Im):

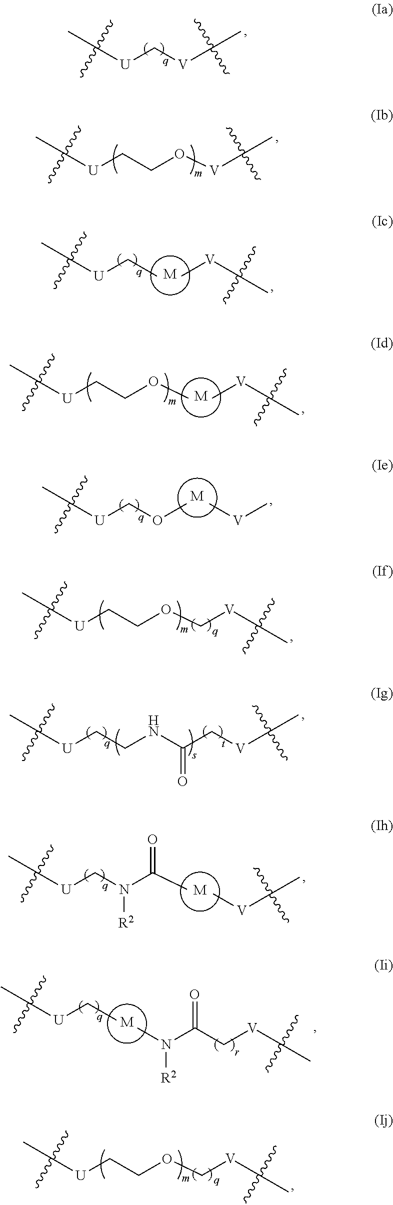

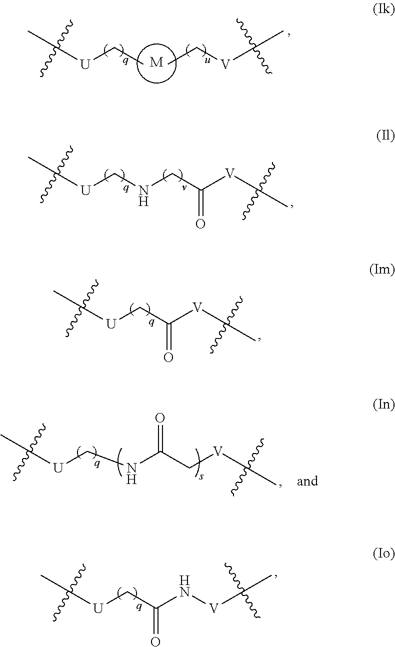

wherein,
U and V are as defined in claim 1;
M is selected from the group consisting of cycloalkyl, heterocyclyl, aryl, and heteroaryl, each of which is optionally substituted with one or more R^{b} groups; and
q, r, s, t, u and v are independently integer from 0 to 4.

In some embodiments, the biopolymer in the drug delivery system provided herein is selected from the group consisting of hyaluronic acid, chitosan, chitin, chondroitin, or derivatives thereof.

In some embodiments, the therapeutic agent in the drug delivery system provided herein is selected from the group consisting of anti-inflammatory drugs, Janus kinase (JAK) inhibitors, vascular endothelial growth factor (VEGF) inhibitors, anti-cancer drugs, and any drugs that may have severe systemic toxicities.

In a further aspect, the present disclosure provides a pharmaceutical composition comprising the drug delivery system provided herein and a pharmaceutically acceptable excipient.

In another aspect, the present disclosure provides a method of treating a disorder in a subject in need thereof, comprising administering to the subject a therapeutic effective amount of the drug delivery system or the pharmaceutical composition provided herein.

### DETAILED DESCRIPTION OF THE DISCLOSURE

Reference will now be made in detail to certain embodiments of the present disclosure, examples of which are illustrated in the accompanying structures and formulas. While the present disclosure will be described in conjunction with the enumerated embodiments, it will be understood that they are not intended to limit the present disclosure to those embodiments. On the contrary, the present disclosure is intended to cover all alternatives, modifications, and equivalents, which may be included within the scope of the present disclosure as defined by the claims. One skilled in the art will recognize many methods and materials similar or equivalent to those described herein, which could be used in the practice of the present disclosure. The present disclosure is in no way limited to the methods and materials described. In the event that one or more of the incorporated references and similar materials differs from or contradicts this application, including but not limited to defined terms, term usage, described techniques, or the like, the present disclosure controls. All references, patents, patent applications cited in the present disclosure are hereby incorporated by reference in their entireties.

It is appreciated that certain features of the present disclosure, which are, for clarity, described in the context of separate embodiments, can also be provided in combination in a single embodiment. Conversely, various features of the present disclosure, which are, for brevity, described in the context of a single embodiment, can also be provided separately or in any suitable sub-combination. It must be noted that, as used in the specification and the appended claims, the singular forms “a,” “an,” and “the” include plural forms of the same unless the context clearly dictates otherwise.

### Definitions

Definitions of specific functional groups and chemical terms are described in more detail below. For purposes of this disclosure, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, 2^{nd} Edition, University Science Books, Sausalito, 2006; Smith and March March's Advanced Organic Chemistry, 6^{th} Edition, John Wiley & Sons, Inc., New York, 2007; Larock, Comprehensive Organic Transformations, 3^{rd} Edition, VCH Publishers, Inc., New York, 2018; Carruthers, Some Modern Methods of Organic Synthesis, 4^{th} Edition, Cambridge University Press, Cambridge, 2004; the entire contents of each of which are incorporated herein by reference.

At various places in the present disclosure, linking substituents are described. It is specifically intended that each linking substituent includes both the forward and backward forms of the linking substituent. For example, —NR(CR′R″)— includes both —NR(CR′R″)— and —(CR′R″)NR—. Where the structure clearly requires a linking group, the Markush variables listed for that group are understood to be linking groups. For example, if the structure requires a linking group and the Markush group definition for that variable lists “alkyl”, then it is understood that the “alkyl” represents a linking alkylene group.

When a bond to a substituent is shown to cross a bond connecting two atoms in a ring, then such substituent may be bonded to any atom in the ring. When a substituent is listed without indicating the atom via which such substituent is bonded to the rest of the compound of a given formula, then such substituent may be bonded via any atom in such formula. Combinations of substituents and/or variables are permissible, but only if such combinations result in stable compounds.

When any variable (e.g., R¹) occurs more than one time in any constituent or formula for a compound, its definition at each occurrence is independent of its definition at every other occurrence. Thus, for example, if a group is shown to be substituted with 0-2 R¹ moieties, then the group may optionally be substituted with up to two R¹ moieties and R¹ at each occurrence is selected independently from the definition of R. Also, combinations of substituents and/or variables are permissible, but only if such combinations result in stable compounds.

As used herein, the term “Cᵢ₋ⱼ” indicates a range of the carbon atoms numbers, wherein i and j are integers and the range of the carbon atoms numbers includes the endpoints (i.e. i and j) and each integer point in between, and wherein j is greater than i. For examples, C₁₋₆ indicates a range of one to six carbon atoms, including one carbon atom, two carbon atoms, three carbon atoms, four carbon atoms, five carbon atoms and six carbon atoms. In some embodiments, the term “C₁₋₁₂” indicates 1 to 12, particularly 1 to 10, particularly 1 to 8, particularly 1 to 6, particularly 1 to 5, particularly 1 to 4, particularly 1 to 3 or particularly 1 to 2 carbon atoms.

As used herein, the term “alkyl”, whether as part of another term or used independently, refers to a saturated linear or branched-chain hydrocarbon radical, which may be optionally substituted independently with one or more substituents described below. The term “Cᵢ₋ⱼ alkyl” refers to an alkyl having i to j carbon atoms. In some embodiments, alkyl groups contain 1 to 10 carbon atoms. In some embodiments, alkyl groups contain 1 to 9 carbon atoms. In some embodiments, alkyl groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₁₀ alkyl” include, but are not limited to, methyl, ethyl, propyl, butyl, pentyl, hexyl, heptyl, octyl, nonyl, and decyl. Examples of “C₁₋₆ alkyl” are methyl, ethyl, propyl, isopropyl, n-butyl, i-butyl, s-butyl, t-butyl, n-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl, 3,3-dimethyl-2-butyl, and the like.

As used herein, the term “alkenyl”, whether as part of another term or used independently, refers to linear or branched-chain hydrocarbon radical having at least one carbon-carbon double bond, which may be optionally substituted independently with one or more substituents described herein, and includes radicals having “cis” and “trans” orientations, or alternatively, “E” and “Z” orientations. In some embodiments, alkenyl groups contain 2 to 12 carbon atoms. In some embodiments, alkenyl groups contain 2 to 11 carbon atoms. In some embodiments, alkenyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkenyl groups contain 2 carbon atoms. Examples of alkenyl group include, but are not limited to, ethylenyl (or vinyl), propenyl (allyl), butenyl, pentenyl, 1-methyl-2 buten-1-yl, 5-hexenyl, and the like.

As used herein, the term “alkynyl”, whether as part of another term or used independently, refers to a linear or branched hydrocarbon radical having at least one carbon-carbon triple bond, which may be optionally substituted independently with one or more substituents described herein. In some embodiments, alkenyl groups contain 2 to 12 carbon atoms. In some embodiments, alkynyl groups contain 2 to 11 carbon atoms. In some embodiments, alkynyl groups contain 2 to 11 carbon atoms, 2 to 10 carbon atoms, 2 to 9 carbon atoms, 2 to 8 carbon atoms, 2 to 7 carbon atoms, 2 to 6 carbon atoms, 2 to 5 carbon atoms, 2 to 4 carbon atoms, 2 to 3 carbon atoms, and in some embodiments, alkynyl groups contain 2 carbon atoms. Examples of alkynyl group include, but are not limited to, ethynyl, 1-propynyl, 2-propynyl, and the like.

As used herein, the term “alkoxyl”, whether as part of another term or used independently, refers to an alkyl group, as previously defined, attached to the parent molecule through an oxygen atom. The term “Cᵢ₋ⱼ alkoxy” means that the alkyl moiety of the alkoxy group has i to j carbon atoms. In some embodiments, alkoxy groups contain 1 to 10 carbon atoms. In some embodiments, alkoxy groups contain 1 to 9 carbon atoms. In some embodiments, alkoxy groups contain 1 to 8 carbon atoms, 1 to 7 carbon atoms, 1 to 6 carbon atoms, 1 to 5 carbon atoms, 1 to 4 carbon atoms, 1 to 3 carbon atoms, or 1 to 2 carbon atoms. Examples of “C₁₋₆ alkoxyl” include, but are not limited to, methoxy, ethoxy, propoxy (e.g. n-propoxy and isopropoxy), t-butoxy, neopentoxy, n-hexoxy, and the like.

As used herein, the term “amide” refers to —C(═O)NR′—, wherein R′ represents hydrogen, an N-protecting group, alkyl, alkenyl, alkynyl, alkoxy, cycloalkyl, heterocyclyl, aryl, heteroaryl and other suitable organic groups.

As used herein, the term “amine” refers to derivatives of ammonia, wherein one or more hydrogen atoms are replaced by a substituent, and can be represented by N(H)ₙ(R′)₃₋ₙ wherein n is 0, 1, or 2, and each R′ is independently hydroxyl, nitro, an N-protecting group, alkyl, alkenyl, alkynyl, alkoxy, cycloalkyl, heterocyclyl, aryl, heteroaryl and other suitable organic groups, or two R′ together with the nitrogen atom to which they are attached form an optionally substituted heterocyclyl or heteroaryl.

As used herein, the term “amino” refers to —NH₂.

As used herein, the term “aryl”, whether as part of another term or used independently, refers to monocyclic and polycyclic ring systems having a total of 5 to 20 ring members, wherein at least one ring in the system is aromatic and wherein each ring in the system contains 3 to 12 ring members. Examples of “aryl” include, but are not limited to, phenyl, biphenyl, naphthyl, anthracyl and the like, which may bear one or more substituents. Also included within the scope of the term “aryl”, as it is used herein, is a group in which an aromatic ring is fused to one or more additional rings. In the case of polycyclic ring system, only one of the rings needs to be aromatic (e.g., 2,3-dihydroindole), although all of the rings may be aromatic (e.g., quinoline). The second ring can also be fused or bridged. Examples of polycyclic aryl include, but are not limited to, benzofuranyl, indanyl, phthalimidyl, naphthimidyl, phenanthridinyl, or tetrahydronaphthyl, and the like. Aryl groups can be substituted at one or more ring positions with substituents as described above.

As used herein, the term “aza-acetal” refers to —N—CH₂—O—.

As used herein, the term “carboxylic group” or “carboxyl” refers to —COOH.

As used herein, the term “cycloalkyl”, whether as part of another term or used independently, refer to a monovalent non-aromatic, saturated or partially unsaturated monocyclic and polycyclic ring system, in which all the ring atoms are carbon and which contains at least three ring forming carbon atoms. In some embodiments, the cycloalkyl may contain 3 to 12 ring forming carbon atoms, 3 to 10 ring forming carbon atoms, 3 to 9 ring forming carbon atoms, 3 to 8 ring forming carbon atoms, 3 to 7 ring forming carbon atoms, 3 to 6 ring forming carbon atoms, 3 to 5 ring forming carbon atoms, 4 to 12 ring forming carbon atoms, 4 to 10 ring forming carbon atoms, 4 to 9 ring forming carbon atoms, 4 to 8 ring forming carbon atoms, 4 to 7 ring forming carbon atoms, 4 to 6 ring forming carbon atoms, 4 to 5 ring forming carbon atoms. Cycloalkyl groups may be saturated or partially unsaturated. Cycloalkyl groups may be substituted. In some embodiments, the cycloalkyl group may be a saturated cyclic alkyl group. In some embodiments, the cycloalkyl group may be a partially unsaturated cyclic alkyl group that contains at least one double bond or triple bond in its ring system. In some embodiments, the cycloalkyl group may be monocyclic or polycyclic. Examples of monocyclic cycloalkyl group include, but are not limited to, cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl and cyclododecyl. Examples of polycyclic cycloalkyl group include, but are not limited to, adamantyl, norbornyl, fluorenyl, spiro-pentadienyl, spiro[3.6]-decanyl, bicyclo[1,1,1]pentenyl, bicyclo[2,2,1]heptenyl, and the like.

As used herein, the term “cyano” refers to —CN.

As used herein, the term “ester” refers to —C(═O)O—.

As used herein, the term “carbamate” refers to —NR′(C═O)O—, wherein R′ represents hydrogen, an N-protecting group, alkyl, alkenyl, alkynyl, alkoxy, cycloalkyl, heterocyclyl, aryl, heteroaryl and other suitable organic groups.

As used herein, the term “thiocarbamate” refers to —NR′(C═S)O—, wherein R′ represents hydrogen, an N-protecting group, alkyl, alkenyl, alkynyl, alkoxy, cycloalkyl, heterocyclyl, aryl, heteroaryl and other suitable organic groups.

As used herein, the term “halogen” refers to an atom selected from fluorine (or fluoro), chlorine (or chloro), bromine (or bromo) and iodine (or iodo).

As used herein, the term “heteroatom” refers to nitrogen, oxygen, or sulfur, and includes any oxidized form of nitrogen or sulfur, and any quaternized form of a basic nitrogen (including N-oxides).

As used herein, the term “heteroaryl”, whether as part of another term or used independently, refers to an aryl group having, in addition to carbon atoms, one or more heteroatoms. The heteroaryl group can be monocyclic. Examples of monocyclic heteroaryl include, but are not limited to, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, benzofuranyl and pteridinyl. The heteroaryl group also includes polycyclic groups in which a heteroaromatic ring is fused to one or more aryl, cycloaliphatic, or heterocyclyl rings, where the radical or point of attachment is on the heteroaromatic ring. Examples of polycyclic heteroaryl include, but are not limited to, indolyl, isoindolyl, benzothienyl, benzofuranyl, benzo[1,3]dioxolyl, dibenzofuranyl, indazolyl, benzimidazolyl, benzthiazolyl, quinolyl, isoquinolyl, dihydroquinolinyl, dihydroisoquinolinyl, tetrahydroquinolinyl, tetrahydroisoquinolinyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and the like.

As used herein, the term “heterocyclyl” refers to a saturated or partially unsaturated carbocyclyl group in which one or more ring atoms are heteroatoms independently selected from oxygen, sulfur, nitrogen, phosphorus, and the like, the remaining ring atoms being carbon, wherein one or more ring atoms may be optionally substituted independently with one or more substituents. In some embodiments, the heterocyclyl is a saturated heterocyclyl. In some embodiments, the heterocyclyl is a partially unsaturated heterocyclyl having one or more double bonds in its ring system. In some embodiments, the heterocyclyl may contains any oxidized form of carbon, nitrogen or sulfur, and any quaternized form of a basic nitrogen. “Heterocyclyl” also includes radicals wherein the heterocyclyl radicals are fused with a saturated, partially unsaturated, or fully unsaturated (i.e., aromatic) carbocyclic or heterocyclic ring. The heterocyclyl radical may be carbon linked or nitrogen linked where such is possible. In some embodiments, the heterocycle is carbon linked. In some embodiments, the heterocycle is nitrogen linked. For example, a group derived from pyrrole may be pyrrol-1-yl (nitrogen linked) or pyrrol-3-yl (carbon linked). Further, a group derived from imidazole may be imidazol-1-yl (nitrogen linked) or imidazol-3-yl (carbon linked).

In some embodiments, the term “3- to 12-membered heterocyclyl” refers to a 3- to 12-membered saturated or partially unsaturated monocyclic or polycyclic heterocyclic ring system having 1 to 3 heteroatoms independently selected from nitrogen, oxygen, or sulfur. The fused, spiro and bridged ring systems are also included within the scope of this definition. Examples of monocyclic heterocyclyl include, but are not limited to oxetanyl, 1,1-dioxothietanylpyrrolidyl, tetrahydrofuryl, tetrahydrothienyl, pyrrolyl, furanyl, thienyl, pyrazolyl, imidazolyl, triazolyl, oxazolyl, thiazolyl, piperidyl, piperazinyl, piperidinyl, morpholinyl, pyridinyl, pyrazinyl, pyrimidinyl, pyridazinyl, triazinyl, pyridonyl, pyrimidonyl, pyrazinonyl, pyrimidonyl, pyridazonyl, pyrrolidinyl, triazinonyl, and the like. Examples of fused heterocyclyl include, but are not limited to, phenyl fused ring or pyridinyl fused ring, such as quinolinyl, isoquinolinyl, tetrahydroquinolinyl, tetrahydroisoquinolinyl, quinoxalinyl, quinolizinyl, quinazolinyl, azaindolizinyl, pteridinyl, chromenyl, isochromenyl, indolyl, isoindolyl, indolizinyl, indazolyl, purinyl, benzofuranyl, isobenzofuranyl, benzimidazolyl, benzothienyl, benzothiazolyl, carbazolyl, phenazinyl, phenothiazinyl, phenanthridinyl, imidazo[1,2-a]pyridinyl, [1,2,4]triazolo[4,3-a]pyridinyl, [1,2,3]triazolo[4,3-a]pyridinyl groups, and the like. Examples of spiro heterocyclyl include, but are not limited to, spiropyranyl, spirooxazinyl, and the like. Examples of bridged heterocyclyl include, but are not limited to, morphanyl, hexamethylenetetraminyl, 3-aza-bicyclo[3.1.0]hexane, 8-aza-bicyclo[3.2.1]octane, 1-aza-bicyclo[2.2.2]octane, 1,4-diazabicyclo[2.2.2]octane (DABCO), and the like.

As used herein, the term “hydroxyl” refers to —OH.

As used herein, the term “nitro” refers to —NO₂.

As used herein, the term “urea” refers to —NR′(C═O)NR″—, wherein R′ and R″ each independently represents hydrogen, an N-protecting group, alkyl, alkenyl, alkynyl, alkoxy, cycloalkyl, heterocyclyl, aryl, heteroaryl and other suitable organic groups.

As used herein, the term “thiourea” refers to —NR′H(C═S)NR″—, wherein R′ and R″ each independently represents hydrogen, an N-protecting group, alkyl, alkenyl, alkynyl, alkoxy, cycloalkyl, heterocyclyl, aryl, heteroaryl and other suitable organic groups.

As used herein, the term “phosphoramidate” refers to —(NR′)P(═O)(OR′)ₐ(NR″)_{b}—, wherein R′ and R″ are independently null, hydrogen, alkyl, alkenyl, alkynyl, alkoxy, cycloalkyl, heterocyclyl, aryl, heteroaryl and other suitable organic groups, a and b are independently 0, 1 or 2.

As used herein, the term “binding group” or “BG” refers to a group at a particular position within a first entity (e.g., biopolymer, therapeutic agent as provided herein), which is capable of reacting with another group from a second entity (e.g., linker as provided herein) to form a linkage, thereby joining the two entities together to form one entity. For example, carboxyl groups included in one entity may react with amino groups included in another entity to form amide linkage that links the two entities together, wherein the carboxyl and amino groups can be regarded as binding groups.

As used herein, the term “linkage” or “linker” refers to bonds or chemical moiety formed from a chemical reaction between the functional groups of at least two entities to be linked, thereby forming one molecule or maintaining association of the entities in sufficiently close proximity. A linker can be integrated in the resulting linked molecule or structure, with or without its reacted functional groups. Such linkages may be covalent or non-covalent. Hydrolytically unstable or degradable linkages mean that the linkages are degradable in water or in aqueous solutions, including for example, body fluid such as blood. Enzymatically unstable or degradable linkages mean that the linkage can be degraded by one or more enzymes. Such degradable linkages include, but are not limited to ester linkages formed by the carboxylic acid in one entity with alcohol groups on a biologically active agent, wherein such ester groups generally hydrolyze under physiological conditions to release the biologically active agent. Other hydrolytically degradable linkages include but are not limited to carbonate linkages, imine linkages resulted from reaction of an amine and an aldehyde, phosphate ester linkages resulted from reaction of a phosphate group and an alcohol, hydrazone linkages resulted from reaction of a hydrazide and an aldehyde, acetal linkages resulted from reaction of an aldehyde and an alcohol, amide linkages resulted from reaction of an amine group and a carboxyl group.

As used herein, the term “partially unsaturated” refers to a radical that includes at least one double or triple bond. The term “partially unsaturated” is intended to encompass rings having multiple sites of unsaturation, but is not intended to include aromatic (i.e., fully unsaturated) moieties.

As used herein, the term “pharmaceutically acceptable” indicates that the substance or composition is compatible chemically and/or toxicologically, with the other ingredients comprising a formulation, and/or the subjects being treated therewith.

As used herein, the term “substituted”, whether preceded by the term “optionally” or not, means that one or more hydrogens of the designated moiety are replaced with a suitable substituent. It will be understood that “substitution” or “substituted with” includes the implicit proviso that such substitution is in accordance with permitted valence of the substituted atom and that the substitution results in a stable or chemically feasible compound, e.g., which does not spontaneously undergo transformation such as by rearrangement, cyclization, elimination, etc. Unless otherwise indicated, an “optionally substituted” group may have a suitable substituent at each substitutable position of the group, and when more than one position in any given structure may be substituted with more than one substituent selected from a specified group, the substituent may be either the same or different at every position. It will be understood by those skilled in the art that substituents can themselves be substituted, if appropriate. Unless specifically stated as “unsubstituted”, references to chemical moieties herein are understood to include substituted variants. For example, reference to an “aryl” group or moiety implicitly includes both substituted and unsubstituted variants.

As used herein, the terms “therapeutic agent”, “drug”, “biologically active molecule”, “biologically active agent”, “active agent” and the like refer to any substance which can affect any physical or biochemical properties of a biological organism, including but not limited to viruses, bacteria, fungi, plants, animals, and human. In particular, as used herein, therapeutic agents include any substance intended for diagnosis, cure, mitigation, treatment, or prevention of disease in humans or other animals, or to otherwise enhance physical or mental well-being of humans or animals.

Drug delivery of therapeutic agents to specific tissues or sites within a body presents a variety of challenges, particularly where local delivery of a high dose of a therapeutic agent having poor aqueous solubility to a specific tissue is desired, and where avoidance of high systemic concentration of the therapeutic agent leading to toxic side effects is desired.

Therefore, the present disclosure in one aspect provides a drug delivery system capable of locally delivering a therapeutic agent at a controlled rate. In some embodiments, the drug delivery system comprises a biopolymer, a therapeutic agent and a linker covalently linking the biopolymer to the therapeutic agent and capable of retaining the therapeutic agent in the location of administration.

### Biopolymer

Biopolymers are natural polymers produced by living organisms, and contain monomeric units that are covalently bonded to form larger structures. There are three main classes of biopolymers, classified according to the monomeric units used and the structure of the biopolymer formed: polynucleotides, polypeptides, and polysaccharides. More specifically, polynucleotides, such as RNA and DNA, are long polymers composed of 13 or more nucleotide monomers. Polypeptides or proteins, are short polymers of amino acids and some major examples include collagen, actin, and fibrin. Polysaccharides, are often linear bonded polymeric carbohydrate structures and some examples include cellulose and alginate. Other examples of biopolymers include rubber, suberin, melanin and lignin.

A variety of biopolymers are useful as polymeric delivery vehicles for delivering the therapeutic agents to target cells or tissues. Biopolymers suitable for a particular application are selected based on their ability to target particular tissues, organs or cells, and their in vivo stability, i.e., the in vivo residence time in the circulatory system, or specific tissues, cells or organs.

In some embodiments, the biopolymer is selected from biocompatible polymers comprising at least a first binding group BG1, which is capable of reacting with a reactive functional group from a second entity (e.g., linker as provided herein) to form a linkage, thereby linking a biopolymer to the second entity (e.g., the linker). The term “biocompatible”, as used herein, refers to a substance that has no medically unacceptable toxic or injurious effects on biological function, or which is tolerated by the body.

In some embodiments, the biopolymer is selected from biocompatible polymers comprising at least a first binding group BG1, wherein BG1 is selected from the group consisting of hydroxyl group, carboxylic group, amino group, and a combination thereof. The BG1 serves as binding sites for the conjugation of linkers suitable for linking the therapeutic agents to the biopolymer. The BG1 may be present at any site within the backbone of the biopolymer, and thus the linkages formed between the biopolymer and the linker may be present at any part of the biopolymer.

In some embodiments, the BG1 for reacting with the reactive functional group from the linker can be the same or different. In certain embodiments, the BG1 of the biopolymer is the same. In certain embodiments, the BG1 of the biopolymer is different.

In some embodiments, the biopolymers are biocompatible polymers comprising carboxylic group as BG1, which is capable of reacting with a reactive functional group of a suitable linker to form a linkage connecting the carboxylic group-containing biopolymer to the linker.

In certain embodiments, the reactive functional group of the linker is amino or amine, which reacts with the carboxylic group of the biopolymer such that an amide linkage is formed.

In certain embodiments, the reactive functional group of the linker is hydroxy, which reacts with the carboxylic group of the biopolymer such that an ester linkage is formed.

In certain embodiments, the reactive functional group of the linker is halogen, which reacts with the carboxylic group of the biopolymer such that an ester linkage is formed.

In some embodiments, the biopolymers are biocompatible polymers comprising amino group as BG1, which is capable of reacting with a reactive functional group of a suitable linker to form a linkage, thereby producing a biopolymer-linker conjugate.

In certain embodiments, the reactive functional group of the linker is a carboxylic group, which reacts with the hydroxy group of the biopolymer such that an ester linkage is formed.

In some embodiments, the linkage formed from the reaction between the BG1 of the biopolymer and the reactive functional group of the linker is selected from the group consisting of —C(O)N(R¹)—,

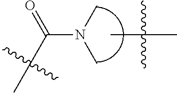

and —C(O)O—, wherein R¹ is selected from the group consisting of hydrogen, alkyl, alkenyl and alkynyl,

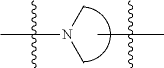

is a nitrogen-containing heterocyclyl optionally comprising one or more additional heteroatoms selected from N, O or S.

In certain embodiments, R¹ is hydrogen.

In certain embodiments,

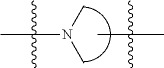

selected from the group consisting of:

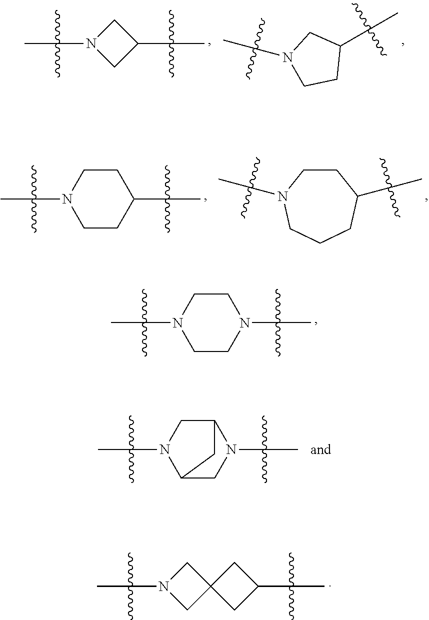

In some embodiments, the biopolymer may be selected from the group consisting of hyaluronic acid (HA), dextran, cellulose, amylose, chitosan, chitin, chondroitin, gelatin, alginate, carrageenan, gellan, guar gum, pectin, scleroglucan, and xanthan.

In some embodiments, the biopolymer may have a number average molecular weight ranging from 400 to 3,000,000 Da, for example, from 1,000 to 3,000,000 Da, from 5,000 to 3,000,000 Da, from 10,000 to 3,000,000 Da, from 20,000 to 3,000,000 Da, from 30,000 to 3,000,000 Da, from 40,000 to 3,000,000 Da, from 50,000 to 3,000,000 Da, or from 50,000 to 2,000,000 Da.

In certain embodiments, the biopolymer may be selected from the group consisting of HA, chitosan, chitin, chondroitin, or derivatives thereof.

In certain embodiments, the biopolymer is HA. In certain embodiments, the HA can derive from any source.

In certain embodiments, the HA may have a number average molecular weight ranging from 400 to 3,000,000 Da, for example, from 1,000 to 3,000,000 Da, from 5,000 to 3,000,000 Da, from 10,000 to 3,000,000 Da, from 20,000 to 3,000,000 Da, from 30,000 to 3,000,000 Da, from 40,000 to 3,000,000 Da, from 50,000 to 3,000,000 Da, or from 50,000 to 2,000,000 Da.

### Therapeutic Agent

The present disclosure provides improved delivery system for local delivery of a variety of therapeutic agent.

In some embodiments, the therapeutic agent comprises at least a second binding group BG2, which is capable of reacting with a reactive functional group from a second entity (e.g., linker as provided herein) and an optional co-reactant to form a linkage, thereby linking the therapeutic agent to the second entity (e.g., the linker).

In some embodiments, the therapeutic agent comprises at least a second binding group BG2 selected from the group consisting of hydroxyl group, carboxylic group, amino group, amide group, amine group and a combination thereof. The BG2 serves as a binding site for the conjugation of linkers suitable for linking the therapeutic agents to the biopolymer.

In some embodiments, the therapeutic agent comprises amine group as BG2, which is capable of reacting with a reactive functional group of a suitable linker and an optional co-reactant to form a linkage connecting the amine-containing therapeutic agent to the linker.

In certain embodiments, the amine group in the therapeutic agent reacts with the reactive functional group of a linker and an optional co-reactant such that the therapeutic agent is linked to the linker via a direct bond, an amide linkage, a urea linkage, a thiourea linkage, a carbamate linkage, a thiocarbamate linkage, an aza-acetal linkage, a phosphoramidate linkage, and the like.

In certain embodiments, the linkage formed from the reaction involving the BG2 of the therapeutic agent and the reactive functional group of a linker and an optional co-reactant is selected from the group consisting of —N(R′)₂—, —C(═O)N(R′)—, —C(═O)N(R′)₂—, —N(R²)C(═O)N(R′)₂—, —N(R²)C(═S)N(R′)₂—, —OC(═O)N(R′)₂—, —OC(═S)N(R′)₂—, —OC(═O)OCH₂N(R′)₂—, —N(R²)C(═O)OCH₂N(R′)₂—, —OP(═O)(OPh)N(R′)₂—, and —N(R²)P(═O)(OPh)N(R′)₂—, wherein R² is selected from the group consisting of hydrogen, alkyl, alkenyl and alkynyl, and R′ is independently selected from the group consisting of hydrogen, alkyl, alkenyl and alkynyl, or two R′ together with the nitrogen atom to which they both attached form a heterocyclyl.

In certain embodiments, R² is hydrogen.

In some embodiments, the therapeutic agent comprises carboxylic group as BG2, which is capable of reacting with a reactive functional group of a suitable linker and an optional co-reactant to form a linkage connecting the carboxylic group-containing therapeutic agent to the linker.

In certain embodiments, the carboxylic group in the therapeutic agent reacts with the reactive functional group of a linker and an optional co-reactant such that the therapeutic agent is linked to the linker via an ester linkage.

In some embodiments, the therapeutic agent comprises hydroxyl group as BG2, which is capable of reacting with a reactive functional group of a suitable linker and an optional co-reactant to form a linkage connecting the hydroxyl-containing therapeutic agent to the linker.

In certain embodiments, the hydroxyl group in the therapeutic agent reacts with the reactive functional group of a linker and an optional co-reactant such that the therapeutic agent is linked to the linker via an ester linkage.

In some embodiments, the therapeutic agent to be delivered is selected from the group consisting of nonsteroidal anti-inflammatory drugs (NSAIDs), Janus kinase (JAK) inhibitors, vascular endothelial growth factor (VEGF) inhibitors, anti-cancer drugs, and any drugs that may have severe systemic toxicities.

In some embodiments, the therapeutic agent to be delivered is NSAID selected from the group consisting of Piroxicam, Meloxicam, and Diclofenac.

In some embodiments, the therapeutic agent to be delivered is JAK inhibitor selected from the group consisting of Tofacitinib, Ruxolitinib, Baricitinib, Peficitinib, Fedratinib, Oclacitinib and Upadacitinib.

In some embodiments, the therapeutic agent to be delivered is VEGF inhibitor selected from the group consisting of Axitinib, Lapatinib, Lenvatinib, Pazopanib, Nintedanib, Sunitinib, and Vandetanib.

In some embodiments, the therapeutic agent to be delivered is Tofacitinib.

In some embodiments, the therapeutic agent to be delivered is Upadacitinib.

In some embodiments, the therapeutic agent to be delivered is Ruxolitinib.

In some embodiments, the therapeutic agent to be delivered is Baricitinib.

In some embodiments, the therapeutic agent to be delivered is Oclacitinib.

In some embodiments, the therapeutic agent to be delivered is Nintedanib.

In some embodiments, the therapeutic agent to be delivered is Sunitinib.

### Linker

The improved local delivery of therapeutic agents is achieved by linking a therapeutic agent to a biopolymer via a suitable linker. By selecting suitable linkers, the releasing rate of the therapeutic agent from the biopolymer can be controlled, thereby providing improved delivery of the therapeutic agent to target cells or tissues.

In some embodiments, a plurality of the linkers can be attached to a therapeutic agent via a cleavable linkage which is cleaved under biological conditions, thereby releasing the therapeutic agent.

A “cleavable linkage” is a relatively labile bond that cleaves under physiological conditions. An exemplary releasable linkage is a hydrolyzable bond that cleaves upon reaction with water (i.e., is hydrolyzed). The tendency of a bond to hydrolyze in water may depend not only on the general type of linkage connecting two atoms but also on the substituents attached to these atoms. Appropriate hydrolytically unstable or weak linkages include but are not limited to carboxylate ester, phosphate ester, anhydrides, acetals, ketals, acyloxyalkyl ether, imines, orthoesters, peptides, oligonucleotides, thioesters, urea, thiourea, carbamate, thiocabamate, phosphoramidate and carbonates. Certain functional groups have atoms that may be chemically degraded by a process other than hydrolysis. Exemplary releaseable linkages in this category include certain carbamates and Fmoc derivatives. Certain molecules containing these kinds of functionalities appropriately bonded may undergo chemical degradation (release) upon action of a base. In such cases “cleave” may occur at higher values of pH or through the action of biological molecules that contain basic moieties (e.g. histidines). Another exemplary cleavable linkage is an enzymatically cleavable linkage. An “enzymatically cleavable linkage” means a linkage that is subject to cleavage by one or more enzymes.

In some embodiments, the linker is attached to the biopolymer via a linkage formed from a reactive functional group of the linker and the BG1 in the biopolymer, and is attached to the therapeutic agent via a linkage formed from another reactive functional group of the linker and the BG2 in the therapeutic agent.

In some embodiments, the linker comprises a structure of formula (I):

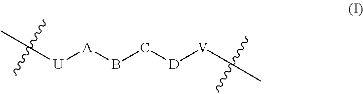

wherein

U is connected to the biopolymer through BG1 of the biopolymer such that at least one linkage selected from ester or amide is formed, and U is selected from the group consisting of a direct bond, —N(R¹)—, —O—, —C(═O)— and

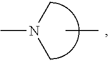

wherein

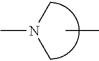

is a nitrogen-containing heterocyclyl optionally comprising one or more additional heteroatoms selected from N, O or S;

A is selected from a direct bond, alkyl and —(CH₂CH₂O)ₘ—, wherein said alkyl is optionally substituted with one or more R^{a} groups;

B is selected from the group consisting of a direct bond, alkyl, cycloalkyl, heterocyclyl, aryl, heteroaryl, —O-cycloalkyl, —O-heterocyclyl, —O-aryl, —O-heteroaryl, wherein each of alkyl, cycloalkyl, heterocyclyl, aryl and heteroaryl is optionally substituted with one or more R^{b} groups;

C is selected from a direct bond, —C(═O)—, —C(═O)N(R²)—, —N(R²)C(═O)—, —[CH₂NHC(═O)]ₙ—, —[NHC(═O)CH₂]ₙ—, and —NH(CH₂)ₚC(═O)—;

D is selected from a direct bond, alkyl, and aryl, wherein said alkyl is optionally substituted with one or more R^{c} groups;

V is connected to the therapeutic agent through BG2 in the therapeutic agent such that at least one linkage selected from the group consisting of amide, urea, thiourea, carbamate, thiocarbamate, phosphoramidate, aza-acetal, and combination thereof is formed, and V is selected from the group consisting of a direct bond, —C(═O)—, —N(R²)C(═O)—, —N(R²)C(S)—, —OC(═O)—, —OC(═S)—, —OC(═O)OCH₂—, —C(═O)OCH₂—, —N(R²)C(═O)OCH₂—, —OP(═O)(OPh)-, and —N(R²)P(═O)(OPh)-;

R¹ and R² are independently selected from the group consisting of hydrogen, alkyl, alkenyl and alkynyl;

R^{a}, R^{b}, and R^{c} are independently selected from halogen, hydroxyl, amino, cyano, nitro, alkyl, alkoxyl, —C(═O)OR^{e}, and ═NH;

R^{e} is an alkyl;

m is an integer from 0 to 4;

n is an integer from 1 to 4; and

p is an integer from 1 to 4.

In some embodiments, BG1 is a carboxylic group and U is —N(R¹)—, such that an amide linkage is formed to attach the biopolymer to the linker.

In some embodiments, BG1 is a carboxylic group and U is

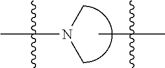

such that an amide linkage is formed to attach the biopolymer to the linker.

In certain embodiments, BG1 is a carboxylic group and U is

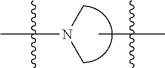

selected from the group consisting of:

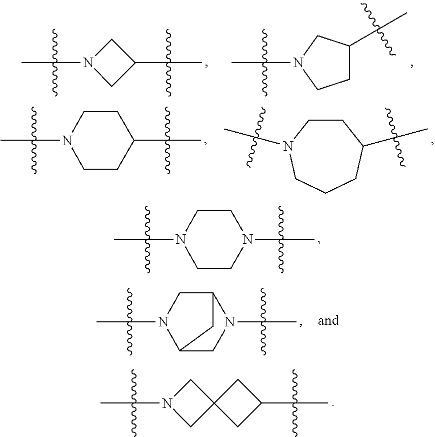

In some embodiments, BG1 is a hydroxyl group and U is —C(═O)—, such that an ester linkage is formed to attach the biopolymer to the linker.

In some embodiments, BG1 is a carboxylic group and U is —O— or a direct bond, such that an ester linkage is formed to attach the biopolymer to the linker.

In some embodiments, BG1 is an amino group and U is —C(═O)—, such that an amide linkage is formed to attach the biopolymer to the linker.

In some embodiments, BG2 is an amine group, and V is selected from one of the following:
(a) a direct bond;
(b) —N(R²)C(═O)— which is connected to the therapeutic agent through BG2 such that a urea linkage is formed to attach the therapeutic agent to the linker;
(c) —N(R²)C(S)— which is connected to the therapeutic agent through BG2 such that a thiourea linkage is formed to attach the therapeutic agent to the linker;
(d) —OC(═O)— which is connected to the therapeutic agent through BG2 such that a carbamate linkage is formed to attach the therapeutic agent to the linker;
(e) —OC(═S)— which is connected to the therapeutic agent through BG2 such that a thiocarbamate linkage is formed to attach the therapeutic agent to the linker;
(f) —OC(═O)OCH₂— which is connected to the therapeutic agent through BG2 such that an aza-acetal linkage is formed to attach the therapeutic agent to the linker;
(g) —C(═O)OCH₂— which is connected to the therapeutic agent through BG2 such that an aza-acetal linkage is formed;
(h) —N(R²)C(═O)OCH₂— which is connected to the therapeutic agent through BG2 such that an aza-acetal linkage is formed to attach the therapeutic agent to the linker;
(i) —OP(═O)(OPh)- which is connected to the therapeutic agent through BG2 such that a phosphoramidate linkage is formed to attach the therapeutic agent to the linker; or
(j) —N(R²)P(═O)(OPh)- which is connected to the therapeutic agent through BG2 such that a phosphoramidate linkage is formed to attach the therapeutic agent to the linker;
(k) —C(═O)— which is connected to the therapeutic agent through BG2 such that an amide linkage is formed.

In certain embodiments, BG2 is a carboxylic group and V is —O— or a direct bond, such that an ester linkage is formed to attach the therapeutic agent to the linker.

In certain embodiments, BG2 is a hydroxyl group and V is —C(═O)—, such that an ester linkage is formed to attach the therapeutic agent to the linker.

In some embodiments, A is a direct bond.

In some embodiments, A is alkyl optionally substituted with one or more R^{a} groups. In certain embodiments, A is C₁₋₁₀ alkyl optionally substituted with one or more R^{a} groups. In certain embodiments, A is C₁₋₈ alkyl optionally substituted with one or more R^{a} groups.

In some embodiments, R^{a} is —C(═O)OR^{e}, wherein R^{e} is alkyl. In certain embodiments, R^{a} is —C(═O)OR^{e}, wherein R^{e} is C₁₋₈ alkyl, C₁₋₇ alkyl, C₁₋₆ alkyl, C₁₋₅ alkyl, C₁₋₄ alkyl, or C₁₋₃ alkyl. In certain embodiments, R^{a} is —C(═O)OCH₃.

In some embodiments, A is —(CH₂CH₂O)ₘ—.

In certain embodiments, m is an integer from 0 to 4. In certain embodiments, m is 1. In certain embodiments, m is 2. In certain embodiments, m is 3. In certain embodiments, m is 4.

In some embodiments, B is a direct bond.

In some embodiments, B is an alkyl. In certain embodiments, B is C₁₋₆ alkyl, C₁₋₅ alkyl, C₁₋₄ alkyl, C₁₋₃ alkyl, or C₁₋₂ alkyl. In certain embodiments, B is ethyl.

In some embodiments, B is cycloalkyl, aryl or heteroaryl.

In some embodiments, B is cycloalkyl.

In certain embodiment, B is saturated cycloalkyl. In certain embodiment, B is partially unsaturated cycloalkyl.

In certain embodiments, B is 3 to 8 membered cycloalkyl, 3 to 7 membered cycloalkyl, 3 to 6 membered cycloalkyl, 3 to 5 membered cycloalkyl, or 3 to 4 membered cycloalkyl.

In certain embodiments, B is saturated 3 to 6 membered cycloalkyl. In certain embodiments, B is a cyclohexyl.

In some embodiments, B is aryl. In certain embodiments, B is 5 to 12 membered aryl, 5 to 10 membered aryl, 5 to 8 membered aryl, or 5 to 6 membered aryl.

In certain embodiments, B is a phenyl.

In some embodiments, B is heteroaryl. In certain embodiments, B is 5 to 12 membered heteroaryl, 5 to 10 membered heteroaryl, 5 to 8 membered heteroaryl, or 5 to 6 membered heteroaryl.

In certain embodiments, B is pyridinyl.

In some embodiments, B is —O-aryl. In certain embodiments, B is —O— phenyl.

In some embodiments, A is a direct bond, and B is selected from the group consisting of a direct bond, cycloalkyl, heterocyclyl, aryl, and heteroaryl.

In certain embodiments, A is a direct bond, and B is selected from the group consisting of a direct bond, cycloalkyl, aryl, and heteroaryl.

In certain embodiments, A is a direct bond, and B is a direct bond.

In certain embodiments, A is a direct bond, and B is 3 to 8 membered cycloalkyl, 3 to 7 membered cycloalkyl, 3 to 6 membered cycloalkyl, 3 to 5 membered cycloalkyl, or 3 to 4 membered cycloalkyl. In certain embodiments, A is a direct bond, and B is a cyclohexyl.

In certain embodiments, A is a direct bond, and B is 5 to 12 membered aryl, 5 to 10 membered aryl, 5 to 8 membered aryl, or 5 to 6 membered aryl. In certain embodiments, A is a direct bond, and B is phenyl.

In certain embodiments, A is a direct bond, and B is 5 to 12 membered heteroaryl, 5 to 10 membered heteroaryl, 5 to 8 membered heteroaryl, or 5 to 6 membered heteroaryl. In certain embodiments, A is a direct bond, and B is pyridyl.

In some embodiments, A is optionally substituted alkyl, and B is selected from the group consisting of a direct bond, cycloalkyl, heterocyclyl, aryl, heteroaryl, —O-cycloalkyl, —O-heterocyclyl, —O-aryl, and —O-heteroaryl.

In certain embodiments, A is optionally substituted alkyl, B is selected from the group consisting of a direct bond, aryl, and —O-aryl.

In certain embodiments, A is C₁₋₈ alkyl optionally substituted with one or more R^{a} groups, and B is a direct bond, wherein R^{a} is —C(═O)OR^{e}, wherein R^{e} is alkyl.

In certain embodiments, A is C₁₋₈ alkyl optionally substituted with one or more R^{a} groups, and B is 5 to 12 membered aryl, 5 to 10 membered aryl, 5 to 8 membered aryl, or 5 to 6 membered aryl. In certain embodiments, A is C₁₋₈ alkyl optionally substituted with one or more R^{a} groups, and B is phenyl.

In certain embodiments, A is C₁₋₈ alkyl optionally substituted with one or more R^{a} groups, and B is —O-aryl. In certain embodiments, A is C₁₋₈ alkyl optionally substituted with one or more R^{a} groups, and B is —O-phenyl.

In some embodiments, A is —(CH₂CH₂O)ₘ—, wherein m is an integer from 0 to 4, and B is selected from the group consisting of a direct bond, alkyl, cycloalkyl, heterocyclyl, aryl, and heteroaryl.

In certain embodiments, A is —(CH₂CH₂O)ₘ—, wherein m is an integer from 0 to 4, and B is selected from the group consisting of a direct bond, alkyl, aryl and heteroaryl.

In certain embodiments, A is —(CH₂CH₂O)ₘ—, wherein m is an integer from 0 to 4, and B is a direct bond.

In certain embodiments, A is —(CH₂CH₂O)ₘ—, wherein m is an integer from 0 to 4, and B is C₁₋₆ alkyl, C₁₋₅ alkyl, C₁₋₄ alkyl, C₁₋₃ alkyl, or C₁₋₂ alkyl. In certain embodiments, A is —(CH₂CH₂O)ₘ—, wherein m is an integer from 0 to 4, and B is ethyl.

In certain embodiments, A is —(CH₂CH₂O)ₘ—, wherein m is an integer from 0 to 4, and B is 5 to 12 membered aryl, 5 to 10 membered aryl, 5 to 8 membered aryl, or 5 to 6 membered aryl. In certain embodiments, A is —(CH₂CH₂O)ₘ—, wherein m is an integer from 0 to 4, and B is phenyl.

In certain embodiments, A is —(CH₂CH₂O)ₘ—, wherein m is an integer from 0 to 4, and B is 5 to 12 membered heteroaryl, 5 to 10 membered heteroaryl, 5 to 8 membered heteroaryl, or 5 to 6 membered heteroaryl. In certain embodiments, A is —(CH₂CH₂O)ₘ—, wherein m is an integer from 0 to 4, and B is pyridinyl.

In some embodiment, C is a direct bond

In some embodiment, C is —C(═O)—.

In some embodiments, C is —C(═O)N(R²)—.

In some embodiment, C is —N(R²)C(═O)—.

In some embodiment, C is —[CH₂NHC(═O)]ₙ—.

In some embodiment, C is —[NHC(═O)CH₂]ₙ—.

In some embodiment, C is —NH(CH₂)ₚC(═O)—.

In some embodiment, A is an alkyl, B is selected from the group consisting of a direct bond, cycloalkyl, heterocyclyl, aryl, heteroaryl, —O-cycloalkyl, —O— heterocyclyl, —O-aryl, and —O-heteroaryl, and C is selected from the group consisting of a direct bond, —C(═O)—, —N(R²)C(═O)—, —[CH₂NHC(═O)]ₙ—, —[NHC(═O)CH₂]ₙ— and —NH(CH₂)ₚC(═O)—.

In some embodiments, A is an alkyl, B is selected from the group consisting of a direct bond, cycloalkyl, heterocyclyl, aryl, heteroaryl, —O-cycloalkyl, —O— heterocyclyl, —O-aryl, and —O-heteroaryl, and C is a direct bond, —N(R²)C(═O)— or —[NHC(═O)CH₂]ₙ—.

In certain embodiments, A is alkyl, B is selected from the group consisting of a direct bond, aryl, and —O-aryl, and C is a direct bond or —N(R²)C(═O)—.

In certain embodiments, A is C₁₋₈ alkyl optionally substituted with one or more R^{a} groups, B is a direct bond, and C is a direct bond or —N(R²)C(═O)—, wherein R^{a} is —C(═O)OR^{e}, wherein R^{e} is alkyl.

In certain embodiments, A is C₁₋₈ alkyl optionally substituted with one or more R^{a} groups, B is 5 to 12 membered aryl, 5 to 10 membered aryl, 5 to 8 membered aryl, or 5 to 6 membered aryl, and C is a direct bond or —N(R²)C(═O)—, wherein R^{a} is —C(═O)OR^{e}, wherein R^{e} is alkyl.

In certain embodiments, A is C₁₋₈ alkyl optionally substituted with one or more R^{a} groups, B is phenyl, and C is a direct bond or —N(R²)C(═O)—, wherein R^{a} is —C(═O)OR^{e}, wherein R^{e} is alkyl.

In certain embodiments, A is C₁₋₈ alkyl optionally substituted with one or more R^{a} groups, B is —O-aryl, and C is a direct bond, wherein R^{a} is —C(═O)OR^{e}, wherein R^{e} is an alkyl.

In certain embodiments, A is C₁₋₈ alkyl optionally substituted with one or more R^{a} groups, B is —O-phenyl, and C is a direct bond, wherein R^{a} is —C(═O)OR^{e}, wherein R^{e} is alkyl.

In some embodiments, A is —(CH₂CH₂O)ₘ—, B is selected from the group consisting of a direct bond, alkyl, cycloalkyl, heterocyclyl, aryl, and heteroaryl, and C is a direct bond or —N(R²)C(═O)—.

In some embodiments, A is —(CH₂CH₂O)ₘ—, wherein m is 1, 2, 3, or 4, B is selected from the group consisting of a direct bond, alkyl, aryl and heteroaryl, and C is a direct bond.

In some embodiments, A is —(CH₂CH₂O)ₘ—, wherein m is 1, 2, 3, or 4, B is a direct bond, and C is a direct bond.

In some embodiments, A is —(CH₂CH₂O)ₘ—, wherein m is 1, 2, 3, or 4, B is C₁₋₆ alkyl, C₁₋₅ alkyl, C₁₋₄ alkyl, C₁₋₃ alkyl, or C₁₋₂ alkyl, and C is a direct bond.

In some embodiments, A is —(CH₂CH₂O)ₘ—, wherein m is 1, 2, 3, or 4, B is aryl, and C is a direct bond. In certain embodiments, A is —(CH₂CH₂O)ₘ—, wherein m is 1, 2, 3, or 4, B is a phenyl, and C is a direct bond.

In some embodiments, A is —(CH₂CH₂O)ₘ—, wherein m is 1, 2, 3, or 4, B is heteroaryl, and C is a direct bond. In certain embodiments, A is —(CH₂CH₂O)ₘ—, wherein m is 1, 2, 3, or 4, B is pyridinyl, and C is a direct bond.

In some embodiments, D is a direct bond.

In some embodiments, D is alkyl. In certain embodiments, D is C₁₋₆ alkyl, C₁₋₅ alkyl, C₁₋₄ alkyl, C₁₋₃ alkyl, or C₁₋₂ alkyl.

In some embodiments, D is aryl. In certain embodiments, D is 5 to 12 membered aryl, 5 to 10 membered aryl, 5 to 8 membered aryl, or 5 to 6 membered aryl. In certain embodiments, D is phenyl.

In some embodiments, the linker provided herein comprises a structure of formula (Ia) to (Im):

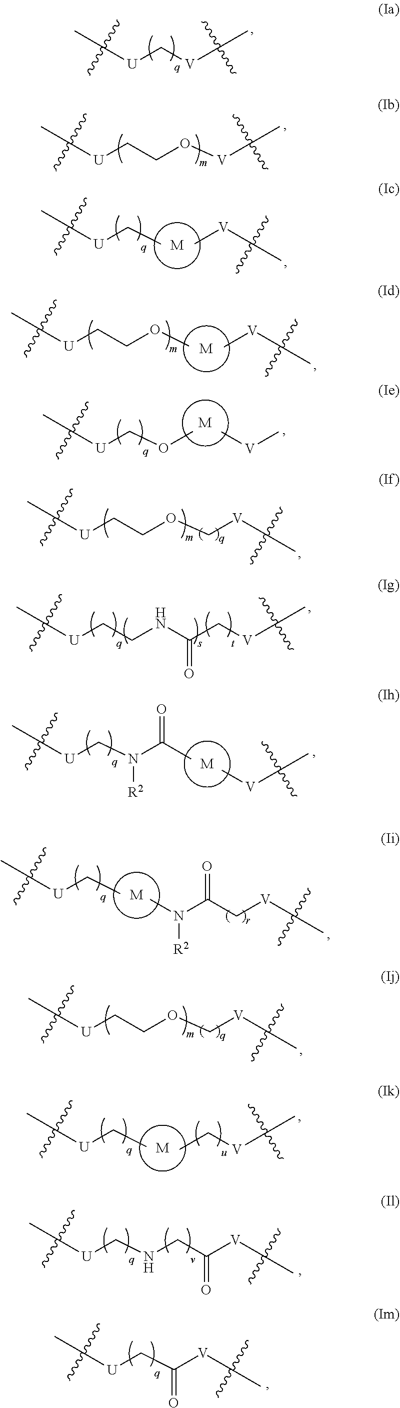

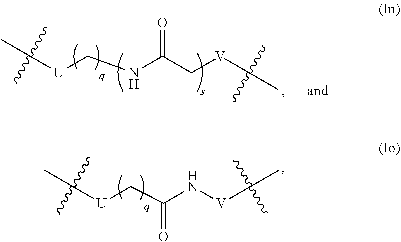

wherein,
U and V are defined as supra;
M is selected from the group consisting of cycloalkyl, heterocyclyl, aryl, and heteroaryl, each of which is optionally substituted with one or more R^{b} groups; each of

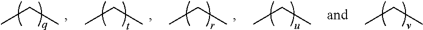

is optionally substituted with —C(═O)OCH₃; and
q, r, s, t, u and v are independently integer from 0 to 4.

In some embodiments, M is selected from the group consisting of cyclohexyl, phenyl, pyridinyl, thiazolyl, adamantyl and 2,5-diaza-bicyclo[2.2.1]heptanyl.

In some embodiments, the linker provided herein comprises a structure selected from the group consisting of:

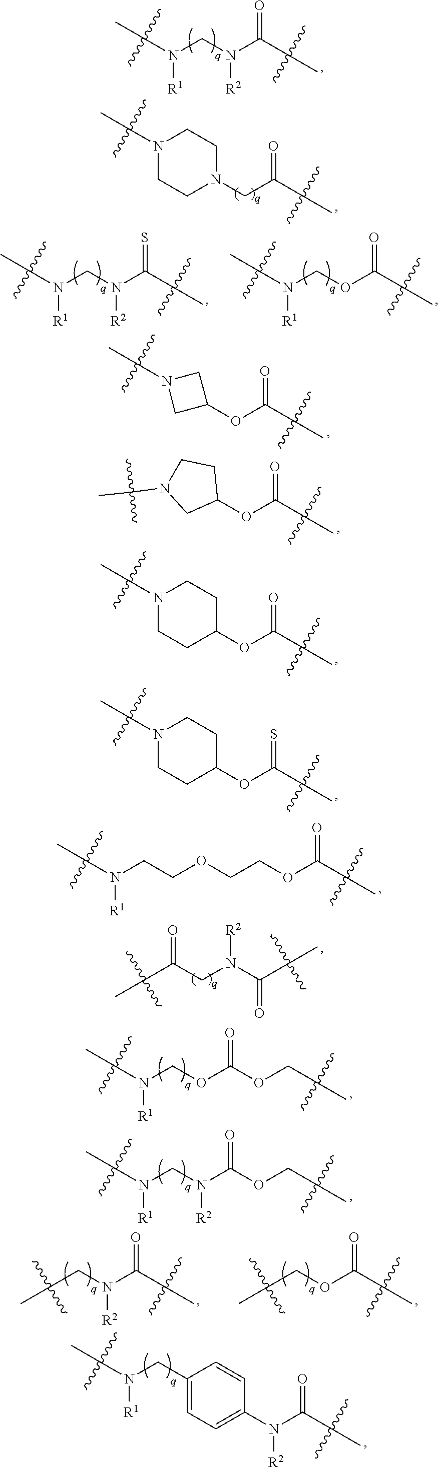

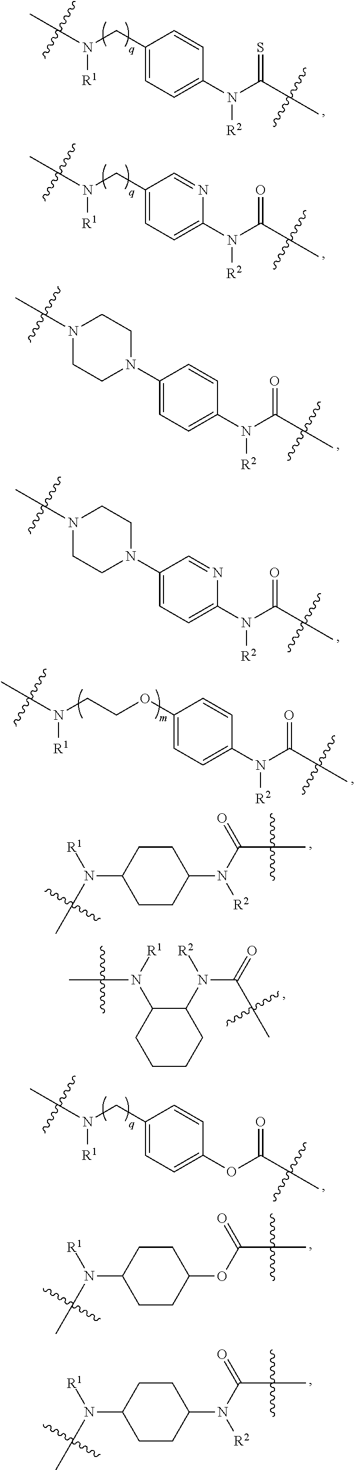

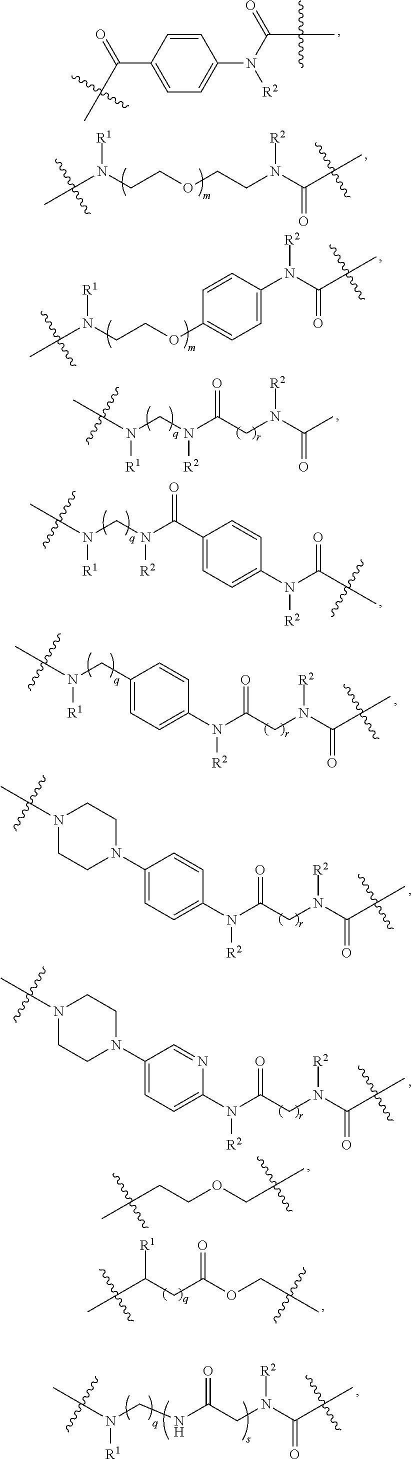

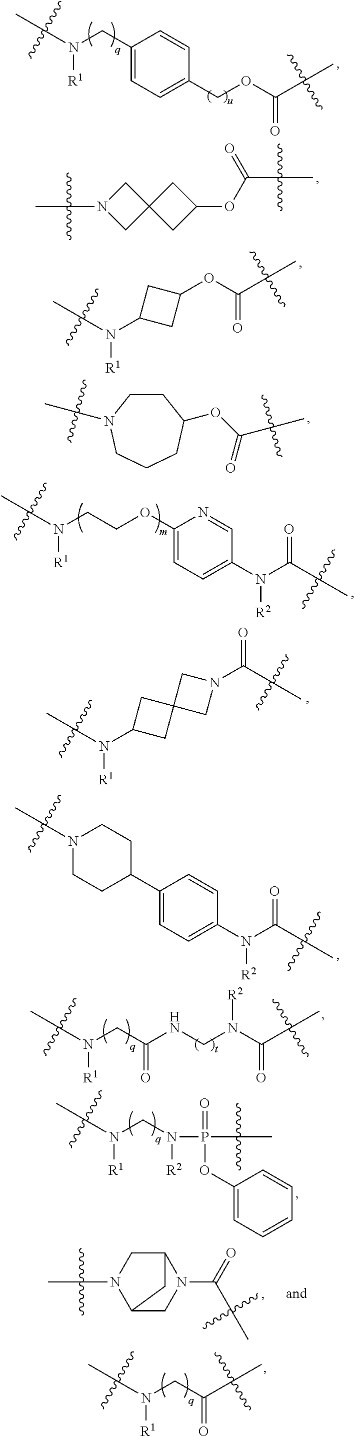

wherein each of

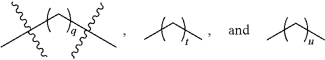

and is optionally substituted with —C(═O)OCH₃.

### Drug Delivery System

In an aspect of the present disclosure, the therapeutic agent is attached to the biopolymer via a linker, thereby providing the drug delivery system for local delivery of the therapeutic agent to target sites.

The biopolymer of the drug delivery system provided herein may have one or more therapeutic agents conjugated via the linker. The biopolymer may be conjugated to the one or more therapeutic agents via one or more linkers at hydroxyl group, carboxylic group, and/or amino group in the backbone of the biopolymer.

The drug delivery system of the present disclosure is obtained by conjugation between the biopolymer and the therapeutic agent by means of the linker through the formation of linkages between the biopolymer and the linker and linkage between the therapeutic agent and the linker.

In some embodiments, a reactive functional group of the linker, may first react with the BG2 of the therapeutic agent to form a linkage between the therapeutic agent and the linker, thereby providing a therapeutic agent-linker conjugate. The therapeutic agent-linker conjugate, which contains another reactive functional group at the terminal of the linker, may subsequently react with the BG1 of the biopolymer to form a linkage between the biopolymer and the linker, thereby providing the drug delivery system of the present disclosure.

In certain embodiments, it is possible to first react the BG1 of the biopolymer with a reactive functional group of the linker to form a biopolymer-linker conjugate, and subsequently react the BG2 of the therapeutic agent with another functional group of the linker in the biopolymer-linker conjugate, thereby providing the drug delivery system of the present disclosure.

In some embodiments, the biopolymer selected for the drug delivery system provided herein is HA, and the therapeutic agent selected for the drug delivery system provided herein is Tofacitinib.

In certain embodiments, the drug delivery system provided herein is selected from the group consisting of:

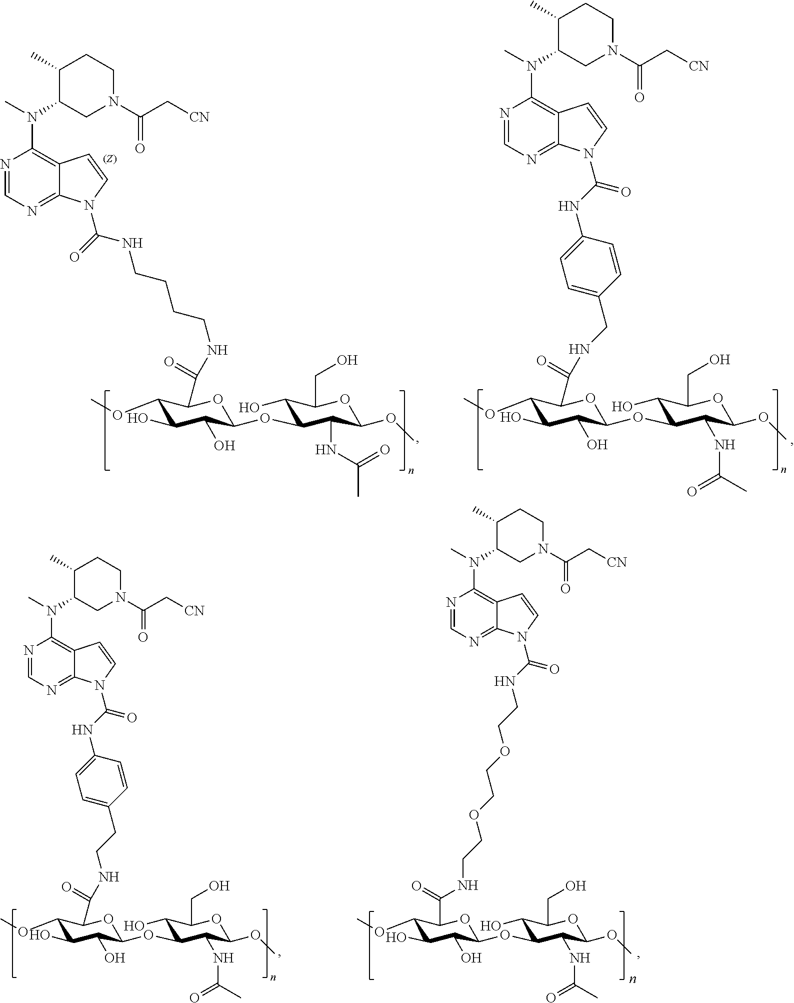

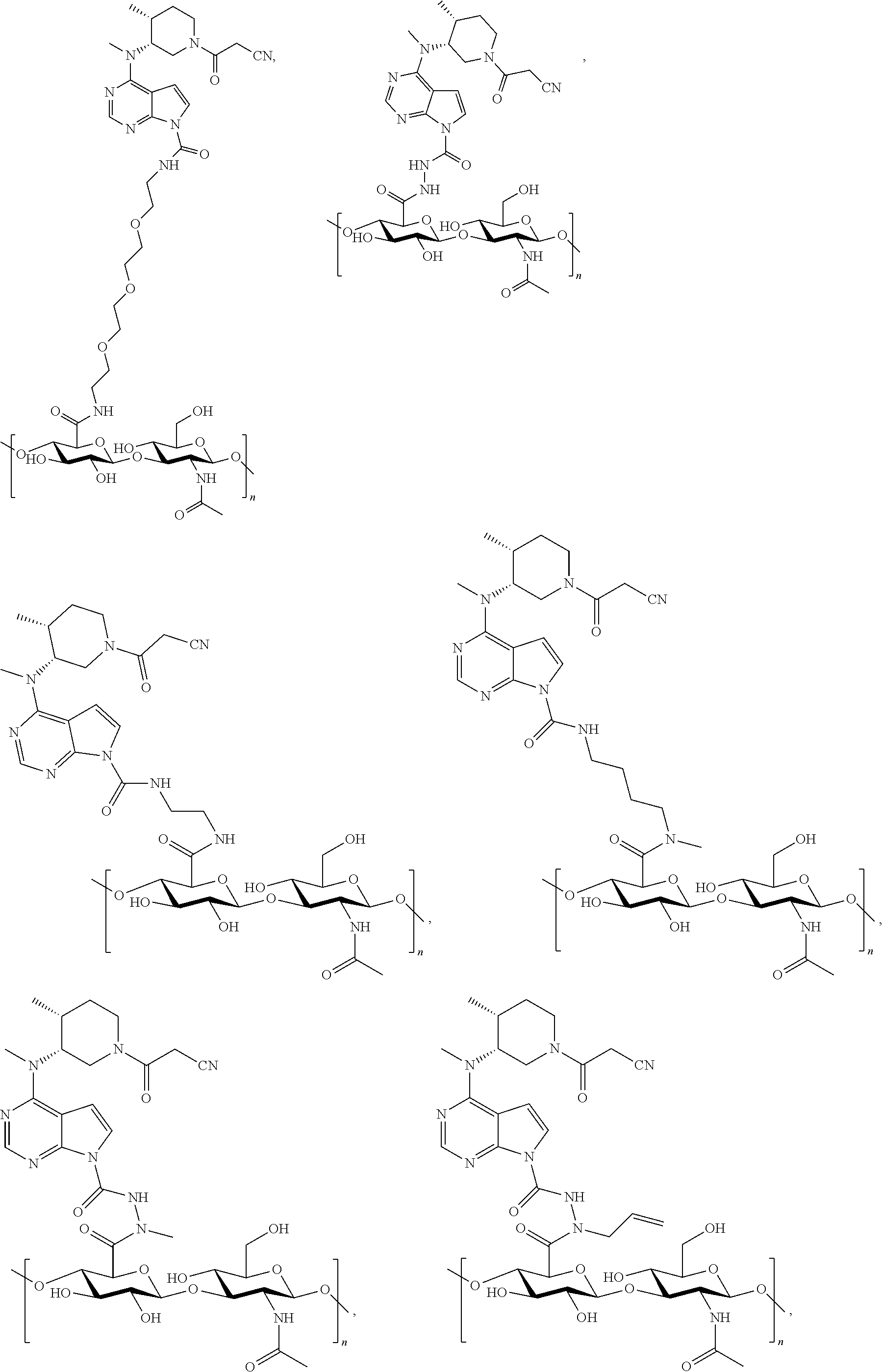

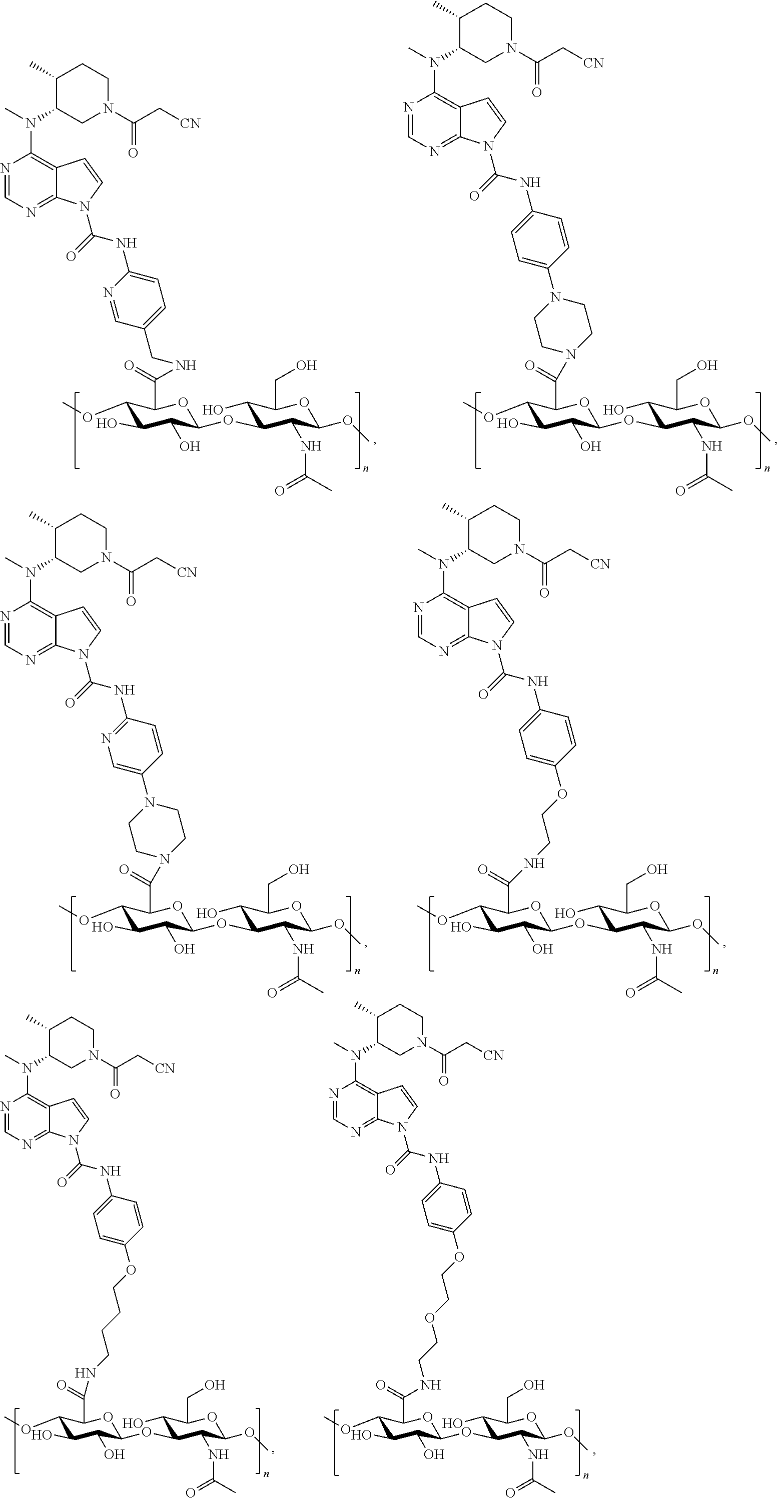

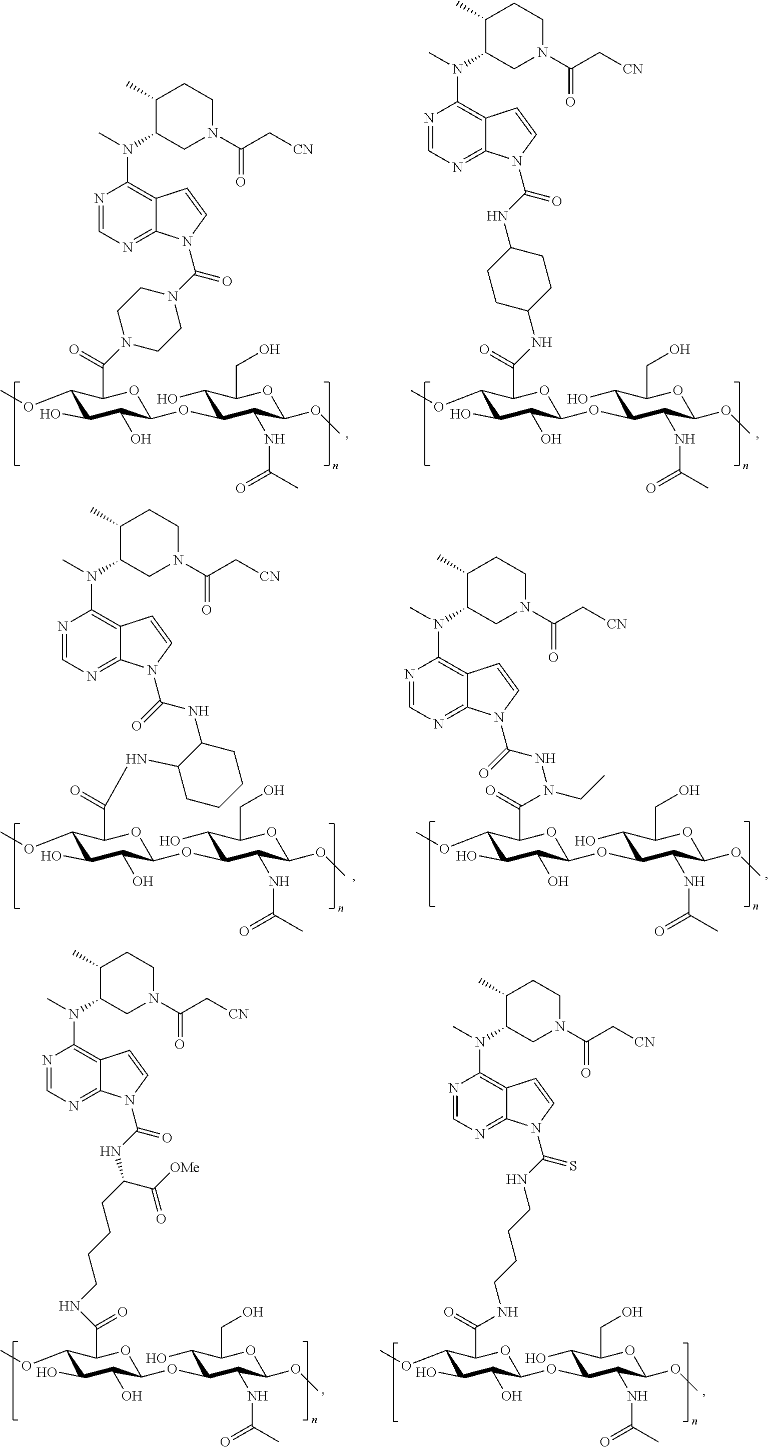

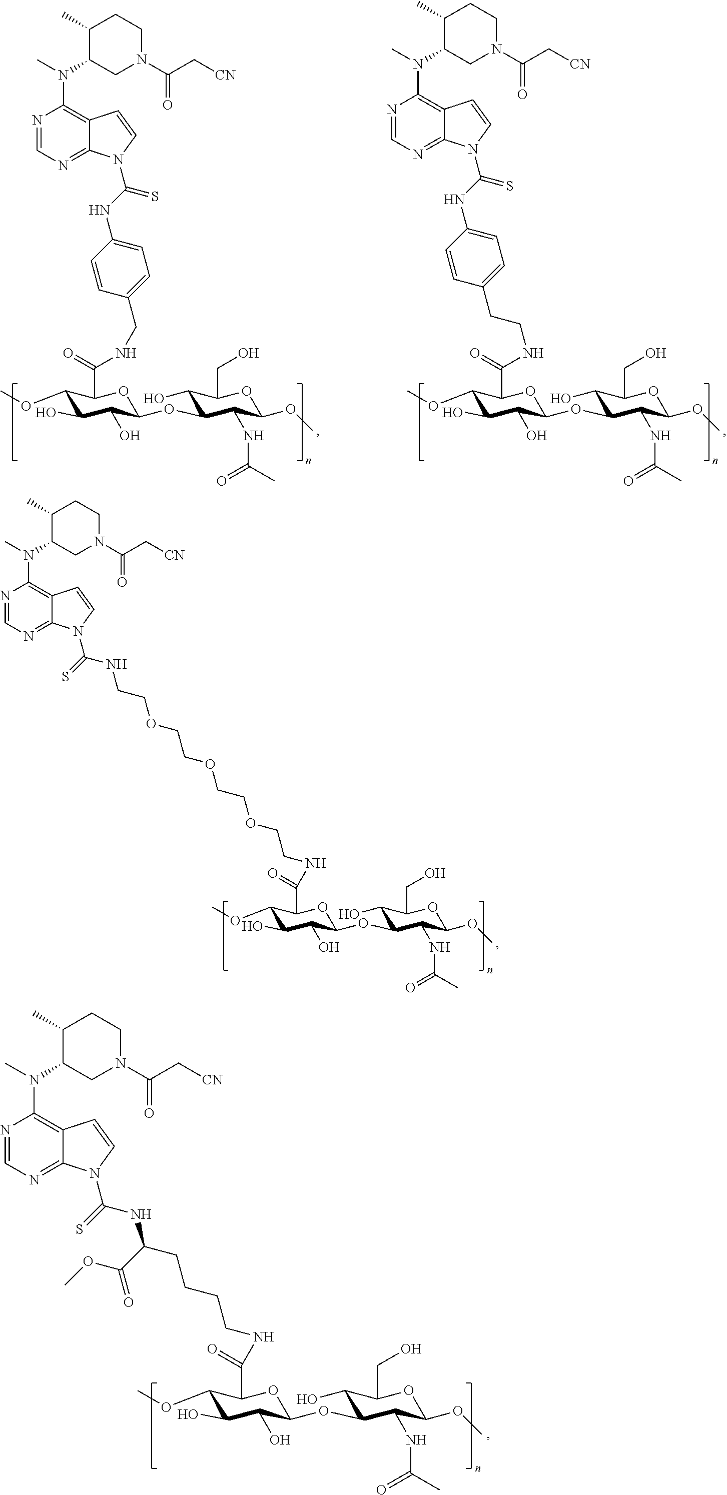

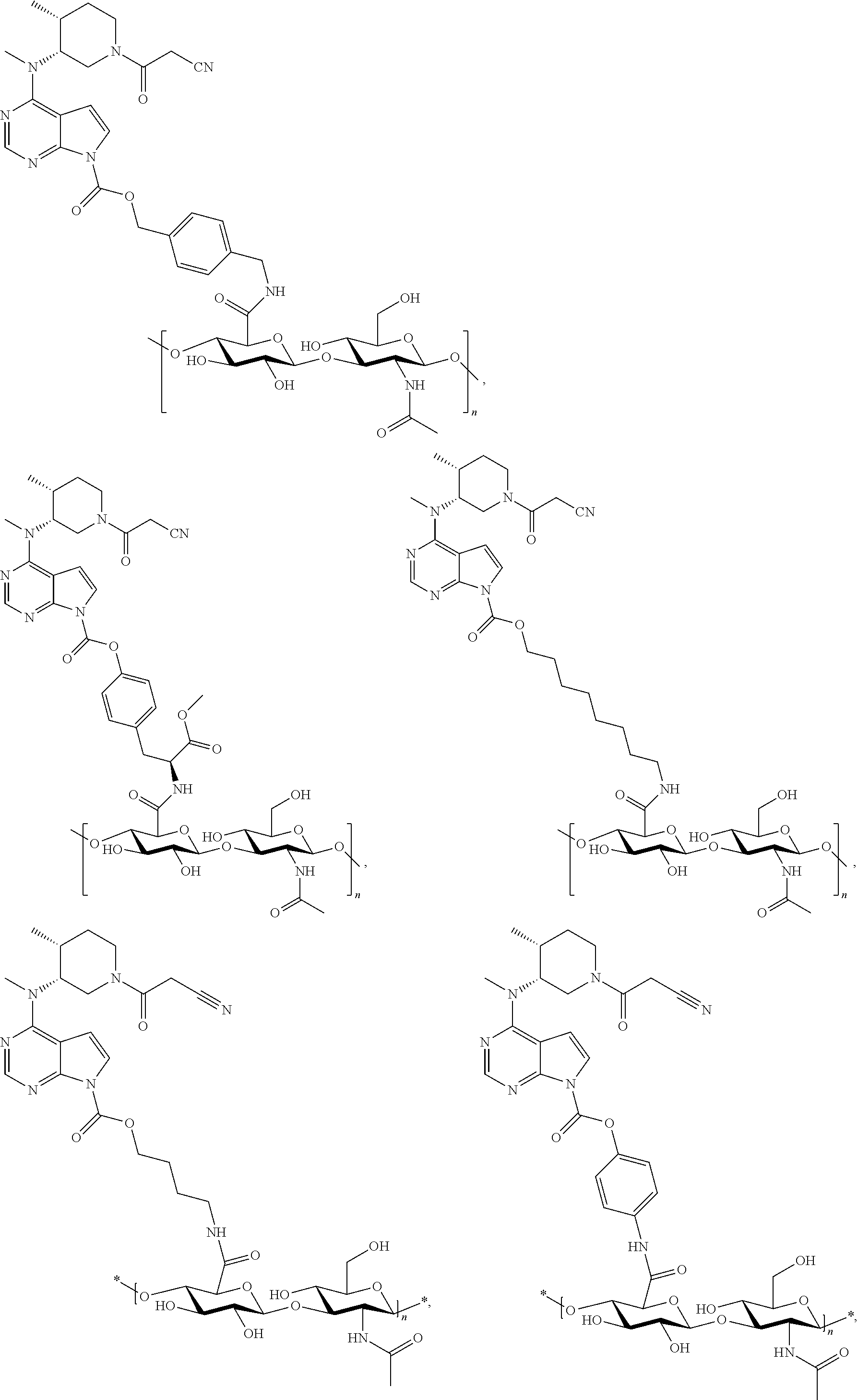

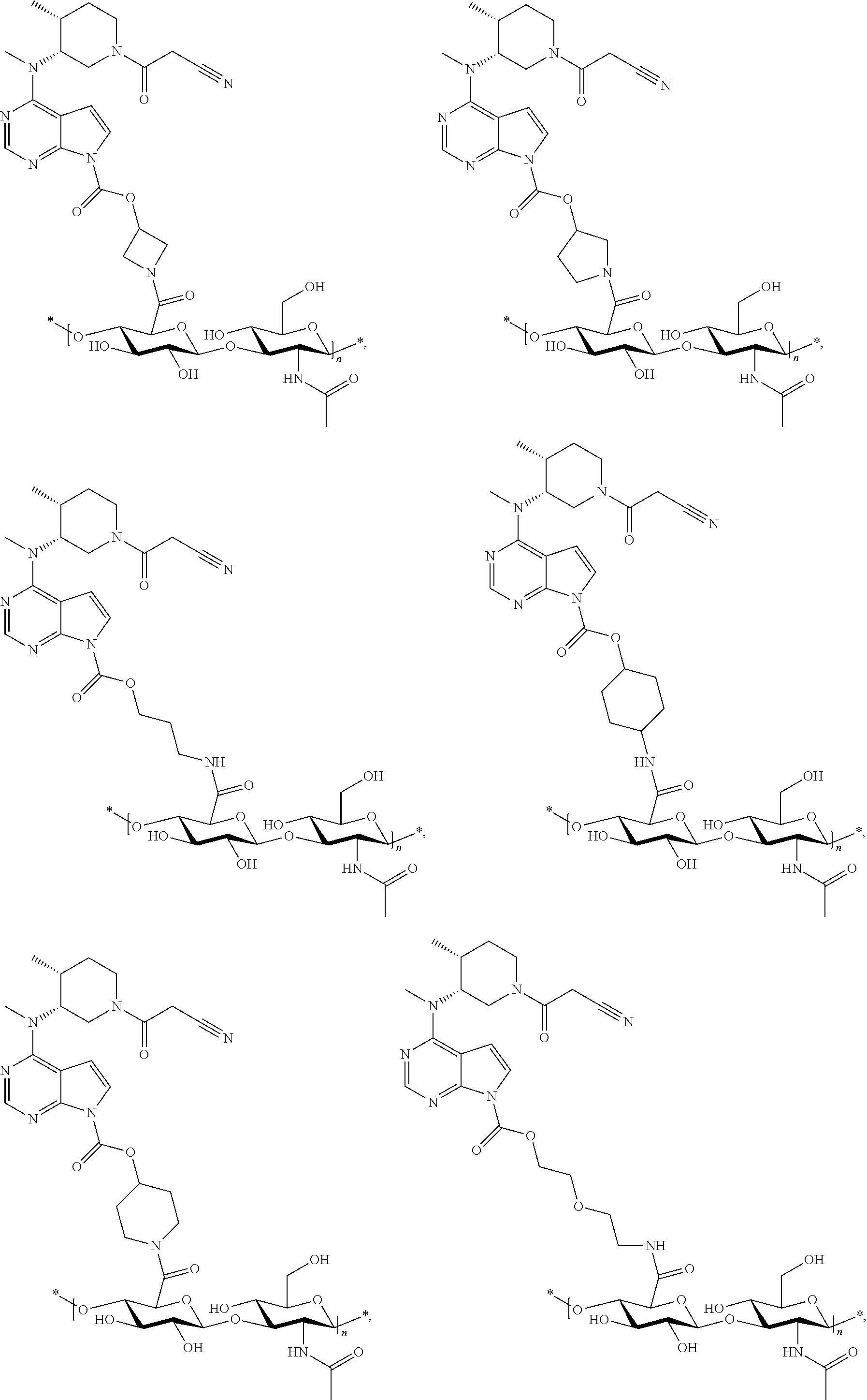

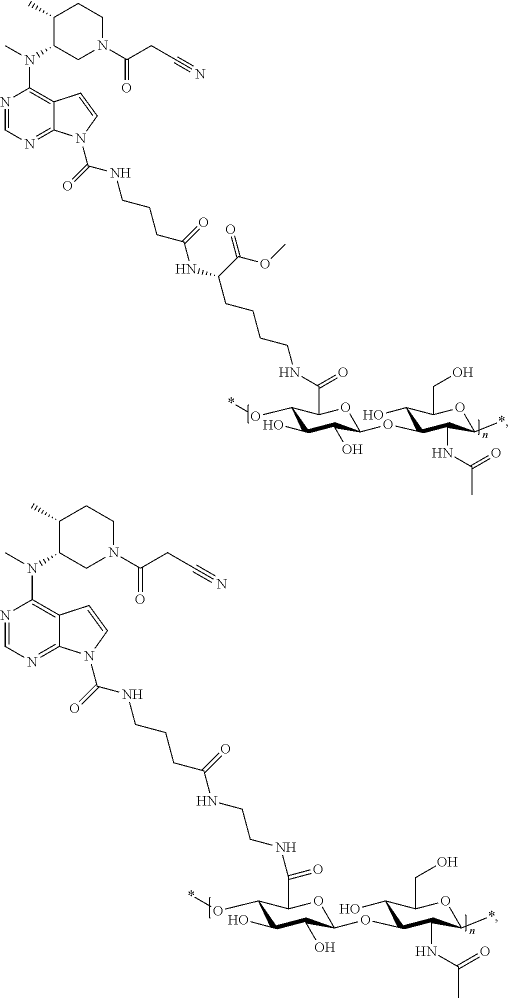

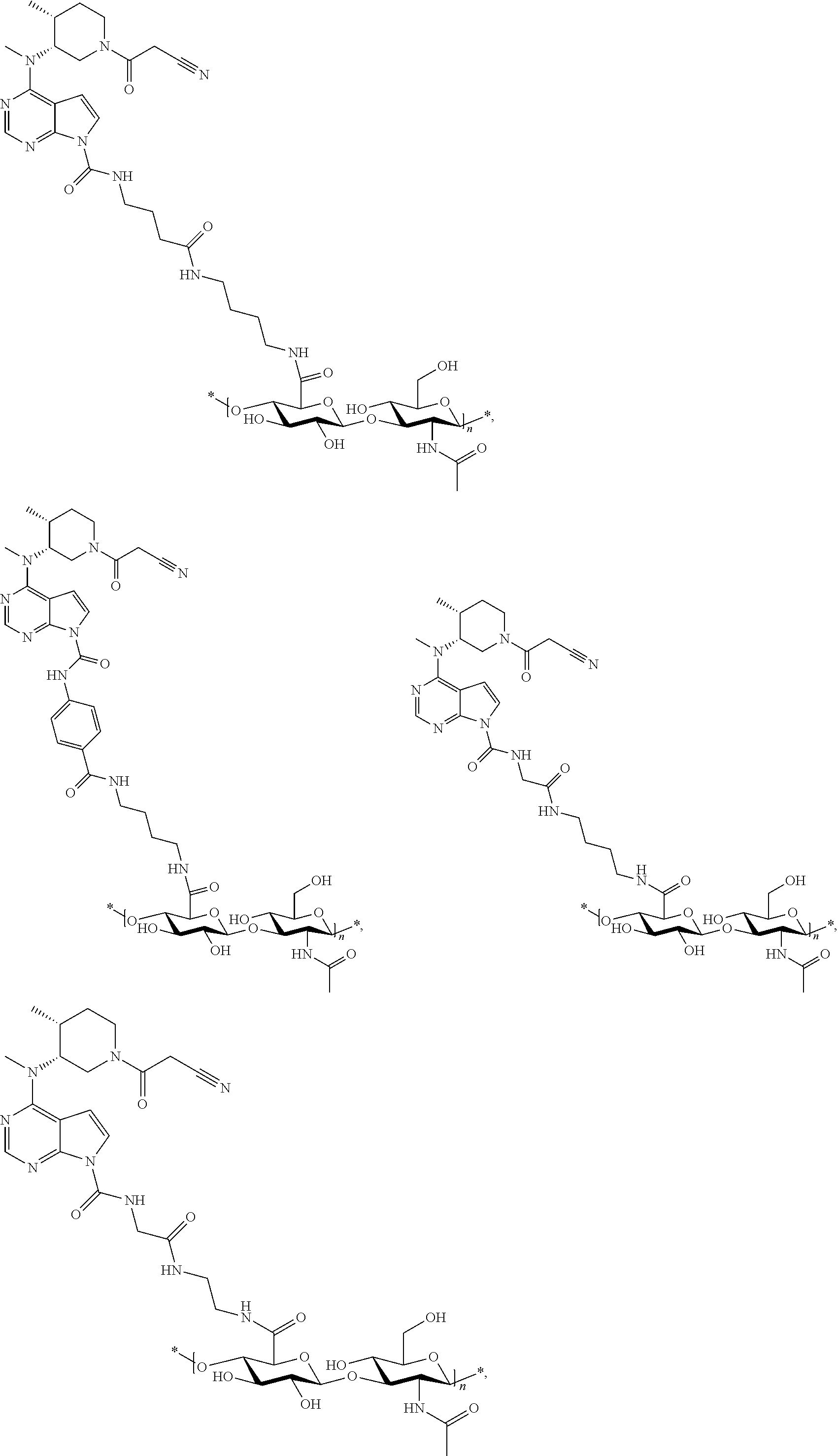

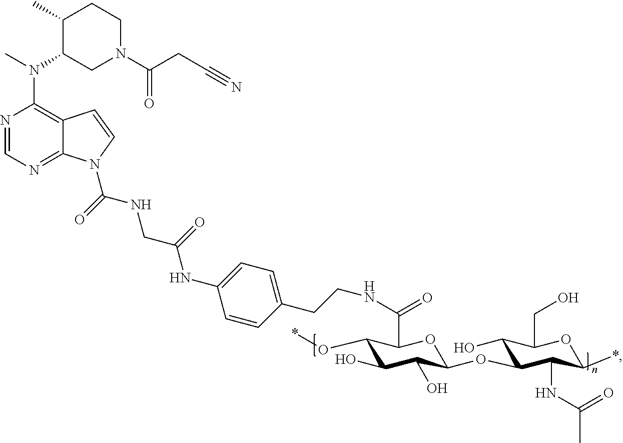

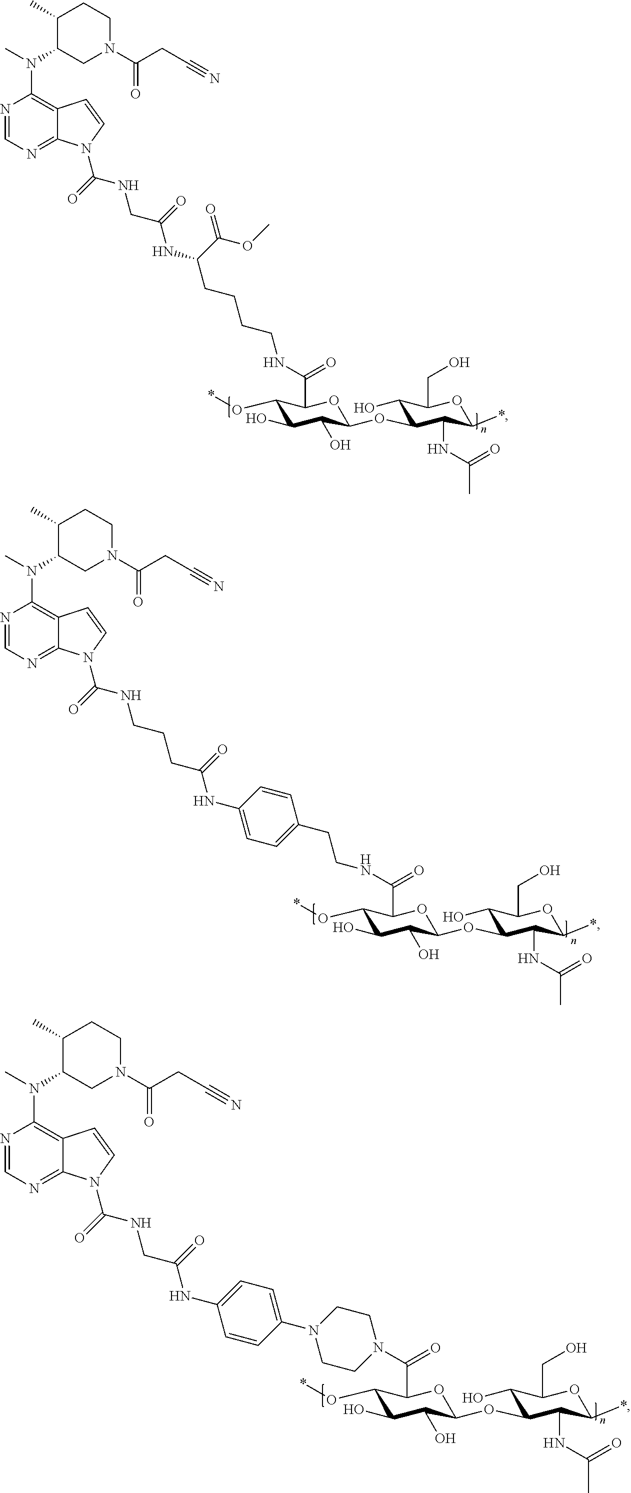

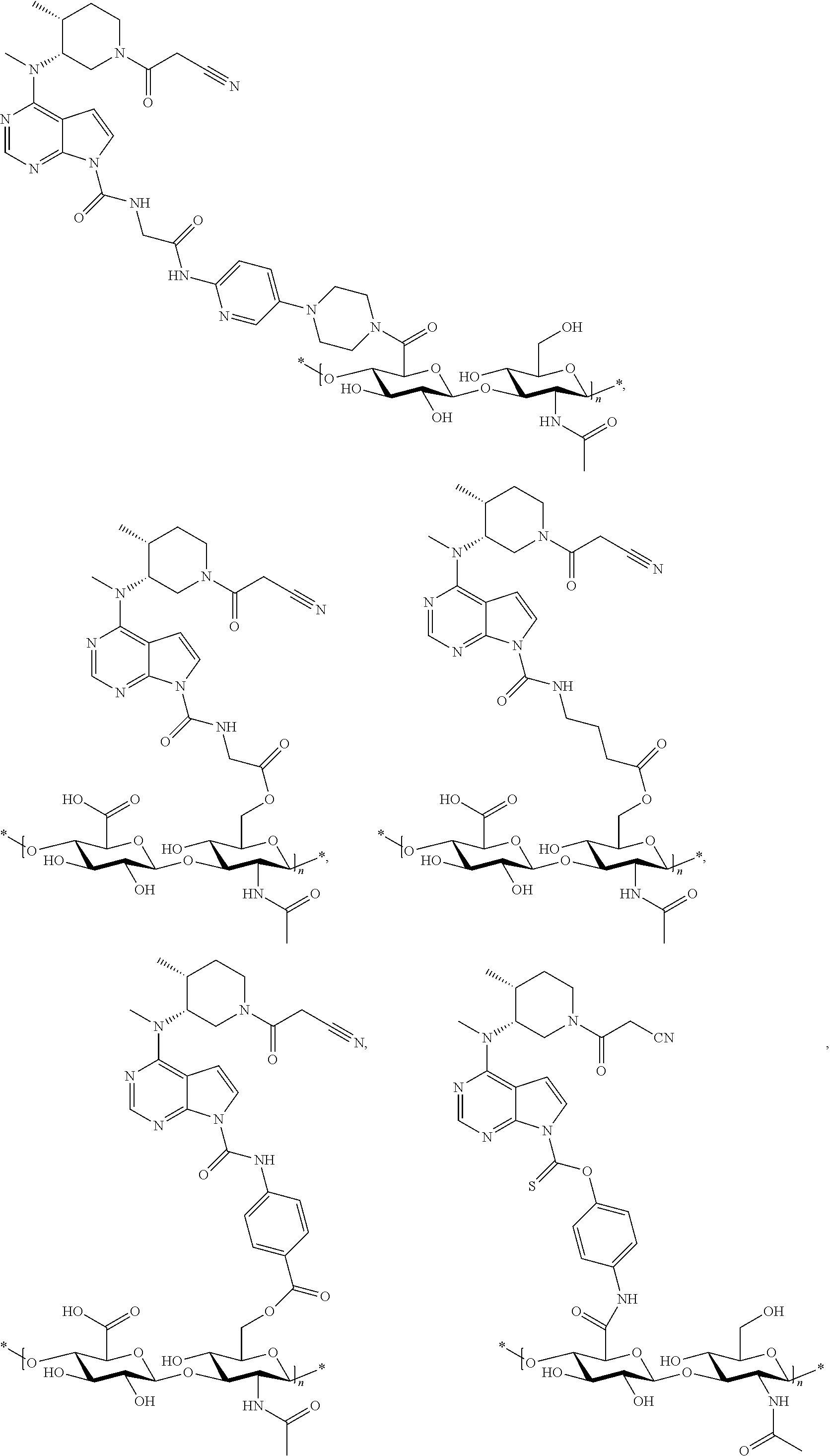

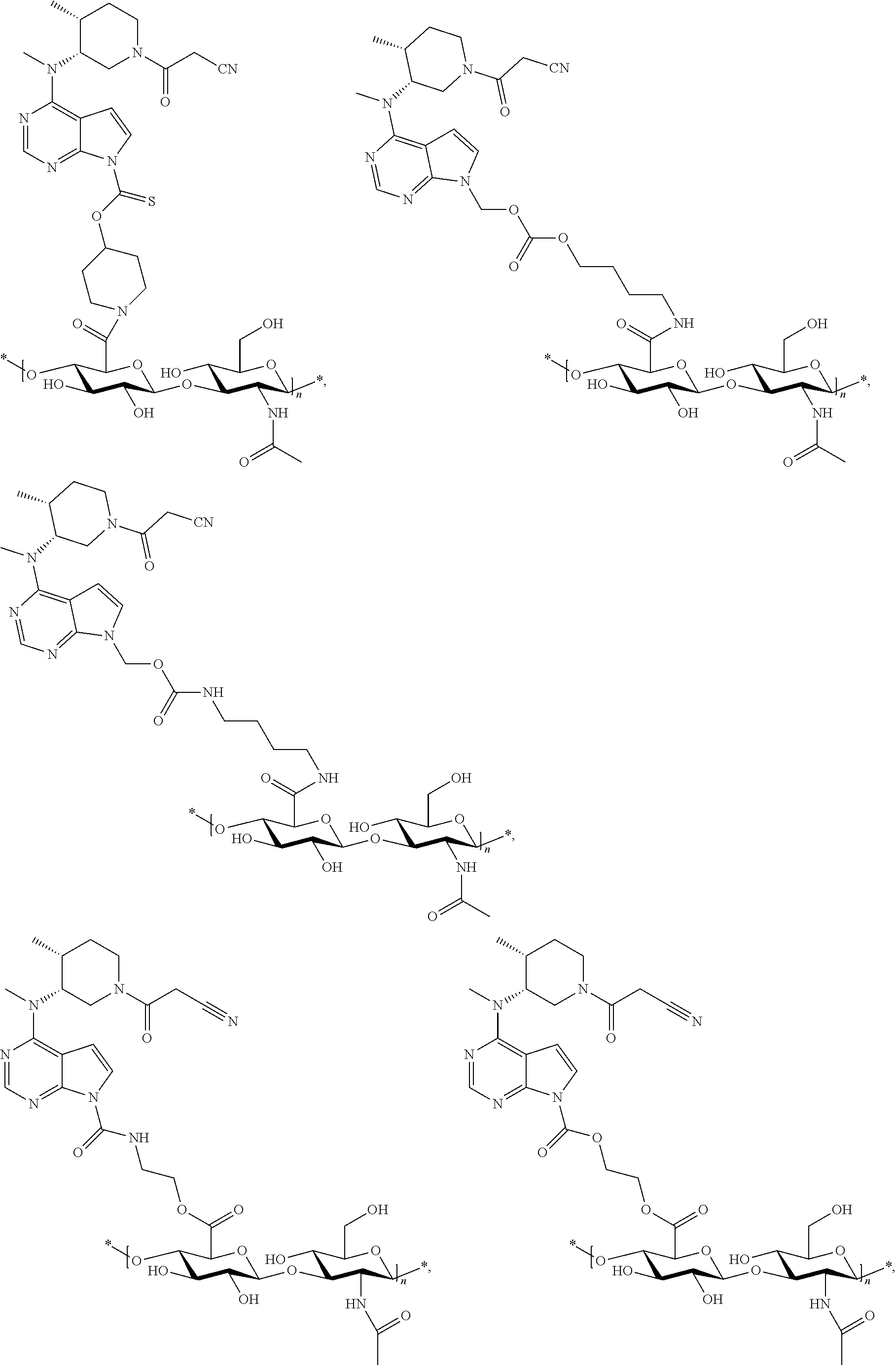

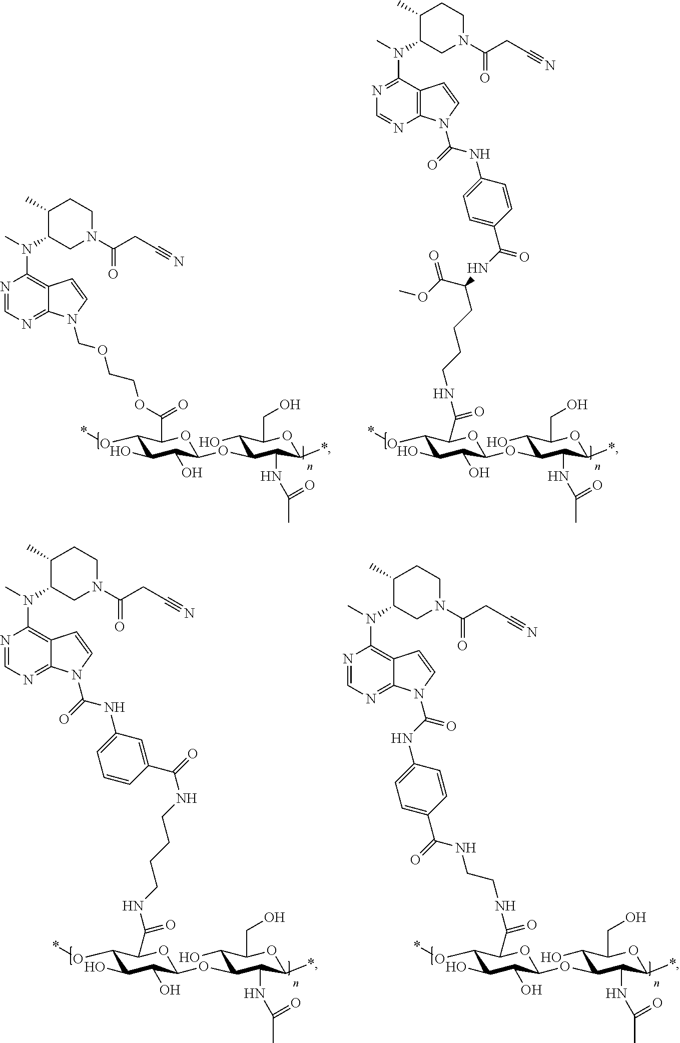

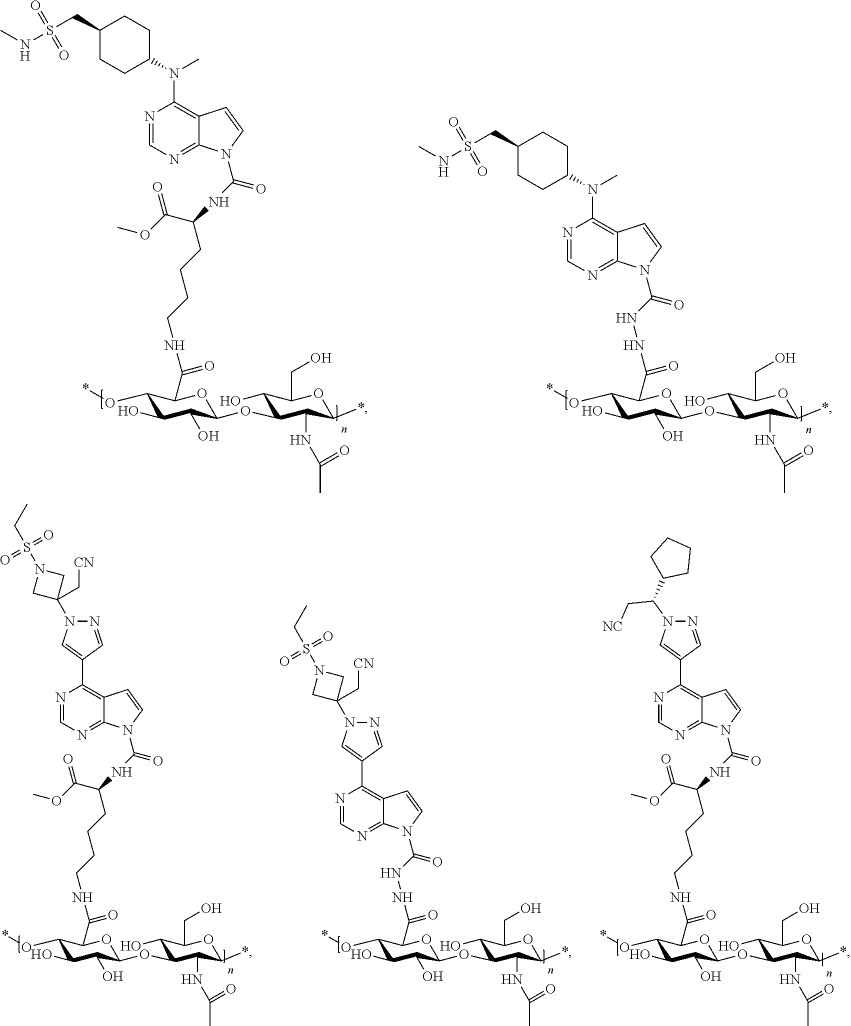

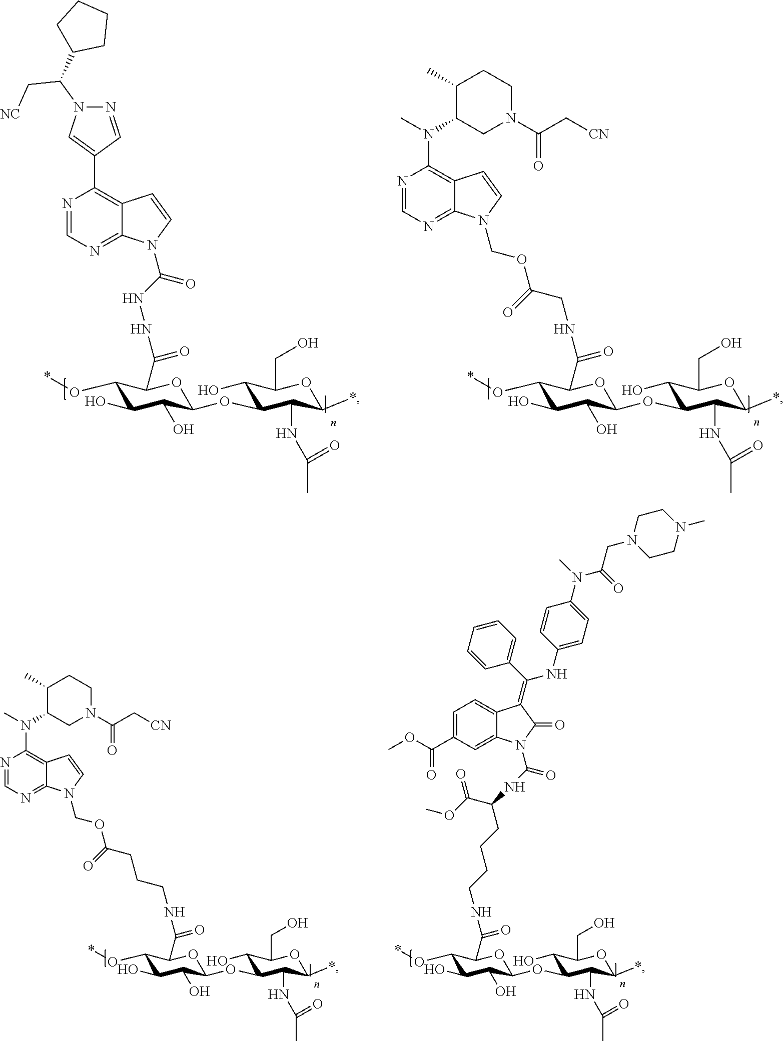

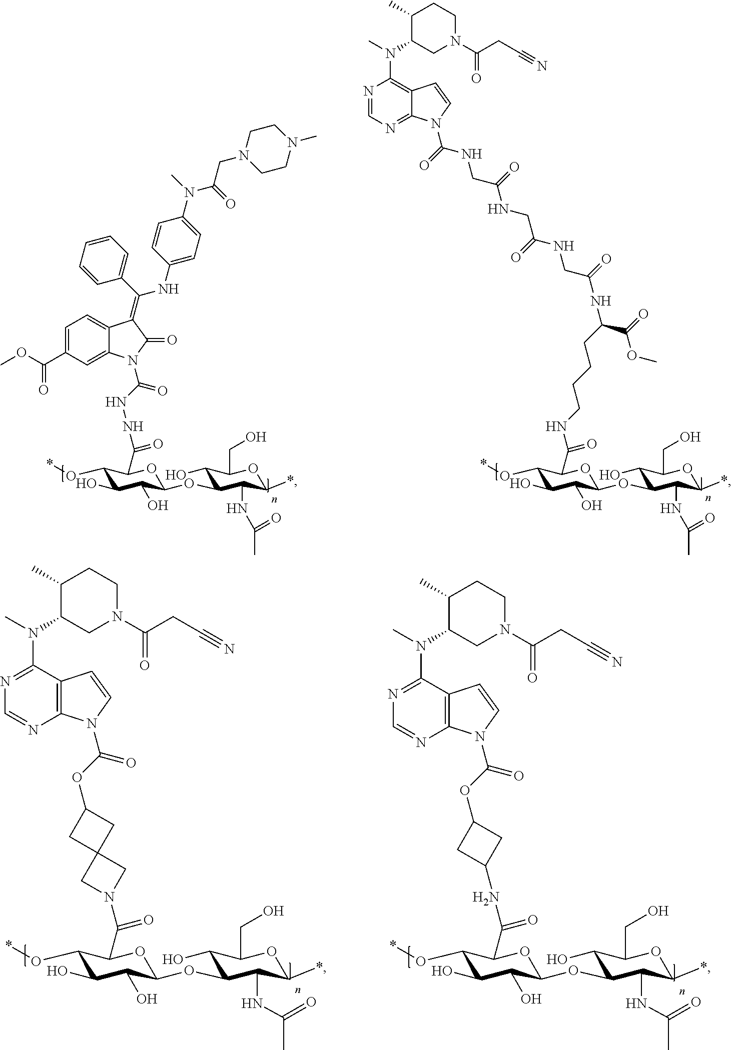

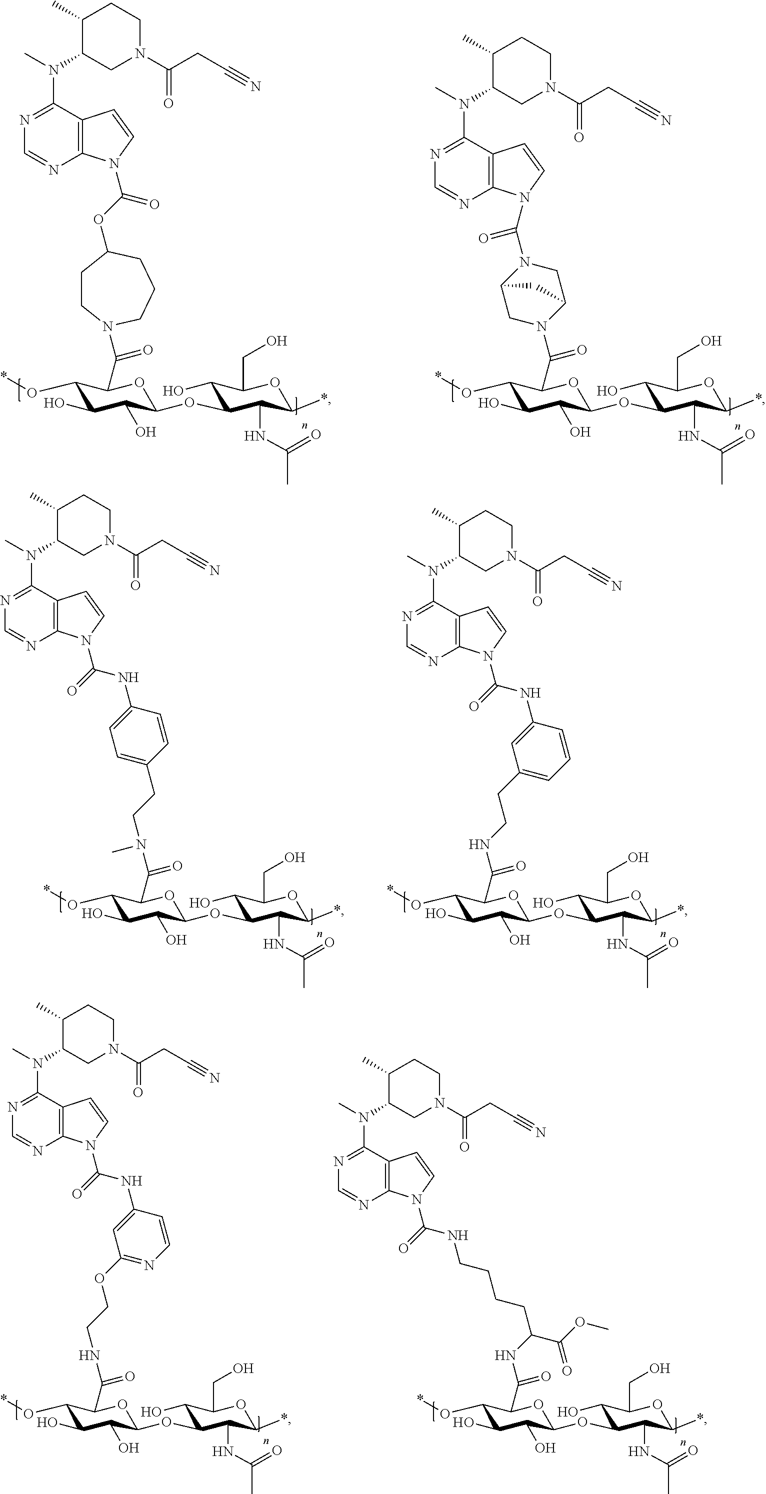

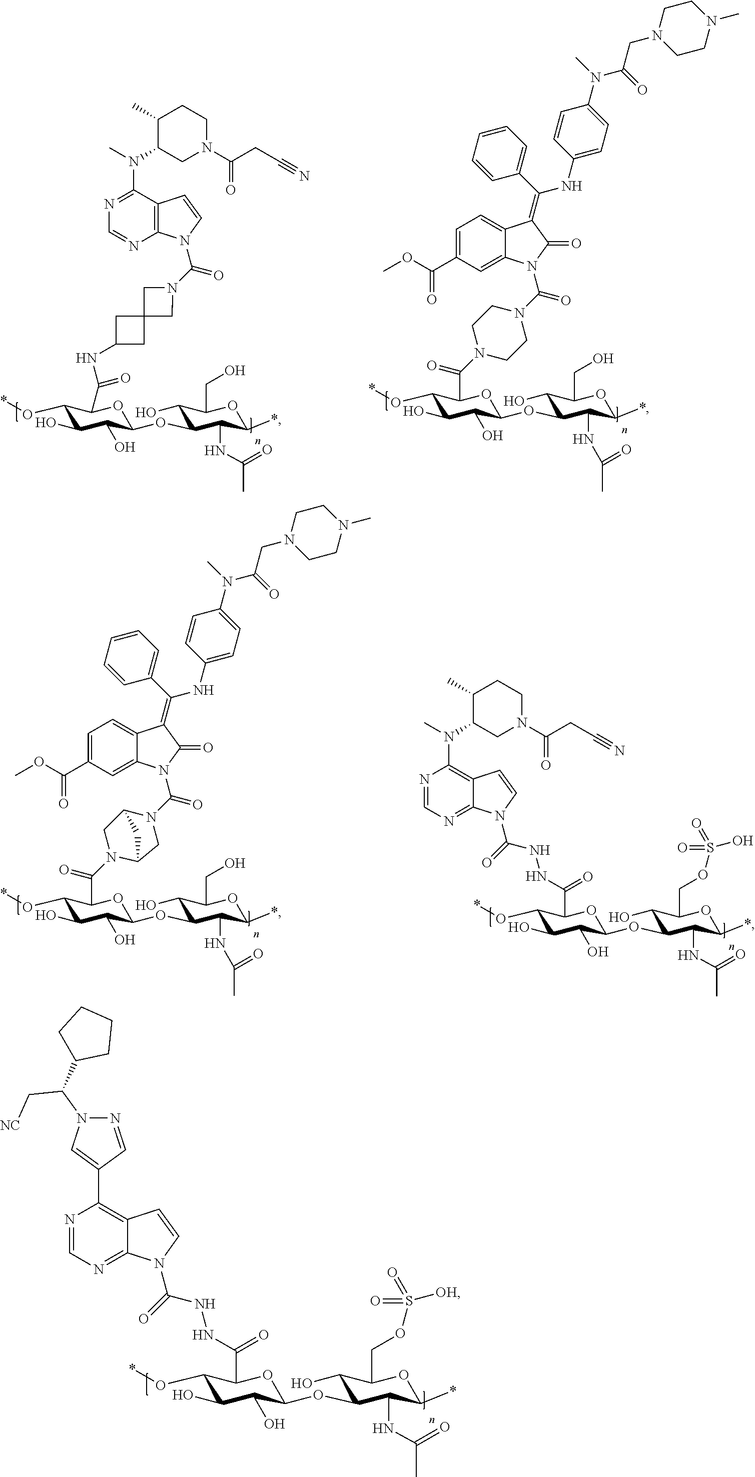

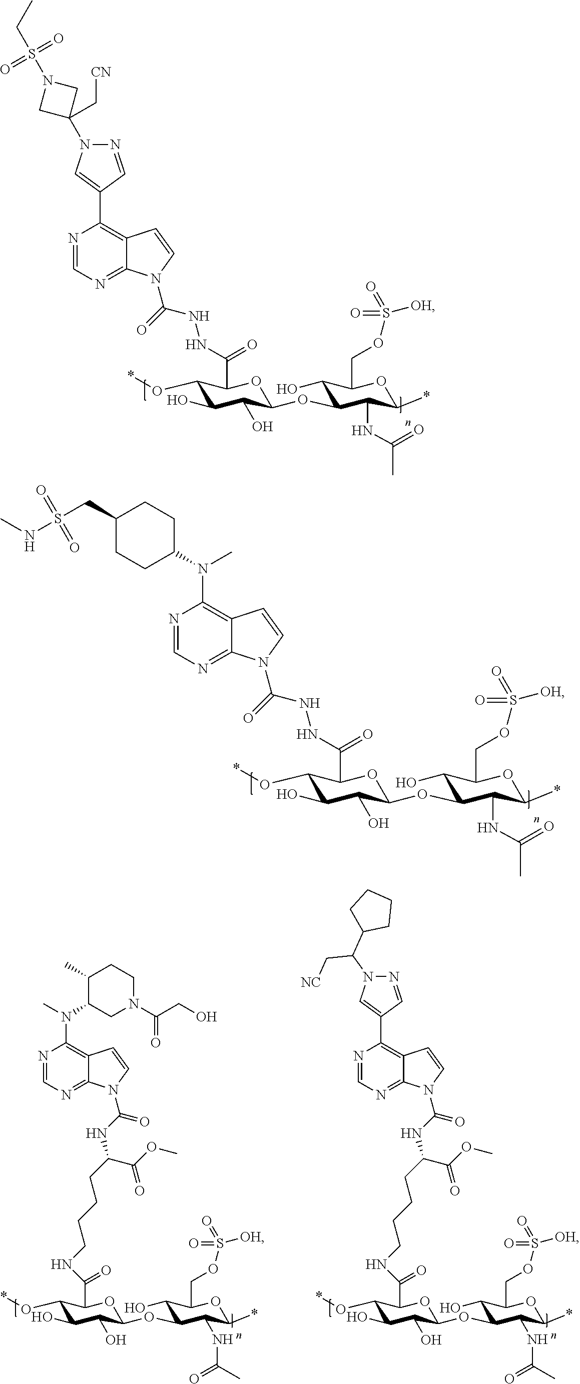

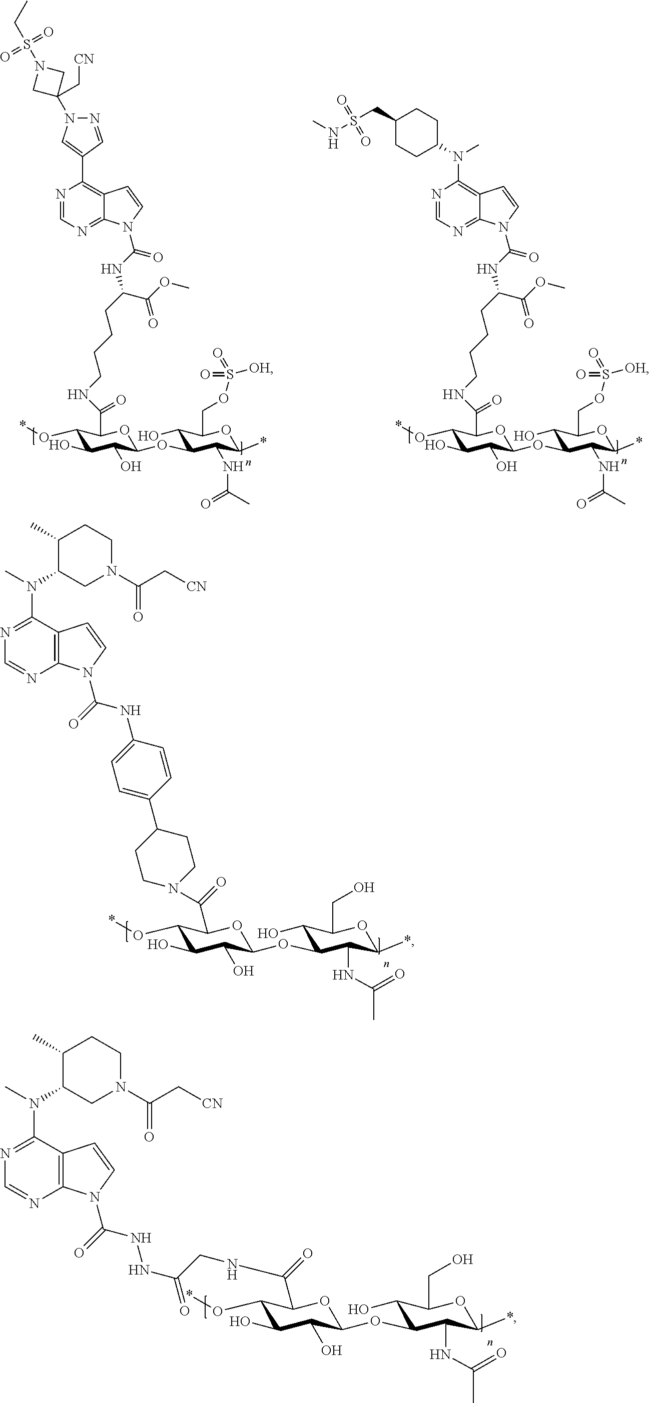

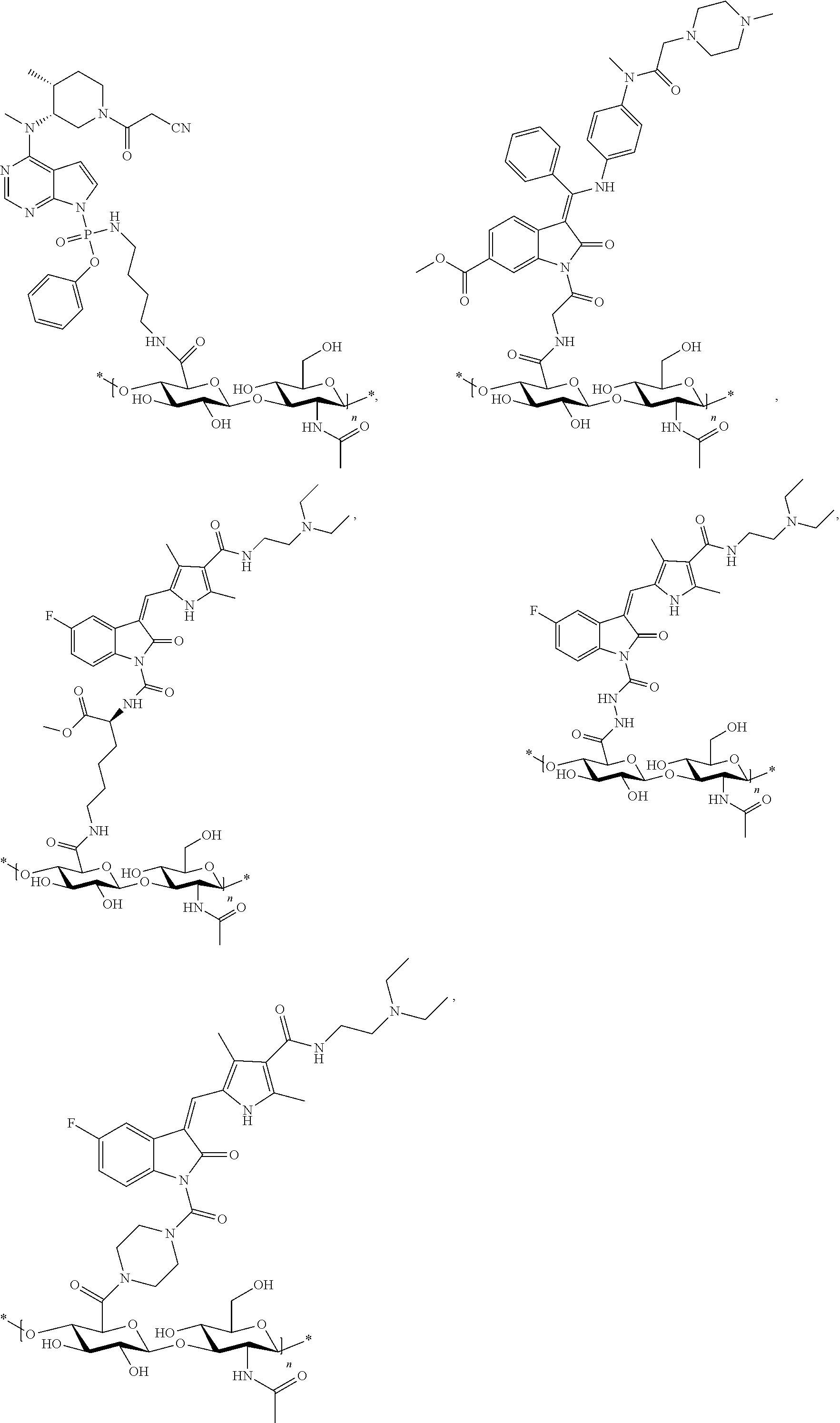

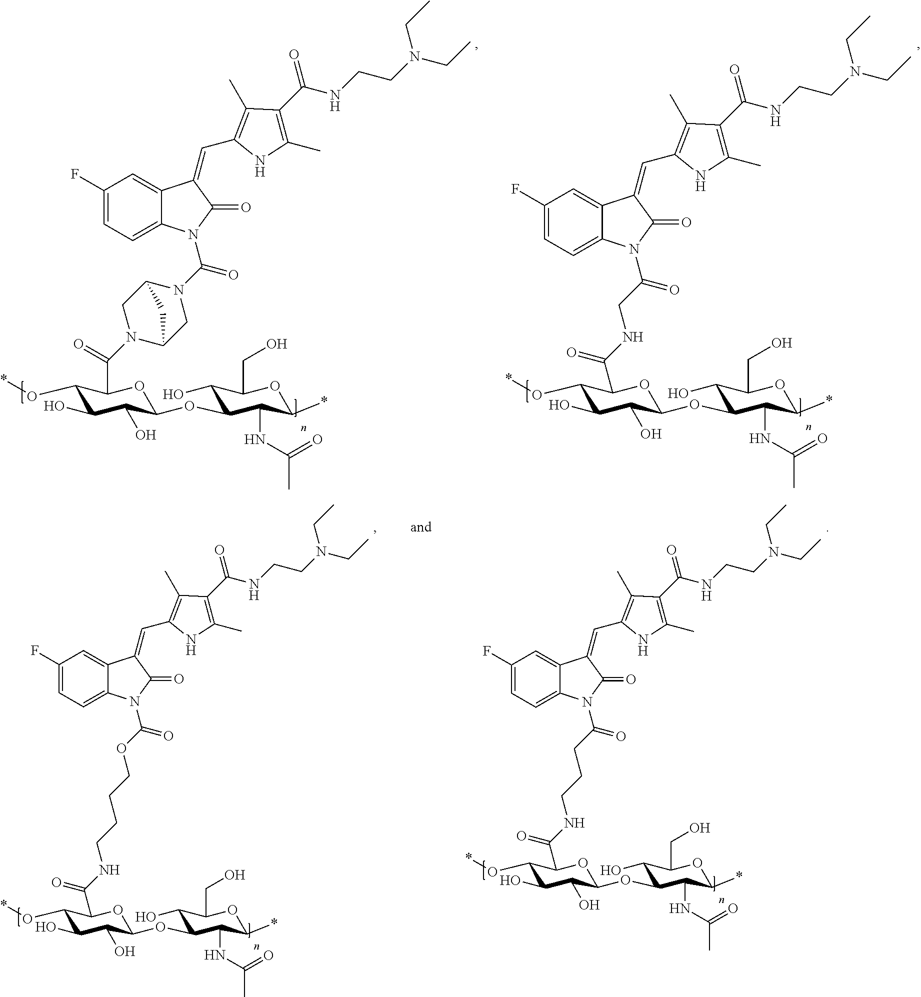

In some embodiments, the therapeutic agent can be conjugated to the biopolymer via the linker with a drug substitution rate to the biopolymer (DSR) as measured by NMR of at least 1%, at least 2%, at least 3%, at least 5%, at least 8%, at least 10%, at least 15%, at least 20%, at least 25%, at least 30%, at least 35%, at least 40%, at least 45%, at least 50%, wherein the drug substitution rate to the biopolymer (DSR) refers to the ratio of the molar amount of groups on the biopolymer which are substituted with drugs to the total molar amount of groups on the biopolymer which are capable of being substituted with drugs.

The therapeutic agent may be released from the drug delivery system provided herein through the cleavage of the linkage between the linker and the biopolymer or the therapeutic agent. In some embodiments, the release of the therapeutic agent occurs where the linkage between the biopolymer and the linker is cleaved to release a therapeutic agent-linker conjugate, which may be considered as a prodrug. Subsequent release of the therapeutic agent from the linker may involve enzymatic or non-enzymatic cleavage of the linkage between the therapeutic agent and the linker. In some embodiments, the release of the therapeutic agent occurs where the linkage between the therapeutic agent and the linker is cleaved without or prior to the cleavage of the linkage between the biopolymer and the linker. The release of the therapeutic agent may also involve enzymatic or non-enzymatic processes.

The release of therapeutic agents may be affected by a variety of factors, for example, the selection of specific therapeutic agent, linker and the biopolymer, the administration of the drug delivery system. The present disclosure contemplates biopolymers with varying molecular weight, binding group BG1, linkage with the linker; linkers with varying reactive functional groups and subunits; and therapeutic agents with varying binding group BG2, linkage with the linker.

The present disclosure also contemplates varying local administration of the drug delivery system provided herein. In some embodiments, the drug delivery system provided herein is locally administered to a subject in need thereof. In certain embodiments, the drug delivery system provided herein is locally administered to a subject in need thereof via injection. In certain embodiments, the drug delivery system provided herein is locally administered to a subject in need thereof via oral dosage form. In certain embodiments, the drug delivery system provided herein is locally administered to a subject in need thereof via inhalation. In certain embodiments, the drug delivery system provided herein is locally administered to a subject in need thereof via implant. In certain embodiments, the drug delivery system provided herein is locally administered to a subject in need thereof via topical application. Depending on the specific therapeutic agent, linker and the biopolymer combination, release of therapeutic agents may occur in a variety of locations upon administration to a subject. For example, release of therapeutic agents may occur at a site of administration.

In some embodiments, the administration of the drug delivery system provided herein to a subject may provide release of the therapeutic agent over a period of at least a few days to at least a few months.

The release of the therapeutic agent from the drug delivery system provided herein may be characterized by the percent of the therapeutic agent released per day from the drug delivery system. In some embodiments, the release rate of the therapeutic agent may vary in a range of about 0.01% to about 20% per day, about 0.01% to about 15% per day, about 0.01% to about 10% per day, about 0.01% to about 9% per day, about 0.01% to about 8% per day, about 0.01% to about 7% per day, about 0.01% to about 6% per day, about 0.01% to about 5% per day, about 0.01% to about 4% per day, about 0.01% to about 3% per day, about 0.01% to about 2% per day, about 0.01% to about 1% per day, about 0.01% to about 0.5% per day, about 0.01% to about 0.4% per day, about 0.01% to about 0.3% per day, about 0.01% to about 0.2% per day, about 0.01% to about 0.1% per day, about 0.01% to about 0.05% per day, about 0.01% to about 0.04% per day, about 0.01% to about 0.03% per day, or about 0.01% to about 0.02% per day.

### Pharmaceutical Compositions

In a further aspect, there is provided pharmaceutical compositions comprising the drug delivery system of the present disclosure.

In another aspect, there is provided pharmaceutical compositions comprising the drug delivery system of the present disclosure, and at least one pharmaceutical acceptable excipient.

As used herein, the term “pharmaceutical composition” refers to a formulation containing the drug delivery system of the present disclosure in a form suitable for administration to a subject.

As used herein, the term “pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use.

A “pharmaceutically acceptable excipient” as used herein includes both one and more than one such excipient. The term “pharmaceutically acceptable excipient” also encompasses “pharmaceutically acceptable carrier” and “pharmaceutically acceptable diluent”.

The pharmaceutical compositions provided herein can be in any form that allows for the composition to be administered to a subject, including, but not limited to a human, and formulated to be compatible with an intended route of administration.

A variety of routes are contemplated for the pharmaceutical compositions provided herein, and accordingly the pharmaceutical composition provided herein may be supplied in bulk or in unit dosage form depending on the intended administration route. For example, for oral, buccal, and sublingual administration, powders, granules, tablets, pills, capsules, gelcaps, and caplets may be acceptable as solid dosage forms, and emulsions, syrups, elixirs, suspensions, and solutions may be acceptable as liquid dosage forms. For injection administration, gel, solutions, emulsions and suspensions may be acceptable as liquid dosage forms, and a powder suitable for reconstitution with an appropriate solution as solid dosage forms. For inhalation administration, solutions, sprays, dry powders, and aerosols may be acceptable dosage form. For topical (including buccal and sublingual) or transdermal administration, powders, sprays, ointments, pastes, creams, lotions, gels, solutions, and patches may be acceptable dosage form. For vaginal administration, pessaries, tampons, creams, gels, pastes, foams and spray may be acceptable dosage form. For implant administration, solid, semi-solid, gel may be acceptable dosage form.

In some embodiments, the pharmaceutical compositions of the present disclosure may be in a form of formulation for oral administration.

In some embodiments, the pharmaceutical compositions of the present disclosure may be in a form of formulation for injection administration.

In some embodiments, the pharmaceutical compositions of the present disclosure may be in a form of formulation for inhalation administration.

In some embodiments, the pharmaceutical compositions of the present disclosure may be in a form of formulation for topical administration.

In certain embodiments, the pharmaceutical compositions provided herein may be formulated in the form of skin patches that are well known to those of ordinary skill in the art.

Besides those representative dosage forms described above, pharmaceutically acceptable excipients and carriers are generally known to those skilled in the art and are thus included in the present disclosure. Such excipients and carriers are described, for example, in “Remingtons Pharmaceutical Sciences” Mack Pub. Co., New Jersey (1991), in “Remington: The Science and Practice of Pharmacy”, Ed. University of the Sciences in Philadelphia, 21^{st} Edition, LWW (2005), which are incorporated herein by reference.

In some embodiments, the pharmaceutical compositions of the present disclosure can be formulated as a single dose. The amount of the compounds provided herein in the single dose will vary depending on the subject treated and particular mode of administration.

In some embodiments, the pharmaceutical compositions of the present disclosure can be formulated to be administered to a subject at a time interval of a few days, a few weeks, a few months or even longer.

In a further aspect, there is also provided pharmaceutical compositions comprise the drug delivery system of the present disclosure, as two or more combination therapy.

### Synthesis of the Drug Delivery System

Synthesis of the drug delivery system provided herein is illustrated in the synthetic schemes in the examples. The drug delivery system provided herein can be prepared using any known organic synthesis techniques and can be synthesized according to any of numerous possible synthetic routes, and thus these schemes are illustrative only and are not meant to limit other possible methods that can be used to prepare the compounds provided herein. Additionally, the steps in the schemes are for better illustration and can be changed as appropriate. The embodiments of the compounds in examples were synthesized for the purposes of research and potentially submission to regulatory agencies.

The reactions for preparing the drug delivery system of the present disclosure can be carried out in suitable solvents, which can be readily selected by one skilled in the art of organic synthesis. Suitable solvents can be substantially non-reactive with the starting materials (reactants), the intermediates, or products at the temperatures at which the reactions are carried out, e.g. temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected by one skilled in the art.

Preparation of compounds of the present disclosure can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3rd Ed., Wiley & Sons, Inc., New York (1999), in P. Kocienski, Protecting Groups, Georg Thieme Verlag, 2003, and in Peter G. M. Wuts, Greene's Protective Groups in Organic Synthesis, 5^{th} Edition, Wiley, 2014, all of which are incorporated herein by reference in its entirety.

Reactions can be monitored according to any suitable method known in the art. For example, product formation can be monitored by spectroscopic means, such as nuclear magnetic resonance spectroscopy (e.g. ¹H or ¹³C), infrared spectroscopy, spectrophotometry (e.g. UV-visible), mass spectrometry, or by chromatographic methods such as high performance liquid chromatography (HPLC), liquid chromatography-mass spectroscopy (LCMS), or thin layer chromatography (TLC). Compounds can be purified by one skilled in the art by a variety of methods, including high performance liquid chromatography (HPLC) (“Preparative LC-MS Purification: Improved Compound Specific Method Optimization” Karl F. Blom, Brian Glass, Richard Sparks, Andrew P. Combs J. Combi. Chem. 2004, 6(6), 874-883, which is incorporated herein by reference in its entirety), and normal phase silica chromatography.

The known starting materials of the present disclosure can be synthesized by using or according to the known methods in the art, or can be purchased from commercial suppliers. Unless otherwise noted, analytical grade solvents and commercially available reagents were used without further purification.

Unless otherwise specified, the reactions of the present disclosure were all done under a positive pressure of nitrogen or argon or with a drying tube in anhydrous solvents, and the reaction flasks were typically fitted with rubber septa for the introduction of substrates and reagents via syringe. Glassware was oven dried and/or heat dried.

### Method of Treatment of Disease

In a further aspect, there is provided a method of treating a disorder in a subject in need thereof, comprising administering to the subject a therapeutic effective amount of the drug delivery system or the pharmaceutical composition provided herein.

The disorder to be treated depends on the selected therapeutic agent in the drug delivery system or the pharmaceutical composition provided herein. In some embodiments, the disorder can be selected from the group consisting of inflammation, cancer, cardiovascular disease, respiratory disease, disease related to vascular endothelial growth factor (VEGF), osteoarthritis, Neovascular (Wet) Age-Related Macular Degeneration (AMD), Macular Edema Following Retinal Vein Occlusion (RVO), Diabetic Macular Edema (DME), Diabetic Retinopathy (DR), Myopic Choroidal Neovascularization (mCNV), dermatitis, psoriasis, chronic obstructive pulmonary disease, asthma.

In this context, the term “therapeutically effective amount” refers to an amount of a therapeutic agent selected in the drug delivery system provided herein or pharmaceutically acceptable salts thereof which is effective to provide “therapy” in a subject, or to “treat” disorders, diseases or conditions in a subject.

### EXAMPLES

For the purpose of illustration, the following examples are included. However, it is to be understood that these examples do not limit the present disclosure and are only meant to suggest a method of practicing the present disclosure. Persons skilled in the art will recognize that the chemical reactions described may be readily adapted to prepare a number of other compounds of the present disclosure, and alternative methods for preparing the compounds of the present disclosure are deemed to be within the scope of the present disclosure. For example, the synthesis of non-exemplified compounds according to the present disclosure may be successfully performed by modifications apparent to those skilled in the art, e.g., by appropriately protecting interfering groups, by utilizing other suitable reagents and building blocks known in the art other than those described, and/or by making routine modifications of reaction conditions. Alternatively, other reactions disclosed herein or known in the art will be recognized as having applicability for preparing other compounds of the present disclosure.

### Example 1

### Preparation of conjugate of N-(4-aminobutyl)-4-(((3R,4R)-1-(2-cyanoacetyl)-4-methylpiperidin-3-yl)(methyl)amino)-7H-pyrrolo[2,3-d]pyrimidine-7-carboxamide and HA

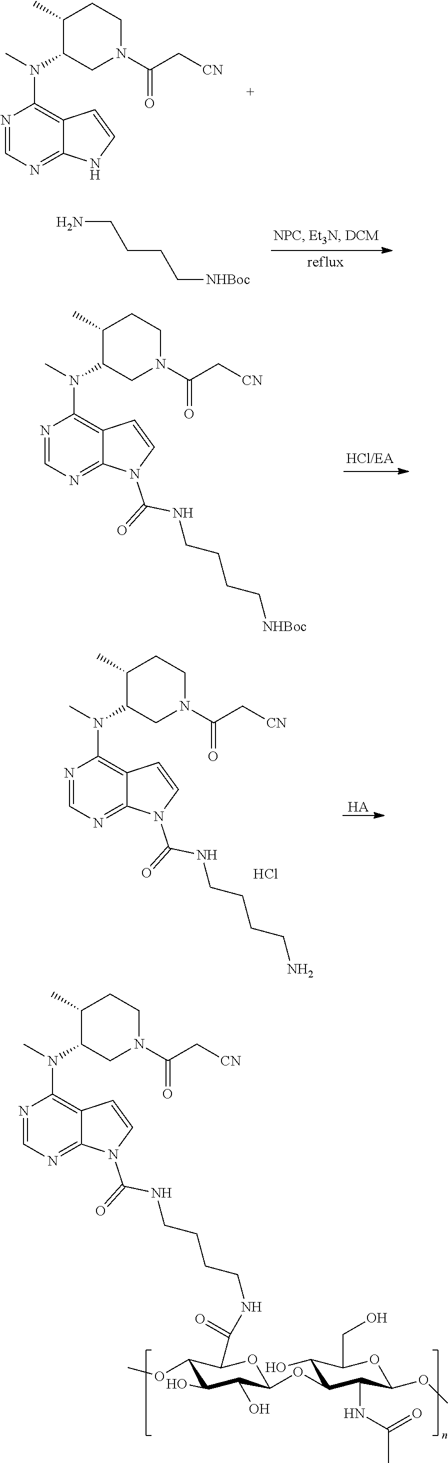

### Step 1: Preparation of tert-butyl (4-(4-(((3R,4R)-1-(2-cyanoacetyl)-4-methylpiperidin-3-yl)(methyl) amino)-7H-pyrrolo[2,3-d]pyrimidine-7-carboxamido)butyl)carbamate

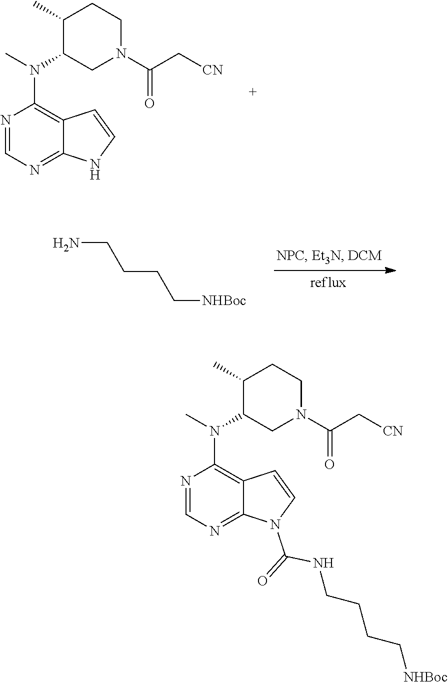

To a mixture of tofacitinib (1.5 g, 4.8 mmol, 1 eq) and bis(4-nitrophenyl) carbonate (1.61 g, 5.28 mmol, 1.1 eq) in dichloromethane (30 mL) was added triethylamine (1.2 g, 12 mmol, 2.5 eq) under N₂, the reaction mixture was heated to reflux for 3 h. Then tert-butyl (4-aminobutyl) carbamate (0.9 g, 4.82 mmol, 1 eq) was added and the resulting mixture was refluxed for 12 h. After the solvent was removed under reduced pressure, the residue was purified by silical gel chromatography to give the title product (2.4 g, yield: 95%); MS (m/z): [M+H]+ calcd for C₂₆H₃₈N₈O₄, 527.30; found, 527.2.

### Step 2: Preparation of N-(4-aminobutyl)-4-(((3R,4R)-1-(2-cyanoacetyl)-4-methyl piperidin-3-yl)(methyl)amino)-7H-pyrrolo[2,3-d]pyrimidine-7-carboxamide hydrochloride

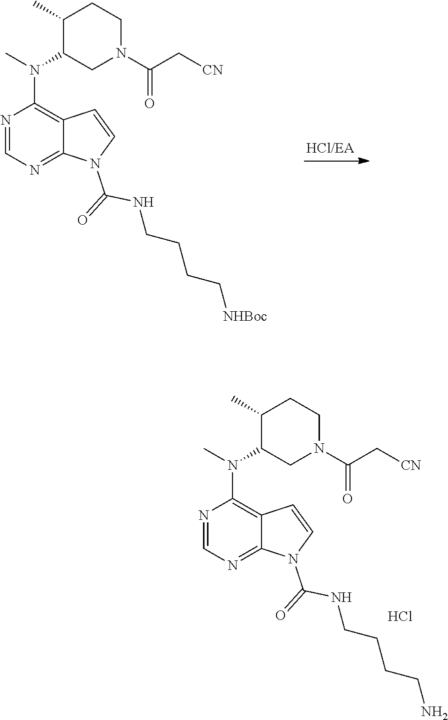

To a solution of tert-butyl (4-(4-(((3R,4R)-1-(2-cyanoacetyl)-4-methylpiperidin-3-yl)(methyl)amino)-7H-pyrrolo[2,3-d]pyrimidine-7-carboxamido)butyl)carbamate (2.4 g, 4.56 mmol, 1 eq) in ethyl acetate (24 mL) was added 4M HCl in ethyl acetate solution (9.6 mL, 38.4 mmol) dropwisely under N₂ at 0° C. The resulting reaction mixture was stirred at 0° C. for 30 min and then stirred at room temperature for 2 days. The solvent was removed under reduced pressure, the resulting solid was stirred in ethyl acetate (24 mL) for 0.5 h, then filtered to give the desired product as HCl salt (2.1 g, yield: 100%); MS (m/z): [M+H]+ calcd for C₂₁H₃₀N₈O₂, 427.25; found, 427.2. ¹H-NMR (400 MHz, D₂O) δ ppm 8.38 (d, J=6.7 Hz, 1H), 7.82 (d, J=3.9 Hz 1H), 6.90 (s, 1H), 4.72-4.53 (m, 1H), 4.16-3.26 (m, 11H), 3.04 (d, J=6.4 Hz 2H), 2.66-2.46 (m, 1H), 2.03-1.90 (m, 1H), 1.86-1.69 (m, 5H), 1.12 (d, J=7.1 Hz, 3H).

### Step 3: Preparation of conjugate of N-(4-aminobutyl)-4-(((3R,4R)-1-(2-cyanoacetyl)-4-methylpiperidin-3-yl)(methyl)amino)-7H-pyrrolo[2,3-d]pyrimidine-7-carboxamide and HA

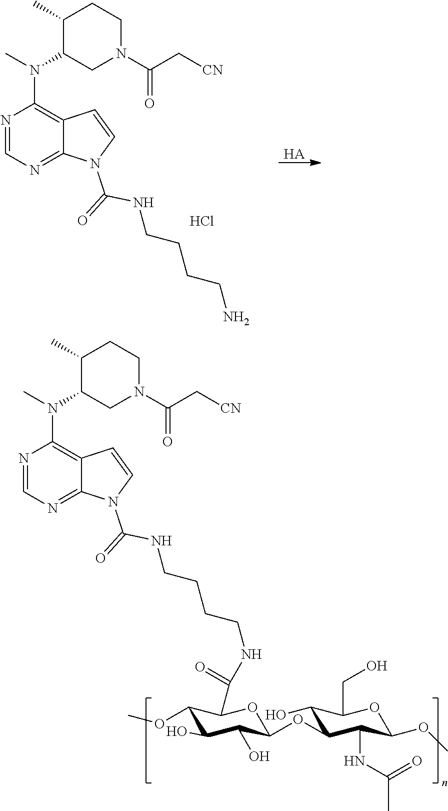

To a solution of sodium hyaluronate (MW 50 KDa, 0.161 g, 0.432 mmol, 1 eq) in Acetonitrile (22 mL) and H₂O (35 mL), 4-methylmorpholine (0.066 g, 0.65 mmol, 1.5 eq) and 2-chloro-4,6-dimethoxy-1,3,5-triazine (0.076 g, 0.432 mmol, 1 eq) were added at 0° C. The resulting reaction mixture was stirred at 0° C. for 30 min and then stirred at room temperature for 1 h.

N-(4-aminobutyl)-4-((3R,4R)-1-(2-cyanoacetyl)-4-methylpiperidin-3-yl)(methyl)amino)-7H-pyrrolo[2,3-d]pyrimidine-7-carboxamide hydrochloride (0.2 g, 0.432 mmol, leg) was added to the reaction mixture and then the pH of the reaction mixture was adjusted to 6.5 to 7 with 4-methylmorpholine. The resulting reaction mixture was stirred 3 days at room temperature.

NaCl (257 mg, 10 eq) in H₂O (2 mL) was added to the above reaction mixture and stirred for 0.5 h. Then acetone (350 mL) was added dropwise to the above mixture, while the precipitate was formed. The mixture was filtered and the cake was washed with acetone (10 mL*3). The wet cake was dissolved in Acetonitrile (20 mL) and H₂O (40 mL) and then dialysed with 3.5 kDa MW cutoff film against deionized water for 3 times, then lyophilized to afford the title compound (0.15 g, yield: 43.2%, DSR (Drug substitution rate)=17%). ¹H-NMR (400 MHz, D₂O/d-DMSO=3:1) δ ppm 8.40-7.90 (m, 0.17H), 7.75-7.20 (m, 0.17H), 6.95-6.25 (m, 0.17H), 4.70-4.20 (m, 2.47H), 4.00-3.23 (m, 11.91H), 2.55-2.30 (m. 0.34H), 1.99 (d, J=19.7 Hz, 3H), 1.30 (t, J=6.8 Hz, 0.17H), 1.20-1.10 (m, 0.51H).

With the Step 3, reaction of sodium hyaluronate (MW 500 KDa) provided corresponding product (0.16 g, yield: 46%, DSR=22%). NMR

With the Step 3, reaction of sodium hyaluronate (MW 2000 KDa) provided corresponding product (0.15 g, yield: 43.2%, DSR=17%). NMR

### Example 2

### Preparation of conjugate N-(4-(aminomethyl)phenyl)-4-(((3R,4R)-1-(2-cyanoacetyl)-4-methylpiperidin-3-yl)(methyl)amino)-7H-pyrrolo[2,3-d]pyrimidine-7-carboxamide and HA

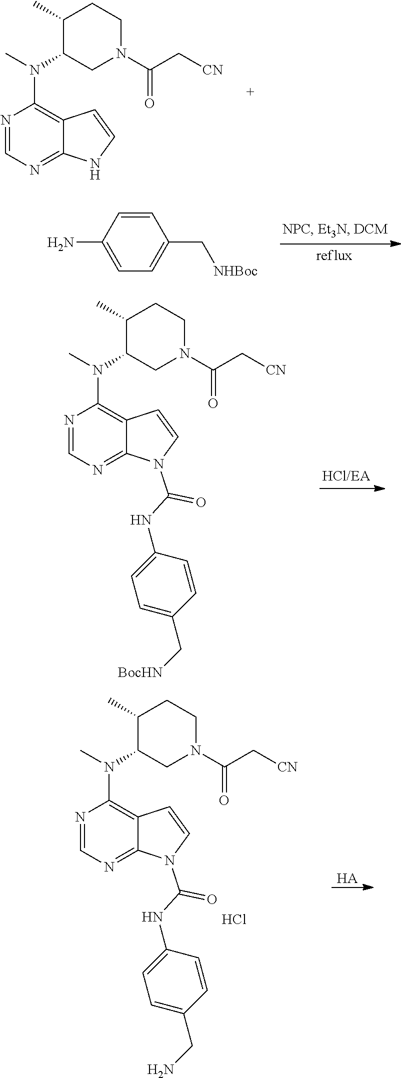

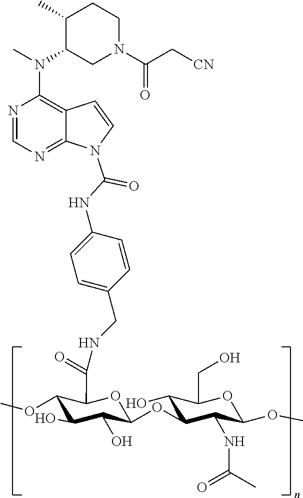

### Step 1: Preparation of tert-butyl(4-(4-(((3R,4R)-1-(2-cyanoacetyl)-4-methylpiperidin-3-yl)(methyl)amino)-7H-pyrrolo[2,3-d]pyrimidine-7-carboxamido)benzyl)carbamate

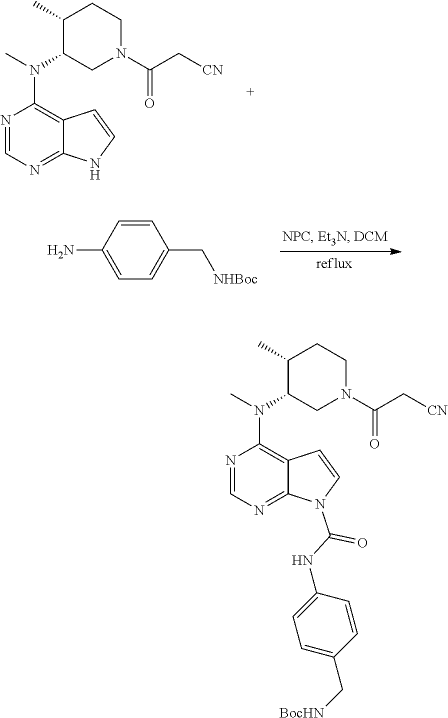 [SMILES: C[C@@H]1CCN(C(=O)CC#N)C[C@@H]1N(C)c1ncnc2[nH]ccc12 ; CC(C)(C)OC(=O)NCc1ccc(N)cc1 ; C[C@@H]1CCN(C(=O)CC#N)C[C@@H]1N(C)c1ncnc2c1ccn2C(=O)Nc1ccc(CNC(=O)OC(C)(C)C)cc1]

By following Step 1 in Example 1, tofacitinib (1.5 g, 4.8 mmol, 1 eq), bis(4-nitrophenyl) carbonate (1.61 g, 5.28 mmol, 1.1 eq), and tert-Butyl (4-aminobutyl) carbamate (1.07 g, 4.8 mmol, 1 eq) gave the title product (1.8 g, yield: 67%); MS (m/z): [M+H]+ calcd for C₂₉H₃₆N₈O₄, 562.29; found, 562.2.

### Step 2: Preparation of N-(4-(aminomethyl)phenyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride

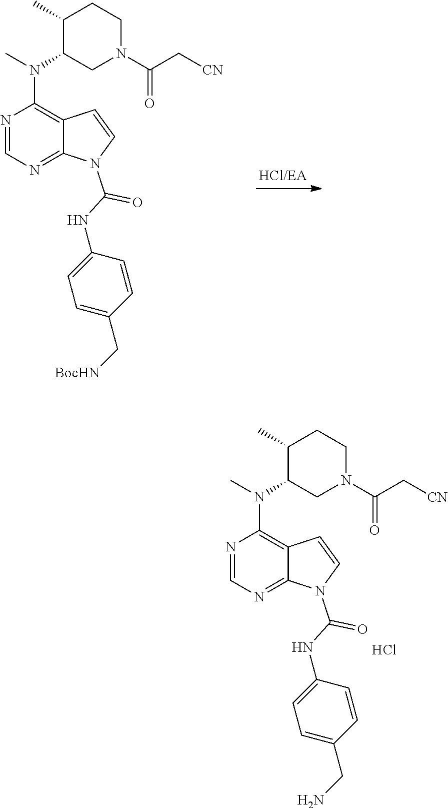 [SMILES: C[C@@H]1CCN(C(=O)CC#N)C[C@@H]1N(C)c1ncnc2c1ccn2C(=O)Nc1ccc(CNC(=O)OC(C)(C)C)cc1 ; C[C@@H]1CCN(C(=O)CC#N)C[C@@H]1N(C)c1ncnc2c1ccn2C(=O)Nc1ccc(CN)cc1 ; Cl]

By following Step 2 in Example 1, tert-butyl (4-(4-(((3R,4R)-1-(2-cyanoacetyl)-4-methyl piperidin-3-yl)(methyl)amino)-7H-pyrrolo[2,3-d]pyrimidine-7-carboxamido) benzyl) carbamate (1.5 g, 2.675 mmol, 1 eq) gave the desired product as HCl salt (1.33 g, yield: 100%); MS (m/z): [M+H]+ calcd for C₂₄H₂₈N₈O₂, 461.23; found, 461.2. ¹H-NMR (400 MHz, D₂O) δ ppm) δ 8.30-8.08 (m, 1H), 7.7-7.3 (m, 5H), 6.80-6.50 (m, 1H), 4.55 (s, 2H), 4.19 (s, 2H), 4.09-3.88 (m, 3H), 3.6-3.19 (m, 5H), 2.44 (br, 1H), 2.0-1.5 (m, 2H), 1.25-1.0 (m, 3H).

### Step 3: Preparation of conjugate N-(4-(aminomethyl)phenyl)-4-(((3R,4R)-1-(2-cyanoacetyl)-4-methylpiperidin-3-yl)(methyl)amino)-7H-pyrrolo[2,3-d]pyrimidine-7-carboxamide and HA

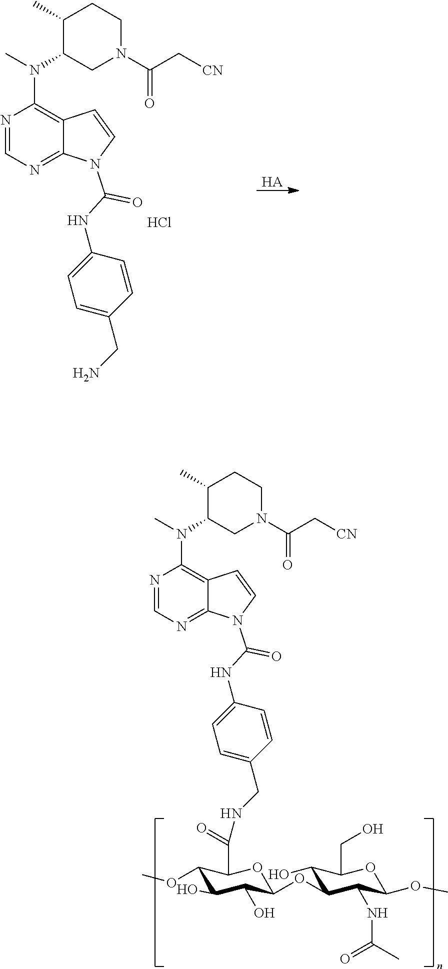

By following Step 3 of Example 1, sodium hyaluronate (MW 50 KDa, 0.163 g, 0.403 mmol, 1 eq) and N-(4-(aminomethyl)phenyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride (0.2 g, 0.403 mmol, 1 eq) reaction mixture afforded the title compound (0.18 g, yield: 55%, DSR=38%); ¹H-NMR (400 MHz, D₂O/d-DMSO=3:1) δ ppm 9.0-7.0 (m, 2.7H), 4.75-4.4 (m, 5.5H), 4.4-3.0 (m, 11H), 2.7-2.45 (m, 0.38H), 2.15 (s, 3H), 1.9-1.4 (m, 0.74H), 1.4-0.9 (m, 1.14H).

With Step 3 of Example 1, reaction of sodium hyaluronate (MW 500 KDa) provided corresponding product (0.15 g, yield: 44%, DSR=21.4%).

With Step 3 of Example 1, reaction of sodium hyaluronate (MW 2000 KDa) provided corresponding product (0.18 g, yield: 53%, DSR=20.7%)

### Example 3

### Preparation of conjugate of tert-butyl(4-(4-(((3R,4R)-1-(2-cyanoacetyl)-4-methylpiperidin-3-yl)(methyl)amino)-7H-pyrrolo[2,3-d]pyrimidine-7-carboxamido)phenethyl)carbamate and HA

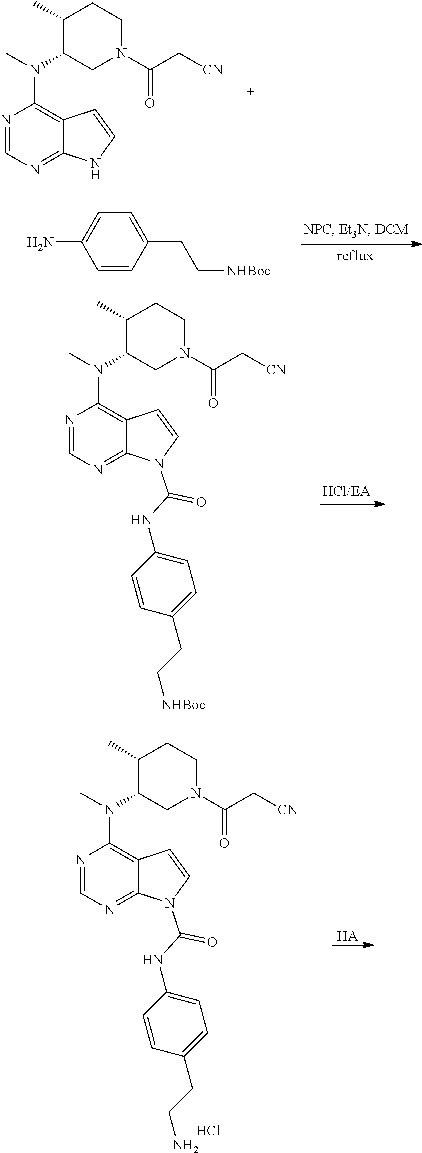

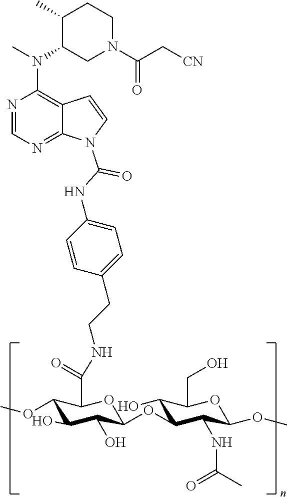

### Step 1: Preparation of tert-butyl (4-(4-(((3R,4R)-1-(2-cyanoacetyl)-4-methylpiperidin-3-yl)(methyl)amino)-7H-pyrrolo[2,3-d]pyrimidine-7-carboxamido)phenethyl)carbamate

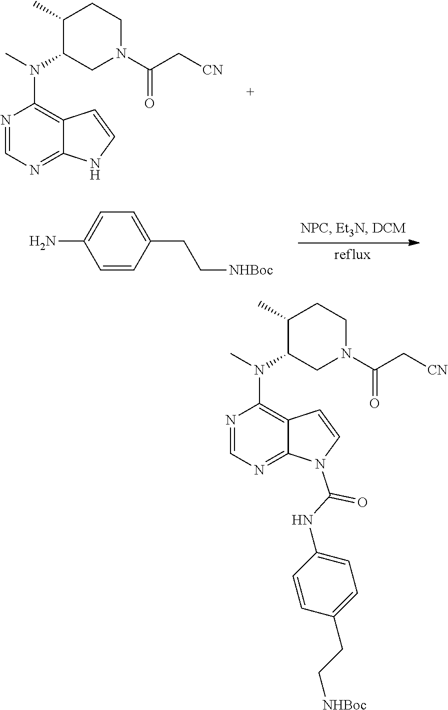

By following Step 1 of Example 1, tofacitinib (1.5 g, 4.8 mmol, 1 eq) and tert-Butyl (4-aminophenethyl) carbamate (1.14 g, 4.8 mmol, 1 eq) gave the title product (2.2 g, yield: 79.7%); MS (m/z): [M+H]+ calcd for C₃₀H₃₈N₈O₄, 575.30; found, 575.2.

### Step 2: Preparation of N-(4-(2-aminoethyl)phenyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride

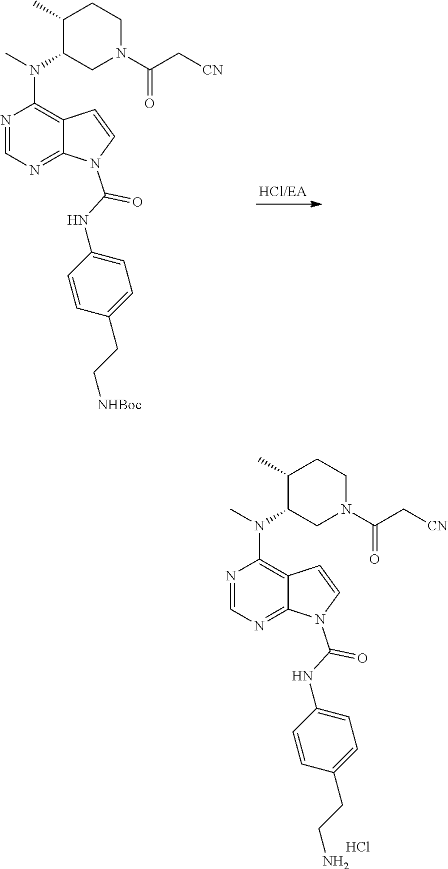 [SMILES: C[C@@H]1CCN(C(=O)CC#N)C[C@@H]1N(C)c1ncnc2c1ccn2C(=O)Nc1ccc(CCNC(=O)OC(C)(C)C)cc1 ; C[C@@H]1CCN(C(=O)CC#N)C[C@@H]1N(C)c1ncnc2c1ccn2C(=O)Nc1ccc(CCN)cc1 ; Cl]

By following Step 2 of Example 1, tert-butyl (4-(4-(((3R,4R)-1-(2-cyanoacetyl)-4-methylpiperidin-3-yl)(methyl)amino)-7H-pyrrolo[2,3-d]pyrimidine-7-carboxamido)phenethyl)carbamate (1.5 g, 2.61 mmol, 1 eq) gave the desired product as HCl salt (1.33 g, yield: 100%); MS (m/z): [M+H]-+ calcd for C₂₅H₃₀N₈O₂, 475.25; found, 475.2. ¹H-NMR (400 MHz, D₂O) δ ppm 8.31-8.01 (m, 1H), 7.96-7.09 (m, 5H), 6.98-6.54 (m, 1H), 4.50 (dd, 2H), 4.10-3.42 (m, 5H), 3.25 (d, J=21.8 Hz, 5H), 3.02 (t, 2H), 2.43 (d, J=5.7 Hz, 1H), 1.83 (dd, J=72.1, 15.3 Hz, 2H), 1.13 (dd, J=18.5, 13.3 Hz, 3H).

### Step 3: Preparation of conjugate of tert-butyl(4-(4-(((3R,4R)-1-(2-cyanoacetyl)-4-methylpiperidin-3-yl)(methyl)amino)-7H-pyrrolo[2,3-d]pyrimidine-7-carboxamido)phenethyl)carbamate and HA

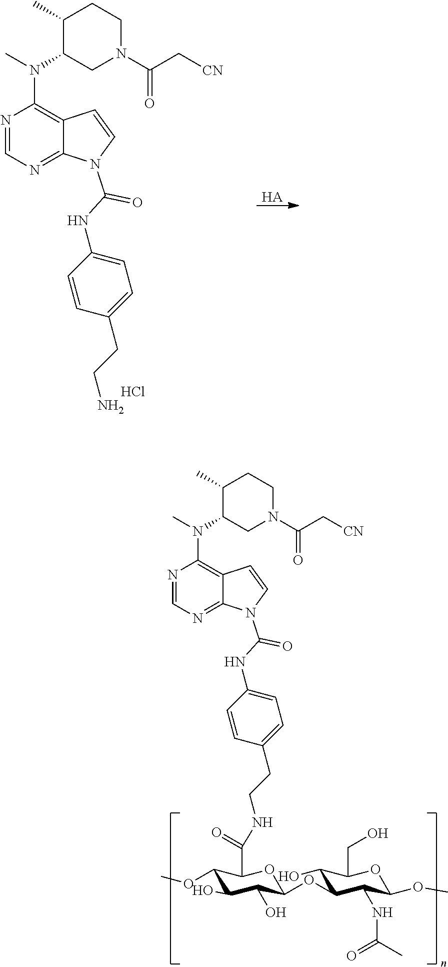

By following Step 3 of Example 1, sodium hyaluronate (MW 50 KDa, 0.157 g, 0.39 mmol, 1 eq) and N-(4-(2-aminoethyl)phenyl)-4-(((3R,4R)-1-(2-cyanoacetyl)-4-methylpiperidin-3-yl) (methyl)amino)-7H-pyrrolo[2,3-d]pyrimidine-7-carboxamide hydrochloride (0.2 g, 0.39 mmol, 1 eq) afforded the title compound (0.19 g, yield: 33.4%, DSR=19%). ¹H-NMR (400 MHz, D₂O/DMSO=3:1) δ ppm 8.5-8.25 (m, 0.09H), 7.90-6.72 (m, 1.23H), 4.75-4.25 (m, 1.98H), 4.02-3.17 (m, 12H), 3.10-2.75 (m, 0.76H), 2.54 (br, 0.19H), 2.04 (s, 3H), 1.60-1.25 (m, 0.38H), 1.18-0.98 (m, 0.57H).

With Step 3 of Example 1, reaction of sodium hyaluronate (MW 500 KDa) provided corresponding product (0.147 g, yield: 44%, DSR=16%),

With Step 3 of Example 1, reaction of sodium hyaluronate (MW 2000 KDa) provided corresponding product (0.175 g, yield: 53%, DSR=22.9%).

### Example 4

### Preparation of conjugate of N-(2-(2-(2-aminoethoxy) ethoxy) ethyl)-4-(((3R, 4R)-1-(2-cyanoacetyl)-4-methylpiperidin-3-yl)(methyl)amino)-7H-pyrrolo [2,3-d]pyrimidine-7-carboxamide and HA

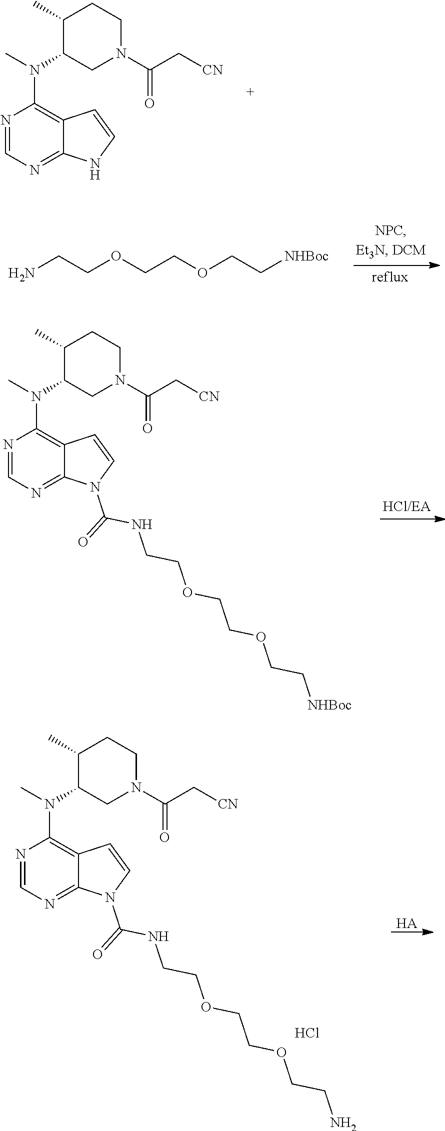 [SMILES: C[C@@H]1CCN(C(=O)CC#N)C[C@@H]1N(C)c1ncnc2[nH]ccc12 ; CC(C)(C)OC(=O)NCCOCCOCCN ; C[C@@H]1CCN(C(=O)CC#N)C[C@@H]1N(C)c1ncnc2c1ccn2C(=O)NCCOCCOCCNC(=O)OC(C)(C)C ; C[C@@H]1CCN(C(=O)CC#N)C[C@@H]1N(C)c1ncnc2c1ccn2C(=O)NCCOCCOCCN ; Cl ; "HA"]

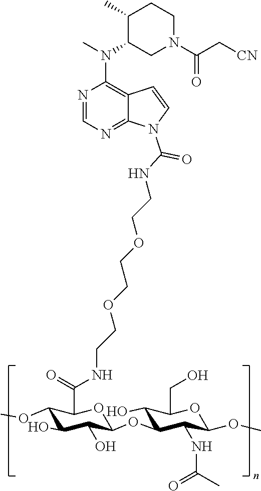

### Step 1: Preparation of tert-butyl (2-(2-(2-(4-(((3R,4R)-1-(2-cyanoacetyl)-4-methylpiperidin-3-yl)(methyl)amino)-7H-pyrrolo[2,3-d]pyrimidine-7-carboxamido)ethoxy)ethoxy)ethyl)carbamate

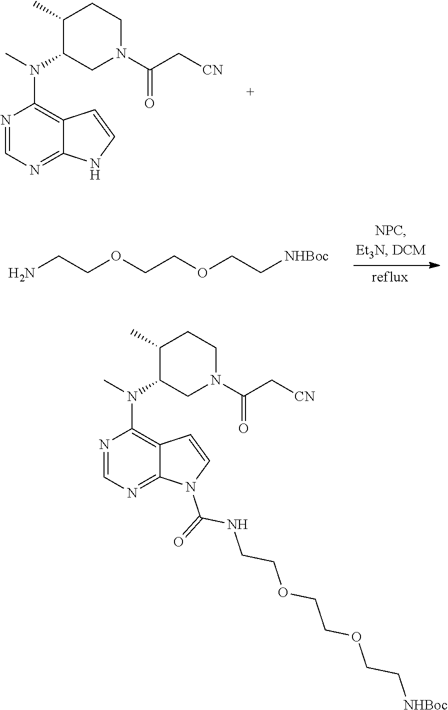 [SMILES: C[C@@H]1CCN(C(=O)CC#N)C[C@@H]1N(C)c1ncnc2[nH]ccc12 ; CC(C)(C)OC(=O)NCCOCCOCCN ; C[C@@H]1CCN(C(=O)CC#N)C[C@@H]1N(C)c1ncnc2c1ccn2C(=O)NCCOCCOCCNC(=O)OC(C)(C)C]

By following Step 1 of Example 1, tofacitinib (3 g, 9.6 mmol, 1 eq) tert-butyl (2-(2-(2-aminoethoxy) ethoxy) ethyl) carbamate (2.38 g, 9.6 mmol, 1 eq) gave the title product (3 g, yield: 54%); MS (m/z): [M+H]+ calcd for C₂₈H₄₂N₈O₆, 587.32; found, 587.2.

### Step 2: Preparation of N-(2-(2-(2-aminoethoxy)ethoxy)ethyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride

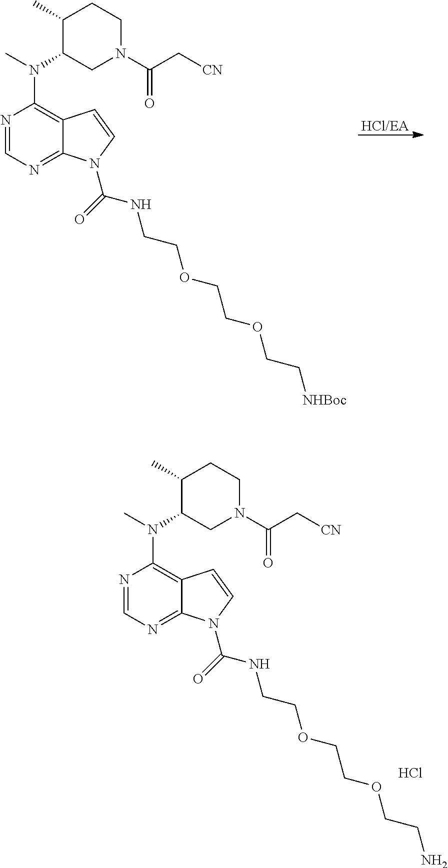 [SMILES: C[C@@H]1CCN(C(=O)CC#N)C[C@@H]1N(C)c1ncnc2c1ccn2C(=O)NCCOCCOCCNC(=O)OC(C)(C)C ; "HCl/EA" ; C[C@@H]1CCN(C(=O)CC#N)C[C@@H]1N(C)c1ncnc2c1ccn2C(=O)NCCOCCOCCN ; Cl]

By following Step 2 of Example 1, tert-butyl (2-(2-(2-(4-(((3R,4R)-1-(2-cyanoacetyl)-4-methylpiperidin-3-yl)(methyl)amino)-7H-pyrrolo[2,3-d]pyrimidine-7-carboxamido)ethoxy)ethoxy) ethyl)carbamate (1.5 g, 2.56 mmol, 1 eq) gave the desired product as HCl salt (1.34 g, yield: 100%); MS (m/z): [M+H]+ calcd for C₂₃H₃₄N₈O₄, 487.32; found, 487.2. ¹H-NMR (400 MHz, D₂O) δ ppm 8.40 (br, 1H), 7.83 (s, 1H), 6.91 (s, 1H), 4.7-4.5 (m, 1H), 4.06-3.92 (m, 3H), 3.83-3.36 (m, 16H), 3.17 (s, 2H), 2.56 (s, 1H), 2.00-1.72 (m, 2H), 1.14 (dd, J=16.1, 7.0 Hz, 3H).

### Step 3: Preparation of conjugate of N-(2-(2-(2-aminoethoxy) ethoxy) ethyl)-4-(((3R, 4R)-1-(2-cyanoacetyl)-4-methylpiperidin-3-yl)(methyl)amino)-7H-pyrrolo [2,3-d]pyrimidine-7-carboxamide and HA

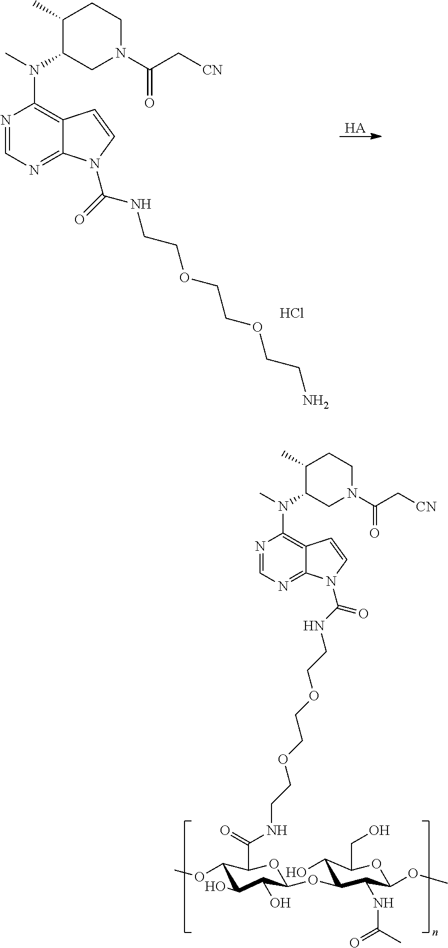

By following Step 3 of Example 1, sodium hyaluronate (MW 50 KDa, 0.153 g, 0.38 mmol, 1 eq) and N-(2-(2-(2-aminoethoxy)ethoxy)ethyl)-4-(((3R,4R)-1-(2-cyanoacetyl)-4-meth ylpiperidin-3-yl)(methyl)amino)-7H-pyrrolo[2,3-d]pyrimidine-7-carboxamide hydro chloride (0.2 g, 0.38 mmol, 1 eq) afforded the title compound (0.2 g, yield: 33%, DSR=36.3%). ¹H-NMR (400 MHz, D₂O/d-DMSO=3:1) δ ppm 8.52-7.93 (m, 0.37H), 7.82-7.20 (m, 0.33H), 6.99-6.26 (m, 0.39H), 4.75-3.8 (m, 5.09H), 3.8-2.75 (m, 14.93H), 2.5-2.25 (m, 0.36H), 2.05 (s, 3H), 1.82-1.30 (m, 0.73H), 1.2-0.8 (m, 1.09H).

With Step 3 of Example 1, reaction of sodium hyaluronate (MW 500 KDa) provided corresponding product (0.186 g, yield: 56.5%, DSR=32%)

With Step 3 of Example 1, reaction of sodium hyaluronate (MW 2000 KDa) provided corresponding product (0.198 g, yield: 60.2%, DSR=26.6%).

### Example 5

### Preparation of conjugate of N-(2-(2-(2-(2-aminoethoxy)ethoxy)ethoxy)ethyl)-4-(((3R,4R)-1-(2-cyanoacetyl)-4-methylpiperidin-3-yl)(methyl)amino)-7H-pyrrolo[2,3-d]pyrimidine-7-carboxamide and HA

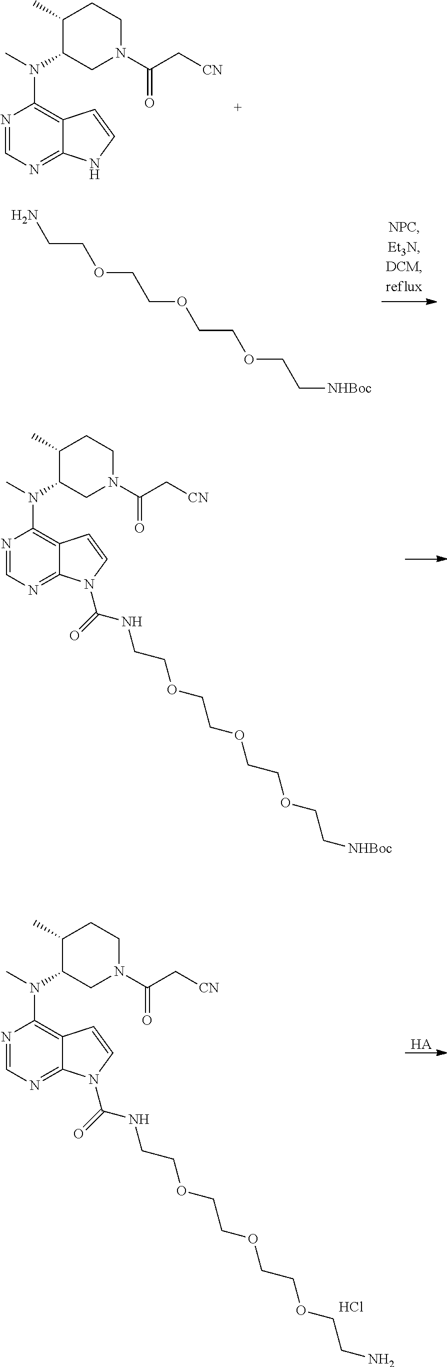

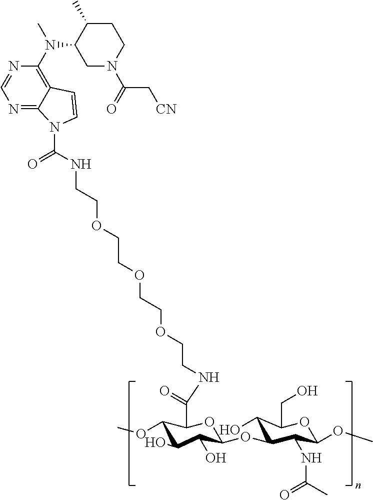

### Step 1: Preparation of tert-butyl (1-(4-(((3R,4R)-1-(2-cyanoacetyl)-4-methylpiperidin-3-yl)(methylamino)-7H-pyrrolo[2,3-d]pyrimidin-7-yl)-1-oxo-5,8,11-trioxa-2-azatridecan-13-yl)carbamate

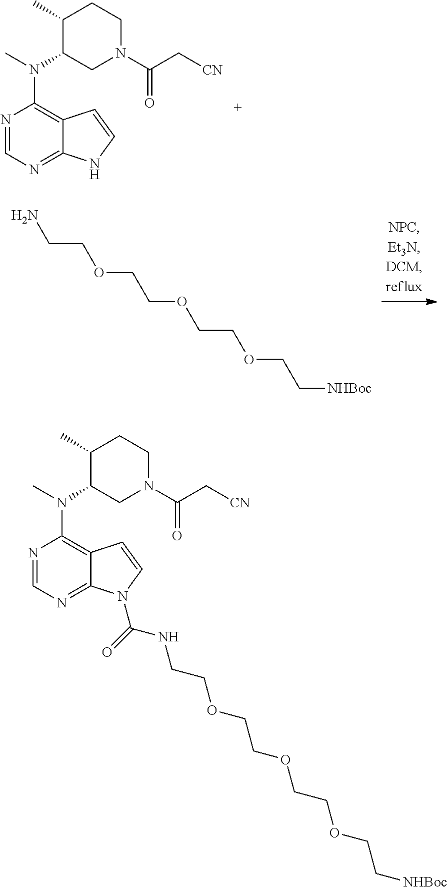

By following Step 1 of Example 1, tofacitinib (1.5 g, 4.8 mmol, 1 eq) and tert-butyl (2-(2-(2-(2-aminoethoxy)ethoxy)ethoxy)ethyl)carbamate (1.41 g, 4.8 mmol, 1 eq) gave the title product (1.8 g, yield: 60%); MS (m/z): [M+H]+ calcd for C₃₀H₄₆N₈O₇, 631.35; found, 631.2.

### Step 2: Preparation of N-(2-(2-(2-(2-aminoethoxy)ethoxy)ethoxy)ethyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride

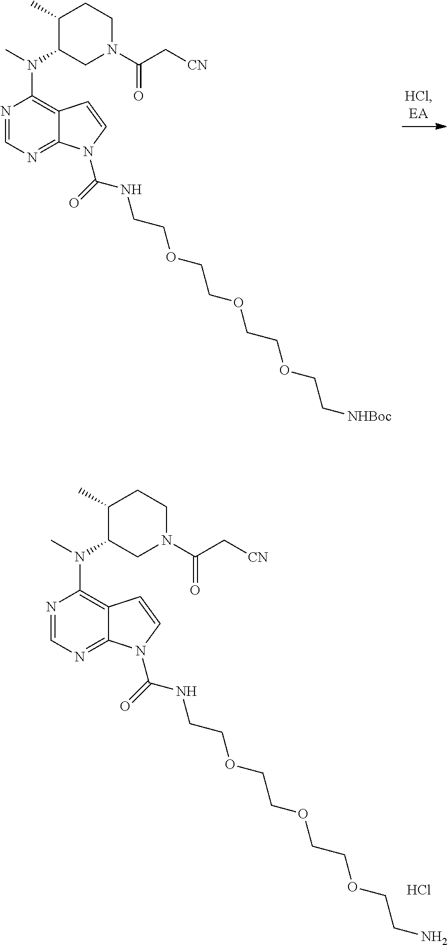 [SMILES: C[C@@H]1CCN(C(=O)CC#N)C[C@@H]1N(C)c1ncnc2c1ccn2C(=O)NCCOCCOCCOCCNC(=O)OC(C)(C)C ; C[C@@H]1CCN(C(=O)CC#N)C[C@@H]1N(C)c1ncnc2c1ccn2C(=O)NCCOCCOCCOCCN ; Cl]

By following Step 2 of Example 1, tert-butyl (1-(4-(((3R,4R)-1-(2-cyanoacetyl)-4-methylpiperidin-3-yl)(methylamino)-7H-pyrrolo[2,3-d]pyrimidin-7-yl)-1-oxo-5,8,11-trioxa-2-azatridecan-13-yl)carbamate (1.5 g, 2.38 mmol 1 eq) gave the desired product as HCl salt (1 g, yield: 80%); MS (m/z): [M+H]+ calcd for C₂₅H₃₈N₈O₅, 531.30; found, 531.2. ¹H-NMR (400 MHz, DMSO) δ ppm 9.74 (s, 1H), 8.34 (d, J=7.0 Hz, 1H), 8.01 (s, 1H), 7.69 (s, 1H), 6.86 (s, 1H), 4.85 (d, J=3.1 Hz, 1H), 4.35-4.0 (m, 10H), 3.7-3.96 (m, 3H), 3.65-3.5 (m, 10H), 2.93 (d, J=4.3 Hz, 2H), 2.38 (s, 1H), 1.89-1.68 (m, 1H), 1.59 (s, 1H), 1.01 (d, J=4.8 Hz, 3H).

### Step 3: Preparation of conjugate of N-(2-(2-(2-(2-aminoethoxy)ethoxy)ethoxy)ethyl)-4-(((3R,4R)-1-(2-cyanoacetyl)-4-methylpiperidin-3-yl)(methyl)amino)-7H-pyrrolo[2,3-d]pyrimidine-7-carboxamide and HA

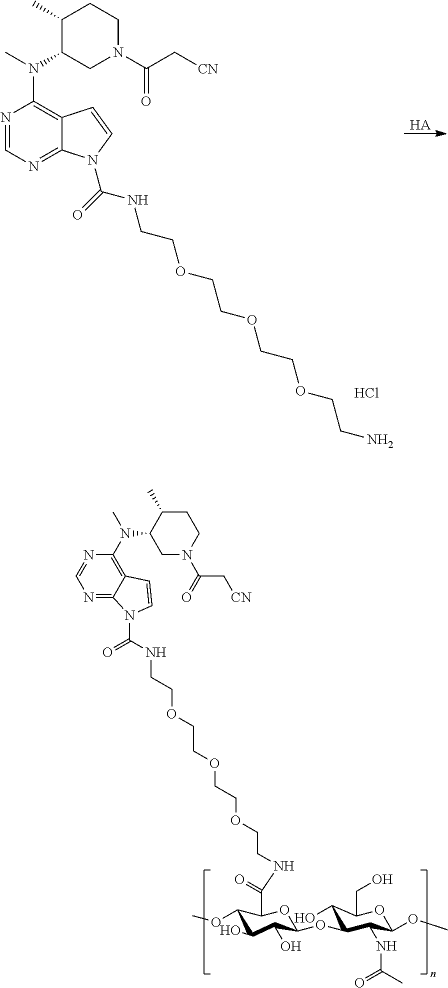

By following Step 3 of Example 1, sodium hyaluronate (MW 50 KDa, 0.141 g, 0.35 mmol, 1 eq) and N-(2-(2-(2-(2-aminoethoxy)ethoxy)ethoxy)ethyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride (0.2 g, 0.35 mmol, leg) afforded the title compound (0.2 g, yield: 62.7%, DSR=24%); ¹H-NM/R (400 MHz, D₂O-DMSO=3:1) δ ppm 8.7-8.2 (m, 0.24H), 8.1-7.3 (m, 0.24H), 7.2-6.6 (m, 0.24H), 4.7-4.35 (m, 1H), 4.3-3.1 (m, 17H), 2.7-2.5 (br, 0.24H), 2.14 (br, 3H), 1.93 (br, 0.24H), 1.46 (br, 0.24H), 1.24 (br, 0.72H).

With Step 3 of Example 1, reaction of sodium hyaluronate (MW 500 KDa) provided corresponding product (0.186 g, yield: 58.4%, DSR=26%)

With Step 3 of Example 1, reaction of sodium hyaluronate (MW 2000 KDa) provided corresponding product (0.198 g, yield: 62.2%, DSR=28%).

### Example 6

### Preparation of conjugate of 4-(((3R,4R)-1-(2-cyanoacetyl)-4-methylpiperidin-3-yl)(methyl)amino)-7H-pyrrolo[2,3-d]pyrimidine-7-carbohydrazide and HA

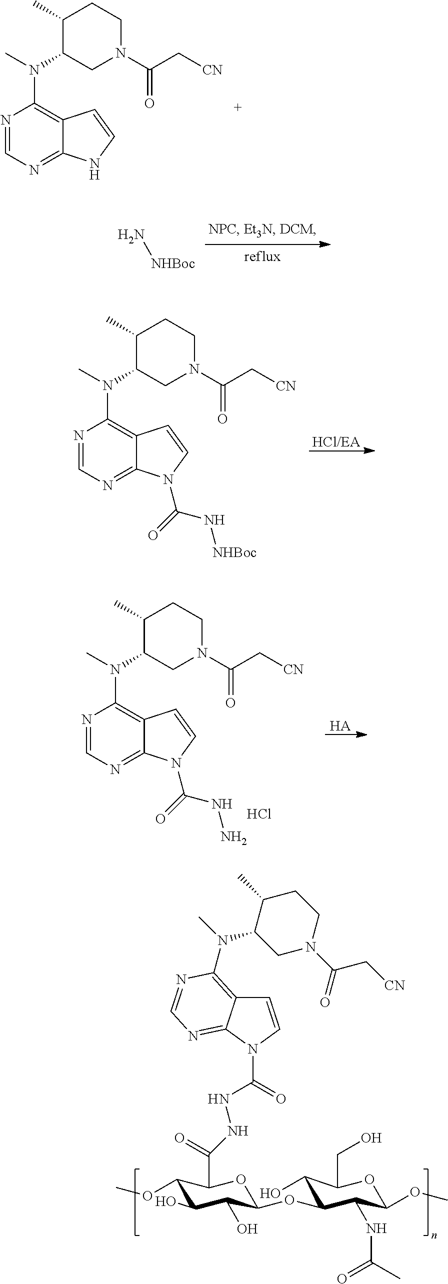

### Step 1: Preparation of tert-butyl 2-(4-(((3R,4R)-1-(2-cyanoacetyl)-4-methylpiperidin-3-yl)(methyl)amino)-7H-pyrrolo[2,3-d]pyrimidine-7-carbonyl)hydrazine-1-carboxylate

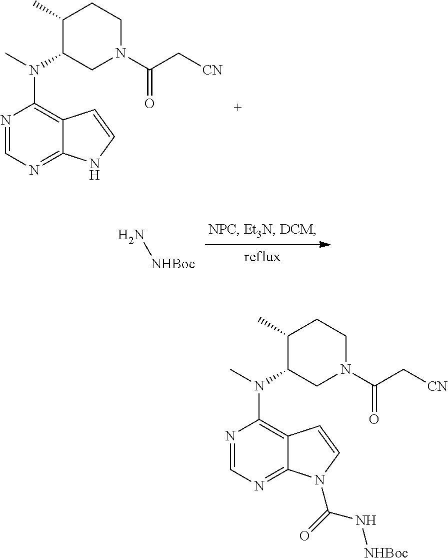 [SMILES: C[C@@H]1CCN(C(=O)CC#N)C[C@@H]1N(C)c1ncnc2[nH]ccc12 ; CC(C)(C)OC(=O)NN ; C[C@@H]1CCN(C(=O)CC#N)C[C@@H]1N(C)c1ncnc2c1ccn2C(=O)NNC(=O)OC(C)(C)C]

By following Step 1 of Example 1, tofacitinib (2 g, 6.4 mmol, 1 eq) and tert-butyl hydrazinecarboxylate (0.845 g, 6.4 mmol, 1 eq) gave the title product (2.23 g, yield: 75%); MS (m/z): [M+H]+ calcd for C₂₂H₃₀N₈O₄, 471.24; found, 471.2.

### Step 2: Preparation of 4-(((3R,4R)-1-(2-cyanoacetyl)-4-methylpiperidin-3-yl)(methyl)amino)-7H-pyrrolo[2,3-d]pyrimidine-7-carbohydrazide hydrochloride

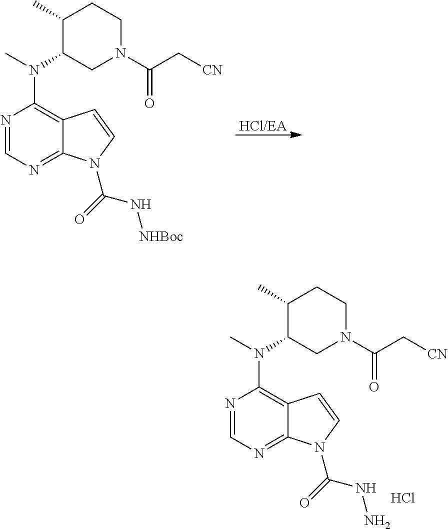 [SMILES: C[C@@H]1CCN(C(=O)CC#N)C[C@@H]1N(C)c1ncnc2c1ccn2C(=O)NNC(=O)OC(C)(C)C ; "HCl/EA" ; C[C@@H]1CCN(C(=O)CC#N)C[C@@H]1N(C)c1ncnc2c1ccn2C(=O)NN ; Cl]

By following Step 2 of Example 1, tert-butyl2-(4-(((3R,4R)-1-(2-cyanoacetyl)-4-methyl piperidin-3-yl)(methyl)amino)-7H-pyrrolo[2,3-d]pyrimidine-7-carbonyl)hydrazine-1-carboxylate (2 g, 4.25 mmol, 1 eq) gave the desired product as HCl salt (1.5 g, yield: 85%); MS (m/z): [M+H]+ calcd for C₁₇H₂₂N₈O₂, 371.19; found, 371.1. ¹H-NMR (400 MHz, CD₃OD) δ ppm 8.52 (s, 1H), 7.89 (d, J=3.1 Hz, 1H), 7.13 (d, J=3.8 Hz, 1H), 4.83 (s, 1H), 4.22-3.32 (m, 9H), 2.55 (br, 1H), 2.05-1.99 (m, 1H), 1.90-1.67 (m, 1H), 1.16 (d, J=7.0 Hz, 3H).

### Step 3: Preparation of conjugate of 4-(((3R,4R)-1-(2-cyanoacetyl)-4-methylpiperidin-3-yl)(methyl)amino)-7H-pyrrolo[2,3-d]pyrimidine-7-carbohydrazide and HA

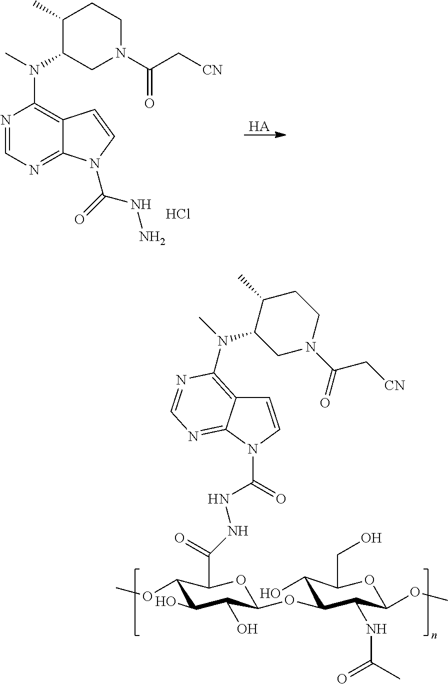

By following Step 3 of Example 1, sodium hyaluronate (MW 50 KDa, 0.202 g, 0.5 mmol, 1 eq) and 4-(((3R,4R)-1-(2-cyanoacetyl)-4-methylpiperidin-3-yl)(methyl)amino)-7H-pyrrolo[2,3-d]pyrimidine-7-carbohydrazide hydrochlori de (0.2 g, 0.5 mmol, 1 eq) afforded the title compound (0.15 g, yield: 43.2%, DSR=32%); ¹H-NMR (400 MHz, D₂O-DMSO=5:1): δ ppm 8.4-7.8 (m, 0.32H), 7.8-7.2 (m, 0.32H), 7.0-6.0 (m, 0.32H), 4.75-4.4 (m, 2.1H), 4.26-3.19 (m, 11.1H), 2.45 (br, 0.32H), 2.05 (br, 3H), 1.85-1.3 (m, 0.65H), 1.18 (br, 0.95H).

With Step 3 of Example 1, reaction of sodium hyaluronate (MW 500 KDa) provided corresponding product (0.18 g, yield: 48.5%, DSR=35%).

With Step 3 of Example 1, reaction of sodium hyaluronate (MW 2000 KDa) provided corresponding product (0.19 g, yield: 51.2%, DSR=30%).

### Example 7

### Preparation of conjugate of N-(2-aminoethyl)-4-(((3R,4R)-1-(2-cyanoacetyl)-4-methylpiperidin-3-yl)(methyl)amino)-7H-pyrrolo[2,3-d]pyrimidine-7-carboxamide and HA

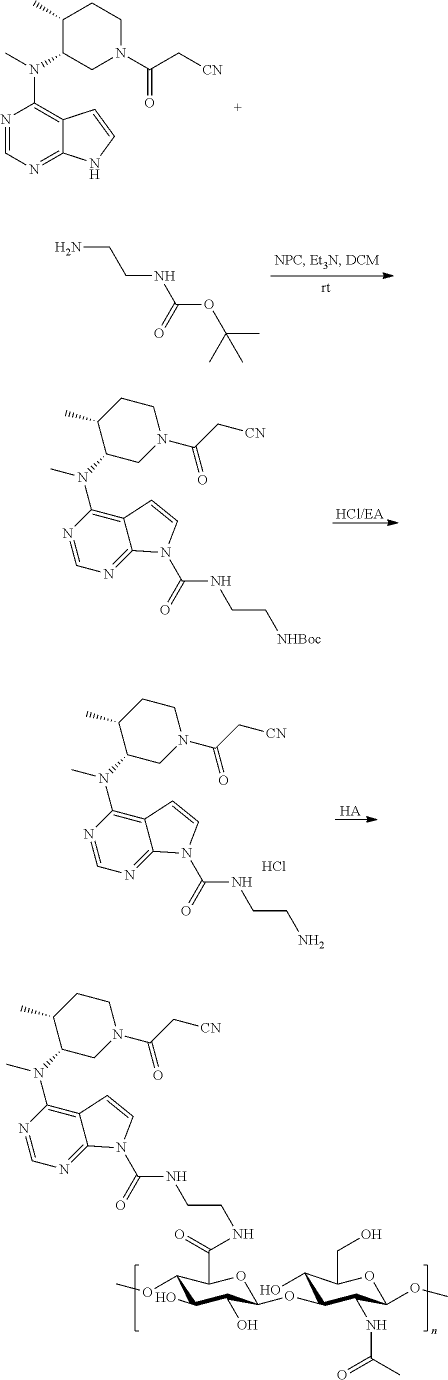

### Step 1: Preparation of tert-butyl (2-(4-(((3R,4R)-1-(2-cyanoacetyl)-4-methylpiperidin-3-yl)(methyl)amino)-7H-pyrrolo[2,3-d]pyrimidine-7-carboxamido) ethyl)carbamate

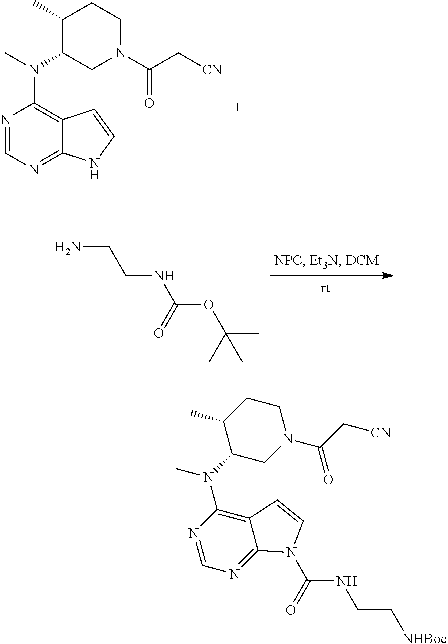

Following Step 1 of Example 1, tofacitinib (3.2 g, 10.24 mmol, 1 eq) and tert-butyl (2-aminoethyl) carbamate (1.64 g, 10.24 mmol, 1 eq) gave the title product (3 g, yield: 59%); MS (m/z): [M+H]+ calcd for C₂₄H₃₄N₈O₄, 499.27; found, 499.1.

### Step 2: Preparation of N-(2-aminoethyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride

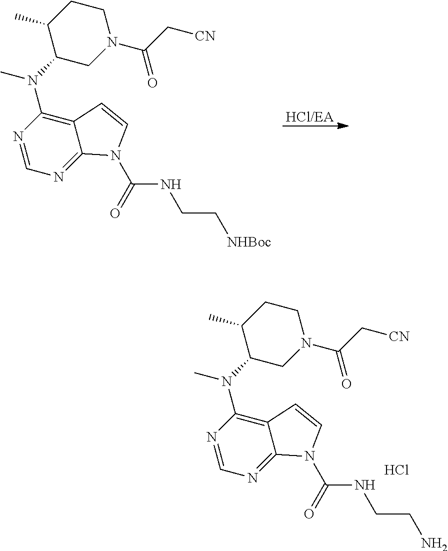 [SMILES: C[C@@H]1CCN(C(=O)CC#N)C[C@@H]1N(C)c1ncnc2c1ccn2C(=O)NCCNC(=O)OC(C)(C)C ; "HCl/EA" ; C[C@@H]1CCN(C(=O)CC#N)C[C@@H]1N(C)c1ncnc2c1ccn2C(=O)NCCN ; Cl]

Following Step 2 in Example 1, tert-butyl (2-(4-(((3R,4R)-1-(2-cyanoacetyl)-4-methylpiperidin-3-yl)(methyl)amino)-7H-pyrrolo[2,3-d]pyrimidine-7-carboxamido)ethyl)carbamate (1 g, 2 mmol, 1 eq) give the desired product as 1-C salt (870 mg, yield: 100%); MS (m/z): [M+H]+ calcd for C₁₉H₂₆N₈O₂, 399.22; found, 399.1. ¹H-NMR (400 MHz, CD₃OD) δ ppm 8.49 (s, 1H), 7.95 (d, J=3.6 Hz, 1H), 7.08 (s, 1H), 4.08-3.73 (m, 7.5H), 3.71-3.41 (m, 4.5H), 3.27 (t, 2H), 2.66-2.44 (m, 1H), 2.00-1.67 (m, 2H), 1.16 (d, J=7.0 Hz, 3H).

### Step 3: Preparation of conjugate of N-(2-aminoethyl)-4-(((3R,4R)-1-(2-cyanoacetyl)-4-methylpiperidin-3-yl)(methyl)amino)-7H-pyrrolo[2,3-d]pyrimidine-7-carboxamide and HA

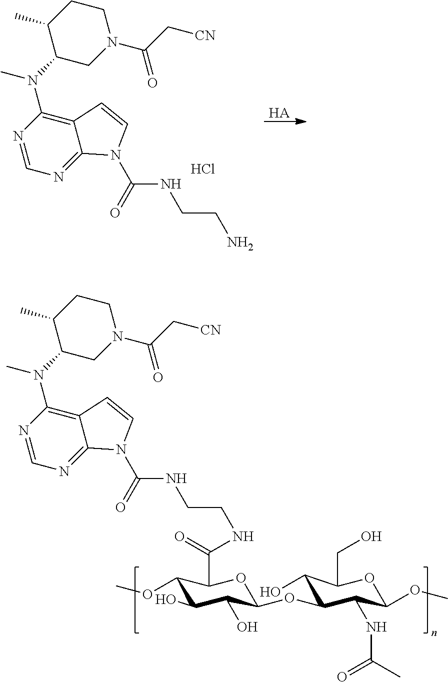

Following Step 3 in Example 1, sodium hyaluronate (MW 50 KDa, 0.186 g, 0.46 mmol, 1 eq) and N-(2-aminoethyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride (0.2 g, 0.46 mmol, 1 eq) afforded the title compound (0.23 g, yield: 66%, DSR=18%). ¹H-NMR (400 MHz, D₂O) δ ppm: 8.23 (m, 0.18H), 7.63 (m, 0.18H), 6.80 (m, 0.18H), 4.64-4.24 (m, 2.2H), 4.05-2.96 (m, 12.3H), 2.42 (m, 0.18H), 1.98 (s, 3H), 1.80-1.67 (m, 0.18H), 1.26 (m, 0.18H), 1.05 (m, 0.54H). ¹H-NMR (400 MHz, D₂O) δ ppm: 8.5-7.9 (m, 0.18H), 7.7-7.0 (m, 0.18H), 7.0-5.8 (m, 0.18H), 4.64-4.24 (m, 2.2H), 4.05-2.96 (m, 12.3H), 2.42 (br, 0.18H), 1.98 (s, 3H), 1.80-1.67 (m, 0.18H), 1.26 (br, 0.18H), 1.05 (m, 0.54H).

### Example 8

### Preparation of conjugate of 4-(((3R,4R)-1-(2-cyanoacetyl)-4-methylpiperidin-3-yl)(methyl)amino)-N-(4-(methylamino)butyl)-7H-pyrrolo[2,3-d]pyrimidine-7-carboxamide and HA

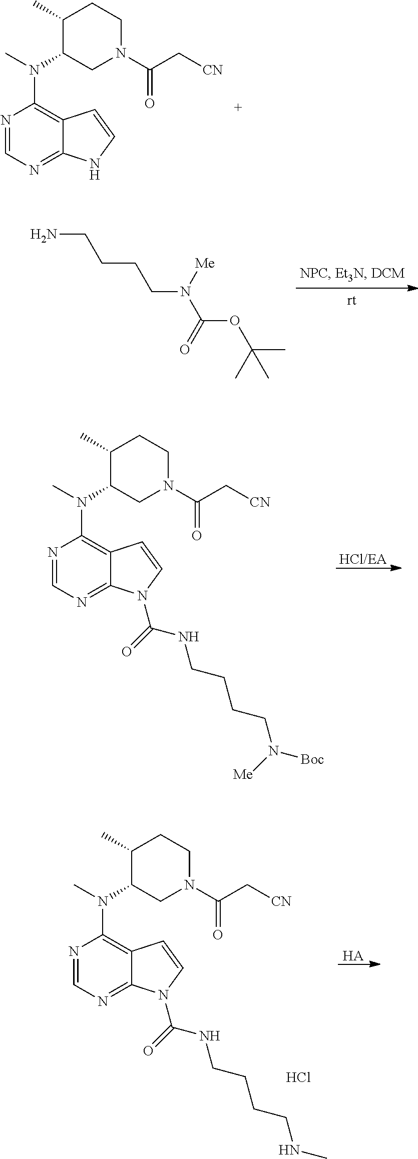

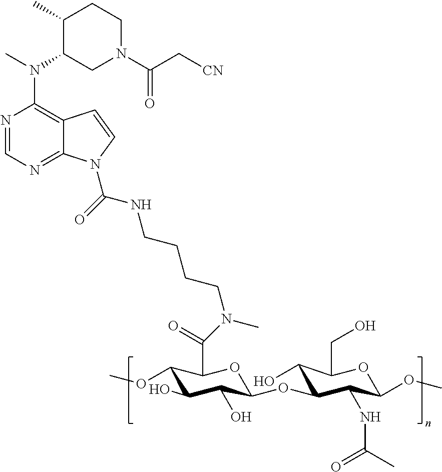

### Step 1: Preparation of tert-butyl (4-(4-(((3R,4R)-1-(2-cyanoacetyl)-4-methylpiperidin-3-yl)(methyl)amino)-7H-pyrrolo[2,3-d]pyrimidine-7-carboxamido)butyl)(methyl)carbamate

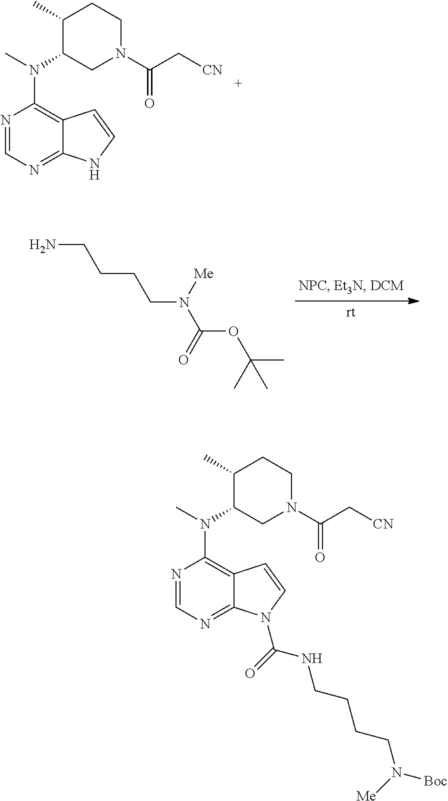 [SMILES: C[C@@H]1CCN(C(=O)CC#N)C[C@@H]1N(C)c1ncnc2[nH]ccc12 ; CN(CCCCN)C(=O)OC(C)(C)C ; C[C@@H]1CCN(C(=O)CC#N)C[C@@H]1N(C)c1ncnc2c1ccn2C(=O)NCCCCN(C)C(=O)OC(C)(C)C]

Following Step 1 of Example 1, tofacitinib (1 g, 3.2 mmol, 1 eq) and tert-butyl (4-aminobutyl)(methyl)carbamate (0.65 g, 3.2 mmol, 1 eq) gave the title product (1 g, yield: 58%); MS (m/z): [M+H]+ calcd for C₂₇H₄N₈O₄, 541.32; found, 541.2.

### Step 2: Preparation of 4-(((3R,4R)-1-(2-cyanoacetyl)-4-methylpiperidin-3-yl)(methyl)amino)-N-(4-(methylamino)butyl)-7H-pyrrolo[2,3-d]pyrimidine-7-carboxamide hydrochloride

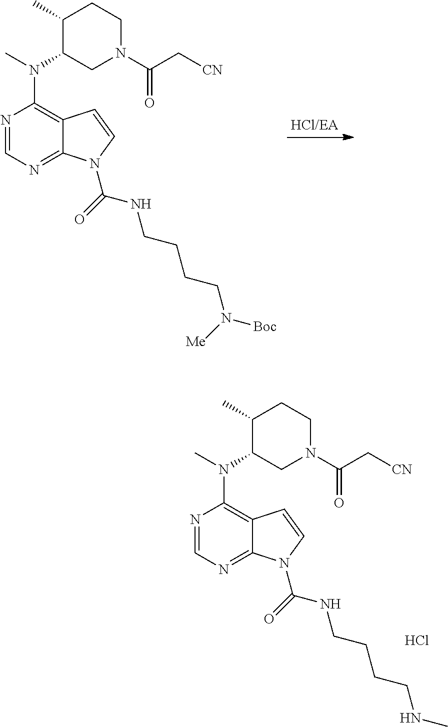 [SMILES: C[C@@H]1CCN(C(=O)CC#N)C[C@@H]1N(C)c1ncnc2c1ccn2C(=O)NCCCCN(C)C(=O)OC(C)(C)C ; CNCCCCNC(=O)n1ccc2c(N(C)[C@H]3CN(C(=O)CC#N)CC[C@H]3C)ncnc21 ; Cl]

Following Step 2 of Example 1, tert-butyl (4-(4-(((3R,4R)-1-(2-cyanoacetyl)-4-methylpiperidin-3-yl)(methyl)amino)-7H-pyrrolo[2,3-d]pyrimidine-7-carboxamido)butyl)(methyl) carbamate (1 g, 1.85 mmol, 1 eq) gave the desired product as HCl salt (0.88 g, yield: 100%); MS (m/z): [M+H]+ calcd for C₂₂H₃₂N₈O₂, 441.26; found, 441.1. ¹H-NMR (400 MHz, CD₃OD) δ ppm 8.50 (d, J=4.7 Hz, 1H), 7.94 (d, J=3.7 Hz, 1H), 7.07 (s, 1H), 4.20-3.81 (m, 3H), 3.73-3.32 (m, 8H), 3.08 (t, 2H), 2.72 (s, 3H), 2.55 (br, 1H), 1.99 (br, 1H), 1.91-1.67 (m, 5H), 1.16 (d, J=6.9, 5.2 Hz, 3H).

### Step 3: Preparation of conjugate of 4-(((3R,4R)-1-(2-cyanoacetyl)-4-methylpiperidin-3-yl)(methyl)amino)-N-(4-(methylamino)butyl)-7H-pyrrolo[2,3-d]pyrimidine-7-carboxamide and HA

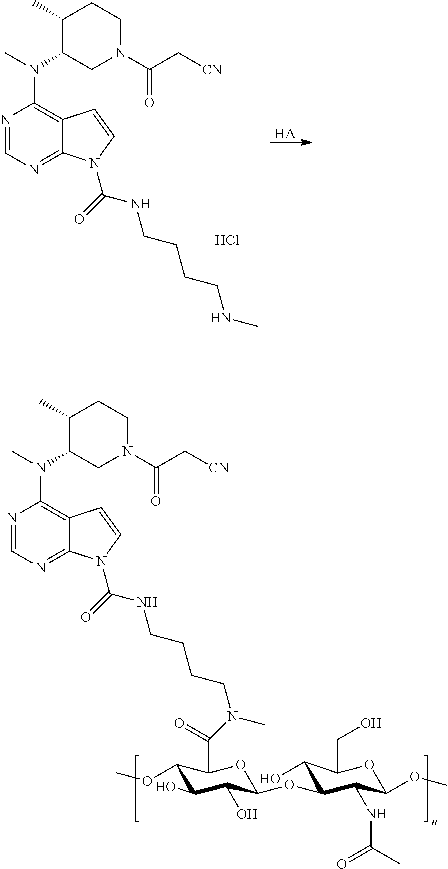

Following Step 3 of Example 1, sodium hyaluronate (MW 50 KDa, 0.169 g, 0.42 mmol, 1 eq) and 4-(((3R,4R)-1-(2-cyanoacetyl)-4-methylpiperidin-3-yl)(methyl)amino)-N-(4-(methylamino)butyl)-7H-pyrrolo[2,3-d]pyrimidine-7-carboxamide hydrochloride (0.2 g, 0.42 mmol, 1 eq) afforded the title compound (0.236 g, yield: 34.4%, DSR=10%); ¹H-NMR (400 MHz, D₂O) δ ppm 8.4-7.9 (m, 0.1H), 7.75-7.35 (m, 0.1H), 6.95-6.5 (m, 0.1H), 4.7-4.2 (m, 2.7H), 4.0-3.2 (m, 11H), 2.47 (br, 0.1H), 2.02 (br, 3H), 1.29 (br, 0.2H), 1.2-0.95 (m, 0.3H).

With Step 3 of Example 1, reaction of sodium hyaluronate (MW 500 KDa) provided corresponding product (0.18 g, yield: 52.3%, DSR=6%).

With Step 3 of Example 1, reaction of sodium hyaluronate (MW 2000 KDa) provided corresponding product KDa (0.17 g, yield: 49.4%, DSR=3%).

### Example 9

### Preparation of conjugate of 4-(((3R,4R)-1-(2-cyanoacetyl)-4-methylpiperidin-3-yl)(methyl)amino)-N′-methyl-7H-pyrrolo[2,3-d]pyrimidine-7-carbohydrazide and HA

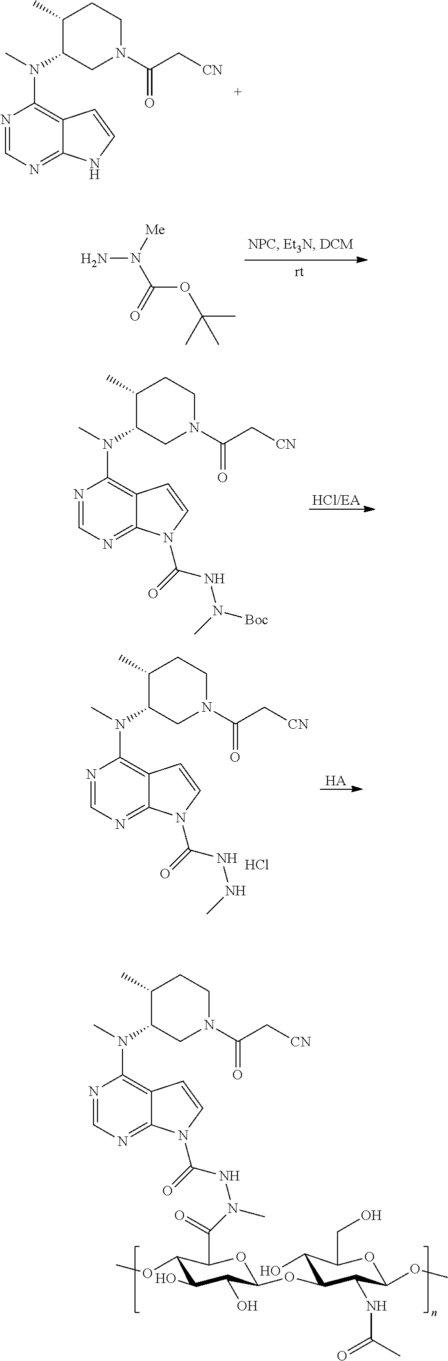

### Step 1: Preparation of tert-butyl 2-(4-(((3R,4R)-1-(2-cyanoacetyl)-4-methylpiperidin-3-yl)(methyl)amino)-7H-pyrrolo[2,3-d]pyrimidine-7-carbonyl)-1-methylhydrazine-1-carboxylate

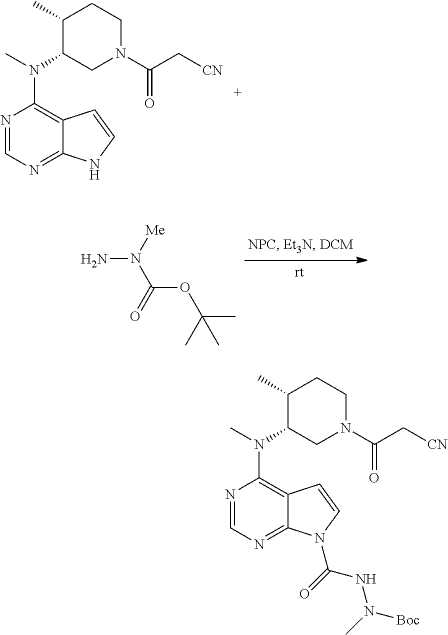

Following Step 1 of Example 1, tofacitinib (3.124 g, 10 mmol, 1 eq) and tert-butyl 1-methylhydrazine-1-carboxylate (1.45 g, 10 mmol, 1 eq) gave the title product (2.2 g, yield: 46%); MS (m/z): [M+H]+ calcd for C₂₃H₃₂N₈O₄, 485.25; found, 485.2.

### Step 2: Preparation of 4-(((3R,4R)-1-(2-cyanoacetyl)-4-methylpiperidin-3-yl)(methyl)amino)-N′-methyl-7H-pyrrolo[2,3-d]pyrimidine-7-carbohydrazide hydrochloride

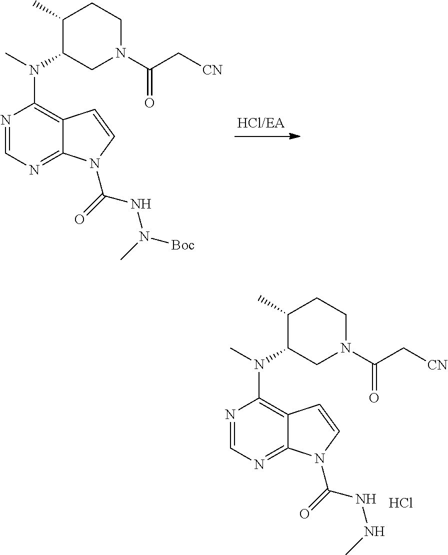 [SMILES: C[C@@H]1CCN(C(=O)CC#N)C[C@@H]1N(C)c1ncnc2c1ccn2C(=O)NN(C)C(=O)OC(C)(C)C ; CNNC(=O)n1ccc2c(N(C)[C@H]3CN(C(=O)CC#N)CC[C@H]3C)ncnc21 ; Cl]

Following Step 2 of Example 1, tert-butyl 2-(4-(((3R,4R)-1-(2-cyanoacetyl)-4-methylpiperidin-3-yl)(methyl)amino)-7H-pyrrolo[2,3-d]pyrimidine-7-carbonyl)-1-methylhydrazine-1-carboxylate (1 g, 2.06 mmol, 1 eq) gave the desired product as HCl salt (870 mg, yield: 100%); MS (m/z): [M+H]+ calcd for C₁₈H₂₄N₈O₂, 385.20; found, 385.1. ¹H-NMR (400 MHz, D₂O) δ ppm 8.44 (d, J=5.0 Hz, 1H), 7.79 (d, J=3.5 Hz, 1H), 6.98 (s, 1H), 4.75 (s, 1H), 4.15-3.79 (m, 4H), 3.72-3.32 (m, 5H), 3.03 (s, 3H), 2.55 (br, 1H), 1.97 (br, 1H), 1.83 (br, 1H), 1.13 (dd, J=15.1, 7.1 Hz, 3H).

### Step 3: Preparation of conjugate of 4-(((3R,4R)-1-(2-cyanoacetyl)-4-methylpiperidin-3-yl)(methyl)amino)-N′-methyl-7H-pyrrolo[2,3-d]pyrimidine-7-carbohydrazide and HA

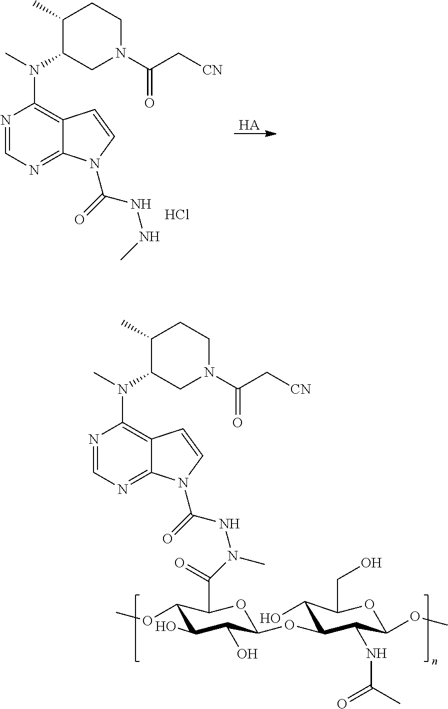

Following Step 2 of Example 1, sodium hyaluronate (MW 50 KDa, 0.192 g, 0.475 mmol, 1 eq) and 4-(((3R,4R)-1-(2-cyanoacetyl)-4-methylpiperidin-3-yl)(methyl)amino)-N′-methyl-7H-pyrrolo[2,3-d]pyrimidine-7-carbohydrazide hydrochloride (0.2 g, 0.475 mmol, 1 eq) afforded the title compound (0.323 g, yield: 90%, DSR=15%); ¹H-NMR (400 MHz, D₂O) δ ppm 8.4-8.2 (m, 0.15H), 7.8-7.5 (m, 0.15H), 7.05-6.75 (m, 0.15H), 4.75-4.2 (m, 2.9H), 4.0-3.2 (m, 11H), 2.47 (br, 0.15H), 2.02 (br, 3H), 1.30 (br, 0.15H), 1.08 (m, 0.45H).

With Step 3 of Example 1, reaction of sodium hyaluronate (MW 500 KDa) provided corresponding product (0.303 g, yield: 83%, DSR=10%).

With Step 3 of Example 1, reaction of sodium hyaluronate (MW 2000 KDa) provided corresponding product (0.327 g, yield: 90.3%, DSR=15%).

### Example 10

### Preparation of conjugate of N′-allyl-4-(((3R,4R)-1-(2-cyanoacetyl)-4-methylpiperidin-3-yl)(methyl)amino)-7H-pyrrolo[2,3-d]pyrimidine-7-carbo hydrazide and HA

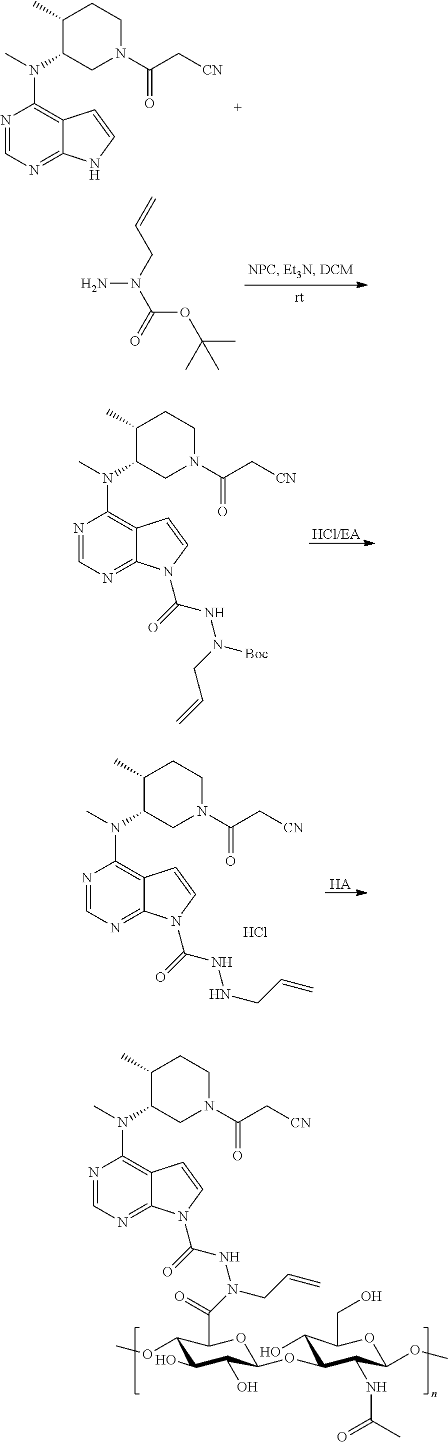

### Step 1: Preparation of tert-butyl 1-allyl-2-(4-(((3R,4R)-1-(2-cyanoacetyl)-4-methyl piperidin-3-yl)(methyl)amino)-7H-pyrrolo[2,3-d]pyrimidine-7-carbonyl) hydrazine-1-carboxylate

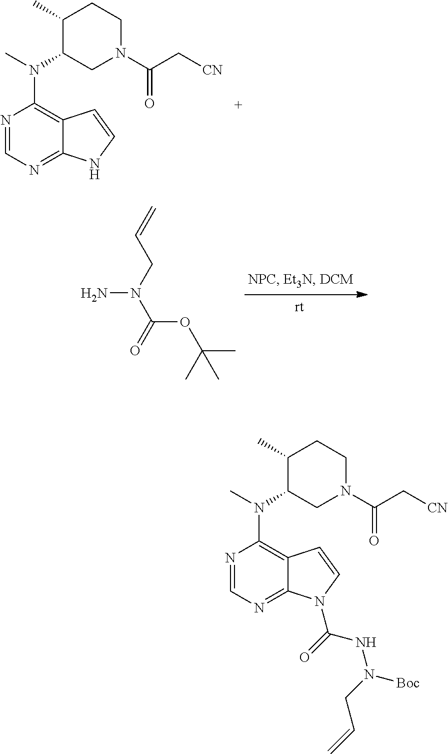 [SMILES: C[C@@H]1CCN(C(=O)CC#N)C[C@@H]1N(C)c1ncnc2[nH]ccc12 ; C=CCN(N)C(=O)OC(C)(C)C ; C=CCN(NC(=O)n1ccc2c(N(C)[C@H]3CN(C(=O)CC#N)CC[C@H]3C)ncnc21)C(=O)OC(C)(C)C]

Following Step 1 of Example 1, tofacitinib (2 g, 6.4 mmol, 1 eq) tert-butyl 1-methylhydrazine-1-carboxylate (1.1 g, 6.4 mmol, 1 eq) gave the title product (0.8 g, yield: 25%); MS (m/z): [M+H]+ calcd for C25H34N8O4, 511.27; found, 511.1.

### Step 2: Preparation of N′-allyl-4-(((3R,4R)-1-(2-cyanoacetyl)-4-methylpiperidin-3-yl)(methyl)amino)-7H-pyrrolo[2,3-d]pyrimidine-7-carbohydrazide Hydrochloride

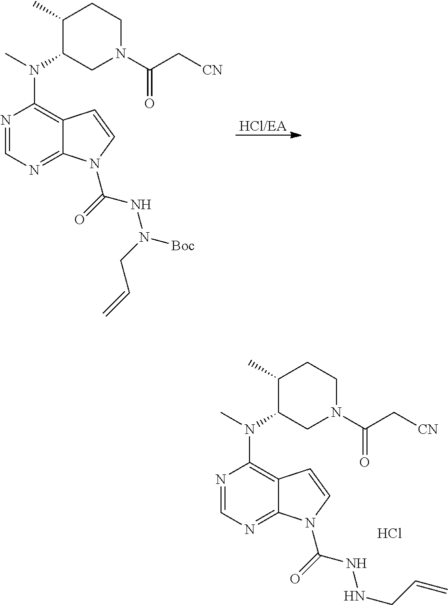 [SMILES: C=CCN(NC(=O)n1ccc2c(N(C)[C@H]3CN(C(=O)CC#N)CC[C@H]3C)ncnc21)C(=O)OC(C)(C)C ; "HCl/EA" ; C=CCNNC(=O)n1ccc2c(N(C)[C@H]3CN(C(=O)CC#N)CC[C@H]3C)ncnc21 ; Cl]

Following Step 2 of Example 1, tert-butyl 1-allyl-2-(4-(((3R,4R)-1-(2-cyanoacetyl)-4-methylpiperidin-3-yl)(methyl)amino)-7H-pyrrolo[2,3-d]pyrimidine-7-carbonyl)hydrazine-1-carboxylate (0.8 g, 1.57 mmol) gave the desired product as HCl salt (0.67 g, yield: 96%); MS (m/z): [M+H]+ calcd for C₂₀H₂₆N₈O₂, 411.22; found, 411.1. ¹H-NMR (400 MHz, CD₃OD) δ ppm 8.50 (d, J=4.2 Hz, 1H), 7.86 (s, 1H), 7.10 (d, J=3.8 Hz, 1H), 6.08-5.98 (m, 1H), 5.61-5.52 (m, 2H), 4.34-3.73 (m, 6H), 3.73-3.32 (m, 6H), 2.53 (br, 1H), 2.05-1.9 (m, 1H), 1.87-1.64 (m, 1H), 1.17 (d, J=7.0 Hz 3H).

### Step 3: Preparation of conjugate of N′-allyl-4-(((3R,4R)-1-(2-cyanoacetyl)-4-methylpiperidin-3-yl)(methyl)amino)-7H-pyrrolo[2,3-d]pyrimidine-7-carbohydrazide and HA

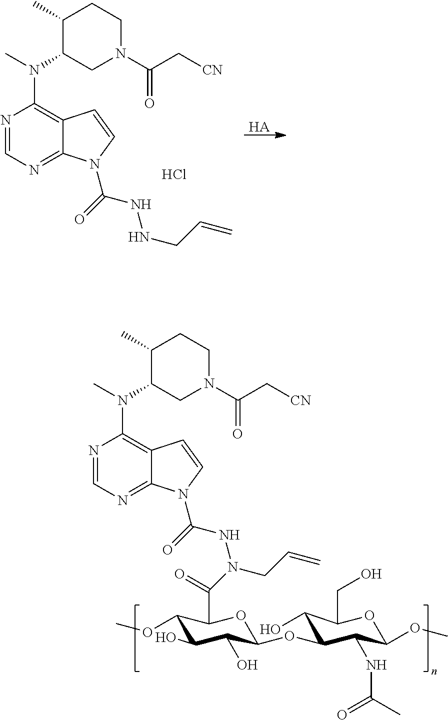

Following Step 3 of Example 1, sodium hyaluronate (MW 50 KDa, 0.182 g, 0.45 mmol, 1 eq) and N′-allyl-4-(((3R,4R)-1-(2-cyanoacetyl)-4-methyl piperidin-3-yl)(methyl)amino)-7H-pyrrolo[2,3-d]pyrimidine-7-carbo hydrazide hydrochloride (0.2 g, 0.45 mmol, 1 eq) afforded the title compound (0.172 g, yield: 48.6%, DSR=5%); ¹H-NMR (400 MHz, D₂O) δ ppm 8.50-8.17 (m, 0.05H), 7.85-7.55 (m, 0.05H), 7.05-6.8 (m, 0.05H), 6.17-5.60 (m, 0.15H), 4.66-4.31 (m, 2H), 4.08-3.29 (m, 10.6H), 2.47 (br, 0.05H), 2.01 (s, 3H), 1.30 (m, 0.1H), 1.19-0.98 (m, 0.15H).

With Step 3 of Example 1, reaction of sodium hyaluronate (MW 500 KDa) provided corresponding product (0.175 g, yield: 49%, DSR=3%)

With Step 3 of Example 1, reaction of sodium hyaluronate (MW 2000 KDa) provided corresponding product (0.186 g, yield: 52.3%, DSR=1%).

### Example 11

### Preparation of conjugate of N-(5-(aminomethyl)pyridin-2-yl)-4-(((3R,4R)-1-(2-cyanoacetyl)-4-methylpiperidin-3-yl)(methyl)amino)-7H-pyrrolo[2,3-d]pyrimidine-7-carboxamide and HA

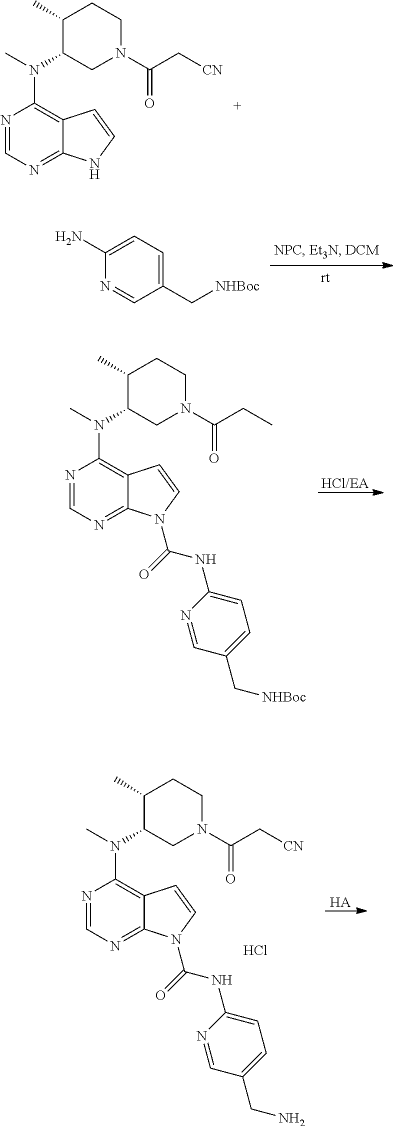

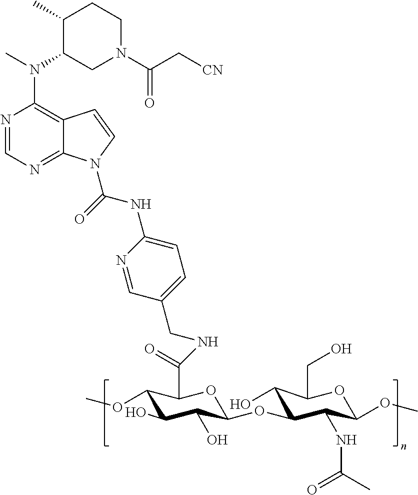

### Step 1: Preparation of tert-butyl ((6-(4-(((3R,4R)-1-(2-cyanoacetyl)-4-methylpiperidin-3-yl) (methyl)amino)-7H-pyrrolo[2,3-d]pyrimidine-7-carboxamido)pyridin-3-yl)methyl)carbamate

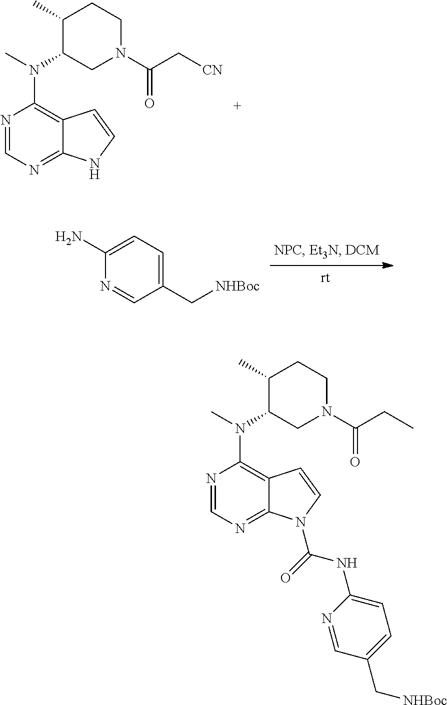 [SMILES: C[C@@H]1CCN(C(=O)CC#N)C[C@@H]1N(C)c1ncnc2[nH]ccc12 ; CC(C)(C)OC(=O)NCc1ccc(N)nc1 ; CCC(=O)N1CC[C@@H](C)[C@@H](N(C)c2ncnc3c2ccn3C(=O)Nc2ccc(CNC(=O)OC(C)(C)C)cn2)C1]

Following Step 1 of Example 1, tofacitinib (0.9 g, 2.88 mmol, 1 eq) and tert-butyl ((6-aminopyridin-3-yl)methyl)carbamate (0.643 g, 2.88 mmol, 1 eq) gave the title product (1 g, yield: 62.50%); MS (m/z): [M+H]+ calcd for C₂₈H₃₅N₉O₄, 526.28; found, 526.1.

### Step 2: Preparation of N-(5-(aminomethyl)pyridin-2-yl)-4-(((3R,4R)-1-(2-cyanoacetyl)-4-methylpiperidin-3-yl)(methyl)amino)-7H-pyrrolo[2,3-d]pyrimidine-7-carboxamide hydrochloride

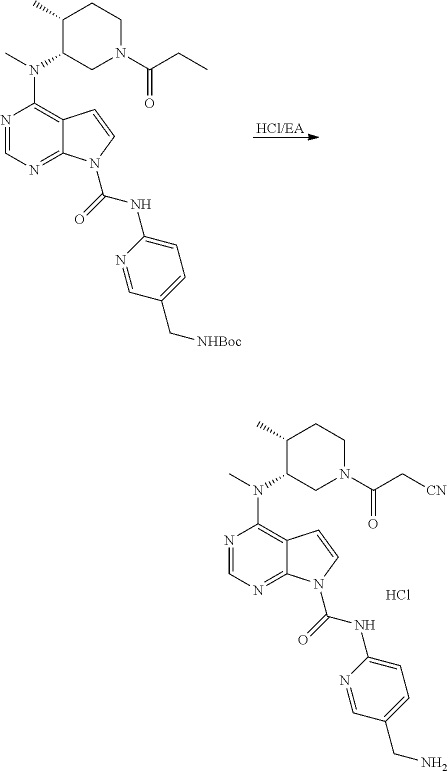 [SMILES: CCC(=O)N1CC[C@@H](C)[C@@H](N(C)c2ncnc3c2ccn3C(=O)Nc2ccc(CNC(=O)OC(C)(C)C)cn2)C1 ; "HCl/EA" ; C[C@@H]1CCN(C(=O)CC#N)C[C@@H]1N(C)c1ncnc2c1ccn2C(=O)Nc1ccc(CN)cn1 ; Cl]

Following Step 2 of Example 1, tert-butyl((6-(4-(((3R,4R)-1-(2-cyanoacetyl)-4-methylpiperidin-3-yl)(methyl)amino)-7H-pyrrolo[2,3-d]pyrimidine-7-carboxamido)pyridin-3-yl)methyl) carbamate (0.8 g, 1.42 mmol, 1 eq) gave the desired product as HCl salt (0.7 g, yield: 100%); MS (m/z): [M+H]+ calcd for C₂₃H₂₇N₉O₂, 462.23; found, 462.1. ¹H-NMR (400 MHz, D₂O) δ ppm 8.41-8.31 (m, 2H), 8.1-7.9 (m, 3H), 7.78-7.68 (m, 1H), 6.83 (d, J=2.4 Hz, 2H), 4.67-4.49 (m, 1H), 4.26 (s, 2H), 4.14-3.82 (m, 5H), 3.66-3.51 (m, 2H), 3.45-3.2 (m, 3H), 2.49 (br, 1H), 2.03-1.85 (m, 1H), 1.77 (m, 1H), 1.13 (dd, J=24.3, 6.3 Hz, 3H).

### Step 3: Preparation of conjugate of N-(5-(aminomethyl)pyridin-2-yl)-4-(((3R,4R)-1-(2-cyanoacetyl)-4-methylpiperidin-3-yl)(methyl)amino)-7H-pyrrolo[2,3-d] pyrimidine-7-carboxamide and HA

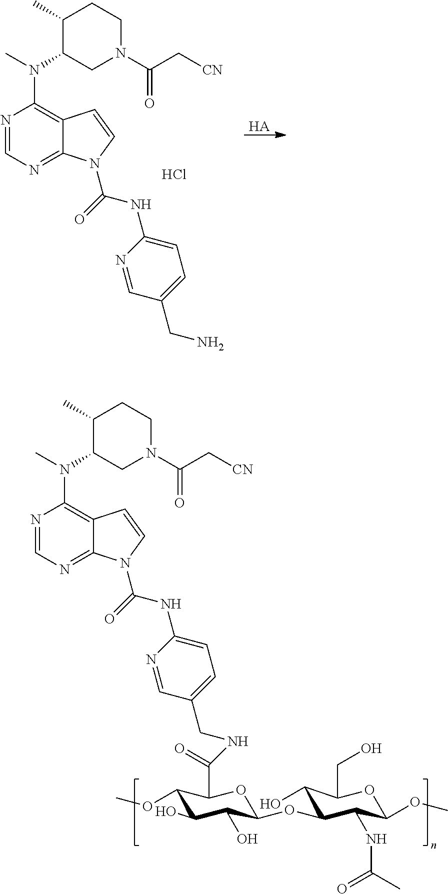

Following Step 3 of Example 1, sodium hyaluronate (MW 50 KDa, 0.161 g, 0.4 mmol, 1 eq) and N-(5-(aminomethyl)pyridin-2-yl)-4-(((3R,4R)-1-(2-cyanoacetyl)-4-methylpiperidin-3-yl)(methyl)amino)-7H-pyrrolo[2,3-d]pyrimidine-7-carboxamide hydro chloride (0.2 g, 0.4 mmol, 1 eq) afforded the title compound (0.17 g, yield: 50%, DSR=20.5%); ¹H-NMR (400 MHz, D₂O) δ ppm 8.6-7.3 (m, 1.12H), 7.2-6.8 (m, 0.11H), 4.7-4.3 (m, 2H), 4.02-3.17 (m, 10.46H), 2.40 (br, 0.21H), 2.00 (s, 3H), 1.61 (br, 0.21H), 1.35-1.2 (m, 0.21H), 1.15-0.85 (m, 0.65H).

With Step 3 of Example 1, reaction of sodium hyaluronate (MW 2000 KDa) provided corresponding product (0.177 g, yield: 52.6%, DSR=22.2%).

### Example 12

### Preparation of conjugate of 4-(((3R, 4R)-1-(2-cyanoacetyl)-4-methylpiperidin-3-yl)(methyl)amino)-N-(4-(piperazin-1-yl)phenyl)-7H-pyrrolo[2,3-d]pyrimidine-7-carboxamide and HA

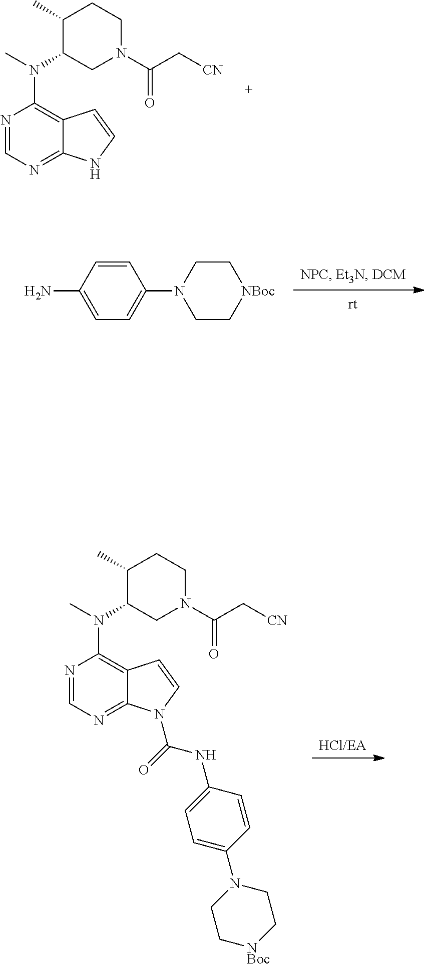

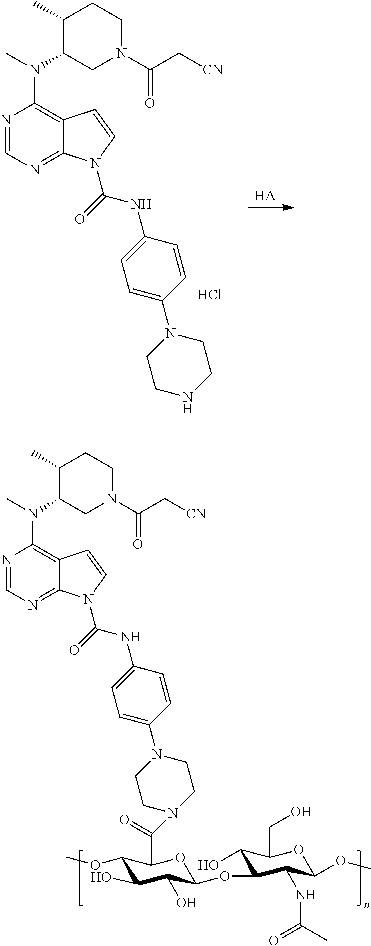

### Step 1: Preparation of tert-butyl 4-(4-(4-(((3R,4R)-1-(2-cyanoacetyl)-4-methylpiperidin-3-yl)(methyl)amino)-7H-pyrrolo[2,3-d]pyrimidine-7-carboxamido)phenyl)piperazine-1-carboxylate

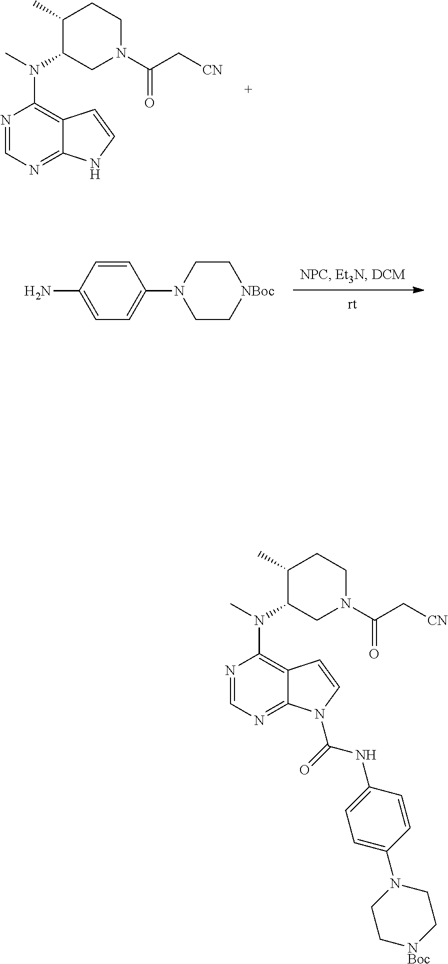

Following Step 1 of Example 1, tofacitinib (0.52 g, 1.66 mmol, 1 eq) tert-butyl 4-(4-aminophenyl)piperazine-1-carboxylate (0.46 g, 1.66 mmol, 1 eq) gave the title product (0.65 g, yield: 65%); MS (m/z): [M+H]+ calcd for C₃₂H₄₁N₉O₄, 616.33; found, 616.1.

### Step 2: Preparation of 4-(((3R,4R)-1-(2-cyanoacetyl)-4-methylpiperidin-3-yl)(methyl)amino)-N-(4-(piperazin-1-yl)phenyl)-7H-pyrrolo[2,3-d]pyrimidine-7-carboxamide hydrochloride

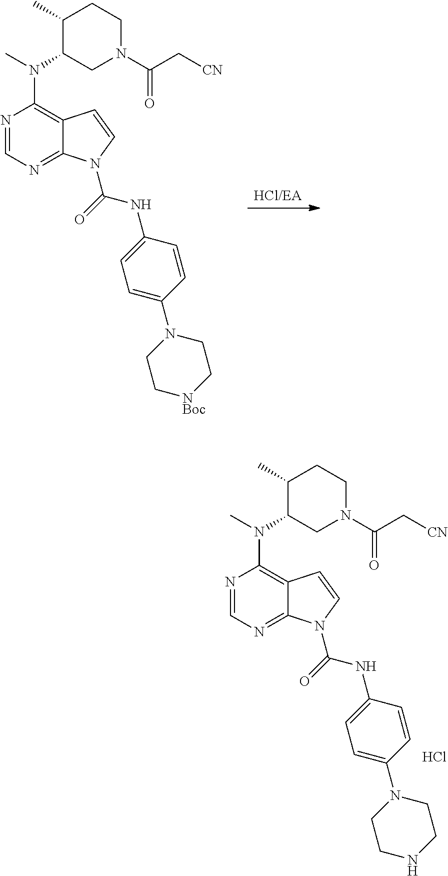 [SMILES: C[C@@H]1CCN(C(=O)CC#N)C[C@@H]1N(C)c1ncnc2c1ccn2C(=O)Nc1ccc(N2CCN(C(=O)OC(C)(C)C)CC2)cc1 ; "HCl/EA" ; C[C@@H]1CCN(C(=O)CC#N)C[C@@H]1N(C)c1ncnc2c1ccn2C(=O)Nc1ccc(N2CCNCC2)cc1 ; Cl]

Following Step 2 of Example 1, tert-butyl 4-(4-(4-(((3R,4R)-1-(2-cyanoacetyl)-4-methyl piperidin-3-yl)(methyl)amino)-7H-pyrrolo[2,3-d]pyrimidine-7-carboxamido)phenenyl)piperazine-1-carboxylate (0.6 g, 0.975 mmol, 1 eq) gave the desired product as HCl salt (0.538 mg, yield: 100%); MS (m/z): [M+H]+ calcd for C₂₇H₃₃N₉O₂, 516.28; found, 516.1. ¹H-NMR (400 MHz, D₂O) δ ppm 8.28 (s, 1H), 7.68 (s, 1H), 7.55-7.4 (m, 2H), 7.25-7.10 (m, 2H), 6.78 (s, 1H), 4.55 (s, 1H), 4.12-2.92 (m, 17H), 2.47 (br, 1H), 2.0-1.85 (m, 1H), 1.85-1.65 (m, 1H), 1.11 (dd, J=16.6, 7.0 Hz, 3H).

### Step 3: Preparation of conjugate of 4-(((3R, 4R)-1-(2-cyanoacetyl)-4-methylpiperidin-3-yl)(methyl)amino)-N-(4-(piperazin-1-yl)phenyl)-7H-pyrrolo[2,3-d]pyrimidine-7-carboxamide and HA

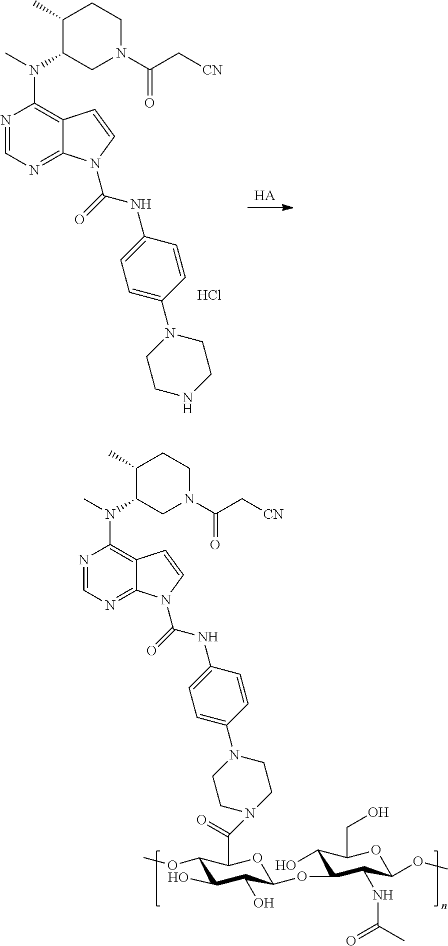

Following Step 3 of Example 1, sodium hyaluronate (MW 50 KDa, 0.145 g, 0.36 mmol, 1 eq) and 4-(((3R,4R)-1-(2-cyanoacetyl)-4-methylpiperidin-3-yl)(methyl)amino-N-(4-(piperazin-1-yl)phenyl)-7H-pyrrolo[2,3-d]pyrimidine-7-carboxamide hydro chloride (0.2 g, 0.36 mmol, leg) afforded the title compound (0.13 g, yield: 41%, DSR=19%); ¹H-NMR (400 MHz, D₂O) δ ppm 8.4-6.8 (m, 1.33H), 4.7-4.4 (m, 2H), 3.9-3.42 (m, 12H), 3.34 (m, 1.42H), 2.47 (br, 0.19H), 2.02 (s, 3H), 1.29 (br, 0.38H), 1.06 (br, 0.57H).

With Step 3 of Example 1, reaction of sodium hyaluronate (MW 2000 KDa) provided corresponding product (0.16 g, yield: 49.7%, DSR=20%).

### Example 13

### Preparation of conjugate of 4-(((3R,4R)-1-(2-cyanoacetyl)-4-methylpiperidin-3-yl)(methyl)amino)-N-(5-(piperazin-1-yl)pyridin-2-yl)-7H-pyrrolo[2,3-d]pyrimidine-7-carboxamide and HA

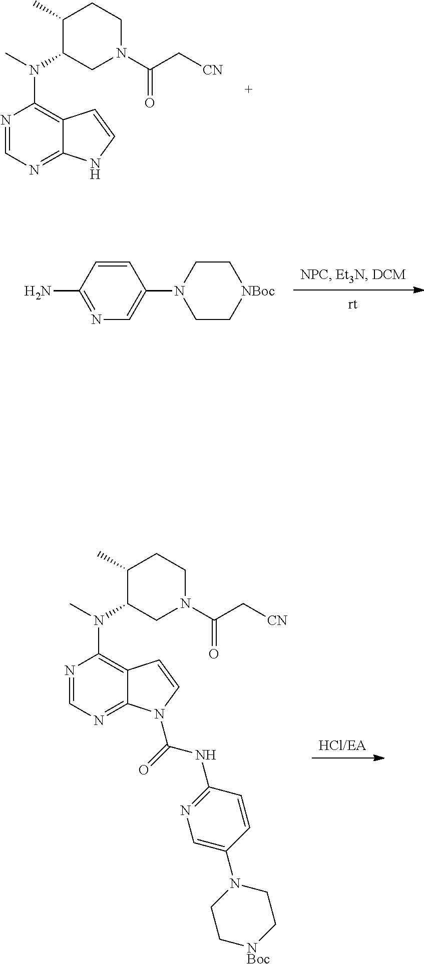

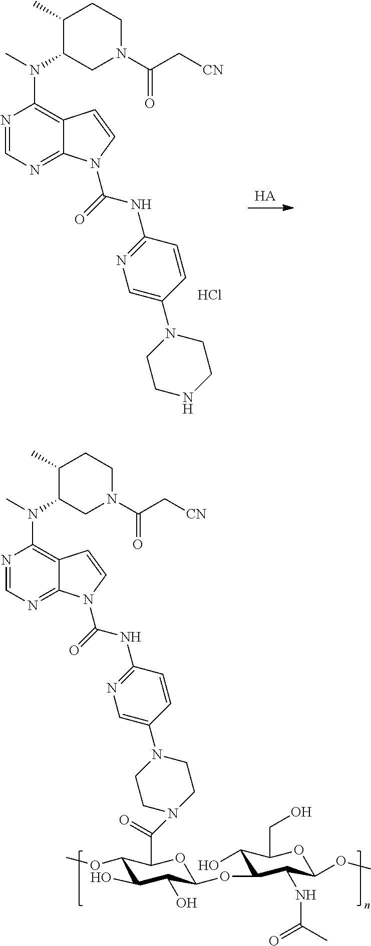

### Step 1: Preparation of tert-butyl 4-(6-(4-(((3R,4R)-1-(2-cyanoacetyl)-4-methylpiperidin-3-yl)(methyl)amino)-7H-pyrrolo[2,3-d]pyrimidine-7-carboxamido)pyridin-3-yl)piperazine-1-carboxylate

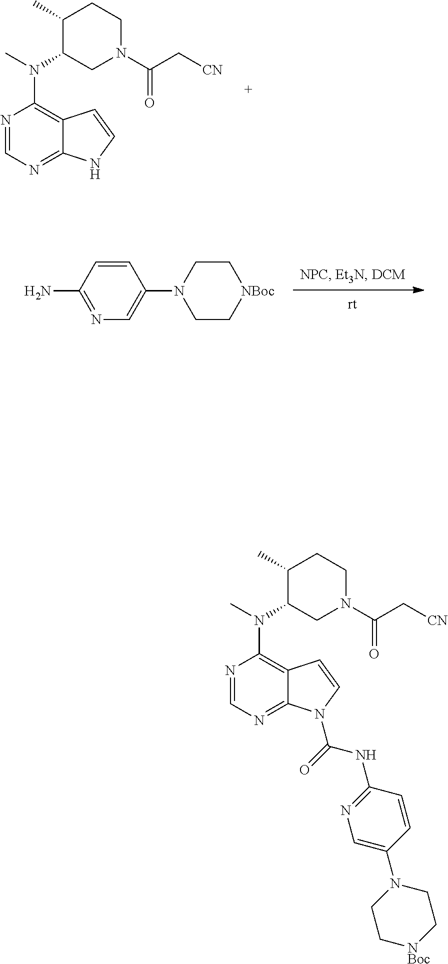

Following Step 1 of Example 1, tofacitinib (1.12 g, 3.59 mmol, 1 eq) and tert-butyl 4-(6-aminopyridin-3-yl) piperazine-1-carboxylate (1 g, 3.59 mmol, 1 eq) gave the title product (1.3 g, yield: 58.7%); MS (m/z): [M+H]+ calcd for C₃₁H₄₀N₁₀O₄, 617.32; found, 617.2.

### Step 2: Preparation of 4-(((3R,4R)-1-(2-cyanoacetyl)-4-methylpiperidin-3-yl)(methyl)amino)-N-(5-(piperazin-1-yl)pyridin-2-yl)-7H-pyrrolo[2,3-d]pyrimidine-7-carboxamide hydrochloride

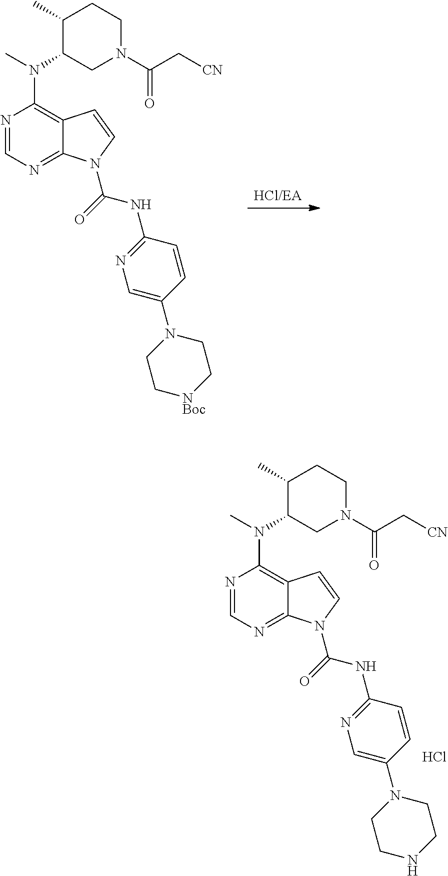 [SMILES: C[C@@H]1CCN(C(=O)CC#N)C[C@@H]1N(C)c1ncnc2c1ccn2C(=O)Nc1ccc(N2CCN(C(=O)OC(C)(C)C)CC2)cn1 ; "HCl/EA" ; C[C@@H]1CCN(C(=O)CC#N)C[C@@H]1N(C)c1ncnc2c1ccn2C(=O)Nc1ccc(N2CCNCC2)cn1 ; Cl]

Following Step 2 of Example 1, tert-butyl 4-(6-(4-(((3R,4R)-1-(2-cyanoacetyl)-4-methyl piperidin-3-yl)(methyl)amino)-7H-pyrrolo[2,3-d]pyrimidine-7-carboxamido)pyridin-3-yl) piperazine-1-carboxylate (1 g, 1.621 mmol, 1 eq) gave the desired product as HCl salt (0.89 g, yield: 99%); MS (m/z): [M+H]+ calcd for C₂₆H₃₂N₁₀O₂, 517.28; found, 517.1. ¹H-NMR (400 MHz, D₂O) δ ppm 8.33 (d, J=11.6 Hz, 1H), 8.04 (d, J=13.2 Hz, 1H), 7.85 (d, J=19.7 Hz, 2H), 7.71 (d, J=8.7 Hz, 1H), 6.83 (s, 1H), 4.62-4.57 (m, 1H), 4.10-3.76 (m, 2H), 3.65-3.21 (m, 15H), 2.47 (m, 1H), 1.94 (m, 1H), 1.77 (m, 1H), 1.12 (dd, J=24.1, 7.0 Hz, 3H).

### Step 3: Preparation of conjugate of 4-(((3R,4R)-1-(2-cyanoacetyl)-4-methylpiperidin-3-yl)(methyl)amino)-N-(5-(piperazin-1-yl)pyridin-2-yl)-7H-pyrrolo[2,3-d]pyrimidine-7-carboxamide and HA

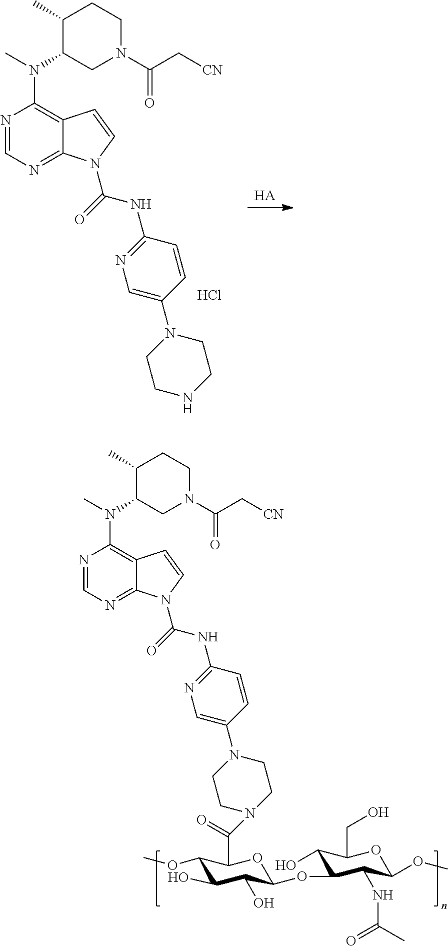

Following Step 3 of Example 1, sodium hyaluronate (MW 50 KDa, 0.145 g, 0.36 mmol, 1 eq) and 4-(((3R,4R)-1-(2-cyanoacetyl)-4-methylpiperidin-3-yl)(methyl)amino)-N-(5-(piperazin-1-yl)pyridin-2-yl)-7H-pyrrolo[2,3-d]pyrimidine-7-carboxamide hydrochloride (0.2 g, 0.36 mmol, 1 eq) afforded the title compound (0.17 g, yield: 33%, DSR=28%); ¹H-NMR (400 MHz, D₂O) δ ppm 8.72-6.98 (m, 1.7H), 4.70-4.3 (m, 2H), 3.80-3.28 (m, 15H), 2.84-2.67 (m, 0.29H), 1.95 (s, 3H), 1.40-1.10 (m, 0.66H), 1.1-0.9 (m, 0.85H).

With Step 3 of Example 1, reaction of sodium hyaluronate (MW 2000 KDa) provided corresponding product (0.22 g, yield: 68%, DSR=20%).

### Example 14

### Preparation of conjugate of N-(4-(2-aminoethoxy)phenyl)-4-(((3R,4R)-1-(2-cyanoacetyl)-4-methylpiperidin-3-yl)(methyl)amino)-7H-pyrrolo[2,3-d]pyrimidine-7-carboxamide and HA

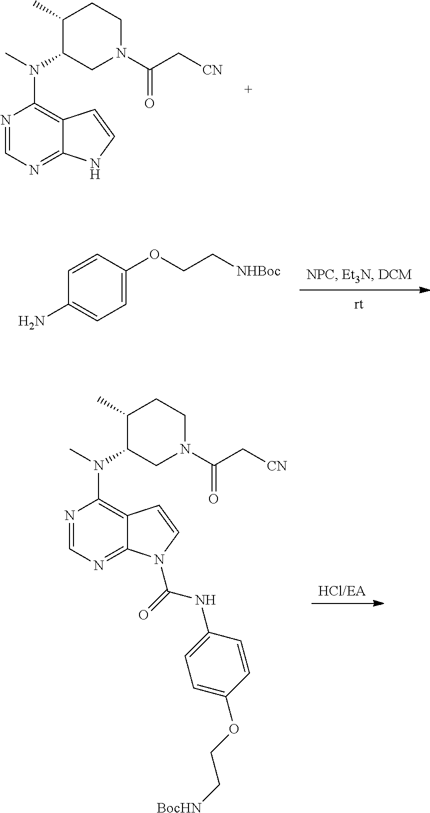

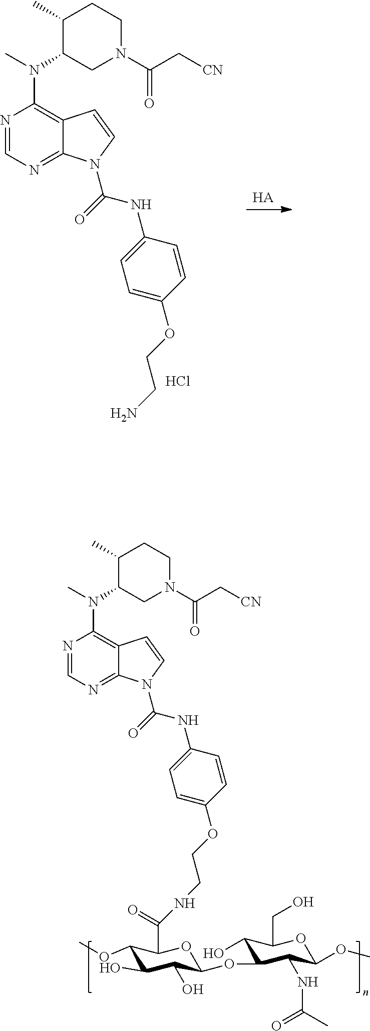

### Step 1: Preparation of tert-butyl (2-(4-(4-(((3R,4R)-1-(2-cyanoacetyl)-4-methylpiperidin-3-yl)(methyl)amino)-7H-pyrrolo[2,3-d]pyrimidine-7-carboxamido) phenoxy)ethyl)carbamate

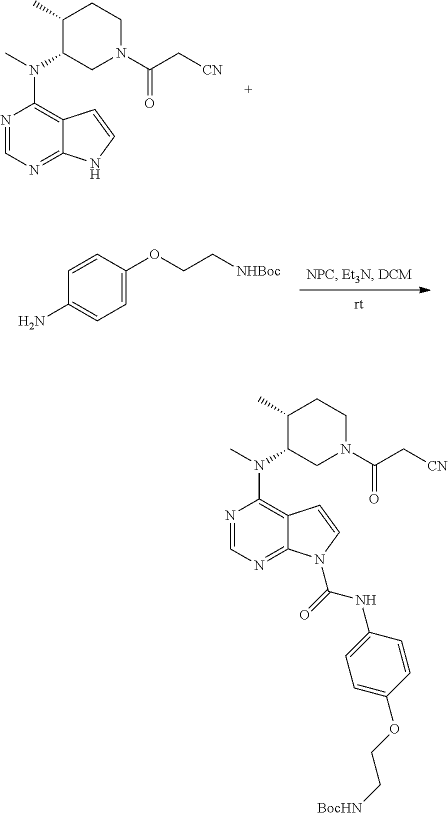

Following Step 1 of Example 1, tofacitinib (1.24 g, 3.96 mmol, 1 eq) and tert-butyl (2-(4-aminophenoxy)ethyl)carbamate (1 g, 3.96 mmol, 1 eq) gave the title product (1.3 g, yield: 55.6%); MS (m/z): [M+H]+ calcd for C₃₀H₃₈NO₅, 591.30; found, 591.2.

### Step 2: Preparation of N-(4-(2-aminoethoxy)phenyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride

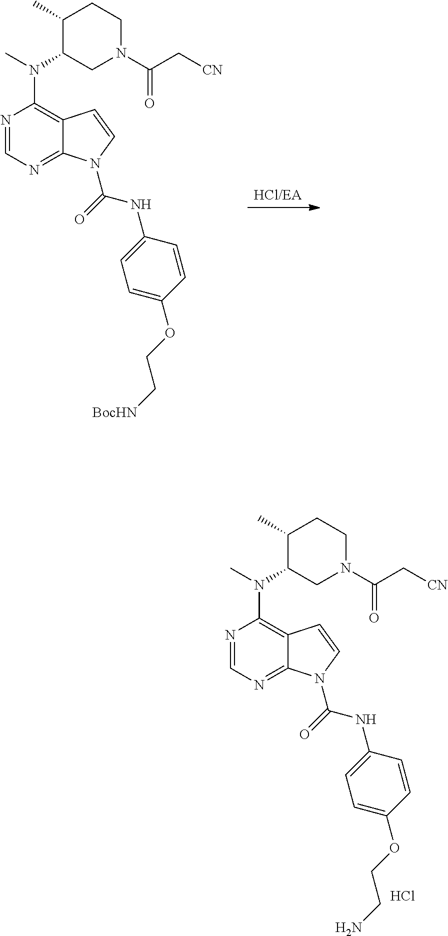 [SMILES: C[C@@H]1CCN(C(=O)CC#N)C[C@@H]1N(C)c1ncnc2c1ccn2C(=O)Nc1ccc(OCCNC(=O)OC(C)(C)C)cc1 ; "HCl/EA" ; C[C@@H]1CCN(C(=O)CC#N)C[C@@H]1N(C)c1ncnc2c1ccn2C(=O)Nc1ccc(OCCN)cc1 ; Cl]

Following Step 2 of Example 1, tert-butyl (2-(4-(4-(((3R,4R)-1-(2-cyanoacetyl)-4-methylpiperidin-3-yl)(methyl)amino)-7H-pyrrolo[2,3-d]pyrimidine-7-carboxamido)phenoxy)ethyl)carbamate (1 g, 1.69 mmol, 1 eq) gave the desired product as HCl salt (0.892 g, yield: 100%); MS (m/z): [M+H-]+ calcd for C₂₅H₃₀N₈O₃, 491.24; found, 491.1. ¹H-NMR (400 MHz, D₂O) δ ppm 8.15 (s, 1H), 7.51 (s, 1H), 7.34 (dd, J=16.5, 8.8 Hz, 2H), 7.03 (t, 2H), 6.63 (s, 1H), 4.45 (s, 1H), 4.38-3.38 (m, 10H), 3.21 (d, J=15.1 Hz, 2H), 2.39 (s, 1H), 2.0-1.6 (m, 2H), 1.08 (dd, J=16.9, 6.8 Hz, 3H).

### Step 3: Preparation of conjugate of N-(4-(2-aminoethoxy)phenyl)-4-(((3R,4R)-1-(2-cyanoacetyl)-4-methylpiperidin-3-yl)(methyl)amino)-7H-pyrrolo[2,3-d]pyrimidine-7-carboxamide and HA

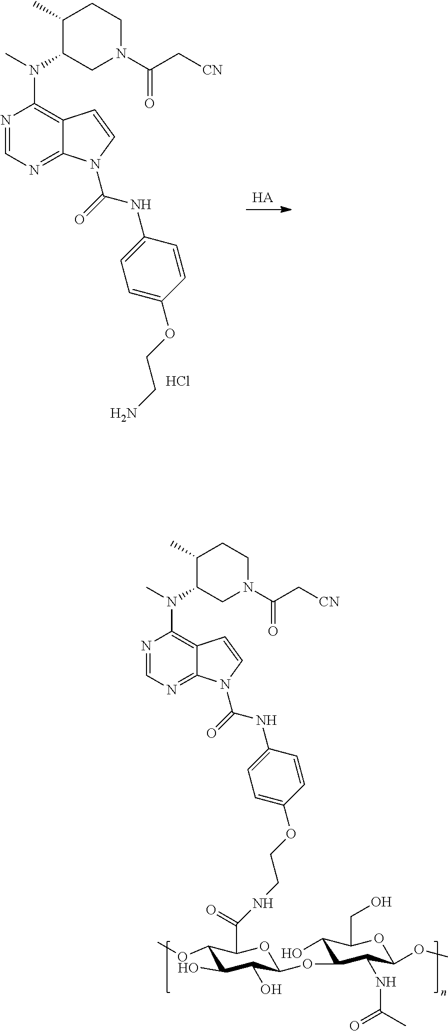

Following Step 3 of Example 1, sodium hyaluronate (MW 50 KDa, 0.153 g, 0.38 mmol, leg) and N-(4-(2-aminoethoxy)phenyl)-4-(((3R,4R)-1-(2-cyanoacetyl)-4-methyl piperidin-3-yl)(methyl)amino)-7H-pyrrolo[2,3-d]pyrimidine-7-carboxamide hydrochloride (0.2 g, 0.38 mmol, 1 eq) afforded the title compound (0.22 g, yield: 66%, DSR=11%); ¹H-NMR (400 MHz, D₂O) δ ppm 8.0-6.6 (m, 0.77H), 4.53 (br, 1.58H), 4.01-3.19 (m, 12H), 2.77-2.58 (m, 0.11H), 1.95 (s, 3H), 1.28 (br, 0.22H), 1.15-0.95 (m, 0.33H).

With Step 3 of Example 1, reaction of sodium hyaluronate (MW 2000 KDa) provided corresponding product (0.24 g, yield: 73%, DSR=10%).

### Example 15

### Preparation of conjugate of N-(4-(4-aminobutoxy)phenyl)-4-(((3R,4R)-1-(2-cyanoacetyl)-4-methylpiperidin-3-yl)(methyl)amino)-7H-pyrrolo [2,3-d]pyrimidine-7-carboxamide and HA

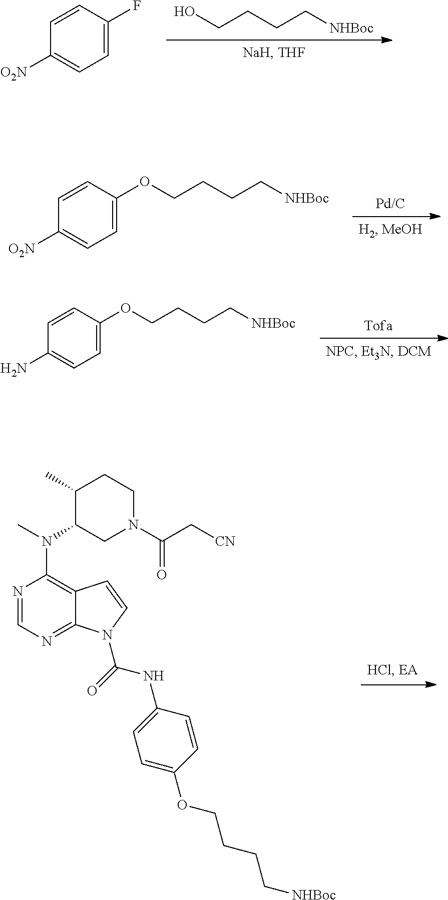

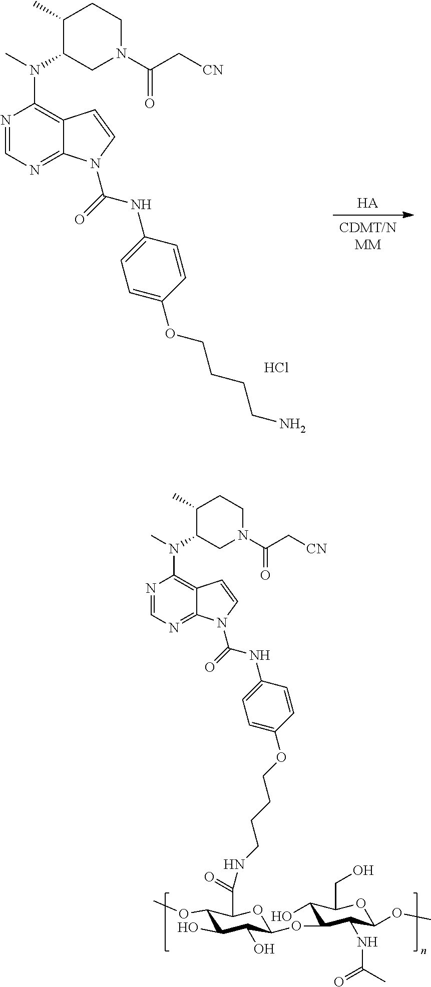

### Step 1: Preparation of tert-butyl(4-(4-nitrophenoxy)butyl)carbamate

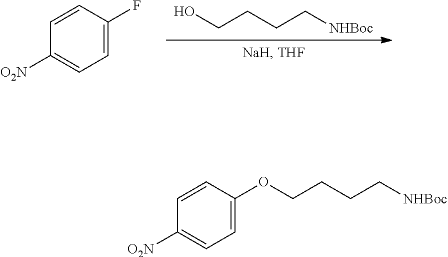

To a solution of tert-butyl (4-hydroxybutyl)carbamate (3.7 g, 19.5 mmol, 1.1 eq) in THF (40 mL) was added NaH (1.772 g, 44.3 mmol, 2.5 eq) at 0° C. under N₂. The reaction mixture was stirred at 0° C. for 30 min, and then 1-fluoro-4-nitrobenzene (2.5 g, 17.72 mmol, 1 eq) was added to. The resulting mixture was refluxed for 12 h. After most of 1-fluoro-4-nitrobenzene was consumed, the reaction was quenched by saturated NH4Cl solution in water (100 mL) and extracted by ethyl acetate (40 mL*2). The combine organic phase was washed with saturated NaCl solution in water (100 mL), dried over anhydrous Na₂SO₄ and concentrated in high vacuum. The residue was purified by silical gel chromatography to give the title product (4 g, yield: 73%); MS (m/z): [M+H]+ calcd for C₁₅H₂₂N₂O₅, 311.15; found, 311.1.

### Step 2: Preparation of tert-butyl(4-(4-aminophenoxy)butyl)carbamate

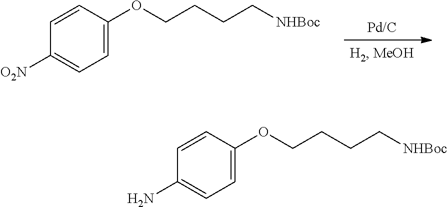

To a solution of tert-butyl (4-(4-nitrophenoxy)butyl)carbamate (2 g, 6.444 mmol, 1 eq) in methanol (30 mL) was added 10% Pd/C (0.2 g), the reaction mixture was stirred under H2 balloon for 24 h at room temperature. After tert-butyl (4-(4-nitrophenoxy)butyl)carbamate was completely consumed, the reaction mixture was filtered through a pad of Celite and the pad was washed with methanol (10 mL*2). The combined filtrates was concentrated to give the title product (1.8 g, yield: 100%); MS (m/z): [M+H]+ calcd for C₁₅H₂₄N₂O₃, 281.18; found, 281.1.

### Step 3: Preparation of tert-butyl (4-(4-(4-(((3R,4R)-1-(2-cyanoacetyl)-4-methylpiperidin-3-yl)(methyl)amino)-7H-pyrrolo[2,3-d]pyrimidine-7-carboxamido)phenoxy)butyl)carbamate

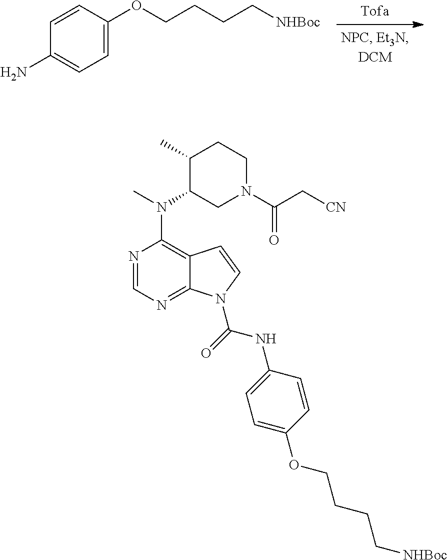 [SMILES: CC(C)(C)OC(=O)NCCCCOc1ccc(N)cc1 ; C[C@@H]1CCN(C(=O)CC#N)C[C@@H]1N(C)c1ncnc2c1ccn2C(=O)Nc1ccc(OCCCCNC(=O)OC(C)(C)C)cc1]

Following Step 1 of Example 1, tofacitinib (2.23 g, 7.133 mmol, 1 eq) and tert-butyl (4-(4-aminophenoxy)butyl)carbamate (2 g, 7.133 mmol, 1 eq) gave the title product (2.6 g, yield: 59%); MS (m/z): [M+H]+ calcd for C₃₂H₄₂N₈O₅, 619.33; found, 619.2.

### Step 4: Preparation of N-(4-(4-aminobutoxy)phenyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride

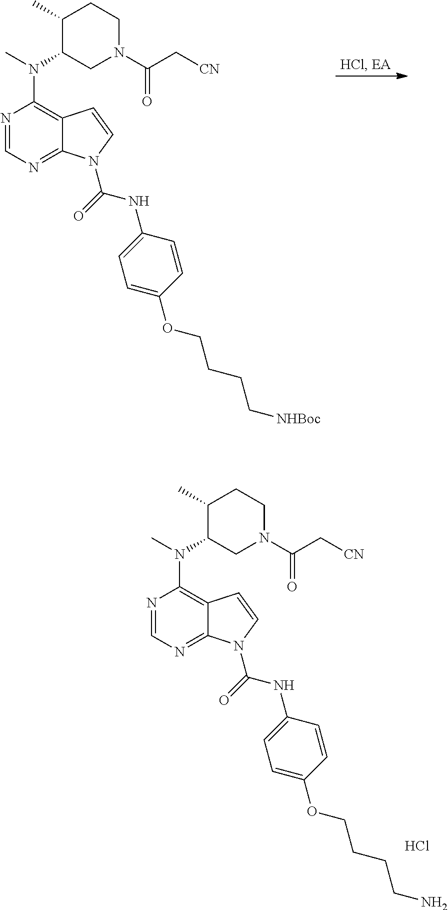 [SMILES: C[C@@H]1CCN(C(=O)CC#N)C[C@@H]1N(C)c1ncnc2c1ccn2C(=O)Nc1ccc(OCCCCNC(=O)OC(C)(C)C)cc1 ; "HCl, EA" ; C[C@@H]1CCN(C(=O)CC#N)C[C@@H]1N(C)c1ncnc2c1ccn2C(=O)Nc1ccc(OCCCCN)cc1 ; Cl]

Following Step 2 of Example 1, tert-butyl (4-(4-(4-(((3R,4R)-1-(2-cyanoacetyl)-4-methylpiperidin-3-yl)(methyl)amino)-7H-pyrrolo[2,3-d]pyrimidine-7-carboxamido)phenoxy)butyl) carbamate (1.3 g, 2.1 mmol, 1 eq) gave the desired product as HCl salt (1.1 g, yield: 100%); MS (m/z): [M+H]+ calcd for C₂₇H₃₄N₈O₃, 519.28; found, 519.2. ¹H-NMR (400 MHz, D₂O) δ ppm 8.14-8.11 (d, J=10.8 Hz, 1H), 7.43-7.21 (m, 3H), 6.83 (d, J=8.6 Hz, 2H), 6.70-6.45 (m, 1H), 4.49 (br, 1H), 4.12-3.09 (m, 13H), 2.36 (m, 1H), 1.85-1.55 (m, 6H), 1.04 (d, J=7.4 Hz, 3H).

### Step 5: Preparation of conjugate of N-(4-(4-aminobutoxy)phenyl)-4-(((3R,4R)-1-(2-cyanoacetyl)-4-methylpiperidin-3-yl)(methyl)amino)-7H-pyrrolo[2,3-d]pyrimidine-7-carboxamide and HA

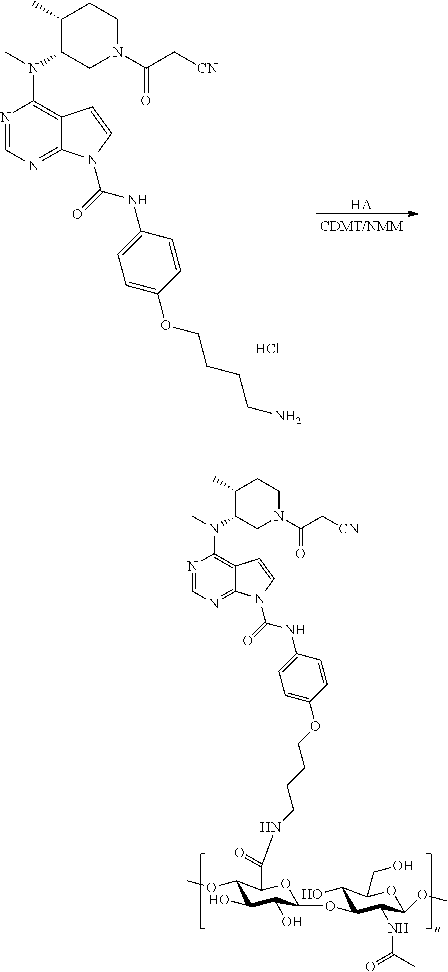

Following Step 3 of Example 1, sodium hyaluronate (MW 50 KDa, 0.153 g, 0.38 mmol, 1 eq) and N-(4-(4-aminobutoxy)phenyl)-4-(((3R,4R)-1-(2-cyanoacetyl)-4-methylpiperidin-3-yl)(methyl)amino)-7H-pyrrolo[2,3-d] pyrimidine-7-carboxamide hydrochloride (00.2 g, 0.38 mmol, 1 eq) afforded the title compound (0.22 g, yield: 66%, DSR=31%); ¹H-NMR (400 MHz, D₂O) δ ppm 8.60-5.22 (m, 2.17H), 4.7-4.2 (m, 3.1H), 4.0-3.25 (m, 12H), 3.3-2.9 (m, 1.24H), 2.84-2.11 (m, 0.31H), 2.02 (s, 3H), 1.79 (br, 1.24H), 1.30 (br, 0.62H), 1.15-0.5 (m, 0.93H).

With Step 3 of Example 1, reaction of sodium hyaluronate (MW 2000 KDa) provided corresponding product (0.123 g, yield: 36%, DSR=4%).

### Example 16

### Preparation of conjugate of N-(4-(2-(2-aminoethoxy)ethoxy)phenyl)-4-(((3R,4R)-1-(2-cyanoacetyl)-4-methylpiperidin-3-yl)(methyl)amino)-7H-pyrrolo[2,3-d]pyrimidine-7-carboxamide and HA

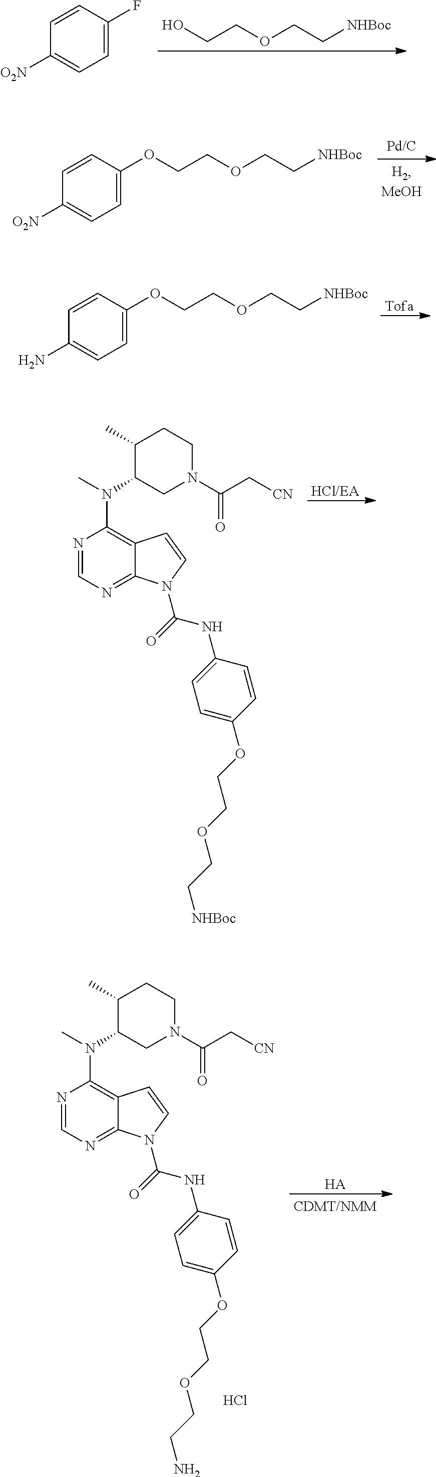

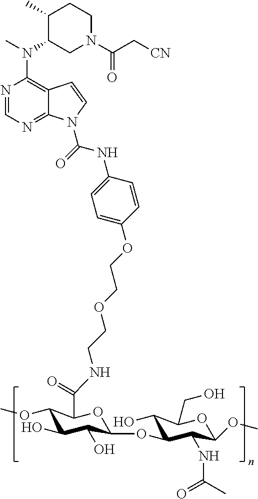

### Step 1: Preparation of tert-butyl (2-(2-(4-nitrophenoxy)ethoxy)ethyl)carbamate

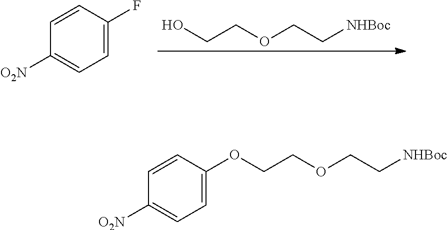

To a solution of tert-butyl (2-(2-hydroxyethoxy)ethyl)carbamate (3.2 g, 15.59 mmol, 1.1 eq) in THF (40 mL) was added NaH (1.42 g, 35.425 mmol, 2.5 eq) at 0° C. under N₂, the reaction mixture was stirred at 0° C. for 30 min, then 1-fluoro-4-nitrobenzene (2 g, 14.17 mmol, 1 eq) was added-into, the resulting mixture was refluxed for 12 h. After most of 1-fluoro-4-nitrobenzene was consumed, the reaction was quenched by saturated NH₄Cl solution in water (100 mL) and extracted by ethyl acetate (50 mL*2). The combine organic phase was washed with saturated NaCl solution in water (100 mL), dried over anhydrous Na₂SO₄ and concentrated in high vacuum. The residue was purified by silical gel chromatography to give the title product (2 g, yield: 43.3%); MS (m/z): [M+H]+ calcd for C₁₅H₂₂N₂O₆, 327.15; found, 327.1.

### Step 2: Preparation of tert-butyl (2-(2-(4-aminophenoxy)ethoxy)ethyl)carbamate

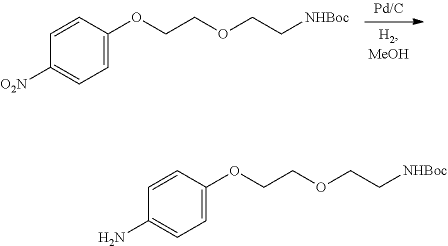 [SMILES: CC(C)(C)OC(=O)NCCOCCOc1ccc([N+](=O)[O-])cc1 ; CC(C)(C)OC(=O)NCCOCCOc1ccc(N)cc1]

To a solution of tert-butyl (2-(2-(4-nitrophenoxy)ethoxy)ethyl)carbamate (2 g, 6.13 mmol, 1 eq) in methanol (40 mL) was added 10% Pd/C (0.2 g), the reaction mixture was stirred under 1-1₂ balloon for 24 h at room temperature. After tert-butyl (2-(2-(4-nitrophenoxy)ethoxy)ethyl)carbamate was completely consumed, the reaction mixture was filtered through a pad of Celite and the pad was washed with methanol (20 mL*2). The combined filtrates was concentrated to give the title product (1.8 g, yield: 100%); MS (m/z): [M+H]+ calcd for C₁₅H₂₄N₂O₄, 297.17; found, 297.1.

### Step 3: Preparation of tert-butyl (2-(2-(4-(4-(((3R,4R)-1-(2-cyanoacetyl)-4-methylpiperidin-3-yl) (methyl)amino)-7H-pyrrolo[2,3-d]pyrimidine-7-carboxamido)phenoxy)ethoxy)ethyl)carbamate

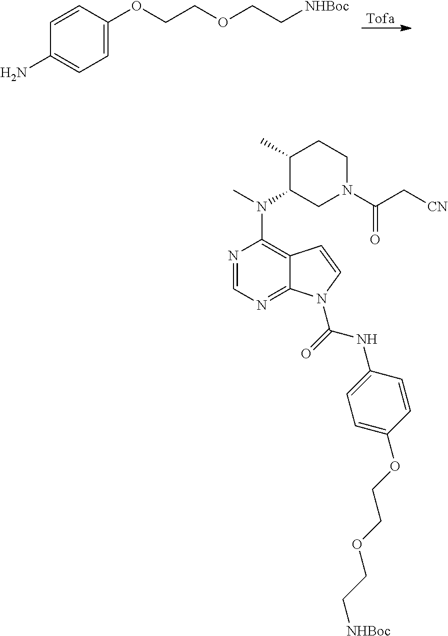 [SMILES: CC(C)(C)OC(=O)NCCOCCOc1ccc(N)cc1 ; "Tofa" ; C[C@@H]1CCN(C(=O)CC#N)C[C@@H]1N(C)c1ncnc2c1ccn2C(=O)Nc1ccc(OCCOCCNC(=O)OC(C)(C)C)cc1]

Following Step 1 of Example 1, tofacitinib (1.9 g, 6.07 mmol, 1 eq) and tert-butyl (2-(2-(4-aminophenoxy)ethoxy)ethyl) carbamate (1.8 g, 6.07 mmol, 1 eq) gave the title product (1.8 g, yield: 57%); MS (m/z): [M+H]+ calcd for C₃₂H₄₂N₈O₆, 635.32; found, 635.2.

### Step 4: Preparation of N-(4-(2-(2-aminoethoxy)ethoxy)phenyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride

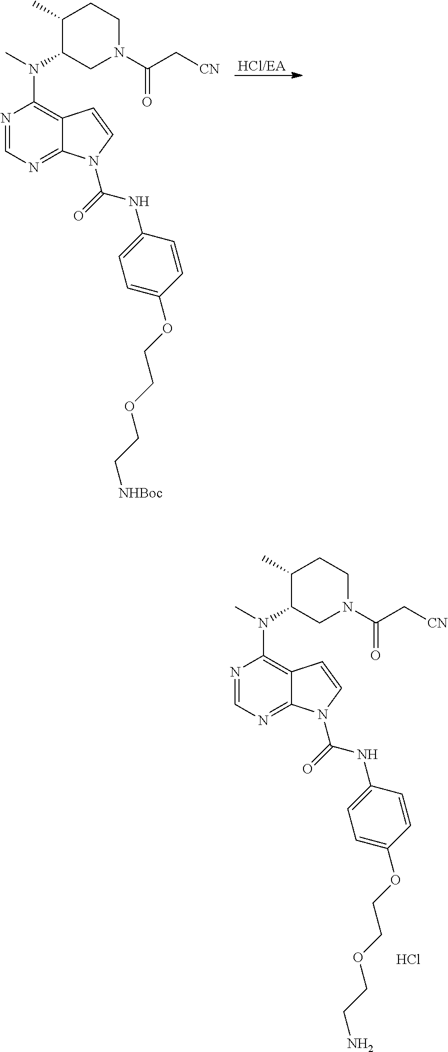

Following Step 2 of Example 1, tert-butyl (2-(2-(4-(4-(((3R,4R)-1-(2-cyanoacetyl)-4-methyl piperidin-3-yl)(methyl)amino)-7H-pyrrolo[2,3-d]pyrimidine-7-carboxamido) phenoxy)ethoxy) ethyl)carbamate (1 g, 1.575 mmol, 1 eq gave the desired product as HCl salt (0.89 g, yield: 100%); MS (m/z): [M+H]+ calcd for C₂₇H₃₄N₈O₄, 535.27; found, 535.1. ¹H-NMR (400 MHz, D₂O) δ ppm 8.19 (d, J=9.4 Hz, 1H), 7.52 (d, J=15.7 Hz, 1H), 7.37 (d, J=8.8 Hz, 2H), 6.99 (d, J=8.8 Hz, 2H), 6.72-6.50 (m, 1H), 4.63-4.54 (m, 1H), 4.23 (s, 2H), 4.09-3.81 (m, 7H), 3.74-3.12 (m, 8H), 2.42 (s, 1H), 1.77 (m, 2H), 1.08 (d, J=6.8 Hz, 3H).

### Step 5: Preparation of conjugate of N-(4-(2-(2-aminoethoxy)ethoxy)phenyl)-4-(((3R,4R)-1-(2-cyanoacetyl)-4-methylpiperidin-3-yl)(methyl)amino)-7H-pyrrolo[2,3-d]pyrimidine-7-carboxamide and HA

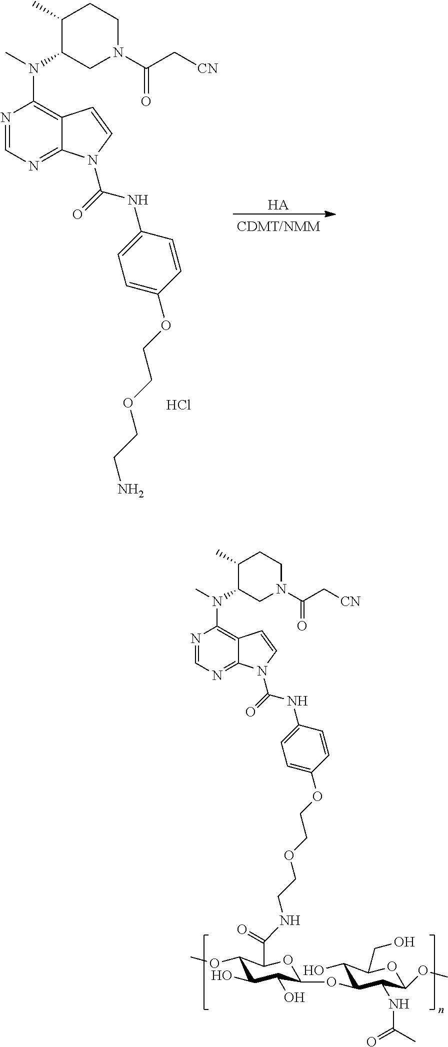

Following Step 3 of Example 1, sodium hyaluronate (MW 50 KDa, 0.141 g, 0.35 mmol, 1 eq) and N-(4-(2-(2-aminoethoxy)ethoxy)phenyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride (0.2 g, 0.35 mmol, 1 eq) afforded the title compound (0.15 g, yield: 32%, DSR=25%); ¹H-NMR (400 MHz, D₂O) δ ppm 8.49-7.25 (m, 0.76H), 7.25-5.75 (m, 1.0H), 4.78-4.02 (m, 3.23H), 4.02-3.22 (m, 12H), 3.2-2.75 (m, 1.23H), 2.5 (br, 0.25H), 2.01 (s, 3H), 1.63-1.16 (m, 0.5H), 1.25-0.5 (m, 0.75H).

With Step 3 of Example 1, reaction of sodium hyaluronate (MW 2000 KDa) provided corresponding product (0.132 g, yield: 42%, DSR=28%).

### Example 17

### Preparation of conjugate of 3-((3R,4R)-4-methyl-3-(methyl(7-(piperazine-1-carbonyl)-7H-pyrrolo[2,3-d]pyrimidin-4-yl)amino)piperidin-1-yl)-3-oxopropanenitrile and HA

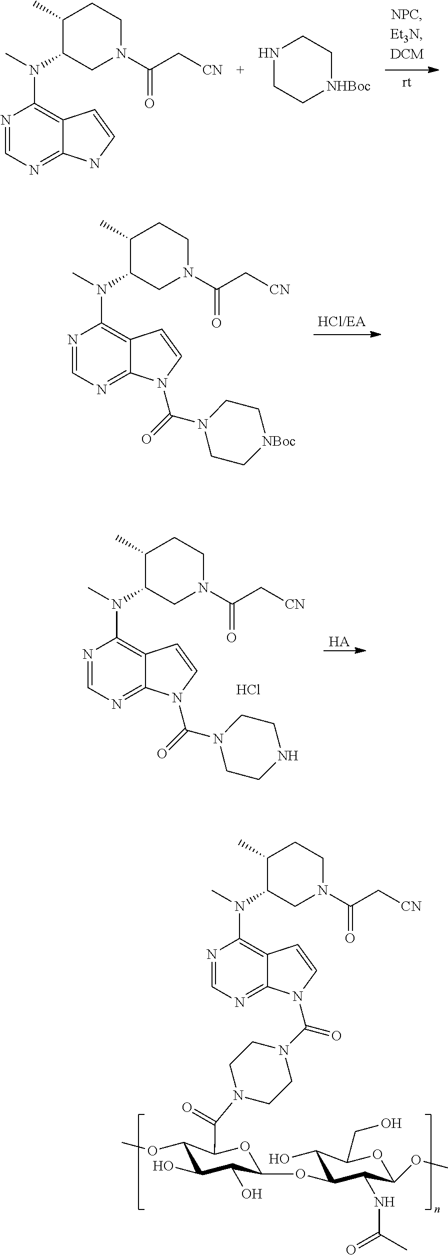

### Step 1: Preparation of tert-butyl 4-(4-(((3R,4R)-1-(2-cyanoacetyl)-4-methylpiperidin-3-yl)(methyl)amino)-7H-pyrrolo[2,3-d]pyrimidine-7-carbonyl)piperazine-1-carboxylate

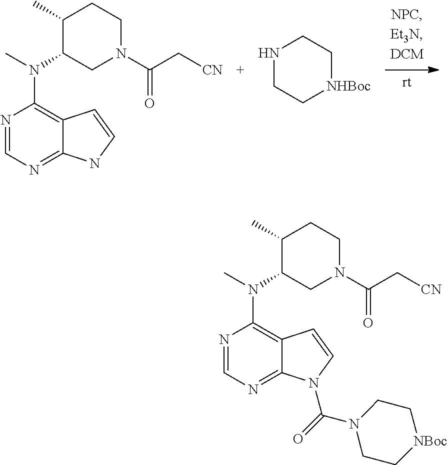

Following Step 1 of Example 1, tofacitinib (1.68 g, 5.37 mmol, 1 eq) and tert-butyl piperazine-1-carboxylate (1 g, 5.37 mmol, 1 eq) gave the title product (0.9 g, yield: 32%); MS (m/z): [M+H]±calcd for C₂₆H₃₆N₈O₄, 525.29; found, 525.2.

### Step 2: Preparation of 3-((3R,4R)-4-methyl-3-(methyl(7-(piperazine-1-carbonyl)-7H-pyrrolo[2,3-d]pyrimidin-4-yl)amino)piperidin-1-yl)-3-oxopropanenitrile hydrochloride

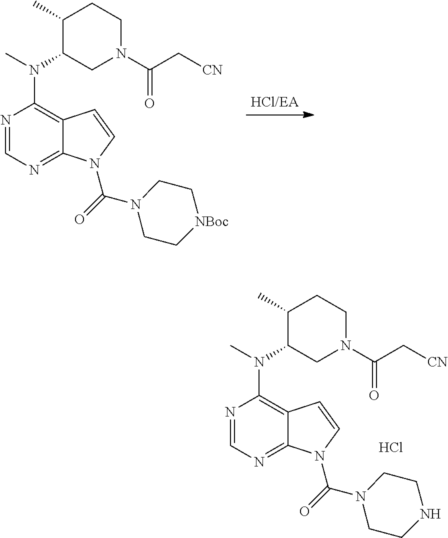 [SMILES: C[C@@H]1CCN(C(=O)CC#N)C[C@@H]1N(C)c1ncnc2c1ccn2C(=O)N1CCN(C(=O)OC(C)(C)C)CC1 ; C[C@@H]1CCN(C(=O)CC#N)C[C@@H]1N(C)c1ncnc2c1ccn2C(=O)N1CCNCC1 ; Cl]

Following Step 2 of Example 1, tert-butyl 4-(4-(((3R,4R)-1-(2-cyanoacetyl)-4-methyl piperidin-3-yl)(methyl)amino)-7H-pyrrolo[2,3-d]pyrimidine-7-carbonyl)piperazine-1-carboxylate (0.8 g, 1.525 mmol, 1 eq) gave the desired product as HCl salt (0.7 g, yield: 100%); MS (m/z): [M+H]+ calcd for C₂₁H₂₈N₈O₂, 425.23; found, 425.1. ¹H-NMR (400 MHz, D₂O) δ ppm 8.41 (d, J=5.5 Hz, 1H), 7.59 (d, J=3.9 Hz, 1H), 7.04 (s, 1H), 4.74-4.57 (m, 1H), 4.17-3.82 (m, 7H), 3.80-3.06 (m, 10H), 2.68-2.51 (m, 1H), 2.06-1.90 (m, 1H), 1.89-1.71 (m, 1H), 1.16 (dd, J=14.8, 7.1 Hz, 3H).

### Step 3: Preparation of conjugate of 3-((3R,4R)-4-methyl-3-(methyl(7-(piperazine-1-carbonyl)-7H-pyrrolo[2,3-d]pyrimidin-4-yl)amino)piperidin-1-yl)-3-oxopropanenitrile and HA

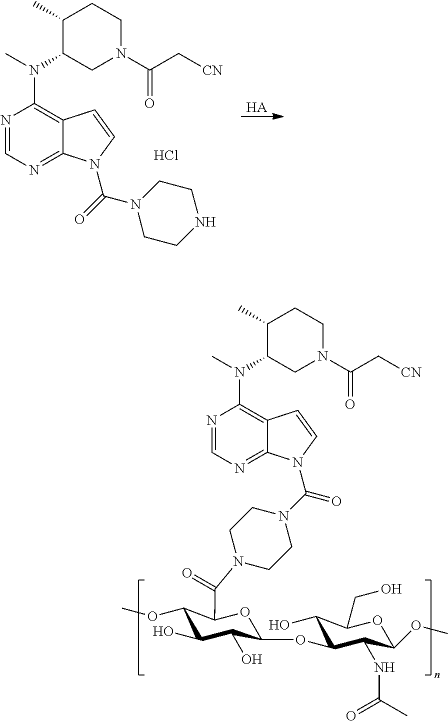

Following Step 3 of Example 1, sodium hyaluronate (MW 50 KDa, 0.175 g, 0.434 mmol, 1 eq) and N-(4-aminobutyl)-4-(((3R,4R)-1-(2-cyanoacetyl)-4-methylpiperidin-3-yl) (methyl)amino)-7H-pyrrolo[2,3-d]pyrimidine-7-carboxamide hydrochloride (0.2 g, 0.434 mmol, 1 eq) afforded the title compound (0.2 g, yield: 57.3%, DSR=24%); ¹H-NMR (400 MHz, D₂O) δ ppm 8.21 (br, 0.24H), 7.33 (br, 0.24H), 6.91 (br, 0.24H), 4.7-4.3 (m, 2.32H), 4.18-3.12 (m, 14H), 2.46 (br, 0.24H), 2.02 (s, 3H), 1.79 (br, 0.24H), 1.30 (m, 0.24H), 1.18-0.93 (m, 0.72H).

With Step 3 of Example 1, reaction of sodium hyaluronate (MW 2000 KDa) provided corresponding product (0.162 g, yield: 46.4%, DSR=28%).

### Example 18

### Preparation of conjugate of 3-((3R,4R)-4-methyl-3-(methyl(7-(piperazine-1-carbonyl)-7H-pyrrolo[2,3-d]pyrimidin-4-yl)amino)piperidin-1-yl)-3-oxopropanenitrile and HA

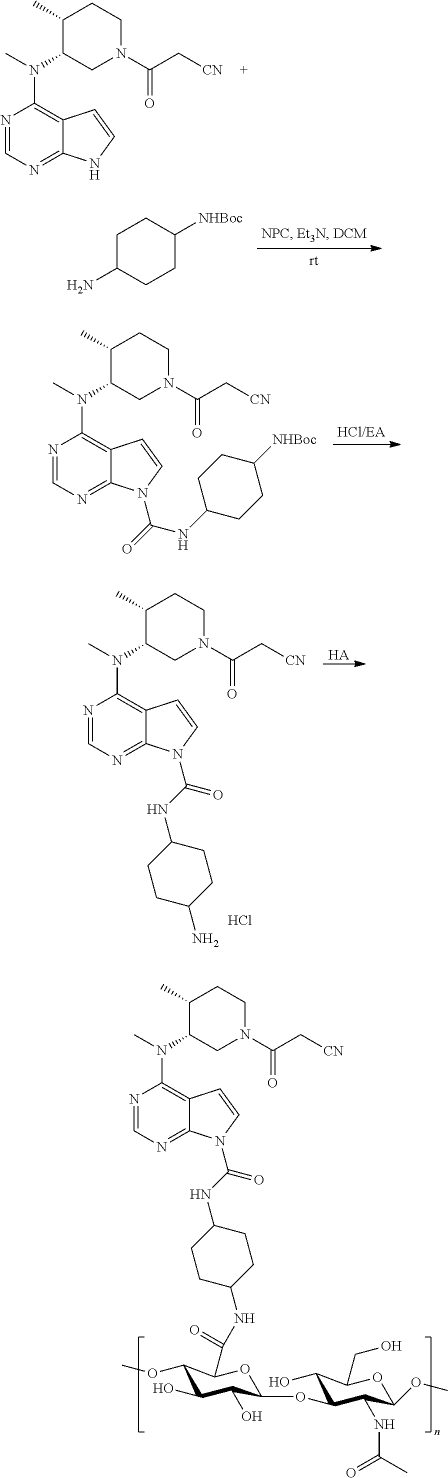

### Step 1: Preparation of tert-butyl (4-(4-(((3R,4R)-1-(2-cyanoacetyl)-4-methylpiperidin-3-yl)(methyl)amino)-7H-pyrrolo[2,3-d]pyrimidine-7-carboxamido)cyclohexyl)carbamate

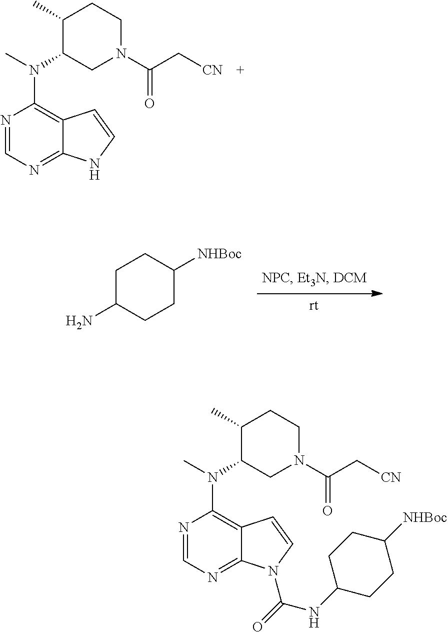 [SMILES: C[C@@H]1CCN(C(=O)CC#N)C[C@@H]1N(C)c1ncnc2[nH]ccc12 ; CC(C)(C)OC(=O)NC1CCC(N)CC1 ; C[C@@H]1CCN(C(=O)CC#N)C[C@@H]1N(C)c1ncnc2c1ccn2C(=O)NC1CCC(NC(=O)OC(C)(C)C)CC1]

Following Step 1 of Example 1, tofacitinib (1.895 g, 6.066 mmol, 1 eq) and tert-butyl (4-aminocyclohexyl) carbamate (1.3 g, 6.066 mmol, 1 eq) gave the title product (1.785 g, yield: 53.2%); MS (m/z): [M+H]+ calcd for C₂₈H₄₀N₈O₄, 553.32; found, 553.2.

### Step 2: Preparation of N-(4-aminocyclohexyl)-4-(((3R,4R)-1-(2-cyanoacetyl)-4-methylpiperidin-3-yl)(methyl)amino)-7H-pyrrolo[2,3-d]pyrimidine-7-carboxamidehydrochloride

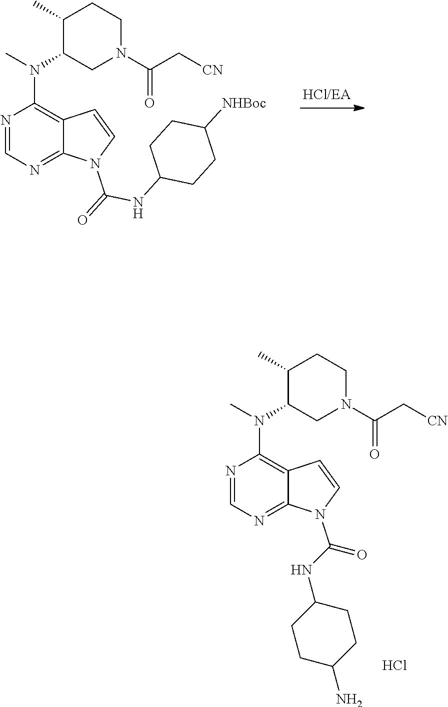 [SMILES: C[C@@H]1CCN(C(=O)CC#N)C[C@@H]1N(C)c1ncnc2c1ccn2C(=O)NC1CCC(NC(=O)OC(C)(C)C)CC1 ; C[C@@H]1CCN(C(=O)CC#N)C[C@@H]1N(C)c1ncnc2c1ccn2C(=O)NC1CCC(N)CC1 ; Cl]

Following Step 2 of Example 1, tert-butyl (4-(4-(((3R,4R)-1-(2-cyanoacetyl)-4-methylpiperidin-3-yl)(methyl)amino)-7H-pyrrolo[2,3-d]pyrimidine-7-carboxamido)cyclohexyl) carbamate (1.5 g, 2.71 mmol, leg) gave the desired product as HCl salt (1 g, yield: 77%); MS (m/z): [M+H]±calcd for C₂₃H₃₂N₈O₂, 453.26; found, 453.2. ¹H-NMR (400 MHz, D₂O) δ ppm 8.6-8.2 (m, 1H), 7.90-7.70 (m, 1H), 6.89 (br, 1H), 4.69-4.59 (m, 1H), 4.14-3.13 (m, 11H), 2.62-2.49 (m, 1H), 2.31-2.11 (m, 1H), 2.05-1.58 (m, 9H), 1.15 (dd, J=6.6, 5.4 Hz, 3H).

### Step 3: Preparation of conjugate of 3-((3R,4R)-4-methyl-3-(methyl(7-(piperazine-1-carbonyl)-7H-pyrrolo[2,3-d]pyrimidin-4-yl)amino)piperidin-1-yl)-3-oxopropanenitrile and HA

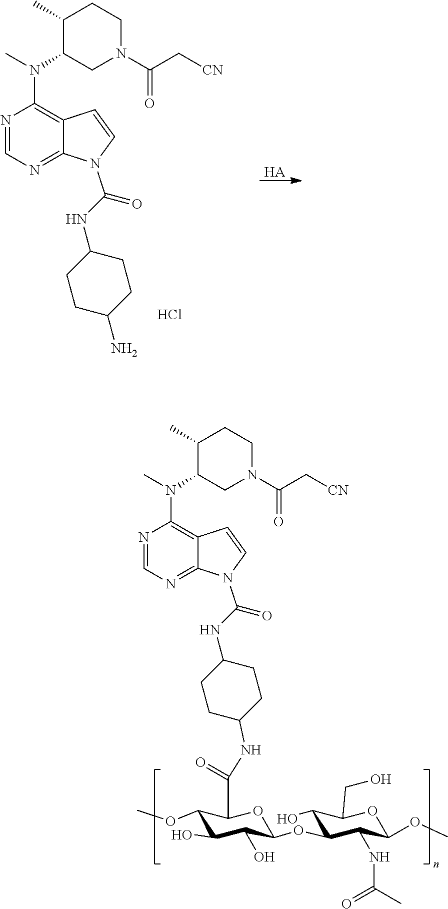

Following Step 3 of Example 1, sodium hyaluronate (MW 50 KDa, 0.178 g, 0.44 mmol, 1 eq) and 3-((3R,4R)-4-methyl-3-(methyl(7-(piperazine-1-carbonyl)-7H-pyrrolo[2,3-d] pyrimidin-4-yl)amino)piperidin-1-yl)-3-oxopropanenitrile hydrochloride (0.2 g, 0.44 mmol, 1 eq) afforded the title compound (0.2 g, yield: 55%, DSR=25%); ¹H-NMR (400 MHz, D₂O) δ ppm 8.4-7.8 (m, 0.25H), 7.7-7.2 (m, 0.25H), 6.90-6.0 (m, 0.25H), 4.45 (br, 2.5H), 3.77-3.27 (m, 12H), 3.17-3.06 (m, 0.51H), 2.42 (br, 0.25H), 1.96 (s, 3H), 1.85-1.52 (m, 2H), 1.24 (br, 0.5H), 1.1-0.9 (m, 0.75H).

With Step 3 of Example 1, reaction of sodium hyaluronate (MW 2000 KDa) provided corresponding product (0.109 g, yield: 30%, DSR=9%).

### Example 19

### Preparation of conjugate of N-(2-aminocyclohexyl)-4-(((3R,4R)-1-(2-cyanoacetyl)-4-methylpiperidin-3-yl)(methyl)amino)-7H-pyrrolo[2,3-d]pyrimidine-7-carboxamide and HA

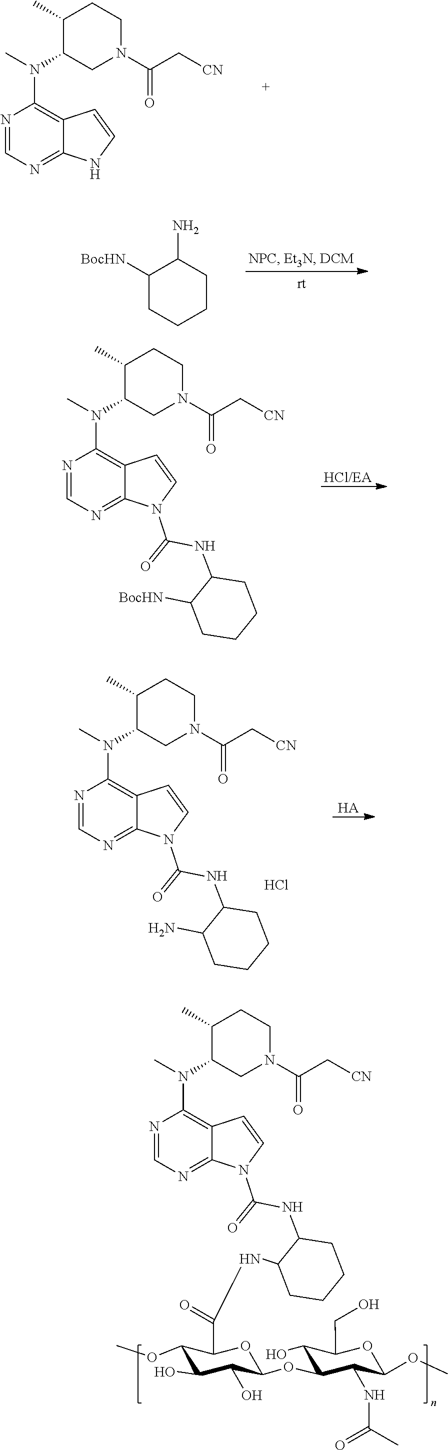

### Step 1: Preparation of tert-butyl (2-(4-(((3R,4R)-1-(2-cyanoacetyl)-4-methylpiperidin-3-yl)(methyl)amino)-7H-pyrrolo[2,3-d]pyrimidine-7-carboxamido)cyclohexyl)carbamate

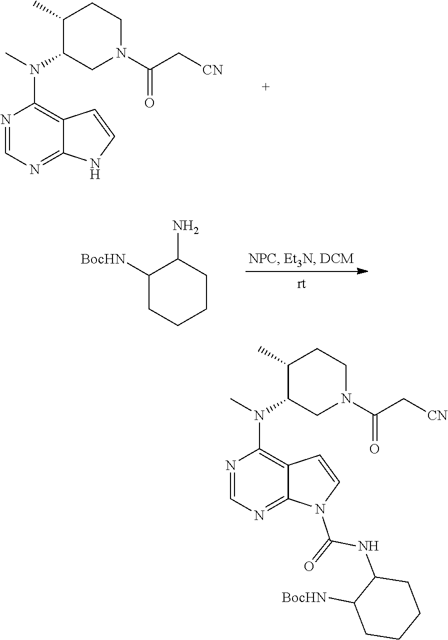

Following Step 1 of Example 1, tofacitinib (1.46 g, 4.67 mmol, 1 eq) and tert-butyl (2-aminocyclohexyl)carbamate (1 g, 4.67 mmol, 1 eq) gave the title product (2 g, yield: 78%); MS (m/z): [M+H]±calcd for C₂₈H₄₀N₈O₄, 553.32; found, 553.2.

### Step 2: Preparation of N-(2-aminocyclohexyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride

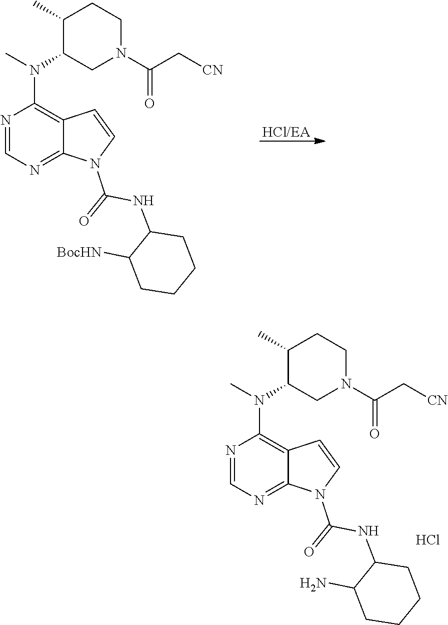 [SMILES: C[C@@H]1CCN(C(=O)CC#N)C[C@@H]1N(C)c1ncnc2c1ccn2C(=O)NC1CCCCC1NC(=O)OC(C)(C)C ; "HCl/EA" ; C[C@@H]1CCN(C(=O)CC#N)C[C@@H]1N(C)c1ncnc2c1ccn2C(=O)NC1CCCCC1N ; Cl]

Following Step 2 of Example 1, tert-butyl (2-(4-(((3R,4R)-1-(2-cyanoacetyl)-4-methylpiperidin-3-yl)(methyl)amino)-7H-pyrrolo[2,3-d]pyrimidine-7-carboxamido)cyclohexyl)carbamate (1.3 g, 2.352 mmol, 1 eq) gave the desired product as HCl salt (1.15 g, yield: 100%); MS (m/z): [M+H]+ calcd for C₂₃H₃₂N₈O₂, 453.26; found, 453.2. ¹H-NMR (400 MHz, D₂O) δ ppm 8.52-8.36 (m, 1H), 7.90-7.78 (m, 1H), 6.93 (s, 1H), 4.63 (m, 2H), 4.21-3.78 (m, 4H), 3.70-3.25 (m, 6H), 2.55 (br, 1H), 2.14 (m, 1H), 2.04-1.31 (m, 9H), 1.20-1.04 (m, 3H).

### Step 3: Preparation of conjugate of N-(2-aminocyclohexyl)-4-(((3R,4R)-1-(2-cyanoacetyl)-4-methylpiperidin-3-yl)(methyl)amino)-7H-pyrrolo[2,3-d]pyrimidine-7-carboxamide and HA

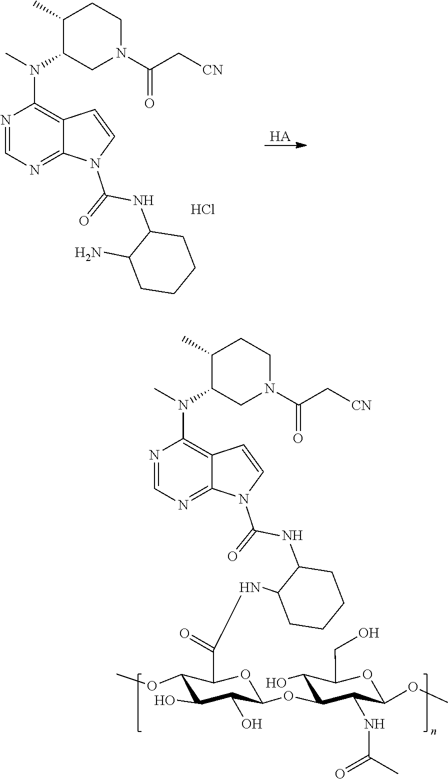

Following Step 3 of Example 1, sodium hyaluronate (MW 50 KDa, 0.161 g, 0.4 mmol, 1 eq) and N-(2-aminocyclohexyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride (0.2 g, 0.4 mmol, 1 eq) afforded the title compound (0.2 g, yield: 59%, DSR=18%); ¹H-NMR (400 MHz, D₂O) δ ppm 8.62-7.85 (m, 0.18H), 7.82-7.41 (m, 0.18H), 6.86-6.70 (m, 0.18H), 4.7-4.2 (m, 2H), 4.23-2.54 (m, 12.16H), 2.42 (br, 0.18H), 2.00 (s, 3H), 1.50-1.2 (m, 0.86H), 1.22-0.87 (m, 1.05H).

With Step 3 of Example 1, reaction of sodium hyaluronate (MW 2000 KDa) provided corresponding product (0.224 g, yield: 67%, DSR=7%).

### Example 20

### Preparation of conjugate of 4-(((3R,4R)-1-(2-cyanoacetyl)-4-methyl piperidin-3-yl)(methyl)amino)-N′-ethyl-7H-pyrrolo[2,3-d]pyrimidine-7-carbohydrazide and HA

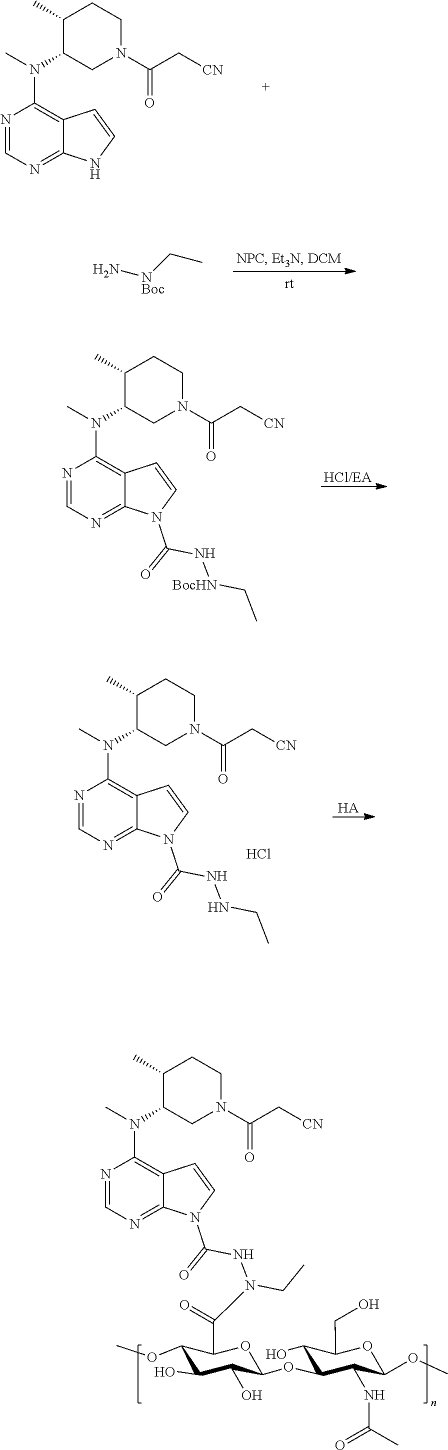

### Step 1: Preparation of tert-butyl 2-(4-(((3R,4R)-1-(2-cyanoacetyl)-4-methylpiperidin-3-yl)(methyl)amino)-7H-pyrrolo[2,3-d]pyrimidine-7-carbonyl)-1-ethylhydrazine-1-carboxylate

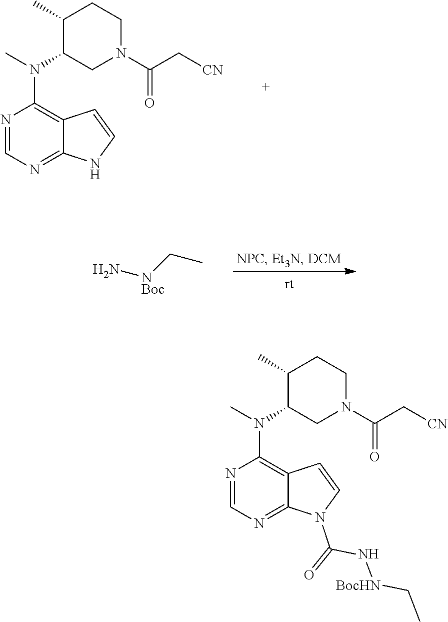

Following Step 1 of Example 1, tofacitinib (2.5 g, 8.114 mmol, 1 eq) and tert-butyl 1-ethylhydrazine-1-carboxylate (1.3 g, 8.114 mmol, 1 eq) gave the title product (1.745 g, yield: 43%); MS (m/z): [M+H]+ calcd for C₂₄H₃₄N₈O₄, 499.27; found, 499.2.

### Step 2: Preparation of 4-(((3R,4R)-1-(2-cyanoacetyl)-4-methylpiperidin-3-yl)(methyl)amino)-N′-ethyl-7H-pyrrolo[2,3-d]pyrimidine-7-carbohydrazide hydrochloride

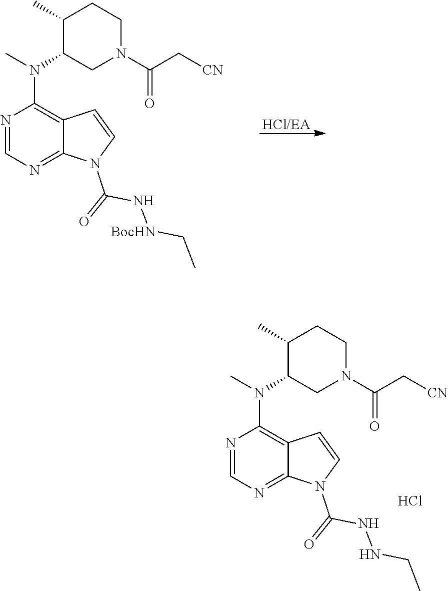

Following Step 2 of Example 1, tert-butyl 2-(4-(((3R,4R)-1-(2-cyanoacetyl)-4-methylpiperidin-3-yl)(methyl)amino)-7H-pyrrolo[2,3-d]pyrimidine-7-carbonyl)-1-ethylhydrazine-1-carboxylate (1.4 g, 2.8 mmol, 1 eq) gave the desired product as HCl salt (1 g, yield: 82%); MS (m/z): [M+H]+ calcd for C₁₉H₂₆N₈O₂, 399.22; found, 399.1. ¹H-NMR (400 MHz, D₂O) δ ppm 8.45 (d, J=4.8 Hz, 1H), 7.81 (d, J=3.6 Hz, 1H), 7.00 (s, 1H), 4.76-4.67 (m, 1H), 4.19-3.79 (m, 3H), 3.72-3.31 (m, 8H), 2.56 (br, 1H), 2.06-1.70 (m, 2H), 1.35 (br, 3H), 1.14 (dd, J=15.2, 7.1 Hz, 3H)

### Step 3: Preparation of conjugate of 4-(((3R,4R)-1-(2-cyanoacetyl)-4-methylpiperidin-3-yl)(methyl)amino)-N′-ethyl-7H-pyrrolo[2,3-d]pyrimidine-7-carbohydrazide and HA

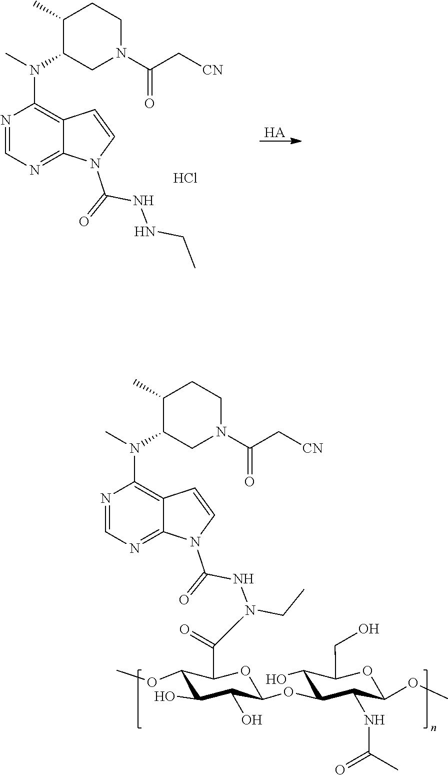

Following Step 3 of Example 1, sodium hyaluronate (MW 50 KDa, 0.186 g, 0.46 mmol, 1 eq) and 4-(((3R,4R)-1-(2-cyanoacetyl)-4-methylpiperidin-3-yl)(methyl)amino)-N′-ethyl-7H-pyrrolo[2,3-d]pyrimidine-7-carbo hydrazide hydrochloride (0.2 g, 0.46 mmol, 1 eq) afforded the title compound (0.12 g, yield: 34%, DSR=9%); ¹H-NMR (400 MHz, D₂O) δ ppm 8.4-8.3 (m, 0.09H), 7.69 (d, J=23.0 Hz, 0.09H), 6.95 (d, J=10.7 Hz, 0.09H), 4.60-4.41 (m, 2.08H), 3.84-3.34 (m, 10H), 2.47 (m, 0.09H), 2.01 (s, 3H), 1.82-1.74 (m, 0.19H), 1.31 (br, 0.27H), 1.07 (br, 0.27H).

With Step 3 of Example 1, reaction of sodium hyaluronate (MW 2000 KDa) provided corresponding product (0.198 g, yield: 55.3%, DSR=5%).

### Example 21

### Preparation of conjugate of methyl (4-(((3R,4R)-1-(2-cyanoacetyl)-4-methylpiperidin-3-yl) (methyl) amino)-7H-pyrrolo[2,3-d]pyrimidine-7-carbonyl)-L-lysinate and HA

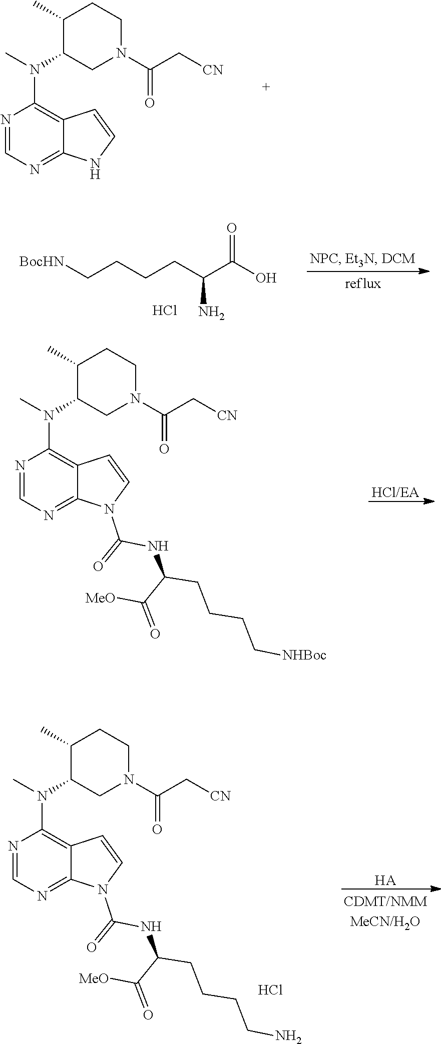

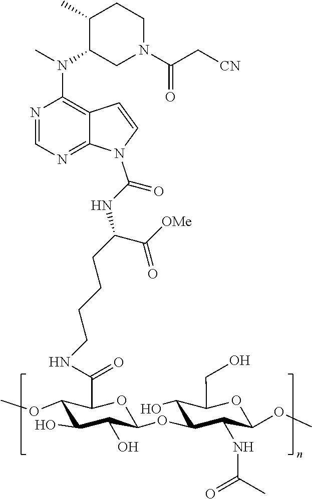

### Step 1: Preparation of methyl N6-(tert-butoxycarbonyl)-N2-(4-(((3R,4R)-1-(2-cyanoacetyl)-4-methylpiperidin-3-yl)(methyl)amino)-7H-pyrrolo[2,3-d]pyrimidine-7-carbonyl)-L-lysinate

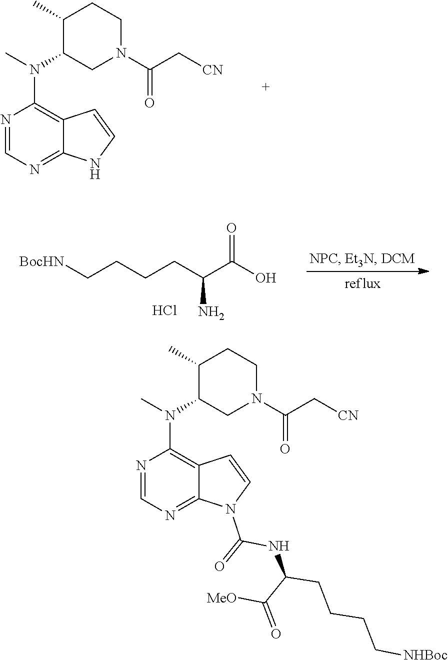

Following Step 1 of Example 1, tofacitinib (2 g, 6.4 mmol, 1 eq) and methyl N6-(tert-butoxycarbonyl)-L-lysinate hydrochloride (1.9 g, 6.4 mmol, 1 eq) gave the title product (2.2 g, yield: 57.4%); MS (m/z): [M+H]+ calcd for C₂₉H₄₂N₈O₆, 599.32; found, 599.2.

### Step 2: Preparation of methyl (4-(((3R,4R)-1-(2-cyanoacetyl)-4-methylpiperidin-3-yl)(methyl)amino)-7H-pyrrolo[2,3-d]pyrimidine-7-carbonyl)-L-lysinate hydrochloride

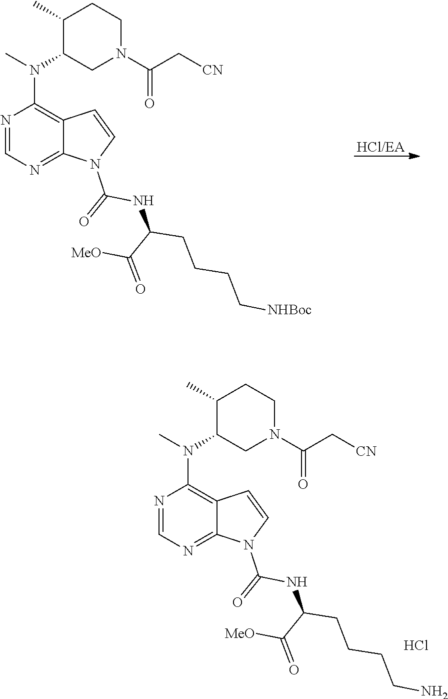 [SMILES: COC(=O)[C@H](CCCCNC(=O)OC(C)(C)C)NC(=O)n1ccc2c(N(C)[C@H]3CN(C(=O)CC#N)CC[C@H]3C)ncnc21 ; "HCl/EA" ; COC(=O)[C@H](CCCCN)NC(=O)n1ccc2c(N(C)[C@H]3CN(C(=O)CC#N)CC[C@H]3C)ncnc21 ; Cl]

Following Step 2 of Example 1, methyl N6-(tert-butoxycarbonyl)-N2-(4-(((3R,4R)-1-(2-cyanoacetyl)-4-methylpiperidin-3-yl)(methyl)amino)-7H-pyrrolo[2,3-d] pyrimidine-7-carbonyl)-L-lysinate (1.5 g, 2.5 mmol, 1 eq) gave the desired product as HCl salt (1.34 g, yield: 100%); MS (m/z): [M+H]+ calcd for C₂₄H₃₄N₈O₄, 499.27; found, 499.2. ¹H-NMR (400 MHz, D₂O) δ ppm 8.41 (d, J=4.4 Hz, 1H), 7.79 (dd, J=9.0, 4.1 Hz, 1H), 6.88 (dd, J=6.7, 2.3 Hz, 1H), 4.70-4.54 (m, 2H), 4.26-3.80 (m, 8H), 3.67-3.37 (m, 5H), 3.00 (t, J=7.6 Hz, 3H), 2.67-2.41 (m, 1H), 2.05-1.93 (m, 2H), 1.89-1.62 (m, 4H), 1.62-1.41 (m, 3H), 1.15 (dd, J=15.9, 7.1 Hz, 3H).

### Step 3: Preparation of conjugate of methyl (4-(((3R,4R)-1-(2-cyanoacetyl)-4-methylpiperidin-3-yl)(methyl)amino)-7H-pyrrolo[2,3-d]pyrimidine-7-carbonyl)-L-lysinate and HA

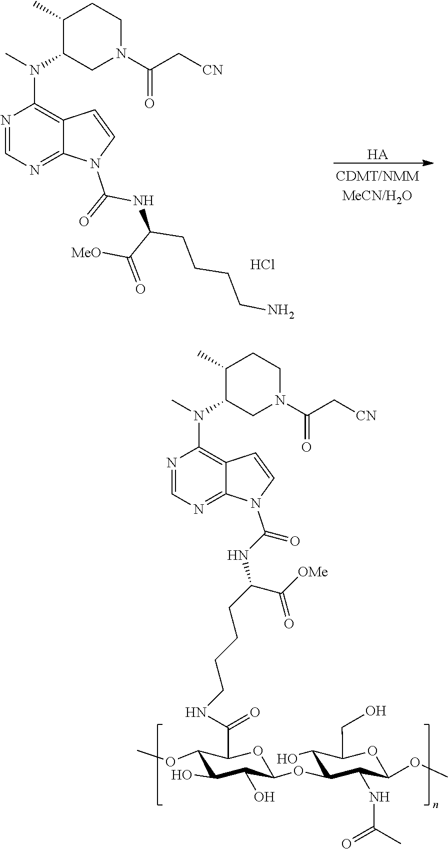

Following Step 3 of Example 1, sodium hyaluronate (MW 50 KDa, 0.151 g, 0.374 mmol, 1 eq) and methyl (4-(((3R,4R)-1-(2-cyanoacetyl)-4-methylpiperidin-3-yl)(methyl)amino)-7H-pyrrolo[2,3-d]pyrimidine-7-carbonyl)-L-lysinate hydrochloride (0.2 g, 0.374 mmol, 1 eq) afforded the title compound (0.2 g, yield: 61%, DSR=19%); ¹H-NMR (400 MHz, D₂O) δ ppm 8.25 (m, 0.2H), 7.56 (m, 0.2H), 6.72 (m, 0.2H), 4.72-2.85 (m, 15.2H), 2.45 (m, 0.4H), 2.02 (s, 3H), 1.56 (m, 1.2H), 1.31 (m, 0.2H), 1.11 (m, 0.6H). ¹H-NMR (400 MHz, D₂O) δ ppm 8.5-7.9 (m, 0.2H), 7.8-7.1 (m, 0.2H), 7.1-6.1 (m, 0.2H), 4.72-2.85 (m, 15.2H), 2.45 (br, 0.4H), 2.02 (s, 3H), 1.8-1.4 (m, 1.2H), 1.31 (br, 0.2H), 1.2-0.95 (m, 0.6H).

With Step 3 of Example 1, reaction of sodium hyaluronate (MW 2000 KDa) provided corresponding product (0.2 g, yield: 61%, DSR=23.2%).

### Example 22

### Preparation of conjugate of N-(4-aminobutyl)-4-(((3R,4R)-1-(2-cyanoacetyl)-4-methylpiperidin-3-yl)(methyl)amino)-7H-pyrrolo[2,3-d]pyrimidine-7-carbothioamide and HA

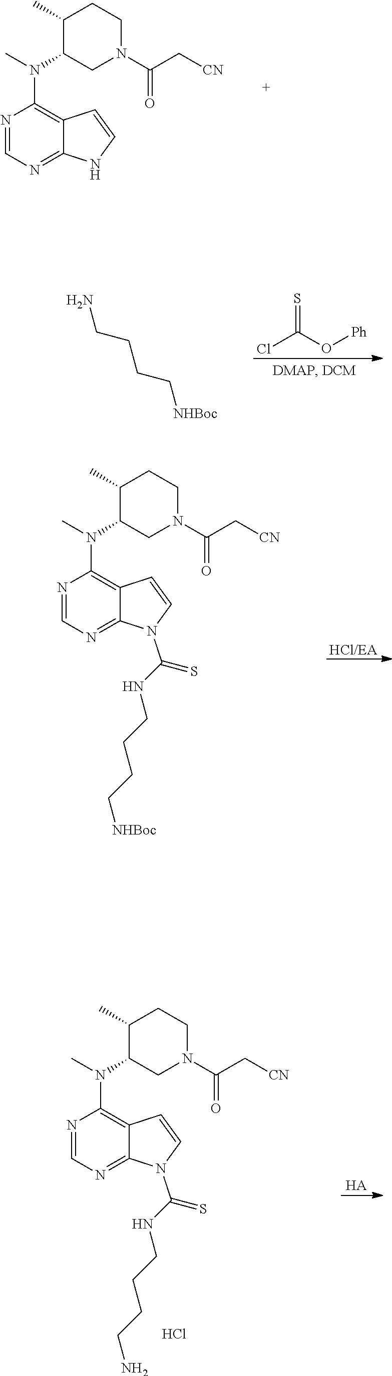

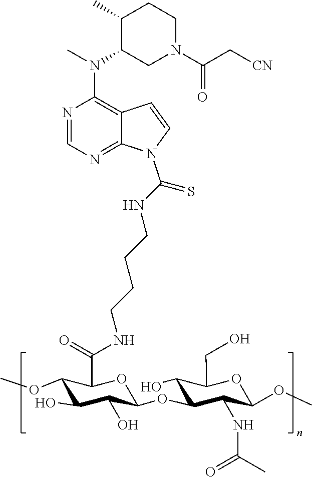

### Step 1: Preparation of tert-butyl (4-(4-(((3R,4R)-1-(2-cyanoacetyl)-4-methylpiperidin-3-yl)(methyl)amino)-7H-pyrrolo[2,3-d]pyrimidine-7-carbothioamido)butyl)carbamate

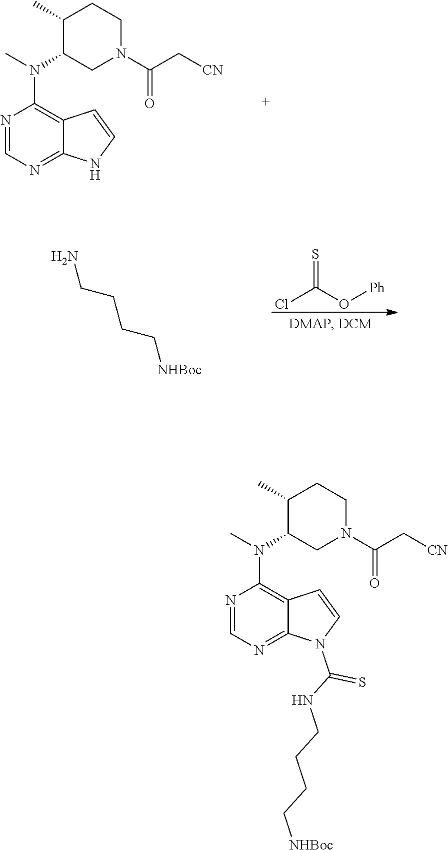 [SMILES: C[C@@H]1CCN(C(=O)CC#N)C[C@@H]1N(C)c1ncnc2[nH]ccc12 ; CC(C)(C)OC(=O)NCCCCN ; S=C(Cl)Oc1ccccc1 ; C[C@@H]1CCN(C(=O)CC#N)C[C@@H]1N(C)c1ncnc2c1ccn2C(=S)NCCCCNC(=O)OC(C)(C)C]

To a mixture of tofacitinib (2 g, 6.4 mmol, 1 eq) and O-phenyl carbonochloridothioate (2.14 g, 7.04 mmol, 1.1 eq) in dichloromethane (40 mL) was added N,N-dimethylpyridin-4-amine (2.15 g, 17.6 mmol, 2.5 eq) under N₂. The reaction mixture was heated to reflux for 3 h. Then tert-butyl (4-aminobutyl)carbamate (1.2 g, 6.4 mmol, 1 eq) was added and the mixture was refluxed for another 12 h. After most of tofacitinib was consumed, the solvent was removed under reduced pressure, the residue was purified by silical gel chromatography to give the title product (1.16 g, yield: 33.4%); MS (m/z): [M+H]+ calcd for C₂₆H₃₈N₈O₃S, 543.28; found, 543.2.

### Step 2: Preparation of N-(4-aminobutyl)-4-(((3R,4R)-1-(2-cyanoacetyl)-4-methylpiperidin-3-yl)(methyl)amino)-7H-pyrrolo[2,3-d]pyrimidine-7-carbothioamide hydrochloride

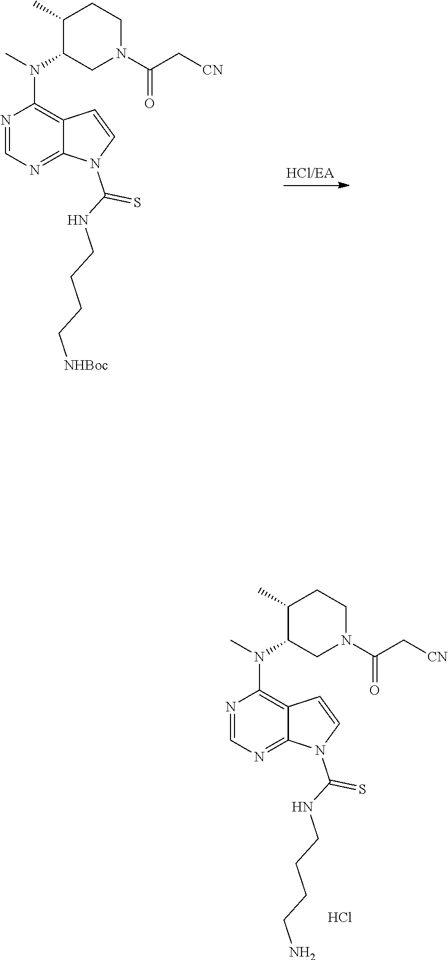 [SMILES: C[C@@H]1CCN(C(=O)CC#N)C[C@@H]1N(C)c1ncnc2c1ccn2C(=S)NCCCCNC(=O)OC(C)(C)C ; C[C@@H]1CCN(C(=O)CC#N)C[C@@H]1N(C)c1ncnc2c1ccn2C(=S)NCCCCN ; Cl]

Following Step 2 of Example 1, tert-butyl (4-(4-(((3R,4R)-1-(2-cyanoacetyl)-4-methylpiperidin-3-yl)(methyl)amino)-7H-pyrrolo[2,3-d]pyrimidine-7-carbothioamido)butyl)carbamate (1.16 g, 2.14 mmol, 1 eq) gave the desired product as HCl salt (1 g, yield: 100%); MS (m/z): [M+H]+ calcd for C₂₁H₃₀N₈OS, 443.23; found, 443.2. ¹H-NMR (400 MHz, D₂O) δ ppm 8.36 (d, J=36.1 Hz, 2H), 6.92 (s, 1H), 4.63 (d, J=1.2 Hz, 1H), 4.1-3.75 (m, 4H), 3.39 (s, 3H), 3.23 (s, 4H), 2.96 (t, J=7.5 Hz, 3H), 2.47 (s, 1H), 2.0-1.6 (m, 5H), 1.23-1.06 (m, 4H).

### Step 3: Preparation of conjugate of N-(4-aminobutyl)-4-(((3R,4R)-1-(2-cyanoacetyl)-4-methylpiperidin-3-yl)(methyl)amino)-7H-pyrrolo[2,3-d]pyrimidine-7-carbothioamide and HA

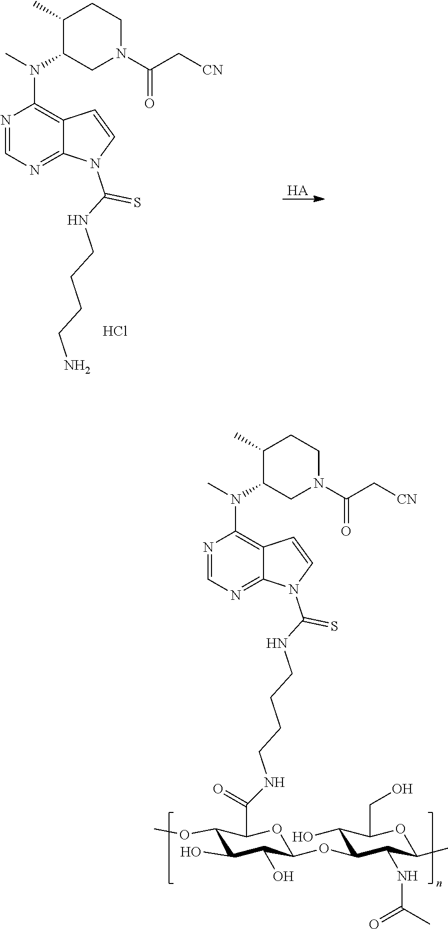

Following Step 3 of Example 1, sodium hyaluronate (MW 50 KDa, 0.170 g, 0.42 mmol, 1 eq) and N-(4-aminobutyl)-4-(((3R,4R)-1-(2-cyanoacetyl)-4-methylpiperidin-3-yl) (methyl)amino)-7H-pyrrolo[2,3-d]pyrimidine-7-carbothioamide hydrochloride (0.2 g, 0.42 mmol, 1 eq) afforded the title compound (0.18 g, yield: 53%, DSR=20%); ¹H-NMR (400 MHz, D₂O) δ ppm 8.8-7.4 (m, 0.48H), 7.0-6.0 (m, 0.12H), 4.7-4.25 (m, 2H), 4.18-3.03 (m, 12.8H), 2.47 (br, 0.2H), 2.03 (s, 3H), 1.83 (br, 1H), 1.30 (br, 0.2H), 1.15-1.0 (m, 0.6H).

With Step 3 of Example 1, reaction of sodium hyaluronate (MW 500 KDa) provided corresponding product (0.196 g, yield: 56.8%, DSR=30%)

With Step 3 of Example 1, reaction of sodium hyaluronate (MW 2000 KDa) provided corresponding product (0.203 g, yield: 58.8%, DSR=8%).

### Example 23

### Preparation of conjugate of N-(4-(aminomethyl)phenyl)-4-(((3R,4R)-1-(2-cyanoacetyl)-4-methylpiperidin-3-yl)(methyl)amino)-7H-pyrrolo[2,3-d]pyrimidine-7-carbothioamide and HA

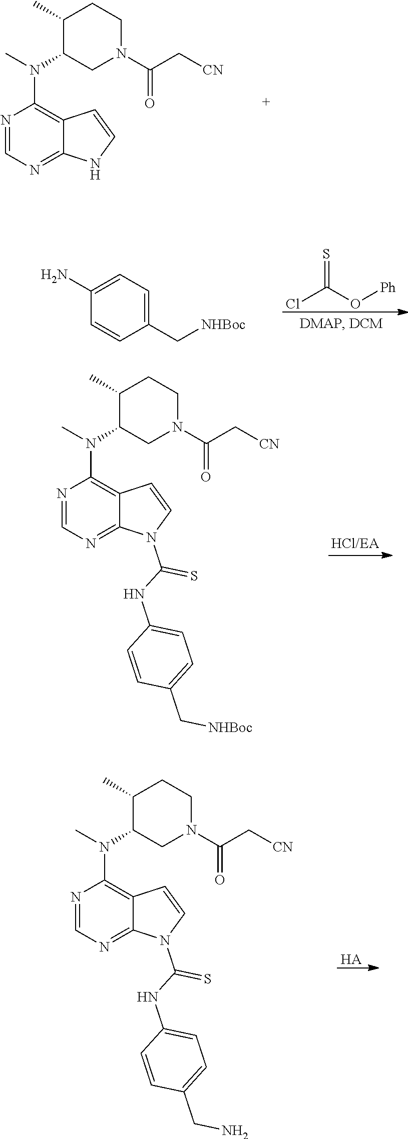

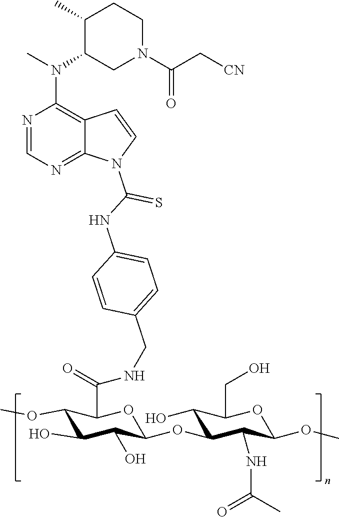

### Step 1: Preparation of tert-butyl (4-(4-(((3R,4R)-1-(2-cyanoacetyl)-4-methylpiperidin-3-yl)(methyl)amino)-7H-pyrrolo[2,3-d]pyrimidine-7-carbothioamido)benzyl)carbamate

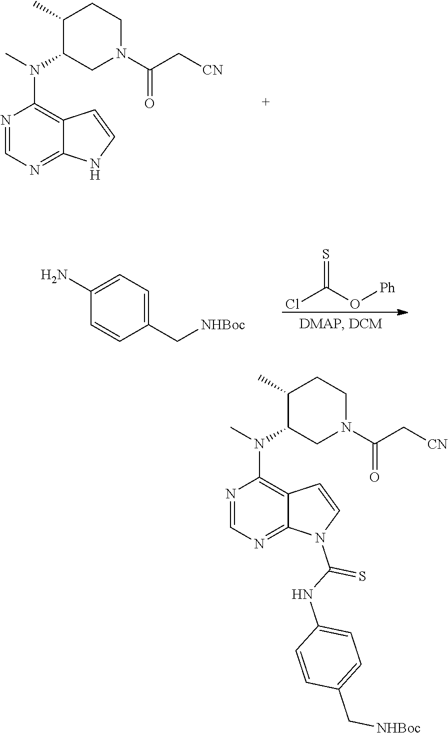 [SMILES: C[C@@H]1CCN(C(=O)CC#N)C[C@@H]1N(C)c1ncnc2[nH]ccc12 ; CC(C)(C)OC(=O)NCc1ccc(N)cc1 ; S=C(Cl)Oc1ccccc1 ; C[C@@H]1CCN(C(=O)CC#N)C[C@@H]1N(C)c1ncnc2c1ccn2C(=S)Nc1ccc(CNC(=O)OC(C)(C)C)cc1]

Following Step 1 of Example 22, tofacitinib (3 g, 9.6 mmol, 1 eq) and tert-butyl (4-aminobenzyl) carbamate (2.134 g, 9.6 mmol, 1 eq) gave the title product (0.3 g, yield: 5.5%); MS (m/z): [M+H]+ calcd for C₂₉H₃₆N₈O₃S, 577.26; found, 577.2.

### Step 2: Preparation of N-(4-(aminomethyl)phenyl)-4-(((3R,4R)-1-(2-cyanoacetyl)-4-methylpiperidin-3-yl)(methyl)amino)-7H-pyrrolo[2,3-d]pyrimidine-7-carbothioamide hydrochloride

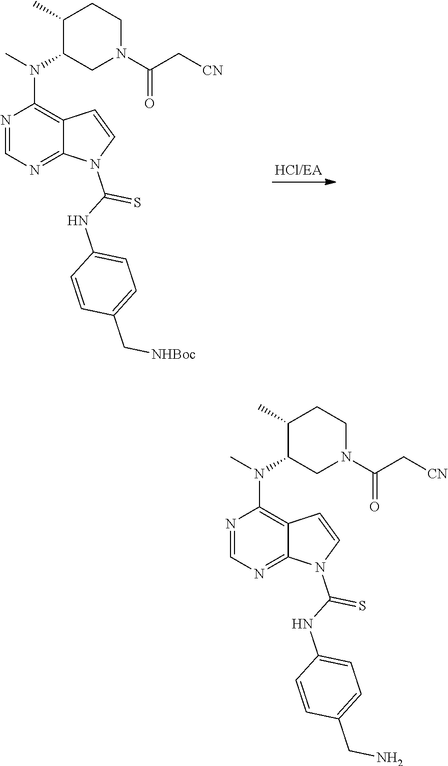 [SMILES: C[C@@H]1CCN(C(=O)CC#N)C[C@@H]1N(C)c1ncnc2c1ccn2C(=S)Nc1ccc(CNC(=O)OC(C)(C)C)cc1 ; C[C@@H]1CCN(C(=O)CC#N)C[C@@H]1N(C)c1ncnc2c1ccn2C(=S)Nc1ccc(CN)cc1]

Following Step 2 of Example 1, tert-butyl (4-(4-(((3R,4R)-1-(2-cyanoacetyl)-4-methylpiperidin-3-yl)(methyl)amino)-7H-pyrrolo[2,3-d]pyrimidine-7-carbothioamido)benzyl)carbamate (0.375 g, 0.65 mmol, 1 eq) gave the desired product as HCl salt (0.26 g, yield: 78.8%); MS (m/z): [M+H]+ calcd for C₂₄H₂₈N₈OS, 477.21; found, 477.2. ¹H-NMR (400 MHz, D₂O) δ ppm 8.43-8.30 (m, 2H), 7.85 (d, J=8.1 Hz, 2H), 7.51 (d, J=8.1 Hz, 2H), 6.94 (s, 1H), 4.1-3.65 (m, 6H), 3.63-3.25 (m, 6H), 2.47 (s, 1H), 1.93-1.56 (m, 2H), 1.15-1.05 (m, 3H).

### Step 3: Preparation of conjugate of N-(4-(aminomethyl)phenyl)-4-(((3R,4R)-1-(2-cyanoacetyl)-4-methylpiperidin-3-yl)(methyl)amino)-7H-pyrrolo[2,3-d]pyrimidine-7-carbothioamide and HA

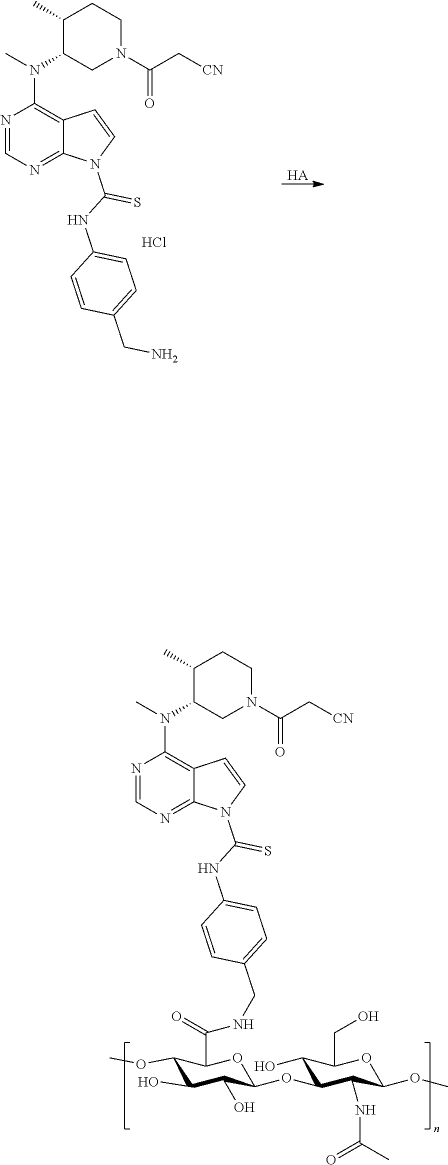

Following Step 3 of Example 1, sodium hyaluronate (MW 50 KDa, 0.157 g, 0.39 mmol, 1 eq) and N-(4-(aminomethyl)phenyl)-4-(((3R,4R)-1-(2-cyanoacetyl)-4-methylpiperidin-3-yl)(methyl)amino)-7H-pyrrolo[2,3-d] pyrimidine-7-carbothioamide hydrochloride (0.2 g, 0.39 mmol, 1 eq) afforded the title compound (0.18 g, yield: 55%, DSR=12%); ¹H-NMR (400 MHz, D₂O) δ ppm 8.5-6.75 (m, 0.91H), 4.52 (m, 3H), 4.24-2.85 (m, 10.45H), 2.55-2.42 (m, 0.12H), 2.03 (s, 3H), 1.30 (m, 0.24H), 1.13 (m, 0.36H).

With Step 3 of Example 1, reaction of sodium hyaluronate (MW 500 KDa) provided corresponding product (0.166 g, yield: 49.8%, DSR=24.3%).

With Step 3 of Example 1, reaction of sodium hyaluronate (MW 2000 KDa) provided corresponding product (0.132 g, yield: 39.7%, DSR=14.7%).

### Example 24

### Preparation of conjugate of N-(4-(2-aminoethyl)phenyl)-4-(((3R,4R)-1-(2-cyanoacetyl)-4-methylpiperidin-3-yl)(methyl)amino)-7H-pyrrolo[2,3-d]pyrimidine-7-carbothioamide and HA

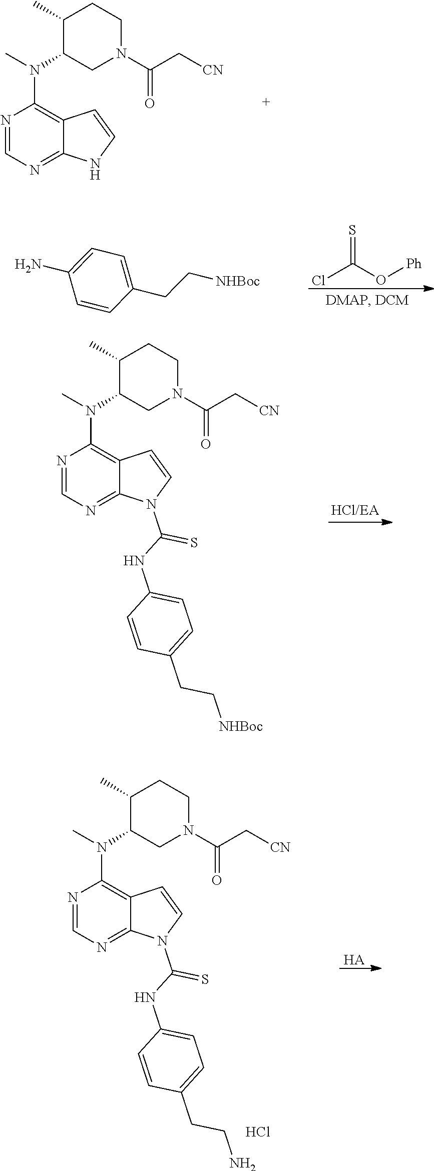

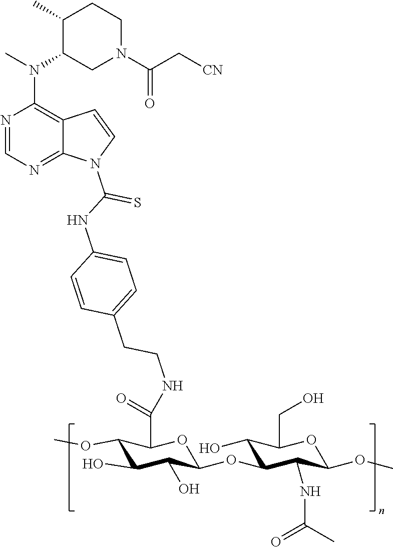

### Step 1: Preparation of tert-butyl (4-(4-(((3R,4R)-1-(2-cyanoacetyl)-4-methylpiperidin-3-yl)(methyl)amino)-7H-pyrrolo[2,3-d]pyrimidine-7-carbothioamido)phenethyl)carbamate

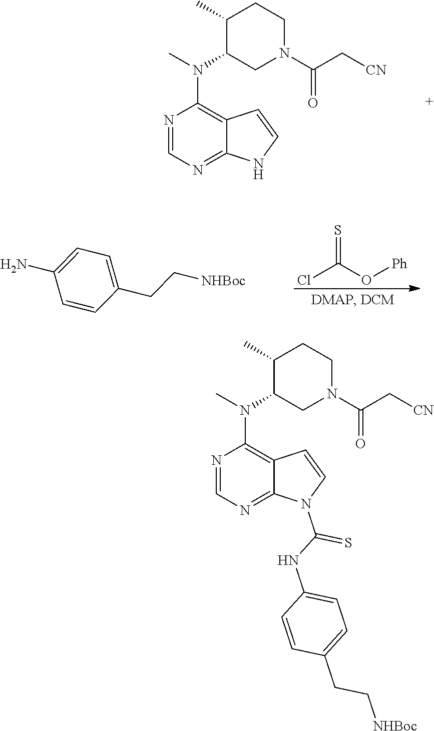 [SMILES: C[C@@H]1CCN(C(=O)CC#N)C[C@@H]1N(C)c1ncnc2[nH]ccc12 ; CC(C)(C)OC(=O)NCCc1ccc(N)cc1 ; S=C(Cl)Oc1ccccc1 ; C[C@@H]1CCN(C(=O)CC#N)C[C@@H]1N(C)c1ncnc2c1ccn2C(=S)Nc1ccc(CCNC(=O)OC(C)(C)C)cc1]

Following Step 1 of Example 22, tofacitinib (2 g, 6.4 mmol, 1 eq) and tert-butyl (4-aminophenethyl)carbamate (1.512 g, 6.4 mmol, 1 eq) gave the title product (0.78 g, yield: 20.6%); MS (m/z): [M+H]+ calcd for C₃₀H₃₈N₈O₃S, 591.28; found, 591.2.

### Step 2: Preparation of N-(4-(2-aminoethyl)phenyl)-4-(((3R,4R)-1-(2-cyano acetyl)-4-methylpiperidin-3-yl)(methyl)amino)-7H-pyrrolo[2,3-d]pyrimidine-7-carbothioamide hydrochloride

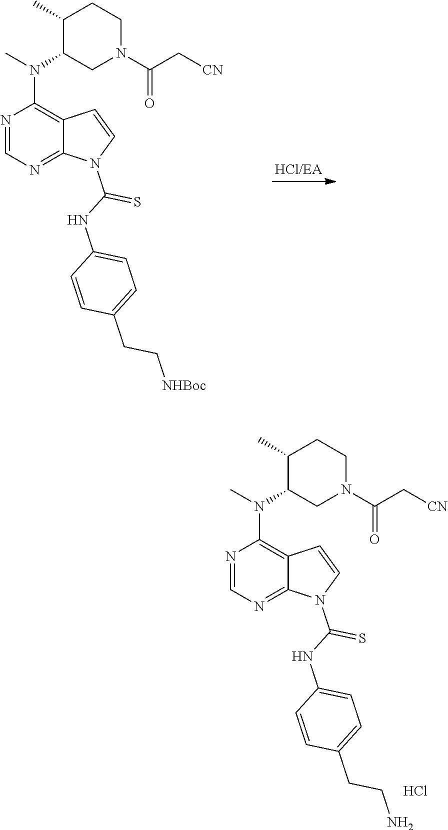 [SMILES: C[C@@H]1CCN(C(=O)CC#N)C[C@@H]1N(C)c1ncnc2c1ccn2C(=S)Nc1ccc(CCNC(=O)OC(C)(C)C)cc1 ; C[C@@H]1CCN(C(=O)CC#N)C[C@@H]1N(C)c1ncnc2c1ccn2C(=S)Nc1ccc(CCN)cc1 ; Cl]

Following Step 2 of Example 1, tert-butyl (4-(4-(((3R,4R)-1-(2-cyanoacetyl)-4-methylpiperidin-3-yl)(methyl)amino)-7H-pyrrolo[2,3-d]pyrimidine-7-carbothioamido)phenethyl) carbamate (0.55 g, 0.931 mmol, 1 eq) gave the desired product as HCl salt (0.49 g, yield: 100%); MS (m/z): [M+H]+ calcd for C₂₅H₃₀N₈₀S, 491.23; found, 491.1. ¹H-NMR (400 MHz, D₂O) δ ppm 8.43-8.30 (m, 2H), 7.73 (d, J=7.7 Hz, 2H), 7.34 (d, J=7.9 Hz, 2H), 6.95 (s, 1H), 4.69 (s, 1H), 3.97-3.71 (m, 3H), 3.61-3.37 (m, 4H), 3.23-3.04 (m, 4H), 2.95 (t, 2H), 2.48 (s, 1H), 1.92-1.62 (m, 2H), 1.12 (dd, J=13.9, 6.0 Hz, 3H).

### Step 3: Preparation of conjugate of N-(4-(2-aminoethyl)phenyl)-4-(((3R,4R)-1-(2-cyanoacetyl)-4-methylpiperidin-3-yl)(methyl)amino)-7H-pyrrolo[2,3-d]pyrimidine-7-carbothioamide and HA

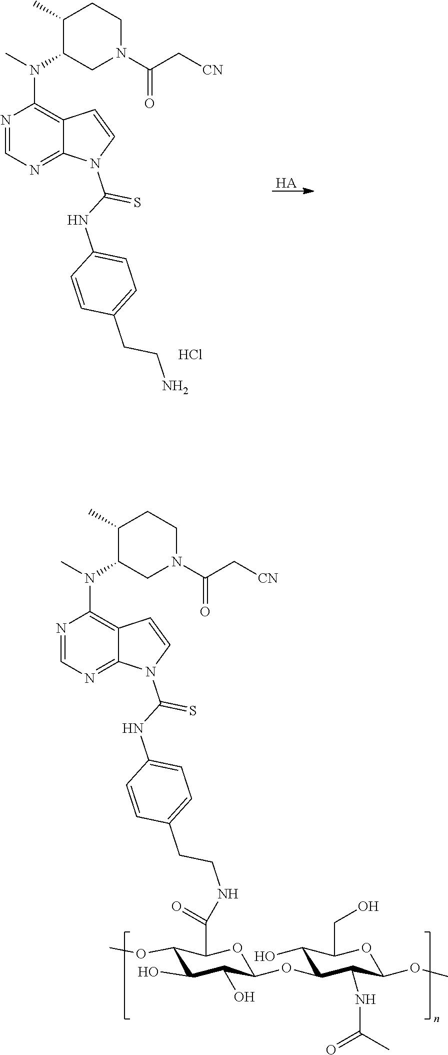

Following Step 3 of Example 1, sodium hyaluronate (MW 50 KDa, 0.153 g, 0.38 mmol, 1 eq) and N-(4-(2-aminoethyl)phenyl)-4-(((3R,4R)-1-(2-cyanoacetyl)-4-methylpiperidin-3-yl)(methyl)amino)-7H-pyrrolo[2,3-d] pyrimidine-7-carbothioamide hydrochloride (0.2 g, 0.38 mmol, leg) afforded the title compound (0.18 g, yield: 55%, DSR=14%); ¹H-NMR (400 MHz, D₂O) δ ppm 8.27-6.85 (m, 0.98H), 4.46 (br, 2H), 3.97-3.15 (m, 11.96H), 2.50-2.35 (m, 0.14H), 1.98 (s, 3H), 1.3-1.2 (m, 0.14H), 1.1-0.95 (m, 0.42H).

With Step 3 of Example 1, reaction of sodium hyaluronate (MW 2000 KDa) provided corresponding product (0.185 g, yield: 56%, DSR=15%).

### Example 25

### Preparation of conjugate of N-(2-(2-(2-(2-aminoethoxy)ethoxy)ethoxy)ethyl)-4-(((3R,4R)-1-(2-cyanoacetyl)-4-methylpiperidin-3-yl)(methyl)amino)-7H-pyrrolo[2,3-d]pyrimidine-7-carbothioamide and HA

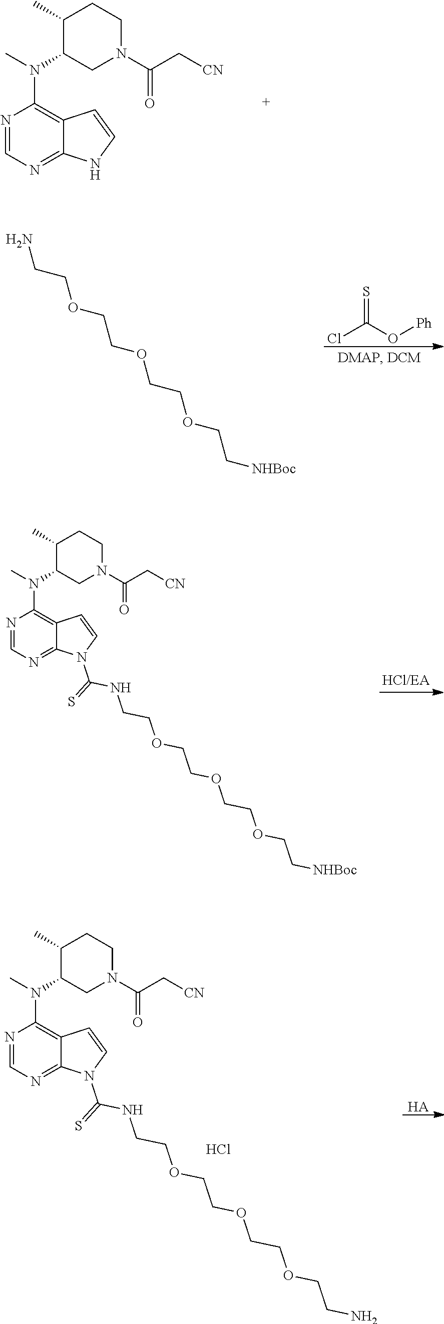

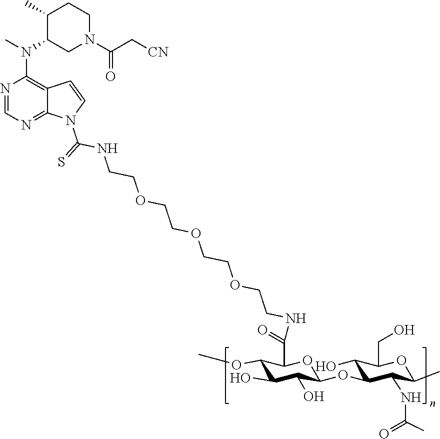

### Step 1: Preparation of tert-butyl (1-(4-(((3R,4R)-1-(2-cyanoacetyl)-4-methylpiperidin-3-yl)(methyl)amino)-7H-pyrrolo[2,3-d]pyrimidin-7-yl)-1-thioxo-5,8,11-trioxa-2-azatridecan-13-yl)carbamate

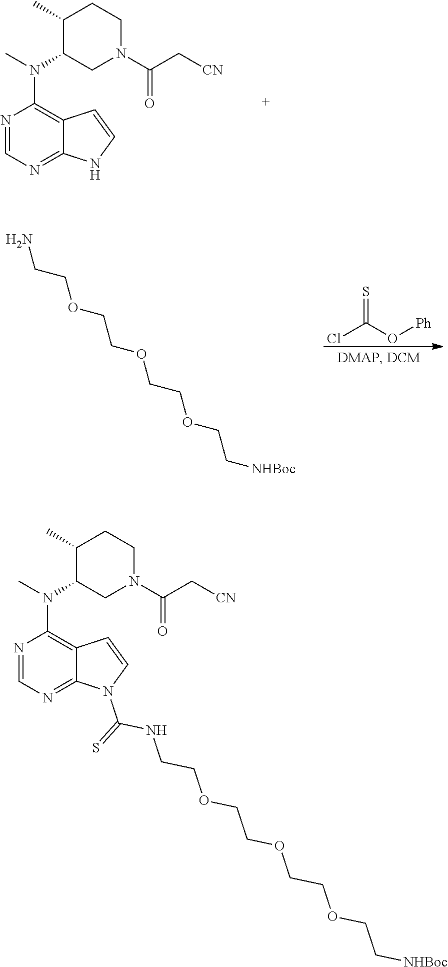 [SMILES: C[C@@H]1CCN(C(=O)CC#N)C[C@@H]1N(C)c1ncnc2[nH]ccc12 ; CC(C)(C)OC(=O)NCCOCCOCCOCCN ; S=C(Cl)Oc1ccccc1 ; C[C@@H]1CCN(C(=O)CC#N)C[C@@H]1N(C)c1ncnc2c1ccn2C(=S)NCCOCCOCCOCCNC(=O)OC(C)(C)C]

Following Step 1 of Example 22, tofacitinib (2 g, 6.4 mmol, 1 eq) and tert-butyl (2-(2-(2-(2-aminoethoxy)ethoxy)ethoxy)ethyl)carbamate (1.871 g, 6.4 mmol, 1 eq) gave the title product (1.12 g, yield: 27%); MS (m/z): [M+H]+ calcd for C₃₀H₄₆N₈O₆S, 647.33; found, 647.2.

### Step 2: Preparation of N-(2-(2-(2-(2-aminoethoxy)ethoxy)ethoxy)ethyl)-4-(((3R,4R)-1-(2-cyanoacetyl)-4-methylpiperidin-3-yl)(methyl)amino)-7H-pyrrolo[2,3-d]pyrimidine-7-carbothioamide hydrochloride

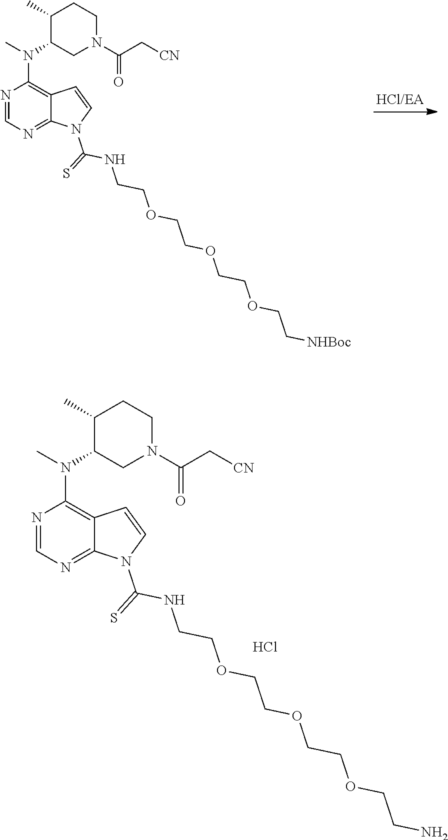 [SMILES: C[C@@H]1CCN(C(=O)CC#N)C[C@@H]1N(C)c1ncnc2c1ccn2C(=S)NCCOCCOCCOCCNC(=O)OC(C)(C)C ; C[C@@H]1CCN(C(=O)CC#N)C[C@@H]1N(C)c1ncnc2c1ccn2C(=S)NCCOCCOCCOCCN ; Cl]

Following Step 2 of Example 1, tert-butyl (1-(4-(((3R,4R)-1-(2-cyanoacetyl)-4-methylpiperidin-3-yl)(methyl)amino)-7H-pyrrolo[2,3-d]pyrimidin-7-yl)-1-thioxo-5,8,11-trioxa-2-azatridecan-13-yl)carbamate (1.1 g, 1.7 mmol, 1 eq) gave the desired product as HCl salt (0.84 g, yield: 85%); MS (m/z): [M+H]+ calcd for C₂₅H₃₈N₈O₄S, 547.27; found, 547.2. ¹H-NMR (400 MHz, D₂O) δ ppm 8.5-8.0 (m, 2H), 6.61 (s, 1H), 4.75-4.40 (m, 2H), 4.05-3.5 (m, 18H), 3.40-3.3 (m, 6H), 2.45 (br, 1H), 2.00-1.6 (m, 2H), 1.17-1.04 (m, 3H).

### Step 3: Preparation of conjugate of N-(2-(2-(2-(2-aminoethoxy)ethoxy)ethoxy)ethyl)-4-(((3R,4R)-1-(2-cyanoacetyl)-4-methylpiperidin-3-yl)(methyl)amino)-7H-pyrrolo[2,3-d]pyrimidine-7-carbothioamide and HA

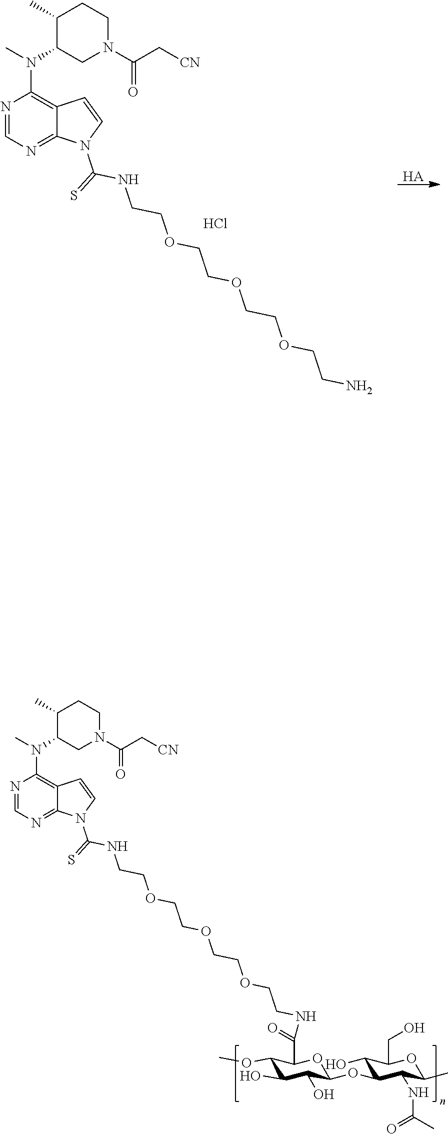

Following Step 3 of Example 1, sodium hyaluronate (MW 50 KDa, 0.137 g, 0.34 mmol, leg) and N-(2-(2-(2-(2-aminoethoxy)ethoxy)ethoxy)ethyl)-4-(((3R,4R)-1-(2-cyano acetyl)-4-methylpiperidin-3-yl)(methyl)amino)-7H-pyrrolo[2,3-d]pyrimidine-7-carbothioamide hydrochloride (0.2 g, 0.34 mmol, 1 eq) afforded the title compound (0.17 g, yield: 55%, DSR=31%); ¹H-NMR (400 MHz, D₂O) δ ppm 8.2-7.6 (m, 0.43H), 7.4-6.8 (m, 0.21H), 6.80-6.0 (m, 0.3H), 4.65-4.1 (m, 4.96H), 4.0-3.0 (m, 15H), 3.25 (m, 3.09H), 2.30 (br, 0.31H), 1.93 (s, 3H), 1.62-1.15 (m, 0.62H), 1.10-0.75 (m, 0.93H). With Step 3 of Example 1, reaction of sodium hyaluronate (MW 500 KDa) provided corresponding product (0.185 g, yield: 58.8%, DSR=25%).

With Step 3 of Example 1, reaction of sodium hyaluronate (MW 2000 KDa) provided corresponding product (0.231 g, yield: 73.4%, DSR=30%).

### Example 26

### Preparation of conjugate of methyl (4-(((3R,4R)-1-(2-cyanoacetyl)-4-methylpiperidin-3-yl)(methyl)amino)-7H-pyrrolo[2,3-d]pyrimidine-7-carbonothioyl)-L-lysinate and HA

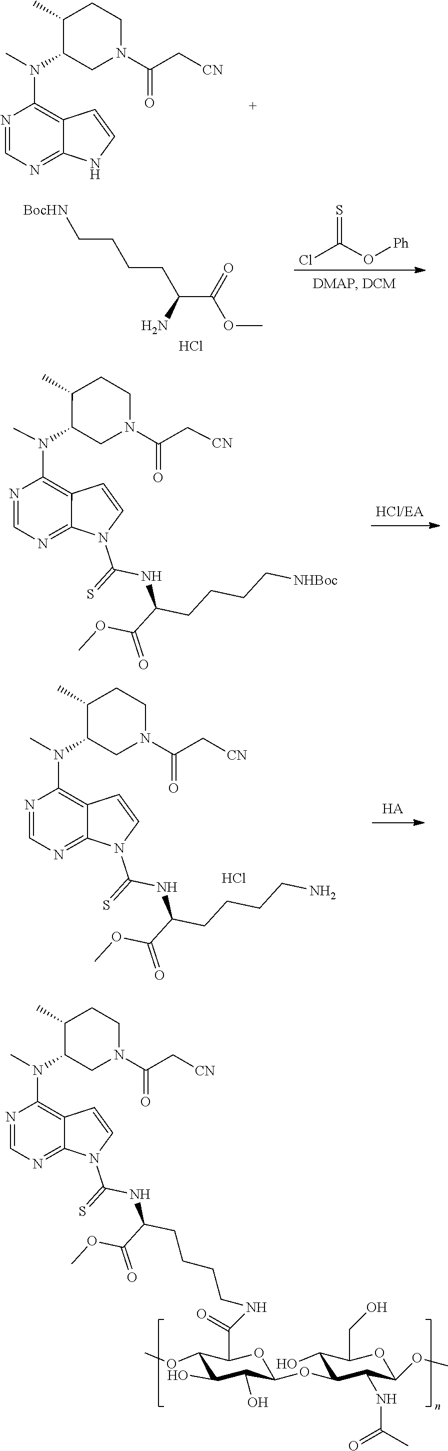

### Step 1: Preparation of methyl N6-(tert-butoxycarbonyl)-N2-(4-(((3R,4R)-1-(2-cyanoacetyl)-4-methylpiperidin-3-yl)(methyl)amino)-7H-pyrrolo[2,3-d]pyrimidine-7-carbonothioyl)-L-lysinate

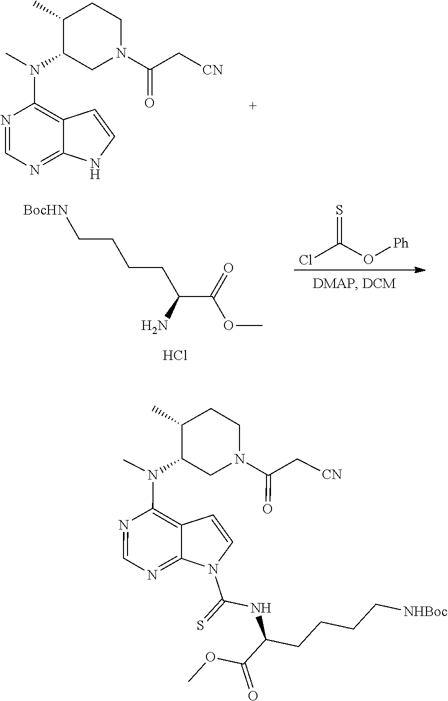 [SMILES: C[C@@H]1CCN(C(=O)CC#N)C[C@@H]1N(C)c1ncnc2[nH]ccc12 ; COC(=O)[C@@H](N)CCCCNC(=O)OC(C)(C)C ; S=C(Cl)Oc1ccccc1 ; Cl ; COC(=O)[C@H](CCCCNC(=O)OC(C)(C)C)NC(=S)n1ccc2c(N(C)[C@H]3CN(C(=O)CC#N)CC[C@H]3C)ncnc21]

Following Step 1 of Example 22, tofacitinib (2 g, 6.4 mmol, 1 eq) and methyl N6-(tert-butoxycarbonyl)-L-lysinate hydro chloride (1.9 g, 6.4 mmol, 1 eq) gave the title product (0.675 g, yield: 17.2%); MS (m/z): [M+H]+ calcd for C₂₉H₄₂N₈O₅S, 615.30; found, 615.2.

### Step 2: Preparation of methyl (4-(((3R,4R)-1-(2-cyanoacetyl)-4-methylpiperidin-3-yl)(methyl)amino)-7H-pyrrolo[2,3-d]pyrimidine-7-carbonothioyl)-L-lysinate hydrochloride

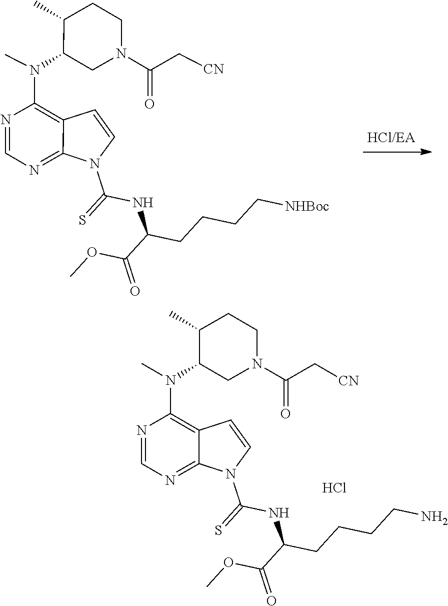

Following Step 2 of Example 1, methyl N6-(tert-butoxycarbonyl)-N2-(4-(((3R,4R)-1-(2-cyanoacetyl)-4-methylpiperidin-3-yl)(methyl)amino)-7H-pyrrolo[2,3-d]pyrimidine-7-carbonothioyl)-L-lysinate (0.625 g, 1.017 mmol, 1 eq) gave the desired product as HCl salt (0.56 g, yield: 100%); MS (m/z): [M+H]+ calcd for C₂₄H₃₄N₈O₃S, 515.25; found, 515.2. ¹H-NMR (400 MHz, D₂O) δ ppm 8.45 (s, 1H), 8.33 (s, 1H), 6.92 (s, 1H), 5.09 (t, J=6.2 Hz, 1H), 4.63 (br, 1H), 3.95-3.67 (m, 5H), 3.60-3.28 (m, 4H), 3.23 (s, 3H), 2.88 (br, 2H), 2.47 (s, 1H), 2.21-1.96 (m, 2H), 1.92-1.42 (m, 6H), 1.10 (d, J=6.8 Hz 3H).

### Step 3: Preparation of conjugate of methyl (4-(((3R,4R)-1-(2-cyanoacetyl)-4-methyl piperidin-3-yl)(methyl)amino)-7H-pyrrolo[2,3-d]pyrimidine-7-carbonothioyl)-L-lysinate and HA

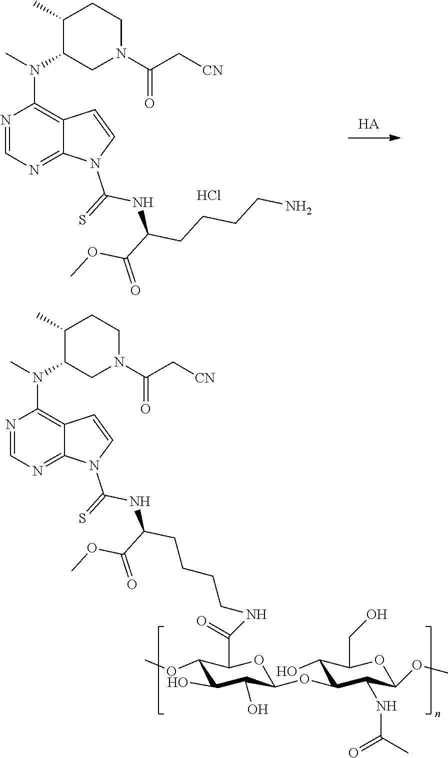

Following Step 3 of Example 1, sodium hyaluronate (MW 50 KDa, 0.145 g, 0.36 mmol, 1 eq) and methyl(4-(((3R,4R)-1-(2-cyanoacetyl)-4-methylpiperidin-3-yl)(methyl)amino)-7H-pyrrolo[2,3-d]pyrimidine-7-carbonothioyl)-L-lysinate hydrochloride (0.2 g, 0.36 mmol, 1 eq) afforded the title compound (0.13 g, yield: 41%, DSR=10%); ¹H-NMR (400 MHz, D₂O) δ ppm 8.75-7.5 (m, 0.27H), 6.87 (br, 0.031H), 5.25 (m, 0.14H), 4.65-4.21 (m, 1.86H), 4.0-2.8 (m, 11.6H), 2.44 (br, 0.1H), 2.00 (s, 3H), 1.7-1.4 (m, 0.62H), 1.28 (t, 0.1H), 1.14-1.0 (m, 0.3H).

With Step 3 of Example 1, reaction of sodium hyaluronate (MW 500 KDa) provided corresponding product (0.117 g, yield: 36.3%, DSR=10%).

With Step 3 of Example 1, reaction of sodium hyaluronate (MW 2000 KDa) provided corresponding product (0.161 g, yield: 50%, DSR=5.7%).

### Example 27

### Preparation of conjugate of 4-(aminomethyl)benzyl 4-(((3R,4R)-1-(2-cyanoacetyl)-4-methylpiperidin-3-yl)(methyl)amino)-7H-pyrrolo[2,3-d]pyrimidine-7-carboxylate and HA

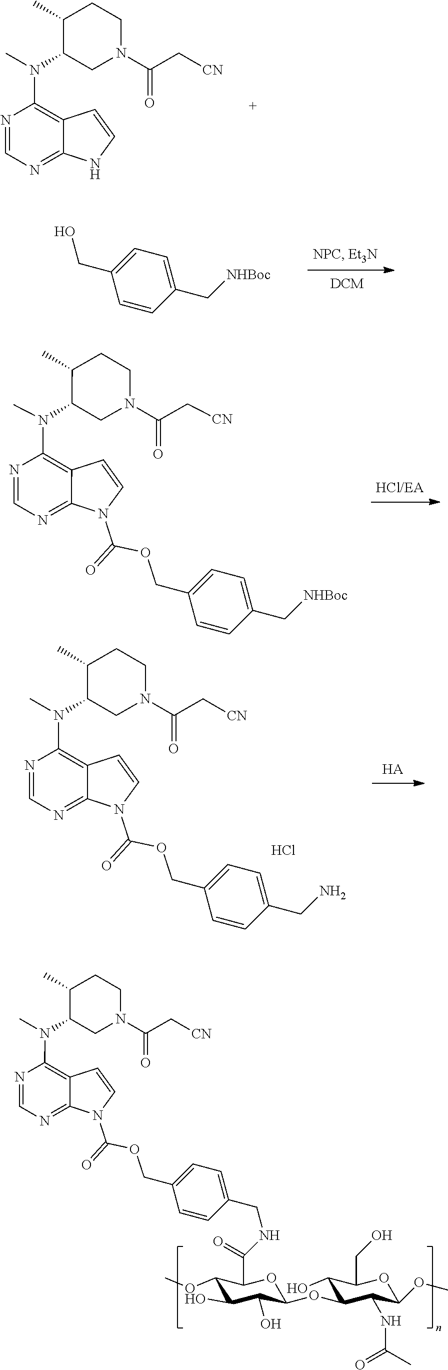

### Step 1: Preparation of 4-(((tert-butoxycarbonyl)amino)methyl)benzyl 4-(((3R,4R)-1-(2-cyanoacetyl)-4-methylpiperidin-3-yl)(methyl)amino)-7H-pyrrolo[2,3-d]pyrimidine-7-carboxylate

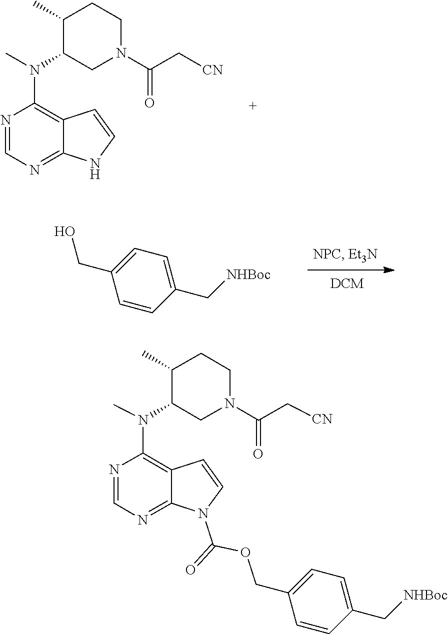

Following Step 1 of Example 1, tofacitinib (1.32 g, 4.214 mmol, 1 eq) and tert-butyl (4-(hydroxymethyl)benzyl)carbamate (2 g, 4.214 mmol, 1 eq) gave the title product (1.3 g, yield: 53.5%); MS (m/z): [M+H]+ calcd for C₃₀H₃₇N₇O₅, 576.29; found, 576.2.

### Step 2: Preparation of 4-(aminomethyl)benzyl 4-(((3R,4R)-1-(2-cyanoacetyl)-4-methylpiperidin-3-yl)(methyl)amino)-7H-pyrrolo[2,3-d]pyrimidine-7-carboxylate hydrochloride

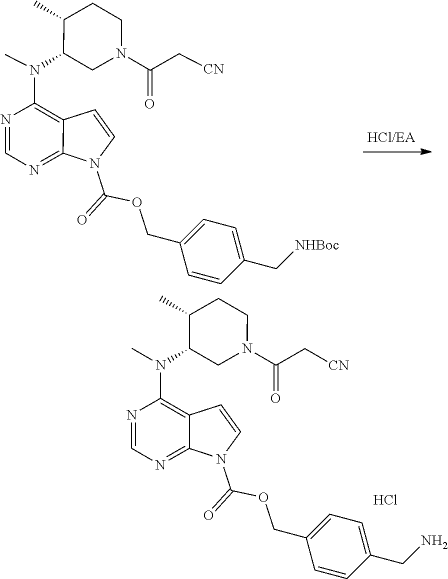

Following Step 2 of Example 1, 4-(((tert-butoxycarbonyl)amino)methyl)benzyl 4-(((3R,4R)-1-(2-cyanoacetyl)-4-methylpiperidin-3-yl)(methyl)amino)-7H-pyrrolo[2,3-d]pyrimidine-7-carboxylate (1.3 g, 2.25 mmol, 1 eq) gave the desired product as HCl salt (1 g, yield: 87%); MS (m/z): [M+H]+r calcd for C₂₅H₂₉N₇O₃, 476.23; found, 476.2. ¹H-NMR (400 MHz, D₂O) δ ppm 8.30 (dd, J=36.1, 9.5 Hz, 1H), 7.75-7.23 (m, 5H), 6.84 (d, J=15.6 Hz, 1H), 4.70 (br, 3H), 4.35-3.71 (m, 6H), 3.71-3.23 (m, 5H), 2.50 (d, J=33.4 Hz, 1H), 2.03-1.62 (m, 2H), 1.17-0.99 (m, 3H).

### Step 3: Preparation of conjugate of 4-(aminomethyl)benzyl 4-(((3R,4R)-1-(2-cyanoacetyl)-4-methylpiperidin-3-yl)(methyl)amino)-7H-pyrrolo[2,3-d]pyrimidine-7-carboxylate and HA

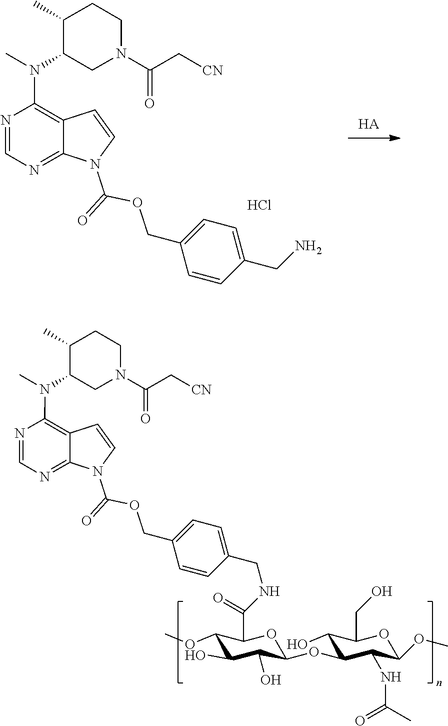

Following Step 3 of Example 1, sodium hyaluronate (MW 50 KDa, 0.157 g, 0.39 mmol, 1 eq) and 4-(aminomethyl)benzyl 4-(((3R,4R)-1-(2-cyanoacetyl)-4-methyl piperidin-3-yl)(methyl)amino)-7H-pyrrolo[2,3-d]pyrimidine-7-carboxylate hydrochloride (0.2 g, 0.39 mmol, 1 eq) afforded the title compound (0.2 g, yield: 60%, DSR=22%); ¹H-NMR (400 MHz, D₂O) δ ppm 8.37-7.89 (m, 0.18H), 7.75-7.22 (m, 1.36H), 4.71-4.32 (m, 2.75H), 4.29-3.20 (m, 12.02H), 3.14 (br, 0.34H), 2.32 (br, 0.22H), 2.02 (s, 3H), 1.31 (br, 0.45H), 1.06 (br, 0.65H).

With Step 3 of Example 1, reaction of sodium hyaluronate (MW 500 KDa) provided corresponding product (0.2 g, yield: 60%, DSR=43.6%).

With Step 3 of Example 1, reaction of sodium hyaluronate (MW 2000 KDa) provided corresponding product (0.175 g, yield: 52.6%, DSR=35.3%).

### Example 28

### Preparation of conjugate of 4-((S)-2-amino-3-methoxy-3-oxopropyl)phenyl 4-(((3R,4R)-1-(2-cyanoacetyl)-4-methylpiperidin-3-yl)(methyl)amino)-7H-pyrrolo[2,3-d]pyrimidine-7-carboxylate and HA

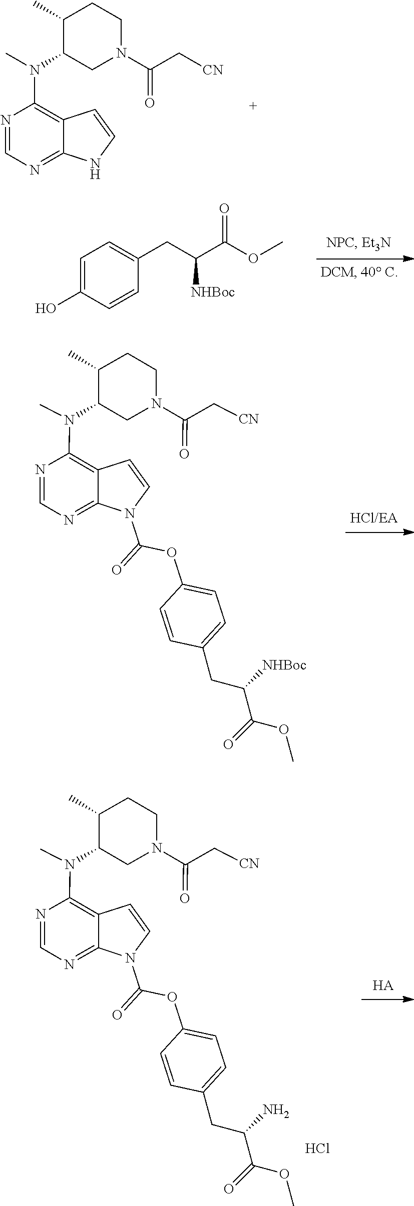

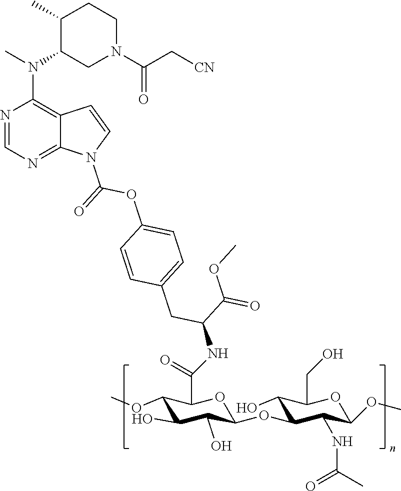

### Step 1: Preparation of 4-((S)-2-((tert-butoxycarbonyl)amino)-3-methoxy-3-oxopropyl)phenyl 4-(((3R,4R)-1-(2-cyanoacetyl)-4-methylpiperidin-3-yl)(methyl)amino)-7H-pyrrolo[2,3-d]pyrimidine-7-carboxylate

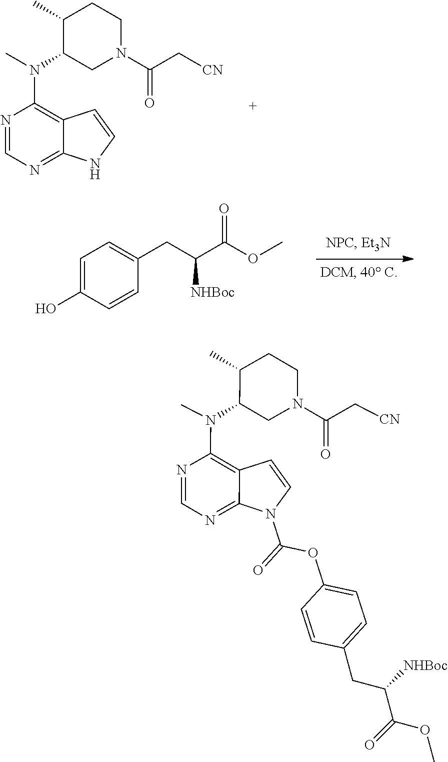

Following Step 1 of Example 1, tofacitinib (3 g, 9.62 mmol, 1 eq) and gave the title product (1.7 g, yield: 28%); MS (m/z): [M+H]+ calcd for C₃₂H₃₉N₇O₇, 634.29; found, 634.2.

### Step 2: Preparation of 4-((S)-2-amino-3-methoxy-3-oxopropyl)phenyl 4-(((3R,4R)-1-(2-cyanoacetyl)-4-methylpiperidin-3-yl)(methyl)amino)-7H-pyrrolo[2,3-d]pyrimidine-7-carboxylate hydrochloride

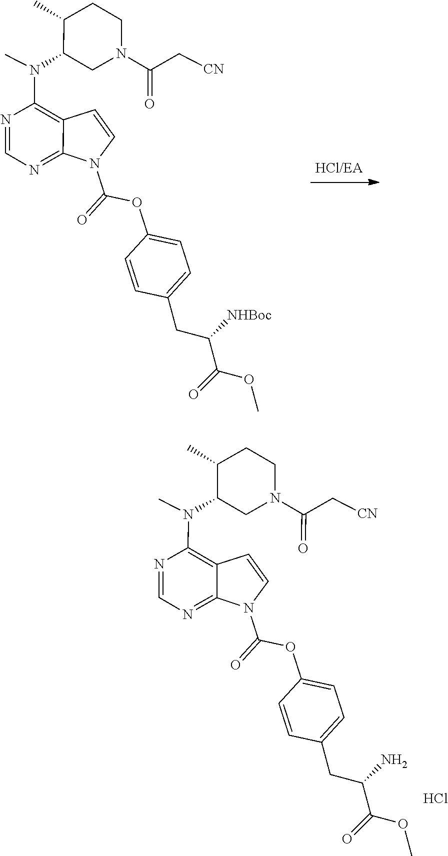

Following Step 2 of Example 1, 4-((S)-2-((tert-butoxycarbonyl)amino)-3-methoxy-3-oxopropyl)phenyl4-(((3R,4R)-1-(2-cyanoacetyl)-4-methylpiperidin-3-yl) (methyl)amino)-7H-pyrrolo[2,3-d]pyrimidine-7-carboxylate (1 g, 1.578 mmol, 1 eq) gave the desired product as HCl salt (0.9 g, yield: 100%); MS (m/z): [M+H]+ calcd for C₂₇H₃₁N₇O₅, 534.24; found, 534.2. ¹H-NMR (400 MHz, CD₃OD) δ ppm 8.55-8.34 (m, 1H), 7.52-7.42 (m, 3H), 7.41-6.74 (m, 3H), 5.28 (s, 1H), 4.46-4.21 (m, 1H), 4.09-3.85 (m, 5H), 3.82 (d, J=4.2 Hz, 3H), 3.76-3.39 (m, 5H), 3.26-3.03 (m, 1H), 2.55 (br, 1H), 2.0-1.65 (m, 2H), 1.16 (dd, J=13.2, 5.2 Hz, 3H).

### Step 3: Preparation of conjugate of 4-((S)-2-amino-3-methoxy-3-oxopropyl)phenyl 4-(((3R,4R)-1-(2-cyanoacetyl)-4-methylpiperidin-3-yl)(methyl)amino)-7H-pyrrolo[2,3-d] pyrimidine-7-carboxylate and HA

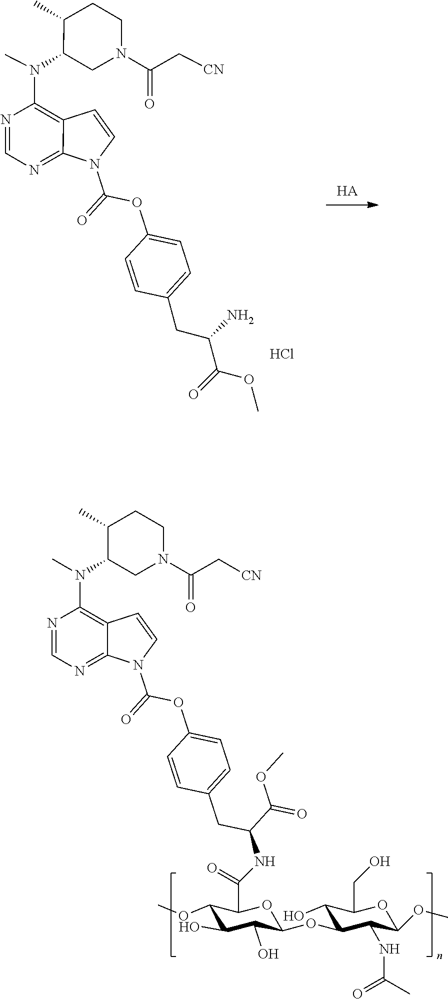

Following Step 3 of Example 1, sodium hyaluronate (MW 50 KDa, 0.141 g, 0.35 mmol, 1 eq) and 4-((S)-2-amino-3-methoxy-3-oxopropyl)phenyl 4-(((3R,4R)-1-(2-cyanoacetyl)-4-methylpiperidin-3-yl)(methyl)amino)-7H-pyrrolo[2,3-d]pyrimidine-7-carboxylate hydrochloride (0.2 g, 0.35 mmol, 1 eq) afforded the title compound (0.2 g, yield: 32%, DSR=20%); ¹H-NMR (400 MHz, D₂O) δ ppm 7.87-6.43 (m, 1.4H), 4.51 (m, 2H), 3.70 (m, 14.35H), 2.48 (m, 0.2H), 1.99 (s, 3H), 1.81-1.67 (m, 0.2H), 1.29 (m, 0.2H), 1.06 (m, 0.6H).

With Step 3 of Example 1, reaction of sodium hyaluronate (MW 500 KDa) provided corresponding product (0.16 g, yield: 50%, DSR=22%).

With Step 3 of Example 1, reaction of sodium hyaluronate (MW 2000 KDa) provided corresponding product (0.238 g, yield: 74.5%, DSR=22%).

### Example 29

### Preparation of conjugate of 8-aminooctyl 4-(((3R,4R)-1-(2-cyanoacetyl)-4-methylpiperidin-3-yl)(methyl)amino)-7H-pyrrolo[2,3-d]pyrimidine-7-carboxylate and HA

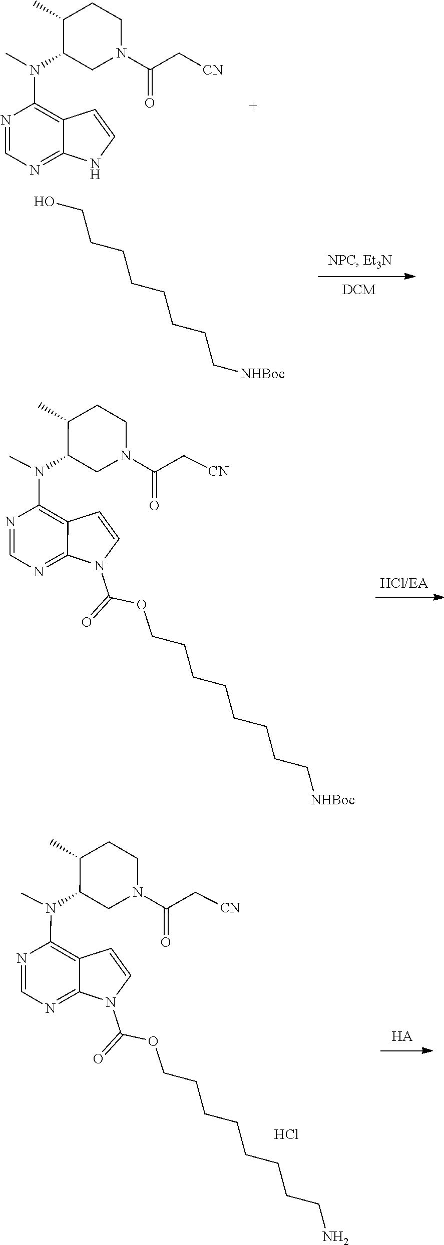

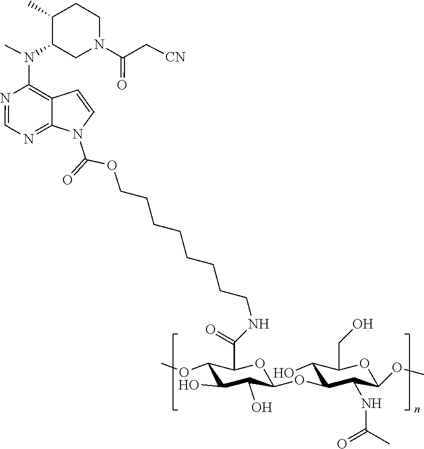

### Step 1: Preparation of 8-((tert-butoxycarbonyl)amino)octyl 4-(((3R,4R)-1-(2-cyanoacetyl)-4-methylpiperidin-3-yl)(methyl)amino)-7H-pyrrolole[2,3-d]pyrimidine-7-carboxylate

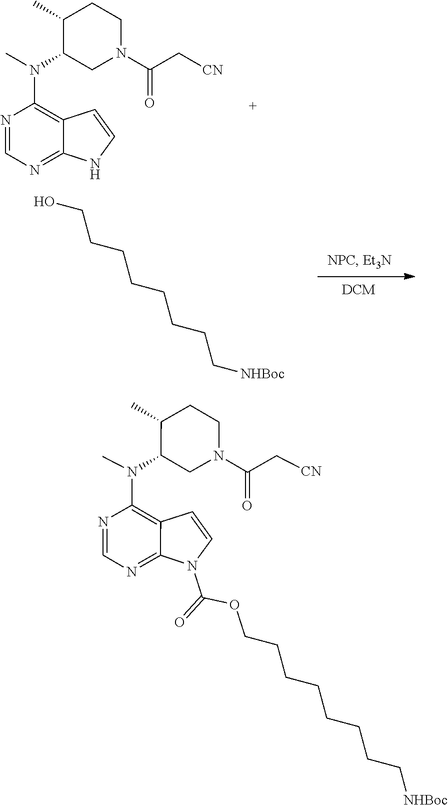

Following Step 1 of Example 1, tofacitinib (1.15 g, 3.67 mmol, 1 eq) and tert-butyl (8-hydroxyoctyl)carbamate (0.9 g, 3.668 mmol, 1 eq) gave the title product (0.7 g, yield: 33%); MS (m/z): [M+H-]+ calcd for C₃₀H₄₅N₇O₅, 584.35; found, 584.3. ¹H-NMR (400 MHz, CDCl₃) δ ppm 8.46 (d, J=8.8 Hz, 1H), 7.46 (dd, J=10.6, 4.2 Hz, 1H), 6.63 (d, J=4.1 Hz, 1H), 5.13 (s, 1H), 4.65-4.33 (m, 2H), 4.16-3.99 (m, 1H), 3.89-3.74 (m, 1H), 3.69-3.43 (m, 4H), 3.37 (d, J=18.7 Hz, 2H), 3.10 (d, J=6.4 Hz, 2H), 2.62-2.41 (m, 1H), 2.02-1.70 (m, 4H), 1.45-1.3 (m, 12H), 1.08 (dd, J=14.0, 7.1 Hz, 3H).

### Step 2: Preparation of 8-aminooctyl 4-(((3R,4R)-1-(2-cyanoacetyl)-4-methylpiperidin-3-yl)(methyl)amino)-7H-pyrrolo[2,3-d]pyrimidine-7-carboxylate hydrochloride

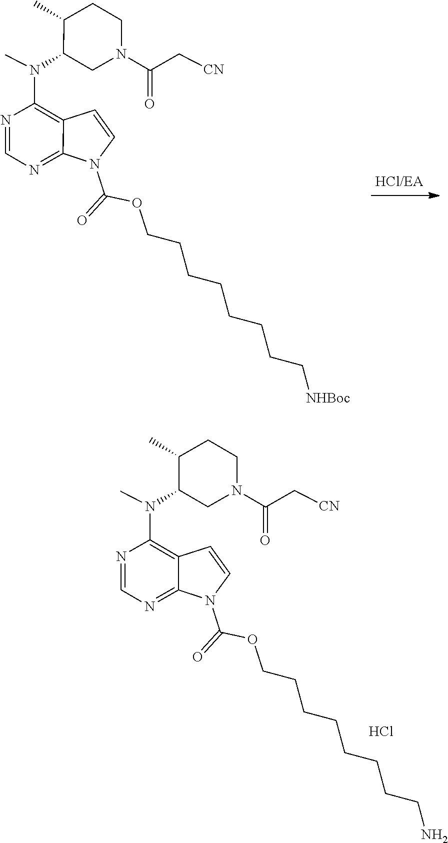 [SMILES: C[C@@H]1CCN(C(=O)CC#N)C[C@@H]1N(C)c1ncnc2c1ccn2C(=O)OCCCCCCCCNC(=O)OC(C)(C)C ; C[C@@H]1CCN(C(=O)CC#N)C[C@@H]1N(C)c1ncnc2c1ccn2C(=O)OCCCCCCCCN ; Cl]

Following Step 2 of Example 1, 8-((tert-butoxycarbonyl)amino)octyl 4-(((3R,4R)-1-(2-cyanoacetyl)-4-methylpiperidin-3-yl)(methyl)amino)-7H-pyrrolo[2,3-d]pyrimidine-7-carboxylate (0.7 g, 1.2 mmol, 1 eq) gave the desired product as HCl salt (0.6 g, yield: 96%); MS (m/z): [M+H-]+ calcd for C₂₅H₃₇N₇O₃, 484.30; found, 484.2.

### Step 3: Preparation of conjugate of 8-aminooctyl 4-(((3R,4R)-1-(2-cyanoacetyl)-4-methylpiperidin-3-yl)(methyl)amino)-7H-pyrrolo[2,3-d]pyrimidine-7-carboxylate and HA

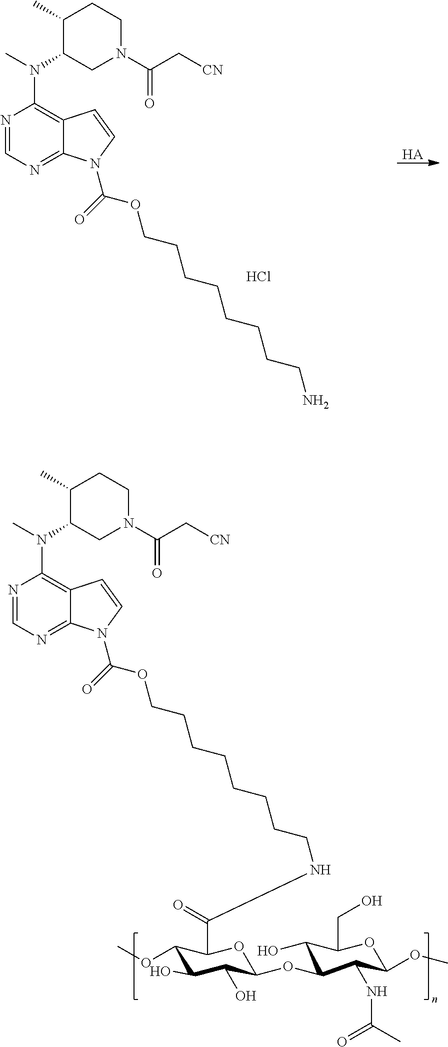

Following Step 3 of Example 1, sodium hyaluronate (MW 50 KDa, 0.153 g, 0.38 mmol, 1 eq) and 8-aminooctyl 4-(((3R,4R)-1-(2-cyanoacetyl)-4-methylpiperidin-3-yl)(methyl)amino)-7H-pyrrolo[2,3-d]pyrimidine-7-carboxylate hydrochloride (0.2 g, 0.38 mmol, 1 eq) afforded the title compound (0.19 g, yield: 54%, DSR=14%); ¹H-NMR (400 MHz, D₂O) δ ppm 8.39 (m, 0.14H), 7.67 (m, 0.14H), 6.95 (m, 0.14H), 4.53 (m, 2H), 3.73 (m, 12H), 2.51 (m, 0.24H), 2.04 (s, 3H), 1.87 (m, 0.82H), 1.48 (m, 0.92H), 1.11 (m, 0.57H).

With Step 3 of Example 1, reaction of sodium hyaluronate (MW 2000 KDa) provided corresponding product (0.191 g, yield: 58.23%, DSR=5.3%).

### Example 30

### Preparation of conjugate of 4-aminobutyl 4-[[(3R,4R)-1-(2-cyanoacetyl)-4-methyl-3-piperidyl]-methyl-amino]pyrrolo[2,3-d]pyrimidine-7-carboxylate and HA

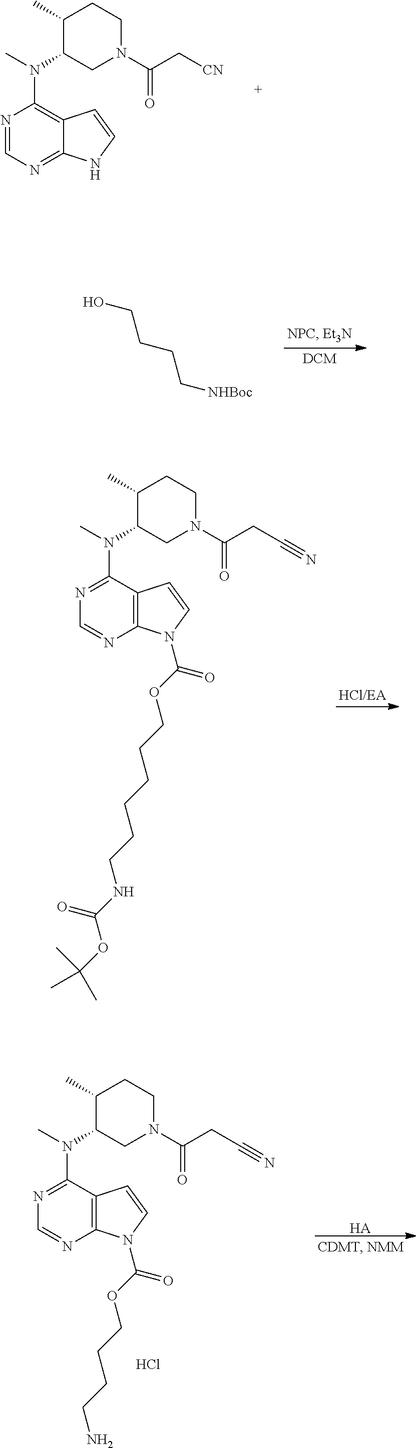 [SMILES: C[C@@H]1CCN(C(=O)CC#N)C[C@@H]1N(C)c1ncnc2[nH]ccc12 ; CC(C)(C)OC(=O)NCCCCO ; C[C@@H]1CCN(C(=O)CC#N)C[C@@H]1N(C)c1ncnc2c1ccn2C(=O)OCCCCCCNC(=O)OC(C)(C)C ; C[C@@H]1CCN(C(=O)CC#N)C[C@@H]1N(C)c1ncnc2c1ccn2C(=O)OCCCCN ; Cl ; "HA"]

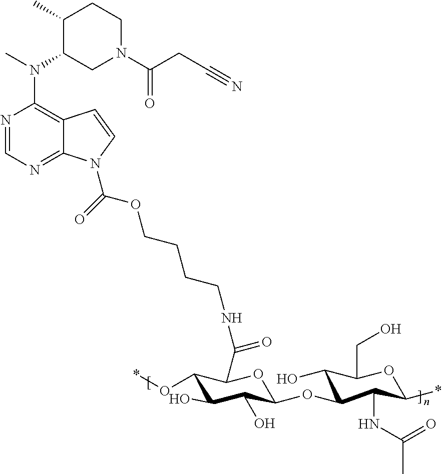

### Step 1: Preparation of 4-(tert-butoxycarbonylamino)butyl 4-[[(3S,4S)-1-(2-cyanoacetyl)-4-methyl-3-piperidyl]-methyl-amino]pyrrolo[2,3-d]pyrimidine-7-carboxylate

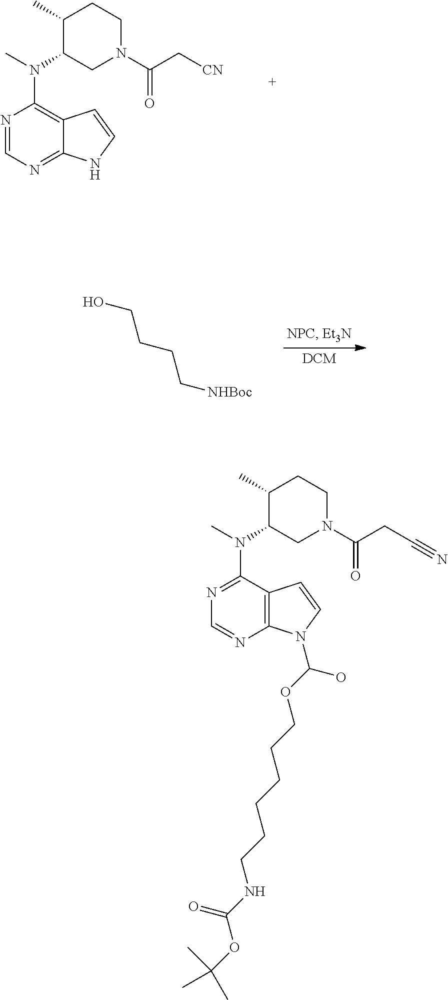 [SMILES: C[C@@H]1CCN(C(=O)CC#N)C[C@@H]1N(C)c1ncnc2[nH]ccc12 ; CC(C)(C)OC(=O)NCCCCO ; C[C@@H]1CCN(C(=O)CC#N)C[C@@H]1N(C)c1ncnc2c1ccn2C(O)OCCCCCCNC(=O)OC(C)(C)C]

To a stirred mixture of tofacitinib (3000 mg, 9.6 mmol) and bis(4-nitrophenyl) carbonate (3.22 mg, 10.6 mmol) in dichloromethane (60 mL) was added triethylamine (2.7 mL, 19.2 mmol). The reaction mixture was heated to 45° C. and stirred at this temperature for 5 h. Then the reaction mixture was cooled to room temperature. 4-(tert-Butoxycarbonylamino)-1-butanol (2000 mg, 10.6 mmol) was added. The reaction mixture was stirred at room temperature for 16 h. The solution was diluted with dichloromethane and washed with saturated NaHCO₃ solution, water and saturated brine solution. The organic layer was dried over sodium sulfate, filtered and then concentrated under reduced pressure. The crude residue was purified by column chromatography (ethyl acetate:Hexane=1:1) to afford the title compound (2.64 g, Yield: 52.1%). MS (m/z): [M+H]+ calcd for C₂₆H₃₇N₇O₅, 528.28; found, 528.2.

### Step 2: Preparation of 4-aminobutyl 4-[[(3R,4R)-1-(2-cyanoacetyl)-4-methyl-3-piperidyl]-methyl-amino]pyrrolo[2,3-d]pyrimidine-7-carboxylate hydrochloride

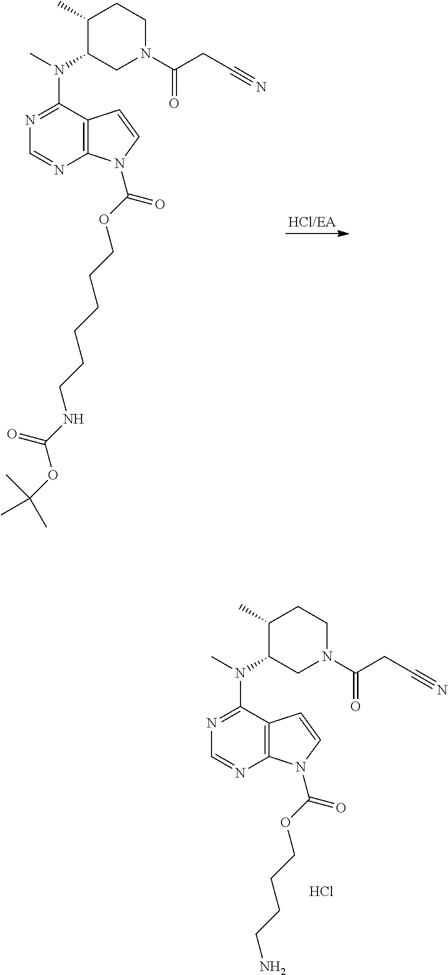 [SMILES: C[C@@H]1CCN(C(=O)CC#N)C[C@@H]1N(C)c1ncnc2c1ccn2C(=O)OCCCCCCNC(=O)OC(C)(C)C ; C[C@@H]1CCN(C(=O)CC#N)C[C@@H]1N(C)c1ncnc2c1ccn2C(=O)OCCCCN ; Cl]

Following Step 2 of Example 1, 4-(tert-butoxycarbonylamino)butyl 4-[[(3R,4R)-1-(2-cyanoacetyl)-4-methyl-3-piperidyl]-methyl-amino]pyrrolo[2,3-d]pyrimidine-7-carboxylate (2.955 g, 5.6 mmol) afford the title compound as a white solid (2.59 g, Yield: 100%) MS (m/z): [M+H]+ calcd for C₂₁H₂₉N₇O₃, 428.23; found, 428.2. ¹H NMR (400 MHz, D₂O) δ ppm 8.39 (d, J=6.9 Hz, 1H), 7.63 (d, J=3.8 Hz, 1H), 6.96 (s, 1H), 5.04 (s, 1H), 4.53 (t, J=6.2 Hz, 2H), 4.01-3.70 (m, 4H), 3.67-3.54 (m, 1H), 3.41 (d, J=30.2 Hz, 4H), 3.02 (dd, J=17.9, 10.6 Hz, 2H), 2.46 (s, 1H), 1.97-1.59 (m, 6H), 1.12-0.96 (m, 3H).

### Step 3: Preparation of conjugate of 4-aminobutyl 4-[[(3R,4R)-1-(2-cyanoacetyl)-4-methyl-3-piperidyl]-methyl-amino]pyrrolo[2,3-d]pyrimidine-7-carboxylate and HA

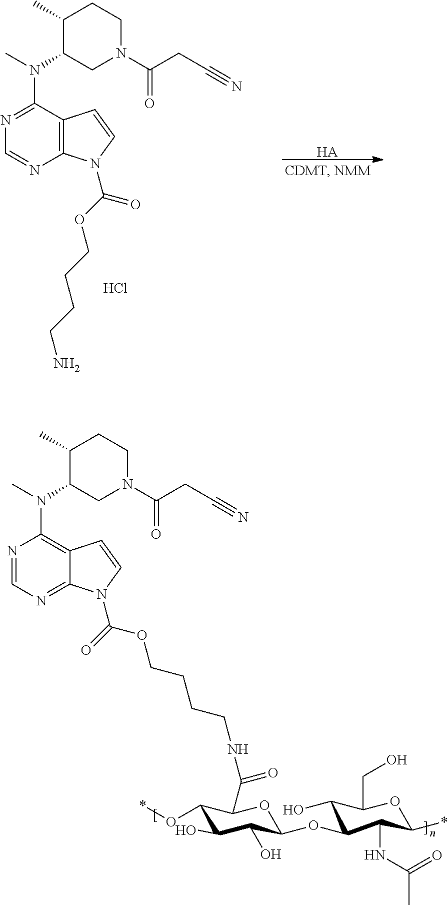

Following Step 3 of Example 1, Sodium hyaluronate (MW 50 KDa, 161.2 mg, 0.399 mmol 4-aminobutyl-4-[[(3R,4R)-1-(2-cyanoacetyl)-4-methyl-3-piperidyl]-methyl-amino]pyrrolo[2,3-d]pyrimidine-7-carboxylate hydrochloride (200 mg, 0.399 mmol) afforded the title compound 0.13 g, Yield: 36.7%, DSR=12%; ¹H NMR (400 MHz, D₂O) δ ppm 8.20-8.02 (m, 0.12H), 7.53-7.25 (m, 0.12H), 6.82-6.62 (m, 0.12H), 4.53-4.15 (m, 2.12H), 3.93-2.89 (m, 11.56H), 2.02-1.44 (m, 3.6H), 1.18-1.09 (m, 0.12H), 1.01-0.82 (m, 0.36H).

With Step 3 of Example 1, reaction of sodium hyaluronate (MW 500 KDa) provided corresponding product (0.125 g, Yield: 35.3%, DSR=37%),

With Step 3 of Example 1, reaction of sodium hyaluronate (MW 2000 KDa) provided corresponding product (0.134 g, Yield: 37.8%, DSR=32%).

### Example 31

### Preparation of conjugate of (4-aminophenyl) 4-[[(3R,4R)-1-(2-cyanoacetyl)-4-methyl-3-piperidyl]-methyl-amino]pyrrolo[2,3-d]pyrimidine-7-carboxylate and HA

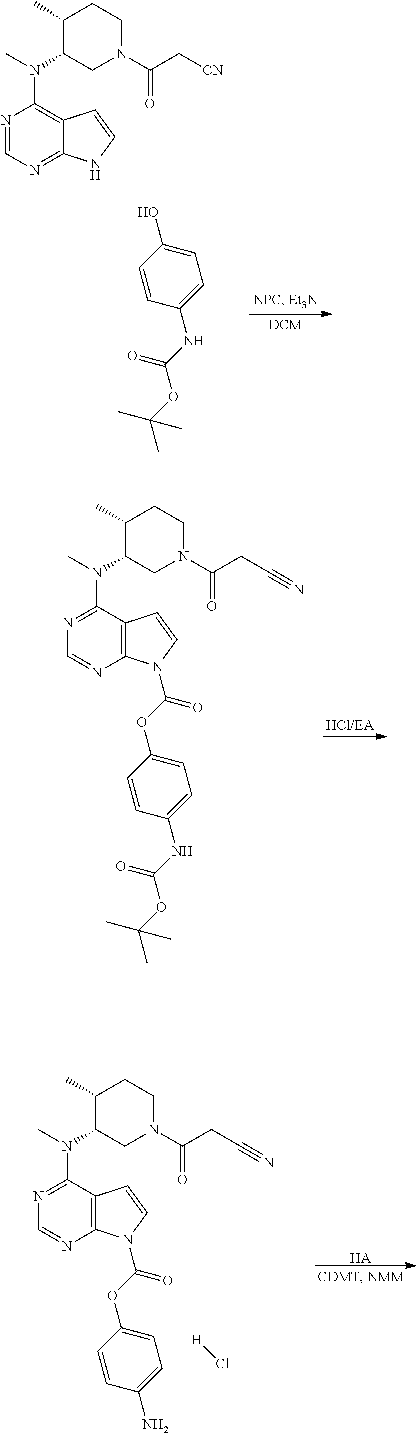

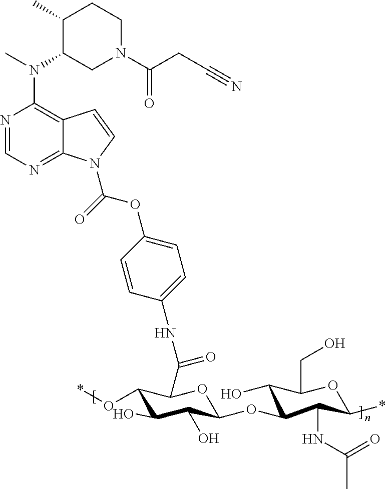

### Step 1: Preparation of [4-(tert-butoxycarbonylamino)phenyl] 4-[[(3R,4R)-1-(2-cyanoacetyl)-4-methyl-3-piperidyl]-methyl-amino]pyrrolo[2,3-d]pyrimidine-7-carboxylate

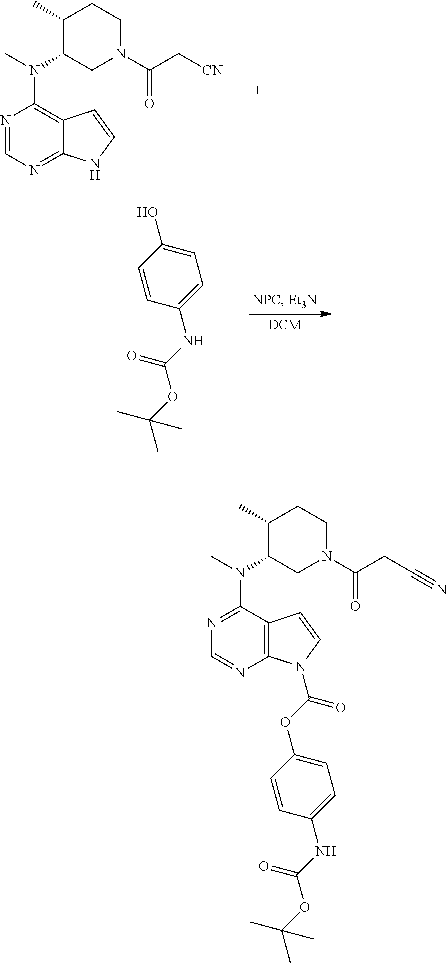 [SMILES: C[C@@H]1CCN(C(=O)CC#N)C[C@@H]1N(C)c1ncnc2[nH]ccc12 ; CC(C)(C)OC(=O)Nc1ccc(O)cc1 ; C[C@@H]1CCN(C(=O)CC#N)C[C@@H]1N(C)c1ncnc2c1ccn2C(=O)Oc1ccc(NC(=O)OC(C)(C)C)cc1]

Following Step 1 of Example 1, Tofacitinib (1562 mg, 5 mmol) and Tert-Butyl (4-hydroxyphenyl)carbamate (1256 mg, 6 mmol) afford the title compound (1.1 g, Yield: 40%). MS (m/z): [M+H]+ calcd for C₂₈H₃₃N₇O₅, 548.25; found, 548.1. ¹H NMR (400 MHz, CDCl₃) δ ppm 8.48 (d, J=8.0 Hz, 1H), 8.14-8.05 (m, 1H), 7.61 (dd, J=8.0, 4.2 Hz, 1H), 7.42 (m, 1H), 7.23 (d, J=8.9 Hz, 2H), 6.89-6.74 (m, 1H), 6.58 (s, 1H), 5.15 (d, J=3.9 Hz, 1H), 4.11-4.01 (m, 1H), 3.88-3.78 (m, 1H), 3.67-3.45 (m, 4H), 3.43-3.31 (m, 3H), 2.59-2.44 (m, 1H), 1.94 (tdd, J=23.2, 8.8, 4.5 Hz, 1H), 1.83-1.69 (m, 1H), 1.51 (m, 9H), 1.15-1.03 (m, 3H).

### Step 2: Preparation of (4-aminophenyl) 4-[[(3R,4R)-1-(2-cyanoacetyl)-4-methyl-3-piperidyl]-methyl-amino]pyrrolo[2,3-d]pyrimidine-7-carboxylate hydrochloride

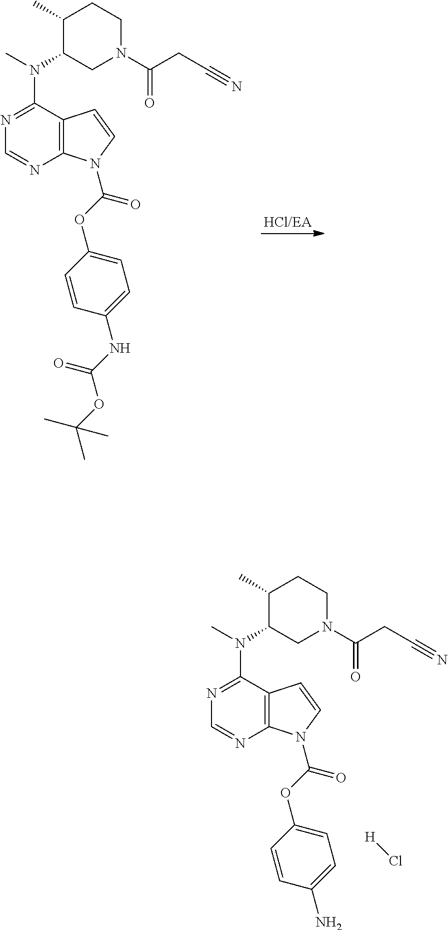

Following Step 2 of Example 1, [4-(tert-butoxycarbonylamino)phenyl] 4-[[(3R,4R)-1-(2-cyanoacetyl)-4-methyl-3-piperidyl]-methyl-amino]pyrrolo[2,3-d]pyrimidine-7-carboxylate (0.8 g, 1.46 mmol) afford the title compound as a white solid (0.52 g, Yield: 73.6%); MS (m/z): [M+H]+ calcd for C₂₃H₂₅N₇O₃, 448.20 found, 448.3.

### Step 3: Preparation of conjugate of (4-aminophenyl) 4-[[(3R,4R)-1-(2-cyanoacetyl)-4-methyl-3-piperidyl]-methyl-amino]pyrrolo[2,3-d]pyrimidine-7-carboxylate and HA

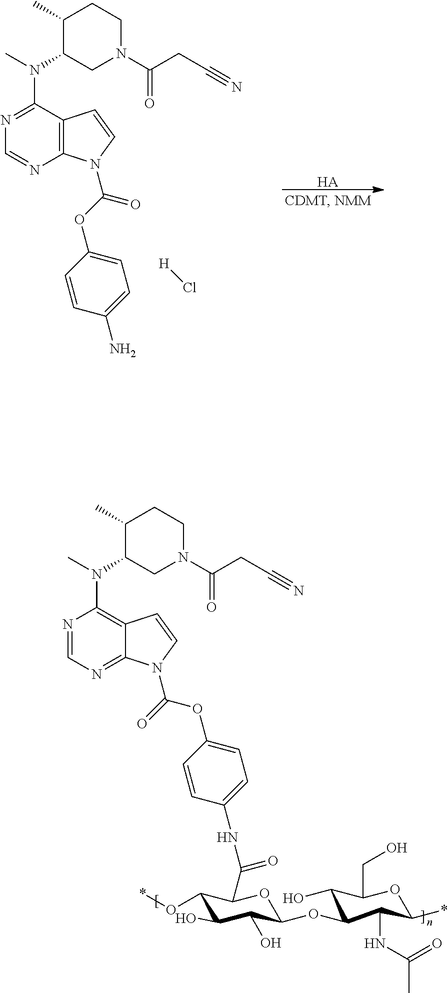

Following Step 3 of Example 1, sodium hyaluronate (MW 50 KDa, 116 mg, 0.288 mmol) and (4-aminophenyl)-4-[[(3R,4R)-1-(2-cyanoacetyl)-4-methyl-3-piperidyl]-methyl-amino]pyrrolo[2,3-d]pyrimidine-7-carboxylate hydrochloride (150 mg, 0.288 mmol) afforded the title compound 0.126 g, Yield: 48.3%, DSR: 10%; ¹H NMR (400 MHz, D₂O) δ ppm 8.26-8.17 (m, 0.1H), 7.72-7.51 (m, 0.2H), 7.38-7.11 (m, 0.3H), 6.90-6.78 (m, 0.1H), 4.53-4.25 (m, 2.1H), 4.07-3.09 (m, 10.9H), 2.49-2.34 (m, 0.1H), 2.12-1.78 (m, 3.1H), 1.61-1.50 (m, 0.1H), 1.26-0.92 (m, 0.3H).

With Step 3 of Example 1, reaction of sodium hyaluronate (MW 500 KDa) provided corresponding product (0.123 g, Yield: 47.1%, DSR: 24%),

With Step 3 of Example 1, reaction of sodium hyaluronate (MW 2000 KDa) provided corresponding product (0.138 g, Yield: 52.9%, DSR: 8%).

### Example 32

### Preparation of conjugate of azetidin-3-yl 4-[[(3R,4R)-1-(2-cyanoacetyl)-4-methyl-3-piperidyl]-methyl-amino]pyrrolo[2,3-d]pyrimidine-7-carboxylate and HA

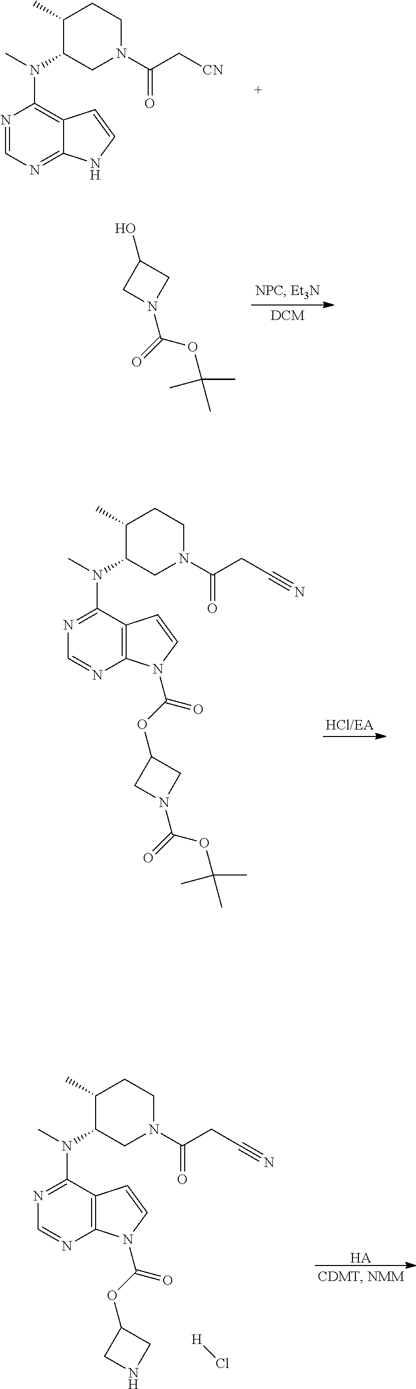 [SMILES: C[C@@H]1CCN(C(=O)CC#N)C[C@@H]1N(C)c1ncnc2[nH]ccc12 ; CC(C)(C)OC(=O)N1CC(O)C1 ; C[C@@H]1CCN(C(=O)CC#N)C[C@@H]1N(C)c1ncnc2c1ccn2C(=O)OC1CN(C(=O)OC(C)(C)C)C1 ; C[C@@H]1CCN(C(=O)CC#N)C[C@@H]1N(C)c1ncnc2c1ccn2C(=O)OC1CNC1 ; Cl ; "HA"]

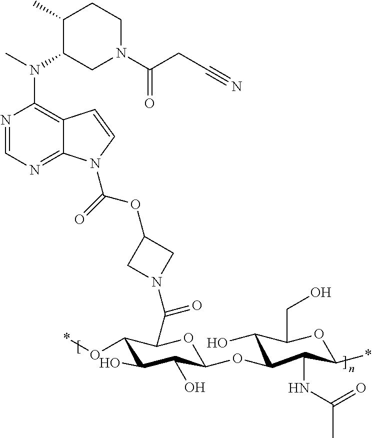

### Step 1: Preparation of (1-tert-butoxycarbonylazetidin-3-yl) 4-[[(3R,4R)-1-(2-cyanoacetyl)-4-methyl-3-piperidyl]-methyl-amino]pyrrolo[2,3-d]pyrimidine-7-carboxylate

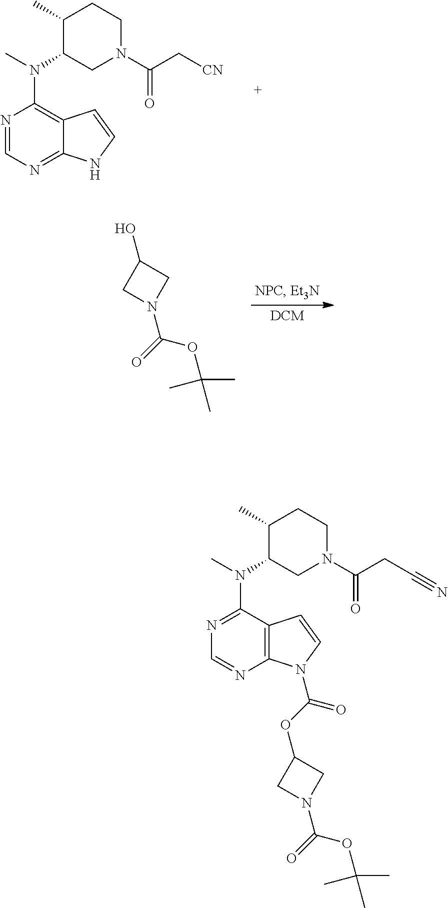 [SMILES: C[C@@H]1CCN(C(=O)CC#N)C[C@@H]1N(C)c1ncnc2[nH]ccc12 ; CC(C)(C)OC(=O)N1CC(O)C1 ; C[C@@H]1CCN(C(=O)CC#N)C[C@@H]1N(C)c1ncnc2c1ccn2C(=O)OC1CN(C(=O)OC(C)(C)C)C1]

Following Step 1 of Example 1, Tofacitinib (1249.5 mg, 4 mmol) and tert-Butyl 3-hydroxyazetidine-1-carboxylate (831.4 mg, 4.8 mmol) afford the title compound (0.48 g, Yield: 23.4%). MS (m/z): [M+H]+4 calcd for C₂₅H₃₃N₇O₅, 512.25; found, 512.1. ¹H NMR (400 MHz, CDCl₃) δ ppm 8.47 (d, J=8.4 Hz, 1H), 7.45 (dd, J=11.6, 4.2 Hz, 1H), 6.65 (dd, J=15.6, 4.0 Hz, 1H), 5.14 (d, J=3.0 Hz, 1H), 4.38 (dd, J=10.0, 7.0 Hz, 2H), 4.11-3.75 (m, 3H), 3.67-3.45 (m, 5H), 3.41-3.33 (m, 3H), 2.59-2.41 (m, 1H), 2.02-1.84 (m, 1H), 1.76 (ddd, J=23.8, 13.5, 8.5 Hz, 2H), 1.45 (d, J=6.8 Hz, 9H), 1.11-1.02 (m, 3H).

### Step 2: Preparation of azetidin-3-yl 4-[[(3R,4R)-1-(2-cyanoacetyl)-4-methyl-3-piperidyl]-methyl-amino]pyrrolo[2,3-d]pyrimidine-7-carboxylate hydrochloride

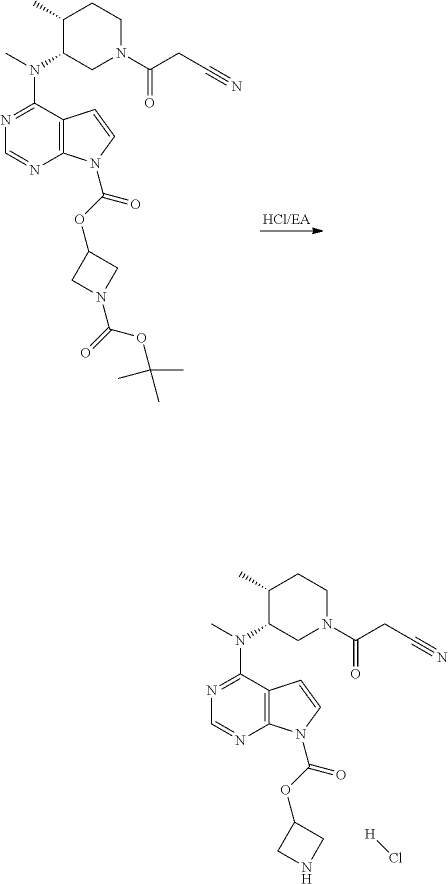 [SMILES: C[C@@H]1CCN(C(=O)CC#N)C[C@@H]1N(C)c1ncnc2c1ccn2C(=O)OC1CN(C(=O)OC(C)(C)C)C1 ; C[C@@H]1CCN(C(=O)CC#N)C[C@@H]1N(C)c1ncnc2c1ccn2C(=O)OC1CNC1 ; Cl]

Following Step 2 of Example 1, (1-tert-butoxycarbonylazetidin-3-yl) 4-[[(3R,4R)-1-(2-cyanoacetyl)-4-methyl-3-piperidyl]-methyl-amino]pyrrolo[2,3-d]pyrimidine-7-carboxylate (0.44 g, 0.86 mmol) afforded the title compound as a white solid 0.366 g, Yield: 95%). MS (m/z): [M+H]+ calcd for C₂₀H₂₅N₇O₃, 412.20; found, 412.3.

### Step 3: Preparation of conjugate of azetidin-3-yl 4-[[(3R,4R)-1-(2-cyanoacetyl)-4-methyl-3-piperidyl]-methyl-amino]pyrrolo[2,3-d]pyrimidine-7-carboxylate and HA

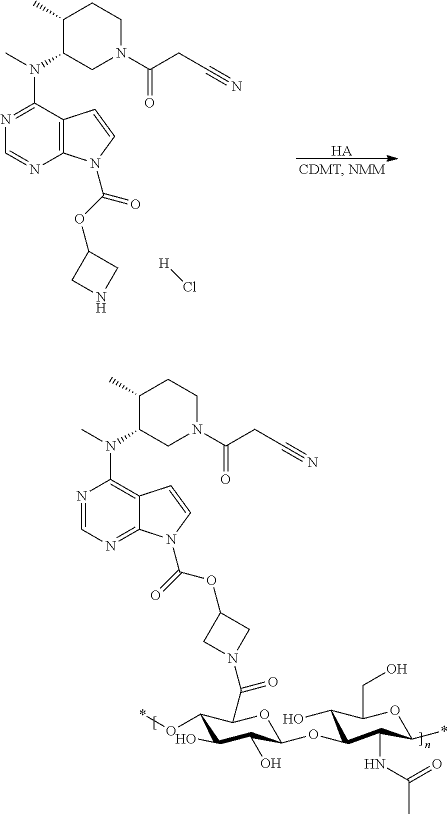

Following Step 3 of Example 1, sodium hyaluronate (MW 50 KDa, 83.2 mg, 0.207 mmol) and azetidin-3-yl4-[[(3R,4R)-1-(2-cyanoacetyl)-4-methyl-3-piperidyl]-methyl-amino]pyrrolo[2,3-d]pyrimidine-7-carboxylate hydrochloride (100 mg, 0.207 mmol) afforded the title compound 0.078 g, Yield: 43.4%, DSR: 12%; ¹H NMR (400 MHz, D₂O) δ ppm 8.27-8.17 (s, 0.12H), 7.63-7.50 (m, 0.12H), 6.92-6.78 (m, 0.12H), 4.58-4.26 (m, 7.0 Hz, 2.12H), 4.07-3.05 (m, 11.56H), 2.45-2.35 (m, 0.12H), 2.13-1.55 (m, 3.24H), 1.23 (m, 0.12H), 1.11-0.89 (m, 0.36H).

### Example 33

### Preparation of conjugate of pyrrolidin-3-yl 4-[[(3R,4R)-1-(2-cyanoacetyl)-4-methyl-3-piperidyl]-methyl-amino]pyrrolo[2,3-d]pyrimidine-7-carboxylate and HA

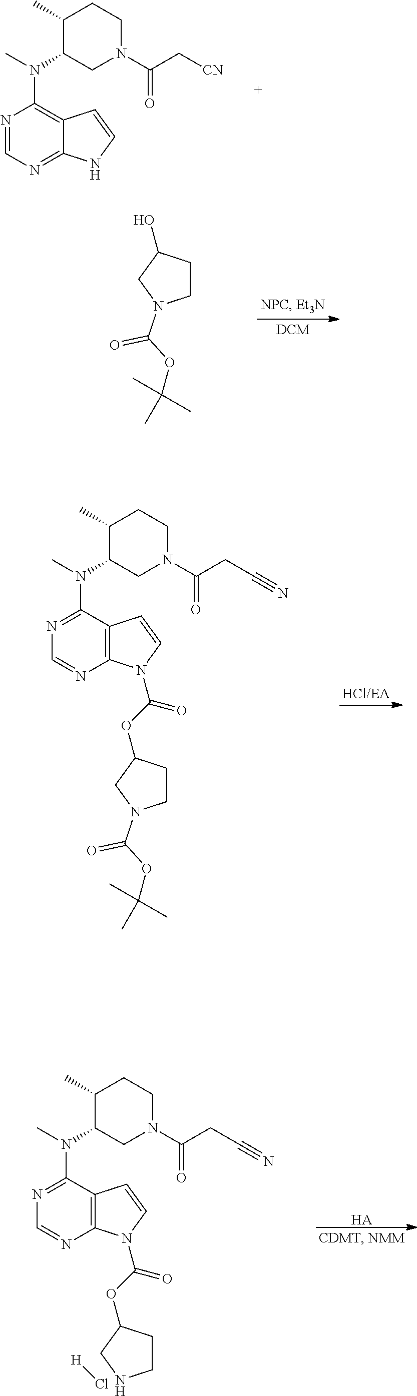

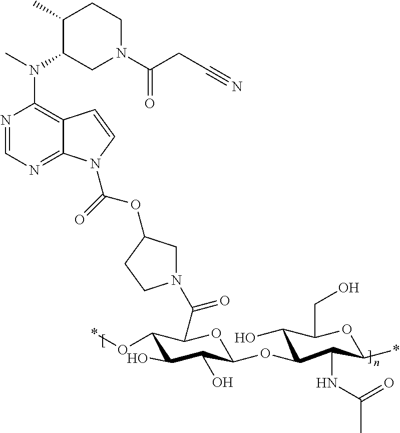

### Step 1: Preparation of (1-tert-butoxycarbonylpyrrolidin-3-yl)4-[[(3R,4R)-1-(2-cyanoacetyl)-4-methyl-3-piperidyl]-methyl-amino]pyrrolo[2,3-d]pyrimidine-7-carboxylate

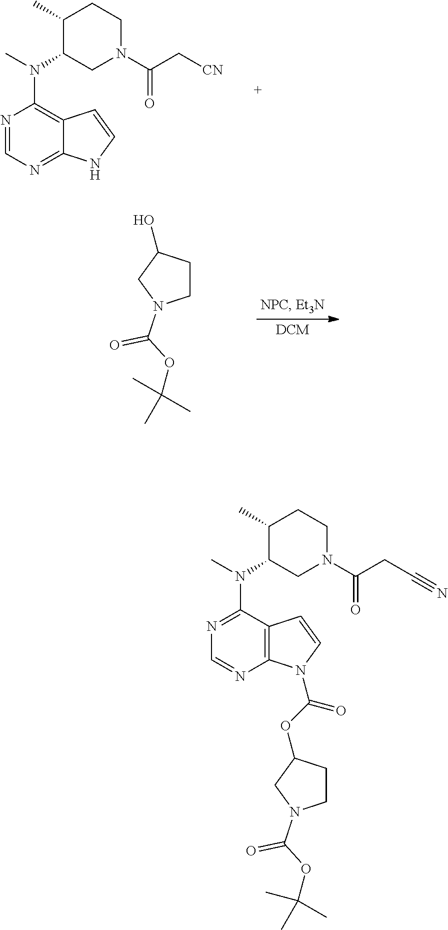 [SMILES: C[C@@H]1CCN(C(=O)CC#N)C[C@@H]1N(C)c1ncnc2[nH]ccc12 ; CC(C)(C)OC(=O)N1CCC(O)C1 ; C[C@@H]1CCN(C(=O)CC#N)C[C@@H]1N(C)c1ncnc2c1ccn2C(=O)OC1CCN(C(=O)OC(C)(C)C)C1]

Following Step 1 of Example 1, tofacitinib (1249.5 mg, 4 mmol) and tert-Butyl 3-hydroxypyrrolidine-1-carboxylate (898.8 mg, 4.8 mmol) afford the title compound (0.94 g, Yield: 44.7%). MS (m/z): [M+H]+ calcd for C₂₆H₃₅N₇O₅, 526.26; found, 526.1. ¹H NMR (400 MHz, CDCl₃) δ ppm 8.47 (d, J=6.3 Hz, 1H), 7.38 (dd, J=11.2, 4.1 Hz, 1H), 6.63 (d, J=4.1 Hz, 1H), 5.11 (t, J=11.8 Hz, 1H), 4.08-3.77 (m, 2H), 3.73-3.47 (m, 8H), 3.36 (d, J=17.7 Hz, 3H), 2.58-2.42 (m, 1H), 2.37-2.18 (m, 2H), 1.93 (dddd, J=23.5, 18.7, 9.0, 4.5 Hz, 1H), 1.82-1.72 (m, 2H), 1.47 (s, 9H), 1.08 (dd, J=14.4, 7.0 Hz, 3H).

### Step 2: Preparation of pyrrolidin-3-yl 4-[[(3R,4R)-1-(2-cyanoacetyl)-4-methyl-3-piperidyl]-methyl-amino]pyrrolo[2,3-d]pyrimidine-7-carboxylate hydrochloride

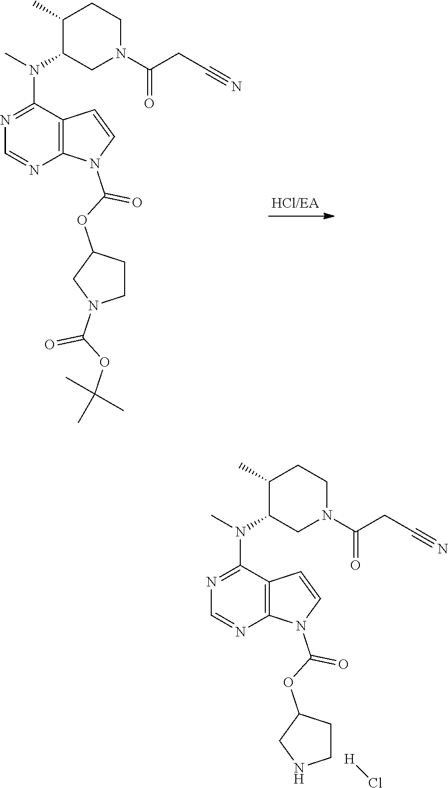

Following Step 2 of Example 1, (1-tert-butoxycarbonylpyrrolidin-3-yl) 4-[[(3R,4R)-1-(2-cyanoacetyl)-4-methyl-3-piperidyl]-methyl-amino]pyrrolo[2,3-d]pyrimidine-7-carboxylate (0.6 g, 1.14 mmol) afforded the title compound as a white solid (0.526 g, Yield: 100%). MS (m/z): [M+H]+ calcd for C₂₁H₂₇N₇O₃, 426.21; found, 426.3.

### Step 3: Preparation of conjugate of pyrrolidin-3-yl 4-[[(3R,4R)-1-(2-cyanoacetyl)-4-methyl-3-piperidyl]-methyl-amino]pyrrolo[2,3-d]pyrimidine-7-carboxylate and HA

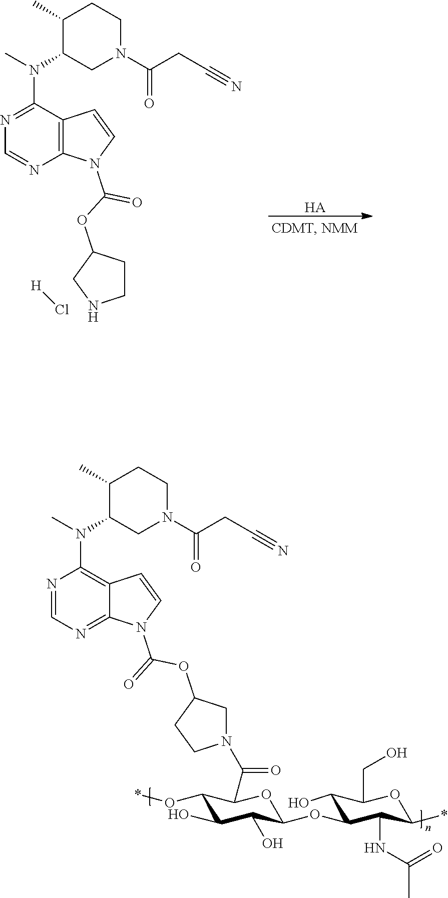

Following Step 3 of Example 1, sodium hyaluronate (MW 50 KDa, 161.6 mg, 0.401 mmol) and pyrrolidin-3-yl-4-[[(3R,4R)-1-(2-cyanoacetyl)-4-methyl-3-piperidyl]-methyl-amino]pyrrolo[2,3-d]pyrimidine-7-carboxylate hydrochloride (200 mg, 0.401 mmol) afforded the title compound 0.178 g, Yield: 50%, DSR: 20%; ¹H NMR (400 MHz, D₂O) δ ppm 8.34-8.18 (m, 0.2H), 7.66-7.38 (m, 0.2H), 6.87-6.68 (m, 0.2H), 4.58-4.26 (m, 2.2H), 4.07-3.07 (m, 12.6H), 2.53-2.19 (m, 0.6H), 2.10-1.60 (m, 3.4H), 1.29-1.18 (m, 0.2H), 1.15-0.86 (m, 0.6H).

With Step 3 of Example 1, reaction of sodium hyaluronate (MW 2000 KDa) provided corresponding product (0.185 g, Yield: 52.2%, DSR: 33%).

### Example 34

### Preparation of conjugate of 3-aminopropyl 4-[[(3R,4R)-1-(2-cyanoacetyl)-4-methyl-3-piperidyl]-methyl-amino]pyrrolo[2,3-d]pyrimidine-7-carboxylate and HA

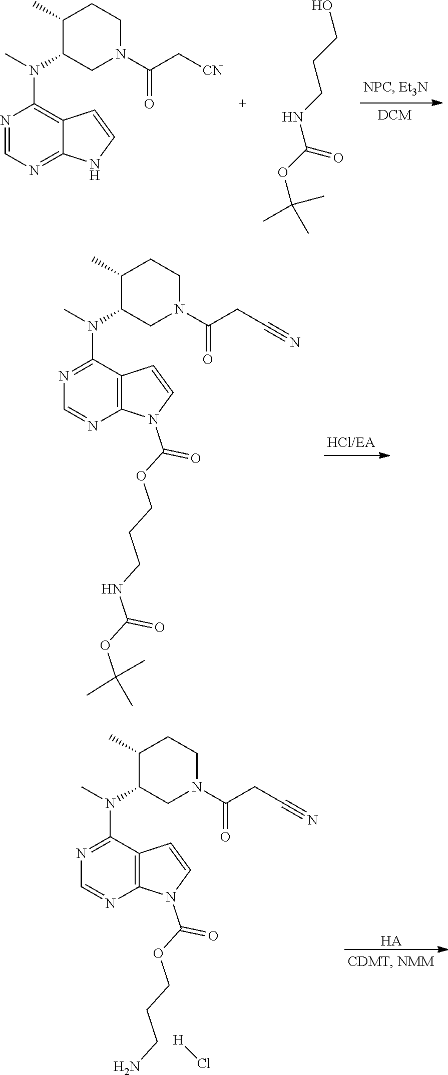

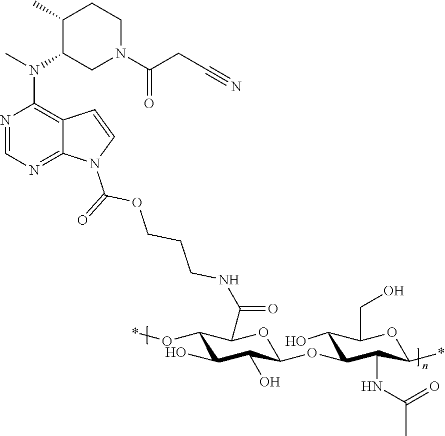

### Step 1: Preparation of 3-(tert-butoxycarbonylamino)propyl 4-[[(3R,4R)-1-(2-cyano acetyl)-4-methyl-3-piperidyl]-methyl-amino]pyrrolo[2,3-d]pyrimidine-7-carboxylate

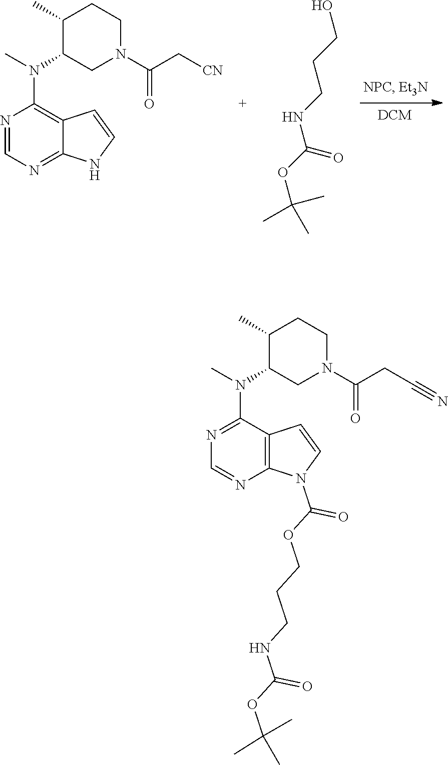 [SMILES: C[C@@H]1CCN(C(=O)CC#N)C[C@@H]1N(C)c1ncnc2[nH]ccc12 ; CC(C)(C)OC(=O)NCCCO ; C[C@@H]1CCN(C(=O)CC#N)C[C@@H]1N(C)c1ncnc2c1ccn2C(=O)OCCCNC(=O)OC(C)(C)C]

Following Step 1 of Example 1, tofacitinib (1249.5 mg, 4 mmol) and 3-(Boc-amino)-1-propanol (841.1 mg, 4.8 mmol) afforded the title compound (1.03 g, Yield: 50%). MS (m/z): [M+H]+ calcd for C₂₅H₃₅N₇O₅, 514.26; found, 514.2. ¹H NMR (400 MHz, CDCl₃) δ ppm 8.53 (d, J=10.4 Hz, 1H), 7.53 (dd, J=10.8, 4.0 Hz, 1H), 7.15 (s, 1H), 6.67 (d, J=4.1 Hz, 1H), 5.12 (d, J=30.4 Hz, 1H), 4.57 (dd, J=14.1, 8.5 Hz, 2H), 4.06-3.78 (m, 2H), 3.67-3.48 (m, 4H), 3.47-3.41 (m, 2H), 3.41-3.33 (m, 3H), 2.56-2.36 (m, 1H), 2.09-2.01 (m, 1H), 1.93 (tdd, J=22.0, 8.8, 4.5 Hz, 1H), 1.83-1.65 (m, 2H), 1.45 (s, 9H), 1.07 (dd, J=16.9, 7.1 Hz, 3H).

### Step 2: Preparation of 3-aminopropyl 4-[[(3R,4R)-1-(2-cyanoacetyl)-4-methyl-3-piperidyl]-methyl-amino]pyrrolo[2,3-d]pyrimidine-7-carboxylate hydrochloride

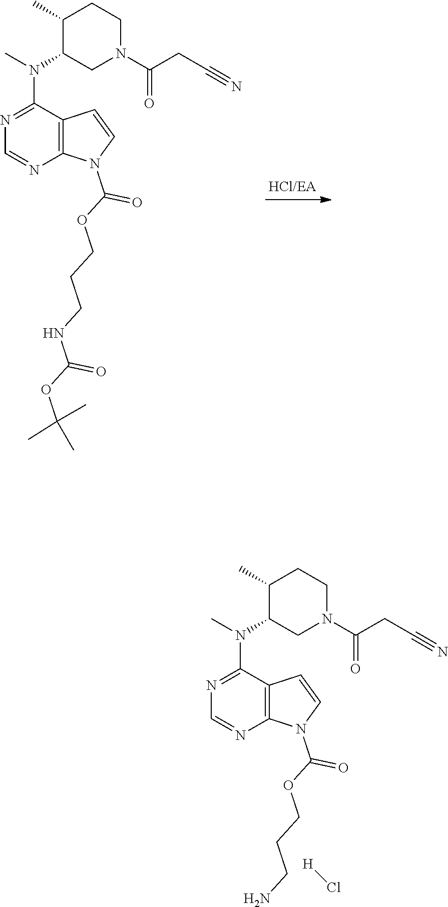

Following Step 2 of Example 1, 3-(tert-butoxycarbonylamino)propyl 4-[[(3R,4R)-1-(2-cyanoacetyl)-4-methyl-3-piperidyl]-methyl-amino]pyrrolo[2,3-d]pyrimidine-7-carboxylate (1.03 g, 2 mmol) afforded the title compound as a white solid (0.9 g, 100%). MS (m/z): [M+H]+ calcd for C₂₀H27N₇O₃, 414.21; found, 414.3.

### Step 3: Preparation of conjugate of 3-aminopropyl 4-[[(3R,4R)-1-(2-cyano acetyl)-4-methyl-3-piperidyl]-methyl-amino]pyrrolo[2,3-d]pyrimidine-7-carboxylate and HA

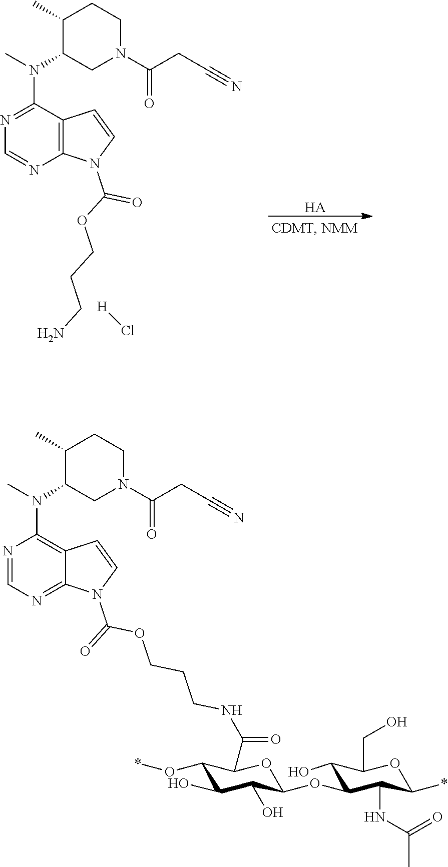

Following Step 3 of Example 1, sodium hyaluronate (MW 50 KDa, 165.6 mg, 0.411 mmol) and 3-aminopropyl-4-[[(3R,4R)-1-(2-cyanoacetyl)-4-methyl-3-piperidyl]-methyl-amino]pyrrolo[2,3-d]pyrimidine-7-carboxylate hydrochloride (200 mg, 0.411 mmol) afforded the title compound 0.162 g, Yield: 45.2%, DSR: 15%; ¹H NMR (400 MHz, D₂O) δ ppm 8.34-8.20 (m, 0.15H), 7.63-7.50 (m, 0.15H), 6.91-6.78 (m, 0.15H), 4.49-4.31 (m, 2.15H), 4.06-3.07 (m, 11.95H), 2.44-2.35 (m, 0.15H), 2.16-1.56 (m, 3.3H), 1.21 (t, J=7.0 Hz, 0.15H), 1.05-0.90 (m, 0.45H).

With Step 3 of Example 1, reaction of sodium hyaluronate (MW 2000 KDa) provided corresponding product (0.161 g, Yield: 44.9%, DSR: 17%).

### Example 35

### Preparation of conjugate of (4-aminocyclohexyl) 4-[[(3R,4R)-1-(2-cyano acetyl)-4-methyl-3-piperidyl]-methyl-amino]pyrrolo[2,3-d]pyrimidine-7-carboxylate and HA

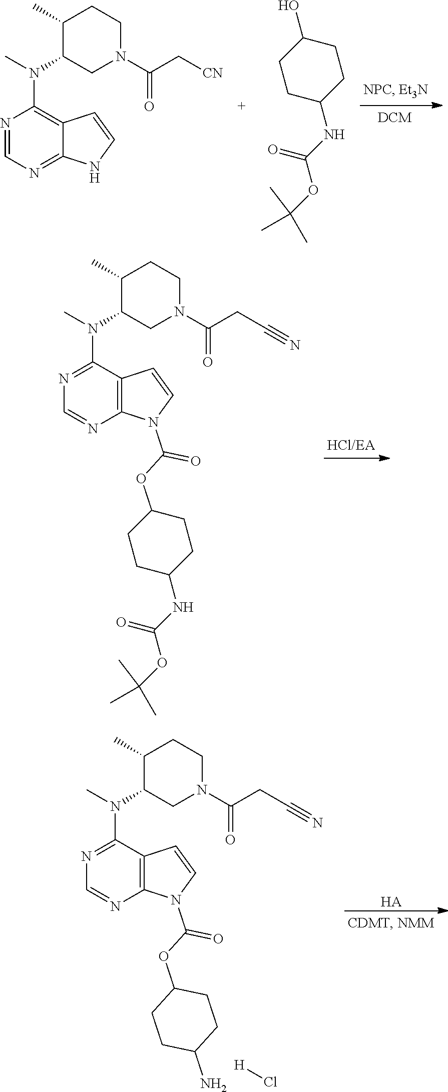

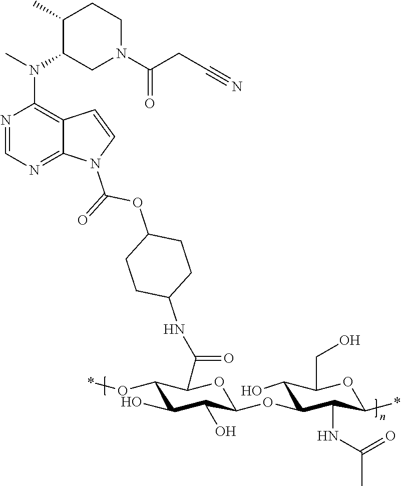

### Step 1: Preparation of [4-(tert-butoxycarbonylamino)cyclohexyl] 4-[[(3R,4R)-1-(2-cyanoacetyl)-4-methyl-3-piperidyl]-methyl-amino]pyrrolo[2,3-d]pyrimidine-7-carboxylate

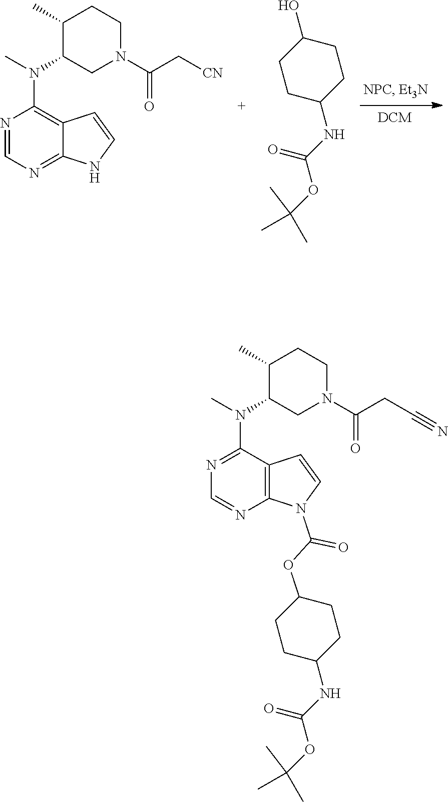 [SMILES: C[C@@H]1CCN(C(=O)CC#N)C[C@@H]1N(C)c1ncnc2[nH]ccc12 ; CC(C)(C)OC(=O)NC1CCC(O)CC1 ; C[C@@H]1CCN(C(=O)CC#N)C[C@@H]1N(C)c1ncnc2c1ccn2C(=O)OC1CCC(NC(=O)OC(C)(C)C)CC1]

Following Step 1 of Example 1, tofacitinib (1249.5 mg, 4 mmol) and tert-Butyl (4-hydroxycyclohexyl)carbamate (1033.4 mg, 4.8 mmol) afforded the title compound (0.851 g, Yield: 45%). MS (m/z): [M+H]+ calcd for C₂₈H₃₉N₇O₅, 554.30; found, 554.1. ¹H NMR (400 MHz, CDCl₃) δ ppm 8.46 (d, J=5.8 Hz, 1H), 7.42 (dd, J=9.5, 4.2 Hz, 1H), 6.61 (d, J=4.1 Hz, 1H), 5.19-5.04 (m, 1H), 5.03-4.89 (m, 1H), 4.45 (s, 1H), 4.08-3.76 (m, 2H), 3.63-3.46 (m, 4H), 3.36 (d, J=16.4 Hz, 3H), 2.49 (ddd, J=24.3, 11.9, 6.4 Hz, 1H), 2.26-2.06 (m, 4H), 1.93 (dddd, J=18.5, 13.7, 8.9, 4.4 Hz, 1H), 1.82-1.59 (m, 4H), 1.45 (s, 9H), 1.34 (td, J=13.3, 2.7 Hz, 2H), 1.15-0.99 (m, 3H).

### Step 2: Preparation of (4-aminocyclohexyl) 4-[[(3R,4R)-1-(2-cyanoacetyl)-4-methyl-3-piperidyl]-methyl-amino]pyrrolo[2,3-d]pyrimidine-7-carboxylate hydrochloride

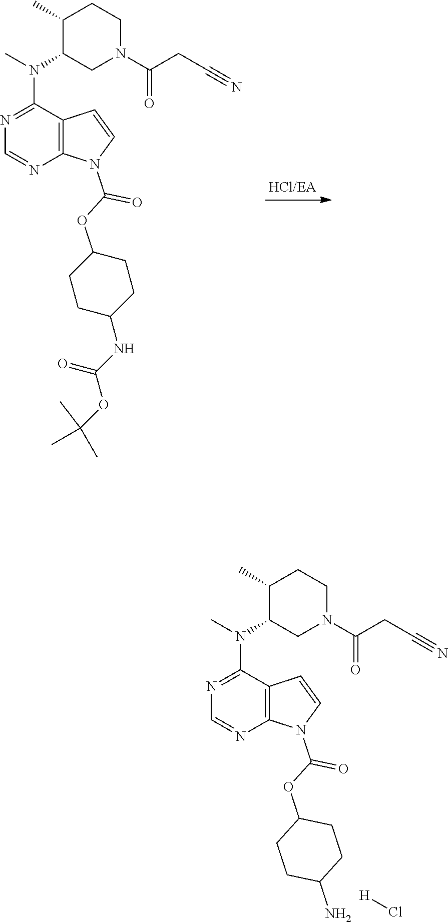

Following Step 2 of Example 1, [4-(tert-butoxycarbonylamino)cyclohexyl]-4-[[(3R,4R)-1-(2-cyanoacetyl)-4-methyl-3-piperidyl]-methyl-amino]pyrrolo[2,3-d]pyrimidine-7-carboxylate (0.62 g, 1.12 mmol) afforded the title compound as a white solid (0.55 g, Yield: 100%). MS (m/z): [M+H]+ calcd for C₂₃H₃₁N₇O₃, 454.24; found, 454.3.

### Step 3: Preparation of conjugate of (4-aminocyclohexyl) 4-[[(3R,4R)-1-(2-cyanoacetyl)-4-methyl-3-piperidyl]-methyl-amino]pyrrolo[2,3-d]pyrimidine-7-carboxylate and HA

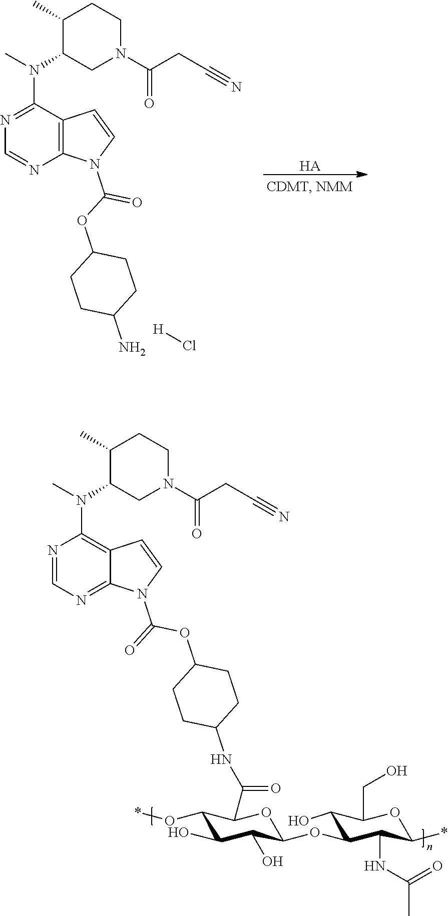

Following Step 3 of Example 1, sodium hyaluronate (MW 50 KDa, 153 mg, 0.38 mmol) and (4-aminocyclohexyl)4-[[(3R,4R)-1-(2-cyanoacetyl)-4-methyl-3-piperidyl]-methyl-amino]pyrrolo[2,3-d]pyrimidine-7-carboxylate hydrochloride (200 mg, 0.38 mmol) afforded the title compound 0.173 g, Yield: 49.9%, DSR: 11%; ¹H NMR (400 MHz, D₂O) δ ppm 8.24-8.02 (m, 0.11H), 7.57-7.37 (m, 0.11H), 6.80-6.64 (m, 0.11H), 4.96-4.86 (m, 0.11H), 4.57-4.27 (m, 2H), 4.07-3.04 (m, 10.99H), 2.46-2.30 (m, 0.11H), 2.27-1.76 (m, 3.77H), 1.74-1.33 (m, 0.66H), 1.22 (d, J=6.9 Hz, 0.11H), 1.09-0.85 (m, 0.33H).

With Step 3 of Example 1, reaction of sodium hyaluronate (MW 2000 KDa) provided corresponding product (0.162 g, Yield: 46.7%, DSR: 27%).

### Example 36

### Preparation of conjugate of 4-piperidyl 4-[[(3R,4R)-1-(2-cyanoacetyl)-4-methyl-3-piperidyl]-methyl-amino]pyrrolo[2,3-d]pyrimidine-7-carboxylate and HA

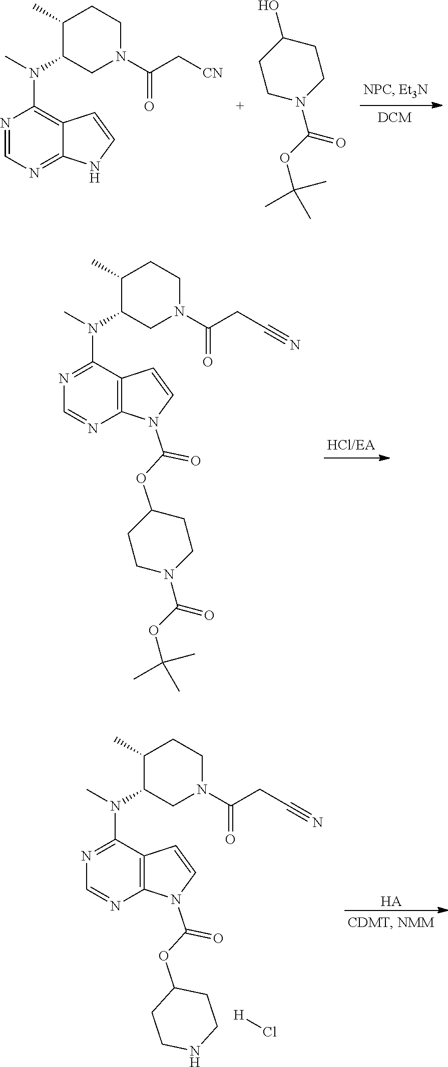

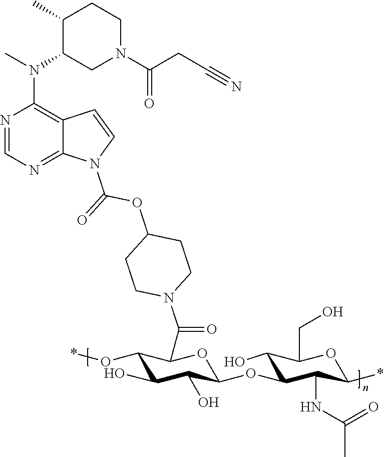

### Step 1: Preparation of (1-tert-butoxycarbonyl-4-piperidyl) 4-[[(3R,4R)-1-(2-cyanoacetyl)-4-methyl-3-piperidyl]-methyl-amino]pyrrolo[2,3-d]pyrimidine-7-carboxylate

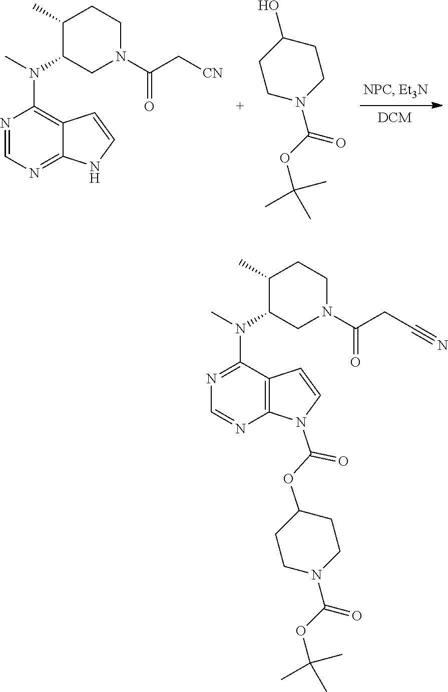

Following Step 1 of Example 1, tofacitinib (1249.5 mg, 4 mmol) and tert-butyl 4-hydroxypiperidine-1-carboxylate (966 mg, 4.8 mmol) afforded the title compound (0.537 g, Yield: 24.9%). MS (m/z): [M+H]+ calcd for C₂₇H₃₇N₇O₅, 540.28; found, 540.2.

### Step 2: Preparation of 4-piperidyl 4-[[(3R,4R)-1-(2-cyanoacetyl)-4-methyl-3-piperidyl]-methyl-amino]pyrrolo[2,3-d]pyrimidine-7-carboxylate hydrochloride

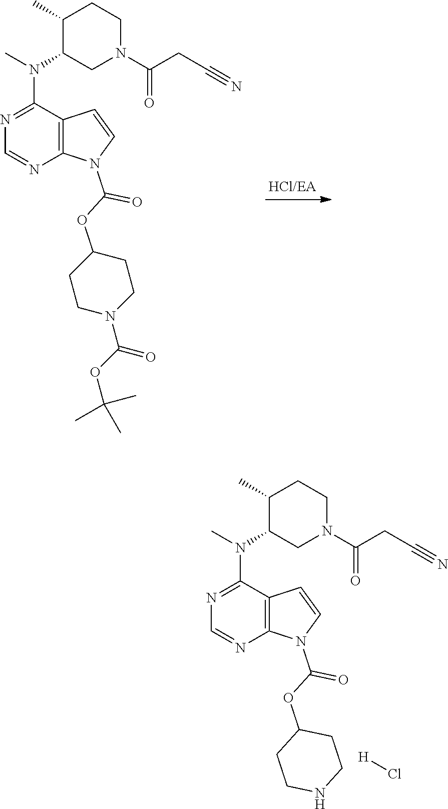

Following Step 2 of Example 1, (1-tert-butoxycarbonyl-4-piperidyl) 4-[[(3R,4R)-1-(2-cyano acetyl)-4-methyl-3-piperidyl]-methyl-amino]pyrrolo[2,3-d]pyrimidine-7-carboxylate (0.47 g, 0.87 mmol afforded the title compound as a white solid 0.25 g, Yield: 58%). MS (m/z): [M+H]+ calcd for C₂₂H₂₉N₇O₃, 440.23; found, 440.3.

### Step 3: Preparation of conjugate of 4-piperidyl 4-[[(3R,4R)-1-(2-cyanoacetyl)-4-methyl-3-piperidyl]-methyl-amino]pyrrolo[2,3-d]pyrimidine-7-carboxylate and HA

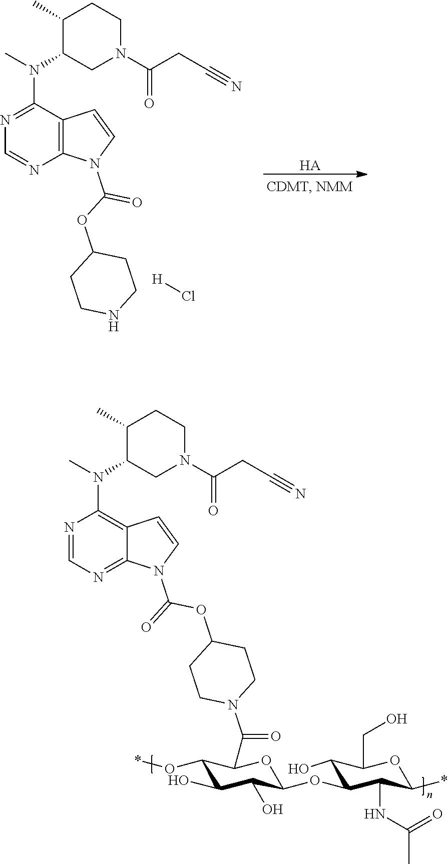

Following Step 3 of Example 1, sodium hyaluronate (MW 50 KDa, 169.7 mg, 0.421 mmol) and 4-piperidyl-4-[[(3R,4R)-1-(2-cyanoacetyl)-4-methyl-3-piperidyl]-methyl-amino]pyrrolo[2,3-d]pyrimidine-7-carboxylate hydrochloride (200 mg, 0.421 mmol) afforded the title compound 0.161 g, Yield: 44.4%, DSR: 11%; ¹H NMR (400 MHz, D₂O) δ ppm 8.27-8.04 (m, 0.11H), 7.61-7.38 (m, 0.11H), 6.84-6.68 (m, 0.11H), 4.61-4.28 (m, 2.11H), 4.12-3.05 (m, 11.43H), 2.41-2.30 (m, 0.11H), 2.19-1.58 (m, 3.66H), 1.20 (t, J=6.9 Hz, 0.11H), 1.08-0.85 (m, 0.33H).

### Example 37

### Preparation of conjugate of 2-(2-aminoethoxy)ethyl 4-[[(3R,4R)-1-(2-cyanoacetyl)-4-methyl-3-piperidyl]-methyl-amino]pyrrolo[2,3-d]pyrimidine-7-carboxylate and HA

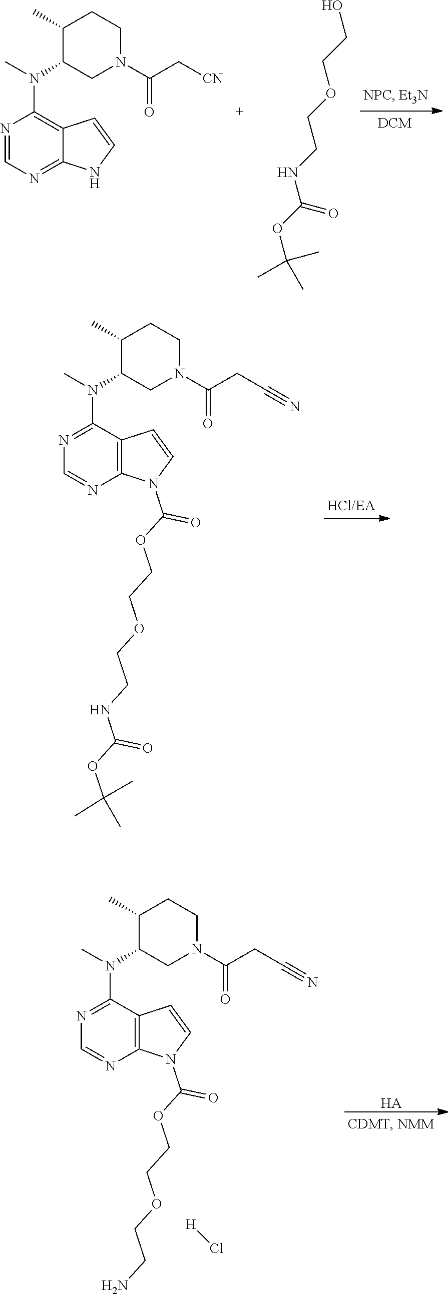

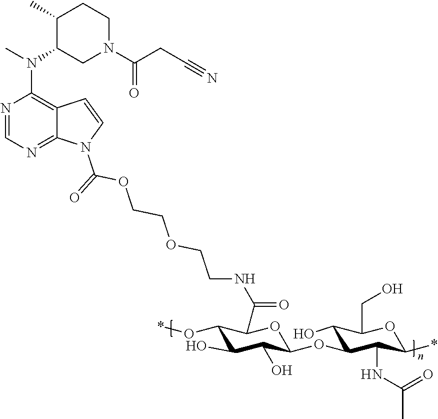

### Step 1: Preparation of 2-[2-(tert-butoxycarbonylamino)ethoxy]ethyl 4-[[(3R,4R)-1-(2-cyanoacetyl)-4-methyl-3-piperidyl]-methyl-amino]pyrrolo[2,3-d]pyrimidine-7-carboxylate

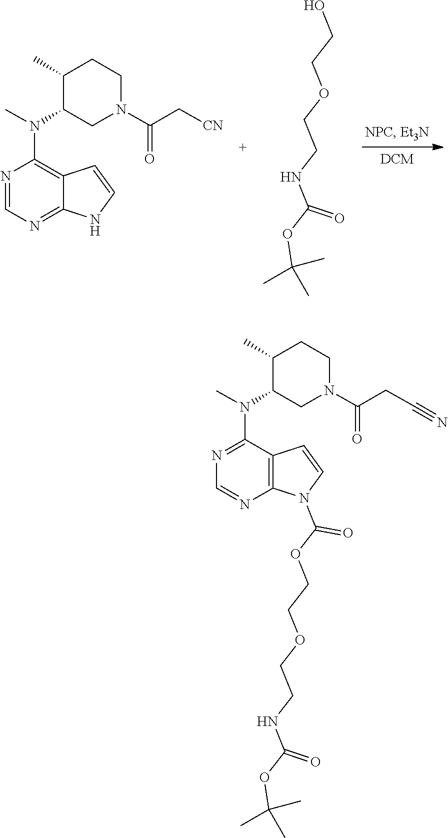 [SMILES: C[C@@H]1CCN(C(=O)CC#N)C[C@@H]1N(C)c1ncnc2[nH]ccc12 ; CC(C)(C)OC(=O)NCCOCCO ; C[C@@H]1CCN(C(=O)CC#N)C[C@@H]1N(C)c1ncnc2c1ccn2C(=O)OCCOCCNC(=O)OC(C)(C)C]

To a stirred mixture of tofacitinib (1250 mg, 4 mmol) and bis(4-nitrophenyl) carbonate (1460 mg, 4.8 mmol) in dichloromethane (40 mL) was added triethylamine (1.1 mL, 8 mmol). The reaction mixture was heated to 45° C. and stirred at this temperature for 6 h. Then the reaction mixture was cooled to room temperature. tert-Butyl (2-(2-hydroxyethoxy)ethyl)carbamate (985.2 mg, 4.8 mmol) was added. The reaction mixture was stirred at room temperature for 16 h. The solution was diluted with dichloromethane and washed with saturated NaHCO₃ solution, water and saturated brine solution. The organic layer was dried over sodium sulfate, filtered and then concentrated under reduced pressure to afford the title compound (1.3 g, Yield: 59.8%). MS (m/z): [M+H]+ calcd for C₂₆H₃₇N₇O₆, 544.28; found, 544.2.

### Step 2: Preparation of 2-(2-aminoethoxy)ethyl 4-[[(3R,4R)-1-(2-cyanoacetyl)-4-methyl-3-piperidyl]-methyl-amino]pyrrolo[2,3-d]pyrimidine-7-carboxylate hydrochloride

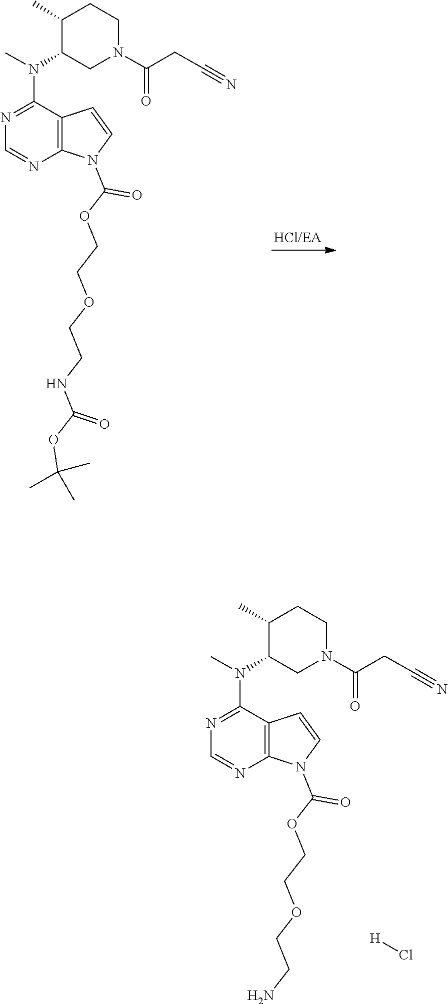 [SMILES: C[C@@H]1CCN(C(=O)CC#N)C[C@@H]1N(C)c1ncnc2c1ccn2C(=O)OCCOCCNC(=O)OC(C)(C)C ; C[C@@H]1CCN(C(=O)CC#N)C[C@@H]1N(C)c1ncnc2c1ccn2C(=O)OCCOCCN ; Cl]

Following Step 2 of Example 1, 2-[2-(tert-butoxycarbonylamino)ethoxy]ethyl 4-[[(3R,4R)-1-(2-cyanoacetyl)-4-methyl-3-piperidyl]-methyl-amino]pyrrolo[2,3-d]pyrimidine-7-carboxylate (1.3 g, 2.39 mmol) afforded the title compound as a white solid (1.1 g, Yield: 95.9%). MS (m/z): [M+H]+ calcd for C₂₁H₂₉N₇O₄, 444.22; found, 444.2.

### Step 3: Preparation of conjugate of 2-(2-aminoethoxy)ethyl 4-[[(3R,4R)-1-(2-cyanoacetyl)-4-methyl-3-piperidyl]-methyl-amino]pyrrolo[2,3-d]pyrimidine-7-carboxylate and HA

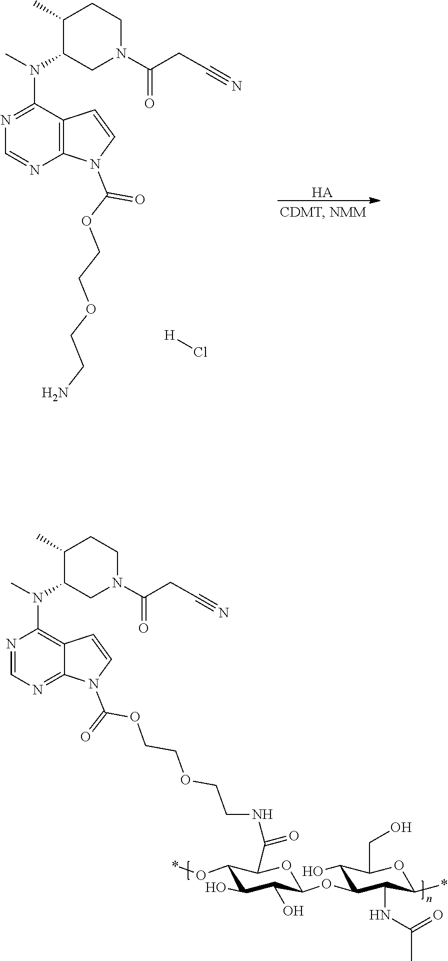

Following Step 3 of Example 1, sodium hyaluronate (MW 50 KDa, 156.4 mg, 0.388 mmol) and 2-(2-aminoethoxy)ethyl4-[[(3R,4R)-1-(2-cyanoacetyl)-4-methyl-3-piperidyl]-methyl-amino]pyrrolo[2,3-d]pyrimidine-7-carboxylate hydrochloride (200 mg, 0.388 mmol) afforded the title compound 0.175 g, Yield: 50%, DSR: 15%; ¹H NMR (400 MHz, D₂O) δ ppm 8.23-8.09 (m, 0.15H), 7.58-7.39 (m, 0.15H), 6.86-6.65 (m, 0.15H), 4.58-4.25 (m, 2.15H), 4.02-2.94 (m, 12.55H), 2.43-2.28 (m, 0.15H), 2.16-1.56 (m, 3.3H), 1.10-0.89 (m, 0.45H).

With Step 3 of Example 1, reaction of sodium hyaluronate (MW 2000 KDa) provided corresponding product (0.182 g, Yield: 52%, DSR: 17%).

### Example 38

### Preparation of conjugate of methyl (2S)-6-amino-2-[4-[[4-[[(3R,4R)-1-(2-cyanoacetyl)-4-methyl-3-piperidyl]-methyl-amino]pyrrolo[2,3-d]pyrimidine-7-carbonyl]amino]butanoylamino]hexanoate

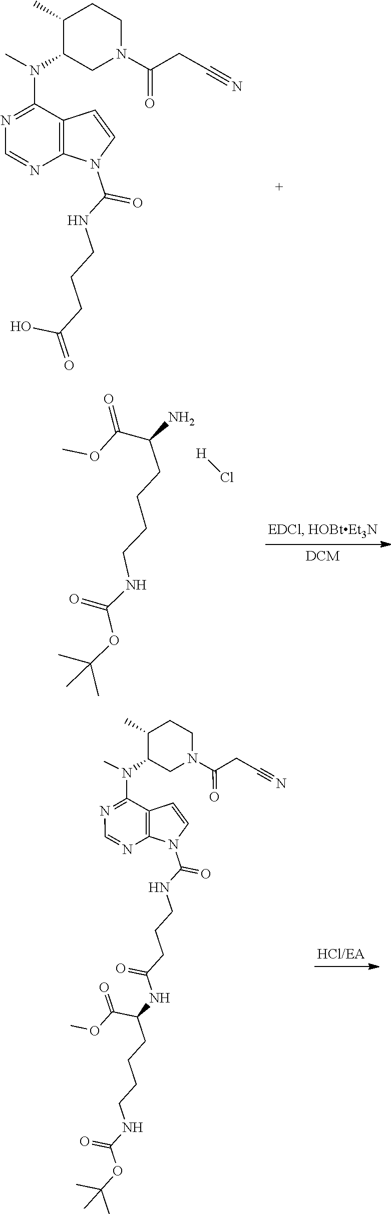

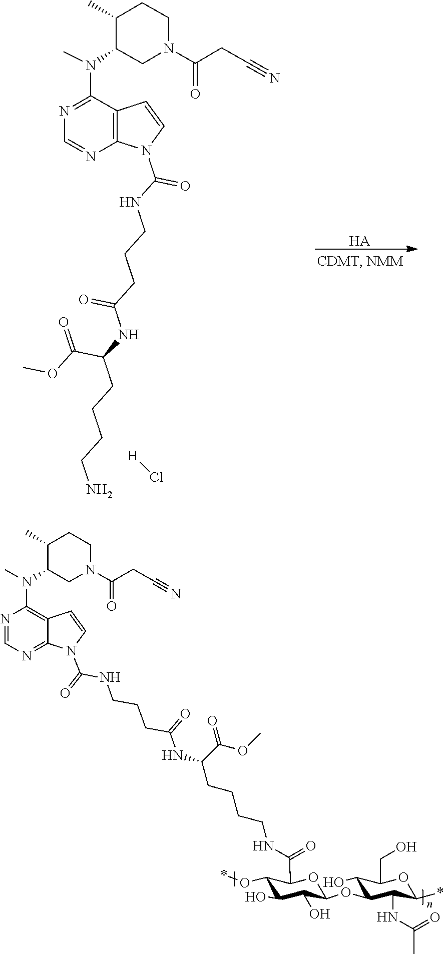

### Step 1: Preparation of methyl (2S)-6-(tert-butoxycarbonylamino)-2-[4-[[4-[[(3R,4R)-1-(2-cyanoacetyl)-4-methyl-3-piperidyl]-methyl-amino]pyrrolo[2,3-d]pyrimidine-7-carbonyl]amino]butanoylamino]hexanoate

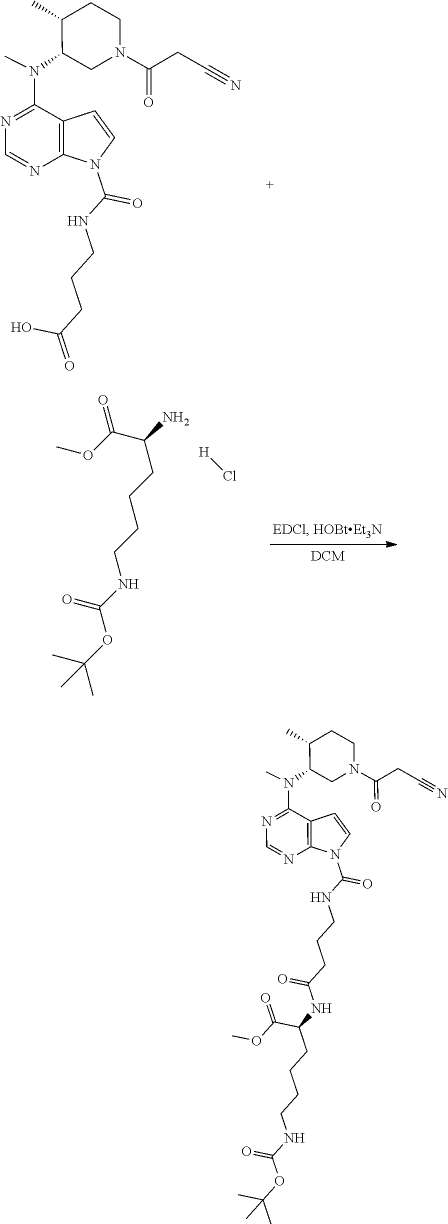 [SMILES: C[C@@H]1CCN(C(=O)CC#N)C[C@@H]1N(C)c1ncnc2c1ccn2C(=O)NCCCC(=O)O ; COC(=O)[C@@H](N)CCCCNC(=O)OC(C)(C)C ; Cl ; COC(=O)[C@H](CCCCNC(=O)OC(C)(C)C)NC(=O)CCCNC(=O)n1ccc2c(N(C)[C@H]3CN(C(=O)CC#N)CC[C@H]3C)ncnc21]

4-[[4-[[(3R,4R)-1-(2-cyanoacetyl)-4-methyl-3-piperidyl]-methyl-amino]pyrrolo[2,3-d]pyrimidine-7-carbonyl]amino]butanoic acid (1323 mg, 3 mmol), (S)-methyl 6-amino-2-((tert-butoxycarbonyl)-amino)hexanoate (1336 mg, 4.5 mmol), 1-ethyl-3-(3-dimethylaminopropyl)carbodiimide (EDCI, 863 mg, 4.5 mmol) and 1-hydroxybenzotriazole (HOBt, 608 mg, 4.5 mmol) were dissolved in dichloromethane (40 mL) and triethylamine (2.08 mL, 15 mmol) was added. The reaction mixture was stirred at room temperature for 16 h. The solution was diluted with dichloromethane and washed with water and saturated brine solution. The organic layer was dried over sodium sulfate, filtered and then concentrated under reduced pressure. The crude residue was purified by column chromatography (Methanol/dichloromethane=1:150-1:80) to afford the title compound (1.726 g, Yield: 83.3%). MS (m/z): [M+H]+ calcd for C₃₃H₄₉N₉O₇, 684.37; found, 684.1. ¹H NMR (400 MHz, CDCl₃) δ ppm 9.89 (dd, J=13.5, 7.5 Hz, 1H), 8.29 (d, J=9.0 Hz, 1H), 7.72 (dd, J=11.9, 4.0 Hz, 1H), 6.75-6.50 (m, 2H), 5.14 (s, 1H), 4.75-4.48 (m, 2H), 4.12-3.76 (m, 2H), 3.73-3.32 (m, 9H), 3.11 (d, J=5.4 Hz, 2H), 2.58-2.42 (m, 1H), 2.41-2.25 (m, 2H), 2.09-1.65 (m, 6H), 1.54-1.32 (m, 15H), 1.10 (dd, J=12.7, 7.1 Hz, 3H).

### Step 2: Preparation of methyl (2S)-6-amino-2-[4-[[4-[[(3R,4R)-1-(2-cyanoacetyl)-4-methyl-3-piperidyl]-methyl-amino]pyrrolo[2,3-d]pyrimidine-7-carbonyl]amino]butanoylamino]hexanoate hydrochloride

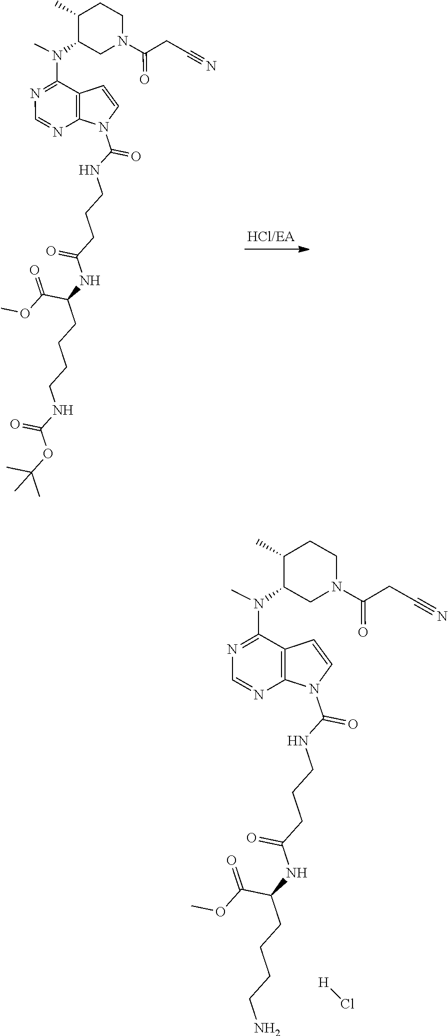 [SMILES: COC(=O)[C@H](CCCCNC(=O)OC(C)(C)C)NC(=O)CCCNC(=O)n1ccc2c(N(C)[C@H]3CN(C(=O)CC#N)CC[C@H]3C)ncnc21 ; COC(=O)[C@H](CCCCN)NC(=O)CCCNC(=O)n1ccc2c(N(C)[C@H]3CN(C(=O)CC#N)CC[C@H]3C)ncnc21 ; Cl]

Following Step 2 of Example 1, methyl (2S)-6-(tert-butoxycarbonylamino)-2-[4-[[4-[[(3R,4R)-1-(2-cyanoacetyl)-4-methyl-3-piperidyl]-methyl-amino]pyrrolo[2,3-d]pyrimidine-7-carbonyl]amino]butanoylamino]hexanoate (1.5 g, 2.137 mmol) afforded the title compound as a white solid (1.426 g, yield: 99.6%). MS (m/z): [M+H]+ calcd for C₂₈H₄₁N₉O₅, 584.32; found, 584.1.

### Step 3: Preparation of conjugate of methyl (2S)-6-amino-2-[4-[[4-[[(3R,4R)-1-(2-cyanoacetyl)-4-methyl-3-piperidyl]-methyl-amino]pyrrolo[2,3-d]pyrimidine-7-carbonyl]amino]butanoylamino]hexanoate

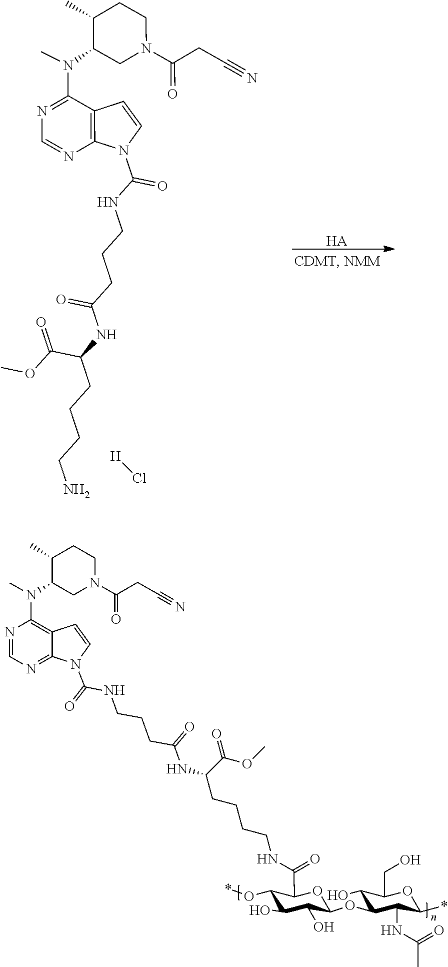

Following Step 3 of Example 1, sodium hyaluronate (MW 50 KDa, 123 mg, 0.305 mmol) and methyl(2S)-6-amino-2-[4-[[4-[[(3R,4R)-1-(2-cyanoacetyl)-4-methyl-3-piperidyl]-methyl-amino]pyrrolo[2,3-d]pyrimidine-7-carbonyl]amino]butanoylamino]hexanoate hydrochloride (200 mg, 0.36 mmol) afforded the title compound 0.13 g, Yield: 45%, DSR: 5%; ¹H NMR (400 MHz, D₂O) δ ppm 8.29-8.16 (m, 0.05H), 7.64-7.51 (m, 0.05H), 6.82-6.65 (m, 0.05H), 4.57-4.22 (m, 2.05H), 3.99-2.89 (m, 10.75H), 2.42-2.29 (m, 0.1H), 2.14-1.55 (m, 3.3H), 1.47-1.35 (m, 0.3H), 1.24-1.11 (m, 0.15H).

### Example 39

### Preparation of conjugate of N-[4-(2-aminoethylamino)-4-oxo-butyl]-4-[[(3R,4R)-1-(2-cyanoacetyl)-4-methyl-3-piperidyl]-methyl-amino]pyrrolo[2,3-d]pyrimidine-7-carboxamide and HA

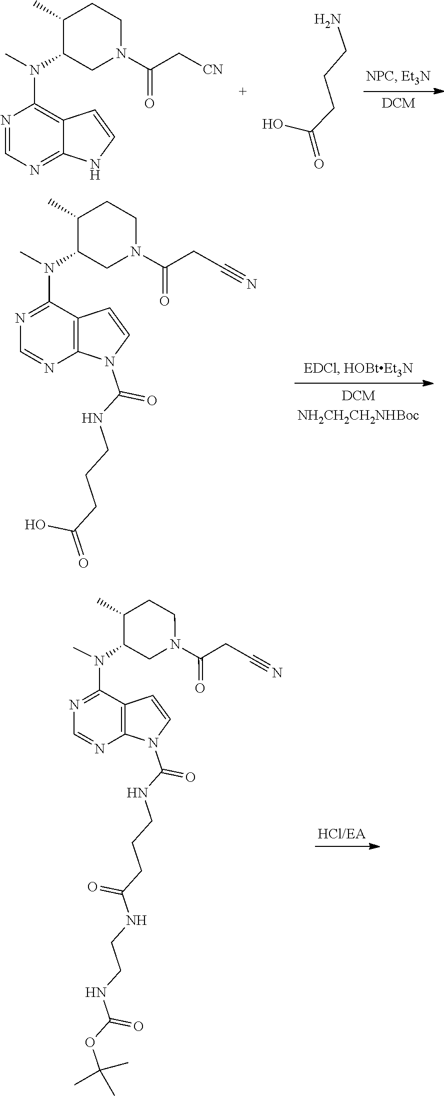

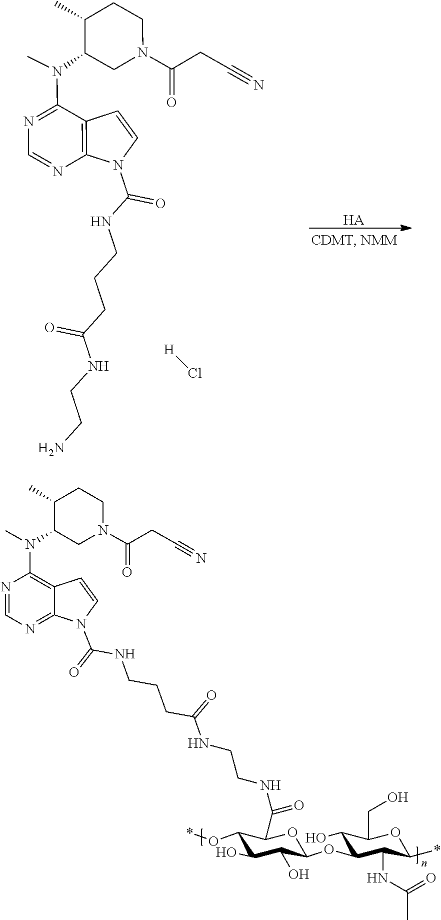

### Step 1: Preparation of 4-[[4-[[(3R,4R)-1-(2-cyanoacetyl)-4-methyl-3-piperidyl]-methyl-amino]pyrrolo[2,3-d]pyrimidine-7-carbonyl]amino]butanoic acid

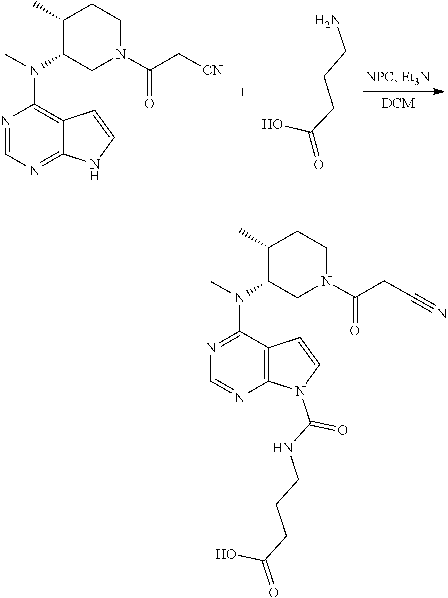 [SMILES: C[C@@H]1CCN(C(=O)CC#N)C[C@@H]1N(C)c1ncnc2[nH]ccc12 ; NCCCC(=O)O ; C[C@@H]1CCN(C(=O)CC#N)C[C@@H]1N(C)c1ncnc2c1ccn2C(=O)NCCCC(=O)O]

To a stirred mixture of tofacitinib (30 g, 96.1 mmol) and bis(4-nitrophenyl) carbonate (43.3 g, 142.4 mmol) in dichloromethane (300 mL) was added triethylamine (33.9 g, 336.3 mmol). The reaction mixture was heated to 45° C. and stirred at this temperature for 16 h. Then the reaction mixture was cooled to 0˜110° C. 300 mL aqueous solution of 4-aminobutyric acid (19.8 g, 192.2 mmol), triethylamine (19.4 g, 192.2 mmol) and tetrabutylammonium bromide (0.3 g) were added. The reaction mixture was stirred at 0˜10° C. for 16 h, the pH value of the reaction mixture was adjusted to 3-4 with 20% citric acid aqueous solution and the organic layer was washed with 10% citric acid aqueous solution, water and saturated brine solution. The organic layer was dried over sodium sulfate, filtered and then concentrated under reduced pressure. The crude residue was purified by column chromatography (methanol/dichloromethane=1:00˜1:50) to afford the title compound (10.3 g, 24%). MS (m/z): [M+H]+ calcd for C₂₁H₂₇N₇O₄, 442.2; found, 442.3.

### Step 2: Preparation of tert-butyl N-[2-[4-[[4-[[(3R,4R)-1-(2-cyanoacetyl)-4-methyl-3-piperidyl]-methyl-amino]pyrrolo[2,3-d]pyrimidine-7-carbonyl]amino]butanoylamino]ethyl]carbamate

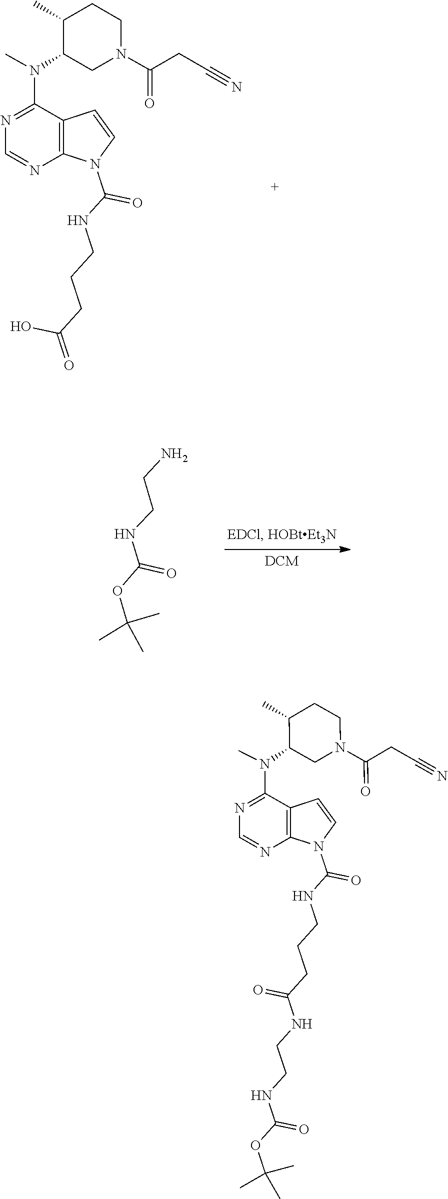 [SMILES: C[C@@H]1CCN(C(=O)CC#N)C[C@@H]1N(C)c1ncnc2c1ccn2C(=O)NCCCC(=O)O ; CC(C)(C)OC(=O)NCCN ; C[C@@H]1CCN(C(=O)CC#N)C[C@@H]1N(C)c1ncnc2c1ccn2C(=O)NCCCC(=O)NCCNC(=O)OC(C)(C)C]

4-[[4-[[(3R,4R)-1-(2-cyanoacetyl)-4-methyl-3-piperidyl]-methyl-amino]pyrrolo[2,3-d]pyrimidine-7-carbonyl]amino]butanoic acid (1323 mg, 3 mmol), N-Boc-ethylenediamine (721 mg, 4.5 mmol), 1-ethyl-3-(3-dimethylaminopropyl) carbodiimide (EDCI, 863 mg, 4.5 mmol) and 1-hydroxybenzotriazole (HOBt, 607.5 mg, 4.5 mmol.) were dissolved in dichloromethane (40 mL) and triethylamine (1.25 mL, 9 mmol) was added. The reaction mixture was stirred at room temperature for 16 h. The solution was diluted with dichloromethane and washed with water and saturated brine solution. The organic layer was dried over sodium sulfate, filtered and then concentrated under reduced pressure. The crude residue was purified by column chromatography (methanol/dichloromethane=1:100-1:50) to afford the title compound (1.58 g, yield: 90%). MS (m/z): [M+H]+ calcd for C₂₈H₄₁N₉O₅, 584.32; found, 584.2. ¹H NMR (400 MHz, CDCl₃) δ ppm 10.02-9.67 (m, 1H), 8.29 (d, J=8.9 Hz, 1H), 7.72 (dd, J=10.5, 4.0 Hz, 1H), 6.58 (dd, J=14.2, 10.2 Hz, 2H), 5.32 (d, J=17.1 Hz, 1H), 5.13 (s, 1H), 4.20-3.70 (m, 2H), 3.69-3.44 (m, 6H), 3.39-3.14 (m, 7H), 2.60-2.40 (m, 1H), 2.26 (dt, J=15.3, 7.4 Hz, 2H), 2.11-1.83 (m, 3H), 1.83-1.70 (m, 1H), 1.41 (d, J=28.2 Hz, 9H), 1.09 (dd, J=12.6, 7.1 Hz, 3H).

### Step 3: Preparation of N-[4-(2-aminoethylamino)-4-oxo-butyl]-4-[[(3R,4R)-1-(2-cyanoacetyl)-4-methyl-3-piperidyl]-methyl-amino]pyrrolo[2,3-d]pyrimidine-7-carboxamide hydrochloride

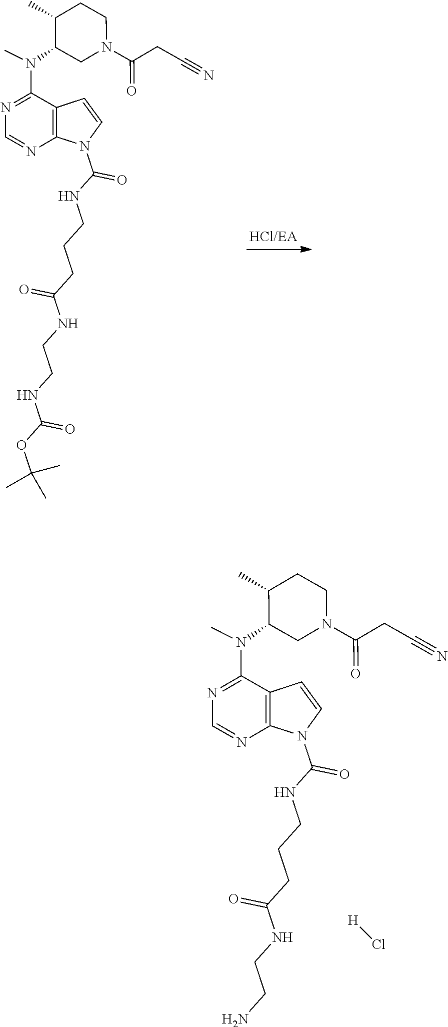 [SMILES: C[C@@H]1CCN(C(=O)CC#N)C[C@@H]1N(C)c1ncnc2c1ccn2C(=O)NCCCC(=O)NCCNC(=O)OC(C)(C)C ; C[C@@H]1CCN(C(=O)CC#N)C[C@@H]1N(C)c1ncnc2c1ccn2C(=O)NCCCC(=O)NCCN ; Cl]

Following Step 2 of Example 1, tert-butyl N-[2-[4-[[4-[[(3R,4R)-1-(2-cyanoacetyl)-4-methyl-3-piperidyl]-methyl-amino]pyrrolo[2,3-d]pyrimidine-7-carbonyl]amino]butanoylamino]ethyl]carbamate (1.18 g, 2 mmol) afford the title compound as a white solid (1.04 g, yield: 100%). MS (m/z): [M+H]+ calcd for C₂₃H₃₃N₉O₃, 484.27; found, 484.3.

### Step 4: Preparation of conjugate of N-[4-(2-aminoethylamino)-4-oxo-butyl]-4-[[(3R,4R)-1-(2-cyanoacetyl)-4-methyl-3-piperidyl]-methyl-amino]pyrrolo[2,3-d]pyrimidine-7-carboxamide and HA

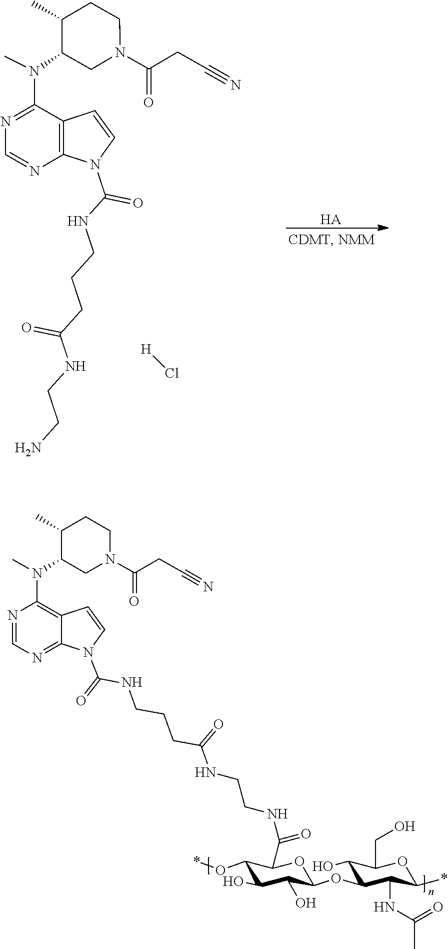

Following Step 3 of Example 1, sodium hyaluronate (MW 50 KDa, 145 mg, 0.36 mmol) and N-[4-(2-aminoethylamino)-4-oxo-butyl]-4-[[(3R,4R)-1-(2-cyanoacetyl)-4-methyl-3-piperidyl]-methyl-amino]pyrrolo[2,3-d]pyrimidine-7-carboxamide hydrochloride (200 mg, 0.36 mmol) afforded the title compound 0.174 g, Yield: 51.4%, DSR: 30%; ¹H NMR (400 MHz, D₂O) δ ppm 8.24-8.13 (m, 0.2H), 7.61-7.47 (m, 0.2H), 6.79-6.61 (m, 0.2H), 4.58-4.16 (m, 2.2H), 4.10-2.77 (m, 13H), 2.42-2.18 (m, 0.6H), 2.04-1.55 (m, 4H), 1.06-0.85 (m, 0.6H).

With Step 3 of Example 1, reaction of sodium hyaluronate (MW 500 KDa) provided corresponding product (0.186 g, Yield: 54.9%, DSR: 39%),

With Step 3 of Example 1, reaction of sodium hyaluronate (MW 2000 KDa) provided corresponding product (0.189 g, Yield: 55.8%, DSR: 30%.

### Example 40

### Preparation of conjugate of N-[4-(4-aminobutylamino)-4-oxo-butyl]-4-[[(3R,4R)-1-(2-cyanoacetyl)-4-methyl-3-piperidyl]-methyl-amino]pyrrolo[2,3-d]pyrimidine-7-carboxamide and HA

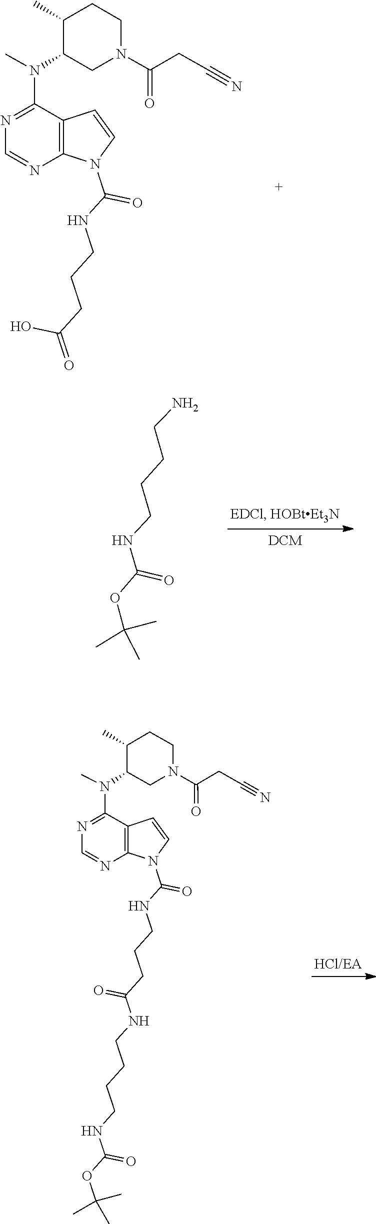 [SMILES: C[C@@H]1CCN(C(=O)CC#N)C[C@@H]1N(C)c1ncnc2c1ccn2C(=O)NCCCC(=O)O ; CC(C)(C)OC(=O)NCCCCN ; C[C@@H]1CCN(C(=O)CC#N)C[C@@H]1N(C)c1ncnc2c1ccn2C(=O)NCCCC(=O)NCCCCNC(=O)OC(C)(C)C]

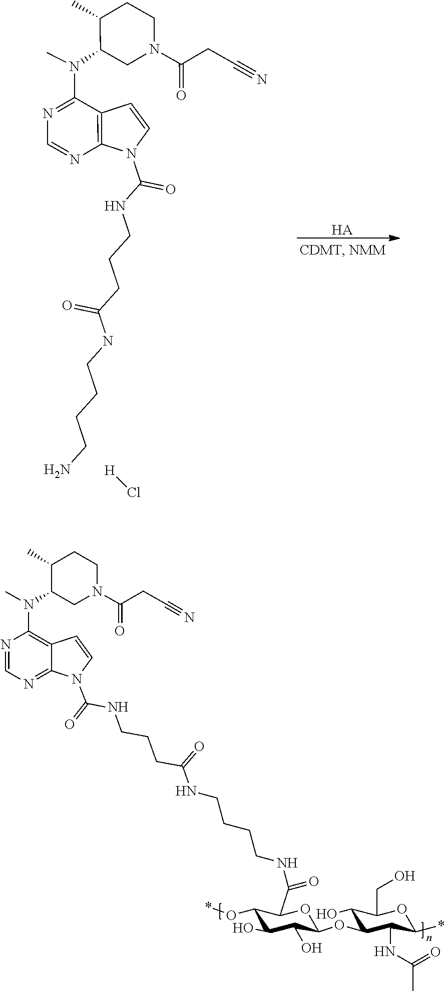

### Step 1: Preparation of tert-butyl N-[4-[4-[[4-[[(3R,4R)-1-(2-cyanoacetyl)-4-methyl-3-piperidyl]-methyl-amino]pyrrolo[2,3-d]pyrimidine-7-carbonyl]amino]butanoylamino]butyl]carbamate

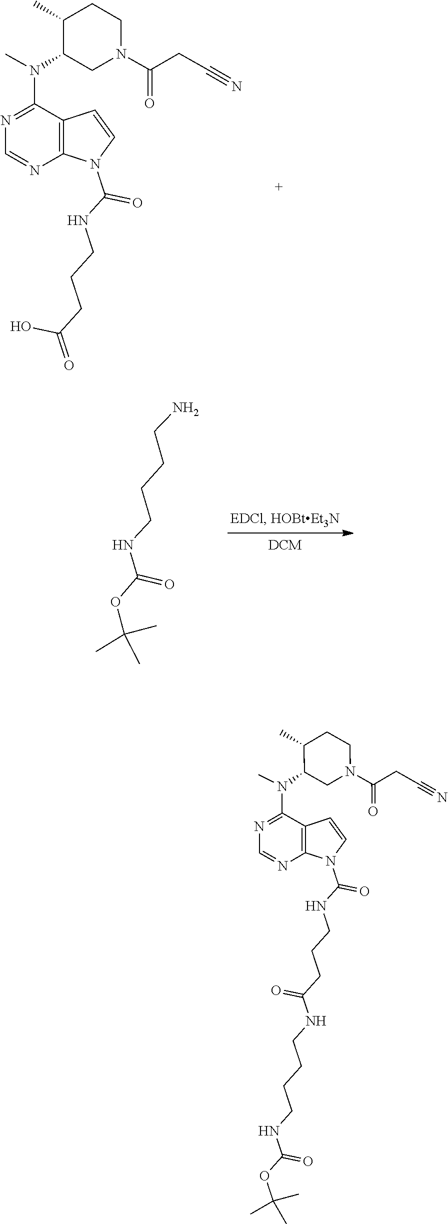 [SMILES: C[C@@H]1CCN(C(=O)CC#N)C[C@@H]1N(C)c1ncnc2c1ccn2C(=O)NCCCC(=O)O ; CC(C)(C)OC(=O)NCCCCN ; C[C@@H]1CCN(C(=O)CC#N)C[C@@H]1N(C)c1ncnc2c1ccn2C(=O)NCCCC(=O)NCCCCNC(=O)OC(C)(C)C]

4-[[4-[[(3R,4R)-1-(2-cyanoacetyl)-4-methyl-3-piperidyl]-methyl-amino]pyrrolo[2,3-d]pyrimidine-7-carbonyl]amino]butanoic acid (886 mg, 2 mmol), N-Boc-1,4-butanediamine (564 mg, 3 mmol), 1-ethyl-3-(3-dimethylamino propyl)carbodiimide (EDCI, 575.1 mg, 3 mmol) and 1-hydroxybenzotriazole (HOBt, 405 mg, 3 mmol) were dissolved in dichloromethane (30 mL) and triethylamine (0.84 mL, 6 mmol) was added. The reaction mixture was stirred at room temperature for 16 h. The solution was diluted with dichloromethane and washed with water and saturated brine solution. The organic layer was dried over sodium sulfate, filtered and then concentrated under reduced pressure. The crude residue was purified by column chromatography (methanol/dichloromethane=1:100˜1:50) to afford the title compound (0.52 g, yield: 42.5%). MS (m/z): [M+H]+ calcd for C₃₀H₄₅N₉O₅, 612.35; found, 612.1. ¹H NMR (400 MHz, d-DMSO) δ ppm 9.62 (s, 1H), 8.29 (t, J=14.4 Hz, 1H), 7.77 (s, 1H), 7.61 (d, J=22.9 Hz, 1H), 6.77 (d, J=28.6 Hz, 2H), 4.86 (s, 1H), 4.22-3.61 (m, 5H), 3.43-3.33 (m, 3H), 3.27 (s, 3H), 2.94 (d, J=25.8 Hz, 2H), 2.86 (s, 2H), 2.37 (s, 1H), 2.13 (t, J=7.0 Hz, 2H), 1.91-1.39 (m, 5H), 1.33 (s, 12H), 0.97 (t, J=14.9 Hz, 3H).

### Step 2: Preparation of N-[4-(4-aminobutylamino)-4-oxo-butyl]-4-[[(3R,4R)-1-(2-cyanoacetyl)-4-methyl-3-piperidyl]-methyl-amino]pyrrolo[2,3-d]pyrimidine-7-carboxamide hydrochloride

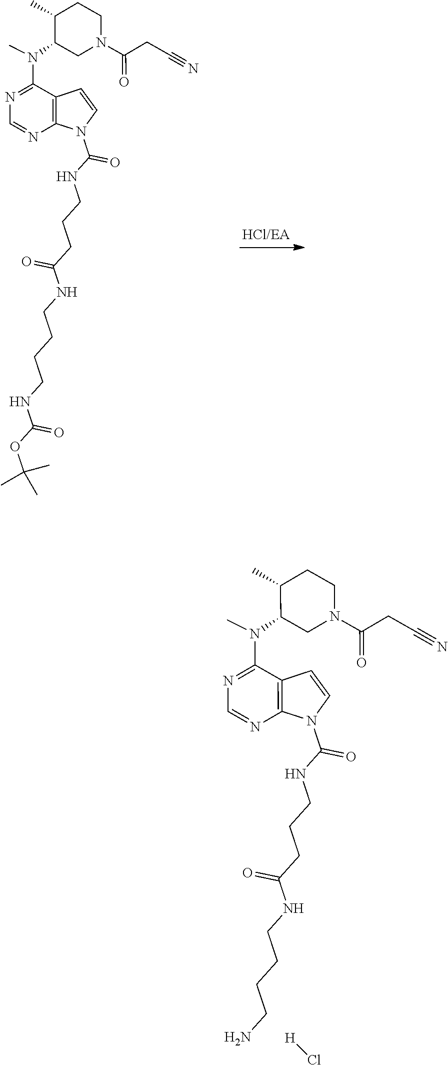

Following Step 2 of Example 1, tert-butyl N-[4-[4-[[4-[[(3R,4R)-1-(2-cyanoacetyl)-4-methyl-3-piperidyl]-methyl-amino]pyrrolo[2,3-d]pyrimidine-7-carbonyl]amino]butanoylamino]butyl]carbamate (0.5 g, 0.818 mmol) afford the title compound as a white solid (0.32 g, yield: 83%). MS (m/z): [MH 1-1]+ calcd for C₂₅H₃₇N₉O₃, 512.30; found, 512.3.

### Step 3: Preparation of conjugate of N-[4-(4-aminobutylamino)-4-oxo-butyl]-4-[[(3R,4R)-1-(2-cyanoacetyl)-4-methyl-3-piperidyl]-methyl-amino]pyrrolo[2,3-d]pyrimidine-7-carboxamide and HA

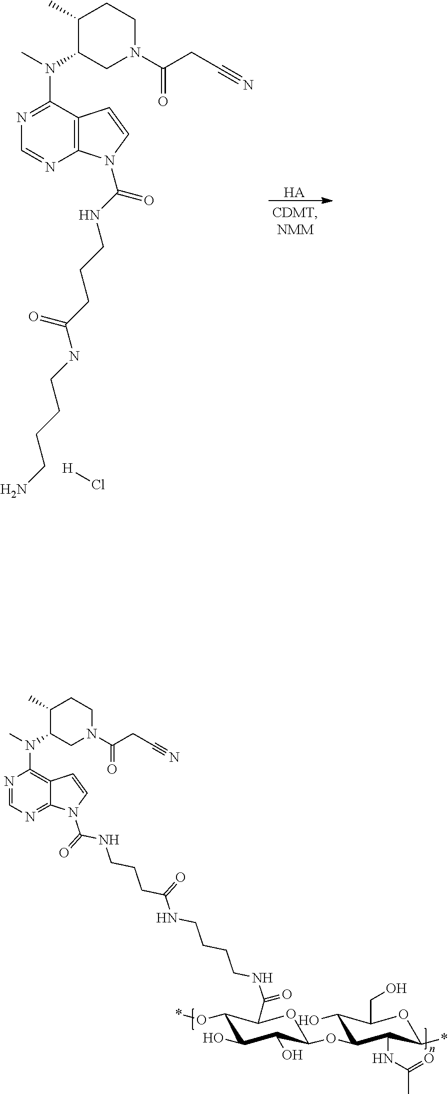

Following Step 3 of Example 1, sodium hyaluronate (MW 50 KDa, 147.2 mg, 0.365 mmol) and N-[4-(4-aminobutylamino)-4-oxo-butyl]-4-[[(3R,4R)-1-(2-cyanoacetyl)-4-methyl-3-piperidyl]-methyl-amino]pyrrolo[2,3-d]pyrimidine-7-carboxamide hydrochloride (200 mg, 0.365 mmol) afforded the title compound 0.162 g, Yield: 47.5%, DSR: 33%; ¹H NMR (400 MHz, D₂O) δ ppm 8.26-7.86 (m Hz, 0.33H), 7.64-7.18 (m, 0.33H), 6.81-6.32 (m, 0.33H), 4.52-4.28 (m, 2.33H), 4.06-2.81 (m, 14.95H), 2.41-2.21 (m, 0.99H), 1.99-1.63 (m, 4.32H), 1.47-1.26 (m, 1.32H), 1.11-0.91 (m, 0.99H).

With Step 3 of Example 1, reaction of sodium hyaluronate (MW 500 KDa) provided corresponding product (0.243 g, Yield: 71.3%, DSR: 18%),

With Step 3 of Example 1, reaction of sodium hyaluronate (MW 2000 KDa) provided corresponding product (0.168 g, Yield: 49.3%, DSR: 20%).

### Example 41

### Preparation of conjugate of N-[4-(4-aminobutylcarbamoyl)phenyl]-4-[[(3R,4R)-1-(2-cyanoacetyl)-4-methyl-3-piperidyl]-methyl-amino]pyrrolo[2,3-d]pyrimidine-7-carboxamide and HA

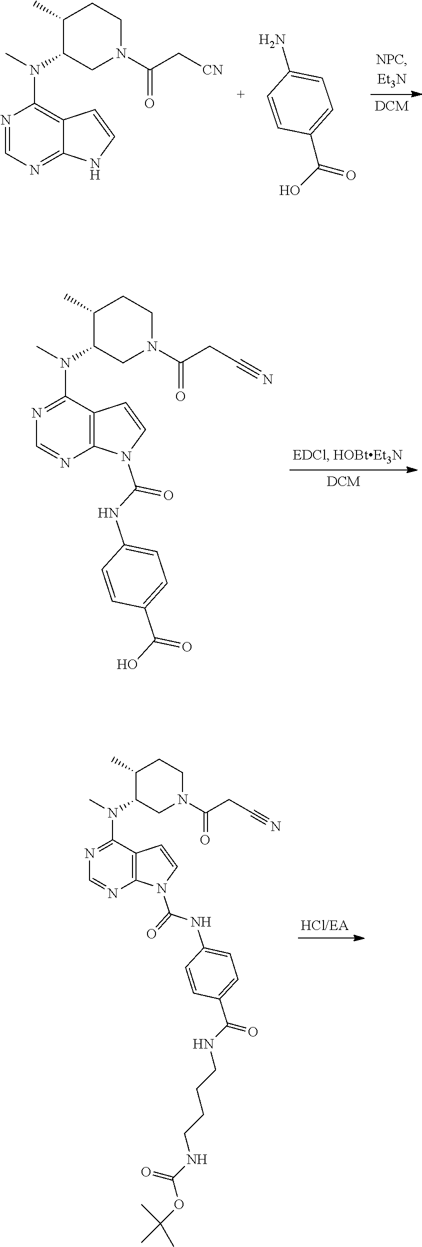 [SMILES: C[C@@H]1CCN(C(=O)CC#N)C[C@@H]1N(C)c1ncnc2[nH]ccc12 ; Nc1ccc(C(=O)O)cc1 ; C[C@@H]1CCN(C(=O)CC#N)C[C@@H]1N(C)c1ncnc2c1ccn2C(=O)Nc1ccc(C(=O)O)cc1 ; C[C@@H]1CCN(C(=O)CC#N)C[C@@H]1N(C)c1ncnc2c1ccn2C(=O)Nc1ccc(C(=O)NCCCCNC(=O)OC(C)(C)C)cc1]

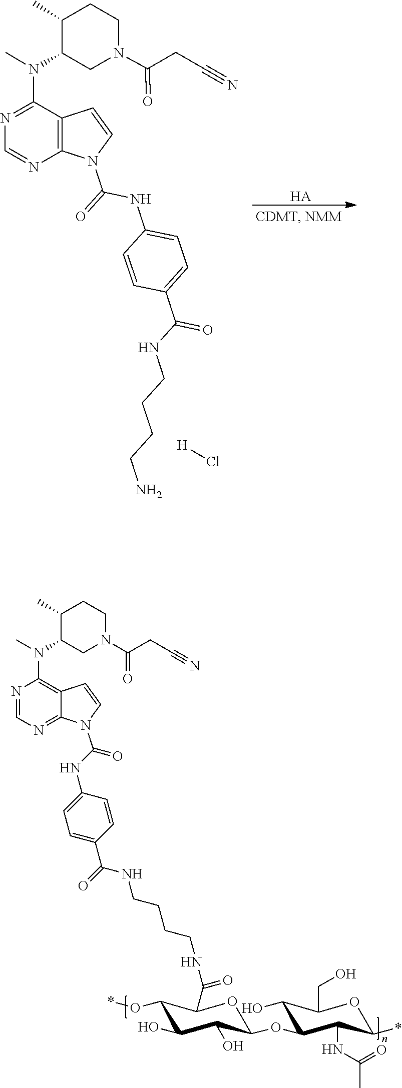

### Step 1: Preparation of 4-[[4-[[(3R,4R)-1-(2-cyanoacetyl)-4-methyl-3-piperidyl]-methyl-amino]pyrrolo[2,3-d]pyrimidine-7-carbonyl]amino]benzoic acid

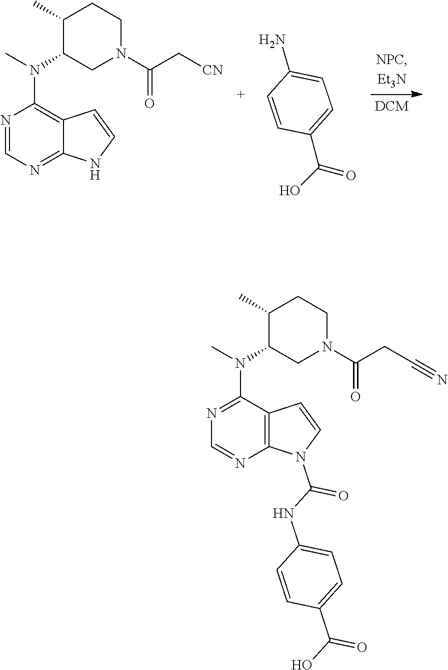 [SMILES: C[C@@H]1CCN(C(=O)CC#N)C[C@@H]1N(C)c1ncnc2[nH]ccc12 ; Nc1ccc(C(=O)O)cc1 ; C[C@@H]1CCN(C(=O)CC#N)C[C@@H]1N(C)c1ncnc2c1ccn2C(=O)Nc1ccc(C(=O)O)cc1]

Following Step 1 of Example 1, tofacitinib (10 g, 32 mmol) and 4-Aminobenzoic acid (8.7 g, 64 mmol) afforded the title compound (9.5 g, yield: 62.5%). MS (m/z): [M+] calcd for C₂₄H₂₅N₇O₄, 476.1; found, 476.3.

### Step 2: Preparation of tert-butyl N-[4-[[4-[[4-[[(3R,4R)-1-(2-cyanoacetyl)-4-methyl-3-piperidyl]-methyl-amino]pyrrolo[2,3-d]pyrimidine-7-carbonyl]amino]benzoyl]amino]butyl]carbamate

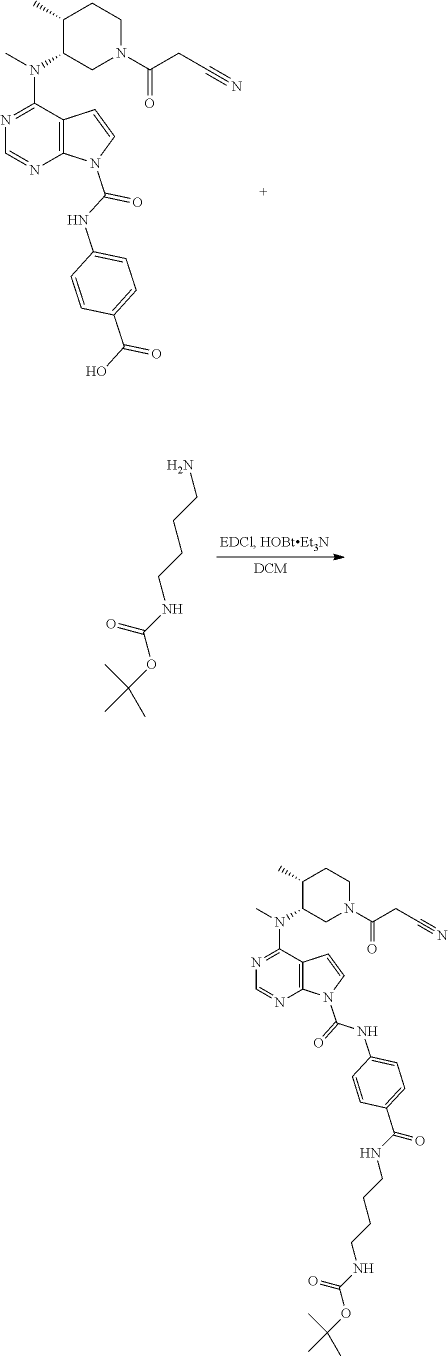 [SMILES: C[C@@H]1CCN(C(=O)CC#N)C[C@@H]1N(C)c1ncnc2c1ccn2C(=O)Nc1ccc(C(=O)O)cc1 ; CC(C)(C)OC(=O)NCCCCN ; C[C@@H]1CCN(C(=O)CC#N)C[C@@H]1N(C)c1ncnc2c1ccn2C(=O)Nc1ccc(C(=O)NCCCCNC(=O)OC(C)(C)C)cc1]

4-[[4-[[(3R,4R)-1-(2-cyanoacetyl)-4-methyl-3-piperidyl]-methyl-amino]pyrrolo[2,3-d]pyrimidine-7-carbonyl]amino]benzoic acid (950 mg, 2 mmol), N-Boc-1,4-butane diamine (564 mg, 3 mmol), 1-ethyl-3-(3-dimethylaminopropyl)carbodiimide (EDCI, 575.1 mg, 3 mmol) and 1-hydroxybenzotriazole (HOBt, 405 mg, 3 mmol) were dissolved in dichloromethane (24 mL) and triethylamine (0.84 mL, 6 mmol) was added. The reaction mixture was stirred at room temperature for 16 h. The solution was diluted with dichloromethane and washed with water and saturated brine solution. The organic layer was dried over sodium sulfate, filtered and then concentrated under reduced pressure. The crude residue was purified by column chromatography (ethylacetate:Hexane=10:100-50:100) to afford the title compound (0.89 g, Yield: 69%). MS (m/z): [M+H]+ calcd for C₃₃H₄₃N₉O₅, 646.33; found, 646.1. ¹H NMR (400 MHz, d-DMSO) δ ppm 12.21 (s, 1H), 8.42 (s, 2H), 7.89 (d, J=7.6 Hz, 2H), 7.73 (d, J=7.8 Hz, 3H), 6.84 (d, J=55.3 Hz, 2H), 4.89 (s, 1H), 4.18-3.60 (m, 4H), 3.47-3.32 (m, 2H), 3.23 (s, 5H), 2.92 (s, 2H), 2.42-2.33 (m, 1H), 1.90-1.17 (m, 15H), 1.01 (d, J=5.2 Hz, 3H).

### Step 3: Preparation of N-[4-(4-aminobutylcarbamoyl)phenyl]-4-[[(3R,4R)-1-(2-cyanoacetyl)-4-methyl-3-piperidyl]-methyl-amino]pyrrolo[2,3-d]pyrimidine-7-carboxamide hydrochloride

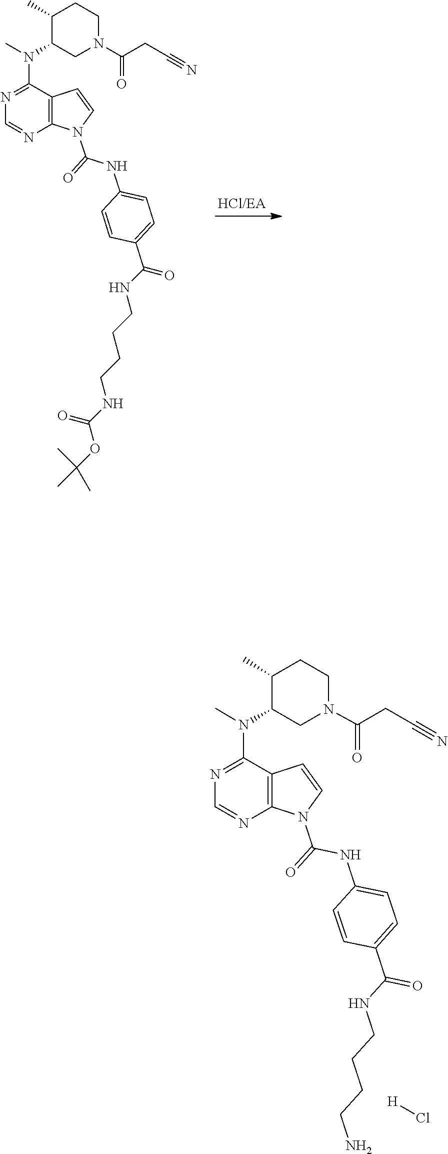

Following Step 2 of Example 1, tert-butyl N-[4-[[4-[[4-[[(3R,4R)-1-(2-cyanoacetyl)-4-methyl-3-piperidyl]-methyl-amino]pyrrolo[2,3-d]pyrimidine-7-carbonyl]amino]benzoyl]amino]butyl]carbamate (0.87 g, 1.35 mmol) afforded the title compound as a white solid (1 g, Yield: 83%). MS (m/z): [M+H]+ calcd for C₂₈H₃₅N₉O₃, 546.28; found, 546.3.

### Step 4 Preparation of conjugate of N-[4-(4-aminobutylcarbamoyl)phenyl]-4-[[(3R,4R)-1-(2-cyanoacetyl)-4-methyl-3-piperidyl]-methyl-amino]pyrrolo[2,3-d]pyrimidine-7-carboxamide and HA

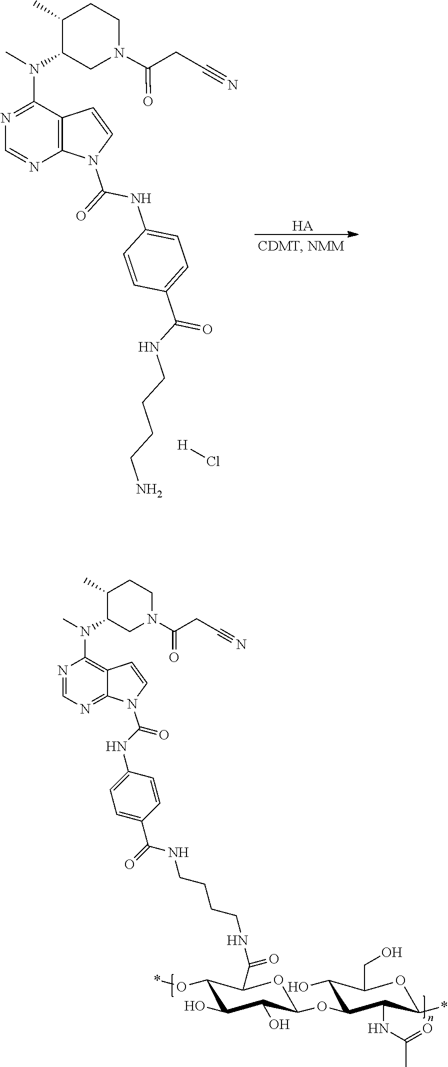

Following Step 3 of Example 1, sodium hyaluronate (MW 50 KDa, 138.6 mg, 0.344 mmol) and N-[4-(4-aminobutylcarbamoyl)phenyl]-4-[[(3R,4R)-1-(2-cyanoacetyl)-4-methyl-3-piperidyl]-methyl-amino]pyrrolo[2,3-d]pyrimidine-7-carboxamide hydrochloride (200 mg, 0.344 mmol) afforded the title compound 0.26 g, Yield: 93.9%, DSR: 23%; ¹H NMR (400 MHz, D₂O) δ ppm 8.32-8.12 (m, 0.23H), 7.88-7.23 (m, 1.15H), 6.80-6.64 (m, 0.23H), 4.54-4.30 (m, 2.23H), 3.99-2.81 (m, 12.99H), 2.06-1.37 (m, 4.61H), 1.04-0.87 (m, 0.69H).

With Step 3 of Example 1, reaction of sodium hyaluronate (MW 500 KDa) provided corresponding product (0.145 g, Yield: 52.4%, DSR: 15%),

With Step 3 of Example 1, reaction of sodium hyaluronate (MW 2000 KDa) provided corresponding product (0.104 g, Yield: 37.5%, DSR: 33%).

### Example 42

### Preparation of conjugate of N-[2-(4-aminobutylamino)-2-oxo-ethyl]-4-[[(3R,4R)-1-(2-cyanoacetyl)-4-methyl-3-piperidyl]-methyl-amino]pyrrolo[2,3-d]pyrimidine-7-carboxamide and HA

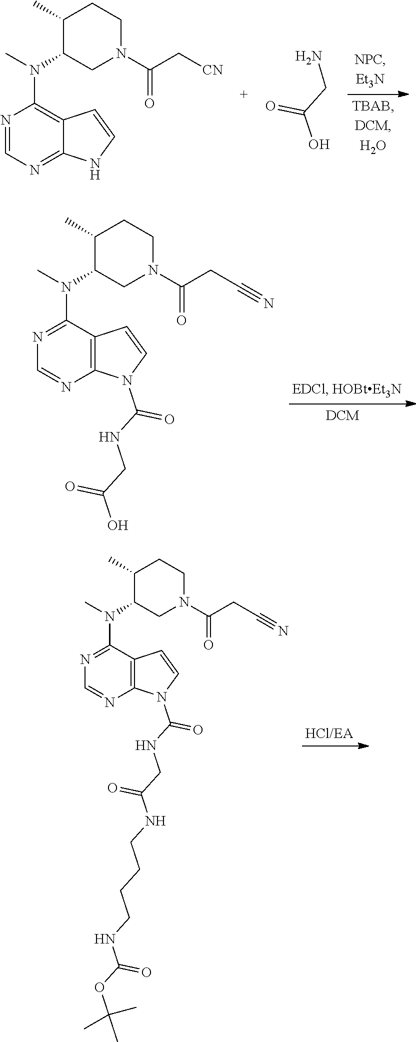

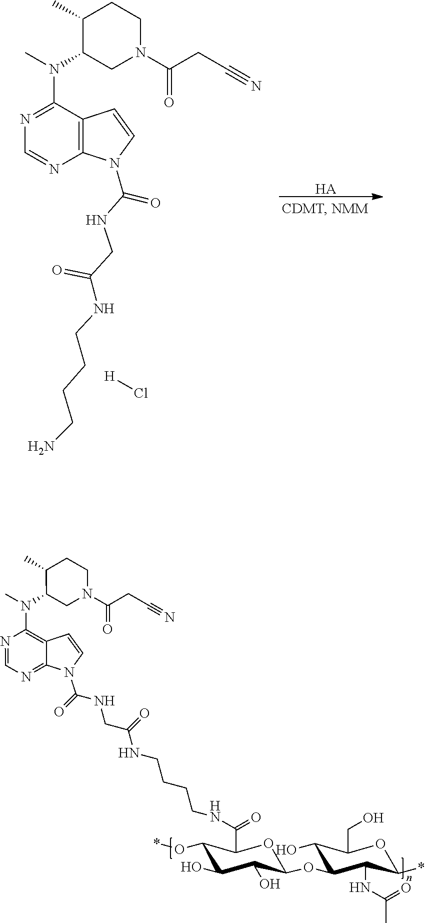

### Step 1: Preparation of 2-[[4-[[(3R,4R)-1-(2-cyanoacetyl)-4-methyl-3-piperidyl]-methyl-amino]pyrrolo[2,3-d]pyrimidine-7-carbonyl]amino]acetic acid

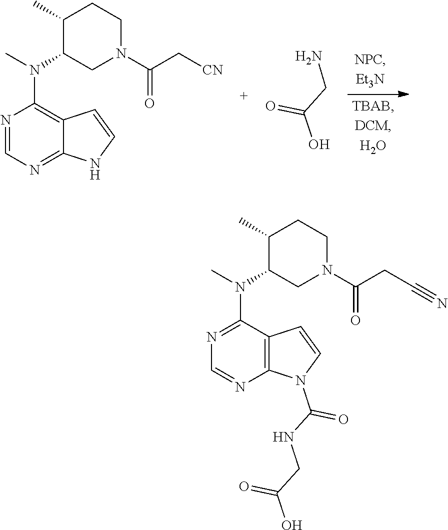 [SMILES: C[C@@H]1CCN(C(=O)CC#N)C[C@@H]1N(C)c1ncnc2[nH]ccc12 ; NCC(=O)O ; C[C@@H]1CCN(C(=O)CC#N)C[C@@H]1N(C)c1ncnc2c1ccn2C(=O)NCC(=O)O]

To a stirred mixture of tofacitinib (100 g, 320.1 mmol) and bis(4-nitrophenyl) carbonate (146 g, 480.1 mmol) in dichloromethane (2000 mL) was added triethylamine (97.2 g, 960.3 mmol). The reaction mixture was heated to 45° C. and stirred at this temperature for 16 h. Then the reaction mixture was cooled to 0-10° C. 300 mL aqueous solution of Glycine (48.1 g, 640.2 mmol), triethylamine (32 g, 316.8 mmol) and tetrabutylammonium bromide (10.3 g) were added. The reaction mixture was stirred at 0˜10° C. for 16 h, the pH value of the reaction mixture was adjusted to 3-4 with acetic acid and the organic layer was washed with 0.5N hydrochloric acid solution, water and saturated brine solution. The organic layer was dried over sodium sulfate, filtered and then concentrated under reduced pressure. The crude residue was purified by column chromatography (methanol/dichloromethane=1:50˜1:10) to afford the title compound (89.8 g, Yield: 67.8%). MS (m/z): [M+H]+ calcd for C₁₉H₂₃N₇O₄, 414.1; found, 414.5.

### Step 2: Preparation of tert-butyl N-[4-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]butyl]carbamate

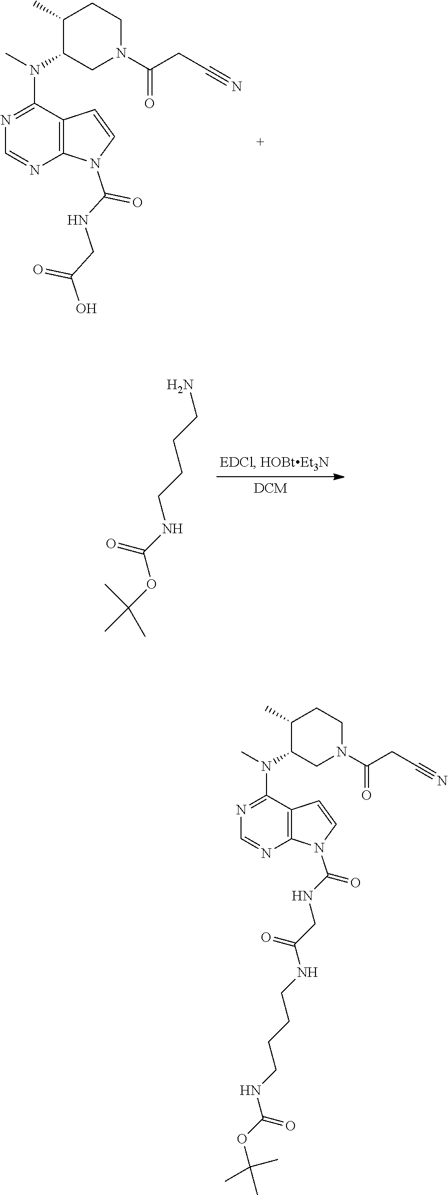 [SMILES: C[C@@H]1CCN(C(=O)CC#N)C[C@@H]1N(C)c1ncnc2c1ccn2C(=O)NCC(=O)O ; CC(C)(C)OC(=O)NCCCCN ; C[C@@H]1CCN(C(=O)CC#N)C[C@@H]1N(C)c1ncnc2c1ccn2C(=O)NCC(=O)NCCCCNC(=O)OC(C)(C)C]

2-[[4-[[(3R,4R)-1-(2-cyanoacetyl)-4-methyl-3-piperidyl]-methyl-amino]pyrrolo[2,3-d]pyrimidine-7-carbonyl]amino]acetic acid (619.5 mg, 1.5 mmol), N-Boc-1,4-butanediamine (423 mg, 2.25 mmol), 1-ethyl-3(3-dimethylamino propyl)carbodiimide (EDCI, 431.3 mg, 2.25 mmol) and 1-hydroxybenzotriazole (HOBt, 303.7 mg, 2.25 mmol) were dissolved in dichloromethane (10 mL) and triethylamine (0.62 mL, 4.5 mmol) was added. The reaction mixture was stirred at room temperature for 16 h. The solution was diluted with dichloromethane and washed with water and saturated brine solution. The organic layer was dried over sodium sulfate, filtered and then concentrated under reduced pressure. The crude residue was purified by column chromatography (methanol/dichloromethane=1:100-1:40) to afford the title compound (0.65 g, Yield: 74%). MS (m/z): [M+H]+ calcd for C₂₈H₄₁N₉O₅, 584.32; found, 584.2. ¹H NMR (400 MHz, d-DMSO) δ ppm 9.92 (t, J=5.2 Hz, 1H), 8.32 (d, J=5.8 Hz, 1H), 8.07 (t, J=5.4 Hz, 1H), 7.63 (d, J=4.0 Hz, 1H), 6.79 (d, J=26.3 Hz, 2H), 4.86 (s, 1H), 4.16-3.89 (m, 4H), 3.86-3.54 (m, 2H), 3.43-3.33 (m, 2H), 3.28 (s, 3H), 3.03 (t, J=19.0 Hz, 2H), 2.88 (d, J=5.7 Hz, 2H), 2.38 (s, 1H), 1.72 (dt, J=78.7, 27.2 Hz, 3H), 1.45-1.18 (m, 12H), 1.00 (d, J=7.1 Hz, 3H).

### Step 3: Preparation of N-[2-(4-aminobutylamino)-2-oxo-ethyl]-4-[[(3R,4R)-1-(2-cyanoacetyl)-4-methyl-3-piperidyl]-methyl-amino]pyrrolo[2,3-d]pyrimidine-7-carboxamide hydrochloride

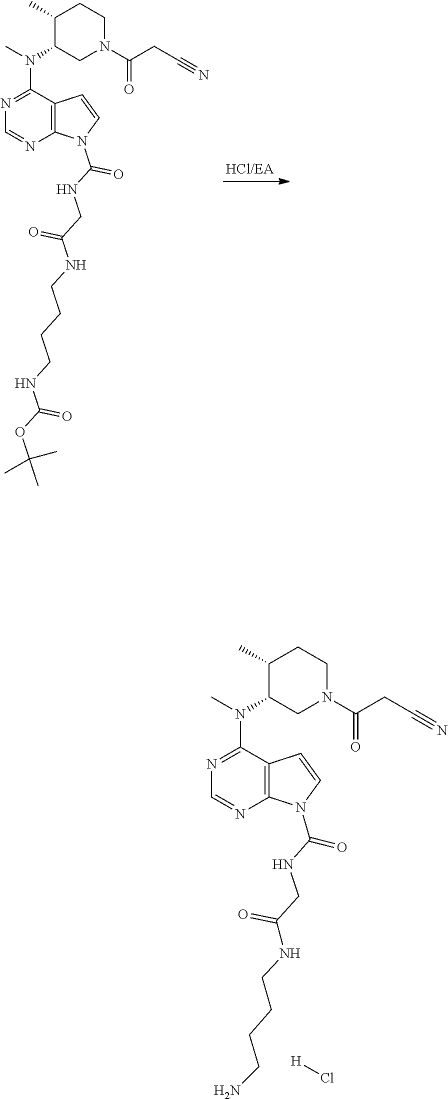

Following Step 2 of Example 1, tert-butyl N-[4-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]butyl]carbamate (0.3 g, 0.51 mmol) afforded the title compound as a white solid (0.26 g, Yield: 98%). MS (m/z): [M+H]+ calcd for C₂₃H₃₃N₉O₃, 484.27; found, 484.3. ¹H NMR (400 MHz, d-DMSO) δ ppm 9.90 (s, 1H), 8.34 (d, J=6.7 Hz, 1H), 8.21 (s, 1H), 7.91 (s, 2H), 7.66 (s, 1H), 6.84 (s, 1H), 4.85 (s, 1H), 4.13-3.66 (m, 7H), 3.39 (s, 1H), 3.28 (s, 1H), 3.10 (d, J=5.3 Hz, 2H), 2.70 (d, J=37.4 Hz, 2H), 2.34 (d, J=27.1 Hz, 2H), 1.77 (d, J=41.2 Hz, 1H), 1.55-1.39 (m, 4H), 1.15 (t, J=7.1 Hz, 2H), 1.00 (d, J=6.5 Hz, 3H).

### Step 4: Preparation of conjugate of N-[2-(4-aminobutylamino)-2-oxo-ethyl]-4-[[(3R,4R)-1-(2-cyanoacetyl)-4-methyl-3-piperidyl]-methyl-amino]pyrrolo[2,3-d]pyrimidine-7-carboxamide and HA

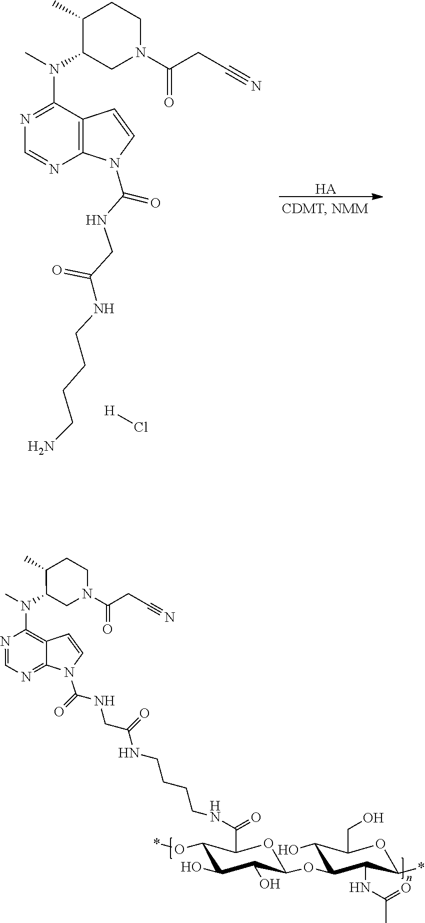

Following Step 3 of Example 1, sodium hyaluronate (MW 50 KDa, 101 mg, 0.25 mmol) and N-[2-(4-aminobutylamino)-2-oxo-ethyl]-4-[[(3R,4R)-1-(2-cyanoacetyl)-4-methyl-3-piperidyl]-methyl-amino]pyrrolo[2,3-d]pyrimidine-7-carboxamide hydrochloride (130 mg, 0.25 mmol) afforded the title compound 0.102 g, Yield: 45%, DSR: 40%; ¹H NMR (400 MHz, D₂O) δ ppm 8.26-7.85 (m, 0.4H), 7.63-7.18 (m, 0.4H), 6.83-6.31 (m, 0.4H), 4.58-4.28 (m, 2.4H), 4.15-2.90 (m, 16H), 2.43-2.10 (m, 0.8H), 2.06-1.69 (m, 4.4H), 1.59-1.37 (m, 1.2H), 1.19-0.87 (m, 0.8H).

With Step 3 of Example 1, reaction of sodium hyaluronate (MW 500 KDa) provided corresponding product (0.092 g, Yield: 40.6%, DSR: 26%),

With Step 3 of Example 1, reaction of sodium hyaluronate (MW 2000 KDa) provided corresponding product (0.143 g, Yield: 63%, DSR: 21%).

### Example 43

### Preparation of conjugate of N-[2-(2-aminoethylamino)-2-oxo-ethyl]-4-[[(3R,4R)-1-(2-cyanoacetyl)-4-methyl-3-piperidyl]-methyl-amino]pyrrolo[2,3-d]pyrimidine-7-carboxamide and HA

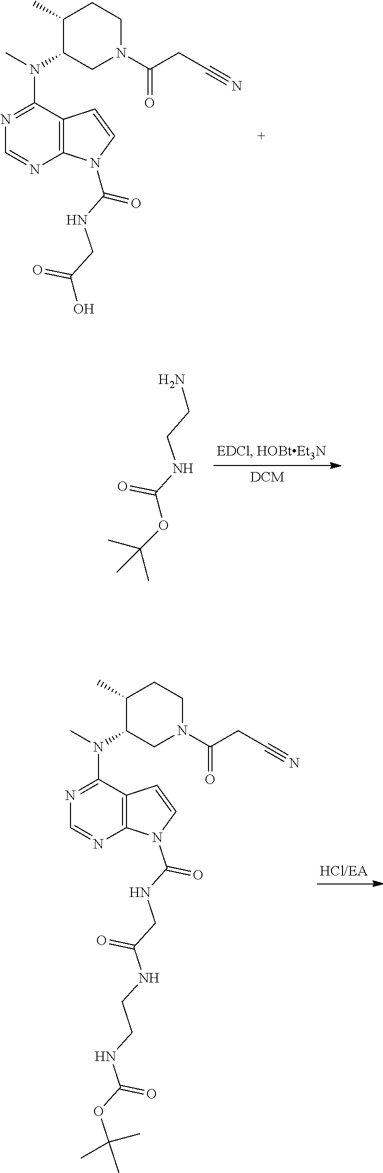 [SMILES: C[C@@H]1CCN(C(=O)CC#N)C[C@@H]1N(C)c1ncnc2c1ccn2C(=O)NCC(=O)O ; CC(C)(C)OC(=O)NCCN ; C[C@@H]1CCN(C(=O)CC#N)C[C@@H]1N(C)c1ncnc2c1ccn2C(=O)NCC(=O)NCCNC(=O)OC(C)(C)C]

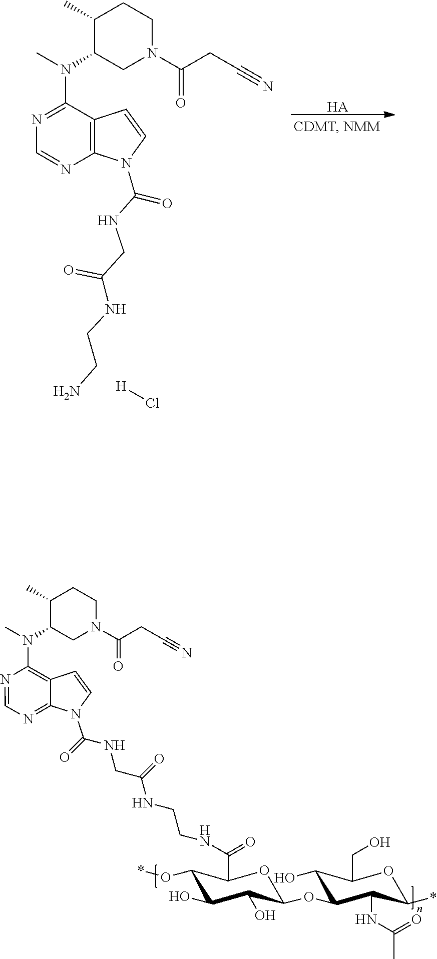

### Step 1: Preparation of tert-butyl N-[2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]ethyl]carbamate

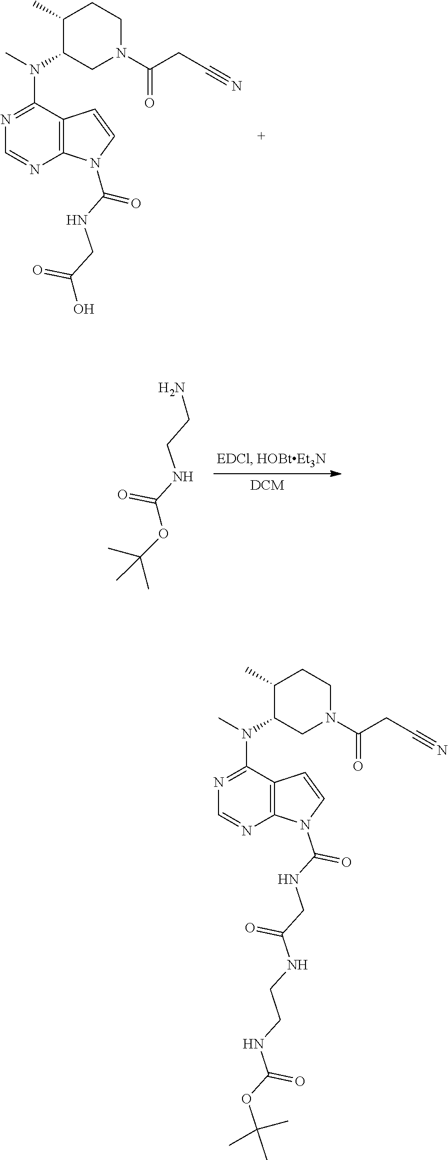 [SMILES: C[C@@H]1CCN(C(=O)CC#N)C[C@@H]1N(C)c1ncnc2c1ccn2C(=O)NCC(=O)O ; CC(C)(C)OC(=O)NCCN ; C[C@@H]1CCN(C(=O)CC#N)C[C@@H]1N(C)c1ncnc2c1ccn2C(=O)NCC(=O)NCCNC(=O)OC(C)(C)C]

2-[[4-[[(3R,4R)-1-(2-cyanoacetyl)-4-methyl-3-piperidyl]-methyl-amino]pyrrolo[2,3-d]pyrimidine-7-carbonyl]amino]acetic acid (1239 mg, 3 mmol), N-Boc-ethylenediamine (721 mg, 4.5 mmol), 1-ethyl-3-(3-dimethylamino propyl)carbodiimide (EDCI, 863 mg, 4.5 mmol) and 1-hydroxybenzotriazole (HOBt, 607.5 mg, 4.5 mmol) were dissolved in dichloromethane (40 mL) and triethylamine (1.25 mL, 9 mmol) was added. The reaction mixture was stirred at room temperature for 16 h. The solution was diluted with dichloromethane and washed with water and saturated brine solution. The organic layer was dried over sodium sulfate, filtered and then concentrated under reduced pressure. The crude residue was purified by column chromatography (Methanol/dichloromethane=1:100˜1:60) to afford the title compound (1.1 g, Yield: 66%). MS (m/z): [M+FH]+ calcd for C₂₆H₃₇N₉O₅, 556.29; found, 556.2. ¹H NMR (400 MHz, CDCl₃) δ ppm 10.37-10.14 (m, 1H), 8.31 (d, J=6.3 Hz, 1H), 7.76-7.54 (m, 1H), 7.03 (t, J=18.4 Hz, 1H), 6.59 (d, J=3.9 Hz, 1H), 5.13 (s, 1H), 5.01 (s, 1H), 4.26-4.11 (m, 2H), 4.09-3.72 (m, 2H), 3.67-3.46 (m, 4H), 3.42-3.33 (m, 5H), 3.28 (dd, J=10.8, 5.5 Hz, 2H), 2.50 (ddd, J=18.5, 12.3, 6.0 Hz, 1H), 2.09-1.84 (m, 1H), 1.83-1.72 (m, 1H), 1.54-1.30 (m, 9H), 1.09 (dd, J=11.8, 7.1 Hz, 3H).

### Step 2: Preparation of N-[2-(2-aminoethylamino)-2-oxo-ethyl]-4-[[(3R,4R)-1-(2-cyanoacetyl)-4-methyl-3-piperidyl]-methyl-amino]pyrrolo[2,3-d]pyrimidine-7-carboxamide hydrochloride

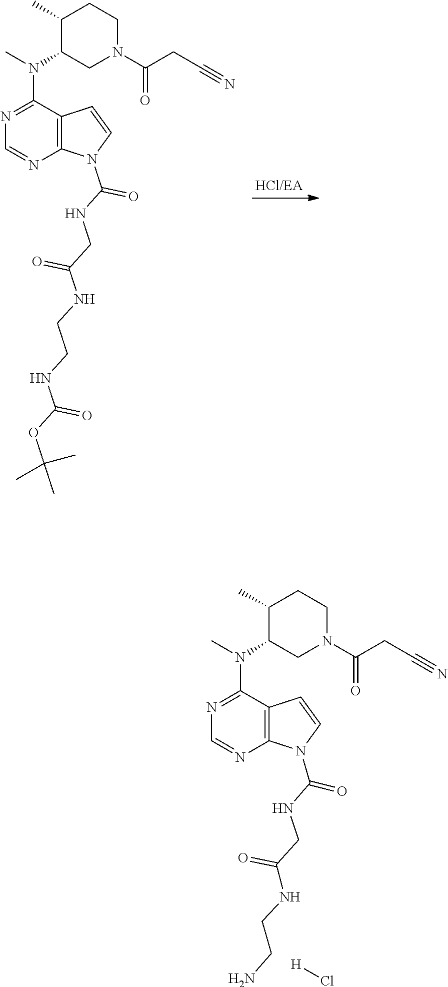

Following Step 2 of Example 1, tert-butyl N-[2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]ethyl]carbamate (0.89 g, 1.6 mmol) afforded the title compound as a white solid (0.78 g, Yield: 99%). MS (m/z): [M+H]+ calcd for C₂₁H₂₉N₉O₃, 456.23; found, 456.2.

### Step 3: Preparation of conjugate of N-[2-(2-aminoethylamino)-2-oxo-ethyl]-4-[[(3R,4R)-1-(2-cyanoacetyl)-4-methyl-3-piperidyl]-methyl-amino]pyrrolo[2,3-d]pyrimidine-7-carboxamide and HA

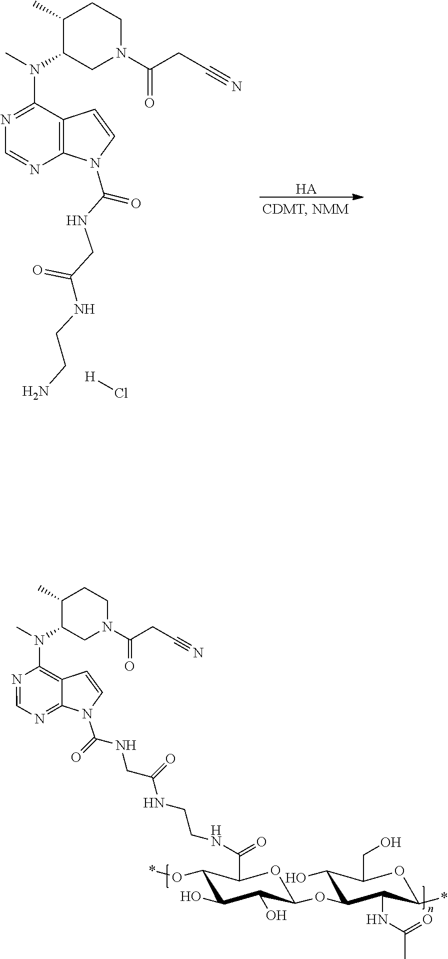

Following Step 3 of Example 1, sodium hyaluronate (MW 50 KDa, 152.7 mg, 0.379 mmol) and N-[2-(2-aminoethylamino)-2-oxo-ethyl]-4-[[(3R,4R)-1-(2-cyanoacetyl)-4-methyl-3-piperidyl]-methyl-amino]pyrrolo[2,3-d]pyrimidine-7-carboxamide hydrochloride (200 mg, 0.379 mmol) afforded the title compound 0.165 g, Yield: 47.7%, DSR: 40%; ¹H NMR (400 MHz, D₂O) δ ppm 8.26-7.82 (m, 0.4H), 7.60-7.26 (m, 0.4H), 6.77-6.26 (m, 0.4H), 4.60-4.21 (m, 2.4H), 4.08-2.99 (m, 16H), 2.42-2.18 (m, 0.4H), 2.04-1.55 (m, 3.8H), 1.06-0.85 (m, 1.2H).

With Step 3 of Example 1, reaction of sodium hyaluronate (MW 500 KDa) provided corresponding product (0.173 g, Yield: 50%, DSR: 28%),

With Step 3 of Example 1, reaction of sodium hyaluronate (MW 2000 KDa) provided corresponding product (0.18 g, Yield: 52%, DSR: 40%).

### Example 44

### Preparation of conjugate of N-[2-[4-(2-aminoethyl)anilino]-2-oxo-ethyl]-4-[[(3R,4R)-1-(2-cyanoacetyl)-4-methyl-3-piperidyl]-methyl-amino]pyrrolo[2,3-d]pyrimidine-7-carboxamide and HA

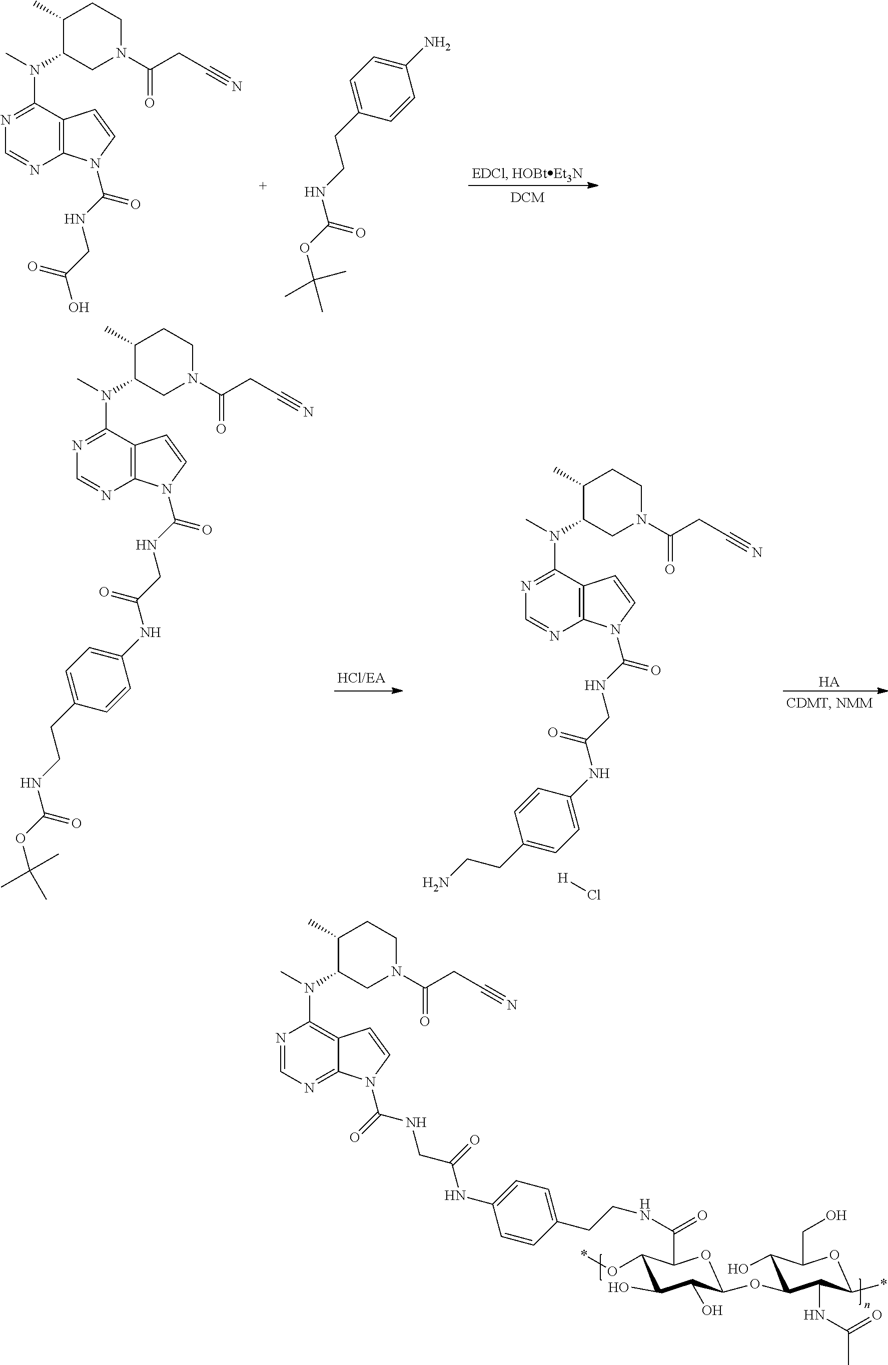

### Step 1: Preparation of tert-butyl N-[2-[4-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]phenyl]ethyl]carbamate

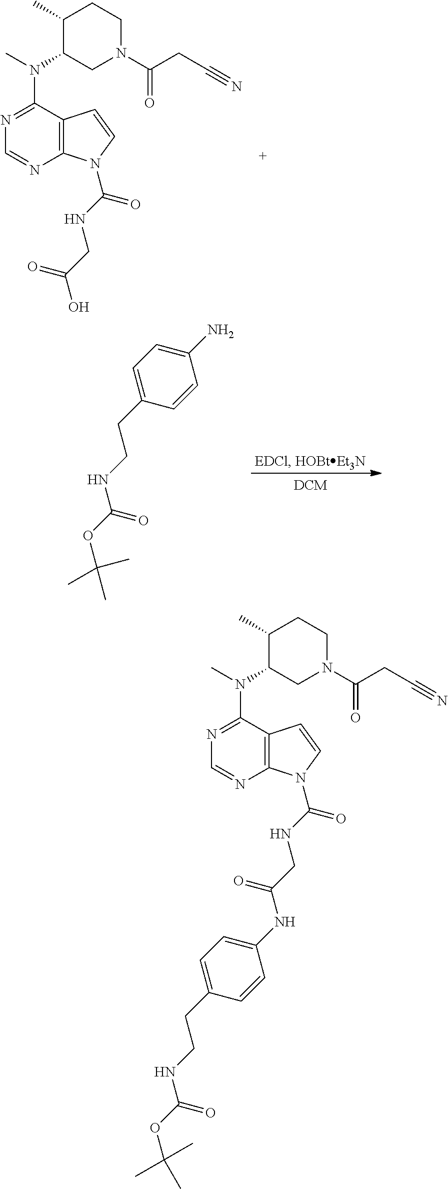 [SMILES: C[C@@H]1CCN(C(=O)CC#N)C[C@@H]1N(C)c1ncnc2c1ccn2C(=O)NCC(=O)O ; CC(C)(C)OC(=O)NCCc1ccc(N)cc1 ; C[C@@H]1CCN(C(=O)CC#N)C[C@@H]1N(C)c1ncnc2c1ccn2C(=O)NCC(=O)Nc1ccc(CCNC(=O)OC(C)(C)C)cc1]

2-[[4-[[(3R,4R)-1-(2-cyanoacetyl)-4-methyl-3-piperidyl]-methyl-amino]pyrrolo[2,3-d]pyrimidine-7-carbonyl]amino]acetic acid (1652 mg, 4 mmol), tert-Butyl 4-aminophenethylcarbamate (1418 mg, 6 mmol), 1-ethyl-3-(3-dimethylaminopropyl)carbodiimide (EDCI, 1151 mg, 6 mmol) and 1-hydroxybenzotriazole (HOBt, 810 mg, 6 mmol) were dissolved in dichloromethane (40 mL) and triethylamine (1.665 mL, 12 mmol) was added. The reaction mixture was stirred at room temperature for 16 b. The solution was diluted with dichloromethane and washed with water and saturated brine solution. The organic layer was dried over sodium sulfate, filtered and then concentrated under reduced pressure. The crude residue was purified by column chromatography (Methanol/dichloromethane=1:150˜1:100) to afford the title compound (1.77 g, Yield: 70%). MS (m/z): [M+H]+ calcd for C₃₂H₄₁N₉O₅, 632.32; found, 632.2. ¹H NMR (400 MHz, CDCl₃) δ ppm 10.59-10.19 (m, 1H), 8.30 (dd, J=17.1, 7.1 Hz, 2H), 7.67 (dd, J=13.2, 4.0 Hz, 1H), 7.49 (t, J=11.3 Hz, 2H), 7.13 (d, J=8.2 Hz, 2H), 6.61 (t, J=4.7 Hz, 1H), 5.13 (s, 1H), 4.54 (s, 1H), 4.38-4.23 (m, 2H), 4.13-3.72 (m, 2H), 3.68-3.44 (m, 4H), 3.42-3.24 (m, 5H), 2.75 (t, J=6.8 Hz, 2H), 2.50 (ddd, J=19.1, 11.9, 5.6 Hz, 1H), 2.08-1.83 (m, 1H), 1.83-1.64 (m, 1H), 1.43 (s, 9H), 1.09 (t, J=8.2 Hz, 3H).

### Step 2: Preparation of N-[2-[4-(2-aminoethyl)anilino]-2-oxo-ethyl]-4-[[(3R,4R)-1-(2-cyanoacetyl)-4-methyl-3-piperidyl]-methyl-amino]pyrrolo[2,3-d]pyrimidine-7-carboxamide hydrochloride

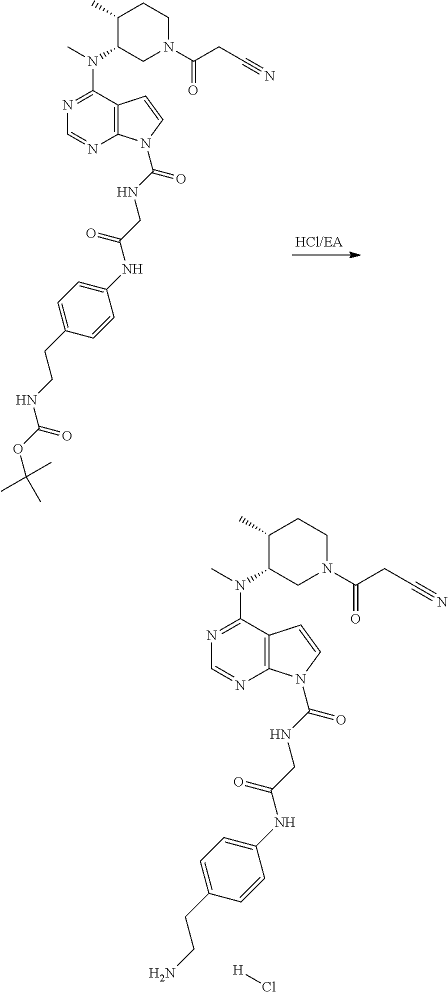 [SMILES: C[C@@H]1CCN(C(=O)CC#N)C[C@@H]1N(C)c1ncnc2c1ccn2C(=O)NCC(=O)Nc1ccc(CCNC(=O)OC(C)(C)C)cc1 ; C[C@@H]1CCN(C(=O)CC#N)C[C@@H]1N(C)c1ncnc2c1ccn2C(=O)NCC(=O)Nc1ccc(CCN)cc1 ; Cl]

Following Step 2 of Example 1, tert-butyl N-[2-[4-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]phenyl]ethyl]carbamate (1.48 g, 2.345 mmol) afforded the title compound as a white solid (1.3 g, Yield: 97.7%). MS (m/z): [M+H]+ calcd for C₂₇H₃₃N₉O₃, 532.27; found, 532.2.

### Step 3: Preparation of conjugate of N-[2-[4-(2-aminoethyl)anilino]-2-oxo-ethyl]-4-[[(3R,4R)-1-(2-cyanoacetyl)-4-methyl-3-piperidyl]-methyl-amino]pyrrolo[2,3-d]pyrimidine-7-carboxamide and HA

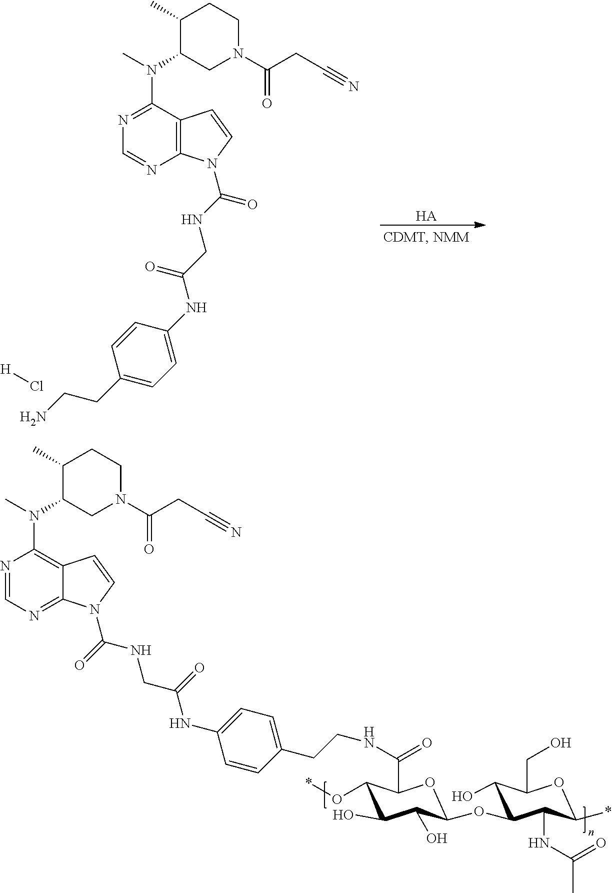

Following Step 3 of Example 1, sodium hyaluronate (MW 50 KDa, 133.4 mg, 0331 mmol) and N-[2-[4-(2-aminoethyl)anilino]-2-oxo-ethyl]-4-[[(3R,4R)-1-(2-cyanoacetyl)-4-methyl-3-piperidyl]-methyl-amino]pyrrolo[2,3-d]pyrimidine-7-carboxamide hydrochloride (200 mg, 0.331 mmol) afforded the title compound 0.136 g, Yield: 41.5%, DSR: 20%; ¹H NMR (400 MHz, D₂O) δ ppm 8.33-8.06 (m, 0.2H), 7.68-7.50 (m, 0.2H), 7.48-7.13 (m, 0.8H), 6.85-6.67 (m, 0.2H), 4.59-4.23 (m, 2.2H), 4.07-2.84 (m, 12.8H), 2.46-2.22 (m, 0.4H), 2.05-1.70 (m, 3.2H), 1.21-1.25 (mz, 0.2H), 1.07-0.90 (m, 0.6H).

With Step 3 of Example 1, reaction of sodium hyaluronate (MW 500 KDa) provided corresponding product (0.158 g, Yield: 48.2%, DSR: 22%),

With Step 3 of Example 1, reaction of sodium hyaluronate (MW 2000 KDa) provided corresponding product (0.146 g, Yield: 44.5%, DSR: 16%).

### Example 45

### Preparation of conjugate of methyl (2S)-6-amino-2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]hexanoate and HA

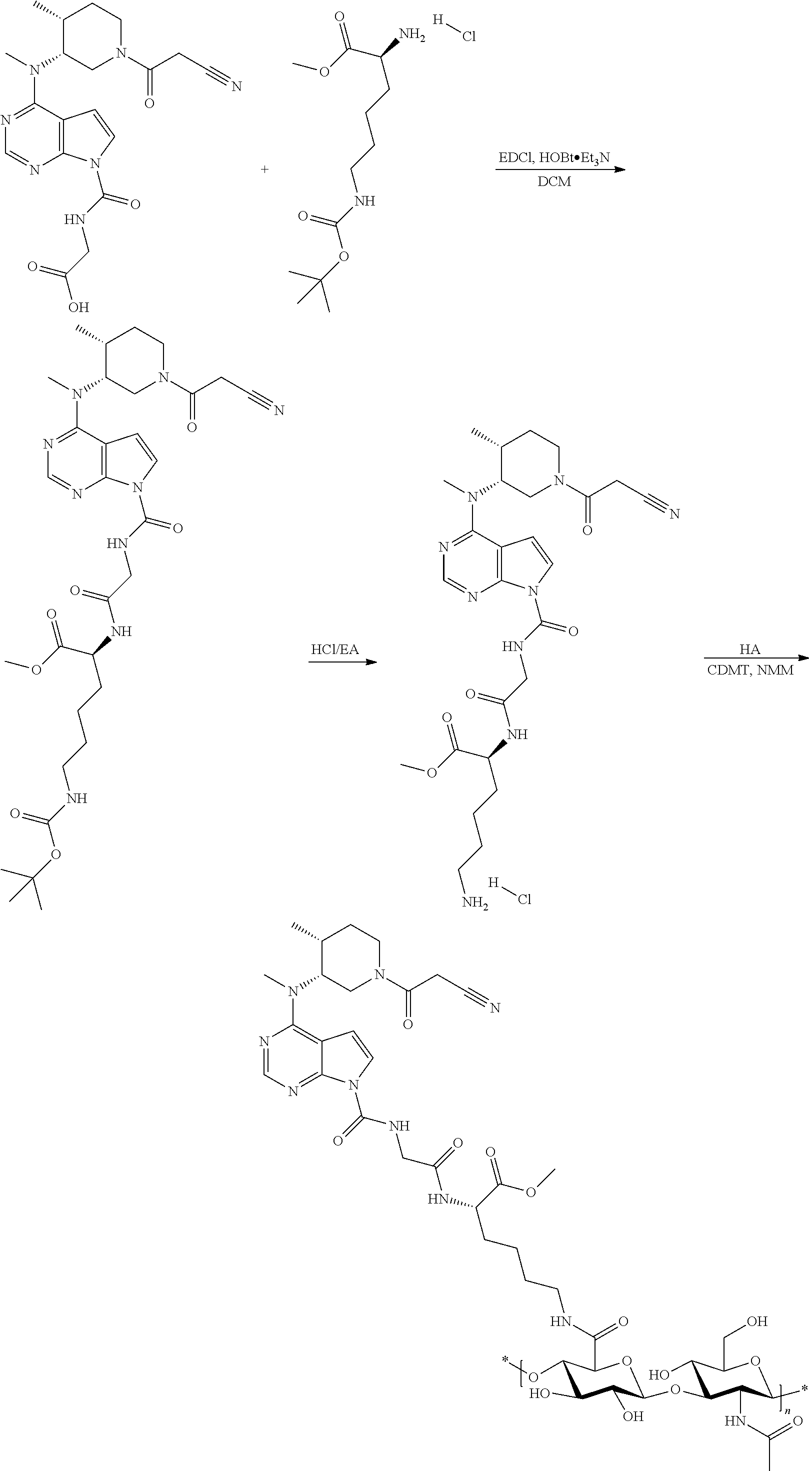

### Step 1: Preparation of methyl (2S)-6-(tert-butoxycarbonylamino)-2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]hexanoate

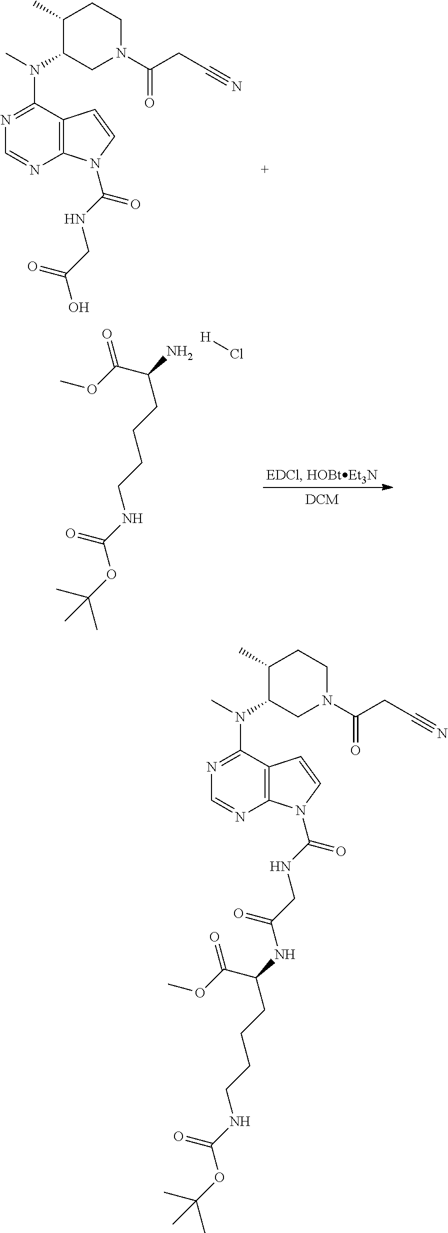

2-[[4-[[(3R,4R)-1-(2-cyanoacetyl)-4-methyl-3-piperidyl]-methyl-amino]pyrrolo[2,3-d]pyrimidine-7-carbonyl]amino]acetic acid (1239 mg, 3 mmol), (S)-methyl 6-amino-2-((tert-butoxycarbonyl)-amino)hexanoate (1336 mg, 4.5 mmol), 1-ethyl-3-(3-dimethylaminopropyl)carbodiimide (EDCI, 863 mg, 4.5 mmol) and 1-hydroxybenzotriazole (HOBt, 608 mg, 4.5 mmol) were dissolved in dichloromethane (40 mL) and triethylamine (2.1 mL, 15 mmol) was added. The reaction mixture was stirred at room temperature for 16 h. The solution was diluted with dichloromethane and washed with water and saturated brine solution. The organic layer was dried over sodium sulfate, filtered and then concentrated under reduced pressure. The crude residue was purified by column chromatography (methanol/dichloromethane=1:150-1:80) to afford the title compound (1.638 g, Yield: 83.3%). MS (n/z): [M+H]+ calcd for C₃₁H₄₅N₉O₇, 656.34; found, 656.1. ¹H NMR (400 MHz, CDCl₃) δ ppm 10.36 (dd, J=12.6, 7.1 Hz, 1H), 8.33 (d, J=9.1 Hz, 1H), 7.72 (dd, J=12.9, 4.0 Hz, 1H), 6.72 (d, J=7.8 Hz, 1H), 6.61 (t, J=4.2 Hz, 1H), 5.14 (s, 1H), 4.63 (dt, J=33.6, 16.8 Hz, 2H), 4.19 (t, J=14.2 Hz, 2H), 4.11-3.76 (m, 2H), 3.73 (s, 3H), 3.66-3.57 (m, 1H), 3.55-3.39 (m, 3H), 3.36 (s, 2H), 3.17-2.98 (m, 2H), 2.59-2.41 (m, 1H), 2.03-1.84 (m, 2H), 1.83-1.68 (m, 2H), 1.53-1.39 (m, 11H), 1.39-1.26 (m, 3H), 1.09 (dd, J=12.3, 7.1 Hz, 3H).

### Step 2: Preparation of methyl (2S)-6-amino-2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]hexanoate hydrochloride

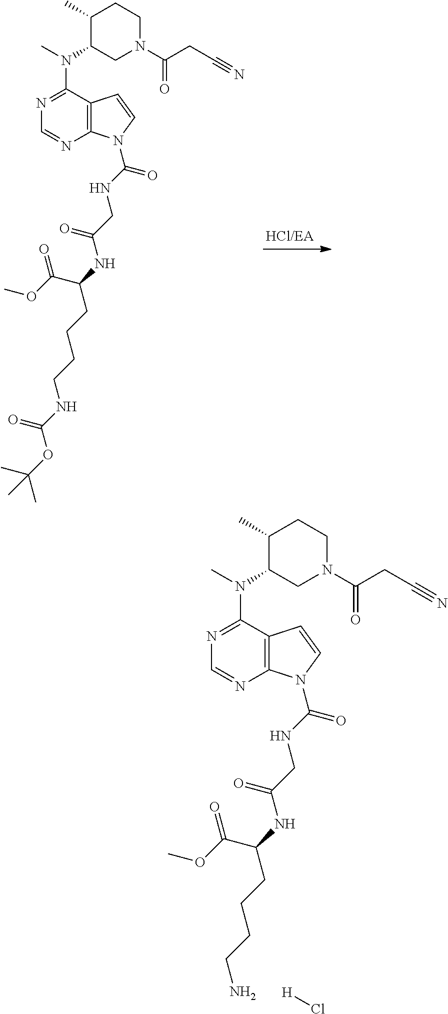 [SMILES: COC(=O)[C@H](CCCCNC(=O)OC(C)(C)C)NC(=O)CNC(=O)n1ccc2c(N(C)[C@H]3CN(C(=O)CC#N)CC[C@H]3C)ncnc21 ; COC(=O)[C@H](CCCCN)NC(=O)CNC(=O)n1ccc2c(N(C)[C@H]3CN(C(=O)CC#N)CC[C@H]3C)ncnc21 ; Cl]

Following Step 2 of Example 1, methyl (2S)-6-(tert-butoxycarbonylamino)-2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]hexanoate (1.4 g, 2.137 mmol) afforded the title compound as a white solid (1.26 g, Yield: 99.6%) that was used without further purification. MS (m/z): [M+H]+ calcd for C₂₆H₃₇N₉O₅, 556.29; found, 556.2.

### Step 3: Preparation of conjugate of methyl (2S)-6-amino-2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]hexanoate and HA

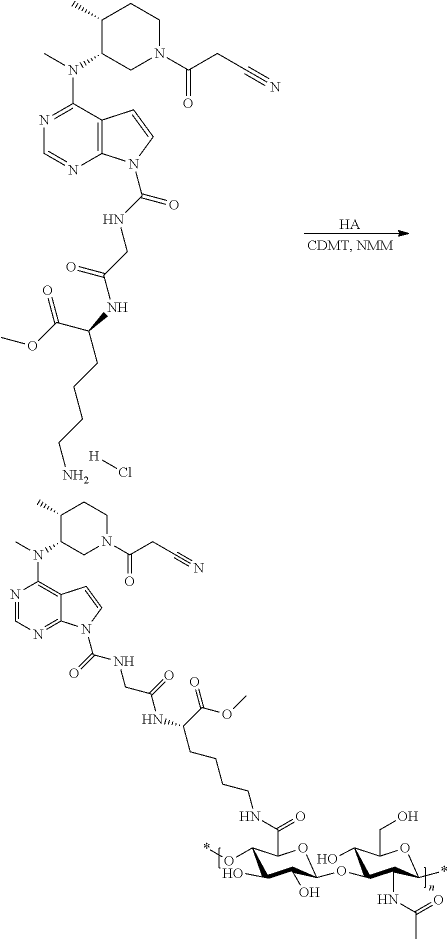

Following Step 3 of Example 1, sodium hyaluronate (MW 50 KDa, 128.2 mg, 0.318 mmol) and methyl(2S)-6-amino-2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]hexanoate hydrochloride (200 mg, 0.318 mmol) afforded the title compound 0.131 g, Yield: 40.6%, DSR: 30%; ¹H NMR (400 MHz, D₂O) δ ppm 8.27-7.99 (m, 0.3H), 7.64-7.33 (m, 0.3H), 6.82-6.51 (m, 0.3H), 4.58-4.30 (d, J=28.8 Hz, 2.3H), 4.10-2.99 (m, 14.5H), 2.41-2.27 (m, 0.3H), 2.09-1.58 (m, 4.8H), 1.53-1.29 (m, 0.9H), 1.25-0.93 (m, 0.9H).

With Step 3 of Example 1, reaction of sodium hyaluronate (MW 2000 KDa) provided corresponding product (0.134 g, Yield: 41.5%, DSR: 20%).

### Example 46

### Preparation of conjugate of N-[4-[4-(2-aminoethyl)anilino]-4-oxo-butyl]-4-[[(3R,4R)-1-(2-cyanoacetyl)-4-methyl-3-piperidyl]-methyl-amino]pyrrolo[2,3-d]pyrimidine-7-carboxamide and HA

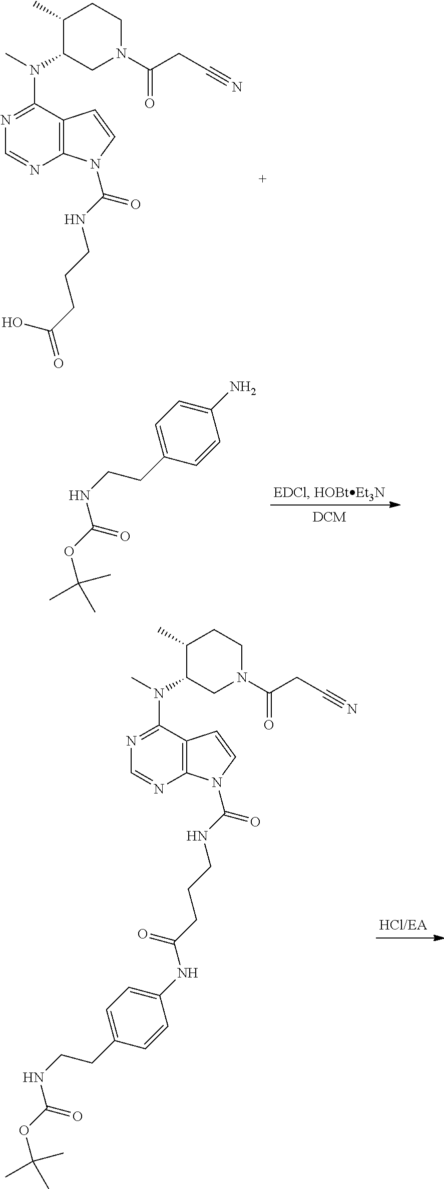 [SMILES: C[C@@H]1CCN(C(=O)CC#N)C[C@@H]1N(C)c1ncnc2c1ccn2C(=O)NCCCC(=O)O ; CC(C)(C)OC(=O)NCCc1ccc(N)cc1 ; C[C@@H]1CCN(C(=O)CC#N)C[C@@H]1N(C)c1ncnc2c1ccn2C(=O)NCCCC(=O)Nc1ccc(CCNC(=O)OC(C)(C)C)cc1]

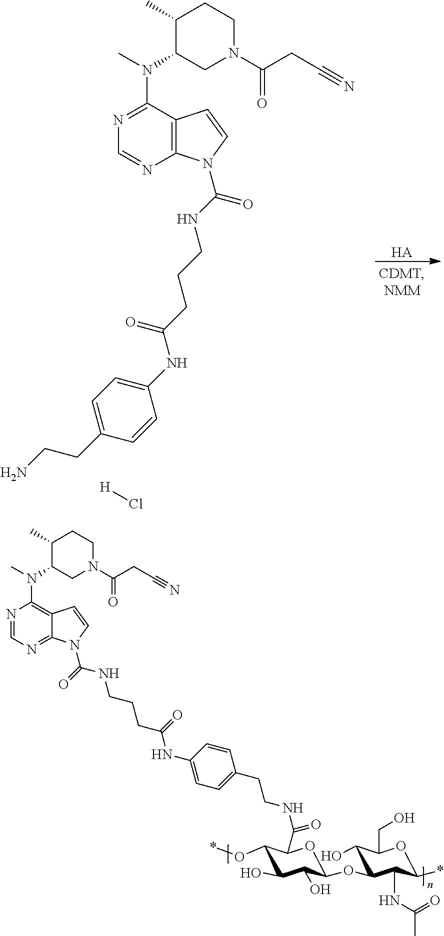

### Step 1: Preparation of tert-butyl N-[2-[4-[4-[[4-[[(3R,4R)-1-(2-cyanoacetyl)-4-methyl-3-piperidyl]-methyl-amino]pyrrolo[2,3-d]pyrimidine-7-carbonyl]amino]butanoylamino]phenyl]ethyl]carbamate

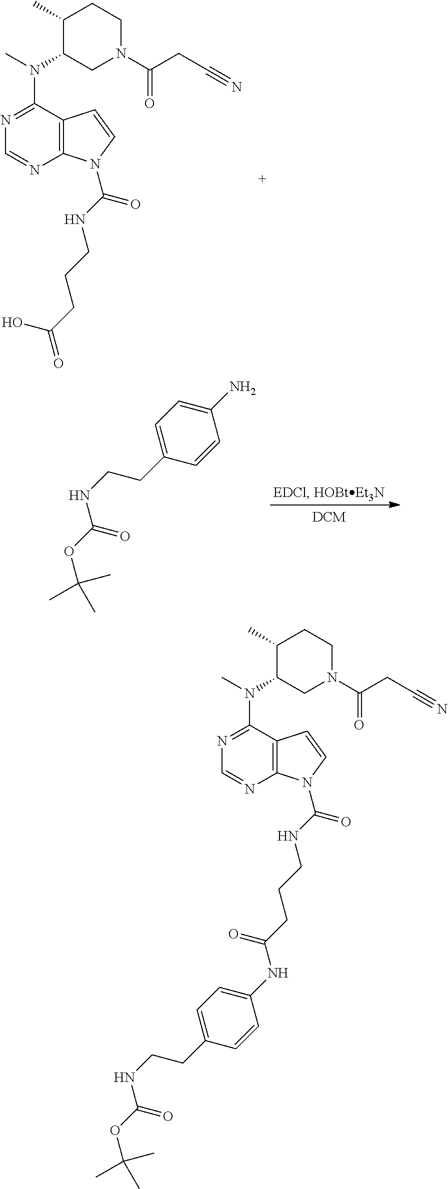 [SMILES: C[C@@H]1CCN(C(=O)CC#N)C[C@@H]1N(C)c1ncnc2c1ccn2C(=O)NCCCC(=O)O ; CC(C)(C)OC(=O)NCCc1ccc(N)cc1 ; C[C@@H]1CCN(C(=O)CC#N)C[C@@H]1N(C)c1ncnc2c1ccn2C(=O)NCCCC(=O)Nc1ccc(CCNC(=O)OC(C)(C)C)cc1]

4-[[4-[[(3R,4R)-1-(2-cyanoacetyl)-4-methyl-3-piperidyl]-methyl-amino]pyrrolo[2,3-d]pyrimidine-7-carbonyl]amino]butanoic acid (1764 mg, 4 mmol), tert-Butyl 4-aminophenethylcarbamate (1418 mg, 6 mmol), 1-ethyl-3-(3-dimethyl amino propyl)carbodiimide (EDCI, 1151 mg, 6 mmol) and 1-hydroxybenzotriazole (HOBt, 810 mg, 6 mmol) were dissolved in dichloromethane (40 mL) and triethylamine (1.66 mL, 12 mmol) was added. The reaction mixture was stirred at room temperature for 16 h. The solution was diluted with dichloromethane and washed with water and saturated brine solution. The organic layer was dried over sodium sulfate, filtered and then concentrated under reduced pressure. The crude residue was purified by column chromatography (methanol/dichloromethane=1:150˜1:100) to afford the title compound (2.02 g, Yield: 76.6%). MS (m/z): [M+H]+ calcd for C₃₄H₄₅N₉O₅, 660.35; found, 660.3. ¹H NMR (400 MHz, CDCl₃) δ ppm 10.00 (dd, J=14.1, 8.0 Hz, 1H), 8.48 (d, J=26.6 Hz, 1H), 8.28 (d, J=9.5 Hz, 1H), 7.73 (dd, J=10.8, 4.0 Hz, 1H), 7.54 (d, J=8.3 Hz, 2H), 7.13 (d, J=8.1 Hz, 2H), 6.60 (t, J=4.6 Hz, 1H), 5.14 (s, 1H), 4.56 (s, 1H), 4.12-3.71 (m, 2H), 3.62 (t, J=9.0 Hz, 3H), 3.48 (s, 3H), 3.35 (d, J=8.6 Hz, 5H), 2.76 (t, J=6.8 Hz, 2H), 2.60-2.35 (m, 3H), 2.14-2.04 (m, 2H), 2.02-1.84 (m, 1H), 1.84-1.68 (m, 1H), 1.43 (s, 9H), 1.10 (dd, J=12.0, 7.1 Hz, 3H).

### Step 2: Preparation of N-[4-[4-(2-aminoethyl)anilino]-4-oxo-butyl]-4-[[(3R,4R)-1-(2-cyanoacetyl)-4-methyl-3-piperidyl]-methyl-amino]pyrrolo[2,3-d]pyrimidine-7-carboxamide hydrochloride

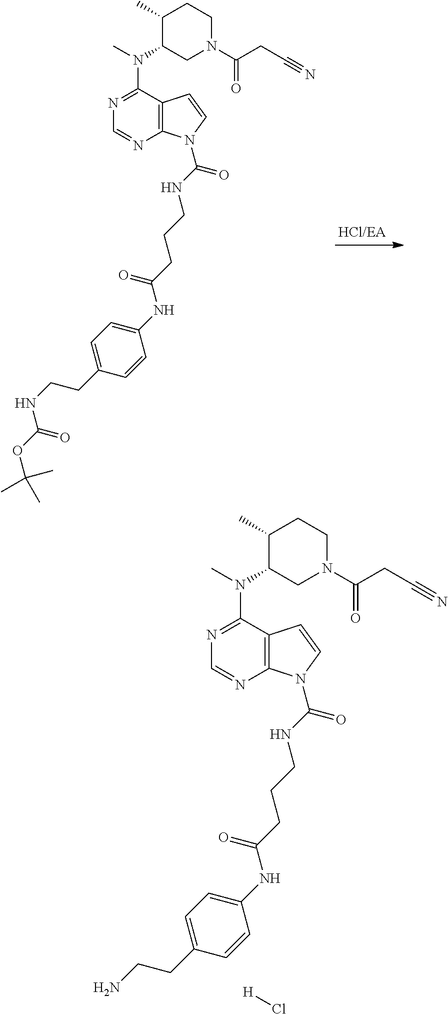 [SMILES: C[C@@H]1CCN(C(=O)CC#N)C[C@@H]1N(C)c1ncnc2c1ccn2C(=O)NCCCC(=O)Nc1ccc(CCNC(=O)OC(C)(C)C)cc1 ; C[C@@H]1CCN(C(=O)CC#N)C[C@@H]1N(C)c1ncnc2c1ccn2C(=O)NCCCC(=O)Nc1ccc(CCN)cc1 ; Cl]

Following Step 2 of Example 1, tert-butyl N-[2-[4-[4-[[4-[[(3R,4R)-1-(2-cyanoacetyl)-4-methyl-3-piperidyl]-methyl-amino]pyrrolo[2,3-d]pyrimidine-7-carbonyl]amino]butanoylamino]phenyl]ethyl]carbamate (1.73 g, 2.625 mmol) afforded the title compound as a white solid (1.56 g, Yield: 99.8%). MS (m/z): [M+H]+ calcd for C₂₉H₃₇N₉O₃, 560.30; found, 560.1.

### Step 3: Preparation of conjugate of N-[4-[4-(2-aminoethyl)anilino]-4-oxo-butyl]-4-[[(3R,4R)-1-(2-cyanoacetyl)-4-methyl-3-piperidyl]-methyl-amino]pyrrolo[2,3-d]pyrimidine-7-carboxamide and HA

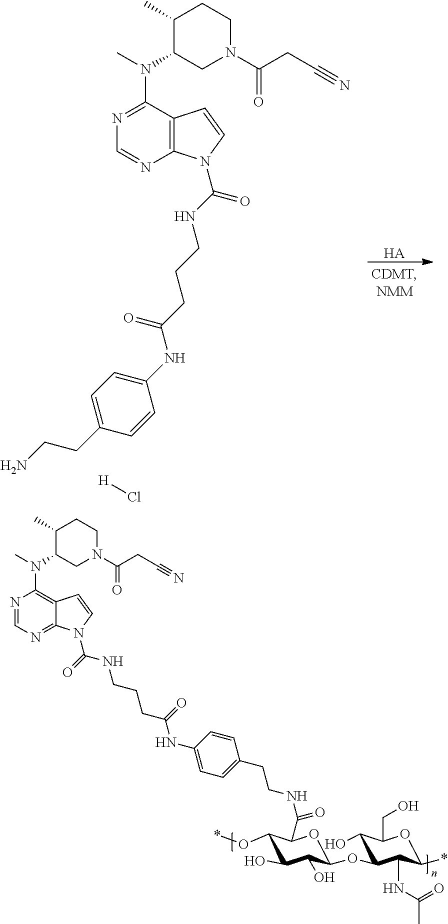

Following Step 3 of Example 1, sodium hyaluronate (MW 50 KDa, 127.3 mg, 0.316 mmol) and N-[4-[4-(2-aminoethyl)anilino]-4-oxo-butyl]-4-[[(3R,4R)-1-(2-cyanoacetyl)-4-methyl-3-piperidyl]-methyl-amino]pyrrolo[2,3-d]pyrimidine-7-carboxamidehydrochloride (200 mg, 0.316 mmol) afforded the title compound 0.154 g, Yield: 47.8%, DSR: 33%; ¹H NMR (400 MHz, D₂O) δ ppm 8.10-7.85 (m, 0.33H), 7.54-7.32 (m, 0.33H), 7.24-6.81 (m, 1.32H), 6.68-6.42 (m, 0.33H), 4.59-4.21 (m, 2.33H), 4.04-2.92 (m, 14.29H), 2.73-2.63 (m, 0.66H), 2.50-2.23 (m, 0.99H), 2.11-1.56 (m, 3.99H), 1.30-1.16 (m, 0.33H), 1.13-0.87 (m, 0.99H).

With Step 3 of Example 1, reaction of sodium hyaluronate (MW 500 KDa) provided corresponding product (0.136 g, Yield: 42.2%, DSR: 18%),

With Step 3 of Example 1, reaction of sodium hyaluronate (MW 2000 KDa) provided corresponding product (0.158 g, Yield: 49.1%, DSR: 17%).

### Example 47

### Preparation of conjugate of 4-[[(3R,4R)-1-(2-cyanoacetyl)-4-methyl-3-piperidylmethyl-amino]-N-[2-oxo-2-(4-piperazin-1-ylanilino)ethyl]pyrrolo[2,3-d]pyrimidine-7-carboxamide and HA

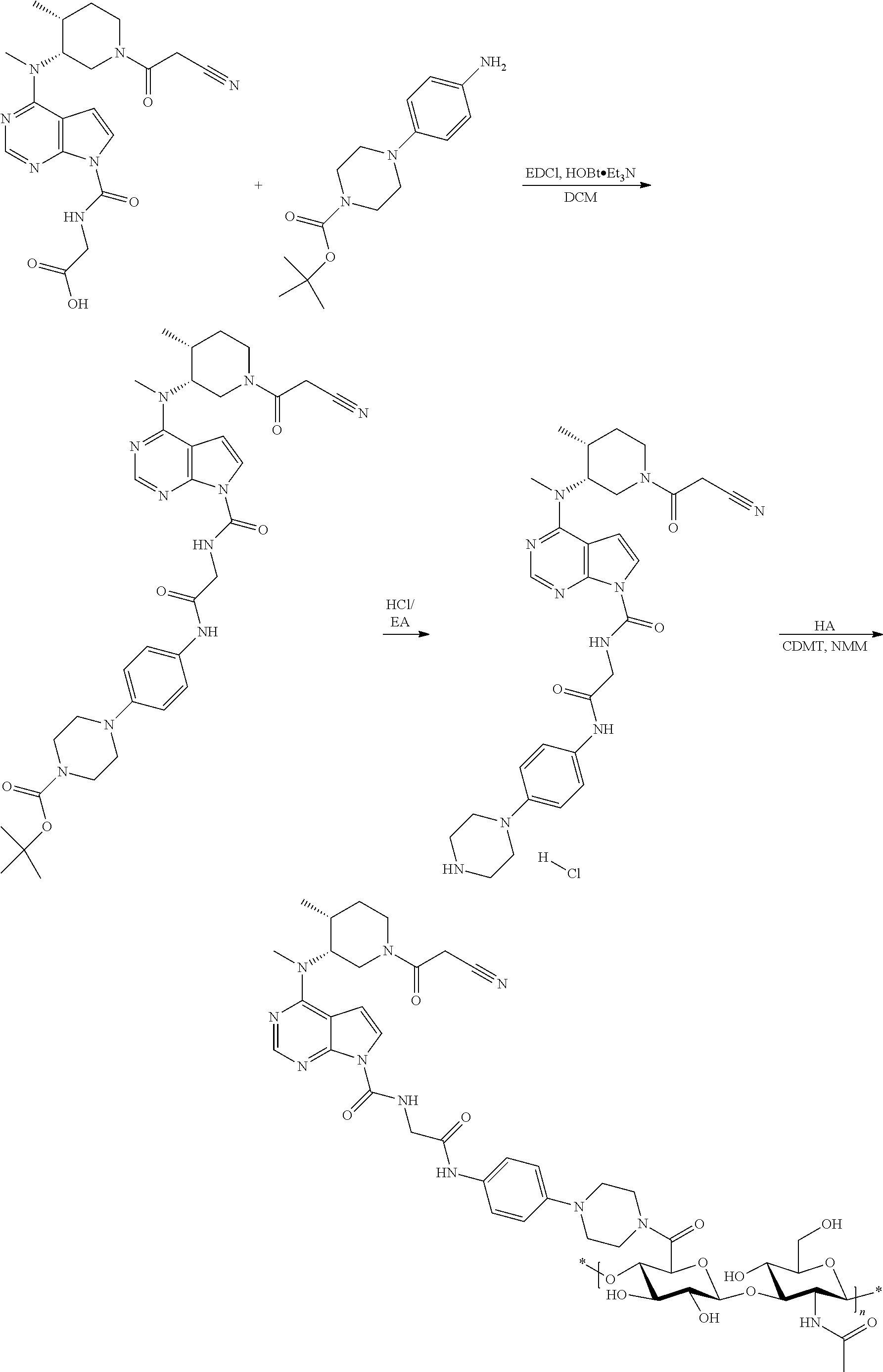

### Step 1: Preparation of tert-butyl 4-[4-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]phenyl]piperazine-1-carboxylate

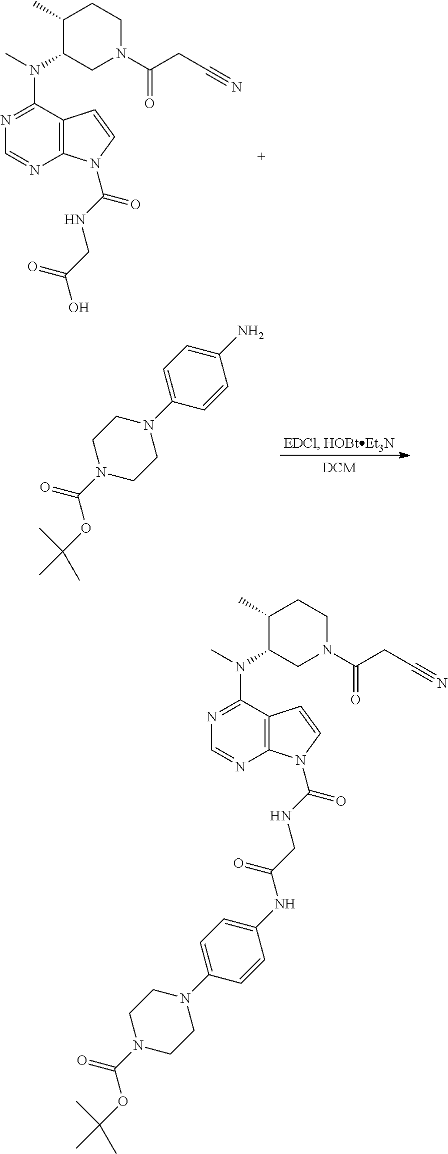 [SMILES: C[C@@H]1CCN(C(=O)CC#N)C[C@@H]1N(C)c1ncnc2c1ccn2C(=O)NCC(=O)O ; CC(C)(C)OC(=O)N1CCN(c2ccc(N)cc2)CC1 ; C[C@@H]1CCN(C(=O)CC#N)C[C@@H]1N(C)c1ncnc2c1ccn2C(=O)NCC(=O)Nc1ccc(N2CCN(C(=O)OC(C)(C)C)CC2)cc1]

2-[[4-[[(3R,4R)-1-(2-cyanoacetyl)-4-methyl-3-piperidyl]-methyl-amino]pyrrolo[2,3-d]pyrimidine-7-carbonyl]amino]acetic acid (619.5 mg, 1.5 mmol), tert-Butyl 4-(4-aminophenyl)piperazine-1-carboxylate (624 mg, 2.25 mmol), 1-ethyl-3-(3-dimethylaminopropyl)carbodiimide (EDCI, 431.3 mg, 2.25 mmol) and 1-hydroxybenzotriazole (HOBt, 304 mg, 2.25 mmol) were dissolved in dichloromethane (20 mL) and triethylamine (0.624 mL, 4.5 mmol) was added. The reaction mixture was stirred at room temperature for 16 h. The solution was diluted with dichloromethane and washed with water and saturated brine solution. The organic layer was dried over sodium sulfate, filtered and then concentrated under reduced pressure. The crude residue was purified by column chromatography (Methanol/dichloromethane=1:100-1:50) to afford the title compound (0.55 g, Yield: 54.5%). MS (m/z): [M+H]+ calcd for C₃₄H₄₄N₁₀O₅, 673.34; found, 673.3. MS (m/z): [M+H]+ calcd for C₃₄H₄₄N₁₀O₅, 673.34; found, 673.3. ¹H NMR (400 MHz, CDCl₃) δ ppm 10.45 (dd, J=13.6, 7.8 Hz, 1H), 8.33 (d, J=9.1 Hz, 1H), 8.07 (d, J=10.2 Hz, 1H), 7.69 (dd, J=12.1, 4.0 Hz, 1H), 7.43 (d, J=8.9 Hz, 2H), 6.88 (d, J=8.9 Hz, 2H), 6.62 (t, J=4.3 Hz, 1H), 5.14 (s, 1H), 4.30 (d, J=5.8 Hz, 2H), 4.11-3.74 (m, 2H), 3.68-3.43 (m, 8H), 3.38 (d, J=20.1 Hz, 3H), 3.10 (dd, J=28.1, 23.2 Hz, 4H), 2.60-2.39 (m, 1H), 2.01-1.84 (m, 1H), 1.83-1.66 (m, 1H), 1.48 (s, 9H), 1.09 (t, J=8.5 Hz, 3H).

### Step 2: Preparation of 4-[[(3R,4R)-1-(2-cyanoacetyl)-4-methyl-3-piperidyl]-methyl-amino]-N-[2-oxo-2-(4-piperazin-1-ylanilino)ethyl]pyrrolo[2,3-d]pyrimidine-7-carboxamide hydrochloride

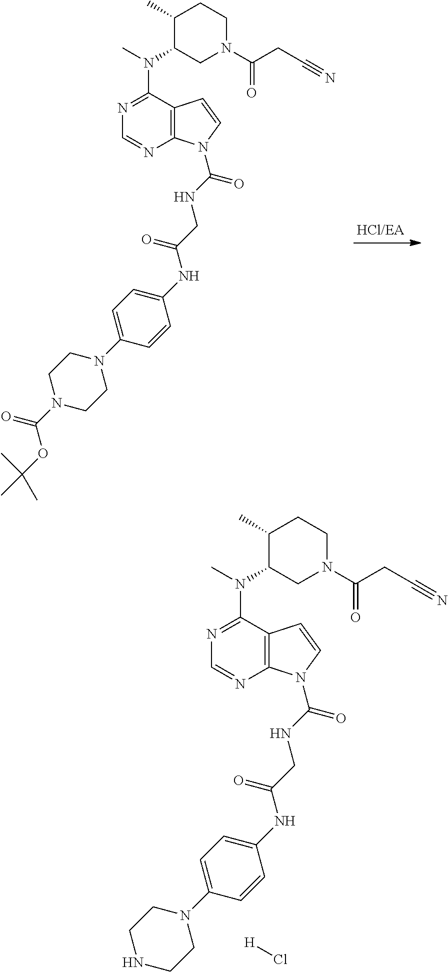 [SMILES: C[C@@H]1CCN(C(=O)CC#N)C[C@@H]1N(C)c1ncnc2c1ccn2C(=O)NCC(=O)Nc1ccc(N2CCN(C(=O)OC(C)(C)C)CC2)cc1 ; C[C@@H]1CCN(C(=O)CC#N)C[C@@H]1N(C)c1ncnc2c1ccn2C(=O)NCC(=O)Nc1ccc(N2CCNCC2)cc1 ; Cl]

Following Step 2 of Example 1, tert-butyl 4-[4-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]phenyl]piperazine-1-carboxylate (0.48 g, 0.714 mmol) afforded the title compound as a white solid (0.43 g, Yield: 99%). MS (m/z): [M+H]+ calcd for C₂₉H₃₆N₁₀O₃₁, 573.29; found, 573.2.

### Step 3: Preparation of conjugate of 4-[[(3R,4R)-1-(2-cyanoacetyl)-4-methyl-3-piperidyl methyl-amino]-N-[2-oxo-2-(4-piperazin-1-ylanilino)ethyl]pyrrolo [2,3-d]pyrimidine-7-carboxamide and HA

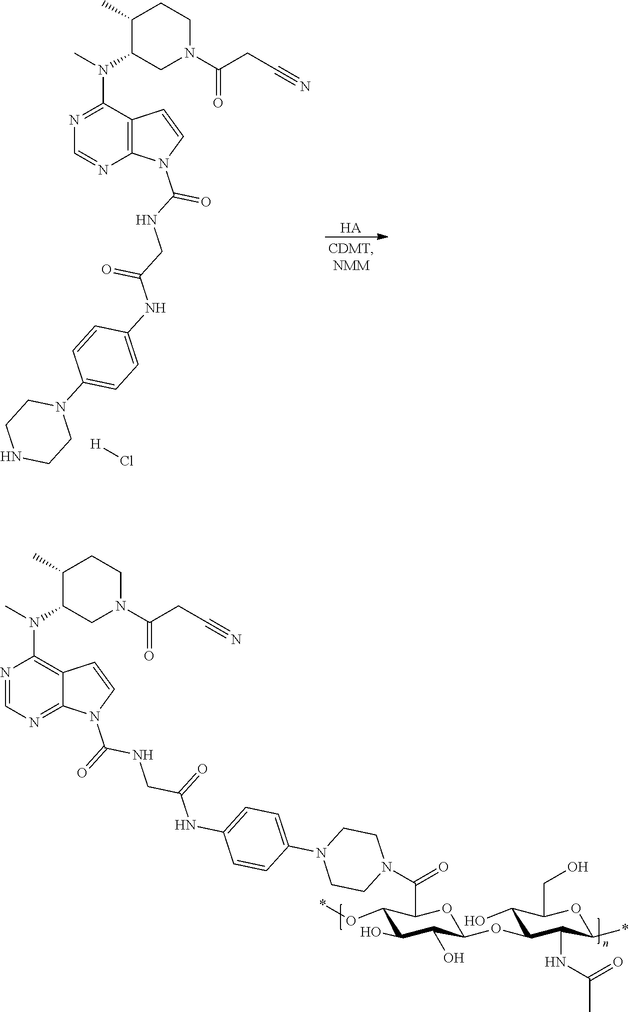

Following Step 3 of Example 1, sodium hyaluronate (MW 50 KDa, 99.54 mg, 0.247 mmol) and 4-[[(3R,4R)-1-(2-cyanoacetyl)-4-methyl-3-piperidyl]-methyl-amino]-N-[2-oxo-2-(4-piperazin-1-ylanilino)ethyl]pyrrolo[2,3-d]pyrimidine-7-carboxamide hydrochloride (150 mg, 0.247 mmol) afforded the title compound 0.121 g, Yield: 49%, DSR: 25%; ¹H NMR (400 MHz, D₂O) δ ppm 8.27-8.09 (m, 0.25H), 7.62-6.96 (m, 0.25H), 6.79-6.64 (m, 0.25H), 4.57-4.24 (m, 2.15H), 4.09-2.77 (m, 14.25H), 2.41-2.31 (m, 0.25H), 2.16-1.45 (m, 4.25H), 1.24-1.12 (m, 0.25H), 1.07-0.78 (m, 0.75H).

With Step 3 of Example 1, reaction of sodium hyaluronate (MW 2000 KDa) provided corresponding product (0.107 g, Yield: 43.6%, DSR: 35%).

### Example 48

### Preparation of conjugate of 4-[[(3R,4R)-1-(2-cyanoacetyl)-4-methyl-3-piperidyl]-methyl-amino]-N-[2-oxo-2-[(5-piperazin-1-yl-2-pyridyl)amino]ethyl]pyrrolo[2,3-d]pyrimidine-7-carboxamide and HA

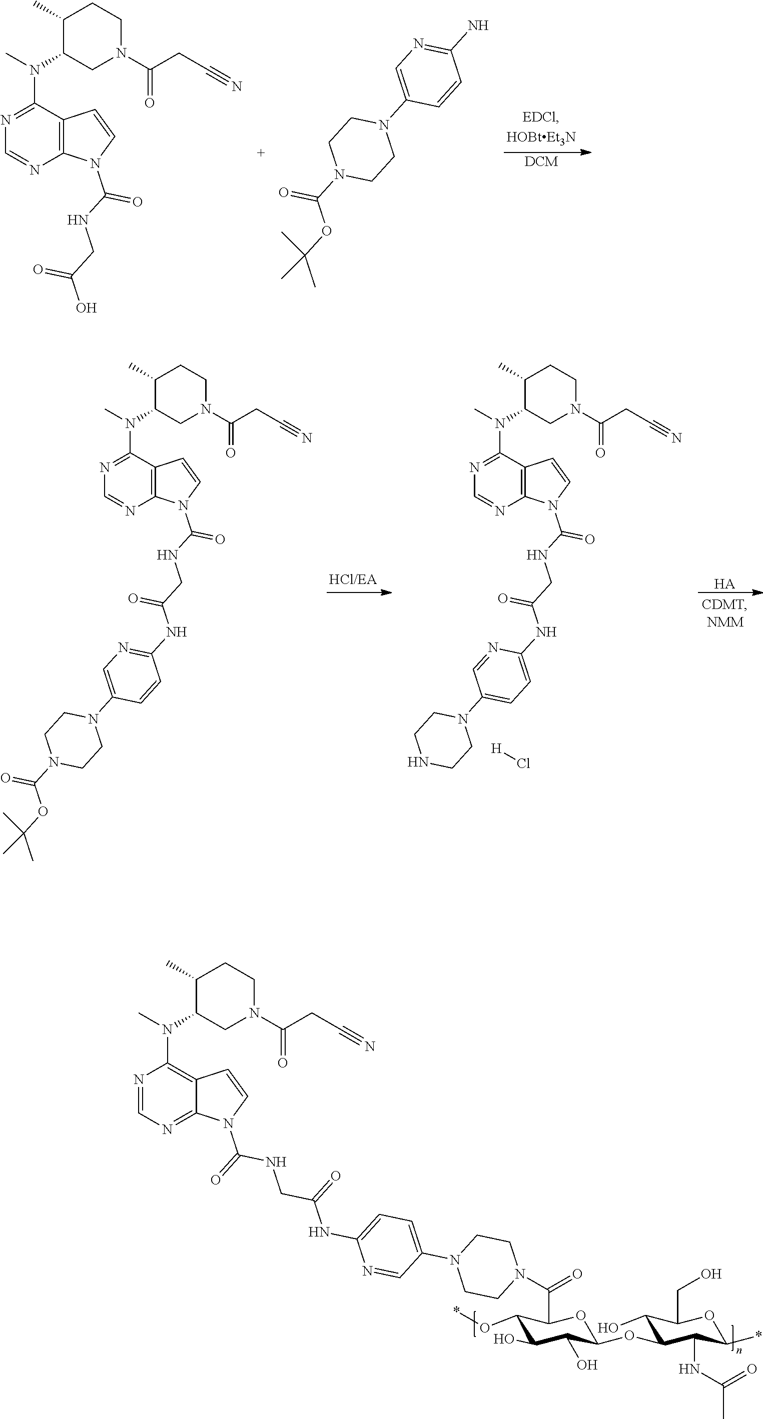

### Step 1: Preparation of tert-butyl 4-[6-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]-3-pyridyl]piperazine-1-carboxylate

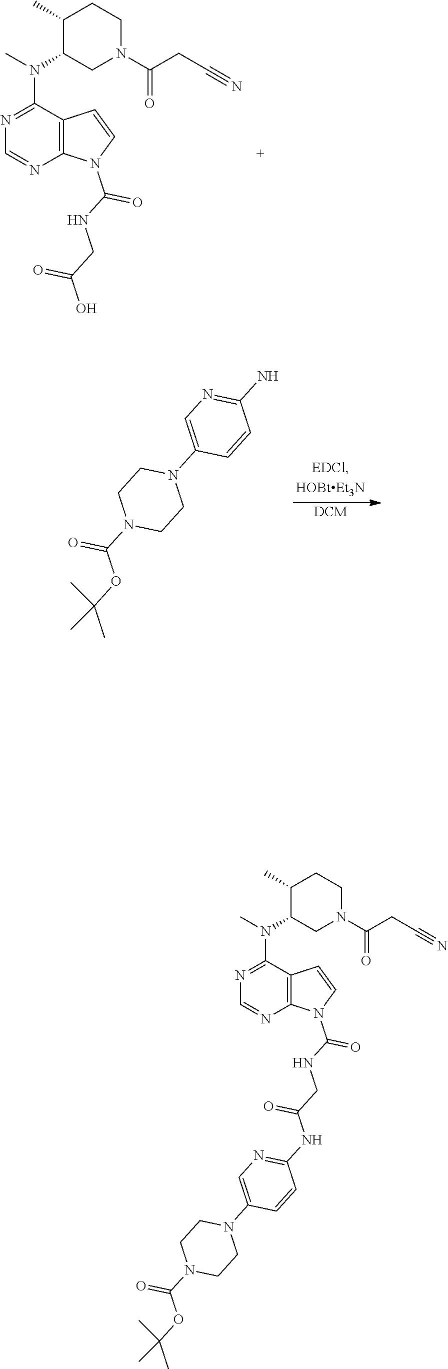

2-[[4-[[(3R,4R)-1-(2-cyanoacetyl)-4-methyl-3-piperidyl]-methyl-amino]pyrrolo[2,3-d]pyrimidine-7-carbonyl]amino]acetic acid (619.5 mg, 1.5 mmol), tert-Butyl 4-(6-aminopyridin-3-yl)piperazine-1-carboxylate (626 mg, 2.25 mmol), 1-ethyl-3-(3-dimethylaminopropyl)carbodiimide (EDCI, 431.3 mg, 2.25 mmol) and 1-hydroxybenzotriazole (HOBt, 304 mg, 2.25 mmol) were dissolved in dichloromethane (20 mL) and triethylamine (0.624 mL, 4.5 mmol) was added. The reaction mixture was stirred at room temperature for 16 h. The solution was diluted with dichloromethane and washed with water and saturated brine solution. The organic layer was dried over sodium sulfate, filtered and then concentrated under reduced pressure. The crude residue was purified by column chromatography (Methanol/dichloromethane=1:100-1:50) to afford the title compound (0.4 g, Yield: 39.6%). MS (m/z): [M+H]+ calcd for C₃₃H₄₃N₁₁O₅, 674.34; found, 674.3. ¹H NMR (400 MHz, CDCl₃) δ ppm 10.54-10.40 (m, 1H), 8.55 (d, J=20.7 Hz, 1H), 8.34 (d, J=14.9 Hz, 1H), 8.12 (t, J=8.1 Hz, 1H), 7.93 (d, J=2.8 Hz, 1H), 7.70 (d, J=4.1 Hz, 1H), 7.33-7.28 (m, 1H), 6.62 (d, J=3.7 Hz, 1H), 5.15 (s, 1H), 4.33 (d, J=17.0 Hz, 2H), 4.14-3.77 (m, 2H), 3.73-3.45 (m, 8H), 3.39 (d, J=18.0 Hz, 3H), 3.18-2.99 (m, 4H), 2.52 (dd, J=16.9, 10.4 Hz, 1H), 2.01-1.85 (m, 1H), 1.83-1.69 (m, 1H), 1.48 (s, 9H), 1.10 (dd, J=12.7, 7.1 Hz, 3H).

### Step 2: Preparation of 4-[[(3R,4R)-1-(2-cyanoacetyl)-4-methyl-3-piperidyl]-methyl-amino]-N-[2-oxo-2-[(5-piperazin-1-yl-2-pyridyl)amino]ethyl]pyrrolo[2,3-d]pyrimidine-7-carboxamide hydrochloride

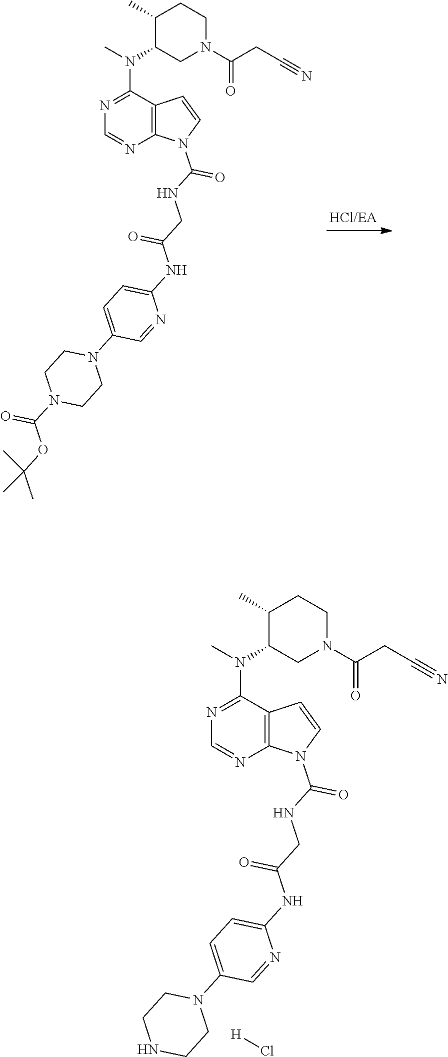

Following Step 2 of Example 1, tert-butyl 4-[6-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]-3-pyridyl]piperazine-1-carboxylate (0.36 g, 0.535 mmol) afforded the title compound as a white solid (0.32 g, Yield: 98%). MS (m/z): [M+H]+ calcd for C₂₈H₃₅N₁₁O₃, 574.29; found, 574.3.

### Step 3: Preparation of conjugate of 4-[[(3R,4R)-1-(2-cyanoacetyl)-4-methyl-3-piperidyl]-methyl-amino]-N-[2-oxo-2-[(5-piperazin-1-yl-2-pyridyl)amino]ethyl]pyrrolo[2,3-d]pyrimidine-7-carboxamide and HA

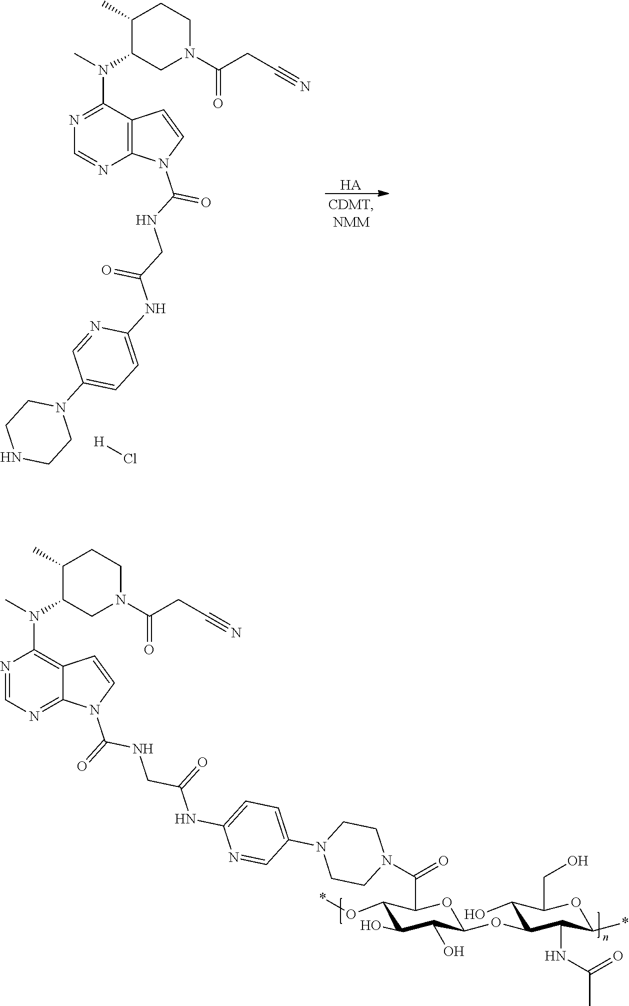

Following Step 3 of Example 1, sodium hyaluronate (MW 50 KDa, 99.14 mg, 0.246 mmol) and 4-[[(3R,4R)-1-(2-cyanoacetyl)-4-methyl-3-piperidyl]-methyl-amino]-N-[2-oxo-2-[(5-piperazin-1-yl-2-pyridyl)amino]ethyl]pyrrolo[2,3-d]pyrimidine-7-carboxamide hydrochloride (150 mg, 0.246 mmol) afforded the title compound 0.126 g, Yield: 52.5%, DSR: 22%; ¹H NMR (400 MHz, D₂O) δ ppm 8.26-8.07 (m, 0.22H), 8.01-7.82 (m, 0.22H), 7.71-7.40 (m, 0.44H), 6.81-6.57 (m, 0.22H), 4.64-4.28 (m, 2.22H), 4.20-2.73 (m, 13.74H), 2.42-2.32 (m, 0.22H), 2.20-1.54 (m, 3.44H), 1.09-0.78 (m, 0.66H).

With Step 3 of Example 1, reaction of sodium hyaluronate (MW 2000 KDa) provided corresponding product (0.116 g, Yield: 48.3%, DSR: 26%).

### Example 49

### Preparation of conjugate of 2-[[4-[[(3R,4R)-1-(2-cyanoacetyl)-4-methyl-3-piperidyl]-methyl-amino]pyrrolo[2,3-d]pyrimidine-7-carbonyl]amino]acetic acid and HA

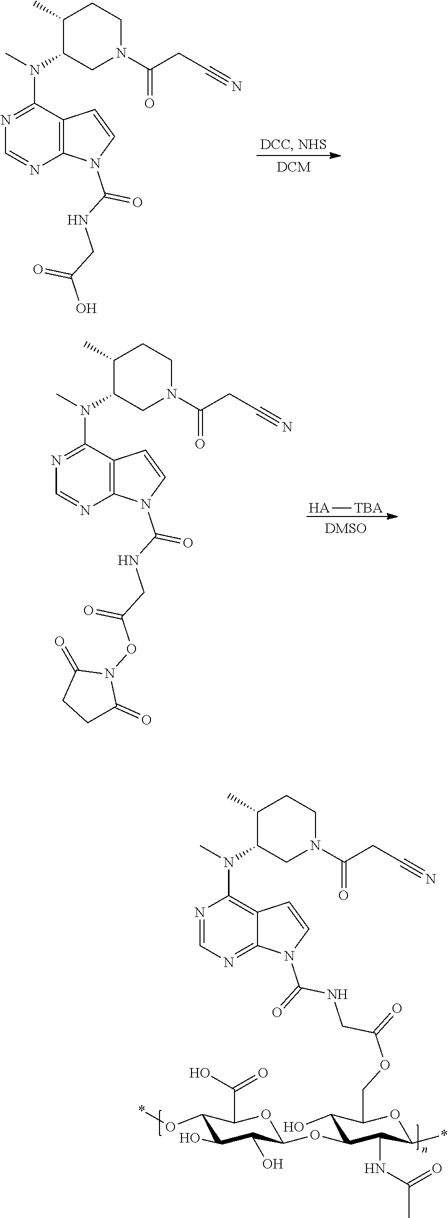

### Step 1: Preparation of (2,5-dioxopyrrolidin-1-yl) 2-[[4-[[(3R,4R)-1-(2-cyanoacetyl)-4-methyl-3-piperidyl]-methyl-amino]pyrrolo[2,3-d]pyrimidine-7-carbonyl]amino]acetate

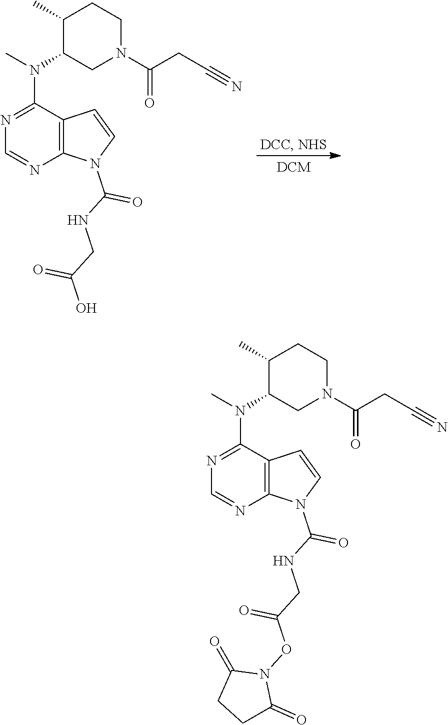

To a stirred mixture of 2-[[4-[[(3R,4R)-1-(2-cyanoacetyl)-4-methyl-3-piperidyl]-methyl-amino]pyrrolo[2,3-d]pyrimidine-7-carbonyl]amino]acetic acid (826 mg, 2 mmol) and 1-Hydroxypyrrolidine-2,5-dione (276 mg, 2.4 mmol) in dichloromethane (20 mL) was slowly added a solution of N,N-dicyclohexylcarbodiimide (494.4 mg, 2.4 mmol) in dichloromethane (20 mL) at ice-bath. The reaction mixture was stirred at room temperature for 16 h. The reaction mixture was filtered and then concentrated under reduced pressure to afford the title compound (1 g, Yield: 98%) that was used without further purification. MS (m/z): [M+H]+ calcd for C₂₃H₂₆N₈O₆, 511.19; found, 511.1.

### Step 2: Preparation of conjugate of 2-[[4-[[(3R,4R)-1-(2-cyanoacetyl)-4-methyl-3-piperidyl]-methyl-amino]pyrrolo[2,3-d]pyrimidine-7-carbonyl]amino]acetic acid and HA

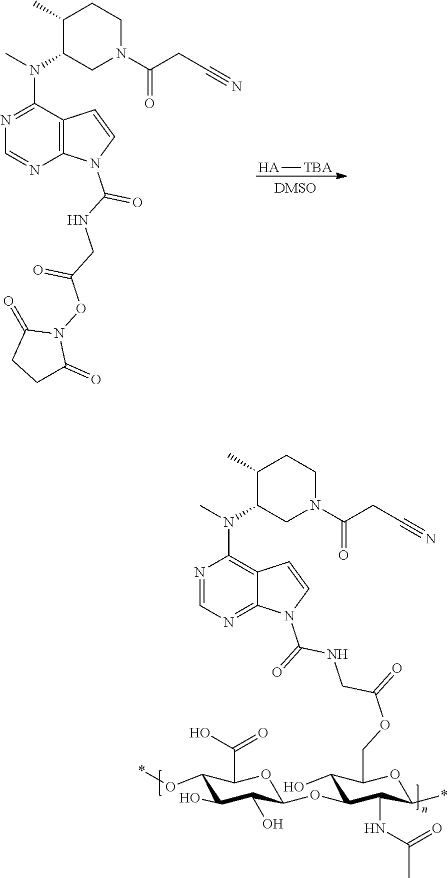

Convert Sodium Hyaluronic acid to TBA hyaluronic acid. The strongly acidic ion exchange resin Amberlite 732 was added to an aqueous solution of the sodium hyaluronate, and the mixture was stirred at room temperature for 8 h. The solution was filtered and subsequently the filtrate was neutralized with an aqueous solution of tetrabutylammonium hydroxide (TBA-OH). The resulting aqueous solution was instantly frozen and lyophilized to TBA salt of hyaluronic acid (HA-TBA).

To a stirred mixture of TBA salt of hyaluronic acid (HA-TBA, 331 mg, 0.5 mmol in anhydrous DMSO (20 mL) was slowly added triethylamine (0.12 mL, 0.875 mmol) at 0-15° C. (2,5-dioxopyrrolidin-1-yl) 2-[[4-[[(3R,4R)-1-(2-cyanoacetyl)-4-methyl-3-piperidyl]-methyl-amino]pyrrolo[2,3-d]pyrimidine-7-carbonyl]amino]acetate (255 mg, 0.5 mmol) in anhydrous DMSO (20 mL) was then added to the reaction mixture at 0-15° C. The reaction mixture was stirred for 16 h at room temperature. A 2.5 wt % sodium chloride solution (7 mL) was then added to the reaction mixture, which was stirred for 1 h and followed by the dropwise addition of acetone (250 mL) while stirring. The mixture was filtered. The filter cake was dissolved in 80 mL of deionized water and acetonitrile (V/V=3:1). Extensive dialysis (3.5 kDa MW cutoff) of the solution against deionized water and lyophilization afforded the title compound. sodium hyaluronate MW 10 kDa MW 0.17 g, Yield: 43.9%, DSR: 20%; ¹H NMR (400 MHz, D₂O) δ ppm 8.26-8.12 (m, 0.2H), 7.62-7.46 (m, 0.2H), 6.80-6.67 (m, 0.2H), 4.54-4.18 (m, 2.2H), 4.09-2.94 (m, 12.2H), 2.41-2.38 (m, 0.2H), 2.12-1.58 (m, 3.4H), 1.09-0.87 (m, 0.6H).

With this step, reaction of TBA hyaluronate (MW 50 KDa) provided corresponding product (0.245 g, Yield: 613.3%, DSR: 6%).

With this step, reaction of TBA hyaluronate (MW 500 KDa) provided corresponding product (0.274 g, Yield: 70.8%, DSR: 23%)

With this step, reaction of TBA hyaluronate (MW 2000 KDa) provided corresponding product (0.237 g, Yield: 61.2%, DSR: 24%).

### Example 50

### Preparation of conjugate of 4-[[4-[[(3R,4R)-1-(2-cyanoacetyl)-4-methyl-3-piperidyl]-methyl-amino]pyrrolo[2,3-d]pyrimidine-7-carbonyl]amino]butanoic acid and HA

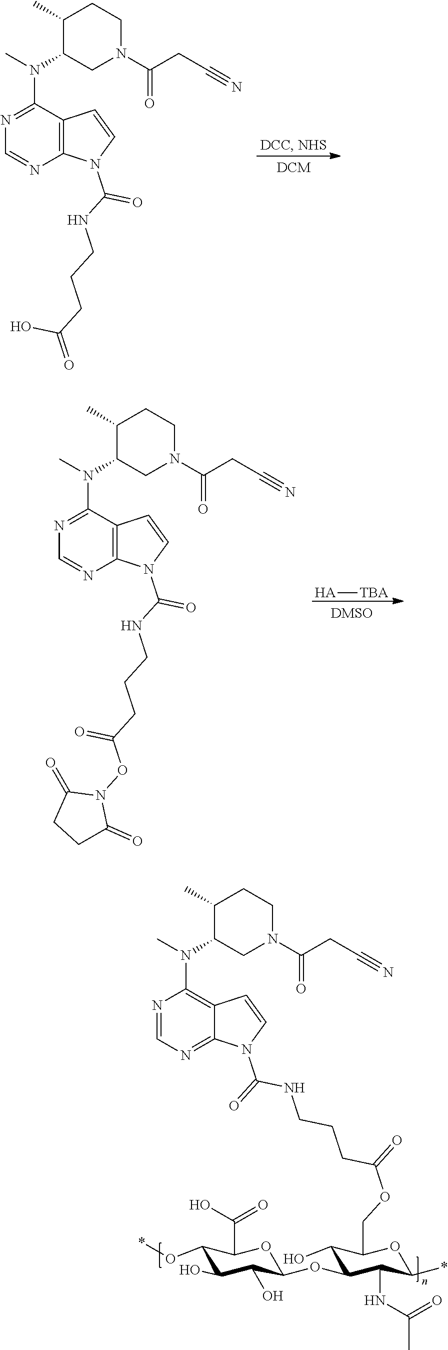

### Step 1: Preparation of (2,5-dioxopyrrolidin-1-yl) 4-[[4-[[(3R,4R)-1-(2-cyanoacetyl)-4-methyl-3-piperidyl]-methyl-amino]pyrrolo[2,3-d]pyrimidine-7-carbonyl]amino]butanoate

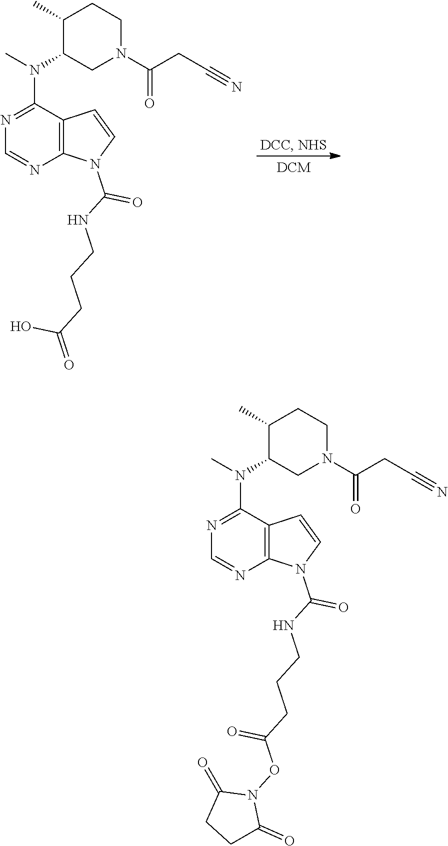

To a stirred mixture of 4-[[4-[[(3R,4R)-1-(2-cyanoacetyl)-4-methyl-3-piperidyl]-methyl-amino]pyrrolo[2,3-d]pyrimidine-7-carbonyl]amino]butanoic acid (882 mg, 2 mmol) and 1-Hydroxypyrrolidine-2,5-dione (276 mg, 2.4 mmol) in dichloromethane (25 mL) was slowly added a solution of N,N-Dicyclohexylcarbodiimide (494.4 mg, 2.4 mmol) in dichloromethane (15 mL) at ice-bath. The reaction mixture was stirred at room temperature for 16 h. The reaction mixture was filtered and then concentrated under reduced pressure to afford the title compound (1 g, Yield: 93%) that was used without further purification. MS (m/z): [M+H]+ calcd for C₂₅H₃₀N₈O₆, 538.22; found, 538.1.

### Step 2: Preparation of conjugate of 4-[[4-[[(3R,4R)-1-(2-cyanoacetyl)-4-methyl-3-piperidyl]-methyl-amino]pyrrolo[2,3-d]pyrimidine-7-carbonyl]amino]butanoic acid and HA

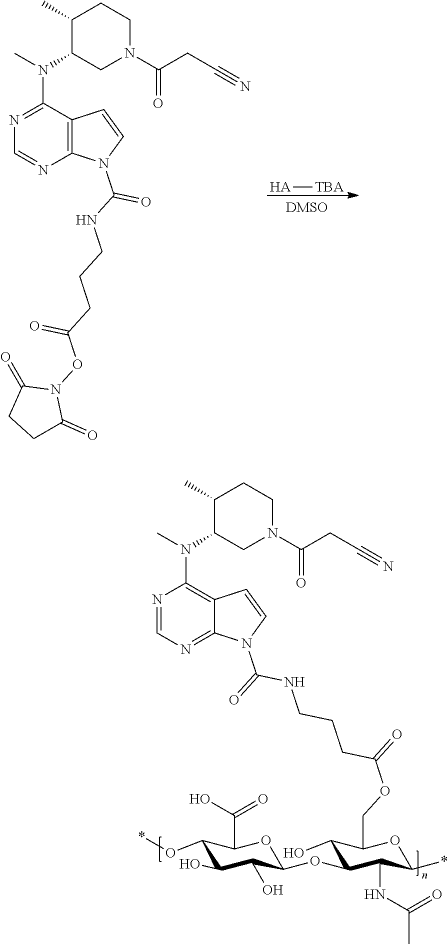

To a stirred mixture of TBA salt of hyaluronic acid (HA-TBA, 331 mg, 0.5 mmol in anhydrous DMSO (20 mL) was slowly added triethylamine (0.12 mL, 0.875 mmol) at 0-15° C. (2,5-dioxopyrrolidin-1-yl) 4-[[4-[[(3R,4R)-1-(2-cyanoacetyl)-4-methyl-3-piperidyl]-methyl-amino]pyrrolo[2,3-d]pyrimidine-7-carbonyl]amino]butanoate (269 mg, 0.5 mmol) in anhydrous DMSO (20 mL) was then added to the reaction mixture at 0-15° C. The reaction mixture was stirred for 16 h at room temperature. 7 mL of a 2.5 wt % sodium chloride solution was then added to the reaction mixture, which was stirred for 1 h and followed by the dropwise addition of acetone (250 mL) while stirring. The mixture was filtered. The filter cake was dissolved in 80 mL of deionized water and acetonitrile (V/V=3:1). Extensive dialysis (3.5 kDa MW cutoff) of the solution against deionized water and lyophilization afforded the title compound. sodium hyaluronate MW 10 kDa, 0.166 g, Yield: 41.3%, DSR: 10%; ¹H NMR (400 MHz, D₂O) δ ppm 8.26-8.09 (m, 0.1H), 7.61-7.44 (m, 0.1H), 6.78-6.65 (m, 0.1H), 4.60-4.22 (m, 2.1H), 4.06-2.94 (m, 11.1H), 2.55-2.31 (m, 0.3H), 2.15-1.57 (m, 3.4H), 1.11-0.86 (m, 0.3H). sodium hyaluronate MW 50 kDa, 0.181 g, Yield: 45.1%, DSR: 14%;

With this step, reaction of TBA hyaluronate (MW 500 KDa) provided corresponding product (0.263 g, Yield: 65.5%, DSR: 5%),

With this step, reaction of TBA hyaluronate (MW 2000 KDa) provided corresponding product (0.17 g, Yield: 42.3%, DSR: 7%).

### Example 51

### Preparation of conjugate of 4-[[4-[[(3R,4R)-1-(2-cyanoacetyl)-4-methyl-3-piperidyl]-methyl-amino]pyrrolo[2,3-d]pyrimidine-7-carbonyl]amino]benzoic acid and HA

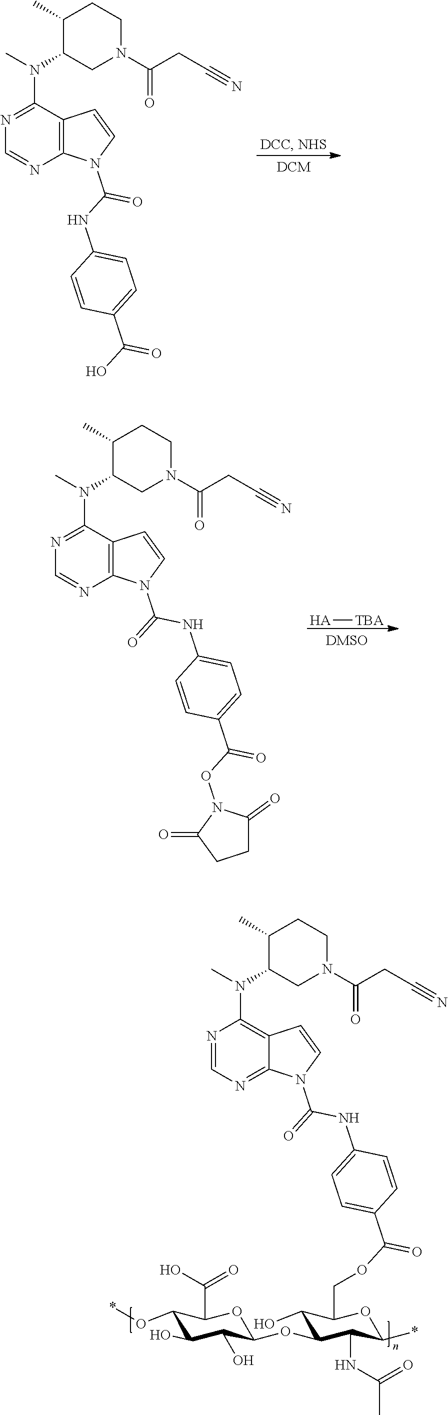

### Step 1: Preparation of (2,5-dioxopyrrolidin-1-yl) 4-[[4-[[(3R,4R)-1-(2-cyanoacetyl)-4-methyl-3-piperidyl]-methyl-amino]pyrrolo[2,3-d]pyrimidine-7-carbonyl]amino]benzoate

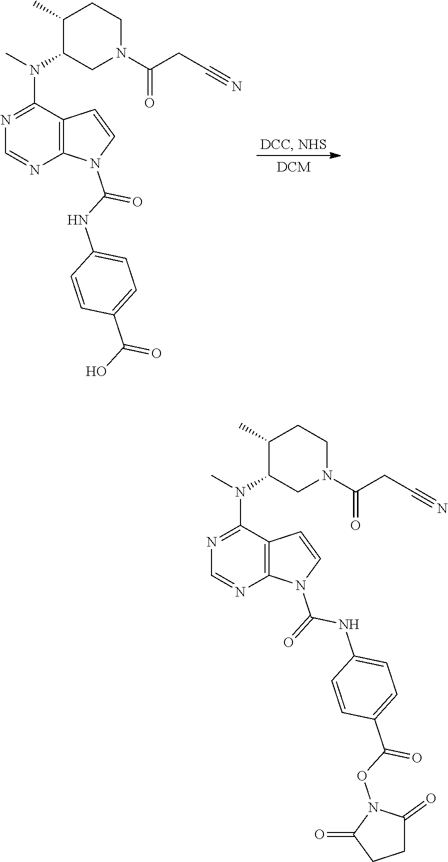

To a stirred mixture of 4-[[4-[[(3R,4R)-1-(2-cyanoacetyl)-4-methyl-3-piperidyl]-methyl-amino]pyrrolo[2,3-d]pyrimidine-7-carbonyl]amino]benzoic acid (950 mg, 2 mmol) and 1-hydroxypyrrolidine-2,5-dione (276 mg, 2.4 mmol) in dichloromethane (40 mL) was slowly added a solution of N,N-dicyclohexylcarbodiimide (494.4 mg, 2.4 mmol) in dichloromethane (20 mL) at ice-bath. The reaction mixture was stirred at room temperature for 16 h. The reaction mixture was filtered and then concentrated under reduced pressure to afford the title compound (1.1 g, Yield: 96%) that was used without further purification. MS (m/z): [M+H]+ calcd for C₂₈H₂₈N₈O₆, 573.21; found, 573.1.

### Step 2: Preparation of conjugate of 4-[[4-[[(3R,4R)-1-(2-cyanoacetyl)-4-methyl-3-piperidyl]-methyl-amino]pyrrolo[2,3-d]pyrimidine-7-carbonyl]amino]benzoic acid and HA

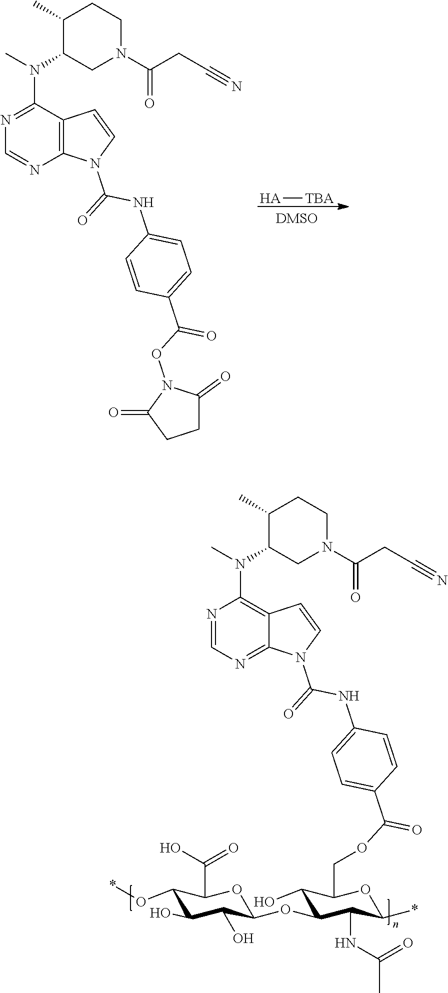

To a stirred mixture of TBA salt of hyaluronic acid (HA-TBA, 331 mg, 0.5 mmol in anhydrous DMSO (20 mL) was slowly added triethylamine (0.12 mL, 0.875 mmol) at 0-15° C. (2,5-dioxopyrrolidin-1-yl) 4-[[4-[[(3R,4R)-1-(2-cyanoacetyl)-4-methyl-3-piperidyl]-methyl-amino]pyrrolo[2,3-d]pyrimidine-7-carbonyl]amino]benzoate (286 mg, 0.5 mmol) in anhydrous DMSO (20 mL) was then added to the reaction mixture at 0-15° C. The reaction mixture was stirred for 16 h at room temperature. 7 mL of a 2.5 wt % sodium chloride solution was then added to the reaction mixture, which was stirred for 1 h and followed by the dropwise addition of acetone (250 mL) while stirring. The mixture was filtered. The filter cake was dissolved in 80 mL of deionized water and acetonitrile (V/V=3:1). Extensive dialysis (3.5 kDa MW cutoff) of the solution against deionized water and lyophilization afforded the title compound. sodium hyaluronate MW 10 kDa, 0.177: 42.3%, DSR: 10%; ¹H NMR (400 MHz, D₂O) δ ppm 8.09-7.92 (m, 0.2H), 7.86-7.69 (m, 0.2H), 7.54-7.32 (m, 0.3H), 6.83-6.74 (m, 0.1H), 4.58-4.22 (m, 2.1H), 4.05-3.05 (m, 10.9H), 2.39-2.27 (m, 0.1H), 2.12-1.65 (m, 3.2H), 1.11-0.92 (m, 0.3H). sodium hyaluronate MW 50 kDa, 0.2 g, Yield: 47.8%, DSR: 6%;

With this step, reaction of TBA hyaluronate (MV 500 KDa) provided corresponding product (0.187 g, Yield: 44.7%, DSR: 5%),

With This step, reaction of TBA hyaluronate (MW 2000 KDa) provided corresponding product (0.1 g, Yield: 23.9%, DSR: 2%).

### Example 52

### Preparation of conjugate of O-(4-aminophenyl) 4-(((3R,4R)-1-(2-cyanoacetyl)-4-methylpiperidin-3-yl)(methyl)amino)-7H-pyrrolo[2,3-d]pyrimidine-7-carbothioate and HA

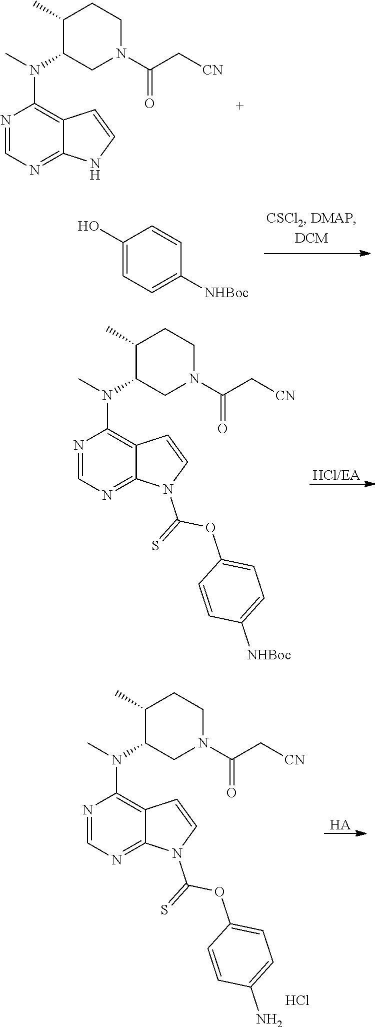

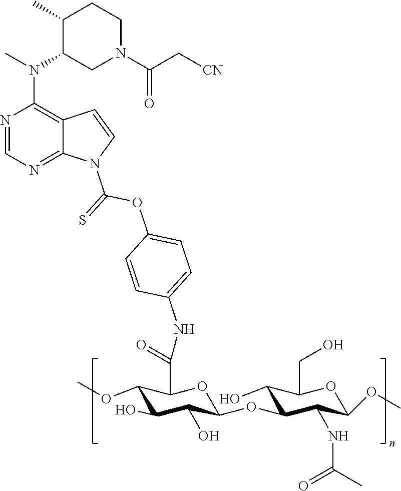

### Step 1: Preparation of O-(4-((tert-butoxycarbonyl)amino)phenyl) 4-(((3R,4R)-1-(2-cyanoacetyl)-4-methylpiperidin-3-yl)(methyl)amino)-7H-pyrrolo[2,3-d]pyrimidine-7-carbothioate

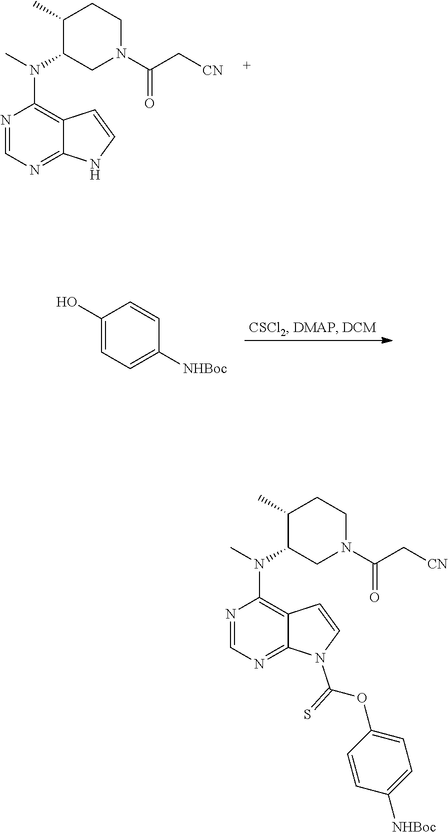 [SMILES: C[C@@H]1CCN(C(=O)CC#N)C[C@@H]1N(C)c1ncnc2[nH]ccc12 ; CC(C)(C)OC(=O)Nc1ccc(O)cc1 ; C[C@@H]1CCN(C(=O)CC#N)C[C@@H]1N(C)c1ncnc2c1ccn2C(=S)Oc1ccc(NC(=O)OC(C)(C)C)cc1]

To a mixture of tert-butyl (4-hydroxyphenyl)carbamate (2 g, 9.56 mmol, 1 eq) and N,N-dimethylpyridin-4-amine (2.92 g, 23.9 mmol, 2.5 eq) in dichloromethane (40 mL, 20V) was added thiophosgene (1.21 g, 10.5 mmol, 1.1 eq) under N₂, the reaction mixture was stirred at room temperature for 0.5 h. Then tofacitinib (2.99 g, 9.56 mmol, 1 eq) was added-into, the resulting mixture was stirred at room temperature for another 12 h. After most of tofacitinib was consumed, the solvent was removed under reduced pressure, the residue was purified by silical gel chromatography to give the title product (1.827 g, yield: 34%); MS (m/z): [M+H]+ calcd for C₂₈H₃₃N₇O₄S, 564.23; found, 564.1.

### Step 2: Preparation of O-(4-aminophenyl) 4-(((3R,4R)-1-(2-cyanoacetyl)-4-methyl piperidin-3-yl)(methyl)amino)-7H-pyrrolo[2,3-d]pyrimidine-7-carbothioate hydrochloride

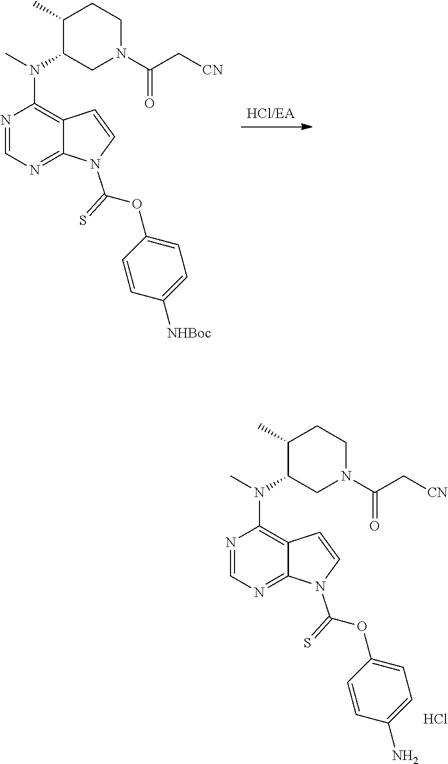 [SMILES: C[C@@H]1CCN(C(=O)CC#N)C[C@@H]1N(C)c1ncnc2c1ccn2C(=S)Oc1ccc(NC(=O)OC(C)(C)C)cc1 ; C[C@@H]1CCN(C(=O)CC#N)C[C@@H]1N(C)c1ncnc2c1ccn2C(=S)Oc1ccc(N)cc1 ; Cl]

Following Step 2 of Example 1, 0-(4-((tert-butoxycarbonyl)amino)phenyl) 4-(((3R,4R)-1-(2-cyanoacetyl)-4-methylpiperidin-3-yl)(methyl)amino)-7H-pyrrolo[2,3-d]pyrimidine-7-carbothioate (1.5 g, 2.661 mmol, 1 eq) gave the desired product as HCl salt (1.33 g, yield: 100%); MS (m/z): [M+H]+ calcd for C₂₃H₂₅N₇O₂S, 64.18; found, 464.1. ¹H-NMR (400 MHz, D₂O) δ ppm 8.45-8.30 (m, 1H), 7.66-7.44 (m, 2H), 7.44-7.22 (m, 2H), 7.09-6.74 (m, 2H), 4.73 (s, 1H), 4.06-3.94 (m, 3H), 3.71-3.17 (m, 6H), 2.56 (s, 1H), 2.03-1.70 (m, 2H), 1.13 (dd, J=14.8, 6.8 Hz, 3H).

### Step 3: Preparation of conjugate of O-(4-aminophenyl)4-(((3R,4R)-1-(2-cyanoacetyl)-4-methylpiperidin-3-yl)(methyl)amino)-7H-pyrrolo[2,3-d]pyrimidine-7-carbothioate and HA

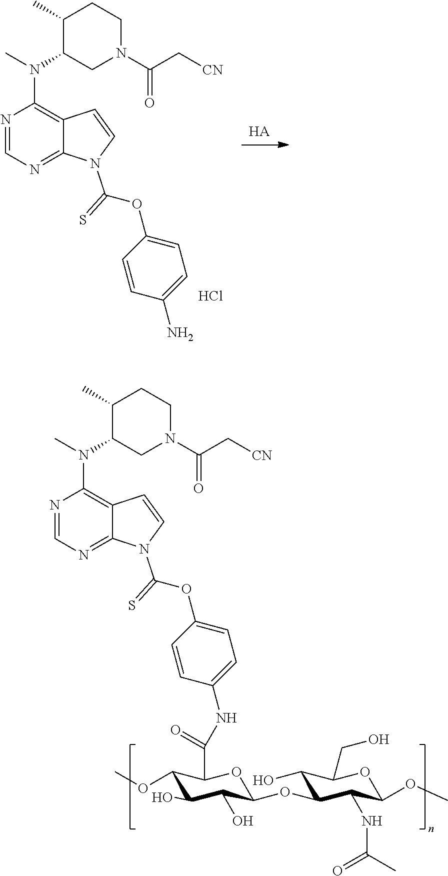

Following Step 3 of Example 1, sodium hyaluronate (MW 50 KDa, 0.161 g, 0.4 mmol, 1 eq) and O-(4-aminophenyl) 4-(((3R,4R)-1-(2-cyanoacetyl)-4-methylpiperidin-3-yl)(methyl)amino)-7H-pyrrolo[2,3-d]pyrimidine-7-carbothioate hydrochloride (0.2 g, 0.4 mmol, 1 eq) afforded the title compound (0.15 g, yield: 45%, DSR=14.3%); ¹H-NMR (400 MHz, D₂O) δ ppm 7.7-7.55 (m, 0.5H), 7.35-7.2 (m, 0.5H), 4.6-4.45 (m, 1.43H), 3.85-3.4 (m, 12H), 2.46-2.43 (m, 0.14H), 2.00 (s, 3.0H), 1.9-1.45 (m, 2.86H), 1.3-1.25 (m, 0.43H).

With This procedure, reaction of sodium hyaluronate (MW 2000 KDa) provided corresponding product (0.175 g, yield: 52%, DSR=4.6%).

### Example 53

### Preparation of conjugate of O-(piperidin-4-yl) 4-(((3R,4R)-1-(2-cyano acetyl)-4-methylpiperidin-3-yl)(methyl)amino)-7H-pyrrolo[2,3-d]pyrimidine-7-carbothioate and HA

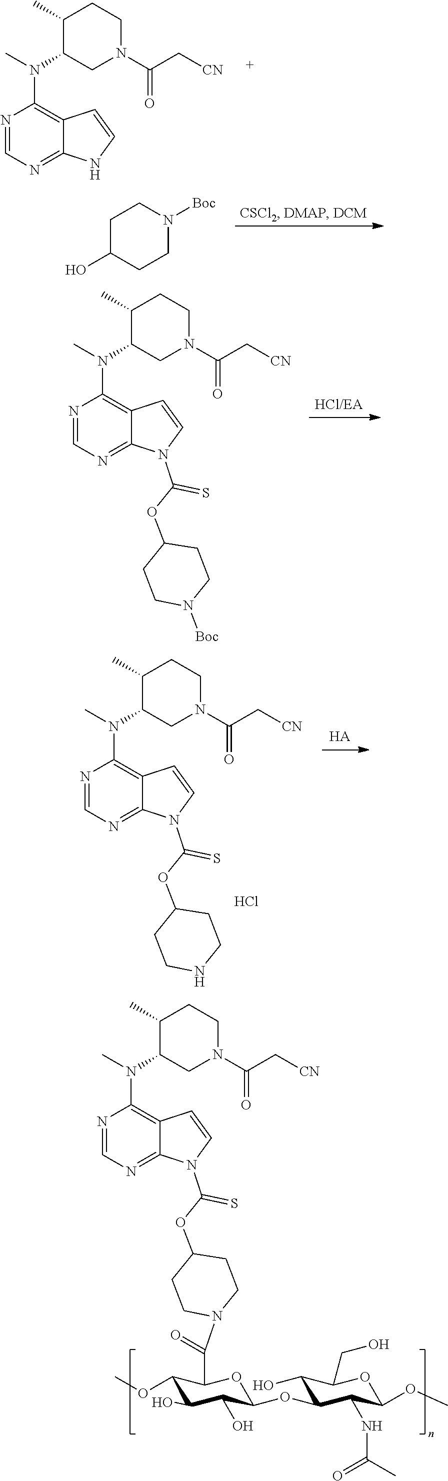

### Step 1: Preparation of tert-butyl 4-((4-(((3R,4R)-1-(2-cyanoacetyl)-4-methylpiperidin-3-yl)(methyl)amino)-7H-pyrrolo[2,3-d]pyrimidine-7-carbonothioyl)oxy)piperidine-1-carboxylate

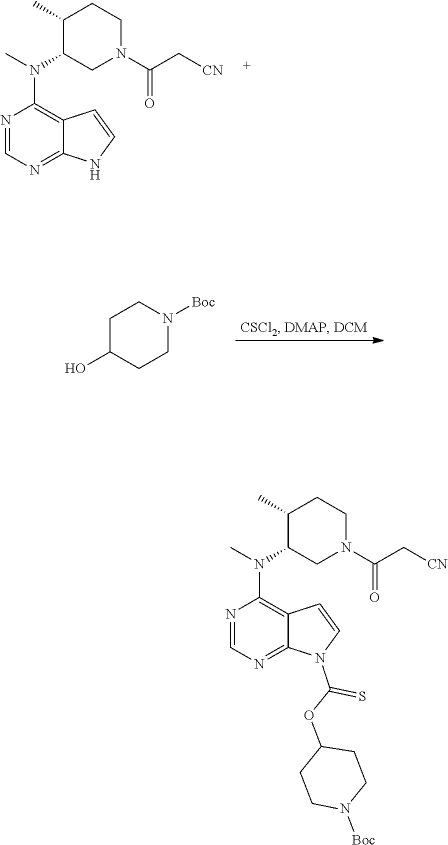 [SMILES: C[C@@H]1CCN(C(=O)CC#N)C[C@@H]1N(C)c1ncnc2[nH]ccc12 ; CC(C)(C)OC(=O)N1CCC(O)CC1 ; C[C@@H]1CCN(C(=O)CC#N)C[C@@H]1N(C)c1ncnc2c1ccn2C(=S)OC1CCN(C(=O)OC(C)(C)C)CC1]

To a mixture of tert-butyl 4-hydroxypiperidine-1-carboxylate (2 g, 9.94 mmol, 1 eq) and N,N-dimethyl pyridin-4-amine (3.03 g, 9.94 mmol, 2.5 eq) in dichloromethane (40 mL, 20V) was added thiophosgene (1.26 g, 10.934 mmol, 1.1 eq) under N₂, the reaction mixture was stirred at room temperature for 0.5 h. Then tofacitinib (3.1 g, 9.94 mmol, 1 eq) was added to, the resulting mixture was stirred at room temperature for another 12 h. After most of tofacitinib was consumed, the solvent was removed under reduced pressure, the residue was purified by silical gel chromatography to give the title product (1.2 g, yield: 22%); MS (m/z): [M+H]+ calcd for C₂₇H₃₇N₇O₄S, 556.26; found, 556.2.

### Step 2: Preparation of O-(piperidin-4-yl) 4-(((3R,4R)-1-(2-cyanoacetyl)-4-methylpiperidin-3-yl) (methyl)amino)-7H-pyrrolo[2,3-d]pyrimidine-7-carbothioate hydrochloride

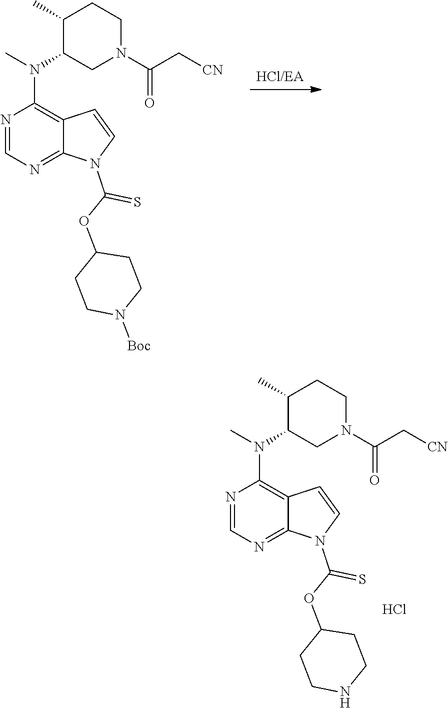 [SMILES: C[C@@H]1CCN(C(=O)CC#N)C[C@@H]1N(C)c1ncnc2c1ccn2C(=S)OC1CCN(C(=O)OC(C)(C)C)CC1 ; C[C@@H]1CCN(C(=O)CC#N)C[C@@H]1N(C)c1ncnc2c1ccn2C(=S)OC1CCNCC1 ; Cl]

Following Step 2 of Example 1, tert-butyl 4-((4-(((3R,4R)-1-(2-cyanoacetyl)-4-methylpiperidin-3-yl)(methyl)amino)-7H-pyrrolo[2,3-d]pyrimidine-7-carbonothioyl)oxy)piperidine-1-carboxylate (1 g, 1.8 mmol, 1 eq) gave the desired product as HCl salt (0.885 g, yield: 100%); MS (m/z): [M+H]+ calcd for C₂₂H₂₉N₇O₂S, 456.21; found, 456.1. ¹H-NMR (400 MHz, D₂O) δ ppm 8.49 (d, J=9.7 Hz, 1H), 8.00 (s, 1H), 7.03 (s, 1H), 5.93 (br, 1H), 5.03 (s, 1H), 4.15-4.0 (m, 1H), 3.79-3.31 (m, 11H), 3.29-3.05 (m, 1H), 2.52 (br, 1H), 2.45-2.28 (m, 4H), 2.0-1.7 (m, 3H), 1.11 (dd, J=17.1, 7.1 Hz, 3H).

### Step 3: Preparation of conjugate of O-(piperidin-4-yl) 4-(((3R,4R)-1-(2-cyanoacetyl)-4-methylpiperidin-3-yl)(methyl)amino)-7H-pyrrolo[2,3-d]pyrimidine-7-carbothioate and HA

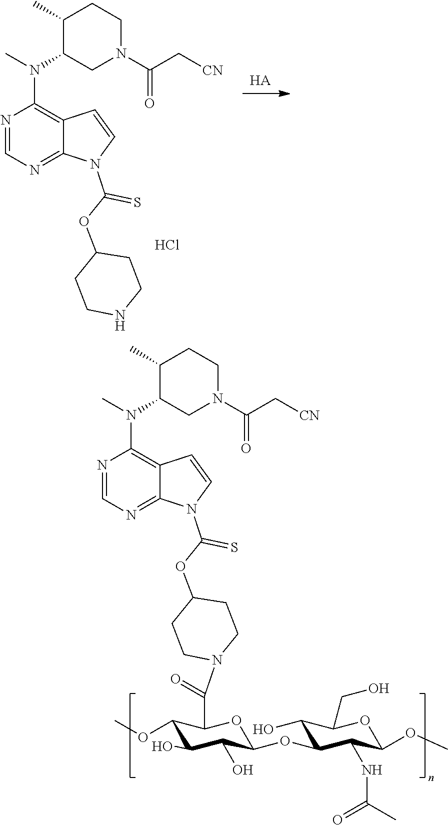

Following Step 3 of Example 1, sodium hyaluronate (MW 50 KDa, 0.164 g, 0.406 mmol, 1 eq) and O-(piperidin-4-yl)4-(((3R,4R)-1-(2-cyanoacetyl)-4-methylpiperidin-3-yl) (methyl)amino)-7H-pyrrolo[2,3-d]pyrimidine-7-carbothioate hydrochloride (0.2 g, 0.406 mmol, 1 eq) afforded the title compound (0.17 g, yield: 34%, DSR=12.6%); ¹H-NMR (400 MHz, D₂O) δ ppm δ 8.25-7.0 (m, 0.22H), 7.0-6.5 (m, 0.08H), 5.95-5.49 (m, 0.08H), 4.45-4.25 (m, 1.26H), 3.8-2.9 (m, 12.63H), 2.33 (br, 0.131H), 1.88 (s, 3H), 1.81-1.72 (m, 0.51H), 1.23-1.07 (m, 0.25H), 0.99-0.82 (m, 0.38H).

With this step, reaction of sodium hyaluronate (MW 2000 KDa) provided corresponding product (0.192 g, yield: 56.6%, DSR=4.6%).

### Example 54

### Preparation of conjugate of 4-aminobutyl ((4-(((3R,4R)-1-(2-cyanoacetyl)-4-methylpiperidin-3-yl)(methyl)amino)-7H-pyrrolo[2,3-d]pyrimidin-7-yl)methyl)carbonate and HA

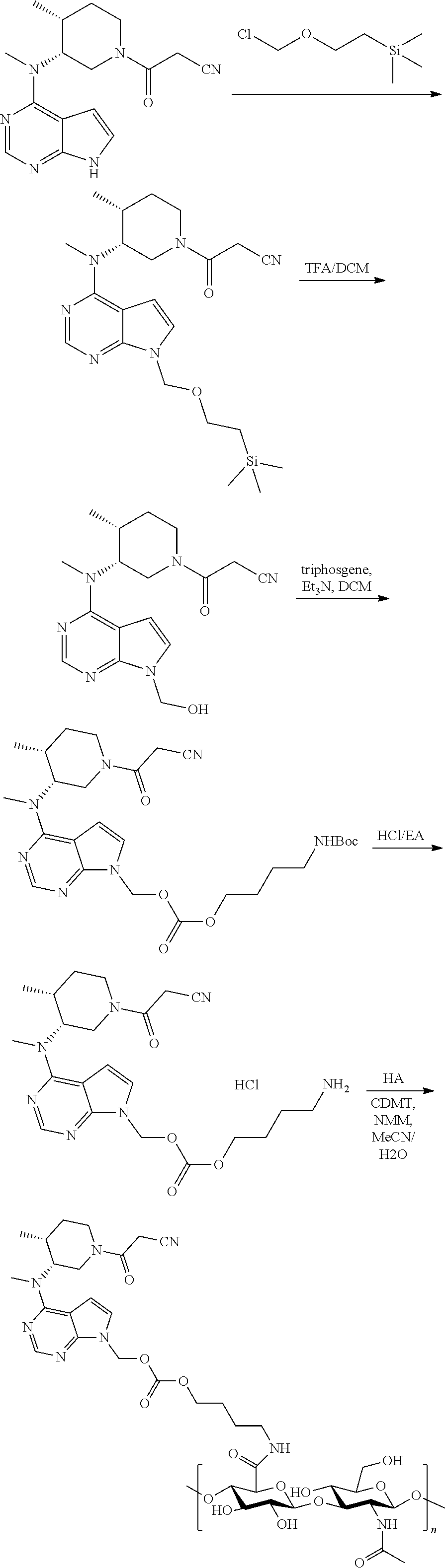

### Step 1: Preparation of 3-((3R,4R)-4-methyl-3-(methyl(7-((2-(trimethylsilyl)ethoxy)methyl)-7H-pyrrolo[2,3-d]pyrimidin-4-yl)amino)piperidin-1-yl)-3-oxopropanenitrile

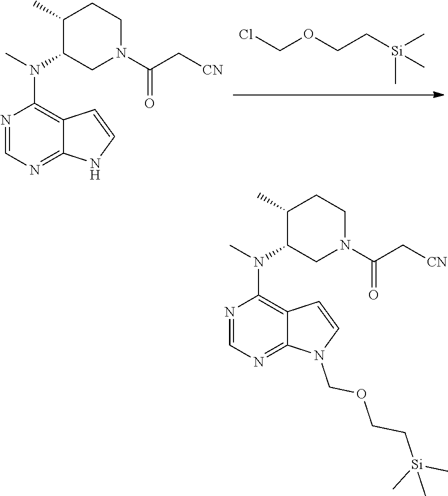

To a mixture of tofacitinib (9 g, 28.81 mmol, 1 eq) in dichloromethane (180 mL, 20V) was added DIPethyl acetate (3.745 g, 28.81 mmol, 1 eq) under N₂, the reaction mixture was stirred at room temperature for 0.5 h. Then (2-(chloromethoxy) ethyl)trimethylsilane (4.8 g, 28.81 mmol, 1 eq) was added to, the resulting mixture was stirred overnight at room temperature. After most of tofacitinib was consumed, the solvent was removed under reduced pressure, the residue was purified by silical gel chromatography to give the title product (9 g, yield: 71%); MS (m/z): [M+H]+ calcd for C₂₂H₃₄N₆O₂Si, 443.25; found, 443.2.

### Step 2: Preparation of 3-((3R,4R)-3-((7-(hydroxymethyl)-7H-pyrrolo[2,3-d]pyrimidin-4-yl)(methyl)amino)-4-methylpiperidin-1-yl)-3-oxopropanenitrile

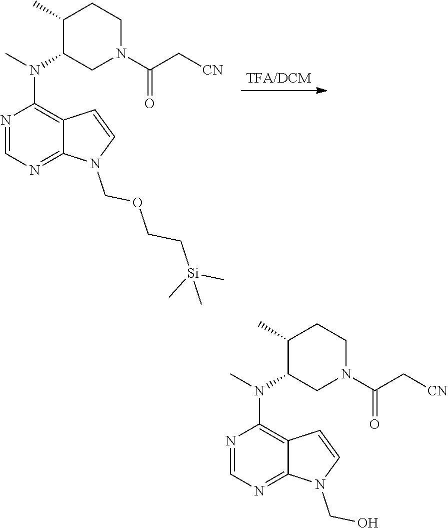 [SMILES: C[C@@H]1CCN(C(=O)CC#N)C[C@@H]1N(C)c1ncnc2c1ccn2COCC[Si](C)(C)C ; C[C@@H]1CCN(C(=O)CC#N)C[C@@H]1N(C)c1ncnc2c1ccn2CO]

To a mixture of 3-((3R,4R)-4-methyl-3-(methyl(7-((2-(trimethylsilyl)ethoxy) methyl)-7H-pyrrolo[2,3-d]pyrimidin-4-yl)amino)piperidin-1-yl)-3-oxopropanenitrile (5 g, 11.3 mmol, 1 eq) in dichloromethane (100 mL, 20V) was added TFA (6.44 g, 56.5 mmol, 5 eq) dropwise under N₂ at 0° C. The resulting reaction mixture was stirred at 0° C. for 30 min, allowed to warm to room temperature then stirred at this temperature for 24 hs. After most of S.M was consumed, NaHCO₃(sat) was added to the above solution to adjust the pH to 8 at 0° C. Then the mixture was poured into separatory funnel and separated. The organic phase was washed with NaCl(sat), dried over Na₂SO₄ and then concentrated to give the title product (3.5 g, yield: 90%); MS (m/z): [M+H]+ calcd for C₁₇H₂₂N₆O₂, 343.18; found, 343.1.

### Step 3: Preparation of tert-butyl (4-((((4-(((3R,4R)-1-(2-cyanoacetyl)-4-methylpiperidin-3-yl) (methyl)amino)-7H-pyrrolo[2,3-d]pyrimidin-7-yl)methoxy) carbonyl)oxy)butyl)carbamate

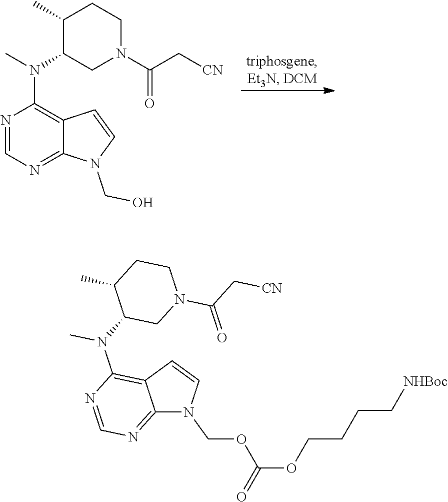 [SMILES: C[C@@H]1CCN(C(=O)CC#N)C[C@@H]1N(C)c1ncnc2c1ccn2CO ; C[C@@H]1CCN(C(=O)CC#N)C[C@@H]1N(C)c1ncnc2c1ccn2COC(=O)OCCCCNC(=O)OC(C)(C)C]

Following Step 1 of Example 1, 3-((3R,4R)-3-((7-(hydroxymethyl)-7H-pyrrolo[2,3-d]pyrimidin-4-yl)(methyl)amino)-4-methylpiperidin-1-yl)-3-oxopropanenitrile (1 g, 2.92 mmol, 1 eq) and tert-butyl (4-hydroxybutyl) carbamate (1 g, 2.92 mmol, 1 eq) gave the title product (0.85 g, yield: 52%); MS (m/z): [M+H]+ calcd for C₂₇H₃₉N₇O₆, 558.30; found, 558.2.

### Step 4: Preparation of 4-aminobutyl ((4-(((3R,4R)-1-(2-cyanoacetyl)-4-methylpiperidin-3-yl)(methyl)amino)-7H-pyrrolo[2,3-d]pyrimidin-7-yl)methyl) carbonate hydrochloride

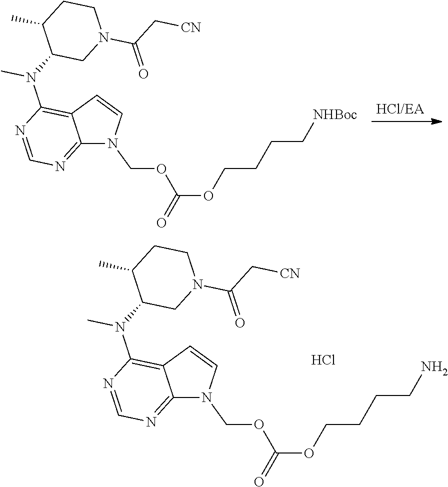

Following Step 2 of Example 1, tert-butyl (4-((((4-(((3R,4R)-1-(2-cyanoacetyl)-4-methylpiperidin-3-yl)(methyl)amino)-7H-pyrrolo[2,3-d]pyrimidin-7-yl)methoxy)carbonyl)oxy)butyl) carbamate (0.85 g, 1.524 mmol, 1 eq) gave the desired product as HCl salt (0.753 mg, yield: 100%); MS (m/z): [M+H]+ calcd for C₂₂H₃₁N₇O₄, 458.24; found, 458.1. ¹H-NMR (400 MHz, CD₃OD) δ ppm 8.41 (s, 1H), 7.64 (d, J=3.7 Hz, 1H), 7.01 (d, J=3.4 Hz, 1H), 6.28 (s, 2H), 4.69 (s, 1H), 4.21-3.40 (m, 11H), 2.95 (t, J=6.8 Hz, 1H), 2.56 (br, 1H), 2.05 (br, 1H), 1.87-1.66 (m, 5H), 1.23-1.14 (m, 3H).

### Step 5: Preparation of conjugate of 4-aminobutyl ((4-(((3R,4R)-1-(2-cyanoacetyl)-4-methylpiperidin-3-yl)(methyl)amino)-7H-pyrrolo[2,3-d]pyrimidin-7-yl) methyl) carbonate and HA

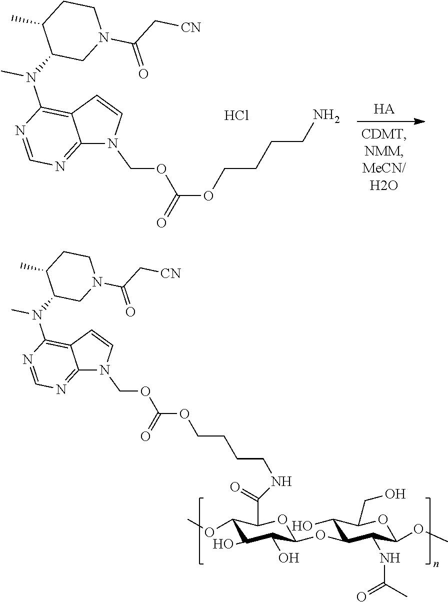

Following Step 3 of Example 1, sodium hyaluronate (MW 50 KDa, 0.161 g, 0.4 mmol, 1 eq) and 4-aminobutyl ((4-(((3R,4R)-1-(2-cyanoacetyl)-4-methylpiperidin-3-yl)(methyl)amino)-7H-pyrrolo[2,3-d]pyrimidin-7-yl) methyl) carbonate hydrochloride (0.2 g, 0.4 mmol, 1 eq) afforded the title compound (0.18 g, yield: 56%, DSR=21%); ¹H-NMR (400 MHz, D₂O) δ ppm 8.2-7.9 (m, 0.21H), 7.3-7.0 (m, 0.21H), 6.8-6.3 (m, 0.21H), 6.1-5.1 (m, 0.42H), 4.45 (d, J=27.3 Hz, 2.84H), 3.8-3.3 (m, 12H), 2.40 (br, 0.21H), 1.97 (s, 3H), 1.65-1.45 (m, 0.94H), 1.25 (t, J=6.9 Hz, 0.21H), 1.05-0.75 (m, 0.7H).

With this step, reaction of TBA hyaluronate (MW 2000 KDa) provided corresponding product (0.172 g, yield: 47.6%, DSR=14%).

### Example 55

### Preparation of conjugate of (4-(((3R,4R)-1-(2-cyanoacetyl)-4-methylpiperidin-3-yl)(methyl)amino)-7H-pyrrolo[2,3-d]pyrimidin-7-yl)methyl(4-aminobutyl)carbamate and HA

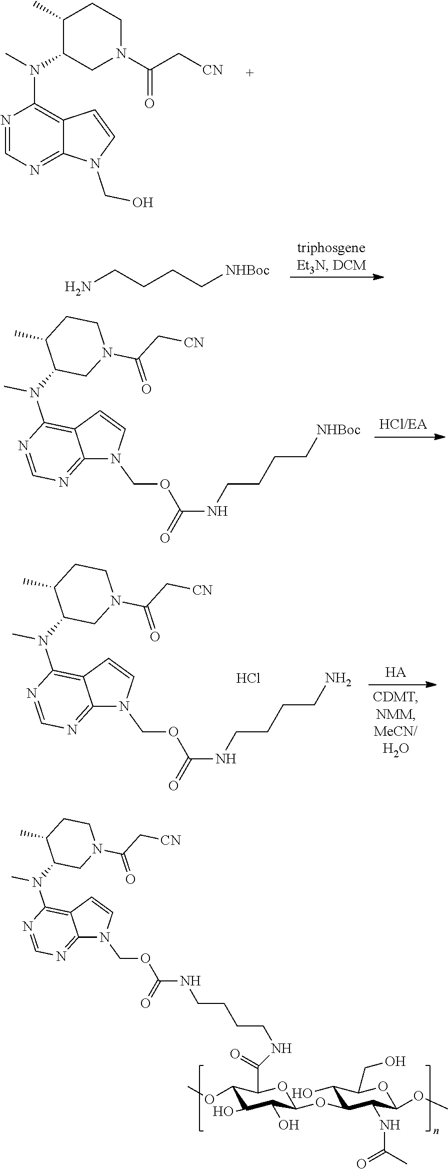

### Step 1: Preparation of tert-butyl ((4-(((3R,4R)-1-(2-cyanoacetyl)-4-methylpiperidin-3-yl)(methy l)amino)-7H-pyrrolo[2,3-d]pyrimidin-7-yl)methyl)butane-1,4-diyldicarbamate

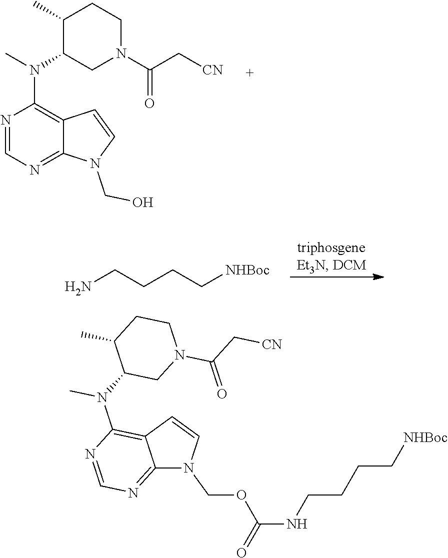

Following Step 1 of Example 1, 3-((3R,4R)-3-((7-(hydroxymethyl)-7H-pyrrolo[2,3-d]pyrimidin-4-yl)(methyl)amino)-4-methylpiperidin-1-yl)-3-oxopropanenitrile (0.6 g, 1.75 mmol, 1 eq) and bis(4-nitrophenyl)carbonate (0.587 g, 1.928 mmol, 1.1 eq) and tert-butyl (4-aminobutyl)carbamate (0.33 g, 1.75 mmol, 1 eq) gave the title product (0.878 g, yield: 90%); m/z (ESI): 557.31[M+H]⁺. MS (m/z): [M+H]+ calcd for C₂₇H₄₀N₈O₅, 557.31; found, 557.2.

### Step 2: Preparation of (4-(((3R,4R)-1-(2-cyanoacetyl)-4-methylpiperidin-3-yl)(methyl)amino)-7H-pyrrolo[2,3-d]pyrimidin-7-yl)methyl(4-aminobutyl)carbamate hydrochloride

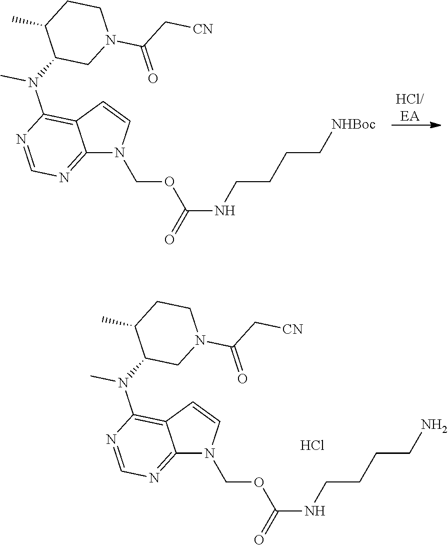 [SMILES: C[C@@H]1CCN(C(=O)CC#N)C[C@@H]1N(C)c1ncnc2c1ccn2COC(=O)NCCCCNC(=O)OC(C)(C)C ; "HCl/EA" ; C[C@@H]1CCN(C(=O)CC#N)C[C@@H]1N(C)c1ncnc2c1ccn2COC(=O)NCCCCN ; Cl]

Following Step 2 of Example 1, tert-butyl ((4-(((3R,4R)-1-(2-cyanoacetyl)-4-methylpiperidin-3-yl)(methyl)amino)-7H-pyrrolo[2,3-d]pyrimidin-7-yl)methyl)butane-1,4-diyldicarbamate (0.8 g, 1.44 mmol, 1 eq) gave the desired product as HCl salt (0.7 g, yield: 100%); MS (m/z): [M+H]+ calcd for C₂₂H₃₂N₈O₃, 457.25; found, 457.2. ¹H-NMR (400 MHz, D₂O) δ ppm 8.33 (t, J=6.7 Hz, 1H), 7.52 (dd, J=17.0, 3.7 Hz, 1H), 6.88 (d, J=9.3 Hz, 1H), 6.17 (d, J=24.8 Hz, 2H), 4.58 (s, 1H), 4.10-3.81 (m, 3H), 3.68-3.16 (m, 5H), 3.16-2.88 (m, 5H), 2.56 (br, 1H), 2.0-1.97 (m, 1H), 1.83-1.72 (m, 1H), 1.67-1.49 (m, 4H), 1.13 (dd, J=14.9, 7.0 Hz, 3H).

### Step 3: Preparation of conjugate of (4-(((3R,4R)-1-(2-cyanoacetyl)-4-methylpiperidin-3-yl)(methyl)amino)-7H-pyrrolo[2,3-d]pyrimidin-7-yl)methyl(4-amino butyl)carbamate and HA

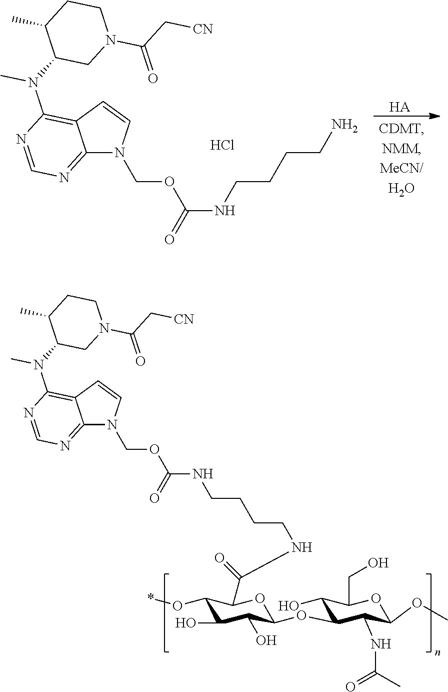

Following Step 3 of Example 1, sodium hyaluronate (MW 50 KDa, 0.161 g, 0.4 mmol, 1 eq) and 4-aminobutyl(4-(((3R,4R)-1-(2-cyanoacetyl)-4-methylpiperidin-3-yl)(methyl)amino)-7H-pyrrolo[2,3-d]pyrimidin-7-yl)methyl(4-aminobutyl)carbamate hydrochloride (0.2 g, 0.4 mmol, 1 eq) afforded the title compound (0.17 g, yield: 50%, DSR=6%); ¹H-NMR (400 MHz, D₂O) δ ppm 8.15-7.95 (m, 0.06H), 7.27 (br, 0.06H), 6.68 (br, 0.06H), 6.1-5.9 (m, 0.12H), 4.40 (d, 2H), 3.93-3.11 (m, 10.8H), 2.37 (br, 0.06H), 2.01-1.79 (s, 3H), 1.6-1.5 (m, 0.36H), 1.0-0.9 (m, 0.18H).

With this step, reaction of sodium hyaluronate (MW 2000 KDa) provided corresponding product (0.150 g, yield: 44.9%, DSR=8%).

### Example 56

### Preparation of conjugate of N-(2-chloroethyl)-4-[[(3R,4R)-1-(2-cyanoacetyl)-4-methyl-3-piperidyl]-methyl-amino]pyrrolo[2,3-d]pyrimidine-7-carboxamide and HA

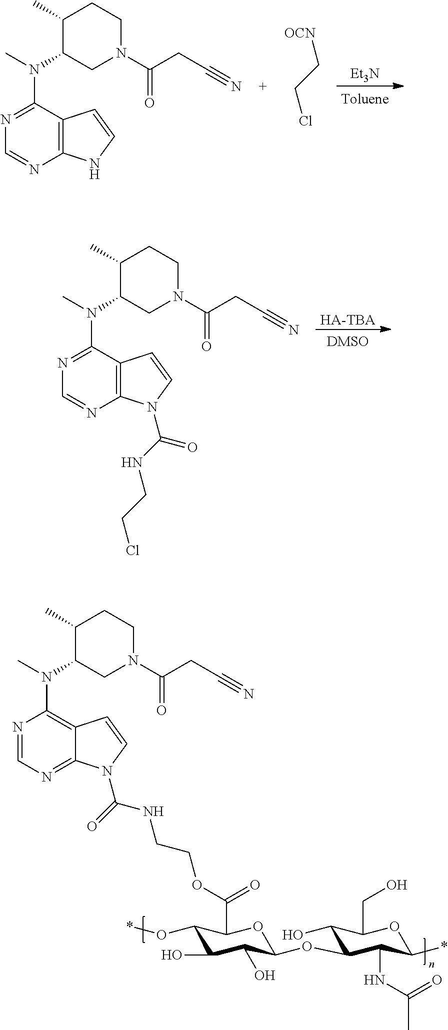

### Step 1: Preparation of N-(2-chloroethyl)-4-[[(3R,4R)-1-(2-cyanoacetyl)-4-methyl-3-piperidyl]-methyl-amino]pyrrolo[2,3-d]pyrimidine-7-carboxamide

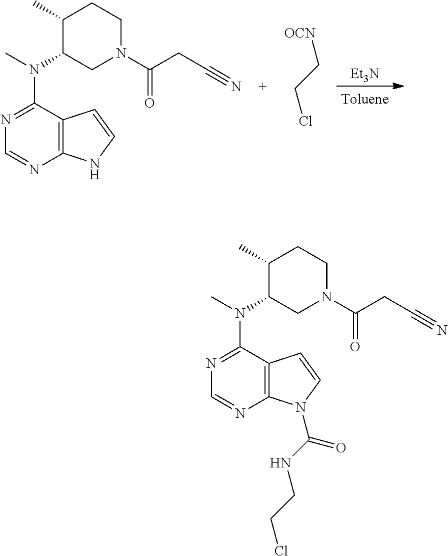

To a stirred mixture of tofacitinib (1.245 g, 4 mmol) in toluene (50 mL) was added triethylamine (0.558 mL, 8 mmol). The reaction mixture was heated to 50° C. 2-chloroethylisocyanate (1688 mg, 16 mmol) was then added to the reaction mixture. The reaction mixture was stirred at this temperature for 4 h. Then the reaction mixture was filtered and then concentrated under reduced pressure. The crude residue was purified by column chromatography (methanol/dichloromethane=1:100˜1:20) to afford the title compound (1.2 g, 71.9%). MS (m/z): [M+H]+ calcd for C₁₉H₂₄C₁N₇O₂, 418.16; found, 418.1.

### Step 2: Preparation of conjugate of N-(2-chloroethyl)-4-[[(3R,4R)-1-(2-cyanoacetyl)-4-methyl-3-piperidyl]-methyl-amino]pyrrolo[2,3-d]pyrimidine-7-carboxamide and HA

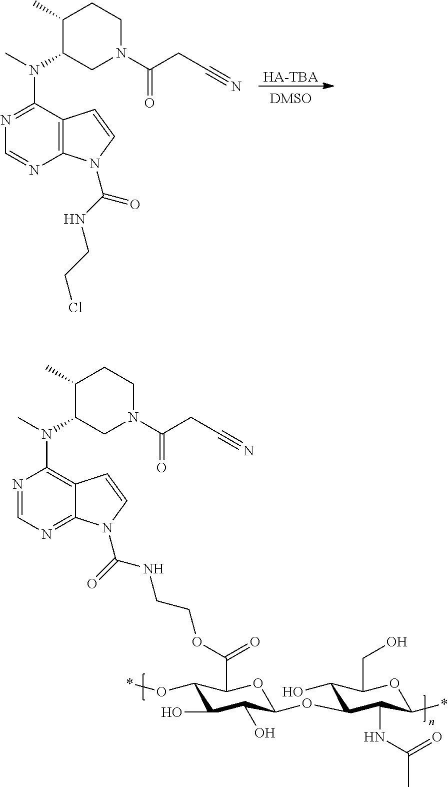

To a stirred mixture of TBA salt of hyaluronic acid (HA-TBA, 331 mg, 0.5 mmol, sodium hyaluronate MW 50 kDa) in anhydrous DMSO (20 mL) was slowly added a solution of N-(2-chloroethyl)-4-[[(3R,4R)-1-(2-cyanoacetyl)-4-methyl-3-piperidyl]-methyl-amino]pyrrolo[2,3-d]pyrimidine-7-carboxamide (209 mg, 0.5 mmol) in anhydrous DMSO (2 mL). The reaction mixture was stirred for 7 days at room temperature and followed by the dropwise addition of acetone (200 mL) while stirring. The mixture was filtered. The filter cake was dissolved in 40 mL of deionized water. Extensive dialysis (3.5 kDa MW cutoff) of the solution against deionized water and lyophilization afforded the title compound. Sodium hyaluronate MW 50 kDa, 0.177 g, Yield: 42.3%, DSR: 10%; ¹H NMR (400 MHz, d-DMSO) δ ppm 8.29-8.17 (m, 0.1H), 7.68-7.54 (m, 0.1H), 6.82-6.68 (m, 0.1H), 4.63-4.28 (m, 2.1H), 4.23-3.17 (m, 11.3H), 2.44-2.33 (m, 0.1H), 2.14-1.58 (m, 3.2H), 1.47-1.36 (m, 0.1H), 1.28-1.17 (m, 0.1H), 1.11-0.89 (m, 0.3H).

### Example 57

### Preparation of conjugate of 2-chloroethyl 4-[[(3R,4R)-1-(2-cyanoacetyl)-4-methyl-3-piperidyl]-methyl-amino]pyrrolo[2,3-d]pyrimidine-7-carboxylate and HA

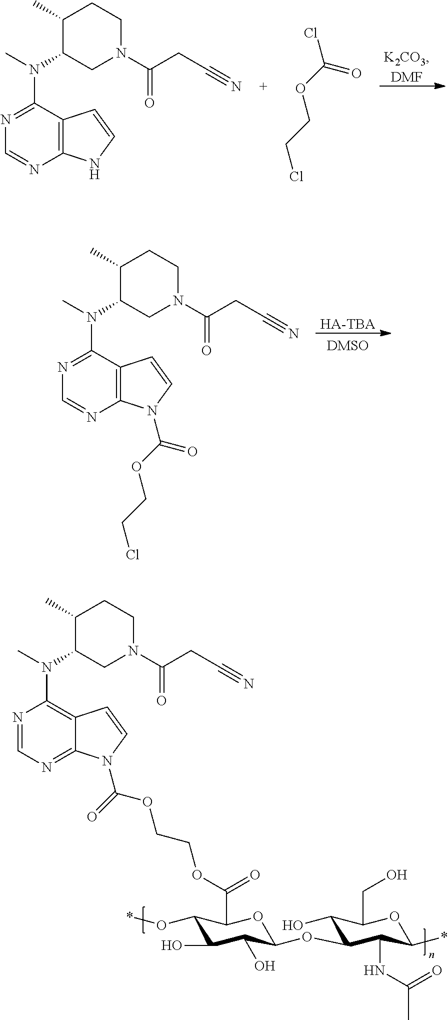

### Step 1: Preparation of 2-chloroethyl 4-[[(3R,4R)-1-(2-cyanoacetyl)-4-methyl-3-piperidyl]-methyl-amino]pyrrolo[2,3-d]pyrimidine-7-carboxylate

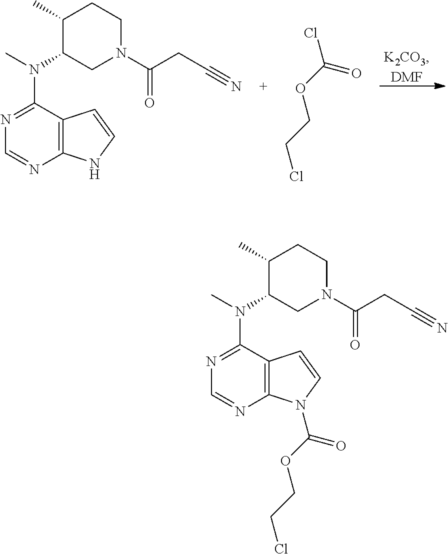

To a stirred mixture of tofacitinib (156 mg, 0.5 mmol) and K₂CO₃ (138 mg, 1 mmol) in anhydrous DMF (2 mL) was added 2-chloroethyl chloroformate (0.1 mL, I mmol). The reaction mixture was stirred for 30 mins at room temperature. The solution was diluted with dichloromethane and washed with water and saturated brine solution. The organic layer was dried over sodium sulfate, filtered and then concentrated under reduced pressure. The crude residue was purified by column chromatography (methanol/dichloromethane==1:100˜1:50) to afford the title compound (0.08 g, 40%). MS (m/z): [M+H]+ calcd for C₁₉H₂₃ClN₆O₃, 419.15; found, 419.3.

### Step 2: Preparation of conjugate of 2-chloroethyl 4-[[(3R,4R)-1-(2-cyanoacetyl)-4-methyl-3-piperidyl]-methyl-amino]pyrrolo[2,3-d]pyrimidine-7-carboxylate and HA

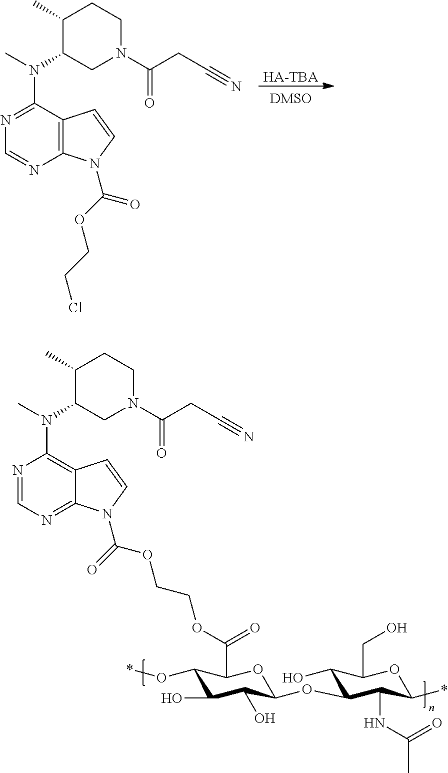

To a stirred mixture of TBA salt of hyaluronic acid (HA-TBA, MW 500 kDa; 331 mg, 0.5 mmol, Sodium hyaluronate MW 50 kDa) in anhydrous DMSO (20 mL) was slowly added a solution of 2-chloroethyl 4-[[(3R,4R)-1-(2-cyanoacetyl)-4-methyl-3-piperidyl]-methyl-amino]pyrrolo[2,3-d]pyrimidine-7-carboxylate (209 mg, 0.5 mmol) in anhydrous DMSO (2 mL). The reaction mixture was stirred for 7 days at room temperature and followed by the dropwise addition of acetone (200 mL) while stirring. The mixture was filtered. The filter cake was dissolved in 40 mL of deionized water. Extensive dialysis (3.5 kDa MW cutoff) of the solution against deionized water and lyophilization afforded the title compound. 0.28 g, Yield: 73.5%, DSR: 25%; ¹H NMR (400 MHz, D₂O) δ ppm 8.21-7.99 (m, 0.25H), 7.57-7.10 (m, 0.25H), 6.83-6.57 (m, 0.25H), 4.54-4.31 (m, 2.25H), 4.09-2.73 (m, 12.75H), 2.39-2.23 (m, 0.25H), 2.02-1.50 (m, 3.75H), 1.32-1.21 (m, 0.25H), 1.00-0.79 (m, 0.75H).

With this step, reaction of HA-TBA (Sodium hyaluronate MW 2000 KDa) provided corresponding product (0.18 g, yield: 47.2%, DSR=20%). ¹H-NMR

### Example 58

### Preparation of conjugate of 3-[(3R,4R)-3-[[7-(2-chloroethoxymethyl) pyrrolo[2,3-d]pyrimidin-4-yl]-methyl-amino]-4-methyl-1-piperidyl]-3-oxo-propanenitrile and HA

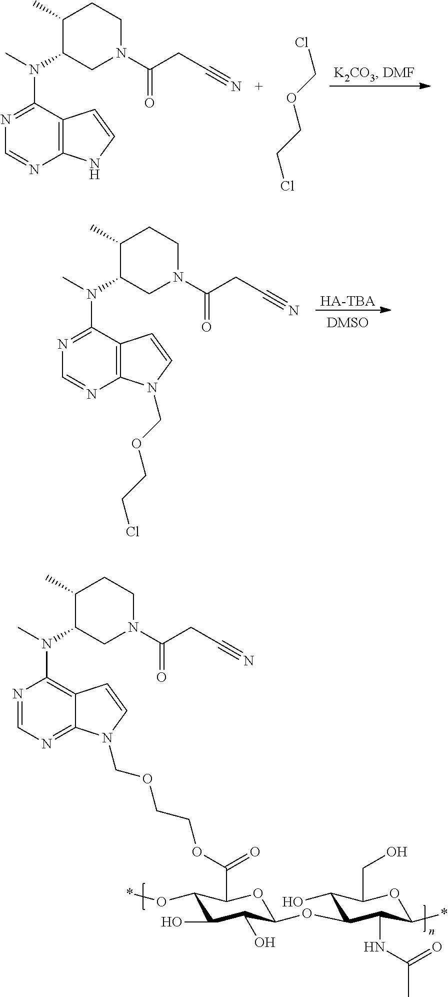

### Step 1: Preparation of 3-[(3R,4R)-3-[[7-(2-chloroethoxymethyl)pyrrolo[2,3-d]pyrimidin-4-yl]-methyl-amino]-4-methyl-1-piperidyl]-3-oxo-propanenitrile

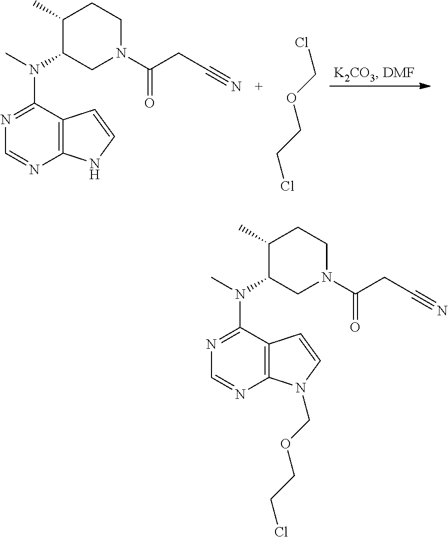

To a stirred mixture of tofacitinib (156 mg, 0.5 mmol) and K₂CO₃ (138 mg, 1 mmol) in anhydrous DMF (2 mL) was added 2-chloromethoxyethylchloride (142 mg, 1.1 mmol). The reaction mixture was stirred for 1 h at room temperature. The solution was diluted with dichloromethane and washed with water and saturated brine solution. The organic layer was dried over sodium sulfate, filtered and then concentrated under reduced pressure. The crude residue was purified by column chromatography (methanol/dichloromethane=1:100˜1:50) to afford the title compound (0.11 g, 55%). MS (m/z): [M+H]+ calcd for C₁₉H₂₅ClN₆O₂, 405.17; found, 405.3.

### Step 2: Preparation of conjugate of 3-[(3R,4R)-3-[[7-(2-chloroethoxymethyl)pyrrolo[2,3-d]pyrimidin-4-yl]-methyl-amino]-4-methyl-1-piperidyl]-3-oxo-propanenitrile and HA

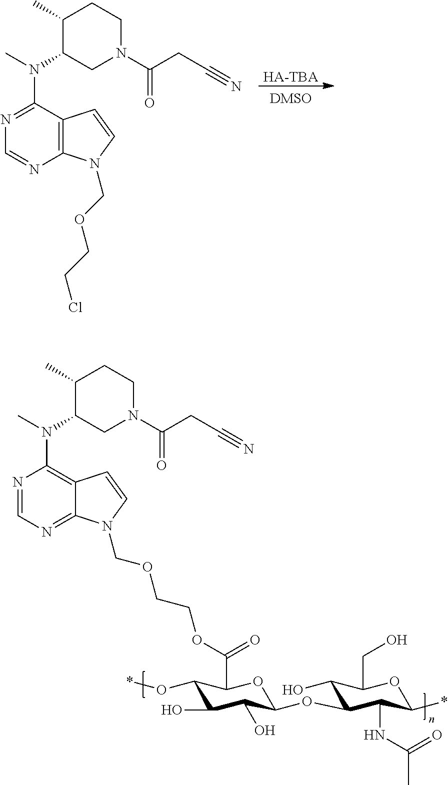

To a stirred mixture of TBA salt of hyaluronic acid (HA-TBA, 331 mg, 0.5 mmol, Sodium hyaluronate MW 50 kDa) in anhydrous DMSO (20 mL) was slowly added a solution of 3-[(3R,4R)-3-[[7-(2-chloroethoxymethyl)pyrrolo[2,3-d]pyrimidin-4-yl]-methyl-amino]-4-methyl-1-piperidyl]-3-oxo-propanenitrile (202 mg, 0.5 mmol) in anhydrous DMSO (2 mL). The reaction mixture was stirred for 4 days at room temperature and followed by the dropwise addition of acetone (200 mL) while stirring. The mixture was filtered. The filter cake was dissolved in 40 mL of deionized water. Extensive dialysis (3.5 kDa MW cutoff) of the solution against deionized water and lyophilization afforded the title compound, 0.28 g, Yield: 74.9%, DSR: 10%; ¹H NMR (400 MHz, d-DMSO) δ ppm 8.28-8.14 (m, 0.1H), 7.67-7.56 (m, 0.1H), 6.82-6.68 (m, 0.1H), 4.66-4.28 (m, 2.1H), 4.23-3.17 (m, 11.3H), 2.44-2.33 (m, 0.1H), 2.19-1.55 (m, 3.2H), 1.47-1.36 (m, 0.1H), 1.28-1.17 (m, 0.1H), 1.11-0.89 (m, 0.3H).

With this step, reaction of H A-TBA (Sodium hyaluronate MW 2000 KDa) provided corresponding product (0.175 g, yield: 46.8%, DSR=35%).

### Example 59

### Preparation of conjugate between HA (Sodium hyaluronate) and methyl (2S)-6-amino-2-[[4-[[4-[[(3R,4R)-1-(2-cyanoacetyl)-4-methyl-3-piperidyl]-methyl-amino]pyrrolo[2,3-d]pyrimidine-7-carbonyl]amino]benzoyl]amino]hexanoate

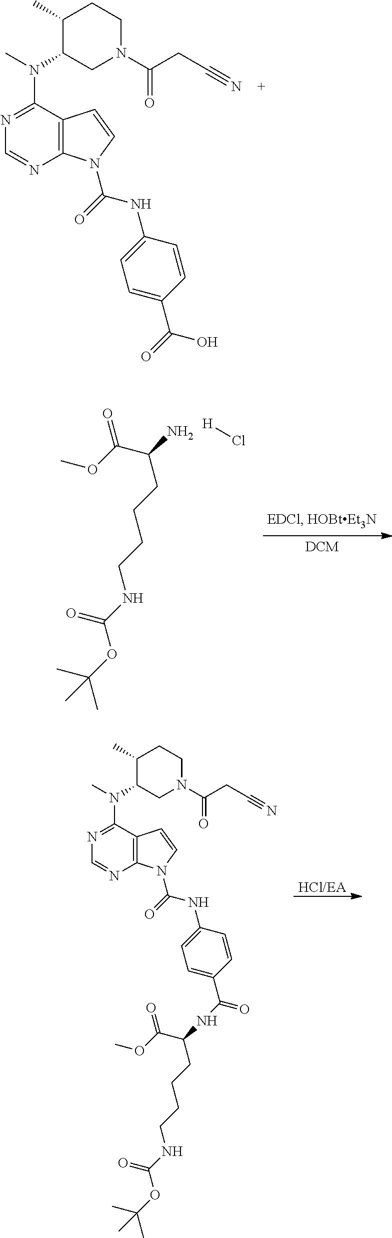

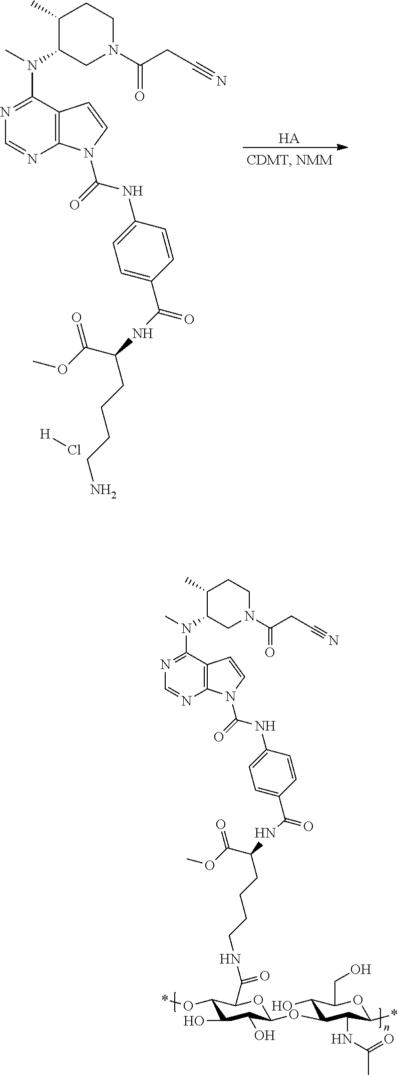

### Step 1: Preparation of methyl (2S)-6-(tert-butoxycarbonylamino)-2-[[4-[[4-[[(3R,4R)-1-(2-cyanoacetyl)-4-methyl-3-piperidyl]-methyl-amino]pyrrolo[2,3-d]pyrimidine-7-carbonyl]amino]benzoyl]amino]hexanoate

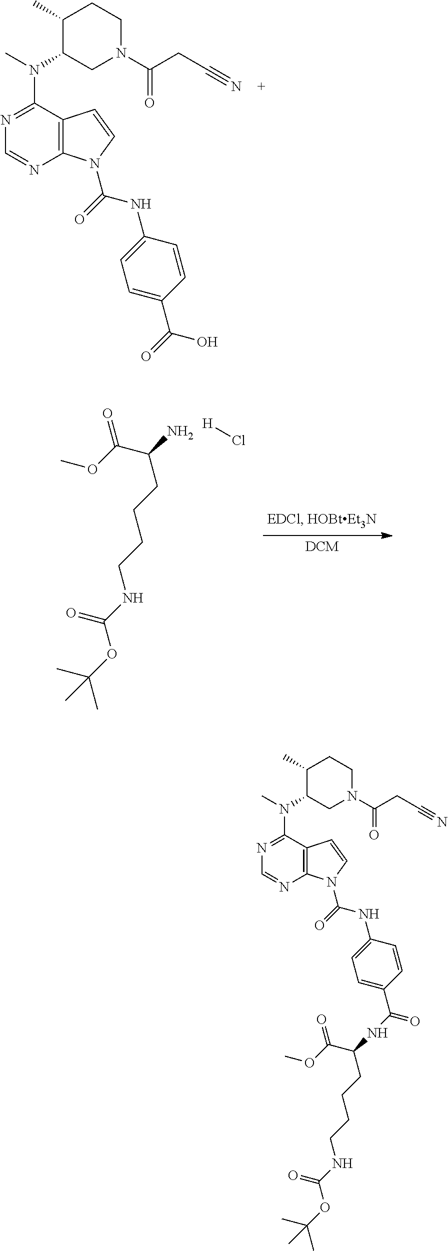

4-[[4-[[(3R,4R)-1-(2-cyanoacetyl)-4-methyl-3-piperidyl]-methyl-amino]pyrrolo[2,3-d]pyrimidine-7-carbonyl]amino]benzoic acid (950 mg, 2 mmol), (S)-Methyl 6-amino-2-((tert-butoxycarbonyl)-amino)hexanoate (890 mg, 3 mmol), 1-ethyl-3-(3-dimethylaminopropyl)carbodiimide (EDCI, 575 mg, 3 mmol) and 1-hydroxybenzotriazole (HOBt, 405 mg, 3 mmol) were dissolved in DCM (40 mL) and triethylamine (1.39 mL, 10 mmol) was added. The reaction mixture was stirred at room temperature for 16 hours. The solution was diluted with DCM and washed with water and saturated brine solution. The organic layer was dried over sodium sulfate, filtered and then concentrated under reduced pressure. The crude residue was purified by column chromatography (Methanol/DCM=1:150-1:80) to afford the title compound (1.1 g, Yield: 76.7%). MS (m/z): [M+H]+ calcd for C₃₆H₄₇N₉O₇, 718.35; found, 718.3. ¹H NMR (400 MHz, CDCl₃) δ ppm 12.29 (d, J=30.5 Hz, 1H), 8.39 (d, J=8.5 Hz, 1H), 7.87 (d, J=8.6 Hz, 2H), 7.78 (dd, J=10.8, 6.4 Hz, 3H), 6.85-6.70 (m, 1H), 6.66 (d, J=3.7 Hz, 1H), 5.11 (d, J=23.2 Hz, 1H), 4.89-4.74 (m, 1H), 4.62 (s, 1H), 4.09 (dd, J=13.0, 3.8 Hz, 1H), 3.95-3.70 (m, 4H), 3.66-3.45 (m, 4H), 3.39 (d, J=14.1 Hz, 3H), 3.13 (d, J=6.0 Hz, 2H), 2.60-2.43 (m, 1H), 2.06-1.91 (m, 2H), 1.91-1.75 (m, 2H), 1.59-1.49 (m, 2H), 1.40 (d, J=19.4 Hz, 11H), 1.27 (d, J=12.0 Hz, 1H), 1.10 (t, J=7.0 Hz, 3H).

### Step 2: Preparation of methyl (2S)-6-amino-2-[[4-[[4-[[(3R,4R)-1-(2-cyanoacetyl)-4-methyl-3-piperidyl]-methyl-amino]pyrrolo[2,3-d]pyrimidine-7-carbonyl]amino]benzoyl]amino]hexanoate hydrochloride

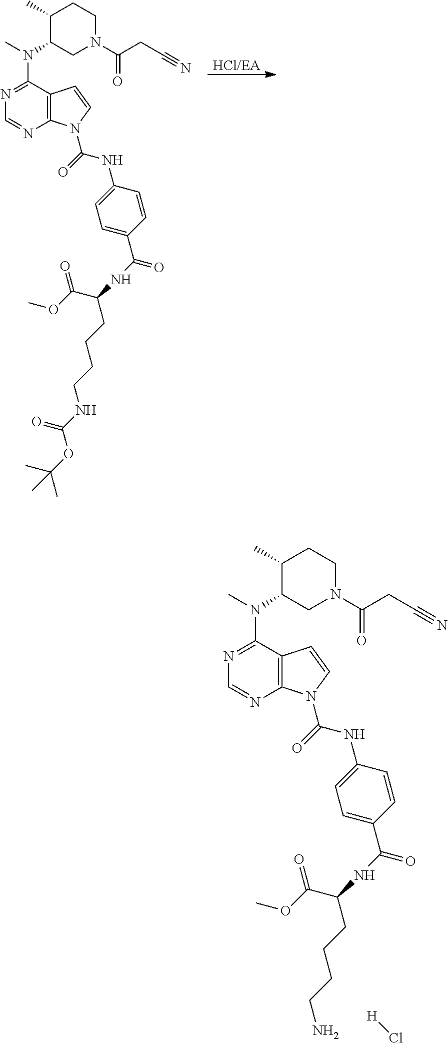

To a stirred solution of methyl (2S)-6-(tert-butoxycarbonylamino)-2-[[4-[[4-[[(3R,4R)-1-(2-cyanoacetyl)-4-methyl-3-piperidyl]-methyl-amino]pyrrolo[2,3-d]pyrimidine-7-carbonyl]amino]benzoyl]amino]hexanoate (1 g, 1.39 mmol) in EtOAc (20 mL) was slowly added 4M HCl in ethyl acetate (commercially available) (4 mL) at ice-bath. The reaction mixture was allowed to cool to room temperature and then was stirred at room temperature for 16 hours. The solution was diluted with EtOAc and concentrated under reduced pressure to afford the title compound as a white solid (0.9 g, yield: 99%) that was used without further purification. MS (m/z): [M+H]+ calcd for C₃₁H₃₉N₉O₅, 618.30; found, 618.1.

### Step 3: Preparation of conjugate between HA (Sodium hyaluronate) and methyl (2S)-6-amino-2-[[4-[[4-[[(3R,4R)-1-(2-cyanoacetyl)-4-methyl-3-piperidyl]-methyl-amino]pyrrolo[2,3-d]pyrimidine-7-carbonyl]amino]benzoyl]amino]hexanoate

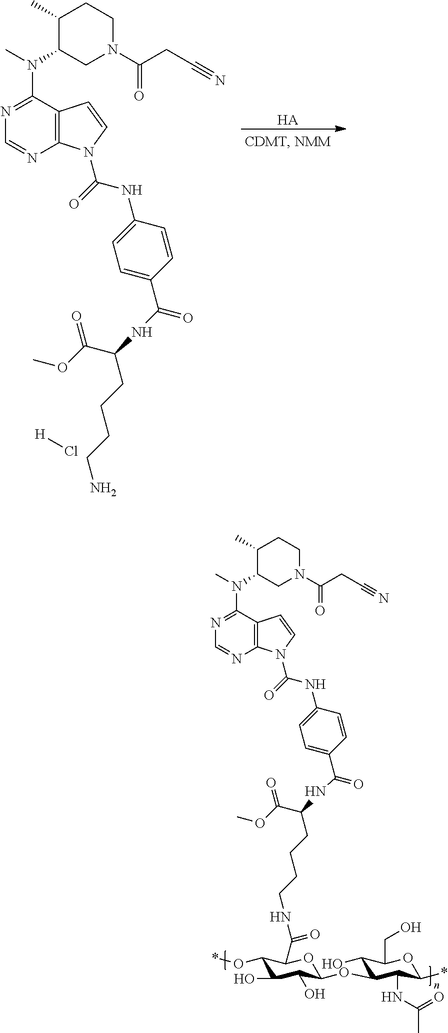

Sodium hyaluronate (117 mg, 0.29 mmol carboxylic acid, MW 50 kDa) was dissolved in 23.4 mL of deionized water in a 100 mL round-bottomed flask followed by the dropwise addition of 16 mL of acetonitrile while stirring. To the solution was added 4-methylmorpholine (NMM, 59 mg, 0.58 mmol), causing the viscosity to increase temporarily. The solution was then cooled to 0° C., and 2-chloro-4,6-dimethoxy-1,3,5-triazine (51 mg, 0.29 mmol) was added and stirred at room temperature for 1 hour. The solution was mixed with methyl (2S)-6-amino-2-[[4-[[4-[[(3R,4R)-1-(2-cyanoacetyl)-4-methyl-3-piperidyl]-methyl-amino]pyrrolo[2,3-d]pyrimidine-7-carbonyl]amino]benzoyl]amino]hexanoate hydrochloride (200 mg, 0.29 mmol) and stirred for 72 hours at room temperature. NaCl (170 mg, 2.9 mmol) was then added to the reaction mixture, which was stirred for 1 hour and followed by the dropwise addition of acetone (290 mL) while stirring. The mixture was filtered. The filter cake was dissolved in 80 mL of deionized water and acetonitrile (V/V=3:1). Extensive dialysis (3.5 kDa Mw cutoff) of the solution against deionized water and lyophilization afforded the title compound. 0.119 g, Yield: 41.9%, DSR: 20%; ¹H NMR (400 MHz, D₂O) δ ppm 8.35-8.23 (m, 0.2H), 7.89-7.40 (m, 1H), 6.83-6.71 (m, 0.2H), 4.61-4.16 (m, 2.2H), 4.13-2.47 (m, 13.4H), 2.09-1.29 (m, 4.6H), 1.23-1.16 (m, 0.2H), 1.06-0.81 (m, 0.6H).

With this step, reaction of Sodium hyaluronate (MW 2000 KDa) provided corresponding product (0.119 g, yield: 41.9%, DSR=21%).

### Example 60

### Preparation of conjugate between HA (Sodium hyaluronate) and N-[3-(4-aminobutylcarbamoyl)phenyl]-4-[[(3R,4R)-1-(2-cyanoacetyl)-4-methyl-3-piperidyl]-methyl-amino]pyrrolo[2,3-d]pyrimidine-7-carboxamide

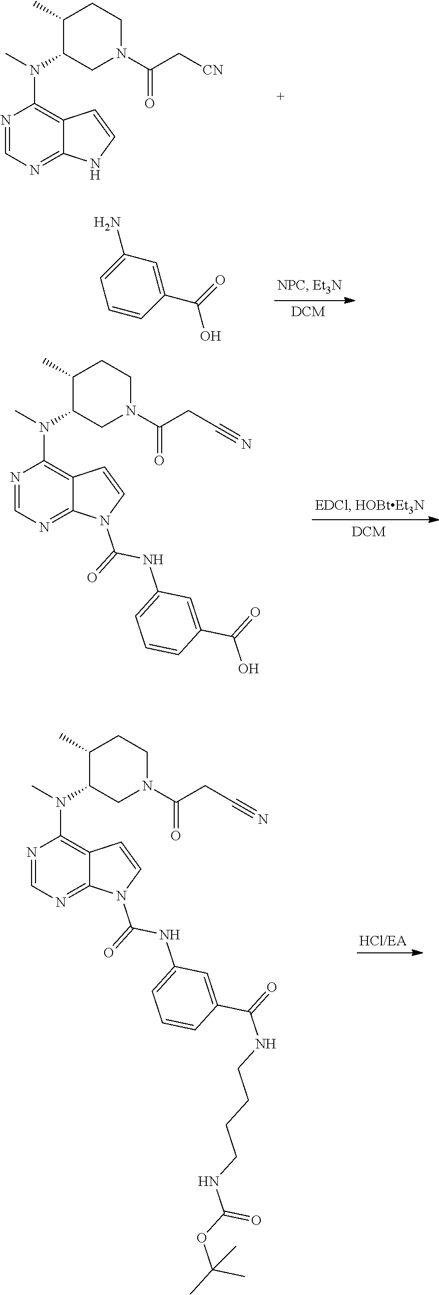 [SMILES: C[C@@H]1CCN(C(=O)CC#N)C[C@@H]1N(C)c1ncnc2[nH]ccc12 ; Nc1cccc(C(=O)O)c1 ; C[C@@H]1CCN(C(=O)CC#N)C[C@@H]1N(C)c1ncnc2c1ccn2C(=O)Nc1cccc(C(=O)O)c1 ; C[C@@H]1CCN(C(=O)CC#N)C[C@@H]1N(C)c1ncnc2c1ccn2C(=O)Nc1cccc(C(=O)NCCCCNC(=O)OC(C)(C)C)c1]

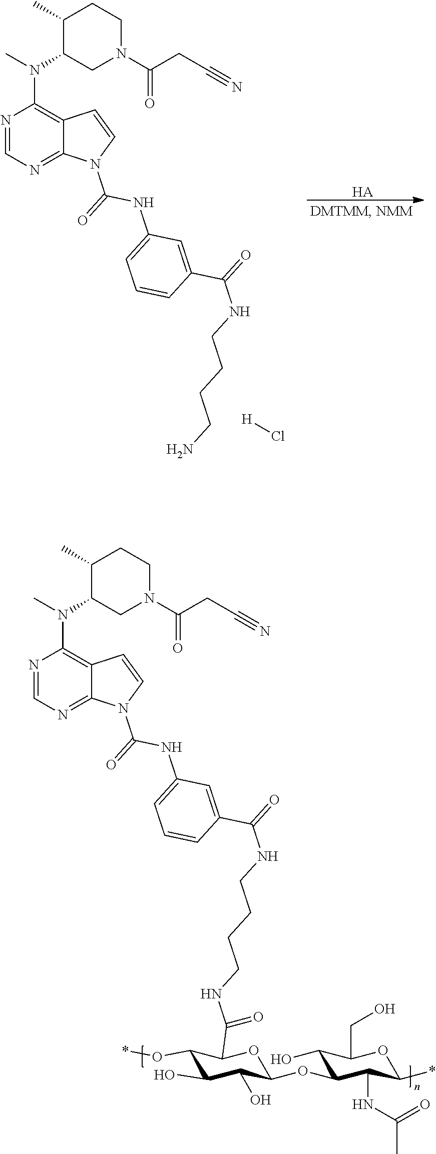

### Step 1: Preparation of 3-[[4-[[(3R,4R)-1-(2-cyanoacetyl)-4-methyl-3-piperidyl]-methyl-amino]pyrrolo[2,3-d]pyrimidine-7-carbonyl]amino]benzoic acid

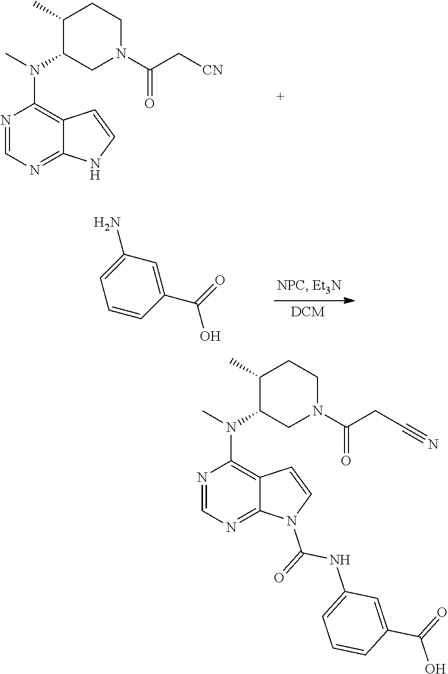

To a stirred mixture of tofacitinib (1248 mg, 4 mmol) and bis(4-nitrophenyl) carbonate (1459 mg, 4.8 mmol) in DCM (40 mL) was added triethylamine (1.12 mL, 8 mmol). The reaction mixture was heated to 45° C. and stirred at this temperature for 5 hours. Then the reaction mixture was cooled to room temperature. 3-aminobenzoic acid (658 mg, 4.8 mmol) was added. The reaction mixture was stirred at 45° C. for 16 hours. The reaction mixture was filtered. The filtrate was washed with water. The organic layer was dried over sodium sulfate, filtered and then concentrated under reduced pressure. The crude residue was purified by column chromatography (DCM:Methanol=20:1) to afford the title compound (1.8 g, Yield: 94.7%). MS (m/z): [M+H]+ calcd for C₂₄H₂₅N₇O₄, 476.19; found, 476.1.

### Step 2: Preparation of tert-butyl N-[4-[[3-[[4-[[(3R,4R)-1-(2-cyanoacetyl)-4-methyl-3-piperidyl]-methyl-amino]pyrrolo[2,3-d]pyrimidine-7-carbonyl]amino]benzoyl]amino]butyl]carbamate

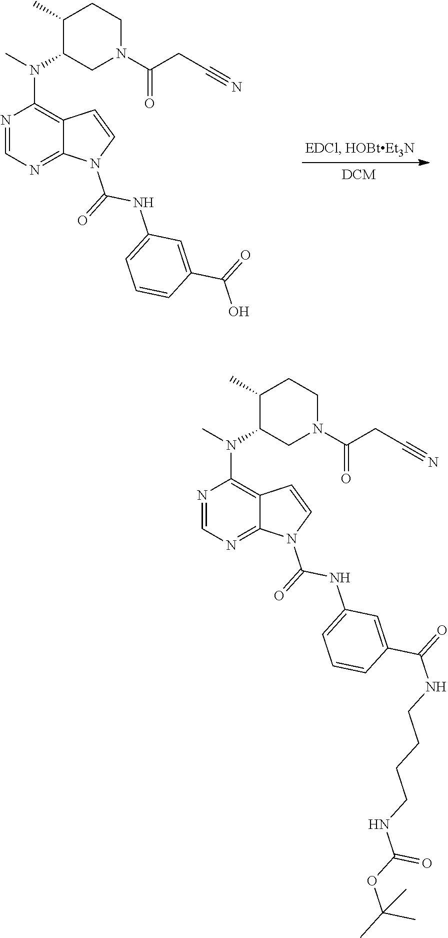 [SMILES: C[C@@H]1CCN(C(=O)CC#N)C[C@@H]1N(C)c1ncnc2c1ccn2C(=O)Nc1cccc(C(=O)O)c1 ; C[C@@H]1CCN(C(=O)CC#N)C[C@@H]1N(C)c1ncnc2c1ccn2C(=O)Nc1cccc(C(=O)NCCCCNC(=O)OC(C)(C)C)c1]

3-[[4-[[(3R,4R)-1-(2-cyanoacetyl)-4-methyl-3-piperidyl]-methyl-amino]pyrrolo[2,3-d]pyrimidine-7-carbonyl]amino]benzoic acid (1188 mg, 2.5 mmol), N-Boc-1,4-butanediamine (705 mg, 3.75 mmol), 1-ethyl-3-(3-dimethylaminopropyl)carbodiimide (EDCI, 719 mg, 3.75 mmol) and 1-hydroxybenzotriazole (HOBt, 507 mg, 3.75 mmol) were dissolved in DCM (40 mL) and triethylamine (1.04 mL, 7.5 mmol) was added. The reaction mixture was stirred at room temperature for 16 hours. The solution was diluted with DCM and washed with water and saturated brine solution. The organic layer was dried over sodium sulfate, filtered and then concentrated under reduced pressure. The crude residue was purified by column chromatography (Methanol/DCM=1:150-1:80) to afford the title compound (1 g, Yield: 62%). MS (m/z): [M+H]+ calcd for C₃₃H₄₃N₉O₅, 646.33; found, 646.3.

### Step 3: Preparation of N-[3-(4-aminobutylcarbamoyl)phenyl]-4-[[(3R,4R)-1-(2-cyanoacetyl)-4-methyl-3-piperidyl]-methyl-amino]pyrrolo[2,3-d]pyrimidine-7-carboxamide hydrochloride

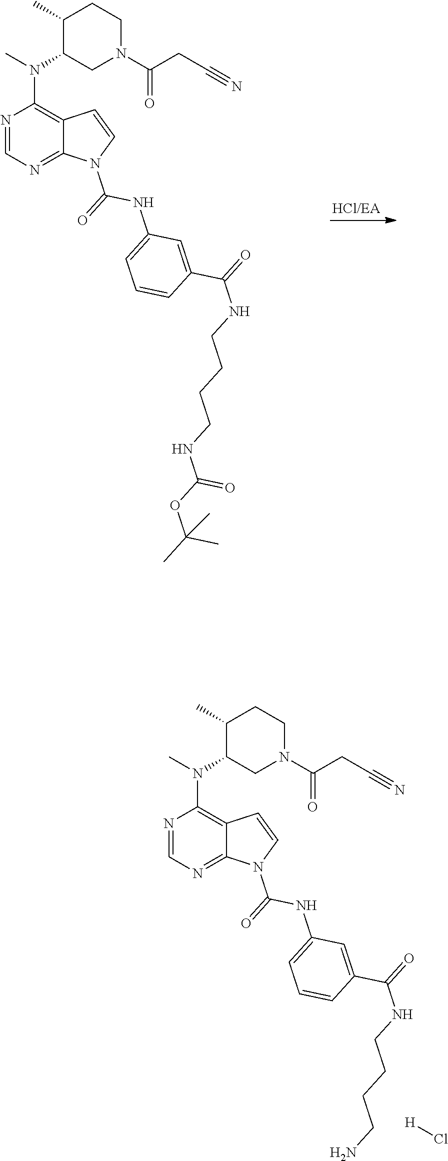

To a stirred solution of tert-butyl N-[4-[[3-[[4-[[(3R,4R)-1-(2-cyanoacetyl)-4-methyl-3-piperidyl]-methyl-amino]pyrrolo[2,3-d]pyrimidine-7-carbonyl]amino]benzoyl]amino]butyl]carbamate (0.6 g, 0.93 mmol) in EtOAc (12 mL) was slowly added 4M HCl in ethyl acetate (commercially available) (2.4 mL) at ice-bath. The reaction mixture was allowed to cool to room temperature and then was stirred at room temperature for 16 hours. The solution was diluted with EtOAc and concentrated under reduced pressure to afford the title compound as a white solid (0.54 g, yield: 99%) that was used without further purification. MS (m/z): [M+H]+ calcd for C₂₈H₃₅N₉O₃, 546.28; found, 546.3.

### Step 4: Preparation of conjugate between HA (Sodium hyaluronate) and N-[3-(4-aminobutylcarbamoyl)phenyl]-4-[[(3R,4R)-1-(2-cyanoacetyl)-4-methyl-3-piperidyl]-methyl-amino]pyrrolo[2,3-d]pyrimidine-7-carboxamide

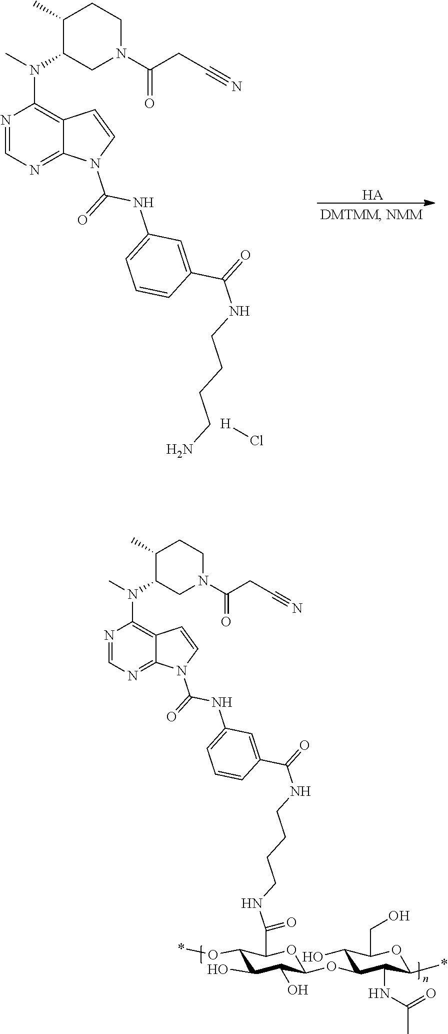

Sodium hyaluronate (186 mg, 0.462 mmol carboxylic acid, MW 50 kDa) was dissolved in 37.2 mL of deionized water in a 100 mL round-bottomed flask followed by the dropwise addition of 24.2 mL of acetonitrile while stirring. To the solution was added N-[3-(4-aminobutylcarbamoyl)phenyl]-4-[[(3R,4R)-1-(2-cyanoacetyl)-4-methyl-3-piperidyl]-methyl-amino]pyrrolo[2,3-d]pyrimidine-7-carboxamide hydrochloride (200 mg, 0.324 mmol) and 4-methylmorpholine (NMM, 32 mg, 0.324 mmol) at room temperature, causing the viscosity to increase temporarily. The reaction mixture was stirred at this temperature for 2 hours. To the solution was added 4-(4,6-dimethoxy-1,3,5-triazin-2-yl)-4-methylmorpholinium chloride (DMTMM, 128 mg, 0.462 mmol) was added and stirred for 72 hours at room temperature. NaCl (270 mg, 4.62 mmol) was then added to the reaction mixture, which was stirred for 1 hour and followed by the dropwise addition of acetone (250 mL) while stirring. The mixture was filtered. The filter cake was dissolved in 80 mL of deionized water and acetonitrile (V/V=3:1). Extensive dialysis (3.5 kDa Mw cutoff) of the solution against deionized water and lyophilization afforded the title compound. 0.209 g, Yield: 49.9%, DSR: 22%; ¹H NMR (400 MHz, D₂O) δ ppm 8.06-7.86 (m, 0.22H), 7.56-7.41 (m, 1.1H), 6.56-6.42 (m, 0.22H), 4.63-4.18 (m, 2.22H), 4.05-2.37 (m, 12.86H), 2.13-1.70 (m, 3.66H), 1.69-1.07 (m, 1.54H).

With this step, reaction of Sodium hyaluronate (MW 2000 KDa) provided corresponding product (0.214 g, yield: 51.1%, DSR=18%).

### Example 61

### Preparation of conjugate between HA (Sodium hyaluronate) and N-[4-(2-aminoethylcarbamoyl)phenyl]-4-[[(3R,4R)-1-(2-cyanoacetyl)-4-methyl-3-piperidyl]-methyl-amino]pyrrolo[2,3-d]pyrimidine-7-carboxamide

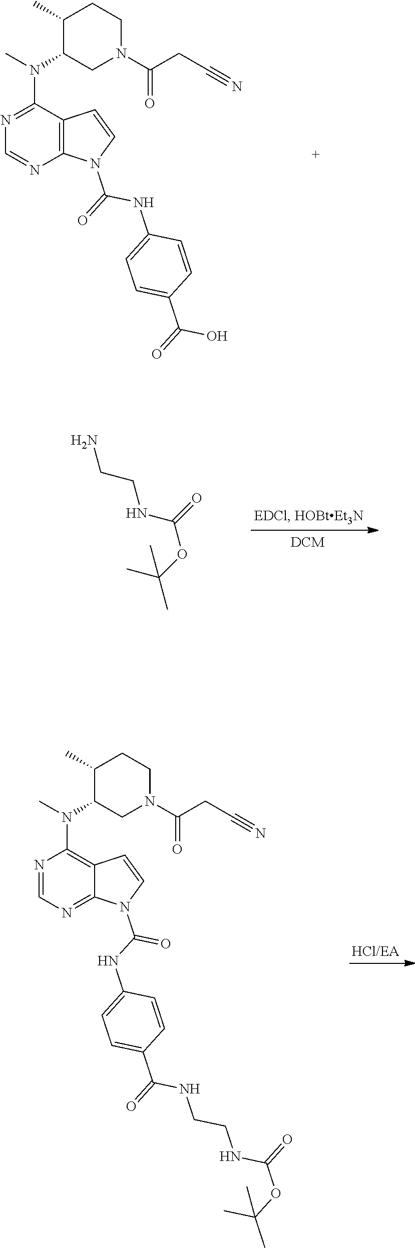 [SMILES: C[C@@H]1CCN(C(=O)CC#N)C[C@@H]1N(C)c1ncnc2c1ccn2C(=O)Nc1ccc(C(=O)O)cc1 ; CC(C)(C)OC(=O)NCCN ; C[C@@H]1CCN(C(=O)CC#N)C[C@@H]1N(C)c1ncnc2c1ccn2C(=O)Nc1ccc(C(=O)NCCNC(=O)OC(C)(C)C)cc1]

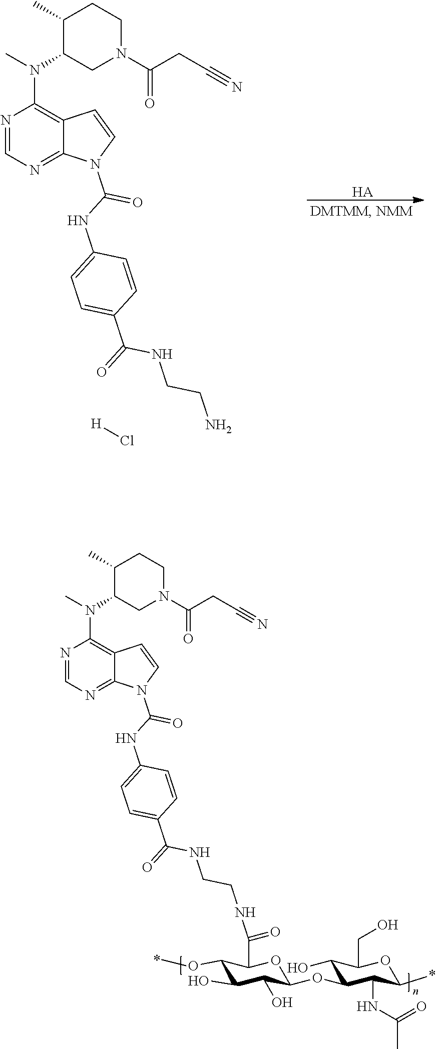

### Step 1: Preparation of tert-butyl N-[2-[[4-[[4-[[(3R,4R)-1-(2-cyanoacetyl)-4-methyl-3-piperidyl]-methyl-amino]pyrrolo[2,3-d]pyrimidine-7-carbonyl]amino]benzoyl]amino]ethyl]carbamate

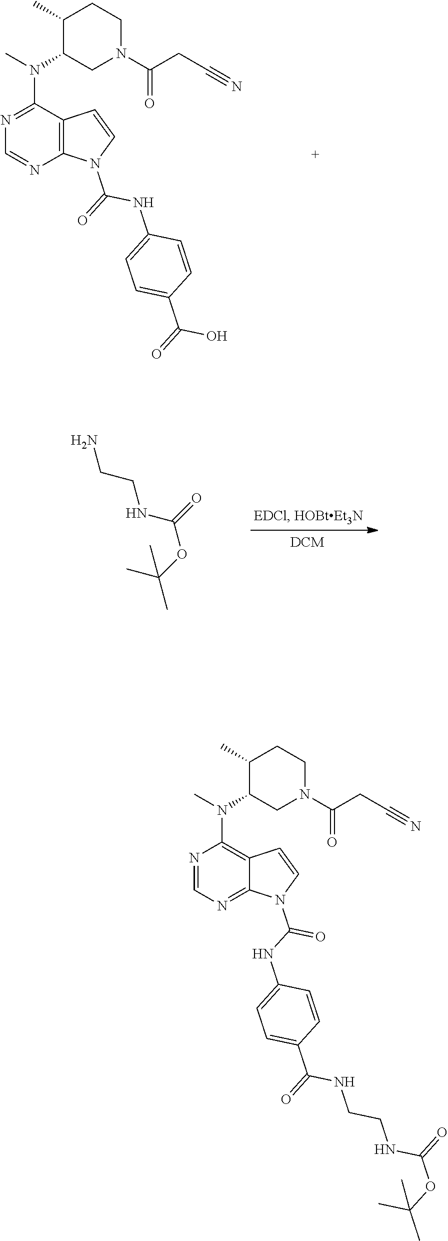 [SMILES: C[C@@H]1CCN(C(=O)CC#N)C[C@@H]1N(C)c1ncnc2c1ccn2C(=O)Nc1ccc(C(=O)O)cc1 ; CC(C)(C)OC(=O)NCCN ; C[C@@H]1CCN(C(=O)CC#N)C[C@@H]1N(C)c1ncnc2c1ccn2C(=O)Nc1ccc(C(=O)NCCNC(=O)OC(C)(C)C)cc1]

4-[[4-[[(3R,4R)-1-(2-cyanoacetyl)-4-methyl-3-piperidyl]-methyl-amino]pyrrolo[2,3-d]pyrimidine-7-carbonyl]amino]benzoic acid (1425 mg, 3 mmol), N-Boc-ethylenediamine (721 mg, 4.5 mmol), 1-ethyl-3-(3-dimethylaminopropyl)carbodiimide (EDCI, 863 mg, 4.5 mmol) and 1-hydroxybenzotriazole (HOBt, 607.5 mg, 4.5 mmol) were dissolved in DCM (40 mL) and triethylamine (1.25 mL, 9 mmol) was added. The reaction mixture was stirred at room temperature for 16 hours. The solution was diluted with DCM and washed with water and saturated brine solution. The organic layer was dried over sodium sulfate, filtered and then concentrated under reduced pressure. The crude residue was purified by column chromatography (Methanol/DCM=1:150˜1:80) to afford the title compound (1.23 g, Yield: 66.4%). MS (m/z): [M+H]+ calcd for C₃₁H₃₉N₉O₅, 618.30; found, 618.2. ¹H NMR (400 MHz, CDCl₃) δ ppm 12.26 (d, J=23.3 Hz, 1H), 8.38 (d, J=9.3 Hz, 1H), 7.81 (dd, J=39.5, 8.6 Hz, 5H), 7.24 (s, 1H), 6.65 (d, J=4.0 Hz, 1H), 5.11 (d, J=33.0 Hz, 2H), 4.13-3.74 (m, 2H), 3.68-3.11 (m, 11H), 2.62-2.45 (m, 1H), 2.08-1.72 (m, 2H), 1.44 (s, 9H), 1.10 (t, J=8.6 Hz, 3H).

### Step 2: Preparation of N-[4-(2-aminoethylcarbamoyl)phenyl]-4-[[(3R,4R)-1-(2-cyanoacetyl)-4-methyl-3-piperidyl]-methyl-amino]pyrrolo[2,3-d]pyrimidine-7-carboxamide hydrochloride

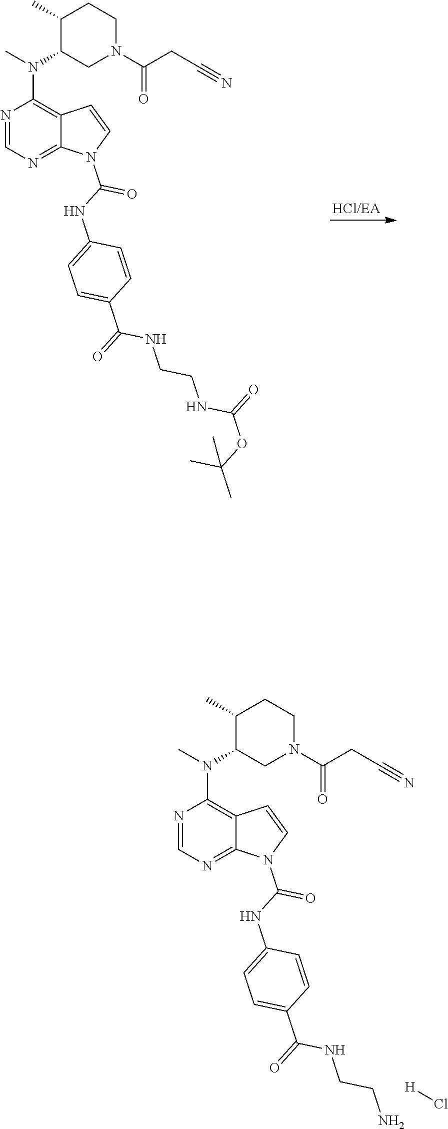

To a stirred solution of tert-butyl N-[2-[[4-[[4-[[(3R,4R)-1-(2-cyanoacetyl)-4-methyl-3-piperidyl]-methyl-amino]pyrrolo[2,3-d]pyrimidine-7-carbonyl]amino]benzoyl]amino]ethyl]carbamate (0.6 g, 0.97 mmol) in EtOAc (6 mL) was slowly added 4M HCl in ethyl acetate (commercially available) (2.4 mL) at ice-bath. The reaction mixture was allowed to cool to room temperature and then was stirred at room temperature for 16 hours. The solution was diluted with EtOAc and concentrated under reduced pressure to afford the title compound as a white solid (0.5 g, yield: 99%) that was used without further purification. MS (m/z): [M+H]+ calcd for C₂₆H₃₁N₉O₃, 518.25; found, 518.2.

### Step 3: Preparation of conjugate between HA (Sodium hyaluronate) and N-[4-(2-aminoethylcarbamoyl)phenyl]-4-[[(3R,4R)-1-(2-cyanoacetyl)-4-methyl-3-piperidyl]-methyl-amino]pyrrolo[2,3-d]pyrimidine-7-carboxamide

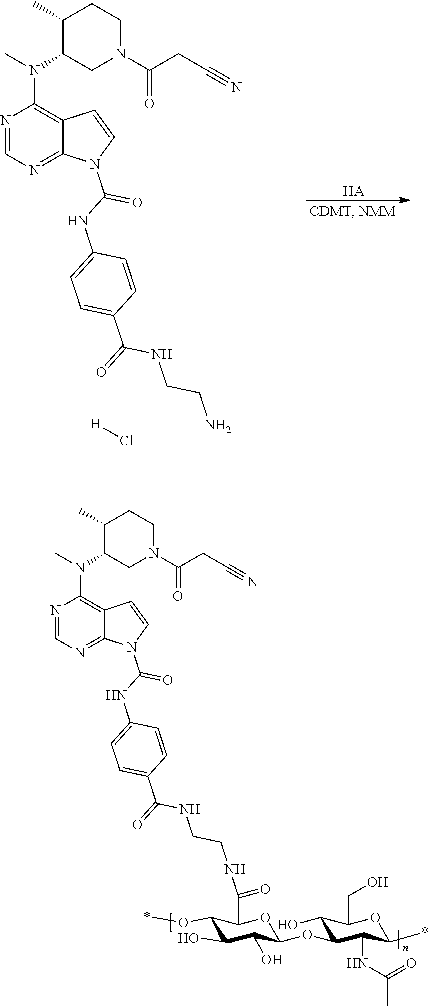

Sodium hyaluronate (208 mg, 0.516 mmol carboxylic acid, MW 50 kDa) was dissolved in 41.6 mL of deionized water in a 100 mL round-bottomed flask followed by the dropwise addition of 27 mL of acetonitrile while stirring. To the solution was added N-[4-(2-aminoethylcarbamoyl)phenyl]-4-[[(3R,4R)-1-(2-cyanoacetyl)-4-methyl-3-piperidyl]-methyl-amino]pyrrolo[2,3-d]pyrimidine-7-carboxamide hydrochloride (200 mg, 0.361 mmol) and 4-methylmorpholine (NMM, 36.5 mg, 0.361 mmol) at room temperature, causing the viscosity to increase temporarily. The reaction mixture was stirred at this temperature for 2 hours. To the solution was added 4-(4,6-dimethoxy-1,3,5-triazin-2-yl)-4-methylmorpholinium chloride (DMTMM, 142.8 mg, 0.516 mmol) was added and stirred for 72 hours at room temperature. NaCl (303 mg, 5.16 mmol) was then added to the reaction mixture, which was stirred for 1 hour and followed by the dropwise addition of acetone (250 mL) while stirring. The mixture was filtered. The filter cake was dissolved in 80 mL of deionized water and acetonitrile (V/V=3:1). Extensive dialysis (3.5 kDa Mw cutoff) of the solution against deionized water and lyophilization afforded the title compound. 0.25 g, Yield: 55.1%, DSR: 22%; ¹H NMR (400 MHz, D₂O) δ ppm 8.36-8.18 (m, 0.22H), 7.92-6.84 (m, 1.1H), 6.68-6.55 (m, 0.22H), 4.61-4.22 (m, 2.22H), 4.13-2.65 (m, 12.86H), 2.24-1.52 (m, 3.66H), 1.30-1.09 (m, 0.66H).

With this step, reaction of Sodium hyaluronate (MW 2000 KDa) provided corresponding product (0.24 g, yield: 52.9%, DSR=26%).

### Example 62

### Preparation of conjugate between HA (Sodium hyaluronate) and methyl (2S)-6-amino-2-[[trans-4-[methyl-[4-(methylsulfamoylmethyl)cyclohexyl]amino]pyrrolo[2,3d]pyrimidine-7-carbonyl]amino]hexanoate

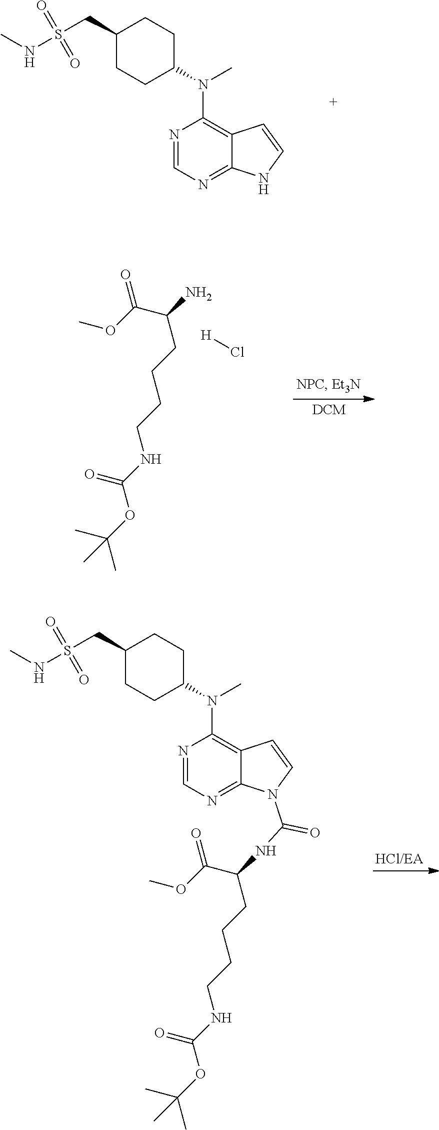

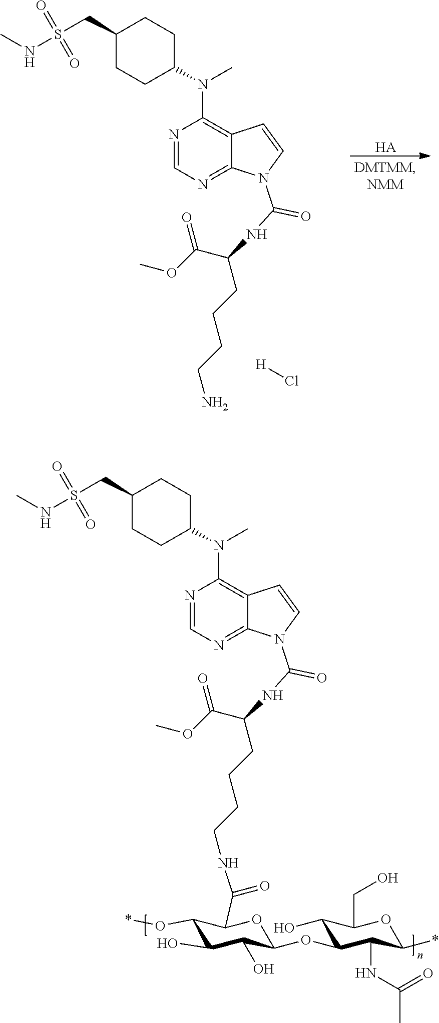

### Step 1: Preparation of methyl (2S)-6-(tert-butoxycarbonylamino)-2-[[trans-4-[methyl-[4-(methylsulfamoylmethyl)cyclohexyl]amino]pyrrolo[2,3-d]pyrimidine-7-carbonyl]amino]hexanoate

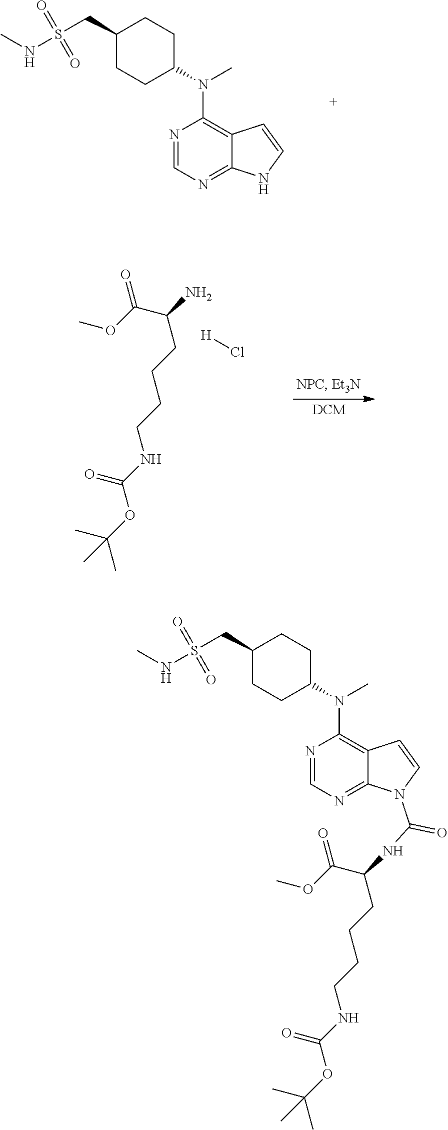

To a stirred mixture of trans-N-methyl-1-[4-[methyl(7H-pyrrolo[2,3-d]pyrimidin-4-yl)amino]cyclohexyl]methanesulfonamide (1012 mg, 3 mmol) and bis(4-nitrophenyl) carbonate (1094 mg, 3.6 mmol) in DCM (40 mL) was added triethylamine (1.25 mL, 9 mmol). The reaction mixture was heated to 45° C. and stirred at this temperature for 5 hours. Then the reaction mixture was cooled to room temperature. (S)-Methyl 6-amino-2-((tert-butoxycarbonyl)-amino)hexanoate (1069 mg, 3.6 mmol) was added. The reaction mixture was stirred at 45° C. for 16 hours. The reaction mixture was filtered. The filtrate was washed with water. The organic layer was dried over sodium sulfate, filtered and then concentrated under reduced pressure. The crude residue was purified by column chromatography (DCM:Methanol=100:1) to afford the title compound (0.66 g, Yield: 35.3%). MS (m/z): [M+H]+ calcd for C₂₈H₄₅N₇O₇S, 624.31; found, 624.2. ¹H NMR (400 MHz, CDCl₃) δ ppm 10.38 (d, J=7.6 Hz, 1H), 8.36 (s, 1H), 7.67 (d, J=4.1 Hz, 1H), 6.59 (d, J=4.0 Hz, 1H), 4.78-4.69 (m, 2H), 4.58 (s, 1H), 4.22-4.05 (m, 1H), 3.81 (s, 3H), 3.24 (s, 3H), 3.14 (d, J=5.4 Hz, 2H), 3.01 (dd, J=15.2, 5.9 Hz, 2H), 2.86 (d, J=5.3 Hz, 3H), 2.21 (d, J=11.6 Hz, 2H), 2.07-1.87 (m, 5H), 1.71 (dd, J=23.8, 11.5 Hz, 2H), 1.59-1.48 (m, 4H), 1.47-1.32 (m, 11H).

### Step 2: Preparation of methyl (2S)-6-amino-2-[[trans-4-[methyl-[4-(methylsulfamoylmethyl)cyclohexyl]amino]pyrrolo[2,3-d]pyrimidine-7-carbonyl]amino]hexanoate hydrochloride

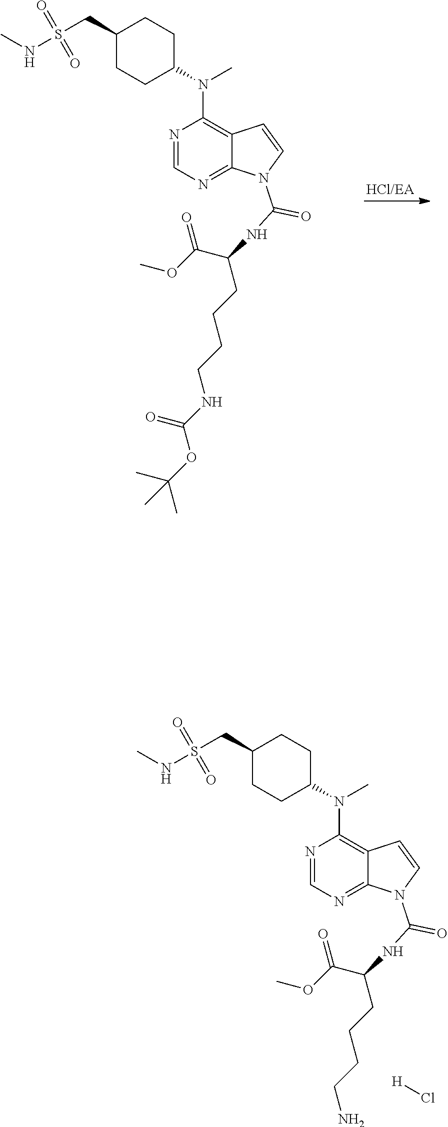

To a stirred solution of methyl (2S)-6-(tert-butoxycarbonylamino)-2-[[trans-4-[methyl-[4-(methylsulfamoylmethyl)cyclohexyl]amino]pyrrolo[2,3-d]pyrimidine-7-carbonyl]amino]hexanoate (0.61 g, 0.97 mmol) in EtOAc (12 mL) was slowly added 4M HCl in ethyl acetate (commercially available) (2.4 mL) at ice-bath. The reaction mixture was allowed to cool to 0° C. and then was stirred at room temperature for 16 hours. The solution was diluted with EtOAc and concentrated under reduced pressure to afford the title compound as a white solid (0.54 g, Yield: 99%) that was used without further purification. MS (m/z): [M+H]+ calcd for C₂₃H₃₇N₇O₅S, 524.25; found, 524.2.

### Step 3: Preparation of conjugate between HA (Sodium hyaluronate) and methyl (2S)-6-amino-2-[[trans-4-[methyl-[4-(methylsulfamoylmethyl)cyclohexyl]amino]pyrrolo[2,3-d]pyrimidine-7-carbonyl]amino]hexanoate

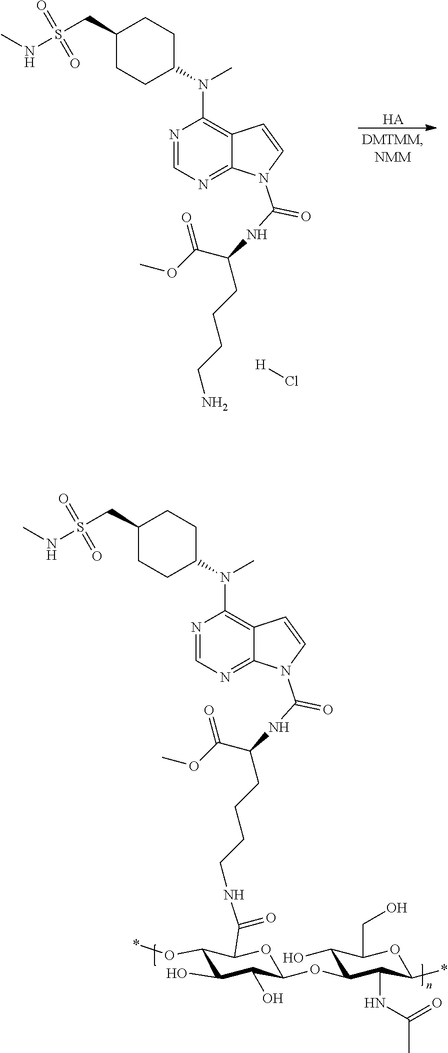

Sodium hyaluronate (101.6 mg, 0.252 mmol carboxylic acid, MW 50 kDa) was dissolved in 21 mL of deionized water in a 100 mL round-bottomed flask followed by the dropwise addition of 13.2 mL of acetonitrile while stirring. To the solution was added 4-methylmorpholine (NMM, 51 mg, 0.504 mmol), causing the viscosity to increase temporarily. The solution was then cooled to 0° C., and 2-chloro-4,6-dimethoxy-1,3,5-triazine (44.3 mg, 0.252 mmol) was added and stirred at room temperature for 1 hour. The solution was mixed with methyl (2S)-6-amino-2-[[trans-4-[methyl-[4-(methylsulfamoylmethyl)cyclohexyl]amino]pyrrolo[2,3-d]pyrimidine-7-carbonyl]amino]hexanoate hydrochloride (150 mg, 0.252 mmol) and stirred for 72 hours at room temperature. NaCl (148 mg, 2.53 mmol) was then added to the reaction mixture, which was stirred for 1 hour and followed by the dropwise addition of acetone (200 mL) while stirring. The mixture was filtered. The filter cake was dissolved in 80 mL of deionized water and acetonitrile (V/V=3:1). NaCl (148 mg, 2.53 mmol) was then added to the reaction mixture, which was stirred for 1 hour and followed by the dropwise addition of acetone (200 mL) while stirring. The mixture was filtered. The filter cake was collected, washed with acetone, and dried in vacuo to give the title compound as a white solid. 0.118 g, Yield: 52.9%, DSR: 16%; ¹H NMR (400 MHz, D₂O) δ ppm 8.29-8.17 (m, 0.16H), 7.66-7.54 (m, 0.16H), 6.84-6.70 (m, 0.16H), 4.59-4.15 (m, 2.16H), 4.11-2.37 (m, 12.24H), 2.11-1.73 (m, 4.44H), 1.71-1.11 (m, 0.96H).

With this step, reaction of Sodium hyaluronate (MW 2000 KDa) provided corresponding product (0.105 g, yield: 47.1%, DSR=20%).

### Example 63

### Preparation of conjugate between HA (Sodium hyaluronate) and 1-[4-[[7-(hydrazinecarbonyl)pyrrolo[2,3-d]pyrimidin-4-yl]-methyl-amino]cyclohexyl]-trans-N-methyl-methanesulfonamide

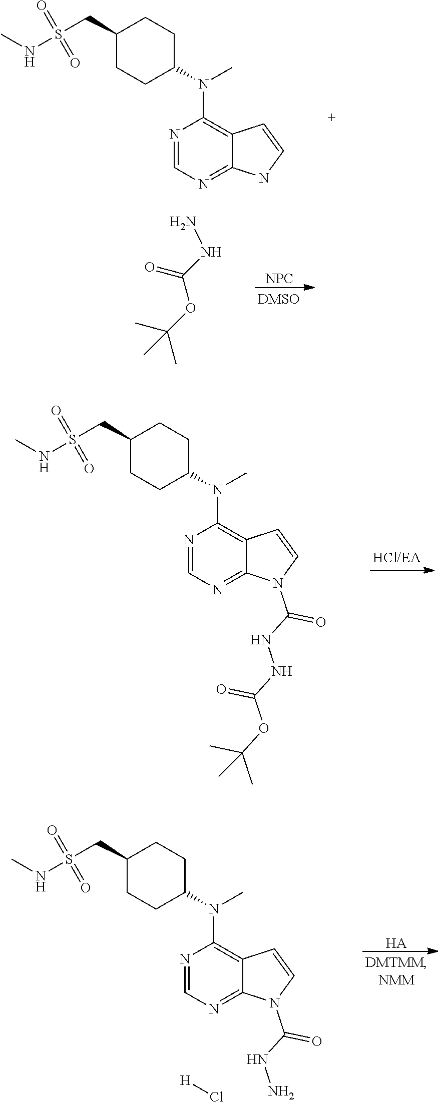

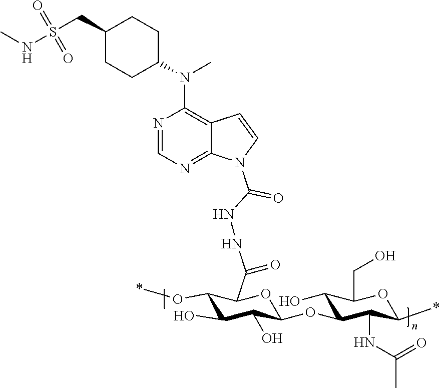

### Step 1: Preparation of tert-butyl N-[[trans-4-[methyl-[4-(methylsulfamoylmethyl)cyclohexyl]amino]pyrrolo[2,3-d]pyrimidine-7-carbonyl]amino]carbamate

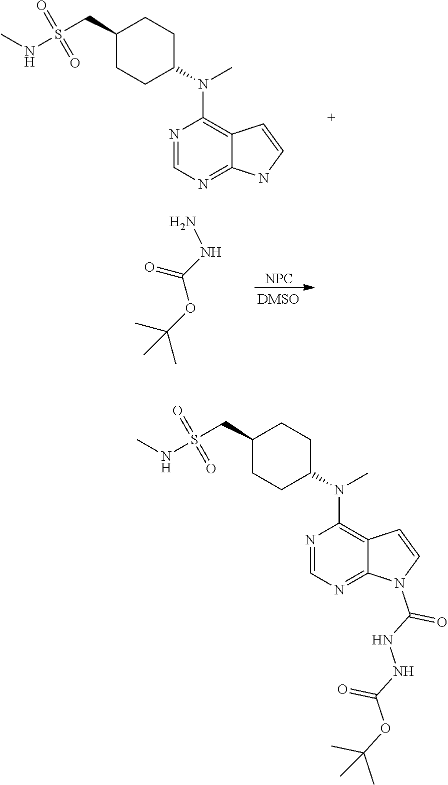

A mixture of Trans-N-methyl-1-[4-[methyl(7H-pyrrolo[2,3-d]pyrimidin-4-yl)amino]cyclohexyl]methanesulfonamide (1350 mg, 4 mmol) and bis(4-nitrophenyl) carbonate (1337.6 mg, 4.4 mmol) in DMSO (14 mL) was stirred at room temperature for 4 hours. Then tert-Butyl hydrazinecarboxylate (634.4 mg, 4.8 mmol) was added. The reaction mixture was stirred at room temperature for 16 hours. The reaction mixture was poured into saturated Na₂CO₃ solution. The mixture was filtered. The filter cake was washed with saturated NaHCO₃ solution and water. Then the filter cake was dissolved in DCM and washed with water. The organic layer was dried over sodium sulfate, filtered and then concentrated under reduced pressure to afford the title compound (1.67 g, Yield: 84.3%). MS (m/z): [M+H]+ calcd for C₂₁H₃₃N₇O₅S, 496.22; found, 496.1. ¹H NMR (400 MHz, CDCl₃) δ ppm 11.38 (s, 1H), 8.30 (s, 1H), 7.63 (d, J=4.1 Hz, 1H), 6.56 (dd, J=25.8, 3.5 Hz, 2H), 4.75 (s, 1H), 4.36 (q, J=5.1 Hz, 1H), 3.22 (d, J=11.1 Hz, 3H), 2.97 (d, J=6.3 Hz, 2H), 2.83 (d, J=5.3 Hz, 3H), 2.20 (t, J=14.4 Hz, 2H), 2.04-1.94 (m, 1H), 1.87 (d, J=10.8 Hz, 2H), 1.74-1.62 (m, 2H), 1.51 (s, 9H), 1.43-1.32 (m, 2H).

### Step 2: Preparation of 1-[4-[[7-(hydrazinecarbonyl)pyrrolo[2,3-d]pyrimidin-4-yl]-methyl-amino]cyclohexyl]-trans-N-methyl-methanesulfonamide hydrochloride

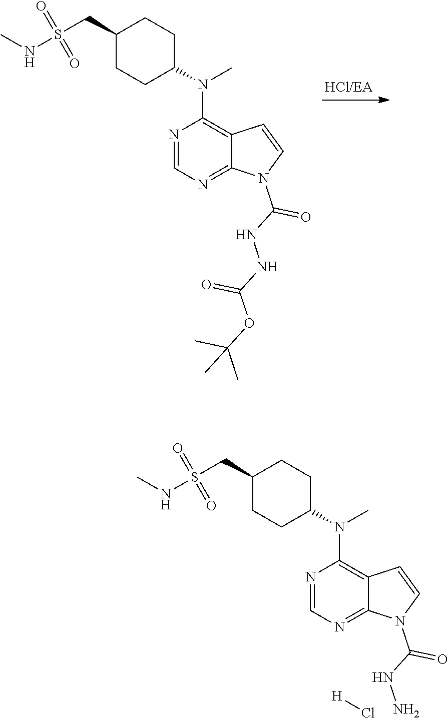

To a stirred solution of tert-butyl N-[[trans-4-[methyl-[4-(methylsulfamoylmethyl)cyclohexyl]amino]pyrrolo[2,3-d]pyrimidine-7-carbonyl]amino]carbamate (0.6 g, 1.21 mmol) in EtOAc (12 mL) was slowly added 4M HCl in ethyl acetate (commercially available) (2.4 mL) at ice-bath. The reaction mixture was allowed to cool to room temperature and then was stirred at room temperature for 16 hours. The solution was diluted with EtOAc and concentrated under reduced pressure to afford the title compound as a white solid (0.52 g, Yield: 99%) that was used without further purification. MS (m/z): [M+H]+ calcd for C₁₆H₂₅N₇O₃S, 396.17; found, 396.1.

### Step 3: Preparation of conjugate between HA (Sodium hyaluronate) and 1-[4-[[7-(hydrazinecarbonyl)pyrrolo[2,3-d]pyrimidin-4-yl]-methyl-amino]cyclohexyl]-trans-N-methyl-methanesulfonamide

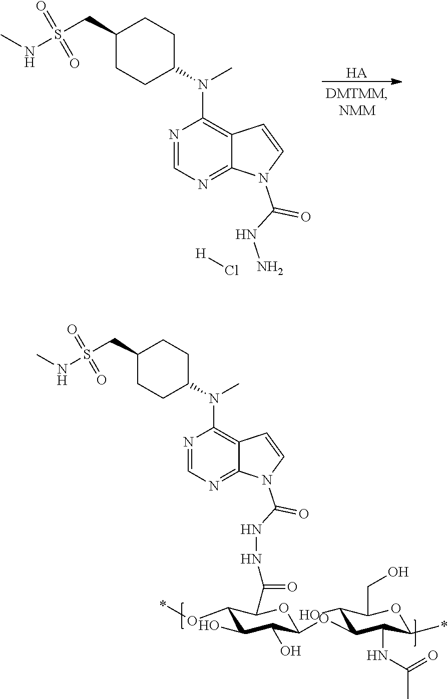

Sodium hyaluronate (246 mg, 0.61 mmol carboxylic acid, MW 50 kDa) was dissolved in 49.2 mL of deionized water in a 100 mL round-bottomed flask followed by the dropwise addition of 32 mL of acetonitrile while stirring. To the solution was added 4-methylmorpholine (NMM, 43 mg, 0.427 mmol), causing the viscosity to increase temporarily. To the solution was added 1-[4-[[7-(hydrazinecarbonyl)pyrrolo[2,3-d]pyrimidin-4-yl]-methyl-amino]cyclohexyl]-trans-N-methyl-methanesulfonamide hydrochloride (200 mg, 0.427 mmol) and 4-methylmorpholine (NMM, 43 mg, 0.427 mmol) at room temperature, causing the viscosity to increase temporarily. The reaction mixture was stirred at this temperature for 2 hours. To the solution was added 4-(4,6-dimethoxy-1,3,5-triazin-2-yl)-4-methylmorpholinium chloride (DMTMM, 169 mg, 0.61 mmol) was added and stirred for 72 hours at room temperature. NaCl (357 mg, 6.1 mmol) was then added to the reaction mixture, which was stirred for 1 hour and followed by the dropwise addition of acetone (400 mL) while stirring. The mixture was filtered. The filter cake was dissolved in 80 mL of deionized water and acetonitrile (V/V=3:1). NaCl (357 mg, 6.1 mmol) was then added to the reaction mixture, which was stirred for 1 hour and followed by the dropwise addition of acetone (200 mL) while stirring. The mixture was filtered. The filter cake was collected, washed with acetone, and dried in vacuo to give the title compound as a white solid. 0.29 g, Yield: 62.8%, DSR: 23%; ¹H NMR (400 MHz, D₂O) δ ppm 8.26-8.09 (m, 0.23H), 7.63-7.52 (m, 0.23H), 6.85-6.67 (m, 0.23H), 4.70-4.28 (m, 2.23H), 4.20-2.53 (m, 11.84H), 2.23-1.40 (mz, 4.61H), 1.34-1.06 (m, 0.46H).

With this step, reaction of Sodium hyaluronate (MW 2000 KDa) provided corresponding product (0.305 g, yield: 66.1%, DSR=31%).

### Example 64

### Preparation of conjugate between HA (Sodium hyaluronate) and methyl (2S)-6-amino-2-[[4-[1-[3-(cyanomethyl)-1-ethylsulfonyl-azetidin-3-yl]pyrazol-4-yl]pyrrolo[2,3-d]pyrimidine-7-carbonyl]amino]hexanoate

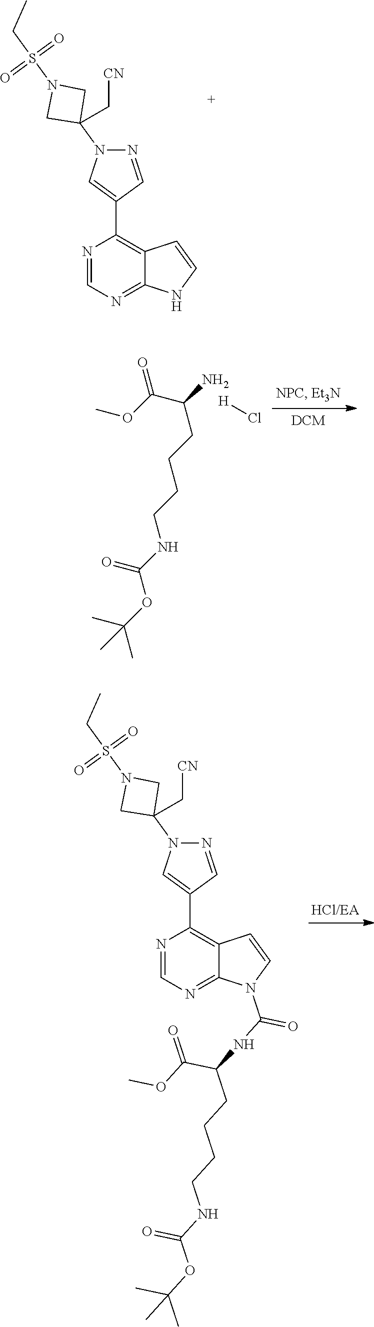

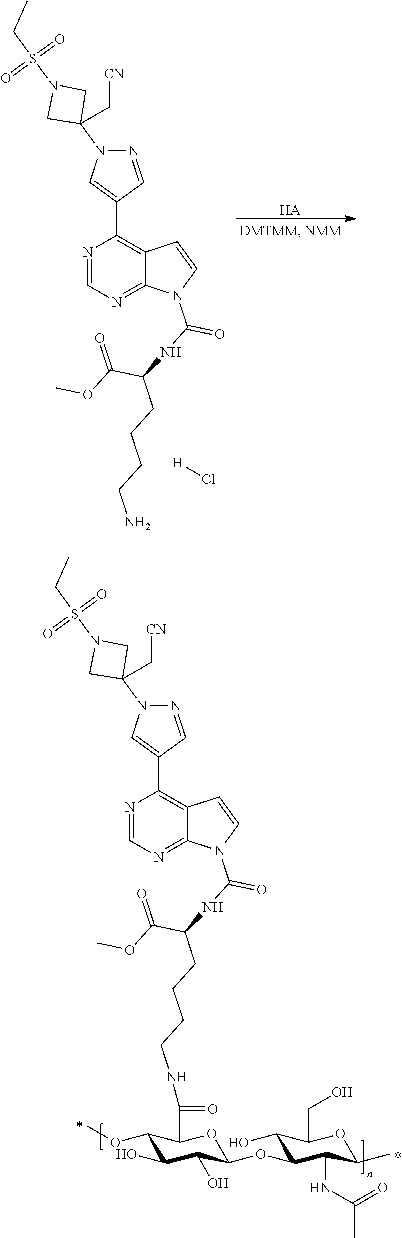

### Step 1: Preparation of methyl (2S)-6-(tert-butoxycarbonylamino)-2-[[4-[1-[3-(cyanomethyl)-1-ethylsulfonyl-azetidin-3-yl]pyrazol-4-yl]pyrrolo[2,3-d]pyrimidine-7-carbonyl]amino]Hexanoate

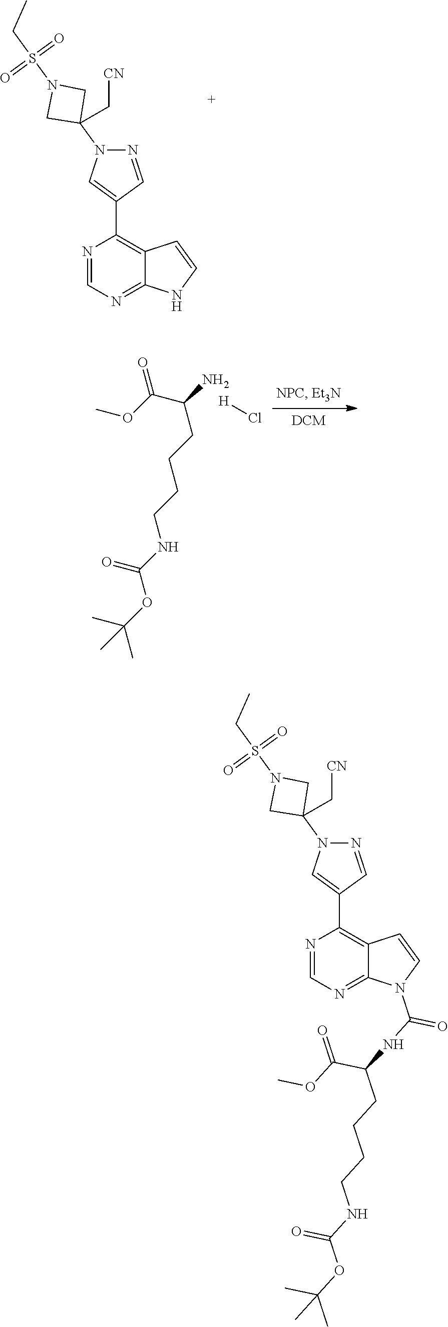

To a stirred mixture of 2-[1-ethylsulfonyl-3-[4-(7H-pyrrolo[2,3-d]pyrimidin-4-yl)pyrazol-1-yl]azetidin-3-yl]acetonitrile (1114 mg, 3 mmol) and bis(4-nitrophenyl) carbonate (1094 mg, 3.6 mmol) in DCM (40 mL) was added triethylamine (1.25 mL, 9 mmol). The reaction mixture was heated to 45° C. and stirred at this temperature for 5 hours. Then the reaction mixture was cooled to room temperature. (S)-Methyl 6-amino-2-((tert-butoxycarbonyl)-amino)hexanoate (1069 mg, 3.6 mmol) was added. The reaction mixture was stirred at 45° C. for 16 hours. The solution was diluted with DCM and washed with water. The organic layer was dried over sodium sulfate, filtered and then concentrated under reduced pressure. The crude residue was purified by column chromatography (DCM:Methanol=100:1) to afford the title compound (1.3 g, Yield: 65.9%). MS (m/z): [M+H]+ calcd for C₂₉H₃₉N₉O₇S, 658.26; found, 658.1. ¹H NMR (400 MHz, CDCl₃) δ ppm 9.93 (d, J=7.6 Hz, 1H), 8.95 (s, 1H), 8.50 (s, 1H), 8.35 (s, 1H), 8.04 (d, J=4.0 Hz, 1H), 6.85 (d, J=4.1 Hz, 1H), 5.45-5.34 (m, 1H), 4.76-4.71 (m, 1H), 4.66 (d, J=9.4 Hz, 2H), 4.63-4.56 (m, 1H), 4.52-4.42 (m, 1H), 4.28 (d, J=9.6 Hz, 2H), 3.84 (s, 3H), 3.44 (s, 2H), 3.20-3.07 (m, 2H), 2.10-1.94 (m, 2H), 1.88-1.76 (m, 1H), 1.61-1.35 (m, 12H), 1.27 (s, 3H).

### Step 2: Preparation of methyl (2S)-6-amino-2-[[4-[1-[3-(cyanomethyl)-1-ethylsulfonyl-azetidin-3-yl]pyrazol-4-yl]pyrrolo[2,3-d]pyrimidine-7-carbonyl]amino]hexanoate hydrochloride

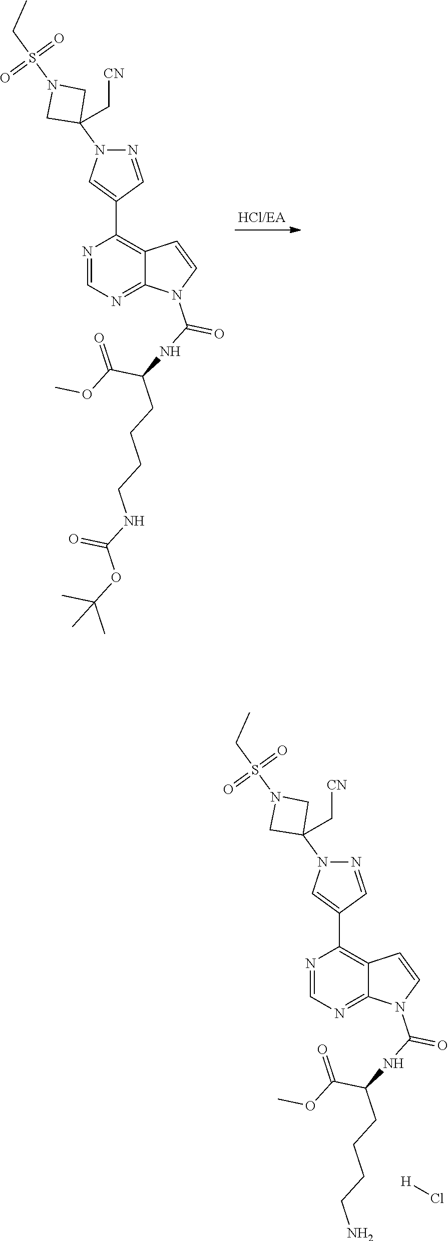

To a stirred solution of methyl (2S)-6-(tert-butoxycarbonylamino)-2-[[4-[1-[3-(cyanomethyl)-1-ethylsulfonyl-azetidin-3-yl]pyrazol-4-yl]pyrrolo[2,3-d]pyrimidine-7-carbonyl]amino]hexanoate (1.23 g, 1.87 mmol) in EtOAc (25 mL) was slowly added 4M HCl in ethyl acetate (commercially available) (4.92 mL) at ice-bath. The reaction mixture was allowed to cool to room temperature and then was stirred at room temperature for 16 hours. The solution was diluted with EtOAc and concentrated under reduced pressure to afford the title compound as a white solid (1.1 g, Yield: 99%) that was used without further purification. MS (m/z): [M+H]+ calcd for C₂₄H₃₁N₉O₅S, 558.21; found, 558.1.

### Step 3: Preparation of conjugate between HA (Sodium hyaluronate) and methyl (2S)-6-amino-2-[[4-[1-[3-(cyanomethyl)-1-ethylsulfonyl-azetidin-3-yl]pyrazol-4-yl]pyrrolo[2,3-d]pyrimidine-7-carbonyl]amino]hexanoate

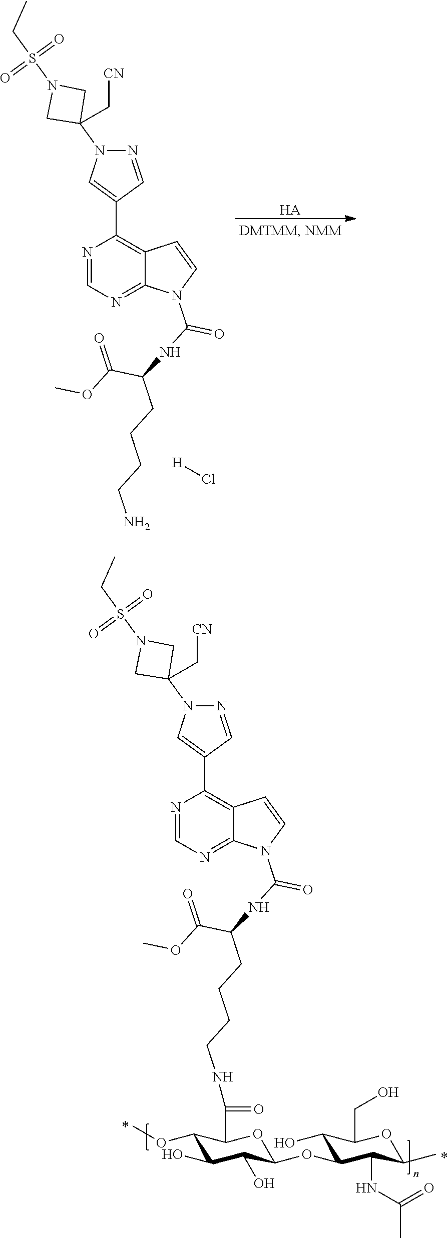

Sodium hyaluronate (175 mg, 0.434 mmol carboxylic acid, MW 50 kDa) was dissolved in 35 mL of deionized water in a 100 mL round-bottomed flask followed by the dropwise addition of 23 mL of acetonitrile while stirring. To the solution was added methyl (2S)-6-amino-2-[[4-[1-[3-(cyanomethyl)-1-ethylsulfonyl-azetidin-3-yl]pyrazol-4-yl]pyrrolo[2,3-d]pyrimidine-7-carbonyl]amino]hexanoate hydrochloride (200 mg, 0.317 mmol) and 4-methylmorpholine (NMM, 32 mg, 0.317 mmol) at room temperature, causing the viscosity to increase temporarily. The reaction mixture was stirred at this temperature for 2 hours. To the solution was added 4-(4,6-dimethoxy-1,3,5-triazin-2-yl)-4-methylmorpholinium chloride (DMTMM, 120 mg, 0.434 mmol) was added and stirred for 72 hours at room temperature. NaCl (254 mg, 4.34 mmol) was then added to the reaction mixture, which was stirred for 1 hour and followed by the dropwise addition of acetone (300 mL) while stirring. The mixture was filtered. The filter cake was dissolved in 80 mL of deionized water and acetonitrile (V/V=3:1). NaCl (254 mg, 4.34 mmol) was then added to the reaction mixture, which was stirred for 1 hour and followed by the dropwise addition of acetone (200 mL) while stirring. The mixture was filtered. The filter cake was collected, washed with acetone and dried in vacuo to give the title compound as a white solid. 0.2 g, Yield: 50.1%, DSR: 20%; ¹H NMR (400 MHz, D₂O) δ ppm 8.72-8.57 (m, 0.2H), 8.41-8.16 (m, 0.2H), 7.96-7.82 (m, 0.2H), 7.45-7.29 (m, 0.2H), 7.02-6.87 (m, 0.2H), 4.56-4.02 (m, 3.4H), 3.97-2.84 (m, 11.4H), 2.03-1.81 (m, 3.6H), 1.43 (m, 1.2H).

With this step, reaction of Sodium hyaluronate (MW 2000 KDa) provided corresponding product (0.154 g, yield: 38.6%, DSR=30%).

### Example 65

### Preparation of conjugate between HA (Sodium hyaluronate) and 4-[1-[3-(cyanomethyl)-1-ethylsulfonyl-azetidin-3-yl]pyrazol-4-yl]pyrrolo[2,3-d]pyrimidine-7-carbohydrazide

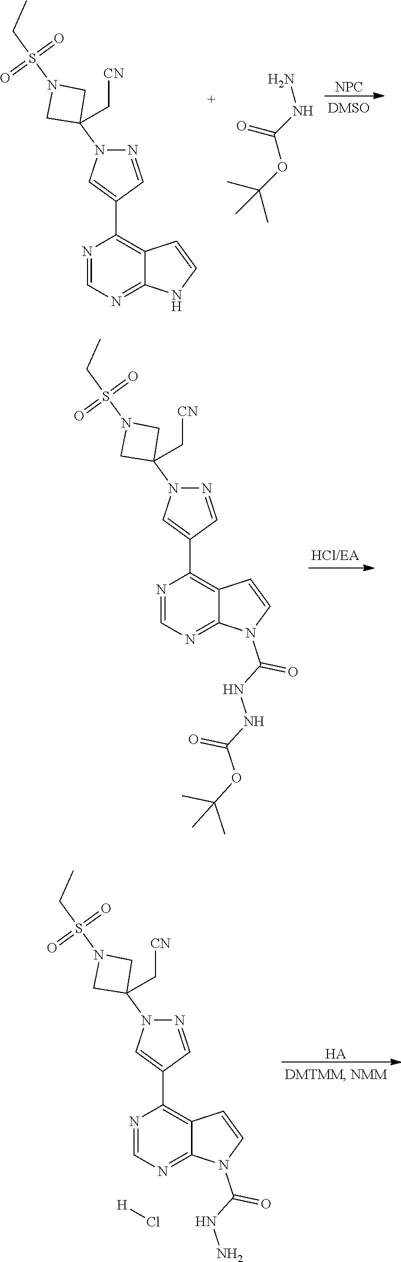

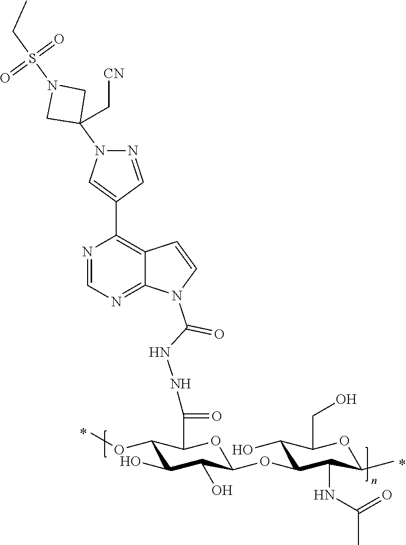

### Step 1: Preparation of tert-butyl N-[[4-[1-[3-(cyanomethyl)-1-ethylsulfonyl-azetidin-3-yl]pyrazol-4-yl]pyrrolo[2,3-d]pyrimidine-7-carbonyl]amino]carbamate

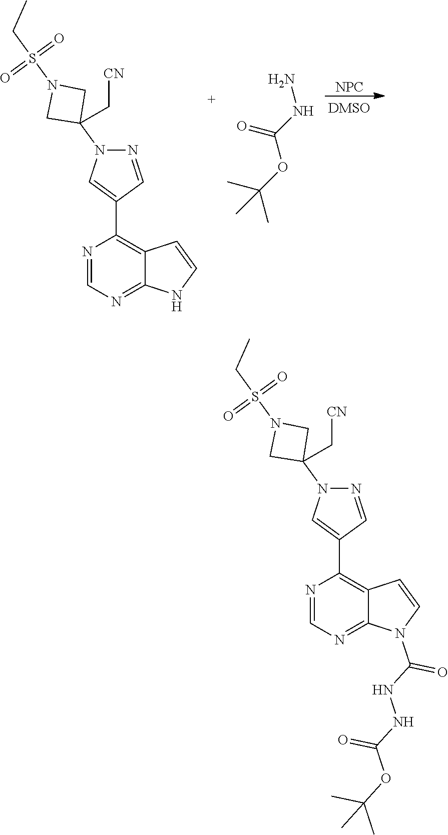

A mixture of 2-[1-ethylsulfonyl-3-[4-(7H-pyrrolo[2,3-d]pyrimidin-4-yl)pyrazol-1-yl]azetidin-3-yl]acetonitrile (1486 mg, 4 mmol) and bis(4-nitrophenyl) carbonate (1337.6 mg, 4.4 mmol) in DMSO (20 mL) was stirred at room temperature for 7 hours. Then tert-Butyl hydrazinecarboxylate (634.4 mg, 4.8 mmol) was added. The reaction mixture was stirred at room temperature for 16 hours. The reaction mixture was poured into saturated Na2CO3 solution. The mixture was filtered. The filter cake was washed with saturated NaHCO3 solution and water. Then the filter cake was dissolved in DCM and washed with water. The organic layer was dried over sodium sulfate, filtered and then concentrated under reduced pressure. The crude residue was purified by column chromatography (DCM:Methanol=100:1) to afford the title compound (0.758 g, Yield: 35.8%). MS (m/z): [M+H]+ calcd for C₂₂H₂₇N₉O₅S, 530.18; found, 530.1. ¹H NMR (400 MHz, CDCl₃) δ ppm 10.93 (d, J=1.8 Hz, 1H), 8.87 (s, 1H), 8.48 (s, 1H), 8.32 (s, 1H), 7.95 (dd, J=28.4, 4.1 Hz, 1H), 6.82 (t, J=17.0 Hz, 1H), 6.70 (s, 1H), 4.64 (d, J=9.3 Hz, 2H), 4.23 (t, J=23.2 Hz, 2H), 3.42 (s, 2H), 3.09 (q, J=7.4 Hz, 2H), 1.52 (s, 9H), 1.42 (t, J=7.4 Hz, 3H).

### Step 2: Preparation of 4-[1-[3-(cyanomethyl)-1-ethylsulfonyl-azetidin-3-yl]pyrazol-4-yl]pyrrolo[2,3-d]pyrimidine-7-carbohydrazide trifluoroacetate

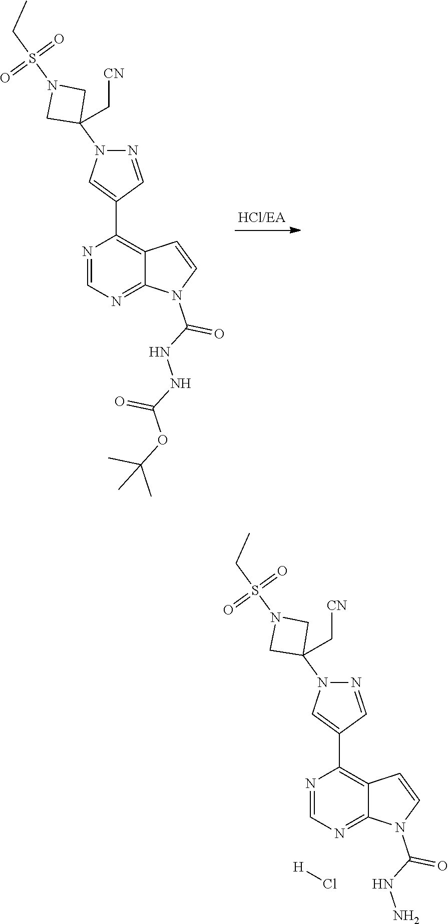 [SMILES: CCS(=O)(=O)N1CC(CC#N)(n2cc(-c3ncnc4c3ccn4C(=O)NNC(=O)OC(C)(C)C)cn2)C1 ; CCS(=O)(=O)N1CC(CC#N)(n2cc(-c3ncnc4c3ccn4C(=O)NN)cn2)C1 ; Cl]

To a stirred mixture of tert-butyl N-[[4-[1-[3-(cyanomethyl)-1-ethylsulfonyl-azetidin-3-yl]pyrazol-4-yl]pyrrolo[2,3-d]pyrimidine-7-carbonyl]amino]carbamate (0.6 g, 1.13 mmol) in DCM (9.6 mL) was slowly added trifluoroacetic acid (2.4 mL) at ice-bath. The reaction mixture was allowed to cool to room temperature and then was stirred at room temperature for 16 hours. The solution was concentrated under reduced pressure to afford the title compound as a white solid (0.61 g, Yield: 99%) that was used without further purification. MS (m/z): [M+H]+ calcd for C₁₇H₁₉N₉O₃S, 430.13; found, 430.0.

### Step 3: Preparation of conjugate between HA (Sodium hyaluronate) and 4-[1-[3-(cyanomethyl)-1-ethylsulfonyl-azetidin-3-yl]pyrazol-4-yl]pyrrolo[2,3-d]pyrimidine-7-carbohydrazide

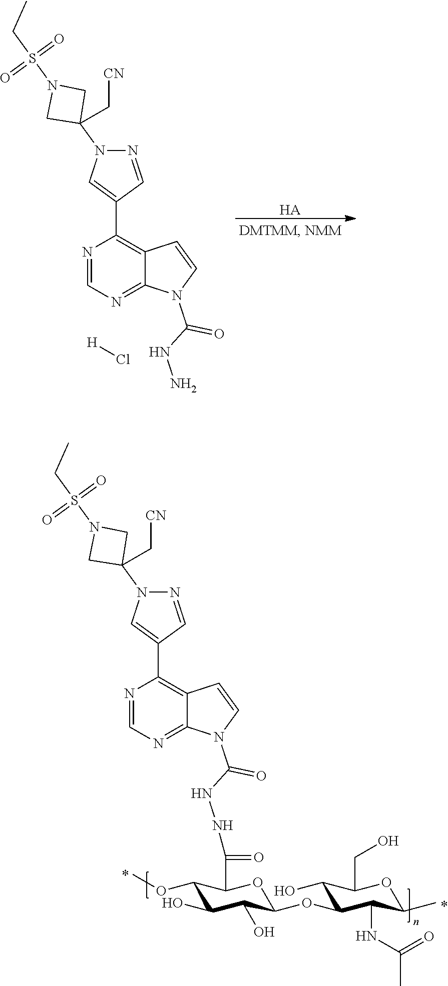

Sodium hyaluronate (212 mg, 0.526 mmol carboxylic acid, MW 50 kDa) was dissolved in 42.4 mL of deionized water in a 100 mL round-bottomed flask followed by the dropwise addition of 28 mL of acetonitrile while stirring. To the solution was added 4-[1-[3-(cyanomethyl)-1-ethylsulfonyl-azetidin-3-yl]pyrazol-4-yl]pyrrolo[2,3-d]pyrimidine-7-carbohydrazide hydrochloride (200 mg, 0.368 mmol) and 4-methylmorpholine (NMM, 37 mg, 0.368 mmol) at room temperature, causing the viscosity to increase temporarily. The reaction mixture was stirred at this temperature for 2 hours. To the solution was added 4-(4,6-dimethoxy-1,3,5-triazin-2-yl)-4-methylmorpholinium chloride (DMTMM, 146 mg, 0.526 mmol) was added and stirred for 72 hours at room temperature. NaCl (308 mg, 5.26 mmol) was then added to the reaction mixture, which was stirred for 1 hour and followed by the dropwise addition of acetone (300 mL) while stirring. The mixture was filtered. The filter cake was dissolved in 80 mL of deionized water and acetonitrile (V/V=3:1). NaCl (308 mg, 5.26 mmol) was then added to the reaction mixture, which was stirred for 1 hour and followed by the dropwise addition of acetone (200 mL) while stirring. The mixture was filtered. The filter cake was collected, washed with acetone and dried in vacuo to give the title compound as a white solid. 0.2 g, Yield: 48.1%, DSR: 40%; ¹H NMR (400 MHz, D₂O) δ ppm 8.89-8.663 (m, 0.4H), 8.49-8.17 (m, 0.4H), 8.04-7.84 (m, 0.4H), 7.05-6.91 (m, 0.4H), 4.43 (d, J=34.6 Hz, 3.6H), 3.46 (dd, J=86.0, 63.7 Hz, 11.6H), 1.93 (s, 3H), 1.26 (d, J=43.7 Hz, 1.2H).

With this step, reaction of Sodium hyaluronate (MW 2000 KDa) provided corresponding product (0.154 g, yield: 37%, DSR=40%).

### Example 66

### Preparation of conjugate between HA (Sodium hyaluronate) and methyl (2S)-6-amino-2-[[4-[1-[(1R)-2-cyano-1-cyclopentyl-ethyl]pyrazol-4-yl]pyrrolo[2,3-d]pyrimidine-7-carbonyl]amino]hexanoate

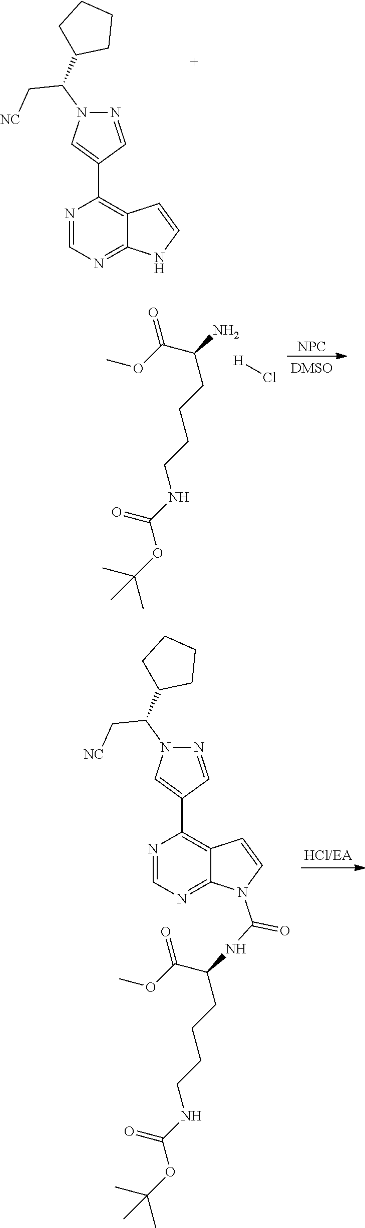

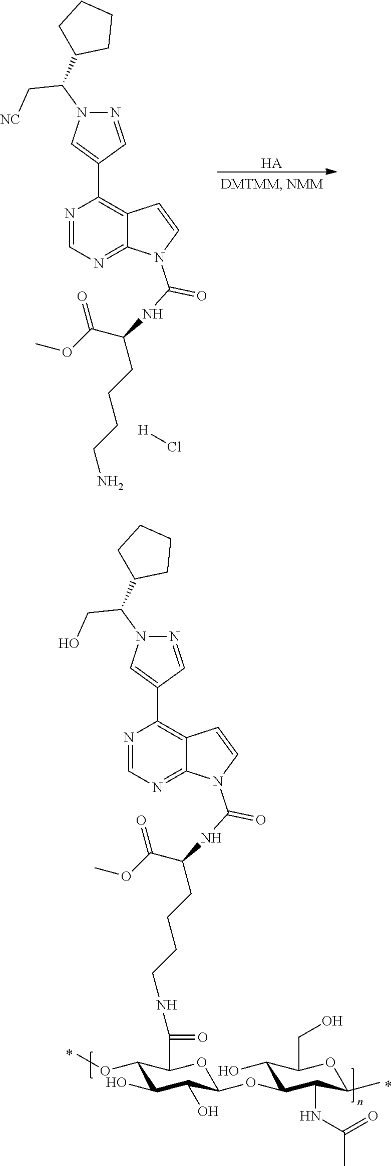

### Step 1: Preparation of methyl (2S)-6-(tert-butoxycarbonylamino)-2-[[4-[1-[(1R)-2-cyano-1-cyclopentyl-ethyl]pyrazol-4-yl]pyrrolo[2,3-d]pyrimidine-7-carbonyl]amino]hexanoate

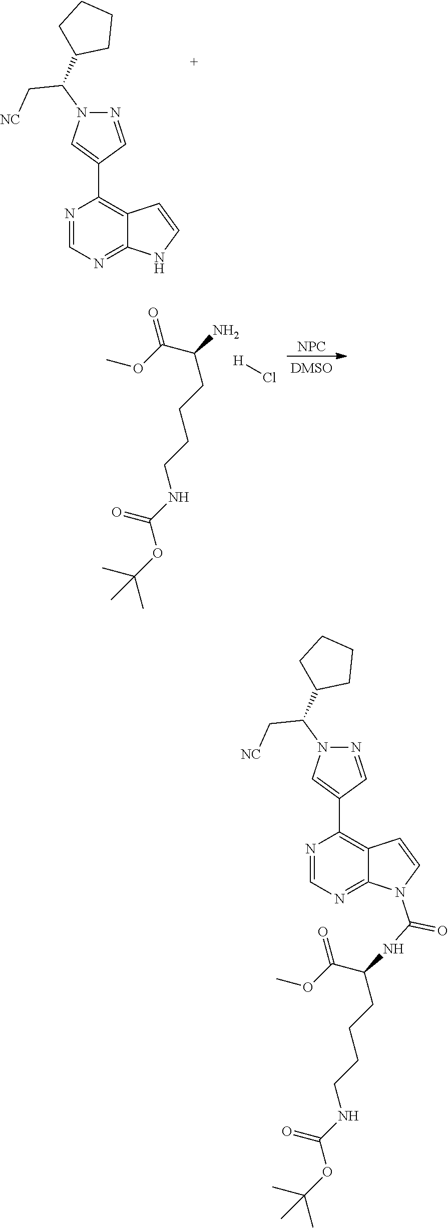

A mixture of (3R)-3-cyclopentyl-3-[4-(7H-pyrrolo[2,3-d]pyrimidin-4-yl)pyrazol-1-yl]propanenitrile (1486 mg, 4 mmol) and bis(4-nitrophenyl) carbonate mg, 3.754 mmol) in DMSO (12 mL) was stirred at room temperature for 7 hours. Then (S)-methyl 6-amino-2-((tert-butoxycarbonyl)-amino)hexanoate (1448 mg, 4.88 mmol) was added. The reaction mixture was stirred at room temperature for 16 hours. The reaction mixture was poured into saturated Na₂CO₃ solution. The mixture was filtered. The filter cake was washed with saturated NaHCO₃ solution and water. Then the filter cake was dissolved in DCM and washed with water. The organic layer was dried over sodium sulfate, filtered and then concentrated under reduced pressure. The crude residue was purified by column chromatography (DCM:Methanol=150:1) to afford the title compound (0.828 g, Yield: 34.9%). MS (m/z): [M+H]+ calcd for C₃₀H₄₀N₈O₅, 593.31; found, 593.2. ¹H NMR (400 MHz, CDCl3) δ ppm 9.95 (d, J=7.5 Hz, 1H), 8.91 (s, 1H), 8.32 (d, J=11.9 Hz, 2H), 7.99 (d, J=4.0 Hz, 1H), 6.82 (d, J=4.0 Hz, 1H), 4.76 (dd, J=12.8, 7.5 Hz, 1H), 4.60 (s, 1H), 4.28 (td, J=9.9, 3.8 Hz, 1H), 3.81 (s, 3H), 3.22-3.05 (m, 3H), 2.97 (dd, J=17.0, 3.8 Hz, 1H), 2.61 (dt, J=16.5, 8.4 Hz, 1H), 2.13-1.88 (m, 3H), 1.65-1.50 (m, 7H), 1.44-1.35 (m, 9H), 1.27 (d, J=12.8 Hz, 4H).

### Step 2: Preparation of methyl (2S)-6-amino-2-[[4-[1-[(1R)-2-cyano-1-cyclopentyl-ethyl]pyrazol-4-yl]pyrrolo[2,3-d]pyrimidine-7-carbonyl]amino]hexanoate hydrochloride

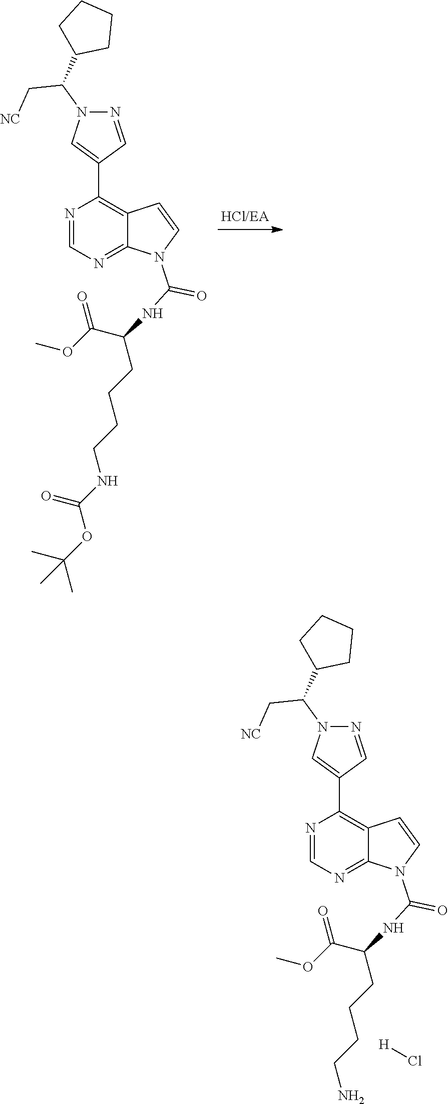

To a stirred solution of methyl (2S)-6-(tert-butoxycarbonylamino)-2-[[4-[1-[(1R)-2-cyano-1-cyclopentyl-ethyl]pyrazol-4-yl]pyrrolo[2,3-d]pyrimidine-7-carbonyl]amino]hexanoate (0.6 g, 1.01 mmol) in EtOAc (12 mL) was slowly added 4M HCl in ethyl acetate (commercially available) (2.4 mL) at ice-bath. The reaction mixture was allowed to cool to room temperature and then was stirred at room temperature for 16 hours. The solution was diluted with EtOAc and concentrated under reduced pressure to afford the title compound as a white solid (0.53 g, Yield: 99%) that was used without further purification. MS (m/z): [M+H]+ calcd for C₂₅H₃₂N₈O₃, 493.25; found, 493.2.

### Step 3: Preparation of conjugate between HA (Sodium hyaluronate) and methyl (2S)-6-amino-2-[[4-[1-[(1R)-2-cyano-1-cyclopentyl-ethyl]pyrazol-4-yl]pyrrolo[2,3-d]pyrimidine-7-carbonyl]amino]hexanoate

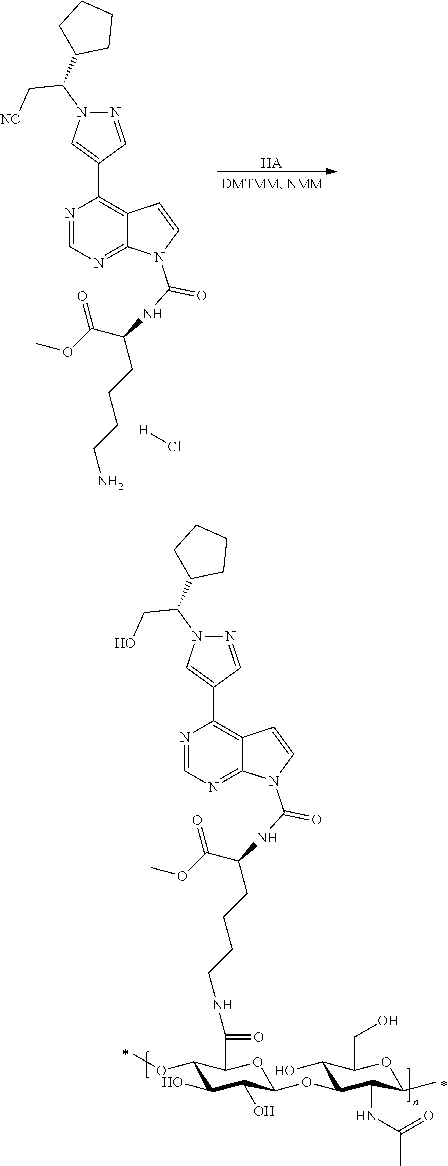

Sodium hyaluronate (204 mg, 0.506 mmol carboxylic acid, MW 50 kDa) was dissolved in 40.8 mL of deionized water in a 100 mL round-bottomed flask followed by the dropwise addition of 26.5 mL of acetonitrile while stirring. To the solution was added methyl (2S)-6-amino-2-[[4-[1-[(1R)-2-cyano-1-cyclopentyl-ethyl]pyrazol-4-yl]pyrrolo[2,3-d]pyrimidine-7-carbonyl]amino]hexanoate hydrochloride (200 mg, 0.354 mmol) and 4-methylmorpholine (NMM, 36 mg, 0.354 mmol) at room temperature, causing the viscosity to increase temporarily. The reaction mixture was stirred at this temperature for 2 hours. To the solution was added 4-(4,6-dimethoxy-1,3,5-triazin-2-yl)-4-methylmorpholinium chloride (DMTMM, 140 mg, 0.506 mmol) was added and stirred for 72 hours at room temperature. NaCl (207 mg, 3.54 mmol) was then added to the reaction mixture, which was stirred for 1 hour and followed by the dropwise addition of acetone (300 mL) while stirring. The mixture was filtered. The filter cake was dissolved in 80 mL of deionized water and acetonitrile (V/V=3:1).NaCl (207 mg, 3.54 mmol) was then added to the reaction mixture, which was stirred for 1 hour and followed by the dropwise addition of acetone (200 mL) while stirring. The mixture was filtered. The filter cake was collected, washed with acetone and dried in vacuo to give the title compound as a white solid. 0.195 g, Yield: 45.1%, DSR: 27%; ¹H NMR (400 MHz, D₂O) δ ppm 8.59-8.45 (m, 0.27H), 8.33-7.88 (m, 0.81H), 6.91-6.80 (m, 0.27H), 4.57-4.02 (m, 2.54H), 3.97-2.72 (m, 11.89H), 2.05-1.72 (m, 3.81H), 1.70-0.99 (m, 2.97H).

With this step, reaction of Sodium hyaluronate (MW 2000 KDa) provided corresponding product (0.197 g, yield: 45.5%, DSR=16%).

### Example 67

### Preparation of conjugate between HA (Sodium hyaluronate) and 4-[1-[(1R)-2-cyano-1-cyclopentyl-ethyl]pyrazol-4-yl]pyrrolo[2,3-d]pyrimidine-7-carbohydrazide

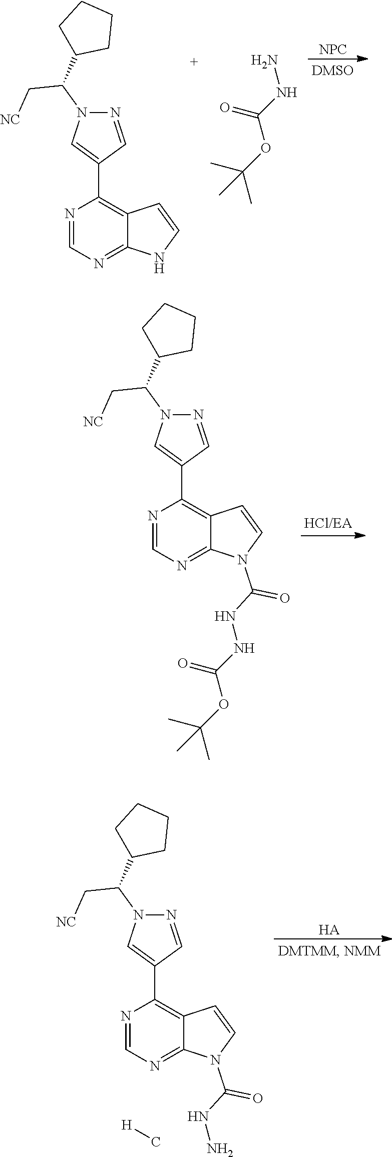

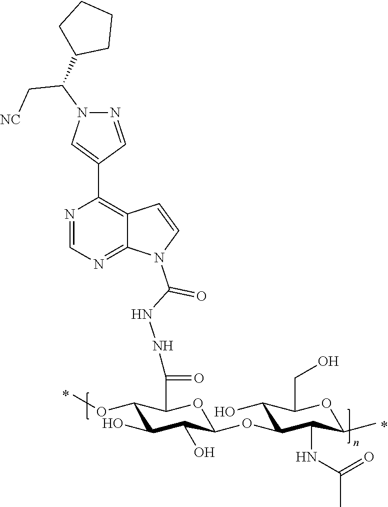

### Step 1: Preparation of tert-butyl N-[[4-[1-[(1R)-2-cyano-1-cyclopentyl-ethyl]pyrazol-4-yl]pyrrolo[2,3-d]pyrimidine-7-carbonyl]amino]carbamate

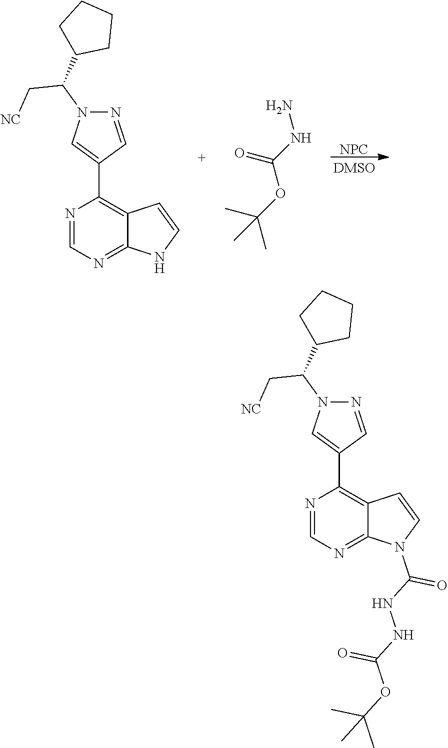

A mixture of 2-[1-ethylsulfonyl-3-[4-(7H-pyrrolo[2,3-d]pyrimidin-4-yl)pyrazol-1-yl]azetidin-3-yl]acetonitrile (1617 mg, 4 mmol), triethylamine (1.67 mL, 12 mmol) and bis(4-nitrophenyl) carbonate (1337.6 mg, 4.4 mmol) in DMSO (20 mL) was stirred at room temperature for 7 hours. Then tert-Butyl hydrazinecarboxylate (634.4 mg, 4.8 mmol) was added. The reaction mixture was stirred at room temperature for 16 hours. The reaction mixture was poured into saturated Na2CO3 solution. The mixture was filtered. The filter cake was washed with saturated NaHCO₃ solution and water. Then the filter cake was dissolved in DCM and washed with water. The organic layer was dried over sodium sulfate, filtered and then concentrated under reduced pressure. The crude residue was purified by column chromatography (DCM:Methanol=100:1) to afford the title compound (1.02 g, Yield: 54.9%). MS (m/z): [M+H]+ calcd for C₂₃H₂₈N₈O₃, 465.22; found, 465.2. ¹H NMR (400 MHz, CDCl3) δ ppm 10.97 (d, J=1.9 Hz, 1H), 8.86 (s, 1H), 8.31 (d, J=15.2 Hz, 2H), 7.97 (d, J=4.0 Hz, 1H), 6.87-6.84 (m, 1H), 6.62 (s, 1H), 4.27 (td, J=10.0, 3.9 Hz, 1H), 3.14 (dd, J=17.0, 8.6 Hz, 1H), 2.97 (dd, J=17.0, 3.9 Hz, 1H), 2.61 (dt, J=17.0, 8.6 Hz, 1H), 2.04-1.92 (m, 1H), 1.79-1.62 (m, 5H), 1.54 (d, J=13.3 Hz, 9H), 1.35-1.19 (m, 3H).

### Step 2: Preparation of 4-[1-[(1R)-2-cyano-1-cyclopentyl-ethyl]pyrazol-4-yl]pyrrolo[2,3-d]pyrimidine-7-carbohydrazide hydrochloride

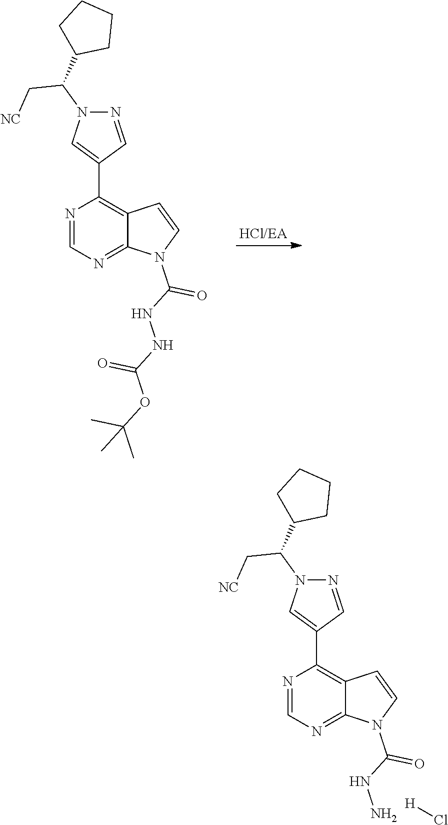

To a stirred solution of tert-butyl N-[[4-[1-[(1R)-2-cyano-1-cyclopentyl-ethyl]pyrazol-4-yl]pyrrolo[2,3-d]pyrimidine-7-carbonyl]amino]carbamate (0.75 g, 1.61 mmol) in EtOAc (15 mL) was slowly added 4M HCl in ethyl acetate (commercially available) (3 mL) at ice-bath. The reaction mixture was allowed to cool to room temperature and then was stirred at room temperature for 16 hours. The solution was diluted with EtOAc and concentrated under reduced pressure to afford the title compound as a white solid (0.557 g, Yield: 95%) that was used without further purification. MS (m/z): [M+H]⁺ calcd for C₁₈H₂₀N₈O, 365.17; found, 365.1.

### Step 3: Preparation of conjugate between HA (Sodium hyaluronate) and 4-[1-[(1R)-2-cyano-1-cyclopentyl-ethyl]pyrazol-4-yl]pyrrolo[2,3-d]pyrimidine-7-carbohydrazide

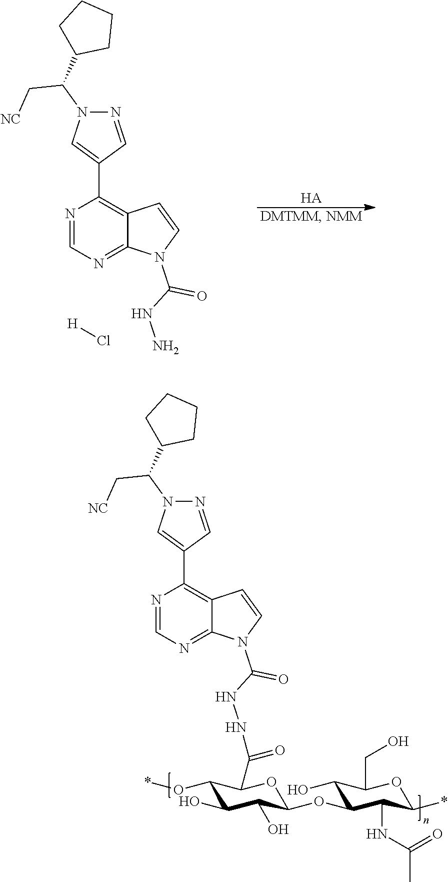

Sodium hyaluronate (263.5 mg, 0.654 mmol carboxylic acid, MW 50 KDa) was dissolved in 53 mL of deionized water in a 100 mL round-bottomed flask followed by the dropwise addition of 34 mL of acetonitrile while stirring. To the solution was added 4-[1-[(1R)-2-cyano-1-cyclopentyl-ethyl]pyrazol-4-yl]pyrrolo[2,3-d]pyrimidine-7-carbohydrazide hydrochloride (200 mg, 0.458 mmol) and 4-methylmorpholine (NMM, 46 mg, 0.458 mmol) at room temperature, causing the viscosity to increase temporarily. The reaction mixture was stirred at this temperature for 2 hours. To the solution was added 4-(4,6-dimethoxy-1,3,5-triazin-2-yl)-4-methylmorpholinium chloride (DMTMM, 181 mg, 0.654 mmol) was added and stirred for 72 hours at room temperature. NaCl (382 mg, 6.54 mmol) was then added to the reaction mixture, which was stirred for 1 hour and followed by the dropwise addition of acetone (300 mL) while stirring. The mixture was filtered. The filter cake was dissolved in 80 mL of deionized water and acetonitrile (V/V=3:1). NaCl (382 mg, 6.54 mmol) was then added to the reaction mixture, which was stirred for 1 hour and followed by the dropwise addition of acetone (200 mL) while stirring. The mixture was filtered. The filter cake was collected, washed with acetone, and dried in vacuo to give the title compound as a white solid. 0.316 g, Yield: 66.5%, DSR: 33%; ¹H NMR (400 MHz, D₂O) δ ppm 8.88-8.65 (m, 0.33H), 8.45-7.28 (m, 0.99H), 7.07-6.82 (m, 0.33H), 4.68-4.27 (m, 2.33H), 4.21-2.62 (m, 10.66H), 2.47-2.23 (m, 0.33H), 2.15-1.32 (m, 4.32H), 1.31-1.02 (m, 0.99H).

With this step, reaction of Sodium hyaluronate (MW 2000 KDa) provided corresponding product (0.308 g, yield: 64.8%, DSR=21%).

### Example 68

### Preparation of conjugate between HA (Sodium hyaluronate) and [4-[[(3R,4R)-1-(2-cyanoacetyl)-4-methyl-3-piperidyl]-methyl-amino]pyrrolo[2,3-d]pyrimidin-7-yl]methyl 2-aminoacetate

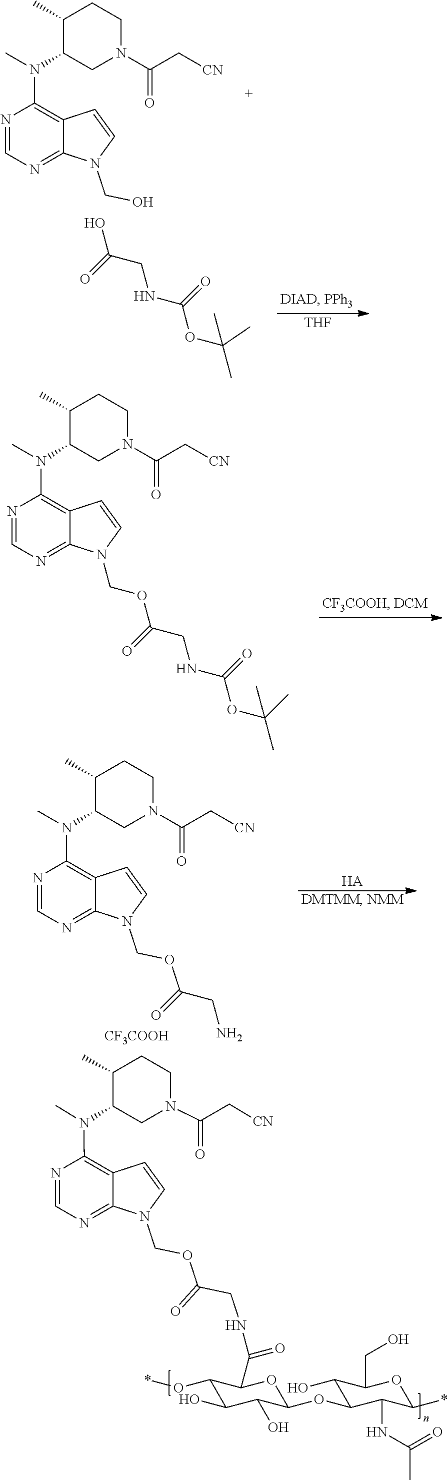

### Step 1: Preparation of [4-[[(3R,4R)-1-(2-cyanoacetyl)-4-methyl-3-piperidyl]-methyl-amino]pyrrolo[2,3-d]pyrimidin-7-yl]methyl 2-(tert-butoxycarbonylamino)acetate

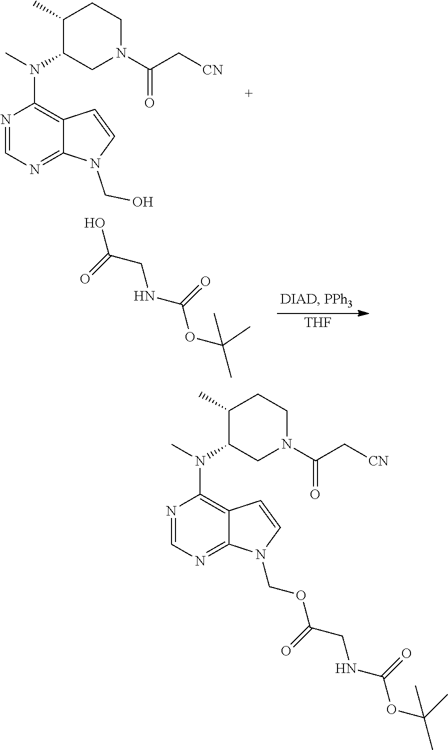

A mixture of 3-[(3R,4R)-3-[[7-(hydroxymethyl)pyrrolo[2,3-d]pyrimidin-4-yl]-methyl-amino]-4-methyl-1-piperidyl]-3-oxo-propanenitrile (600 mg, 1.754 mmol), 2-(tert-butoxycarbonylamino)acetic acid (1.23 mg, 7.02 mmol) and triphenylphosphine (1840 mg, 7.02 mmol) in THE (120 mL) was stirred at 0° C.-10° C. for 10 mins. Then diisopropyl azodicarboxylate (1420 mg, 7.02 mmol) was added dropwise. The reaction mixture was stirred at this temperature for 16 hours. The reaction mixture was poured into saturated NaHCO₃ solution at ice-bath. The mixture was diluted with EtOAc and washed with saturated brine solution. The organic layer was dried over sodium sulfate, filtered and then concentrated under reduced pressure. The crude residue was purified by column chromatography (EtOAc:Hexane=1:2) to afford the title compound (400 mg, Yield: 45.7%). MS (m/z): [M+H]+ calcd for C₂₄H₃₃N₇O₅, 500.25; found, 500.1. ¹H NMR (400 MHz, CDCl3) δ ppm 8.31 (t, J=16.1 Hz, 1H), 7.15 (dd, J=13.4, 3.7 Hz, 1H), 6.56 (t, J=3.9 Hz, 1H), 6.23 (d, J=2.8 Hz, 2H), 5.14 (s, 1H), 4.96 (s, 1H), 4.10-3.98 (m, 1H), 3.97-3.76 (m, 3H), 3.68-3.45 (m, 4H), 3.42-3.30 (m, 3H), 2.57-2.43 (m, 1H), 2.01-1.85 (m, 1H), 1.82-1.69 (m, 1H), 1.51-1.32 (m, 9H), 1.27-1.24 (m, 2H), 1.13-1.09 (m, 1H).

### Step 2: Preparation of [4-[[(3R,4R)-1-(2-cyanoacetyl)-4-methyl-3-piperidyl]-methyl-amino]pyrrolo[2,3-d]pyrimidin-7-yl]methyl 2-aminoacetate trifluoroacetate

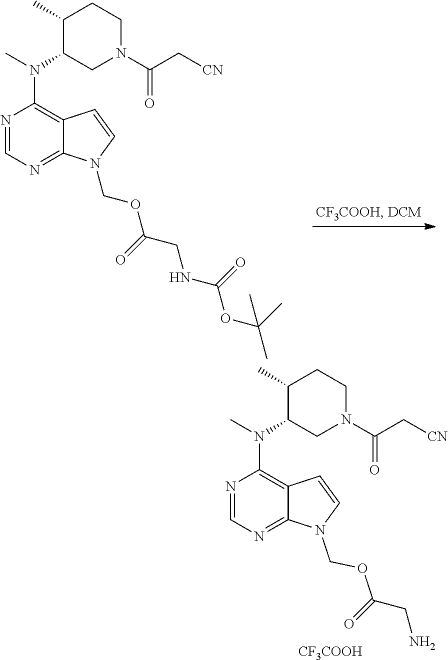

To a stirred mixture of [4-[[(3R,4R)-1-(2-cyanoacetyl)-4-methyl-3-piperidyl]-methyl-amino]pyrrolo[2,3-d]pyrimidin-7-yl]methyl 2-(tert-butoxycarbonylamino)acetate (0.35 g, 0.7 mmol) in DCM (10 mL) was slowly added trifluoroacetic acid (2 mL) at ice-bath. The reaction mixture was allowed to cool to room temperature and then was stirred at room temperature for 16 hours. The solution was concentrated under reduced pressure to afford the title compound as a white solid (0.27 g, Yield: 96.7%) that was used without further purification. MS (m/z): [M+H]+ calcd for C₁₉H₂₅N₇O₃, 400.20; found, 400.1.

### Step 3: Preparation of conjugate between HA (Sodium hyaluronate) and [4-[[(3R,4R)-1-(2-cyanoacetyl)-4-methyl-3-piperidyl]-methyl-amino]pyrrolo[2,3-d]pyrimidin-7-yl]methyl 2-aminoacetate

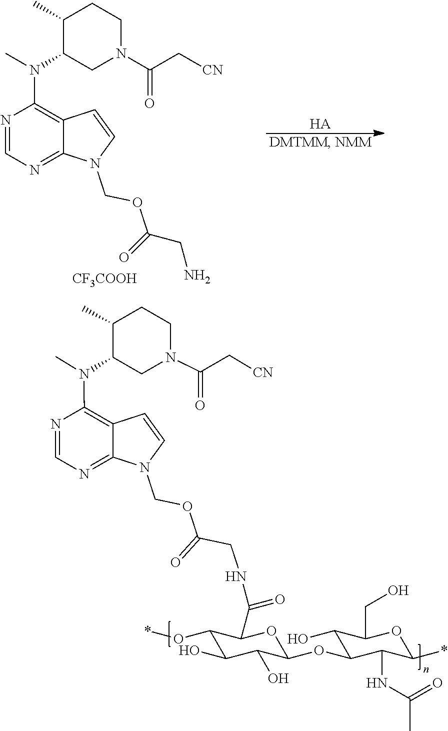

Sodium hyaluronate (178 mg, 0.443 mmol carboxylic acid, MW 50 kDa) was dissolved in 53 mL of deionized water in a 100 mL round-bottomed flask. To the solution was added [4-[[(3R,4R)-1-(2-cyanoacetyl)-4-methyl-3-piperidyl]-methyl-amino]pyrrolo[2,3-d]pyrimidin-7-yl]methyl 2-aminoacetate trifluoroacetate (150 mg, 0.443 mmol) and 4-methylmorpholine (NMM, 44.8 mg, 0.443 mmol) at room temperature, causing the viscosity to increase temporarily. The reaction mixture was stirred at this temperature for 2 hours. To the solution was added 4-(4,6-dimethoxy-1,3,5-triazin-2-yl)-4-methylmorpholinium chloride (DMTMM, 122 mg, 0.443 mmol) was added and stirred for 72 hours at room temperature. NaCl (259 mg, 4.43 mmol) was then added to the reaction mixture, which was stirred for 1 hour and followed by the dropwise addition of acetone (300 mL) while stirring. The mixture was filtered. The filter cake was dissolved in 80 mL of deionized water and acetonitrile (V/V=3:1). NaCl (259 mg, 4.43 mmol) was then added to the reaction mixture, which was stirred for 1 hour and followed by the dropwise addition of acetone (200 mL) while stirring. The mixture was filtered. The filter cake was collected, washed with acetone and dried in vacuo to give the title compound as a white solid. 0.15 g, Yield: 44.5%, DSR: 21%; ¹H NMR (400 MHz, D₂O) δ ppm 8.24-8.11 (m, 0.21H), 7.40-7.27 (m, 0.21H), 6.78-6.67 (m, 0.21H), 6.31-6.07 (m, 0.42H), 4.59-4.17 (m, 2.21H), 4.11-2.95 (m, 12.31H), 2.49-2.35 (m, 0.21H), 2.00-1.61 (m, 3.42H), 1.27-1.16 (m, 0.21H), 1.07-0.90 (m, 0.42H).

With this step, reaction of Sodium hyaluronate (MW 2000 KDa) provided corresponding product (0.108 g, yield: 45.7%, DSR=19%).

### Example 69

### Preparation of conjugate between HA (Sodium hyaluronate) and [4-[[(3R,4R)-1-(2-cyanoacetyl)-4-methyl-3-piperidyl]-methyl-amino]pyrrolo[2,3-d]pyrimidin-7-yl]methyl 4-aminobutanoate

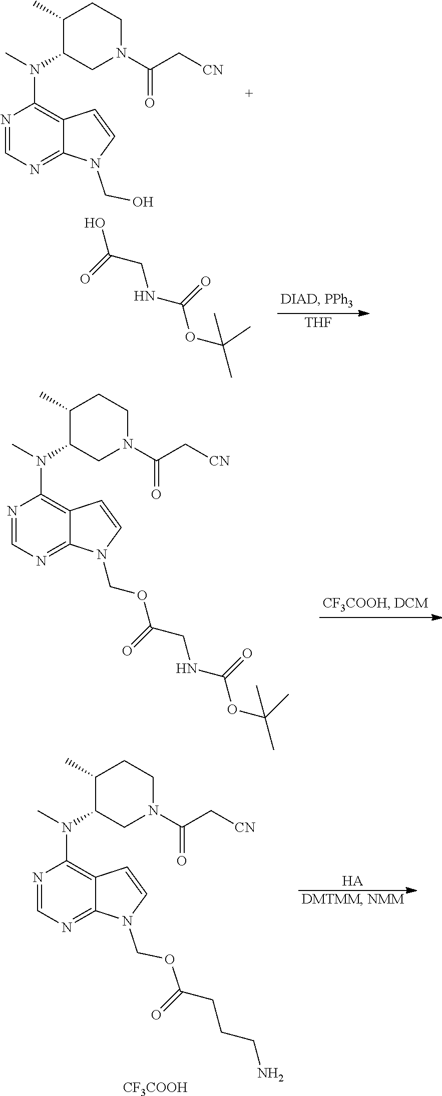

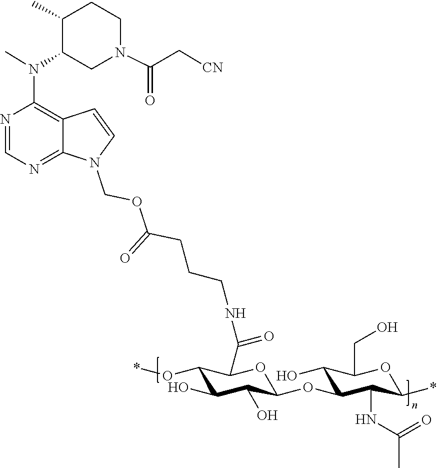

### Step 1: Preparation of [5-[[(3R,4R)-1-(2-cyanoacetyl)-4-methyl-3-piperidyl]-methyl-amino]pyrrolo[2,3-d]pyrimidin-7-yl]methyl 4-(tert-butoxycarbonylamino)butanoate

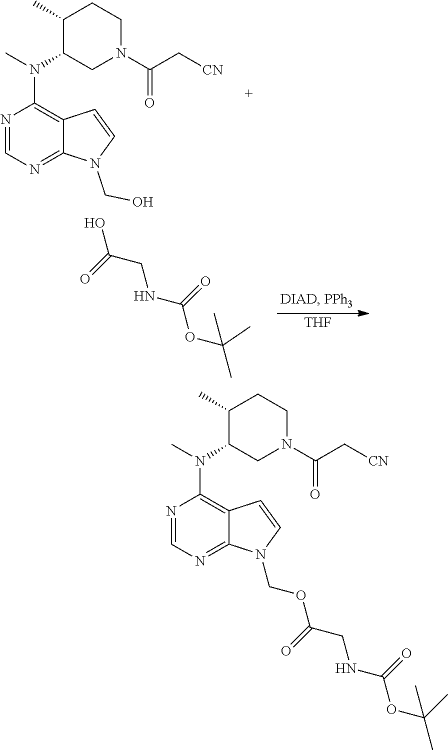

A mixture of 3-[(3R,4R)-3-[[7-(hydroxymethyl)pyrrolo[2,3-d]pyrimidin-4-yl]-methyl-amino]-4-methyl-1-piperidyl]-3-oxo-propanenitrile (300 mg, 0.877 mmol), 4-(tert-butoxycarbonylamino)butanoic acid (713 mg, 3.51 mmol) and triphenylphosphine (920 mg, 3.51 mmol) in THE (60 mL) was stirred at 0° C.-10° C. for 10 mins. Then diisopropyl azodicarboxylate (709.5 mg, 3.51 mmol) was added dropwise. The reaction mixture was stirred at this temperature for 16 hours. The reaction mixture was poured into saturated NaHCO₃ solution at ice-bath. The mixture was diluted with EtOAc and washed with saturated brine solution. The organic layer was dried over sodium sulfate, filtered and then concentrated under reduced pressure. The crude residue was purified by column chromatography (EtOAc:Hexane=1:2) to afford the title compound (240 mg, Yield: 51.9%). MS (m/z): [M+H]+ calcd for C₂₆H₃₇N₇O₅, 528.28; found, 528.1. ¹H NMR (400 MHz, CDCl3) δ ppm 8.34 (d, J=7.5 Hz, 1H), 7.16 (dd, J=13.8, 3.7 Hz, 1H), 6.55 (t, J=4.1 Hz, 1H), 6.17 (d, J=3.4 Hz, 2H), 5.14 (s, 1H), 4.61 (s, 1H), 4.09-3.99 (m, 1H), 3.89-3.56 (m, 3H), 3.54-3.31 (m, 5H), 3.12 (d, J=6.1 Hz, 2H), 2.50 (d, J=26.3 Hz, 1H), 2.37 (t, J=7.3 Hz, 2H), 2.01-1.86 (m, 1H), 1.78 (dd, J=14.2, 7.1 Hz, 2H), 1.50-1.30 (m, 9H), 1.31-1.21 (m, 3H), 1.13-1.09 (m, 1H).

### Step 2: Preparation of [4-[[(3R,4R)-1-(2-cyanoacetyl)-4-methyl-3-piperidyl]-methyl-amino]pyrrolo[2,3-d]pyrimidin-7-yl]methyl 4-aminobutanoate trifluoroacetate

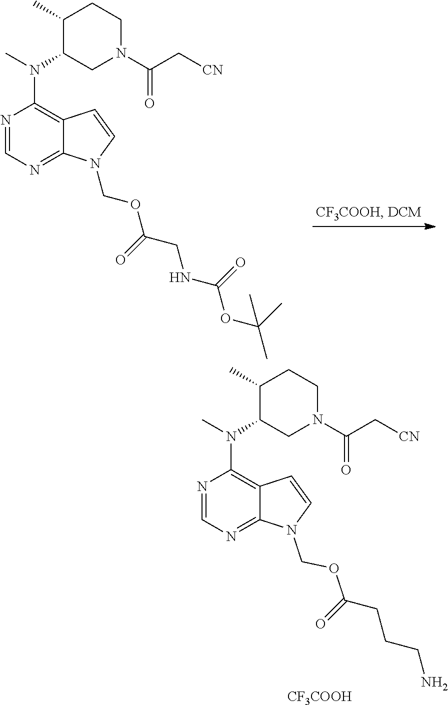

To a stirred mixture of [5-[[(3R,4R)-1-(2-cyanoacetyl)-4-methyl-3-piperidyl]-methyl-amino]pyrrolo[2,3-d]pyrimidin-7-yl]methyl 4-(tert-butoxycarbonylamino)butanoate (0.12 g, 0.221 mmol) in DCM (5 mL) was slowly added trifluoroacetic acid (0.5 mL) at ice-bath. The reaction mixture was allowed to cool to room temperature and then was stirred at room temperature for 16 hours. The solution was concentrated under reduced pressure to afford the title compound as a white solid (0.9 g, Yield: 95%) that was used without further purification. MS (m/z): [M+H]⁺ calcd for C₂₁H₂₉N₇O₃, 428.23; found, 428.1.

### Step 3: Preparation of conjugate between HA (Sodium hyaluronate) and [4-[[(3R,4R)-1-(2-cyanoacetyl)-4-methyl-3-piperidyl]-methyl-amino]pyrrolo[2,3-d]pyrimidin-7-yl]methyl 4-aminobutanoate

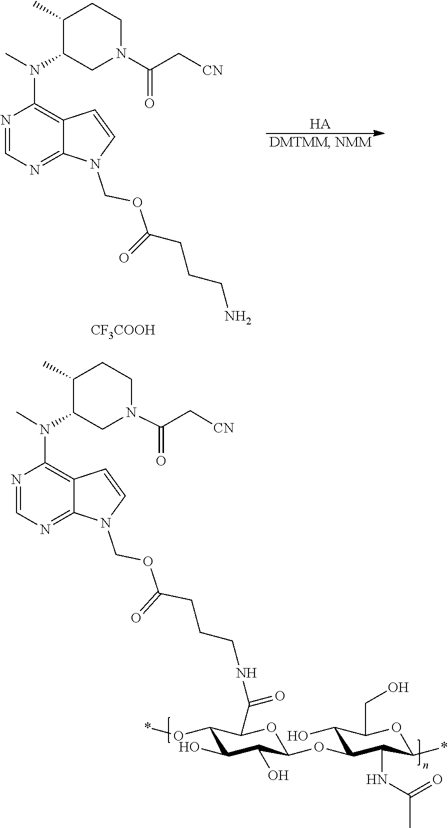

Sodium hyaluronate (127 mg, 0.31 mmol carboxylic acid, MW 50 kDa) was dissolved in 53 mL of deionized water in a 100 mL round-bottomed flask. To the solution was added [4-[[(3R,4R)-1-(2-cyanoacetyl)-4-methyl-3-piperidyl]-methyl-amino]pyrrolo[2,3-d]pyrimidin-7-yl]methyl 4-aminobutanoate trifluoroacetate (119 mg, 0.22 mmol) and 4-methylmorpholine (NMM, 22 mg, 0.22 mmol) at room temperature, causing the viscosity to increase temporarily. The reaction mixture was stirred at this temperature for 2 hours. To the solution was added 4-(4,6-dimethoxy-1,3,5-triazin-2-yl)-4-methylmorpholinium chloride (DMTMM, 85.7 mg, 0.31 mmol) was added and stirred for 72 hours at room temperature. NaCl (181 mg, 3.1 mmol) was then added to the reaction mixture, which was stirred for 1 hour and followed by the dropwise addition of acetone (300 mL) while stirring. The mixture was filtered. The filter cake was dissolved in 80 mL of deionized water and acetonitrile (V/V=3:1). NaCl (181 mg, 3.1 mmol) was then added to the reaction mixture, which was stirred for 1 hour and followed by the dropwise addition of acetone (200 mL) while stirring. The mixture was filtered. The filter cake was collected, washed with acetone and dried in vacuo to give the title compound as a white solid. 0.119 g, Yield: 48.7%, DSR: 24%; ¹H NMR (400 MHz, D₂O) δ ppm 8.23-7.99 (m, 0.24H), 7.41-7.09 (m, 0.24H), 6.83-6.63 (m, 0.24H), 6.18-5.89 (m, 0.48H), 4.59-4.16 (m, 2.24H), 4.24-2.66 (m, 12.64H), 2.50-2.18 (m, 0.72H), 2.04-1.48 (m, 3.72H), 1.27-1.18 (m, 0.24H), 1.12-0.76 (m, 0.72H).

With this step, reaction of Sodium hyaluronate (MW 2000 KDa) provided corresponding product (0.109 g, yield: 44.6%, DSR=22%).

### Example 70

### Preparation of conjugate between HA (Sodium hyaluronate) and methyl (3Z)-1-[[(1S)-5-amino-1-methoxycarbonyl-pentyl]carbamoyl]-3-[[4-[methyl-[2-(4-methylpiperazin-1-yl)acetyl]amino]

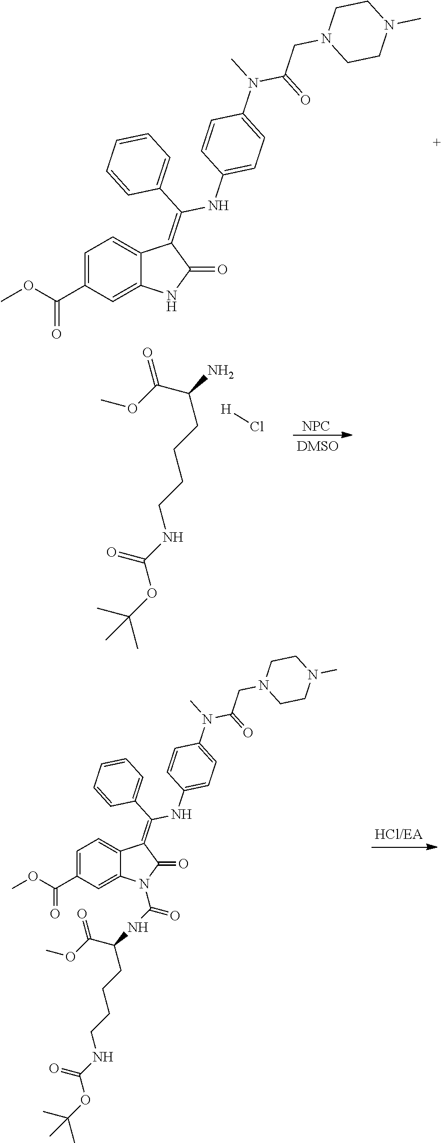

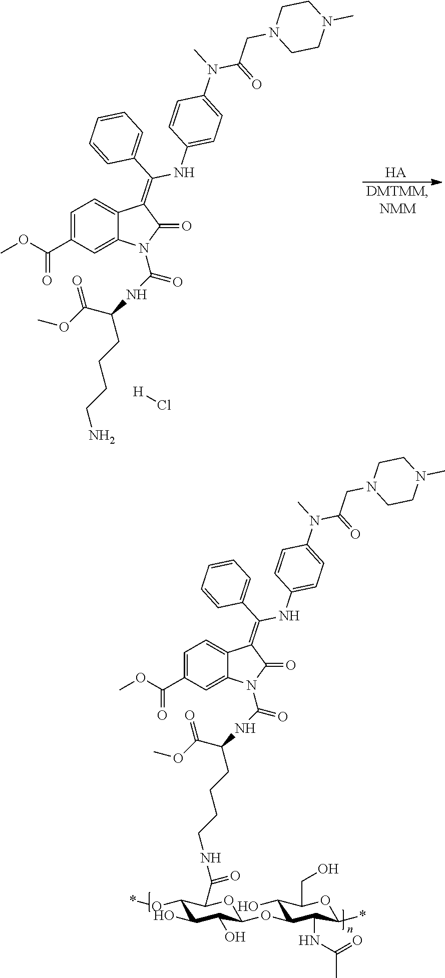

### Step 1: Preparation of methyl (3Z)-1-[[(1S)-5-(tert-butoxycarbonylamino)-1-methoxycarbonyl-pentyl]carbamoyl]-3-[[4-[methyl-[2-(4-methylpiperazin-1-yl)acetyl]amino]anilino]-phenyl-methylene]-2-oxo-indoline-6-carboxylate

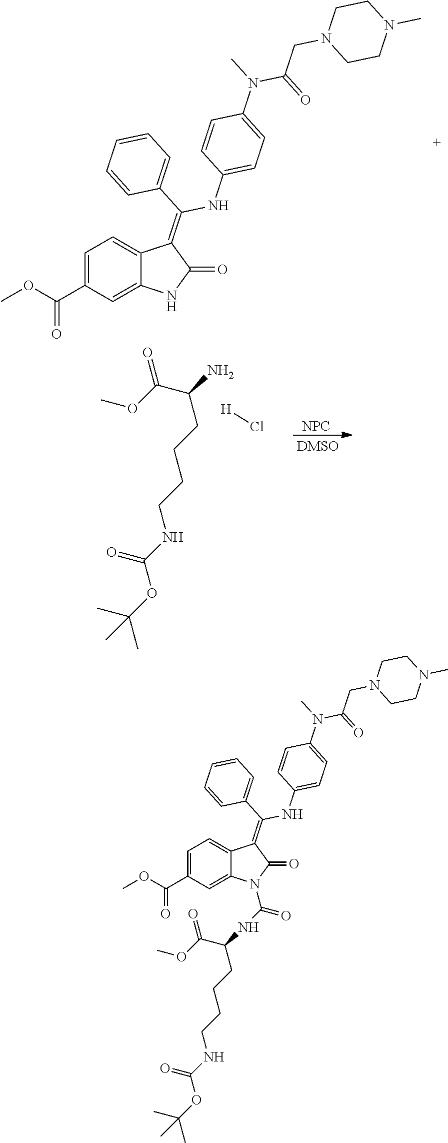

A mixture of methyl (3Z)-3-[[4-[methyl-[2-(4-methylpiperazin-1-yl)acetyl]amino]anilino]-phenyl-methylene]-2-oxo-indoline-6-carboxylate (1079 mg, 2 mmol) and bis(4-nitrophenyl) carbonate (668.8 mg, 2.2 mmol) in DMSO (30 mL) was stirred at room temperature for 7 hours. Then (S)-Methyl 6-amino-2-((tert-butoxycarbonyl)-amino)hexanoate (593.6 mg, 2 mmol) was added. The reaction mixture was stirred at room temperature for 16 hours. The reaction mixture was poured into water and lyophilization. The crude residue was purified by column chromatography (DCM:Methanol=5:1) to afford the title compound (1 g, Yield: 60.6%). MS (m/z): [M+H]⁺ calcd for C₄₄H₅₀N₇O₉, 826.40; found, 826.4. ¹H NMR (400 MHz, CDCl₃) δ ppm 12.03 (s, 1H), 9.60 (d, J=7.5 Hz, 1H), 8.93 (d, J=1.3 Hz, 1H), 7.63:1) to afford the title compound (1 g, hexanoate (593.6 mgxylate (1079 mg), 2.50-2.18 (m, 0.72H), dd, J=12.7, 7.4 Hz, 1H), 3.83 (d, J=16.3 Hz, 6H), 3.27-3.09 (m, 5H), 2.80 (s, 2H), 2.64-2.44 (m, 6H), 2.37 (s, 3H), 2.05-1.99 (m, 2H), 1.91 (dd, J=13.9, 8.9 Hz, 4H), 1.54 (dd, J=18.8, 7.6 Hz, 3H), 1.42 (s, 9H).

### Step 2: Preparation of methyl (3Z)-1-[[(1S)-5-amino-1-methoxycarbonyl-pentyl]carbamoyl]-3-[[4-[methyl-[2-(4-methylpiperazin-1-yl)acetyl]amino]anilino]-phenyl-methylene]-2-oxo-indoline-6-carboxylate hydrochloride

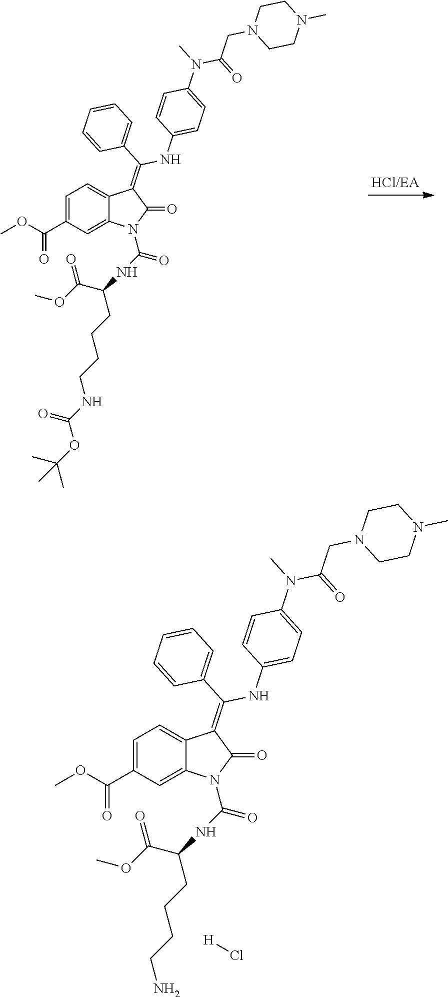

To a stirred solution of methyl (3Z)-1-[[(1S)-5-(tert-butoxycarbonylamino)-1-methoxycarbonyl-pentyl]carbamoyl]-3-[[4-[methyl-[2-(4-methylpiperazin-1-yl)acetyl]amino]anilino]-phenyl-methylene]-2-oxo-indoline-6-carboxylate (0.6 g, 0.686 mmol) in EtOAc (12 mL) was slowly added 4M HCl in ethyl acetate (commercially available) (2.4 mL) at ice-bath. The reaction mixture was allowed to cool to room temperature and then was stirred at room temperature for 16 hours. The solution was diluted with EtOAc and concentrated under reduced pressure to afford the title compound as a white solid (0.51 g, Yield: 97%) that was used without further purification. MS (m/z): [M+H]+ calcd for C₃₉H₄₇N₇O₇, 726.35; found, 726.2.

### Step 3: Preparation of conjugate between HA (Sodium hyaluronate) and methyl (3Z)-1-[[(1S)-5-amino-1-methoxycarbonyl-pentyl]carbamoyl]-3-[[4-[methyl-[2-(4-methylpiperazin-1-yl)acetyl]amino]anilino]-phenyl-methylene]-2-oxo-indoline-6-carboxylate

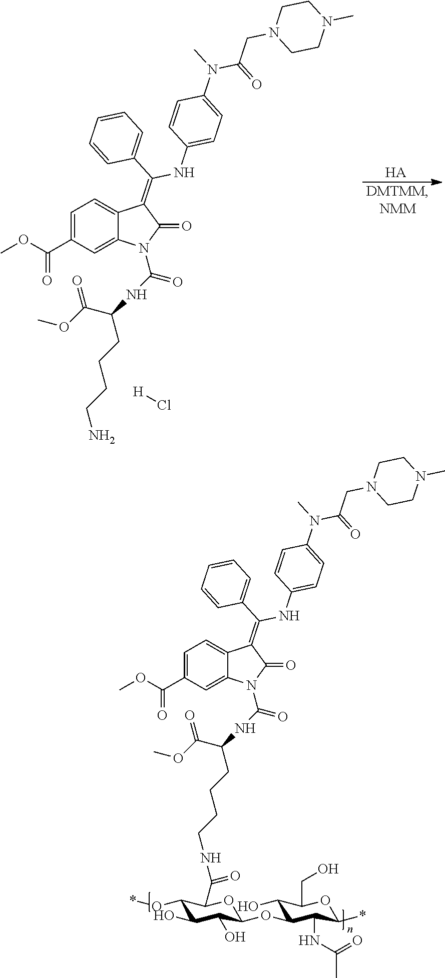

Sodium hyaluronate (121 mg, 0.3 mmol carboxylic acid, MW 50 KDa) was dissolved in 24.2 mL of deionized water in a 100 mL round-bottomed flask followed by the dropwise addition of 15.7 mL of acetonitrile while stirring. To the solution was added methyl (3Z)-1-[[(1S)-5-amino-1-methoxycarbonyl-pentyl]carbamoyl]-3-[[4-[methyl-[2-(4-methylpiperazin-1-yl)acetyl]amino]anilino]-phenyl-methylene]-2-oxo-indoline-6-carboxylate (160 mg, 0.21 mmol) and 4-methylmorpholine (NMM, 21 mg, 0.21 mmol) at room temperature, causing the viscosity to increase temporarily. The reaction mixture was stirred at this temperature for 2 hours. To the solution was added 4-(4,6-dimethoxy-1,3,5-triazin-2-yl)-4-methylmorpholinium chloride (DMTMM, 83 mg, 0.3 mmol) was added and stirred for 72 hours at room temperature. NaCl (123 mg, 2.1 mmol) was then added to the reaction mixture, which was stirred for 1 hour and followed by the dropwise addition of acetone (200 mL) while stirring. The mixture was filtered. The filter cake was dissolved in 80 mL of deionized water and acetonitrile (V/V=3:1). NaCl (123 mg, 2.1 mmol) was then added to the reaction mixture, which was stirred for 1 hour and followed by the dropwise addition of acetone (200 mL) while stirring. The mixture was filtered. The filter cake was collected, washed with acetone and dried in vacuo to give the title compound as a white solid. 0.142 g, Yield: 43.5%, DSR: 34%; ¹H NMR (400 MHz, D₂O) δ ppm 8.44-8.37 (m, 0.34H), 7.50-6.59 (m, 3.4H), 6.13-6.04 (m, 0.34H), 4.58-4.12 (m, 2.34H), 4.10-2.51 (m, 17.48H), 2.12-1.82 (m, 2.04H), 1.74-1.00 (m, 4.02H).

With this step, reaction of Sodium hyaluronate (MW 2000 KDa) provided corresponding product (0.13 g, yield: 39.9%).

### Example 71

### Preparation of conjugate between HA (Sodium hyaluronate) and methyl (3Z)-1-(hydrazinecarbonyl)-3-[[4-[methyl-[2-(4-methylpiperazin-1-yl)acetyl]amino]anilino]-phenyl-methylene]-2-oxo-indoline-6-carboxylate

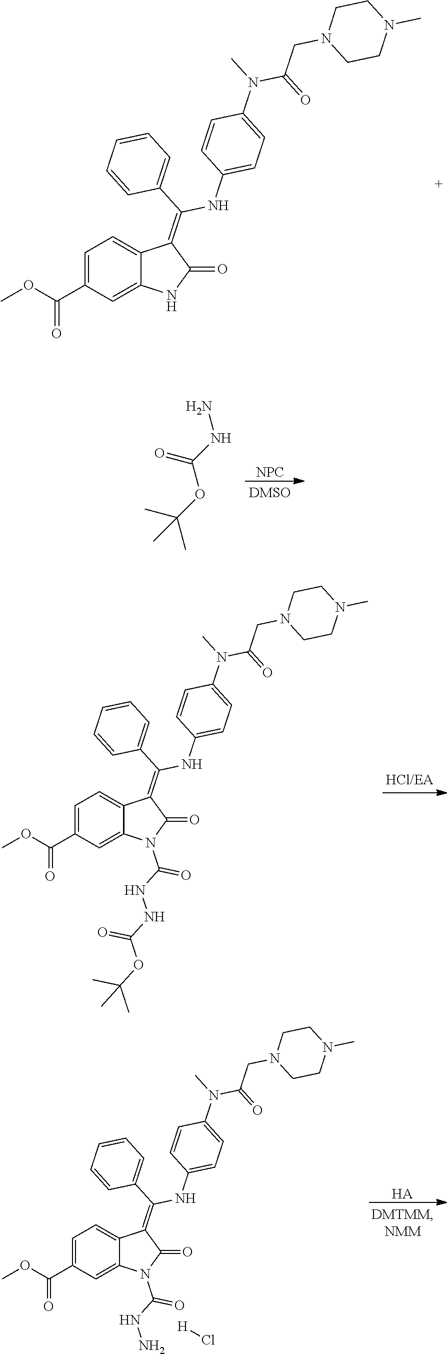

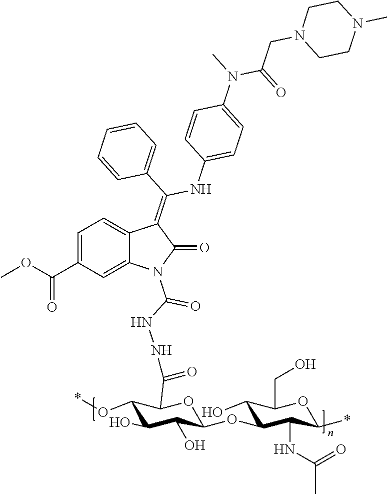

### Step 1: Preparation of methyl (3Z)-1-(hydrazinecarbonyl)-3-[[4-[methyl-[2-(4-methylpiperazin-1-yl)acetyl]amino]anilino]-phenyl-methylene]-2-oxo-indoline-6-carboxylate

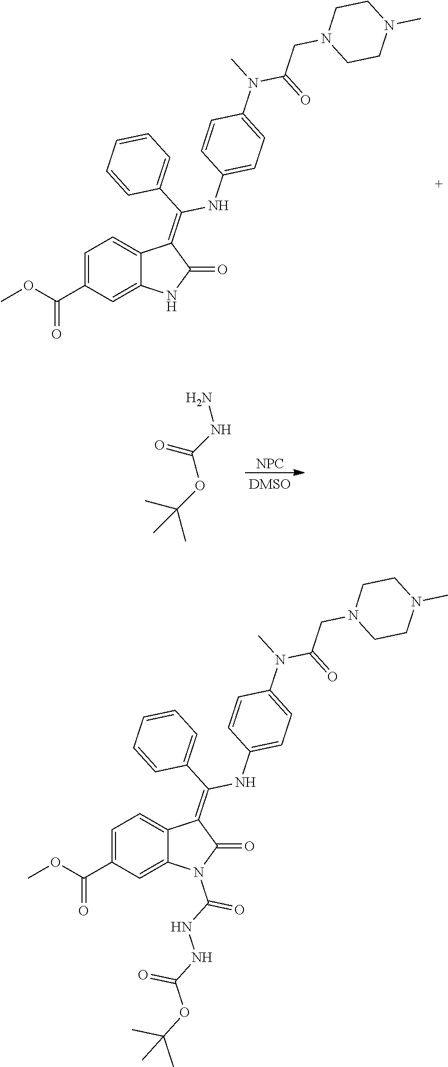

A mixture of methyl (3Z)-3-[[4-[methyl-[2-(4-methylpiperazin-1-yl)acetyl]amino]anilino]-phenyl-methylene]-2-oxo-indoline-6-carboxylate (1079 mg, 2 mmol) and bis(4-nitrophenyl) carbonate (668.8 mg, 2.2 mmol) in DMSO (30 mL) was stirred at room temperature for 7 hours. Then tert-Butyl hydrazinecarboxylate (264.3 mg, 2 mmol) was added. The reaction mixture was stirred at 50° C. for 16 hours. The reaction mixture was poured into water and extracted with EtOAc. The organic layer was dried over sodium sulfate, filtered and then concentrated under reduced pressure. The crude residue was purified by column chromatography (DCM:Methanol=8:1) to afford the title compound (0.5 g, Yield: 35.8%). MS (m/z): [M+H]+ calcd for C₃₇H₄₃N₇O₇, 698.32; found, 698.1. ¹H NMR (400 MHz, d-DMSO) δ ppm 11.79 (s, 1H), 10.29 (s, 1H), 9.26 (s, 1H), 8.69 (s, 1H), 7.76-7.33 (m, 6H), 7.19 (d, J=8.3 Hz, 2H), 7.06 (d, J=7.7 Hz, 2H), 5.76 (s, 1H), 3.80 (s, 3H), 3.08 (s, 3H), 2.74-2.53 (m, 2H), 2.19 (d, J=28.7 Hz, 10H), 1.46 (s, 9H).

### Step 2: Preparation of methyl (3Z)-1-(hydrazinecarbonyl)-3-[[4-[methyl-[2-(4-methylpiperazin-1-yl)acetyl]amino]anilino]-phenyl-methylene]-2-oxo-indoline-6-carboxylate hydrochloride

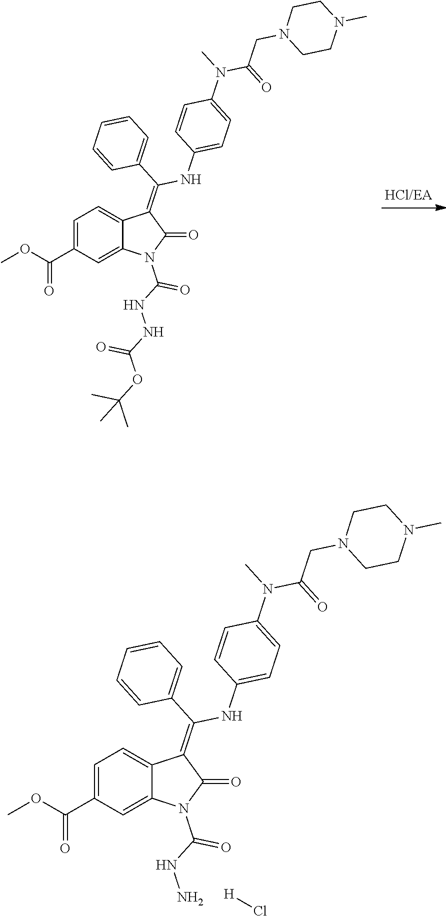

To a stirred solution of methyl (3Z)-1-(hydrazinecarbonyl)-3-[[4-[methyl-[2-(4-methylpiperazin-1-yl)acetyl]amino]anilino]-phenyl-methylene]-2-oxo-indoline-6-carboxylate (0.4 g, 0.57 mmol) in EtOAc (4 mL) was slowly added 4M HCl in ethyl acetate (commercially available) (1.6 mL) at ice-bath. The reaction mixture was allowed to cool to room temperature and then was stirred at room temperature for 16 hours. The solution was diluted with EtOAc and concentrated under reduced pressure to afford the title compound as a white solid (0.36 g, Yield: 99%) that was used without further purification. MS (m/z): [M+H]+ calcd for C₃₂H₃₅N₇O₅, 598.26; found, 598.1.

### Step 3: Preparation of conjugate between HA (Sodium hyaluronate) and methyl (3Z)-1-(hydrazinecarbonyl)-3-[[4-[methyl-[2-(4-methylpiperazin-1-yl)acetyl]amino]anilino]-phenyl-methylene]-2-oxo-indoline-6-carboxylate

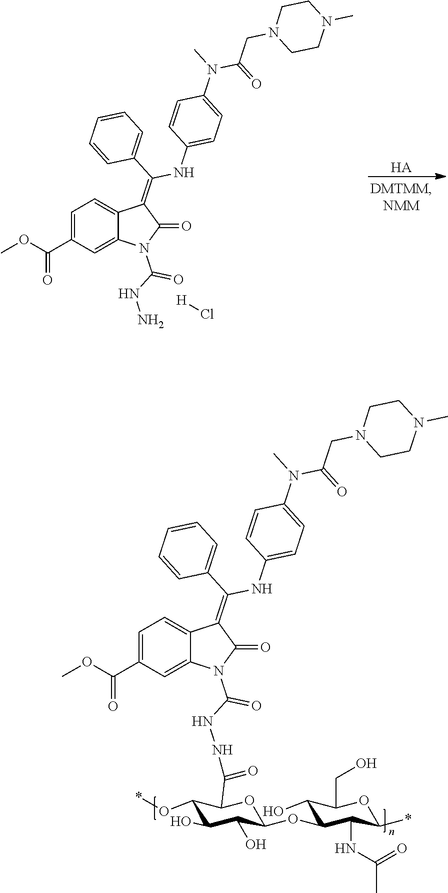

Sodium hyaluronate (136 mg, 0.337 mmol carboxylic acid, MW 50 KDa) was dissolved in 30 mL of deionized water in a 100 mL round-bottomed flask followed by the dropwise addition of 19.5 mL of acetonitrile while stirring. To the solution was added methyl (3Z)-1-(hydrazinecarbonyl)-3-[[4-[methyl-[2-(4-methylpiperazin-1-yl)acetyl]amino]anilino]-phenyl-methylene]-2-oxo-indoline-6-carboxylate hydrochloride (150 mg, 0.236 mmol) and 4-methylmorpholine (NMM, 23.9 mg, 0.236 mmol) at room temperature, causing the viscosity to increase temporarily. The reaction mixture was stirred at this temperature for 2 hours. To the solution was added 4-(4,6-dimethoxy-1,3,5-triazin-2-yl)-4-methylmorpholinium chloride (DMTMM, 93 mg, 0.337 mmol) was added and stirred for 72 hours at room temperature. NaCl (197 mg, 3.37 mmol) was then added to the reaction mixture, which was stirred for 1 hour and followed by the dropwise addition of acetone (200 mL) while stirring. The mixture was filtered. The filter cake was dissolved in 80 mL of deionized water and acetonitrile (V/V=3:1). NaCl (197 mg, 3.37 mmol) was then added to the reaction mixture, which was stirred for 1 hour and followed by the dropwise addition of acetone (200 mL) while stirring. The mixture was filtered. The filter cake was collected, washed with acetone, and dried in vacuo to give the title compound as a white solid. 0.153 g, Yield: 47.3%, DSR: 21%; ¹H NMR (400 MHz, D₂O) δ ppm 8.63-8.47 (m, 0.21H), 7.75-6.81 (m, 2.1H), 5.91-5.78 (m, 0.21H), 4.66-4.34 (m, 2H), 4.21-2.51 (m, 11.68H), 2.44-1.63 (m, 5.31H).

With this step, reaction of Sodium hyaluronate (MW 2000 KDa) provided corresponding product.

### Example 72

### Preparation of conjugate between HA (Sodium hyaluronate) and methyl (2R)-6-amino-2-[[2-[[2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]acetyl]amino]acetyl]amino]hexanoate

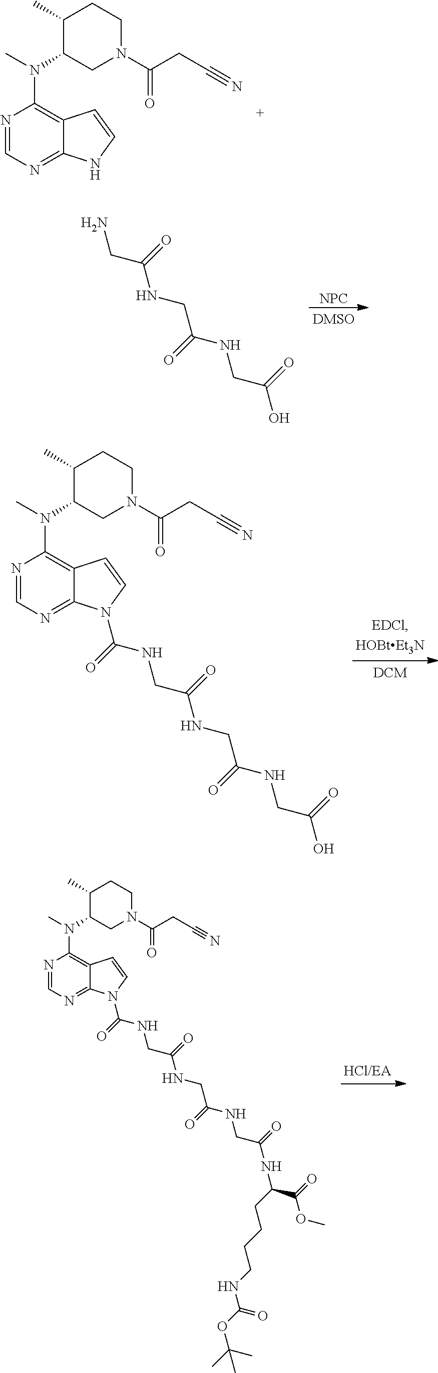 [SMILES: C[C@@H]1CCN(C(=O)CC#N)C[C@@H]1N(C)c1ncnc2[nH]ccc12 ; NCC(=O)NCC(=O)NCC(=O)O ; "NPC" ; C[C@@H]1CCN(C(=O)CC#N)C[C@@H]1N(C)c1ncnc2c1ccn2C(=O)NCC(=O)NCC(=O)NCC(=O)O ; COC(=O)[C@@H](CCCCNC(=O)OC(C)(C)C)NC(=O)CNC(=O)CNC(=O)CNC(=O)n1ccc2c(N(C)[C@H]3CN(C(=O)CC#N)CC[C@H]3C)ncnc21]

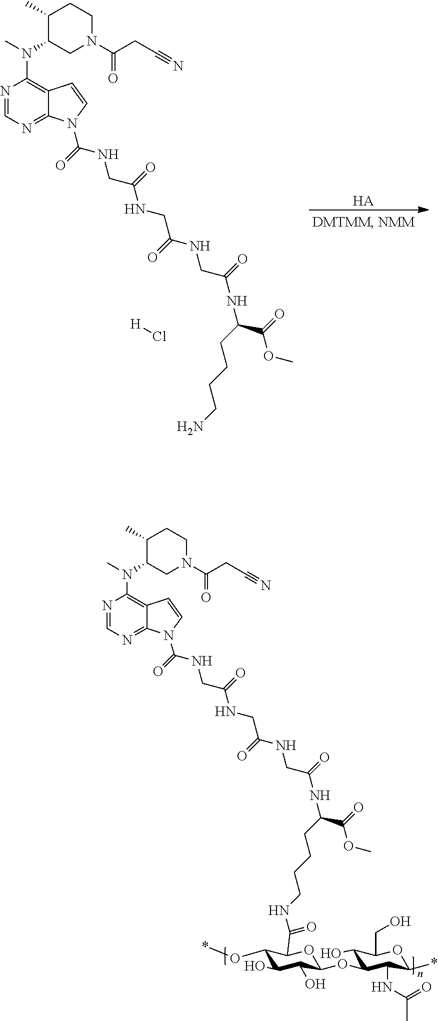

### Step 1: Preparation of 2-[[2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]acetyl]amino]acetic acid

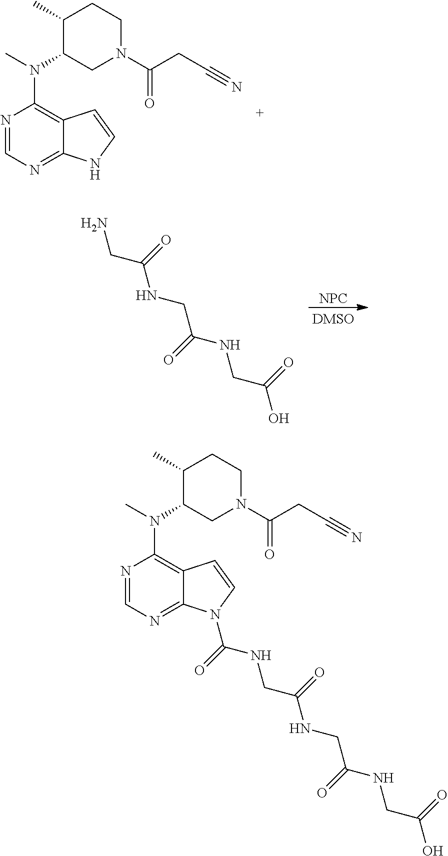 [SMILES: C[C@@H]1CCN(C(=O)CC#N)C[C@@H]1N(C)c1ncnc2[nH]ccc12 ; NCC(=O)NCC(=O)NCC(=O)O ; "NPC" ; C[C@@H]1CCN(C(=O)CC#N)C[C@@H]1N(C)c1ncnc2c1ccn2C(=O)NCC(=O)NCC(=O)NCC(=O)O]

A mixture of 3-[(3R,4R)-4-methyl-3-[methyl(7H-pyrrolo[2,3-d]pyrimidin-4-yl)amino]-1-piperidyl]-3-oxo-propanenitrile (1248 mg, 4 mmol) and bis(4-nitrophenyl) carbonate (1.337 mg, 4.4 mmol) in DMSO (30 mL) was stirred at room temperature for 7 hours. Then glycyl-glycyl-glycine (758.8 mg, 4 mmol) was added. The reaction mixture was stirred at 50° C. for 16 hours. The reaction mixture was poured into water and lyophilization. The crude residue was purified by column chromatography (DCM:Methanol=4:1) to afford the title compound (0.7 g, Yield: 33.2%). MS (m/z): [M+H]⁺ calcd for C₂₃H₂₉N₉O₆, 528.22; found, 528.1. ¹H NMR (400 MHz, d-DMSO) δ ppm 12.78-12.38 (m, 1H), 9.98 (t, J=5.3 Hz, 1H), 8.46 (d, J=5.7 Hz, 1H), 8.35 (d, J=5.7 Hz, 1H), 8.21 (s, 1H), 7.67 (d, J=4.0 Hz, 1H), 6.85 (s, 1H), 4.87 (s, 1H), 4.21-4.02 (m, 4H), 4.01-3.60 (m, 8H), 3.39 (d, J=16.8 Hz, 3H), 2.39 (d, J=6.4 Hz, 1H), 1.78 (d, J=47.7 Hz, 1H), 1.60 (d, J=8.7 Hz, 1H), 1.02 (d, J=7.1 Hz, 3H).

### Step 2: Preparation of methyl (2R)-6-(tert-butoxycarbonylamino)-2-[[2-[[2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]acetyl]amino]acetyl]amino]hexanoate

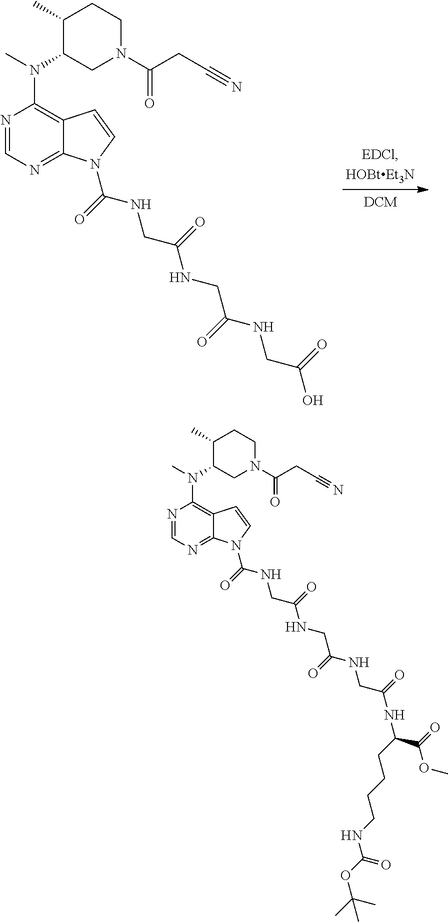 [SMILES: C[C@@H]1CCN(C(=O)CC#N)C[C@@H]1N(C)c1ncnc2c1ccn2C(=O)NCC(=O)NCC(=O)NCC(=O)O ; COC(=O)[C@@H](CCCCNC(=O)OC(C)(C)C)NC(=O)CNC(=O)CNC(=O)CNC(=O)n1ccc2c(N(C)[C@H]3CN(C(=O)CC#N)CC[C@H]3C)ncnc21]

2-[[2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]acetyl]amino]acetic acid (768 mg, 1.46 mmol), (S)-methyl 6-amino-2-((tert-butoxycarbonyl)-amino)hexanoate (454 mg, 1.53 mmol), 1-ethyl-3-(3-dimethylaminopropyl)carbodiimide (EDCI, 335 mg, 1.75 mmol) and 1-hydroxybenzotriazole (HOBt, 236 mg, 1.75 mmol) were dissolved in DCM (20 mL), DMF (2 mL) and triethylamine (368 mg, 3.64 mmol) was added. The reaction mixture was stirred at room temperature for 16 hours. The solution was diluted with DCM and washed with water and saturated brine solution. The organic layer was dried over sodium sulfate, filtered and then concentrated under reduced pressure. The crude residue was purified by column chromatography (Methanol/DCM=1: 150-1:80) to afford the title compound (0.66 g, Yield: 76.7%). MS (m/z): [M+H]⁺ calcd for C₃₅H₅₁N₁₁O₉, 770.38; found, 770.2. ¹H NMR (400 MHz, CDCl3) δ ppm 10.41 (s, 1H), 8.29 (d, J=8.0 Hz, 1H), 7.63 (dd, J=20.0, 3.8 Hz, 1H), 7.41 (s, 1H), 7.05-6.86 (m, 1H), 6.60 (dd, J=12.6, 4.0 Hz, 1H), 5.12 (s, 1H), 4.89 (s, 1H), 4.51 (s, 1H), 4.16 (d, J=36.3 Hz, 2H), 4.13-3.80 (m, 5H), 3.79-3.43 (m, 9H), 3.42-3.28 (m, 3H), 3.01 (d, J=26.8 Hz, 2H), 2.51 (d, J=6.2 Hz, 1H), 2.05-1.92 (m, 1H), 1.86-1.76 (m, 1H), 1.43 (s, 9H), 1.38-1.16 (m, 6H), 1.14-0.99 (m, 3H).

### Step 3: Preparation of methyl (2R)-6-amino-2-[[2-[[2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]acetyl]amino]acetyl]amino]hexanoate hydrochloride

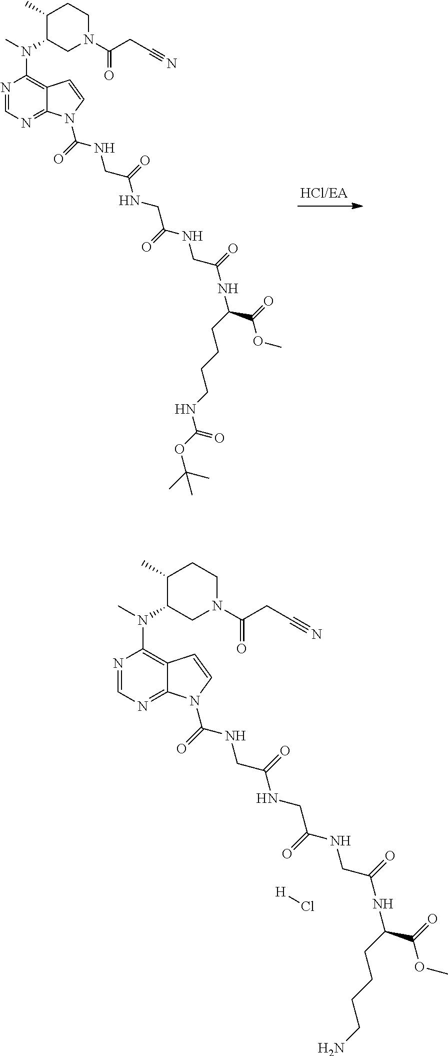 [SMILES: COC(=O)[C@@H](CCCCNC(=O)OC(C)(C)C)NC(=O)CNC(=O)CNC(=O)CNC(=O)n1ccc2c(N(C)[C@H]3CN(C(=O)CC#N)CC[C@H]3C)ncnc21 ; COC(=O)[C@@H](CCCCN)NC(=O)CNC(=O)CNC(=O)CNC(=O)n1ccc2c(N(C)[C@H]3CN(C(=O)CC#N)CC[C@H]3C)ncnc21 ; Cl]

To a stirred solution of methyl (2R)-6-(tert-butoxycarbonylamino)-2-[[2-[[2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]acetyl]amino]acetyl]amino]hexanoate (0.51 g, 0.663 mmol) in EtOAc (20 mL) was slowly added 4M HCl in ethyl acetate (commercially available) (8.5 mL) at ice-bath. The reaction mixture was allowed to cool to room temperature and then was stirred at room temperature for 16 hours. The solution was diluted with EtOAc and concentrated under reduced pressure to afford the title compound as a white solid (0.44 g, Yield: 99%) that was used without further purification. MS (m/z): [M+H]+ calcd for C₃₀H₄₃N₁₁O₇, 670.33; found, 670.1.

### Step 4: Preparation of conjugate between HA (Sodium hyaluronate) and methyl (2R)-6-amino-2-[[2-[[2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]acetyl]amino]acetyl]amino]hexanoate

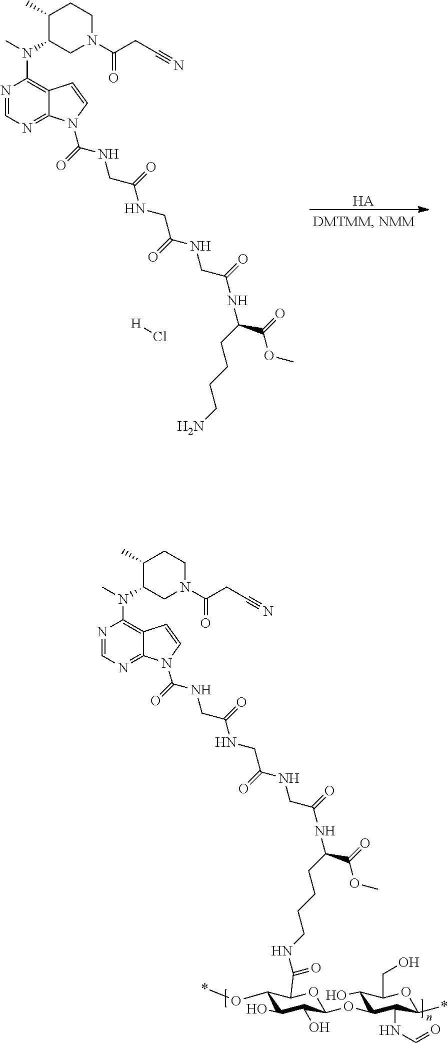

Sodium hyaluronate (245 mg, 0.607 mmol carboxylic acid, MW 50 KDa) was dissolved in 60 mL of deionized water in a 100 mL round-bottomed flask followed by the dropwise addition of 39 mL of acetonitrile while stirring. To the solution was added methyl (2R)-6-amino-2-[[2-[[2-[[2-[[4-[[(3R,4R)-1-(2-cyanoacetyl)-4-methyl-3-piperidyl]-methyl-amino]pyrrolo[2,3-d]pyrimidine-7-carbonyl]amino]acetyl]amino]acetyl]amino]acetyl]amino]hexanoate hydrochloride (300 mg, 0.425 mmol) and 4-methylmorpholine (NMM, 43 mg, 0.425 mmol) at room temperature, causing the viscosity to increase temporarily. The reaction mixture was stirred at this temperature for 2 hours. To the solution was added 4-(4,6-dimethoxy-1,3,5-triazin-2-yl)-4-methylmorpholinium chloride (DMTMM, 168 mg, 0.607 mmol) was added and stirred for 72 hours at room temperature. NaCl (355 mg, 6.07 mmol) was then added to the reaction mixture, which was stirred for 1 hour and followed by the dropwise addition of acetone (400 mL) while stirring. The mixture was filtered. The filter cake was dissolved in 80 mL of deionized water and acetonitrile (V/V=3:1). NaCl (355 mg, 6.07 mmol) was then added to the reaction mixture, which was stirred for 1 hour and followed by the dropwise addition of acetone (400 mL) while stirring. The mixture was filtered. The filter cake was collected, washed with acetone and dried in vacuo to give the title compound as a white solid. 0.25 g, Yield: 41%, DSR: 15%; ¹H NMR (400 MHz, D₂O) δ ppm 8.26-8.13 (m, 0.15H), 7.60-7.47 (m, 0.15H), 6.80-6.66 (m, 0.15H), 4.66-4.28 (m, 2.15H), 4.24-2.69 (m, 13.15H), 2.41-2.24 (m, 0.15H), 2.04-1.42 (m, 3.3H), 1.37-0.89 (m, 1.35H).

With this step, reaction of Sodium hyaluronate (MW 2000 KDa) provided corresponding product (0.26 g, yield: 42.6%, DSR=14%). ¹H NMR (400 MHz, D₂O) δ ppm 8.26-8.13 (m, 0.14H), 7.59-7.48 (m, 0.14H), 6.79-6.66 (m, 0.14H), 4.66-4.28 (m, 2.14H), 4.26-2.66 (m, 12.94H), 2.40-2.24 (m, 0.14H), 2.03-1.42 (m, 3.28H), 1.35-0.89 (m, 1.26H).

### Example 73

### Preparation of conjugate of 2-azaspiro[3.3]heptan-6-yl-4-(((3R,4R)-1-(2-cyanoacetyl)-4-methylpiperidin-3-yl)(methyl)amino)-7H-pyrrolo[2,3-d]pyrimidine-7-carboxylate and HA

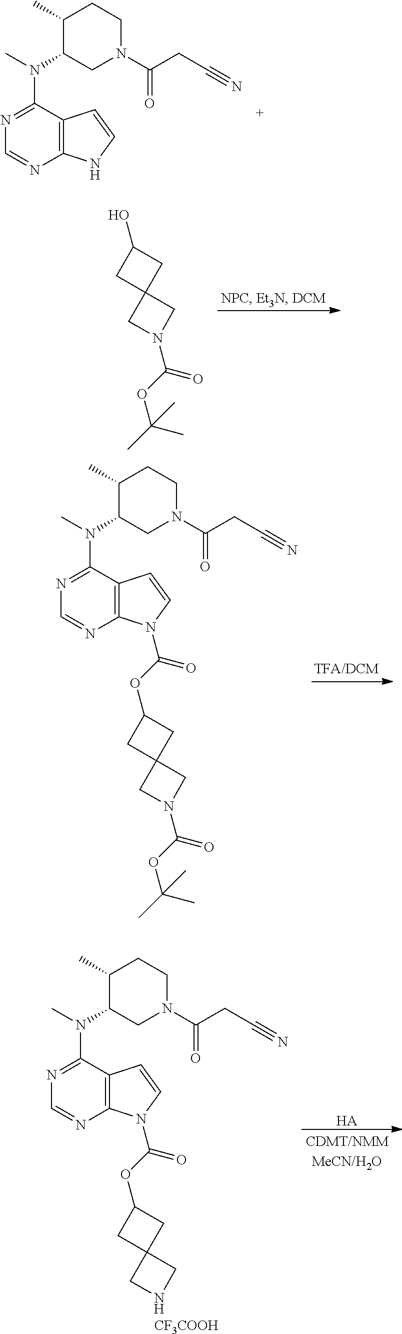

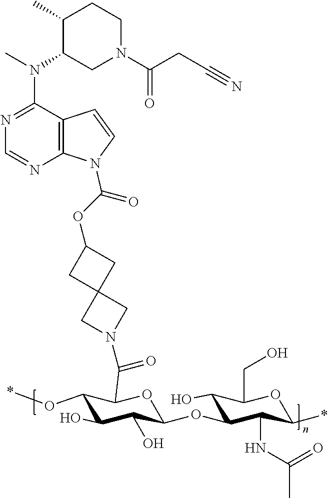

### Step 1: Preparation of 2-(tert-butoxycarbonyl)-2-azaspiro[3.3]heptan-6-yl 4-(((3R,4R)-1-(2-cyanoacetyl)-4-methylpiperidin-3-yl)(methyl)amino)-7H-pyrrolo[2,3-d]pyrimidine-7-carboxylate

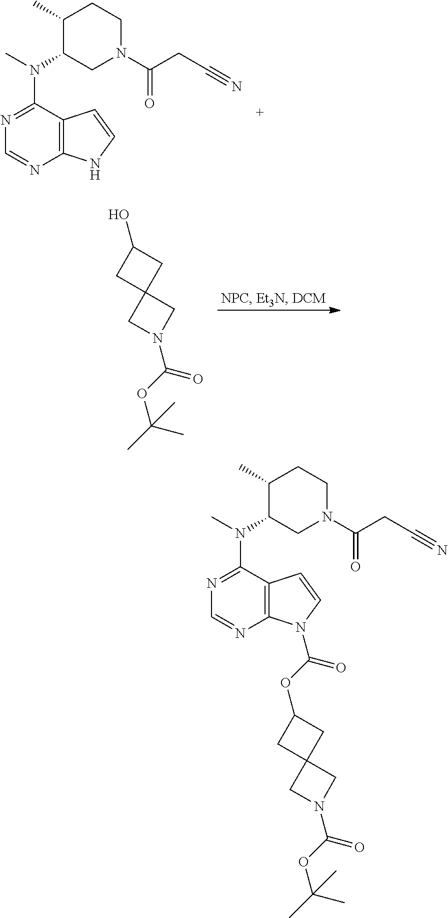

To a stirred mixture of 3-[(3R,4R)-4-methyl-3-[methyl(7H-pyrrolo[2,3-d]pyrimidin-4-yl)amino]-1-piperidyl]-3-oxo-propanenitrile (812 mg, 2.6 mmol) and bis(4-nitrophenyl) carbonate (870 mg, 2.86 mmol) in DCM (60 mL) was added triethylamine (1.19 mL, 8.58 mmol). The reaction mixture was heated to 45° C. and stirred at this temperature for 6 hours. Then the reaction mixture was cooled to room temperature. Then tert-butyl 6-hydroxy-2-azaspiro[3.3]heptane-2-carboxylate (832 mg, 3.9 mmol) was added. The reaction mixture was stirred at room temperature for 16 hours. The solution was diluted with DCM and washed with saturated NaHCO₃ solution, water and saturated brine solution. The organic layer was dried over sodium sulfate, filtered and then concentrated under reduced pressure. The crude residue was purified by column chromatography (EtOAc:Hexane=5:1-1:1) to afford the title compound (0.58 g, Yield: 40%). MS (m/z): [M+H]+ calcd for C₂₈H₃₇N₇O₅, 552.65; found: 552.2.

### Step 2: Preparation of 2-azaspiro[3.3]heptan-6-yl-4-(((3R,4R)-1-(2-cyanoacetyl)-4-methylpiperidin-3-yl)(methyl)amino)-7H-pyrrolo[2,3-d]pyrimidine-7-carboxylate trifluoroacetic acid

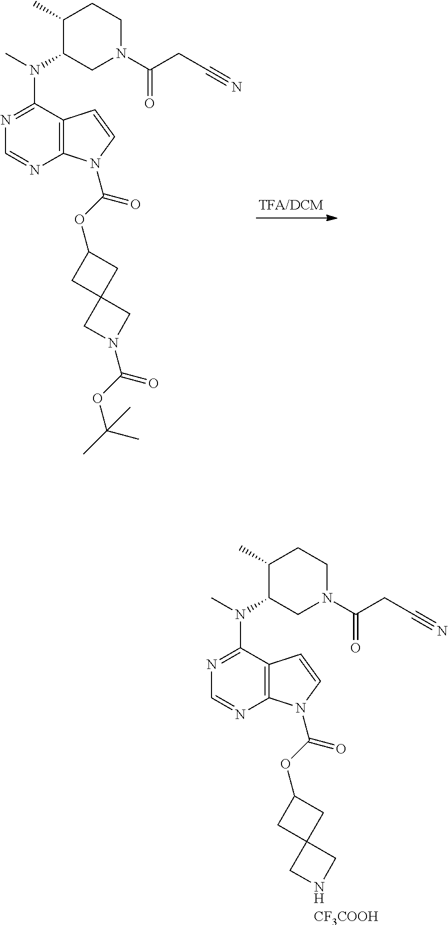 [SMILES: C[C@@H]1CCN(C(=O)CC#N)C[C@@H]1N(C)c1ncnc2c1ccn2C(=O)OC1CC2(C1)CN(C(=O)OC(C)(C)C)C2 ; C[C@@H]1CCN(C(=O)CC#N)C[C@@H]1N(C)c1ncnc2c1ccn2C(=O)OC1CC2(CNC2)C1 ; "CF3COOH"]

To a stirred solution of 2-(tert-butoxycarbonyl)-2-azaspiro[3.3]heptan-6-yl 4-(((3R,4R)-1-(2-cyanoacetyl)-4-methylpiperidin-3-yl)(methyl)amino)-7H-pyrrolo[2,3-d]pyrimidine-7-carboxylate (166 mg, 0.3 mmol) in DCM (16.5 mL) was slowly added CF₃COOH (3.3 mL) at ice-bath. The reaction mixture was warmed to room temperature and then was stirred at room temperature for 3 hours. The solution was diluted with DCM and concentrated under reduced pressure to afford the title compound as a brown oil (0.2 g, Yield: 100%) that was used to next step without further purification. MS (m/z): [M+H]+ calcd for C₂₃H₂₉N₇O₃, 452.53; found: 452.3.

### Step 3: Preparation of conjugate of 2-azaspiro[3.3]heptan-6-yl-4-(((3R,4R)-1-(2-cyanoacetyl)-4-methylpiperidin-3-yl)(methyl)amino)-7H-pyrrolo[2,3-d]pyrimidine-7-carboxylate and HA

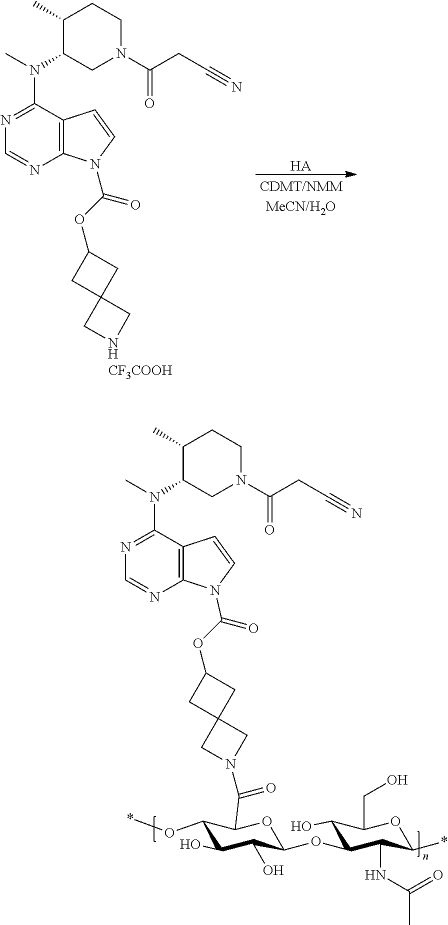

To a solution of Hyaluronic acid (161 mg, 0.4 mmol) was dissolved in 32 mL of deionized water and 20 mL of acetonitrile was added 4-methylmorpholine (NMM, 28 mg, 0.28 mmol) and the solution was then cooled to 0° C. 2-chloro-4,6-dimethoxy-1,3,5-triazine (166 mg, 0.6 mmol) was added and stirred at room temperature for 1 hour. Then A solution of 2-azaspiro[3.3]heptan-6-yl 4-(((3R,4R)-1-(2-cyanoacetyl)-4-methylpiperidin-3-yl)(methyl)amino)-7H-pyrrolo[2,3-d]pyrimidine-7-carboxylatehydrochloride (157 mg, 0.28 mmol) in water (5 ml) was added and stirred for 72 hours at room temperature. NaCl (234 mg, 4 mmol) was then added to the reaction mixture, which was stirred for 1 hour and followed by the dropwise addition of acetone (300 mL) while stirring. The mixture was filtered. The filter cake was washed with acetone, and dried under vacuum to give the title compound as a white solid. (Sodium hyaluronate MW 2000 KDa, 0.14 g, Yield: 61.5%, DS: 3%; ¹H NMR (400 MHz, D₂O) δ ppm 8.27-8.18 (m, 0.03H), 7.63-7.51 (m, 0.03H), 6.90-6.77 (m, 0.03H), 5.29-5.16 (m, 0.03H), 4.57-4.28 (m, 2.03H), 4.11-2.87 (m, 10.39H), 2.64-2.47 (m, 0.12H), 2.30-1.46 (m, 3.06H), 1.07-0.97 (m, 0.09H).

### Example 74

### Preparation of conjugate of 3-aminocyclobutyl 4-(((3R,4R)-1-(2-cyanoacetyl)-4-methylpiperidin-3-yl)(methyl)amino)-7H-pyrrolo[2,3-d]pyrimidine-7-carboxylate and HA

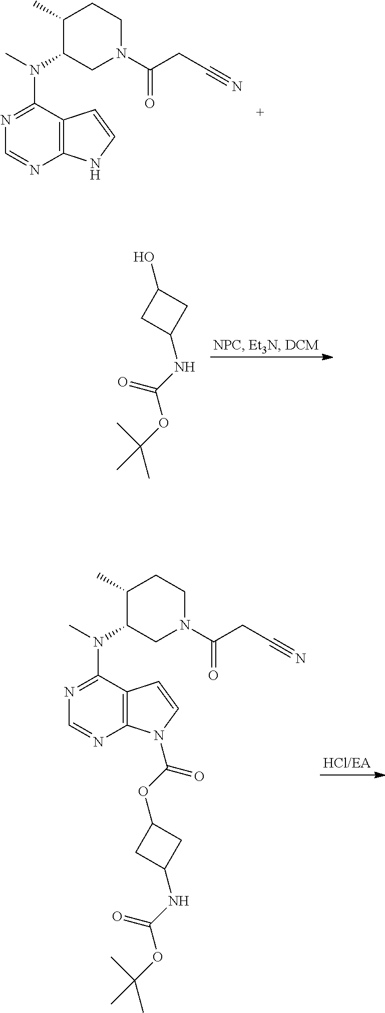

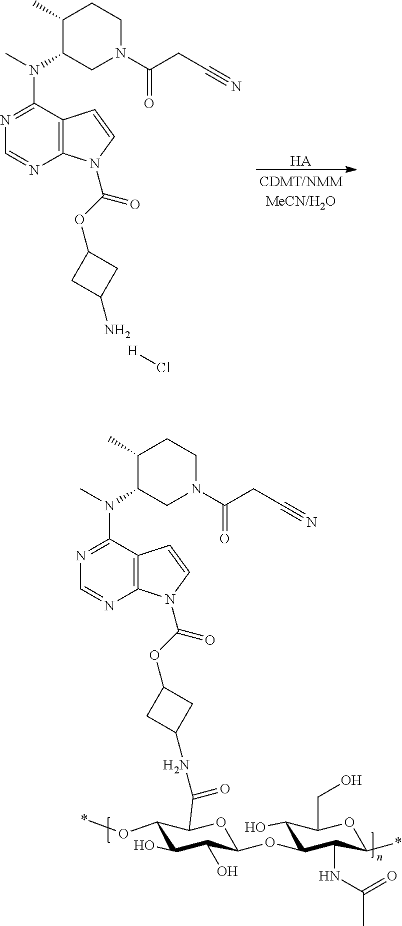

### Step 1: Preparation of 3-((tert-butoxycarbonyl)amino)cyclobutyl 4-(((3R,4R)-1-(2-cyanoacetyl)-4-methylpiperidin-3-yl)(methyl)amino)-7H-pyrrolo[2,3-d]pyrimidine-7-carboxylate

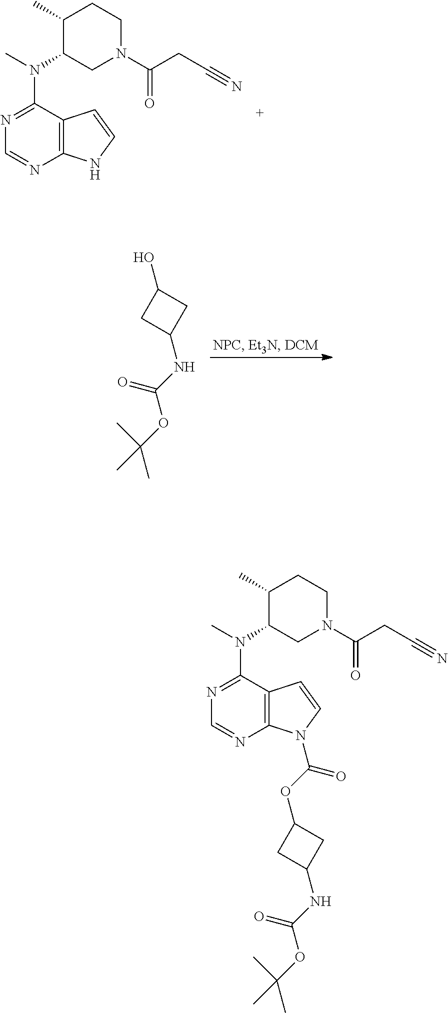

To a stirred mixture of 3-[(3R,4R)-4-methyl-3-[methyl(7H-pyrrolo[2,3-d]pyrimidin-4-yl)amino]-1-piperidyl]-3-oxo-propanenitrile (1249.5 mg, 4 mmol) and bis(4-nitrophenyl) carbonate (1460 mg, 4.8 mmol) in DCM (120 mL) was added triethylamine (1012 mg, 1.39 mL, 10 mmol). The reaction mixture was heated to 45° C. and stirred at this temperature for 6 hours. Then the reaction mixture was cooled to room temperature. Then tert-butyl (3-hydroxycyclobutyl)carbamate (1123 mg, 6 mmol) was added. The reaction mixture was stirred at room temperature for 22 hours. The solution was diluted with DCM and washed with saturated NaHCO₃ solution, water and saturated brine solution. The organic layer was dried over sodium sulfate, filtered and then concentrated under reduced pressure. The crude residue was purified by column chromatography (EtOAc:Hexane=1: 10-1:5) to afford the title compound (1.5 g, Yield: 71.4%). MS (m/z): [M+H]+ calcd for C₂₆H₃₅N₇O₅, 526.61; found: 526.2. ¹H NMR (400 MHz, Chloroform-d) δ ppm 8.47 (d, J=9.3 Hz, 1H), 7.44 (dd, J=11.3, 4.2 Hz, 1H), 6.64 (d, J=4.2 Hz, 1H), 5.19 (d, J=33.2 Hz, 2H), 5.05 (t, J=6.8 Hz, 1H), 4.04 (dd, J=13.2, 4.6 Hz, 1H), 3.82 (dd, J=13.7, 7.4 Hz, 1H), 3.69-3.57 (m, 1H), 3.56-3.44 (m, 2H), 3.37 (d, J=18.8 Hz, 3H), 3.09-2.95 (m, 2H), 2.50 (dt, J=14.1, 6.3 Hz, 1H), 2.18 (d, J=10.0 Hz, 2H), 2.01-1.86 (m, 1H), 1.83-1.69 (m, 1H), 1.68-1.53 (m, 2H), 1.45 (s, 9H), 1.08 (dd, J=15.2, 7.1 Hz, 3H).

### Step 2: Preparation of 3-aminocyclobutyl-4-(((3R,4R)-1-(2-cyanoacetyl)-4-methylpiperidin-3-yl)(methyl)amino)-7H-pyrrolo[2,3-d]pyrimidine-7-carboxylate hydrochloride

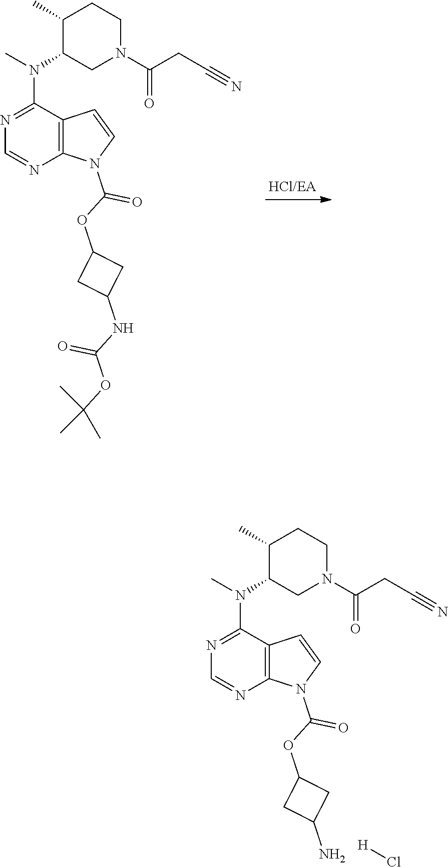

To a solution of 3-((tert-butoxycarbonyl)amino)cyclobutyl 4-(((3R,4R)-1-(2-cyanoacetyl)-4-methylpiperidin-3-yl)(methyl)amino)-7H-pyrrolo[2,3-d]pyrimidine-7-carboxylate (0.525 g, 1 mmol) in ethyl acetate (20 mL) was added 4M HCl in ethyl acetate solution (4 mL, 16 mmol) dropwisely under N₂ at 0° C. The resulting reaction mixture was stirred at 0° C. for 30 min and then stirred at room temperature for 20 hours. The solution was diluted with ethyl acetate (20 mL) and concentrated under reduced pressure to afford the title compound as white solid (0.46 g, Yield: 99.6%). MS (m/z): [M+H]+ calcd for C₂₁H₂₇N₇O₃, 426.49; found: 426.2.

### Step 3: Preparation of conjugate of 3-aminocyclobutyl 4-(((3R,4R)-1-(2-cyanoacetyl)-4-methylpiperidin-3-yl)(methyl)amino)-7H-pyrrolo[2,3-d]pyrimidine-7-carboxylate and HA

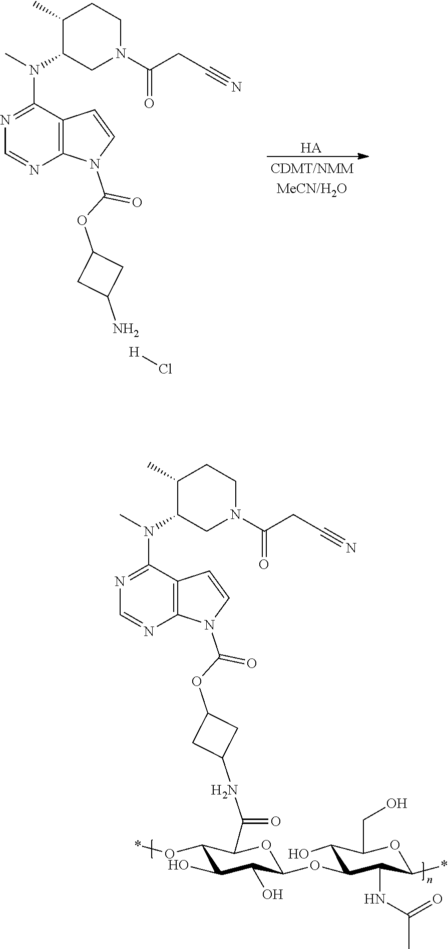

To a solution of Hyaluronic acid (201 mg, 0.5 mmol) in 40 mL of deionized water and 20 mL of acetonitrile was added 4-methylmorpholine (NMM, 50 mg, 0.5 mmol) and the solution was then cooled to 0° C. 2-Chloro-4,6-dimethoxy-1,3,5-triazine (277.5 mg, 1 mmol) was added and then stirred at room temperature for 1 hour. 3-aminocyclobutyl 4-(((3R,4R)-1-(2-cyanoacetyl)-4-methylpiperidin-3-yl)(methyl)amino)-7H-pyrrolo[2,3-d]pyrimidine-7-carboxylatehydrochloride (231 mg, 0.5 mmol) was added and then stirred for 72 hours at room temperature. NaCl (439 mg, 7.5 mmol) was then added to the reaction mixture and stirred for 1 hour and followed by the dropwise addition of acetone (300 mL) while stirring. The mixture was filtered. The filter cake was washed with acetone, and dried under vacuum to give the title compound as a white solid. (Sodium hyaluronate MW 50 KDa, 0.31 g Yield: 78.8%, DS: 33%); ¹H NMR (400 MHz, Deuterium Oxide) δ ppm 8.25-8.13 (m, 0.33H), 7.60-7.47 (m, 0.33H), 6.88-6.74 (m, 0.33H), 5.12-4.95 (m, 0.66H), 4.56-4.31 (m, 2H), 4.15-2.88 (m, 13.3H), 2.76-2.61 (m, 0.33H), 2.43-2.19 (m, 0.66H), 2.05-1.41 (m, 4.32H), 1.09-0.80 (m, 0.99H).

(Sodium hyaluronate MW 500 KDa, 0.22 g, Yield: 55.9%, DS: 41%), ¹H NMR (400 MHz, Deuterium Oxide) δ ppm 8.19-8.04 (m, 0.41H), 7.54-7.38 (m, 0.41H), 6.85-6.61 (m, 0.41H), 5.10-4.95 (m, 0.82H), 4.56-4.20 (m, 2H), 4.06-2.70 (m, 14.1H), 2.69-2.49 (m, 0.41H), 2.40-2.15 (m, 0.82H), 2.10-1.44 (m, 4.64H), 1.08-0.68 (m, 1.23H).

(Sodium hyaluronate MW 2000 KDa, 0.2 g, Yield: 50.8%, DS: 49%); ¹H NMR (400 MHz, Deuterium Oxide) δ ppm 8.20-7.89 (m, 0.49H), 7.60-6.98 (m, 0.49H), 6.61-6.54 (m, 0.49H), 5.22-4.87 (m, 0.98H), 4.64-4.26 (m, 2H), 4.16-2.80 (m, 14.9H), 2.78-2.63 (m, 0.49H), 2.48-2.24 (m, 0.98H), 2.07-1.47 (m, 4.96H), 1.11-0.76 (m, 1.47H).

### Example 75

### Preparation of conjugate of azepan-4-yl 4-(((3R,4R)-1-(2-cyanoacetyl)-4-methylpiperidin-3-yl)(methyl)amino)-7H-pyrrolo[2,3-d]pyrimidine-7-carboxylate and HA

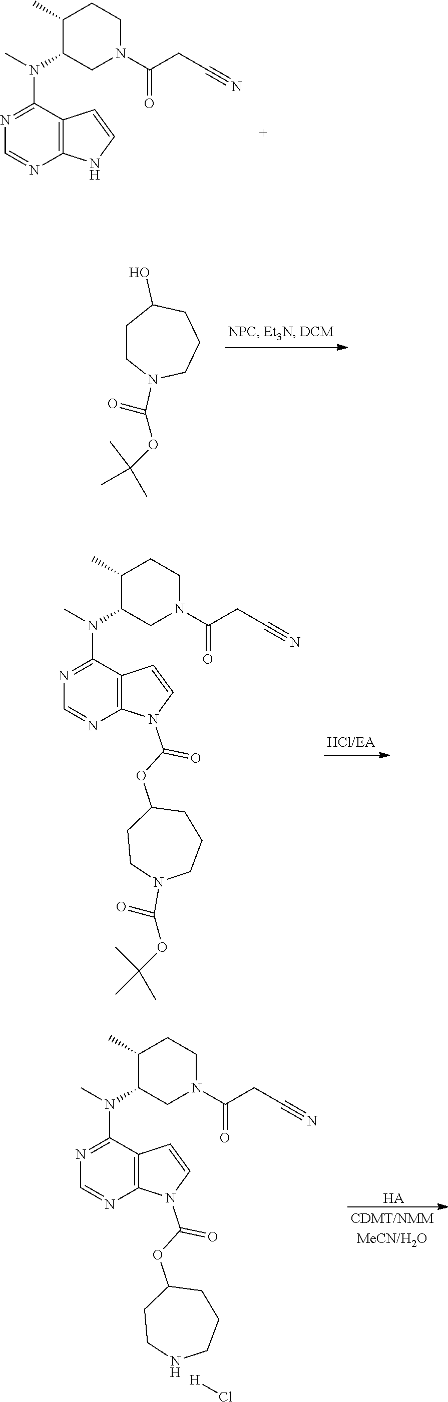

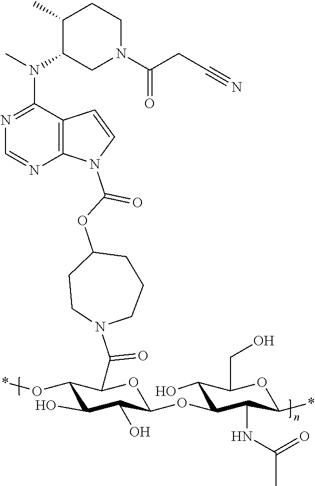

### Step 1: Preparation of 1-(tert-butoxycarbonyl)azepan-4-yl 4-(((3R,4R)-1-(2-cyanoacetyl)-4-methylpiperidin-3-yl)(methyl)amino)-7H-pyrrolo[2,3-d]pyrimidine-7-carboxylate

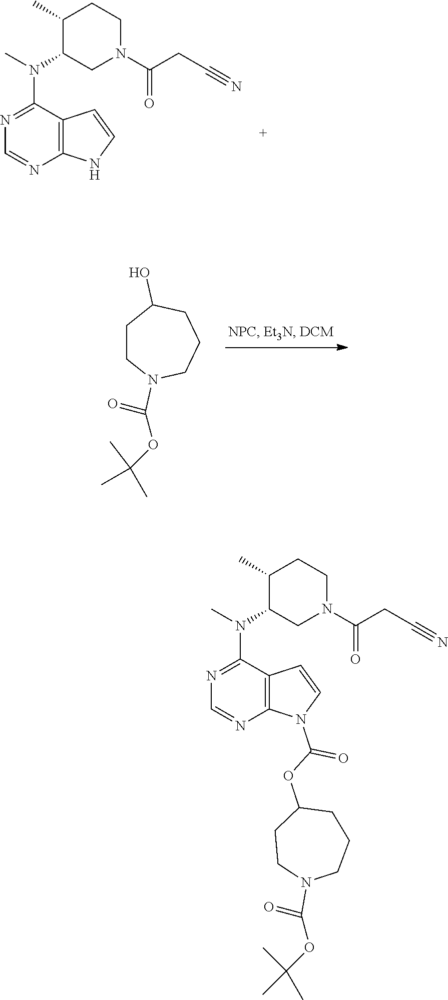 [SMILES: C[C@@H]1CCN(C(=O)CC#N)C[C@@H]1N(C)c1ncnc2[nH]ccc12 ; CC(C)(C)OC(=O)N1CCCC(O)CC1 ; C[C@@H]1CCN(C(=O)CC#N)C[C@@H]1N(C)c1ncnc2c1ccn2C(=O)OC1CCCN(C(=O)OC(C)(C)C)CC1]

To a stirred mixture of 3-[(3R,4R)-4-methyl-3-[methyl(7H-pyrrolo[2,3-d]pyrimidin-4-yl)amino]-1-piperidyl]-3-oxo-propanenitrile (937 mg, 3 mmol) and bis(4-nitrophenyl) carbonate (1094 mg, 3.6 mmol) in DCM (30 mL) was added triethylamine (2.78 mL, 20 mmol). The reaction mixture was heated to 40° C. and stirred at this temperature for 6 hours. Then the reaction mixture was cooled to room temperature. Then tert-butyl 4-hydroxyazepane-1-carboxylate (775 mg, 3.6 mmol) was added. The reaction mixture was stirred at room temperature for 22 hours. The solution was diluted with DCM and washed with saturated NaHCO₃ solution, water and saturated brine solution. The organic layer was dried over sodium sulfate, filtered and then concentrated under reduced pressure. The crude residue was purified by column chromatography (EtOAc:Hexane=1:1-4:1) to afford the title compound (0.93 g, Yield: 56%). MS (m/z): [M+H]+ calcd for C₂₈H₃₉N₇O₅, 554.66; found: 554.2. ¹H NMR (400 MHz, Chloroform-d) δ ppm 8.45 (d, J=8.3 Hz, 1H), 7.44 (dd, J=12.1, 4.1 Hz, 1H), 6.62 (d, J=4.0 Hz, 1H), 5.28 (s, 1H), 5.14 (s, 1H), 4.09-3.75 (m, 2H), 3.69-3.28 (m, 11H), 2.59-2.37 (m, 1H), 2.14-1.84 (m, 6H), 1.86-1.71 (m, 2H), 1.48 (s, 9H), 1.08 (dd, J=15.2, 7.1 Hz, 3H).

### Step 2: Preparation of azepan-4-yl 4-(((3R,4R)-1-(2-cyanoacetyl)-4-methylpiperidin-3-yl)(methyl)amino)-7H-pyrrolo[2,3-d]pyrimidine-7-carboxylate hydrochloride

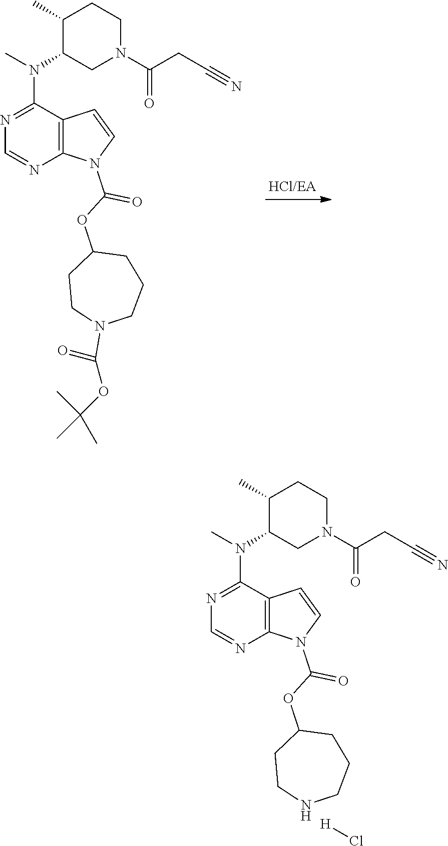

To a solution of 1-(tert-butoxycarbonyl)azepan-4-yl 4-(((3R,4R)-1-(2-cyanoacetyl)-4-methylpiperidin-3-yl)(methyl)amino)-7H-pyrrolo[2,3-d]pyrimidine-7-carboxylate (0.63 g, 1.14 mmol, 1 eq) in ethyl acetate (12 mL) was added 4M HCl in ethyl acetate solution (4 mL, 16 mmol) dropwisely under N₂ at 0° C. The resulting reaction mixture was stirred at 0° C. for 30 min and then stirred at room temperature for 16 hours. The solution was diluted with ethyl acetate (20 mL) and concentrated under reduced pressure to afford the title compound as a white solid (0.55 g, Yield: 98.5%) that was used without further purification. MS (m/z): [M+H]+ calcd for C₂₃H₃₁N₇O₃, 454.55; found: 454.2.

### Step 3: Preparation of conjugate of azepan-4-yl 4-(((3R,4R)-1-(2-cyanoacetyl)-4-methylpiperidin-3-yl)(methyl)amino)-7H-pyrrolo[2,3-d]pyrimidine-7-carboxylate and HA

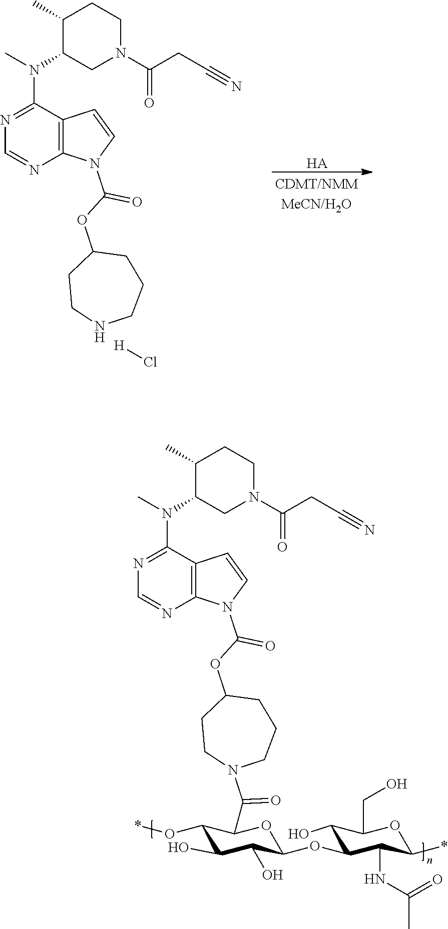

To a solution of Hyaluronic acid (201 mg, 0.5 mmol carboxylic acid) in 40 mL of deionized water and 20 mL of acetonitrile was added 4-methylmorpholine (NMM, 50 mg, 0.5 mmol), and the solution was then cooled to 0° C. 2-Chloro-4,6-dimethoxy-1,3,5-triazine (207.5 mg, 0.75 mmol) was added and stirred at room temperature for 1 hour. The solution was mixed with azepan-4-yl 4-(((3R,4R)-1-(2-cyanoacetyl)-4-methylpiperidin-3-yl)(methyl)amino)-7H-pyrrolo[2,3-d]pyrimidine-7-carboxylatehydrochloride (245 mg, 0.5 mmol) and stirred for 72 hours at room temperature. NaCl (439 mg, 7.5 mmol) was then added to the reaction mixture, which was stirred for 1 hour and followed by the dropwise addition of acetone (300 mL) while stirring. The mixture was filtered. The filter cake was washed with acetone, and dried under vacuum to give the title compound as a white solid. (Sodium hyaluronate MW 50 KDa, 0.18 g, Yield: 44.2%, DS: 12%); ¹H NMR (400 MHz, D₂O) δ ppm 8.31-8.13 (m, 0.12H), 7.59-7.42 (m, 0.12H), 6.89-6.76 (m, 0.12H), 5.28-5.16 (m, 0.24H), 4.60-4.30 (m, 2H), 4.14-3.04 (m, 11.56H), 2.47-2.34 (d, J=31.1 Hz, 0.12H), 2.19-1.49 (m, 3.96H), 1.10-0.91 (m, 0.36H).

(Sodium hyaluronate MW 2000 KDa, 0.16 g, Yield: 39.3%, DS: 18%); ¹H NMR (400 MHz, D₂O) δ ppm 8.29-8.17 (m, 0.18H), 7.57-7.41 (m, 0.18H), 6.86-6.74 (m, 0.18H), 5.29-5.16 (m, 0.36H), 4.56-4.25 (m, 2H), 4.13-2.77 (m, 12.34H), 2.46-2.31 (d, J=31.1 Hz, 0.18H), 2.19-1.36 (m, 4.44H), 1.08-0.82 (m, 0.54H).

### Example 76

### Preparation of conjugate of 3-((3R,4R)-3-((7-((1S,4S)-2,5-diazabicyclo[2.2.1]heptane-2-carbonyl)-7H-pyrrolo[2,3-d]pyrimidin-4-yl)(methyl)amino)-4-methylpiperidin-1-yl)-3-oxopropanenitrile and HA

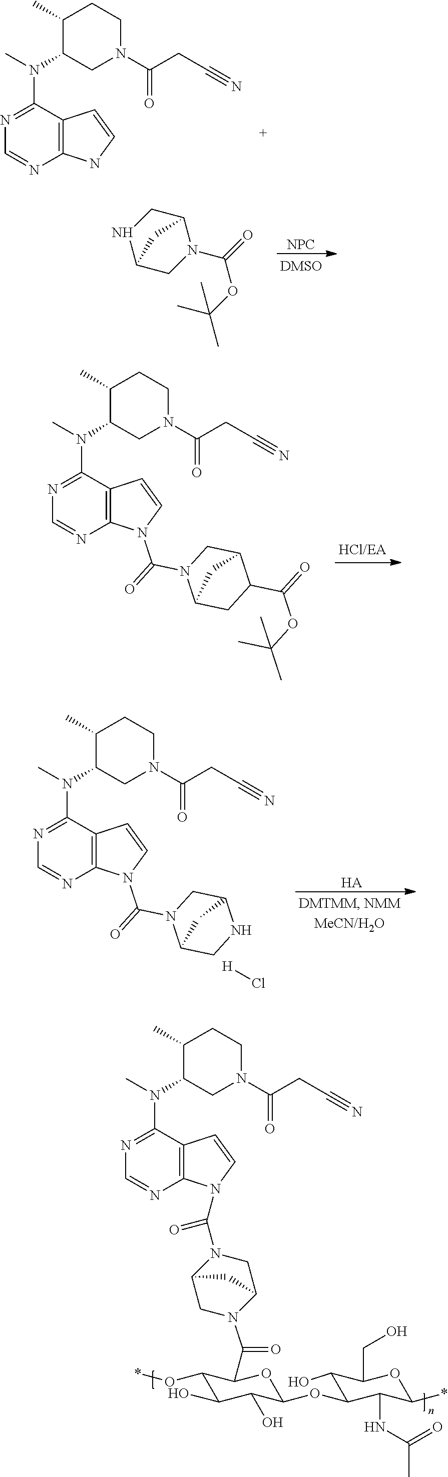

### Step 1: Preparation of tert-butyl (1S,4S)-5-(4-(((3R,4R)-1-(2-cyanoacetyl)-4-methylpiperidin-3-yl)(methyl)amino)-7H-pyrrolo[2,3-d]pyrimidine-7-carbonyl)-2,5-diazabicyclo[2.2.1]heptane-2-carboxylate

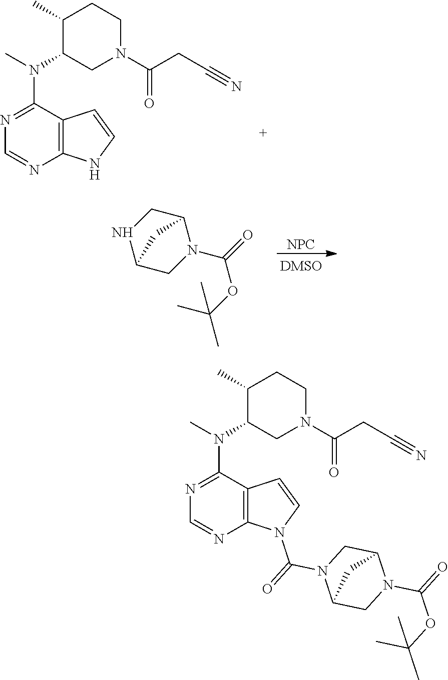

A mixture of 3-[(3R,4R)-4-methyl-3-[methyl(7H-pyrrolo[2,3-d]pyrimidin-4-yl)amino]-1-piperidyl]-3-oxo-propanenitrile (1250 mg, 4 mmol) and bis(4-nitrophenyl) carbonate (1460 mg, 4.8 mmol) in DMSO (25 mL) was stirred at room temperature for 7 hours. Then tert-butyl (1S,4S)-2,5-diazabicyclo[2.2.1]heptane-2-carboxylate (992 mg, 5 mmol) was added. The reaction mixture was stirred at 50° C. for 16 hours. The reaction mixture was poured into water and extracted with EtOAc. The organic layer was dried over sodium sulfate, filtered and then concentrated under reduced pressure. The crude residue was purified by column chromatography (DCM:Methanol=50:1) to afford the title compound (2 g, Yield: 93.1%). MS (m/z): [M+H]+ calcd for C₂₇H₃₆N₈O₄, 537.64; found: 537.2. ¹H NMR (400 MHz, Chloroform-d) δ 8.30 (d, J=5.9 Hz, 1H), 7.30 (d, J=9.4 Hz, 1H), 6.74-6.52 (m, 1H), 5.10 (s, 1H), 4.68 (d, J=124.9 Hz, 2H), 4.17-4.02 (m, 1H), 3.81 (d, J=53.0 Hz, 3H), 3.66-3.24 (m, 9H), 2.59-2.45 (m, 1H), 1.96 (qd, J=11.8, 9.6, 7.0 Hz, 2H), 1.77 (s, 2H), 1.46 (s, 9H), 1.11 (dd, J=10.0, 7.1 Hz, 3H).

### Step 2: Preparation of 3-((3R,4R)-3-((7-((1S,4S)-2,5-diazabicyclo[2.2.1]heptane-2-carbonyl)-7H-pyrrolo[2,3-d]pyrimidin-4-yl)(methyl)amino)-4-methylpiperidin-1-yl)-3-oxopropanenitrile hydrochloride

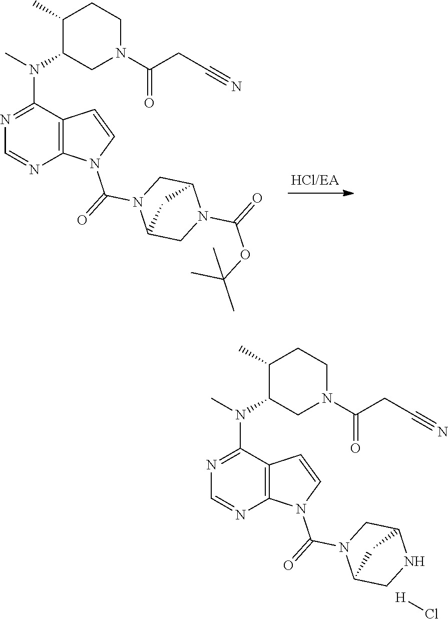

To a solution of tert-butyl (1S,4S)-5-(4-(((3R,4R)-1-(2-cyanoacetyl)-4-methylpiperidin-3-yl)(methyl)amino)-7H-pyrrolo[2,3-d]pyrimidine-7-carbonyl)-2,5-diazabicyclo[2.2.1]heptane-2-carboxylate (0.52 g, 0.97 mmol) in ethyl acetate (10 mL) was added 4M HCl in ethyl acetate solution (2 mL, 8 mmol) dropwise under N₂ at 0° C. The resulting reaction mixture was stirred at 0° C. for 30 min and then stirred at room temperature for 16 hours. The solution was diluted with ethyl acetate (20 mL) and concentrated under reduced pressure to afford the title compound as a white solid (0.45 g, Yield: 98.2%) that was used without further purification. MS (m/z): [M+H]+ calcd for C₂₂H₂₈N₈O₂, 437.52; found: 437.3.

### Step 3: Preparation of conjugate of 3-((3R,4R)-3-((7-((1S,4S)-2,5-diazabicyclo[2.2.1]heptane-2-carbonyl)-7H-pyrrolo[2,3-d]pyrimidin-4-yl)(methyl)amino)-4-methylpiperidin-1-yl)-3-oxopropanenitrile and HA

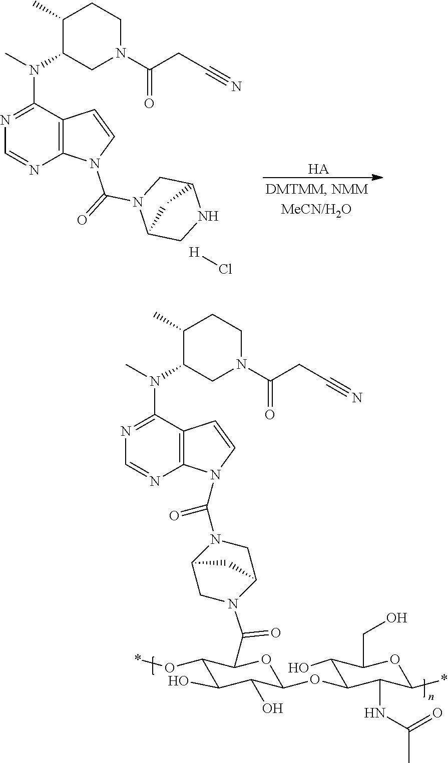

Sodium hyaluronate (161 mg, 0.4 mmol) was dissolved in 32 mL of deionized water in an 100 mL round-bottom flask followed by the dropwise addition of 21 mL of acetonitrile while stirring. To the solution was added 3-((3R,4R)-3-((7-((1S,4S)-2,5-diazabicyclo[2.2.1]heptane-2-carbonyl)-7H-pyrrolo[2,3-d]pyrimidin-4-yl)(methyl)amino)-4-methylpiperidin-1-yl)-3-oxopropanenitrile hydrochloride (132 mg, 0.28 mmol) and 4-methylmorpholine (NMM, 28 mg, 0.28 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 111 mg, 0.4 mmol) and stirred for 72 hours at room temperature. NaCl (234 mg, 4 mmol) was then added to the reaction mixture, which was stirred for 1 hour and followed by the dropwise addition of acetone (40 mL) while stirring. The mixture was filtered. The filter cake was collected, washed with acetone, and dried under vacuum to give the title compound as a white solid. (Sodium hyaluronate MW 2000 KDa, 0.19 g, Yield: 59.5%, DS: 29%); ¹H NMR (400 MHz, D₂O) δ 8.23-8.08 (m, 0.29H), 7.36-7.21 (m, 0.29H), 6.85-6.73 (m, 0.29H), 4.65-4.30 (m, 2.29H), 4.10-2.72 (m, 14.35H), 2.47-2.33 (m, 0.29H), 2.03-1.44 (m, 2.58H), 1.14-0.86 (m, 0.87H)

### Example 77

### Preparation of conjugate of 4-(((3R,4R)-1-(2-cyanoacetyl)-4-methylpiperidin-3-yl)(methyl)amino)-N-(4-(2-(methylamino)ethyl)phenyl)-7H-pyrrolo[2,3-d]pyrimidine-7-carboxamide and HA

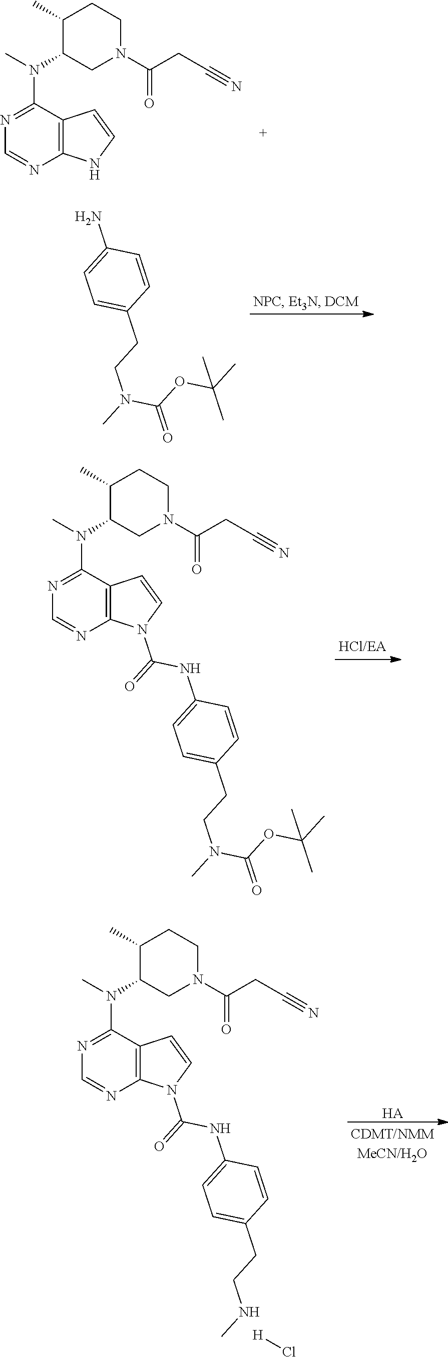

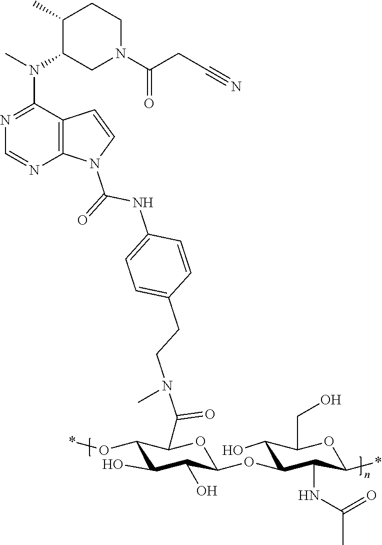

### Step 1: Preparation of tert-butyl (4-(4-(((3R,4R)-1-(2-cyanoacetyl)-4-methylpiperidin-3-yl)(methyl)amino)-7H-pyrrolo[2,3-d]pyrimidine-7-carboxamido)phenethyl)(methyl)carbamate

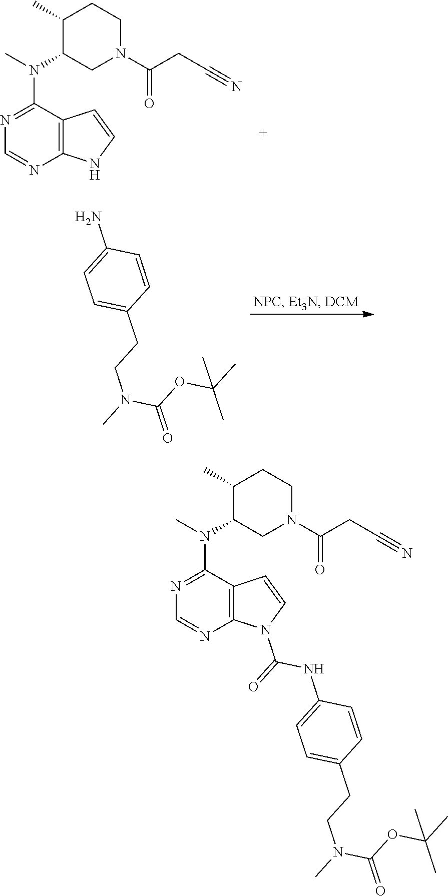

To a stirred mixture of 3-[(3R,4R)-4-methyl-3-[methyl(7H-pyrrolo[2,3-d]pyrimidin-4-yl)amino]-1-piperidyl]-3-oxo-propanenitrile (3442 mg, 11 mmol) and bis(4-nitrophenyl) carbonate (3352 mg, 11 mmol) in DCM (103 mL) was added triethylamine (2330 mg, 3.2 mL, 23 mmol). The reaction mixture was heated to 45° C. and stirred at this temperature for 6 hours. Then the reaction mixture was cooled to room temperature. Then tert-butyl (4-aminophenethyl)(methyl)carbamate (2300 mg, 9.19 mmol) was added. The reaction mixture was stirred at room temperature for 22 hours. The solution was diluted with DCM and washed with saturated NaHCO₃ solution, water and saturated brine solution. The organic layer was dried over sodium sulfate, filtered and then concentrated under reduced pressure. The crude residue was purified by column chromatography (EtOAc:Hexane=1:2-4:1) to afford the title compound (2.9 g, Yield: 53.7%). MS (m/z): [M+H]+ calcd for C₃₁H₄₀N₈O₄, 589.71; found: 589.2.

### Step 2: Preparation of 4-(((3R,4R)-1-(2-cyanoacetyl)-4-methylpiperidin-3-yl)(methyl)amino)-N-(4-(2-(methylamino)ethyl)phenyl)-7H-pyrrolo[2,3-d]pyrimidine-7-carboxamide hydrochloride

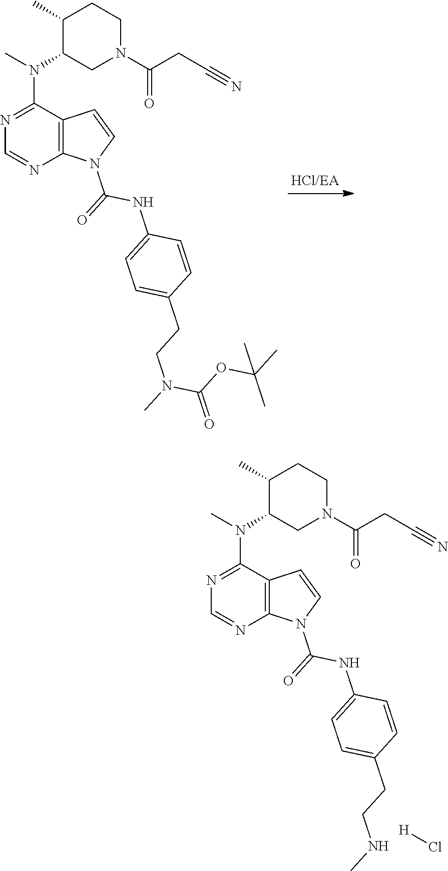

To a solution of tert-butyl (4-(4-(((3R,4R)-1-(2-cyanoacetyl)-4-methylpiperidin-3-yl)(methyl)amino)-7H-pyrrolo[2,3-d]pyrimidine-7-carboxamido)phenethyl)(methyl)carbamate (0.588 g, 1 mmol, 1 eq) in ethyl acetate (20 mL) was added 4M HCl in ethyl acetate solution (8 mL, 32 mmol) dropwise under N₂ at 0° C. The resulting reaction mixture was stirred at 0° C. for 30 min and then stirred at room temperature for 16 hours. The solution was diluted with ethyl acetate (20 mL) and concentrated under reduced pressure to afford the title compound as a white solid (0.48 g, Yield: 98.3%) that was used without further purification. MS (m/z): [M+H]+ calcd for C₂₆H₃₂N₈O₂, 489.60; found: 489.3.

### Step 3: Preparation of conjugate of 4-(((3R,4R)-1-(2-cyanoacetyl)-4-methylpiperidin-3-yl)(methyl)amino)-N-(4-(2-(methylamino)ethyl)phenyl)-7H-pyrrolo[2,3-d]pyrimidine-7-carboxamide and HA

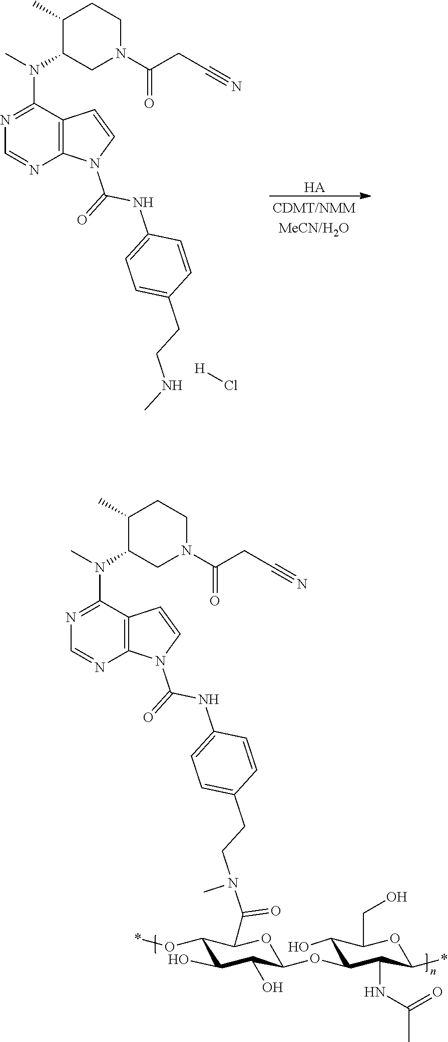

To a solution of Hyaluronic acid (121 mg, 0.3 mmol) in 24 mL of deionized water and 14 mL of acetonitrile was added 4-methylmorpholine (NMM, 21 mg, 0.21 mmol) and then cooled to 0° C. 2-Chloro-4,6-dimethoxy-1,3,5-triazine (83 mg, 0.3 mmol) was added and stirred at room temperature for 1 hour. 4-(((3R,4R)-1-(2-cyanoacetyl)-4-methylpiperidin-3-yl)(methyl)amino)-N-(4-(2-(methylamino)ethyl)phenyl)-7H-pyrrolo[2,3-d]pyrimidine-7-carboxamide hydrochloride (110 mg, 0.21 mmol) was added and stirred for 72 hours at room temperature. NaCl (175.5 mg, 3 mmol) was added to the reaction mixture and stirred for 1 hour. Acetone (300 mL) was added dropwisely while stirring and then the mixture was filtered. The filter cake was washed with acetone, and dried under vacuum to give the title compound as a white solid. (Sodium hyaluronate MW 50 KDa, 0.22 g, Yield: 86.3%, DS: 30%); ¹H NMR (400 MHz, D₂O) S 8.04-7.93 (m, 0.3H), 7.65-7.09 (m, 1.20H), 6.87-6.63 (m, 0.60H), 4.54-4.28 (m, 2.30H), 4.04-2.64 (m, 14.8H), 2.32-2.14 (m, 0.30H), 2.04-1.41 (m, 3.60H), 1.07-0.82 (m, 0.90H)

(Sodium hyaluronate MW 2000 KDa, 0.13 g, Yield: 51%, DS: 15%); ¹H NMR (400 MHz, D₂O) δ 8.02-7.87 (m, 0.15H), 7.34-6.88 (m, 0.60H), 6.79-6.36 (m, 0.30H), 4.55-4.14 (m, 2.15H), 4.07-2.51 (m, 12.4H), 2.36-2.22 (m, 0.15H), 1.87 (s, 3H), 1.47-1.37 (m, 0.30H), 1.05-0.80 (m, 0.45H)

### Example 78

### Preparation of conjugate of N-(3-(2-aminoethyl)phenyl)-4-(((3R,4R)-1-(2-cyanoacetyl)-4-methylpiperidin-3-yl)(methyl)amino)-7H-pyrrolo[2,3-d]pyrimidine-7-carboxamide and HA

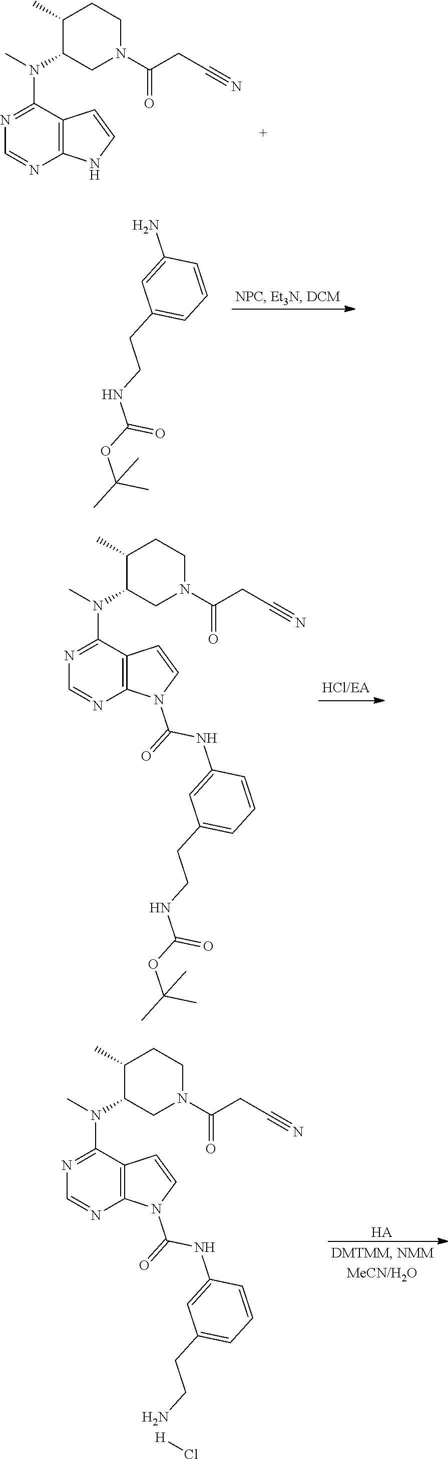

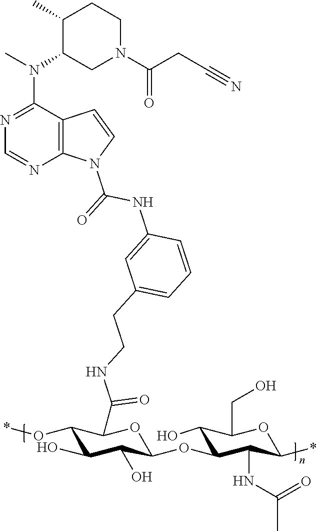

### Step 1: Preparation of tert-butyl (3-(4-(((3R,4R)-1-(2-cyanoacetyl)-4-methylpiperidin-3-yl)(methyl)amino)-7H-pyrrolo[2,3-d]pyrimidine-7-carboxamido)phenethyl)carbamate

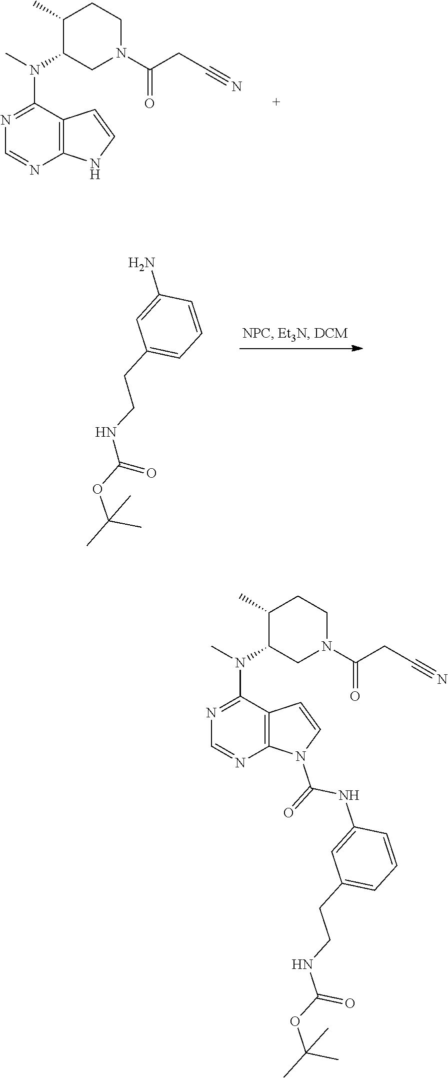 [SMILES: C[C@@H]1CCN(C(=O)CC#N)C[C@@H]1N(C)c1ncnc2[nH]ccc12 ; CC(C)(C)OC(=O)NCCc1cccc(N)c1 ; C[C@@H]1CCN(C(=O)CC#N)C[C@@H]1N(C)c1ncnc2c1ccn2C(=O)Nc1cccc(CCNC(=O)OC(C)(C)C)c1]

To a mixture of 3-[(3R,4R)-4-methyl-3-[methyl(7H-pyrrolo[2,3-d]pyrimidin-4-yl)amino]-1-piperidyl]-3-oxo-propanenitrile (1190 mg, 3.81 mmol) and bis(4-nitrophenyl) carbonate (1390 mg, 4.57 mmol) in DCM (24 mL) was added triethylamine (1.3 mL, 9.5 mmol). The reaction mixture was heated to 45° C. and stirred at this temperature for 6 hours. The reaction mixture was cooled to room temperature and tert-butyl (3-aminophenethyl)carbamate (900 mg, 3.81 mmol) was added. The reaction mixture was stirred at room temperature for 22 hours. The mixture was diluted with DCM and washed with saturated NaHCO₃ solution, water and saturated brine solution. The organic layer was dried over sodium sulfate, filtered and then concentrated under reduced pressure. The crude residue was purified by column chromatography (EtOAc:Hexane=1:2-4:5) to afford the title compound (0.687 g, Yield: 31.4%). MS (m/z): [M+H]+ calcd for C₃₀H₃₈N₈O₄, 575.69; found: 575.2.

### Step 2: Preparation of N-(3-(2-aminoethyl)phenyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride

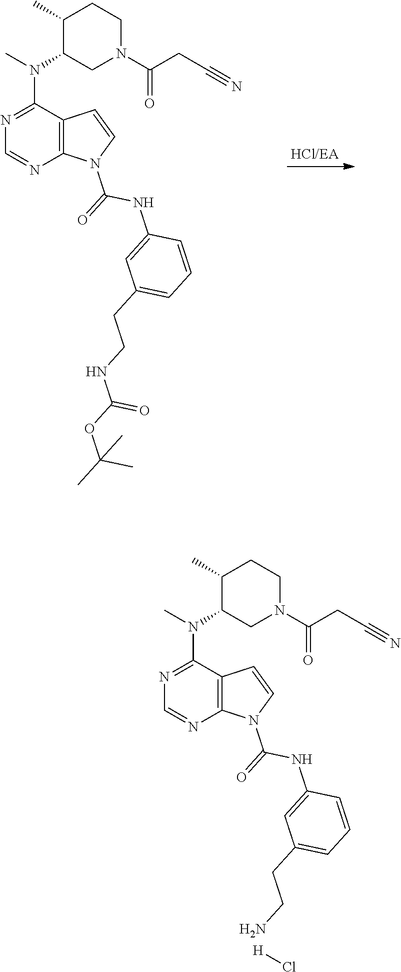

To a stirred solution of tert-butyl N-(3-(2-aminoethyl)phenyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride (0.66 g, 1.15 mmol) in EtOAc (13.2 mL) was slowly added 4M HCl in ethyl acetate (2.64 mL) at ice-bath. The reaction mixture was allowed to warm to room temperature and then was stirred at room temperature for 16 hours. The solution was diluted with EtOAc and concentrated under reduced pressure to afford the title compound as a white solid (0.55 g, Yield: 93.5%) that was used without further purification. MS (m/z): [M+H]+ calcd for C₂₅H₃₀N₈O₂, 475.57; found: 475.2.

### Step 3: Preparation of conjugate of N-(3-(2-aminoethyl)phenyl)-4-(((3R,4R)-1-(2-cyanoacetyl)-4-methylpiperidin-3-yl)(methyl)amino)-7H-pyrrolo[2,3-d]pyrimidine-7-carboxamide and HA

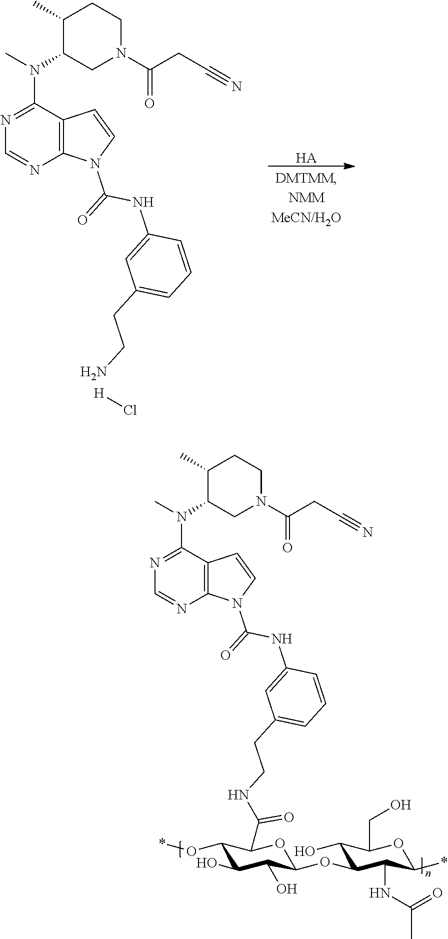

To a solution of Sodium hyaluronate (161 mg, 0.4 mmol, MW 50 KDa) in 32 mL of deionized water and 21 mL of acetonitrile were added N-(3-(2-aminoethyl)phenyl)-4-(((3R,4R)-1-(2-cyanoacetyl)-4-methylpiperidin-3-yl)(methyl)amino)-7H-pyrrolo[2,3-d]pyrimidine-7-carboxamide hydrochloride (143 mg, 0.28 mmol) and 4-methylmorpholine (NMM, 28 mg, 0.28 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 111 mg, 0.4 mmol) and stirred for 72 hours at room temperature. NaCl (234 mg, 4 mmol) was then added to the reaction mixture, which was stirred for 1 hour and followed by the dropwise addition of acetone (40 mL) while stirring. The mixture was filtered. The filter cake was washed with acetone, and dried under vacuum to give the title compound as a white solid. (Sodium hyaluronate MW 50 KDa, 0.234 g, Yield: 70%, DS: 50%); ¹H NMR (400 MHz, D₂O) δ 7.99-7.85 (m, 0.50H), 7.22-6.88 (m, 2H), 6.66-6.42 (m, 1H), 4.53-4.25 (m, 2.5H), 4.09-2.44 (m, 16.5H), 2.31-2.22 (m, 0.5H), 2.02-1.70 (m, 3H), 1.67-1.36 (m, 1H), 0.99-0.83 (m, 1.5H).

(Sodium hyaluronate MW 2000 KDa, 0.229 g, Yield: 68.5%, DSR: 40%); ¹H NMR (400 MHz, D₂O) δ 7.98-7.83 (m, 0.40H), 7.22-6.89 (m, 1.6H), 6.67-6.38 (m, 0.8H), 4.51-4.23 (m, 2.40H), 4.08-2.42 (m, 15.2H), 2.31-2.15 (m, 0.40H), 2.01-1.70 (m, 3H), 1.65-1.35 (m, 0.80H), 0.96-0.80 (m, 1.20H).

### Example 79

### Preparation of conjugate of N-(2-(2-aminoethoxy)pyridin-4-yl)-4-(((3R,4R)-1-(2-cyanoacetyl)-4-methylpiperidin-3-yl)(methyl)amino)-7H-pyrrolo[2,3-d]pyrimidine-7-carboxamide and HA

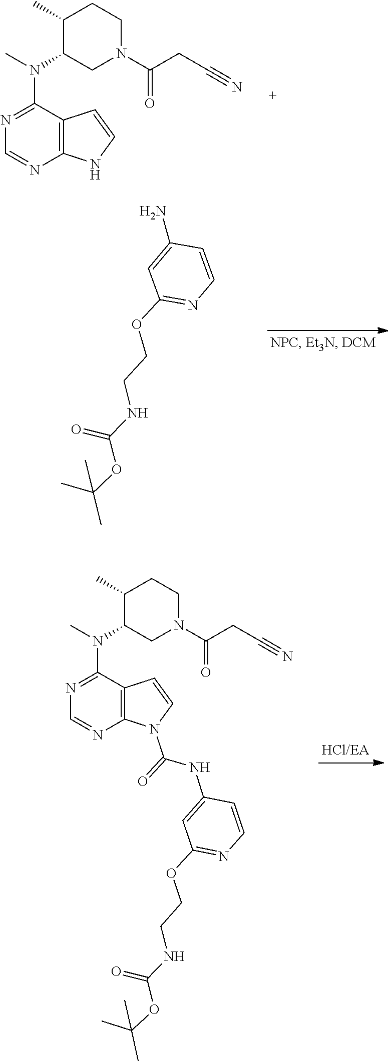 [SMILES: C[C@@H]1CCN(C(=O)CC#N)C[C@@H]1N(C)c1ncnc2[nH]ccc12 ; CC(C)(C)OC(=O)NCCOc1cc(N)ccn1 ; C[C@@H]1CCN(C(=O)CC#N)C[C@@H]1N(C)c1ncnc2c1ccn2C(=O)Nc1ccnc(OCCNC(=O)OC(C)(C)C)c1]

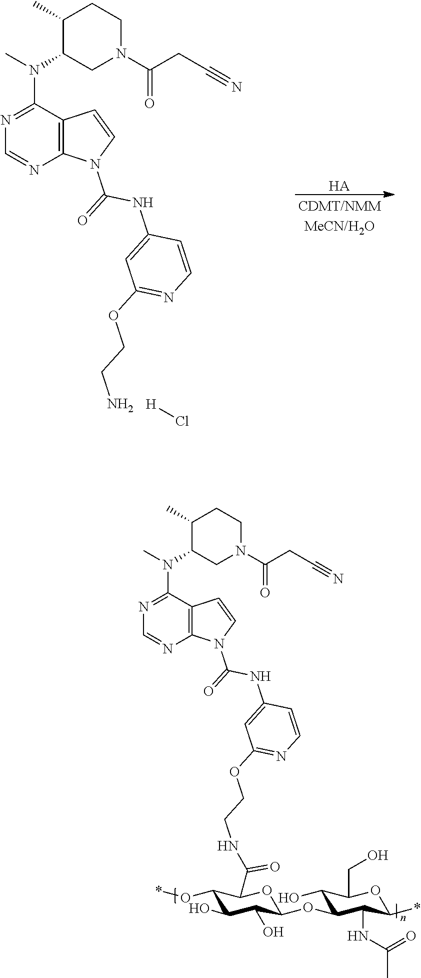

### Step 1: Preparation of tert-butyl (2-((4-(4-(((3R,4R)-1-(2-cyanoacetyl)-4-methylpiperidin-3-yl)(methyl)amino)-7H-pyrrolo[2,3-d]pyrimidine-7-carboxamido)pyridin-2-yl)oxy)ethyl)carbamate

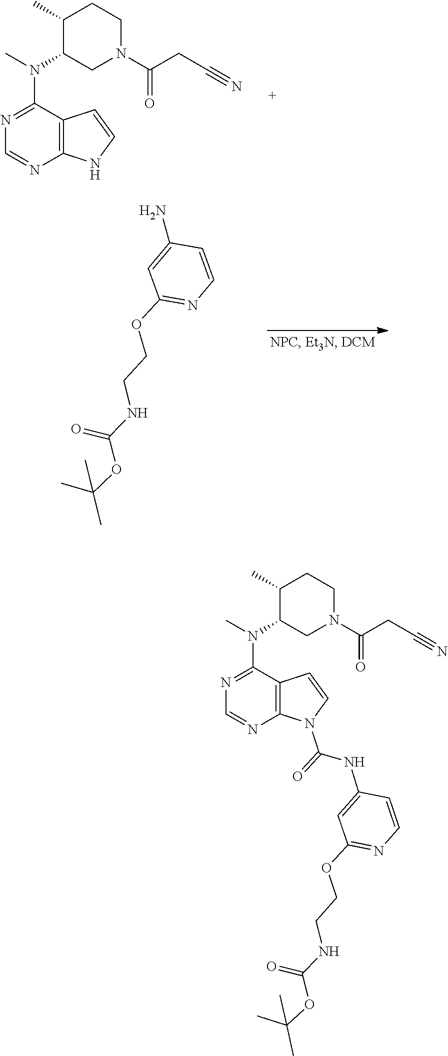 [SMILES: C[C@@H]1CCN(C(=O)CC#N)C[C@@H]1N(C)c1ncnc2[nH]ccc12 ; CC(C)(C)OC(=O)NCCOc1cc(N)ccn1 ; C[C@@H]1CCN(C(=O)CC#N)C[C@@H]1N(C)c1ncnc2c1ccn2C(=O)Nc1ccnc(OCCNC(=O)OC(C)(C)C)c1]

To a stirred mixture of 3-[(3R,4R)-4-methyl-3-[methyl(7H-pyrrolo[2,3-d]pyrimidin-4-yl)amino]-1-piperidyl]-3-oxo-propanenitrile (1190 mg, 3.81 mmol) and bis(4-nitrophenyl) carbonate (1390 mg, 4.57 mmol) in DCM (24 mL) was added triethylamine (1.3 mL, 9.5 mmol). The reaction mixture was heated to 45° C. and stirred at this temperature for 6 hours. Then the reaction mixture was cooled to room temperature. Then tert-butyl (2-((4-aminopyridin-2-yl)oxy)ethyl)carbamate (964 mg, 3.81 mmol) was added. The reaction mixture was stirred at room temperature for 22 hours. The solution was diluted with DCM and washed with saturated NaHCO₃ solution, water and saturated brine solution. The organic layer was dried over sodium sulfate, filtered and then concentrated under reduced pressure. The crude residue was purified by column chromatography (EtOAc:Hexane=1:2-4:5) to afford the title compound (0.45 g, Yield: 19.9%). MS (m/z): [M+H]+ calcd for C₂₉H₃₇N₉O₅, 592.67; found: 592.3.

### Step 2: Preparation of N-(2-(2-aminoethoxy)pyridin-4-yl)-4-(((3R,4R)-1-(2-cyanoacetyl)-4-methylpiperidin-3-yl)(methyl)amino)-7H-pyrrolo[2,3-d]pyrimidine-7-carboxamide hydrochloride

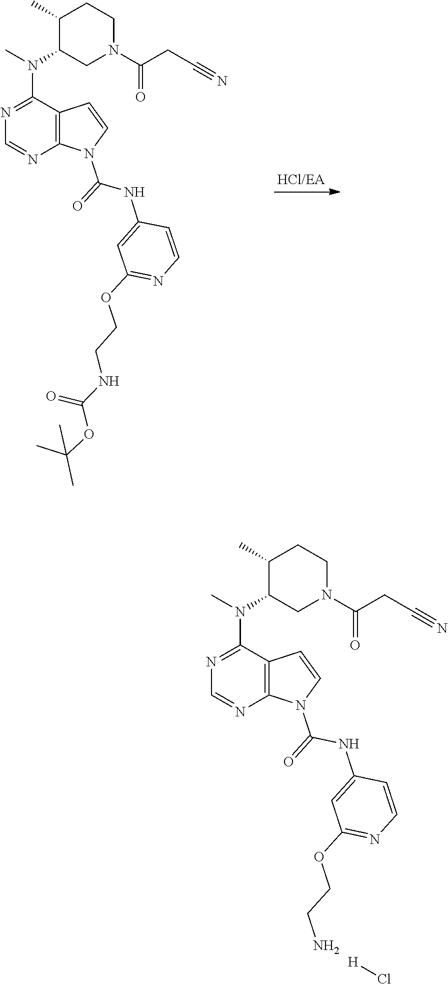

To a solution of tert-butyl (2-((4-(4-(((3R,4R)-1-(2-cyanoacetyl)-4-methylpiperidin-3-yl)(methyl)amino)-7H-pyrrolo[2,3-d]pyrimidine-7-carboxamido)pyridin-2-yl)oxy)ethyl)carbamate (450 mg, 0.76 mmol) in ethyl acetate (9 mL) was added 4M HCl in ethyl acetate solution (1.8 mL, 7.2 mmol) dropwise under N2 at 0° C. The resulting reaction mixture was stirred at 0° C. for 30 min and then stirred at room temperature for 20 hours. The solution was diluted with ethyl acetate (20 mL) and concentrated under reduced pressure to afford the title compound as a white solid (0.4 g, Yield: 99.7%) that was used without further purification. MS (m/z): [M+H]+ calcd for C₂₄H₂₉N₉O₃, 492.56; found: 492.3.

### Step 3: Preparation of conjugate of N-(2-(2-aminoethoxy)pyridin-4-yl)-4-(((3R,4R)-1-(2-cyanoacetyl)-4-methylpiperidin-3-yl)(methyl)amino)-7H-pyrrolo[2,3-d]pyrimidine-7-carboxamide and HA

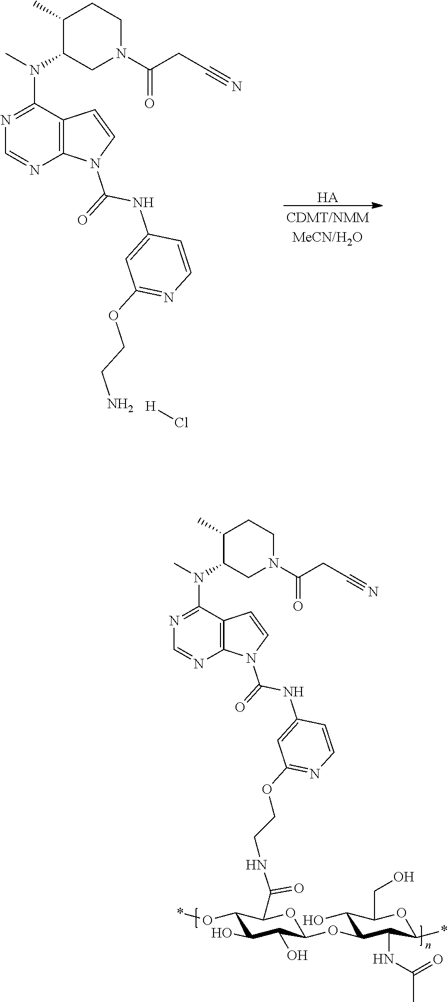

To a solution of sodium hyaluronate (0.153 g, 0.38 mmol, 1 eq) in Acetonitrile (22 mL) and H₂O (30 mL), 4-methylmorpholine (0.058 g, 0.38 mmol, 1.5 eq) and 2-chloro-4,6-dimethoxy-1,3,5-triazine (0.067 g, 0.38 mmol, 1 eq) were added at 0° C. The resulting reaction mixture was stirred at 0° C. for 30 min and then stirred at room temperature for 1 h. N-(2-(2-aminoethoxy)pyridin-4-yl)-4-(((3R,4R)-1-(2-cyanoacetyl)-4-methylpiperidin-3-yl)(methyl)amino)-7H-pyrrolo[2,3-d]pyrimidine-7-carboxamide hydrochloride (0.2 g, 0.38 mmol) was added to the reaction mixture and then the pH of the reaction mixture was adjusted to 6.5 to 7 with 4-methylmorpholine. The resulting reaction mixture was stirred 3 days at room temperature. NaCl (222 mg, 10 eq) in H₂O (2 mL) was added to the above reaction mixture and stirred for 0.5 b. Then acetone (350 mL) was added dropwise to the above mixture, while the precipitate was formed. The mixture was filtered. The filter cake was collected, washed with acetone, and dried under vacuo to give the title compound as a white solid. (Sodium hyaluronate MW 50 KDa, 0.167 g, Yield: 51.6%, DSR: 18%); ¹H NMR (400 MHz, D₂O) δ 8.52-8.19 (m, 0.18H), 7.87-7.34 (m, 0.54H), 7.08-6.68 (m, 0.36H), 4.63-4.31 (m, 2.18H), 4.13-2.93 (m, 12.34H), 2.50-2.29 (m, 0.18H), 2.22-1.52 (m, 3.36H), 1.14-0.86 (m, 0.54H).

(Sodium hyaluronate MW 2000 KDa, 0.137 g, Yield: 42.3%, DSR: 30%); ¹H NMR (400 MHz, D₂O) δ 8.42-8.28 (m, 0.30H), 7.86-7.45 (m, 0.90H), 7.12-6.77 (m, 0.60H), 4.59-4.29 (m, 2.30H), 4.13-2.82 (m, 13.90H), 2.47-2.29 (m, 0.30H), 2.03-1.47 (m, 3.60H), 1.06-0.85 (m, 0.90H).

### Example 80

### Preparation of conjugate of methyl N⁶-(4-(((3R,4R)-1-(2-cyanoacetyl)-4-methylpiperidin-3-yl)(methyl)amino)-7H-pyrrolo[2,3-d]pyrimidine-7-carbonyl)lysinate and HA

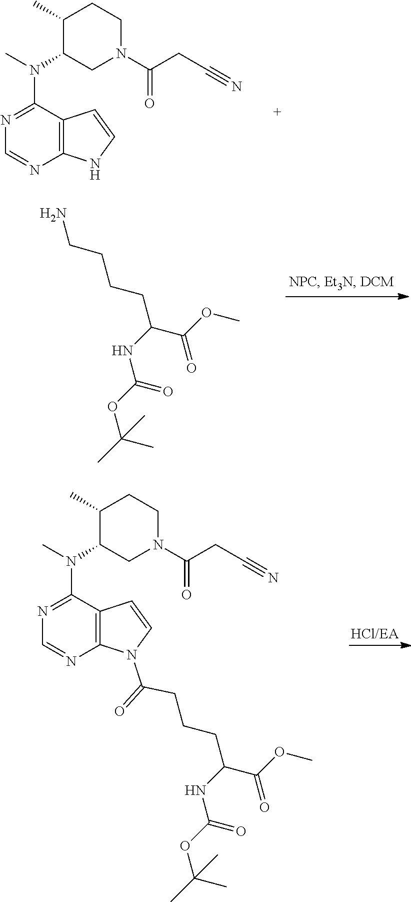

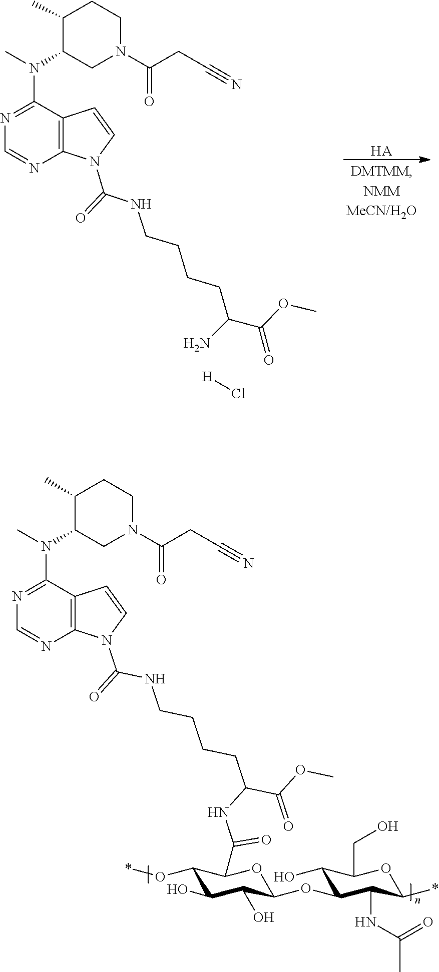

### Step 1: Preparation of methyl N²-(tert-butoxycarbonyl)-N⁶-(4-(((3R,4R)-1-(2-cyanoacetyl)-4-methylpiperidin-3-yl)(methyl)amino)-7H-pyrrolo[2,3-d]pyrimidine-7-carbonyl)lysinate

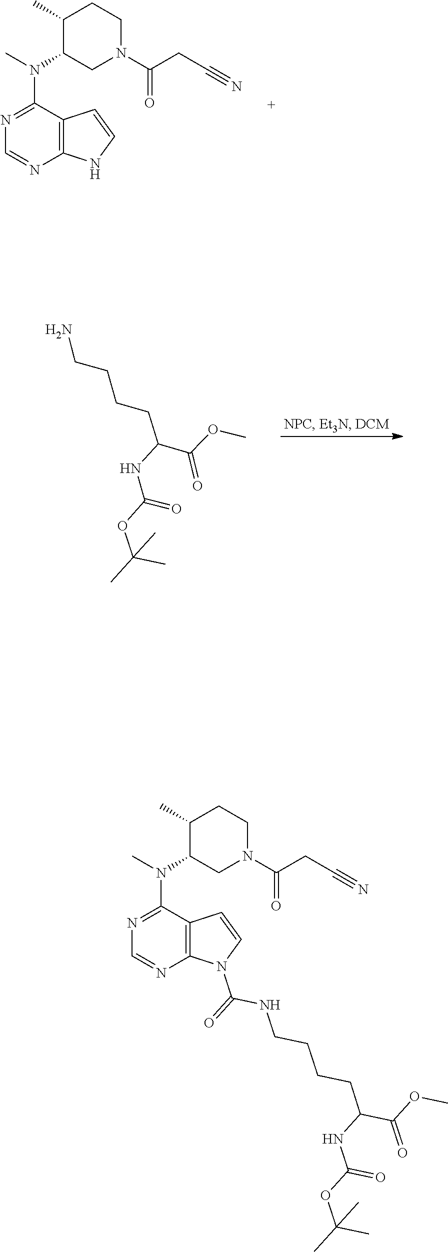

To a stirred mixture of 3-[(3R,4R)-4-methyl-3-[methyl(7H-pyrrolo[2,3-d]pyrimidin-4-yl)amino]-1-piperidyl]-3-oxo-propanenitrile (1561 mg, 5 mmol) and bis(4-nitrophenyl) carbonate (1824 mg, 6 mmol) in DCM (46 mL) was added triethylamine (1265 mg, 1.7 mL, 12.5 mmol). The reaction mixture was heated to 45° C. and stirred at this temperature for 16 hours. Then the reaction mixture was cooled to room temperature. Then methyl (tert-butoxycarbonyl)lysinate (1300 mg, 5 mmol) was added. The reaction mixture was stirred at room temperature for 22 hours. The solution was diluted with DCM and washed with saturated NaHCO₃ solution, water and saturated brine solution. The organic layer was dried over sodium sulfate, filtered and then concentrated under reduced pressure. The crude residue was purified by column chromatography (EtOAc:Hexane=1:5-3:1) to afford the title compound (0.6 g, Yield: 20%). MS (m/z): [M+H]+ calcd for C₂₉H₄₂N₈O₆, 599.71; found: 599.2.

### Step 2: Preparation of methyl N⁶-(4-(((3R,4R)-1-(2-cyanoacetyl)-4-methylpiperidin-3-yl)(methyl)amino)-7H-pyrrolo[2,3-d]pyrimidine-7-carbonyl)lysinate hydrochloride

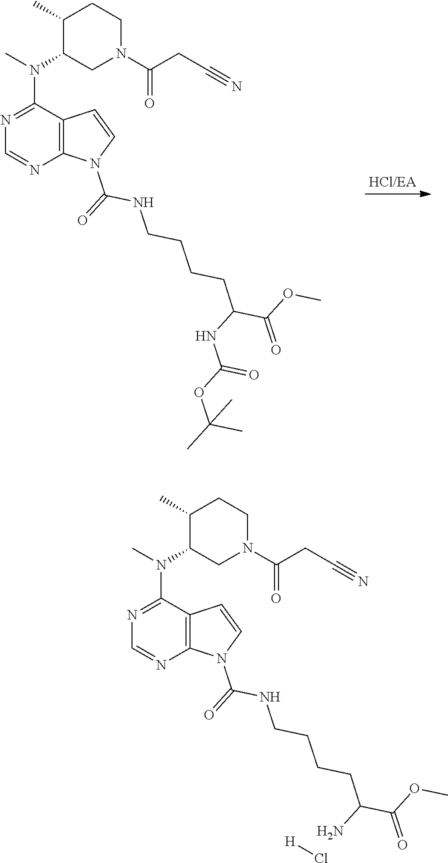

To a stirred solution of N²-(tert-butoxycarbonyl)-N6-(4-(((3R,4R)-1-(2-cyanoacetyl)-4-methylpiperidin-3-yl)(methyl)amino)-7H-pyrrolo[2,3-d]pyrimidine-7-carbonyl)lysinate (0.6 g, 1 mmol) in EtOAc (30 mL) was slowly added 4M HCl in ethyl acetate (2.7 mL) at ice-bath. The reaction mixture was allowed to warm to room temperature and then was stirred at room temperature for 16 hours. The solution was diluted with EtOAc and concentrated under reduced pressure to afford the title compound as a white solid (0.528 g, Yield: 98.8%) that was used without further purification. MS (m/z): [M+H]+ calcd for C₂₄H₃₄N₈O₄, 499.59; found: 499.2.

### Step 3: Preparation of conjugate of methyl N⁶-(4-(((3R,4R)-1-(2-cyanoacetyl)-4-methylpiperidin-3-yl)(methyl)amino)-7H-pyrrolo[2,3-d]pyrimidine-7-carbonyl)lysinate and HA

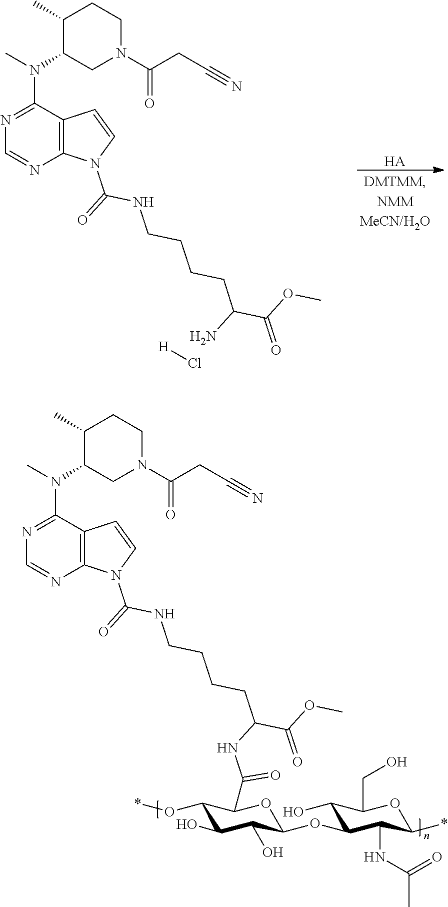

To a solution of Sodium hyaluronate (161 mg, 0.4 mmol,) in 32 mL and 21 mL of acetonitrile were added methyl N⁶-(4-(((3R,4R)-1-(2-cyanoacetyl)-4-methylpiperidin-3-yl)(methyl)amino)-7H-pyrrolo[2,3-d]pyrimidine-7-carbonyl)lysinate hydrochloride (150 mg, 0.28 mmol) and 4-methylmorpholine (NMM, 28 mg, 0.28 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 111 mg, 0.4 mmol) and stirred for 72 hours at room temperature. NaCl (234 mg, 4 mmol) was then added to the reaction mixture, which was stirred for 1 hour and followed by the dropwise addition of acetone (40 mL) while stirring. The mixture was filtered. The filter cake was washed with acetone, and dried under vacuum to give the title compound as a white solid.

(Sodium hyaluronate MW 50 KDa, 0.21 g, Yield: 61.1%, DS: 29%); ¹H NMR (400 MHz, D₂O) δ 8.00-7.43 (m, 0.29H), 7.29-6.64 (m, 0.29H), 6.44-5.78 (m, 0.29H), 4.63-4.22 (m, 2.29H), 4.12-2.67 (m, 14.35H), 2.44-1.29 (m, 4.32H), 1.12-0.81 (m, 0.87H).

(Sodium hyaluronate MW 2000 KDa, 0.19 g, Yield: 55.2%, DSR: 52%); ¹H NMR (400 MHz, D₂O) δ 8.00-7.53 (m, 0.52H), 7.31-6.67 (m, 0.52H), 6.43-5.87 (m, 0.52H), 4.62-4.18 (m, 2.52H), 4.10-2.52 (m, 17.80H), 2.41-1.29 (m, 7.16H), 1.11-0.78 (m, 1.56H).

### Example 81

### Preparation of conjugate of 3-((3R,4R)-3-((7-(6-amino-2-azaspiro[3.3]heptane-2-carbonyl)-7H-pyrrolo[2,3-d]pyrimidin-4-yl)(methyl)amino)-4-methylpiperidin-1-yl)-3-oxopropanenitrile and HA

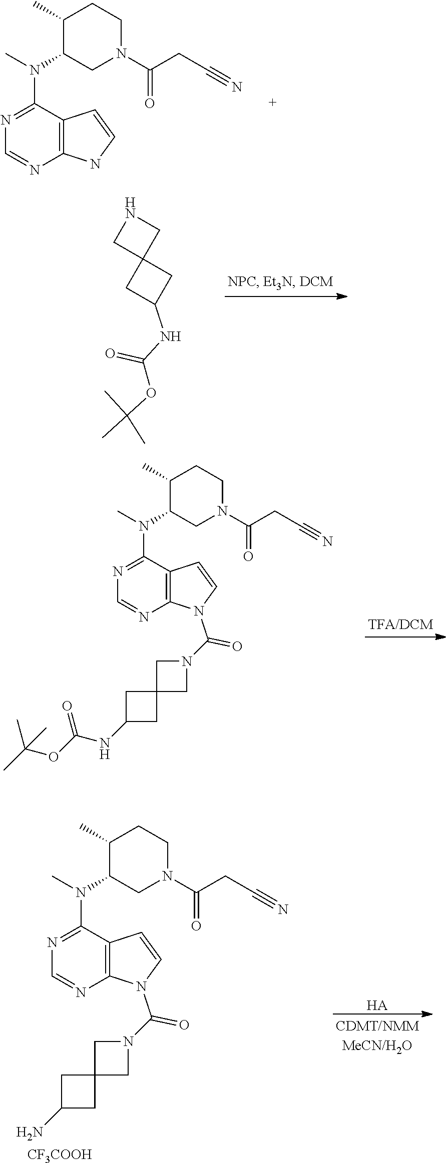

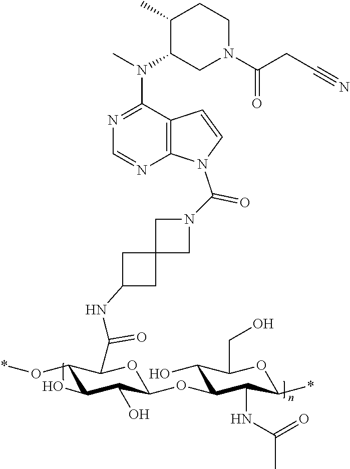

### Step 1: Preparation of tert-butyl (2-(4-(((3R,4R)-1-(2-cyanoacetyl)-4-methylpiperidin-3-yl)(methyl)amino)-7H-pyrrolo[2,3-d]pyrimidine-7-carbonyl)-2-azaspiro[3.3]heptan-6-yl)carbamate

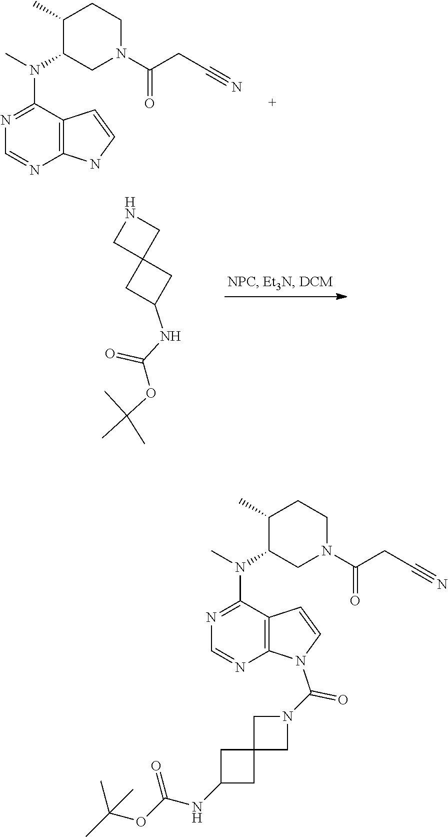 [SMILES: C[C@@H]1CCN(C(=O)CC#N)C[C@@H]1N(C)c1ncnc2[nH]ccc12 ; CC(C)(C)OC(=O)NC1CC2(CNC2)C1 ; C[C@@H]1CCN(C(=O)CC#N)C[C@@H]1N(C)c1ncnc2c1ccn2C(=O)N1CC2(CC(NC(=O)OC(C)(C)C)C2)C1]

To a stirred mixture of 3-[(3R,4R)-4-methyl-3-[methyl(7H-pyrrolo[2,3-d]pyrimidin-4-yl)amino]-1-piperidyl]-3-oxo-propanenitrile (1249.5 mg, 4 mmol) and bis(4-nitrophenyl) carbonate (1460 mg, 4.8 mmol) in DCM (120 mL) was added triethylamine (1012 mg, 1.39 mL, 10 mmol). The reaction mixture was heated to 45° C. and stirred at this temperature for 6 hours. Then the reaction mixture was cooled to room temperature. Then tert-butyl (2-azaspiro[3.3]heptan-6-yl)carbamate (938 mg, 4.4 mmol) was added. The reaction mixture was stirred at room temperature for 22 hours. The solution was diluted with DCM and washed with saturated NaHCO₃ solution, water and saturated brine solution. The organic layer was dried over sodium sulfate, filtered and then concentrated under reduced pressure. The crude residue was purified by column chromatography (EtOAc:Hexane=1:20-1:7) to afford the title compound (1.5 g; Yield: 68.1%). MS (m/z): [M+H]+ calcd for C₂₈H₃₈N₈O₄, 551.66; found: 551.2.

### Step 2: Preparation of 3-((3R,4R)-3-((7-(6-amino-2-azaspiro[3.3]heptane-2-carbonyl)-7H-pyrrolo[2,3-d]pyrimidin-4-yl)(methyl)amino)-4-methylpiperidin-1-yl)-3-oxopropanenitrile trifluoroacetic acid

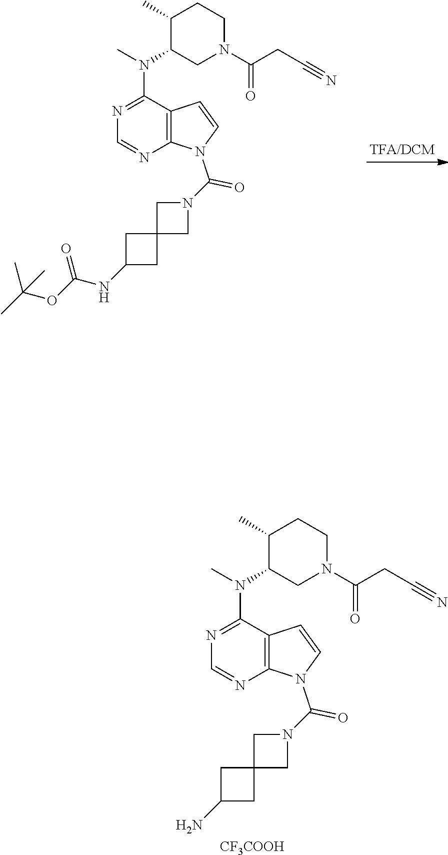 [SMILES: C[C@@H]1CCN(C(=O)CC#N)C[C@@H]1N(C)c1ncnc2c1ccn2C(=O)N1CC2(CC(NC(=O)OC(C)(C)C)C2)C1 ; "CF3COOH" ; C[C@@H]1CCN(C(=O)CC#N)C[C@@H]1N(C)c1ncnc2c1ccn2C(=O)N1CC2(CC(N)C2)C1]

To a stirred solution of tert-butyl (2-(4-(((3R,4R)-1-(2-cyanoacetyl)-4-methylpiperidin-3-yl)(methyl)amino)-7H-pyrrolo[2,3-d]pyrimidine-7-carbonyl)-2-azaspiro[3.3]heptan-6-yl)carbamate (275 mg, 0.5 mmol) in DCM (10 mL) was slowly added CF₃COOH (2 mL) at ice-bath. The reaction mixture was allowed to cool to room temperature and then was stirred at room temperature for 4 hours. The solution was diluted with DCM and concentrated under reduced pressure to afford the title compound as a brown oil (0.26 g, Yield: 78%) that was used without further purification. MS (m/z): [M+H]+ calcd for C₂₃H₃₀N₈O₂, 451.55; found: 451.3.

### Step 3: Preparation of conjugate of 3-((3R,4R)-3-((7-(6-amino-2-azaspiro[3.3]heptane-2-carbonyl)-7H-pyrrolo[2,3-d]pyrimidin-4-yl)(methyl)amino)-4-methylpiperidin-1-yl)-3-oxopropanenitrile and HA

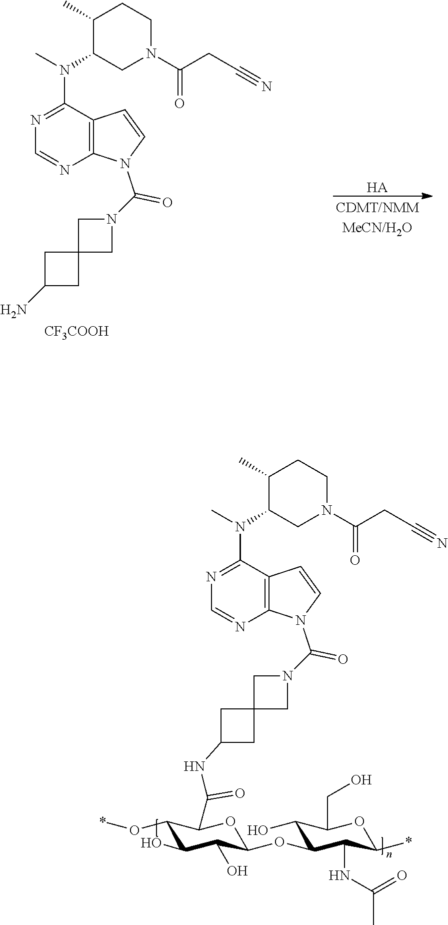

To a solution of Hyaluronic acid (201 mg, 0.5 mmol) in 40 mL of deionized water and ACN (17 ml) was added 4-methylmorpholine (NMM, 36 mg, 0.35 mmol). The solution was then cooled to 0° C., and 2-chloro-4,6-dimethoxy-1,3,5-triazine (201 mg, 0.5 mmol) was added and stirred at room temperature for 1 hour. The solution was mixed with 3-((3R,4R)-3-((7-(6-amino-2-azaspiro[3.3]heptane-2-carbonyl)-7H-pyrrolo[2,3-d]pyrimidin-4-yl)(methyl)amino)-4-methylpiperidin-1-yl)-3-oxopropanenitrile trifluoroacetic acid (197 mg, 0.35 mmol) and stirred for 72 hours at room temperature. NaCl (292.5 mg, 5 mmol) was then added to the reaction mixture, which was stirred for 1 hour and followed by the dropwise addition of acetone (300 mL) while stirring. The mixture was filtered. The filter cake was washed with acetone, and dried under vacuum to give the title compound as a white solid.

(Sodium hyaluronate, MW 50 KDa, 0.21 g, Yield: 51.7%, DS: 15%); ¹H NMR (400 MHz, Deuterium Oxide) δ 8.24-8.15 (m, 0.15H), 7.35-7.22 (m, 0.15H), 6.90-6.71 (m, 0.15H), 4.61-4.37 (m, 2.15H), 4.33-3.11 (m, 12.1H), 2.77-2.51 (m, 0.30H), 2.48-2.30 (m, 0.15H), 2.11-1.60 (m, 3.6H), 1.09-0.87 (m, 0.45H).

(Sodium hyaluronate MW 2000 KDa, 0.2 g, Yield: 49.3%, DSR: 11%); ¹H NMR (400 MHz, Deuterium Oxide) δ 8.26-7.99 (m, 0.11H), 7.36-7.17 (m, 0.11H), 6.88-5.73 (m, 0.11H), 4.62-4.35 (m, 2.11H), 4.34-3.04 (m, 11.54H), 2.73-2.52 (m, 0.22H), 2.46-2.29 (m, 0.11H), 2.12-1.54 (m, 3.44H), 1.11-0.81 (m, 0.33H).

### Example 82

### Preparation of conjugate of methyl (Z)-3-(((4-(N-methyl-2-(4-methylpiperazin-1-yl)acetamido)phenyl)amino)(phenyl)methylene)-2-oxo-1-(piperazine-1-carbonyl)indoline-6-carboxylate and HA

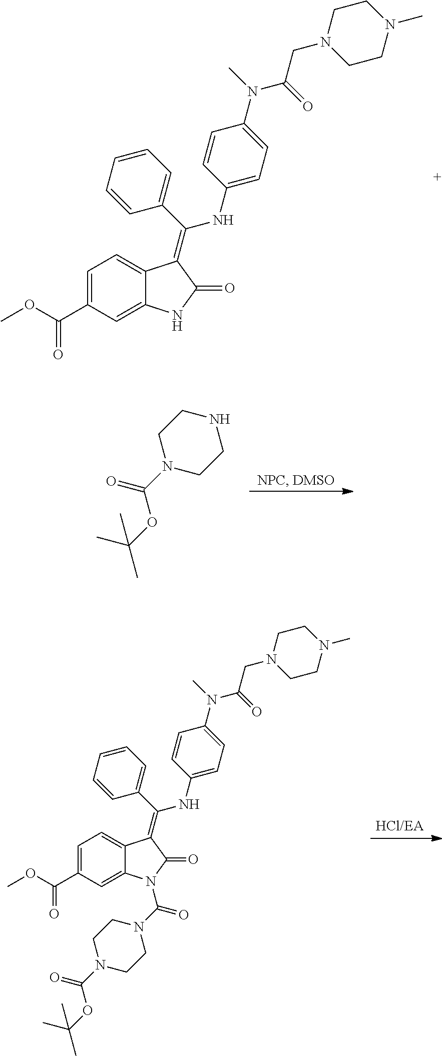

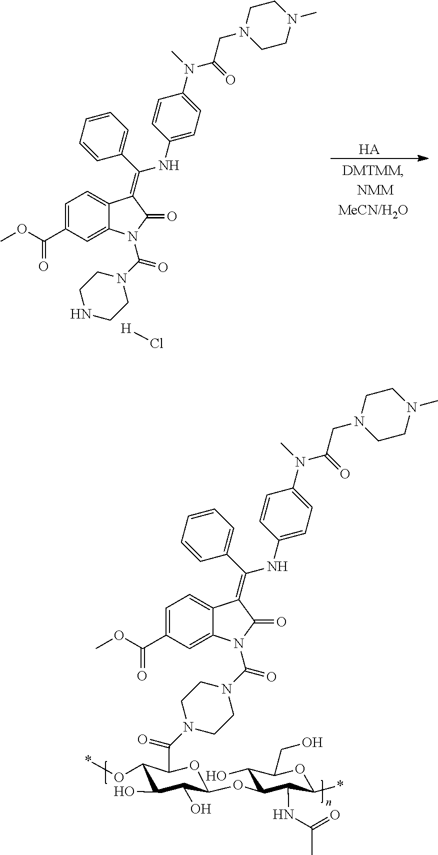

### Step 1: Preparation of methyl (Z)-1-(4-(tert-butoxycarbonyl)piperazine-1-carbonyl)-3-(((4-(N-methyl-2-(4-methylpiperazin-1-yl)acetamido)phenyl)amino)(phenyl)methylene)-2-oxoindoline-6-carboxylate

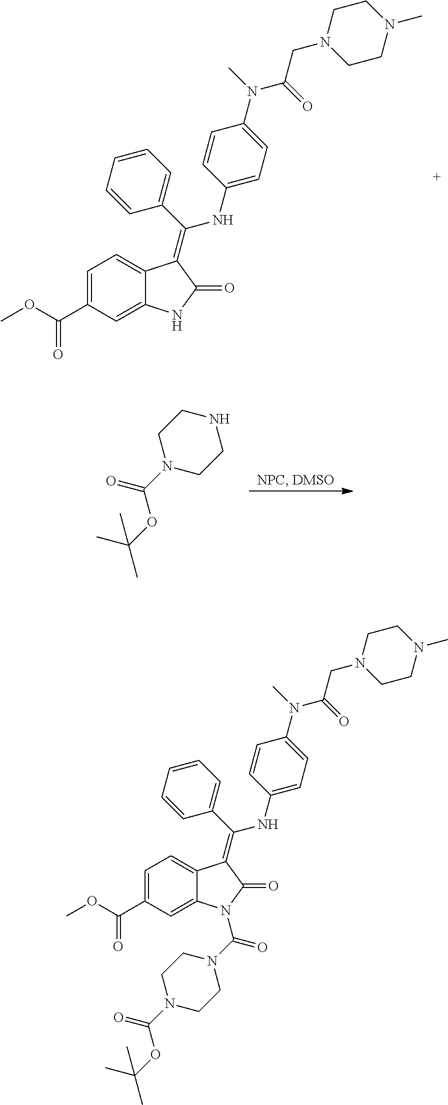

A mixture of methyl (3Z)-3-[[4-[methyl-[2-(4-methylpiperazin-1-yl)acetyl]amino]anilino]-phenyl-methylene]-2-oxo-indoline-6-carboxylate (1079 mg, 2 mmol) and bis(4-nitrophenyl) carbonate (668.8 mg, 2.2 mmol) in DMSO (30 mL) was stirred at room temperature for 7 hours. Then tert-Butyl hydrazinecarboxylate (264.3 mg, 2 mmol) was added. The reaction mixture was stirred at 50° C. for 16 hours. The reaction mixture was poured into water and extracted with EtOAc. The organic layer was dried over sodium sulfate, filtered and then concentrated under reduced pressure. The crude residue was purified by column chromatography (DCM:Methanol=8:1) to afford the title compound (0.5 g, Yield: 35.8%). MS (m/z): [M+H]+ calcd for C₄₁H₄₉N₇O₇, 752.89; found: 752.2. ¹H NMR (400 MHz, Chloroform-d) δ ppm 12.08 (s, 1H), 7.58 (dt, J=14.8, 7.2 Hz, 3H), 7.45 (dd, J=13.8, 6.9 Hz, 3H), 6.98 (d, J=8.2 Hz, 2H), 6.80 (d, J=8.2 Hz, 2H), 5.97 (d, J=8.3 Hz, 1H), 3.85 (s, 12H), 3.18 (s, 3H), 2.85 (d, J=36.9 Hz, 4H), 2.51 (d, J=21.2 Hz, 8H), 1.49 (s, 9H).

### Step 2: Preparation of methyl (Z)-3-(((4-(N-methyl-2-(4-methylpiperazin-1-yl)acetamido)phenyl)amino)(phenyl)methylene)-2-oxo-1-(piperazine-1-carbonyl)indoline-6-carboxylate hydrochloride

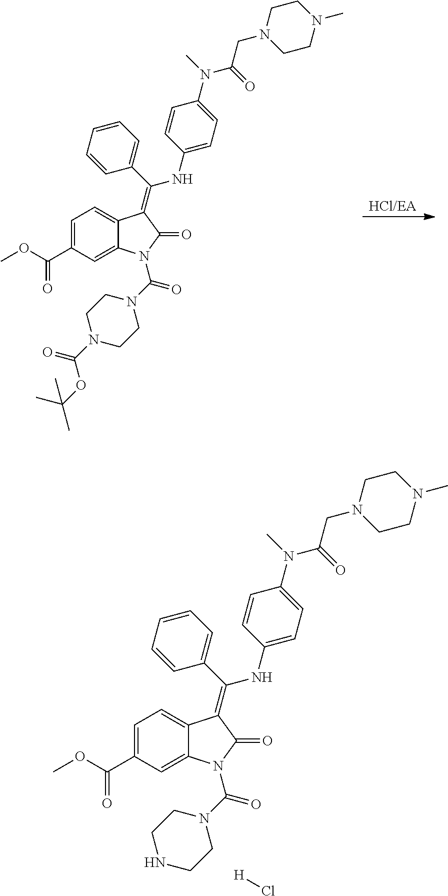

To a stirred solution of methyl (Z)-1-(4-(tert-butoxycarbonyl)piperazine-1-carbonyl)-3-(((4-(N-methyl-2-(4-methylpiperazin-1-yl)acetamido)phenyl)amino)(phenyl)methylene)-2-oxoindoline-6-carboxylate (0.4 g, 0.57 mmol) in EtOAc (4 mL) was slowly added 4M HCl in ethyl acetate (1.6 mL) at ice-bath. The reaction mixture was allowed to warm to room temperature and then was stirred at room temperature for 16 hours. The solution was diluted with EtOAc and concentrated under reduced pressure to afford the title compound as a white solid (0.36 g, Yield: 99%) that was used without further purification. MS (m/z): [M+H]+ calcd for C₃₆H₄₁N₇O₅, 652.77; found: 652.2.

### Step 3: Preparation of conjugate of methyl (Z)-3-(((4-(N-methyl-2-(4-methylpiperazin-1-yl)acetamido)phenyl)amino)(phenyl)methylene)-2-oxo-1-(piperazine-1-carbonyl)indoline-6-carboxylate and HA

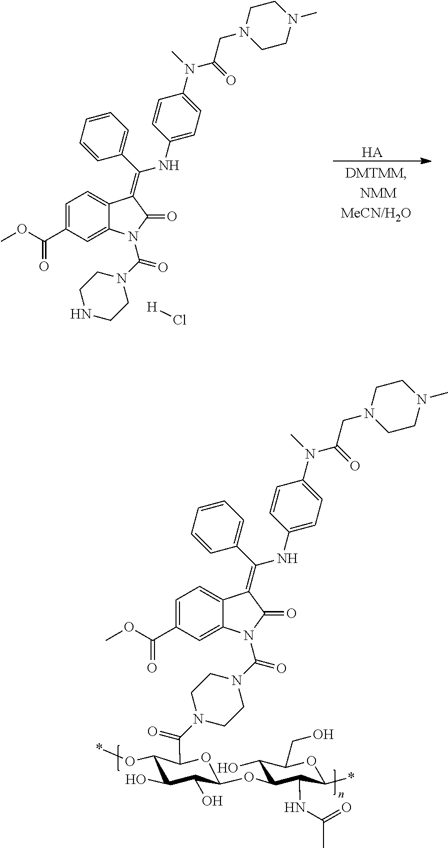

To a solution of Sodium hyaluronate (136 mg, 0.337 mmol) in 30 mL of deionized water and 19.5 mL of acetonitrile were added methyl (3Z)-1-(hydrazinecarbonyl)-3-[[4-[methyl-[2-(4-methylpiperazin-1-yl)acetyl]amino]anilino]-phenyl-methylene]-2-oxo-indoline-6-carboxylate hydrochloride (150 mg, 0.236 mmol) and 4-methylmorpholine (NMM, 23.9 mg, 0.236 mmol) at room temperature. The reaction mixture was stirred at this temperature for 2 hours. 4-(4,6-dimethoxy-1,3,5-triazin-2-yl)-4-methylmorpholinium chloride (DMTMM, 93 mg, 0.337 mmol) was added and stirred for 72 hours at room temperature. NaCl (197 mg, 3.37 mmol) was then added to the reaction mixture, which was stirred for 1 hour and followed by the dropwise addition of acetone (200 mL) while stirring. The mixture was filtered. The filter cake was washed with acetone, and dried under vacuum to give the title compound as a white solid. Sodium hyaluronate MW 50 KDa, 0.153 g, Yield: 44.8%, DS: 12%; ¹H NMR (400 MHz, Deuterium Oxide) δ 7.70-7.18 (m, 0.84H), 7.10-6.79 (m, 0.36H), 5.99-5.76 (m, 0.12H), 4.59-4.30 (m, 2H), 4.06-2.62 (m, 12.28H), 2.34-2.12 (m, 0.96H), 2.07-1.67 (m, 3H).

### Example 83

### Preparation of conjugate of methyl (Z)-1-((1S,4S)-2,5-diazabicyclo[2.2.1]heptane-2-carbonyl)-3-(((4-(N-methyl-2-(4-methylpiperazin-1-yl)acetamido)phenyl)amino)(phenyl)methylene)-2-oxoindoline-6-carboxylate and HA

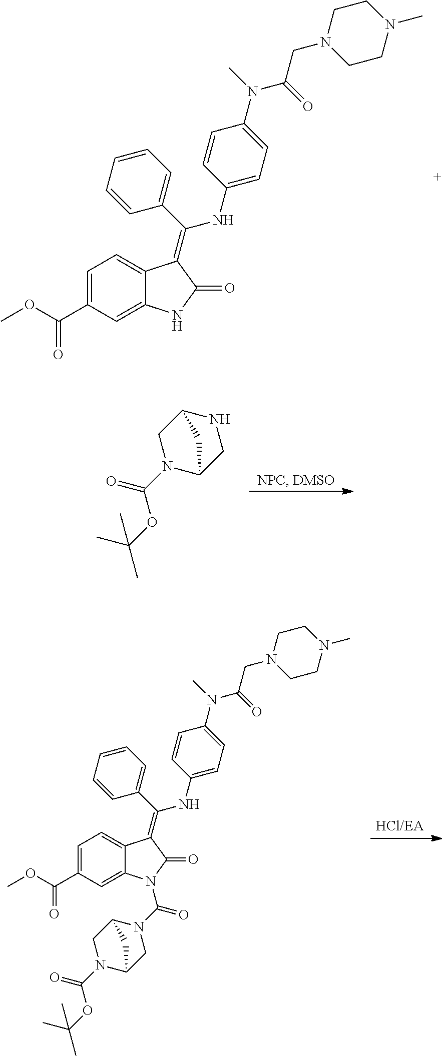

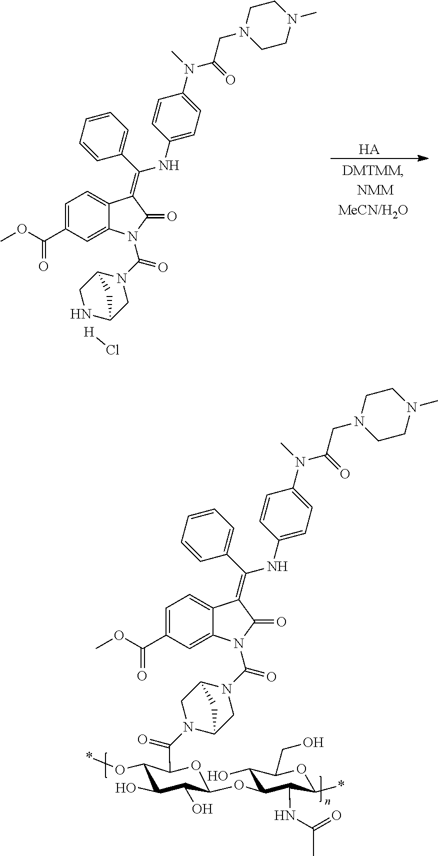

### Step 1: Preparation of methyl (Z)-1-((1S,4S)-5-(tert-butoxycarbonyl)-2,5-diazabicyclo[2.2.1]heptane-2-carbonyl)-3-(((4-(N-methyl-2-(4-methylpiperazin-1-yl)acetamido)phenyl)amino)(phenyl)methylene)-2-oxoindoline-6-carboxylate

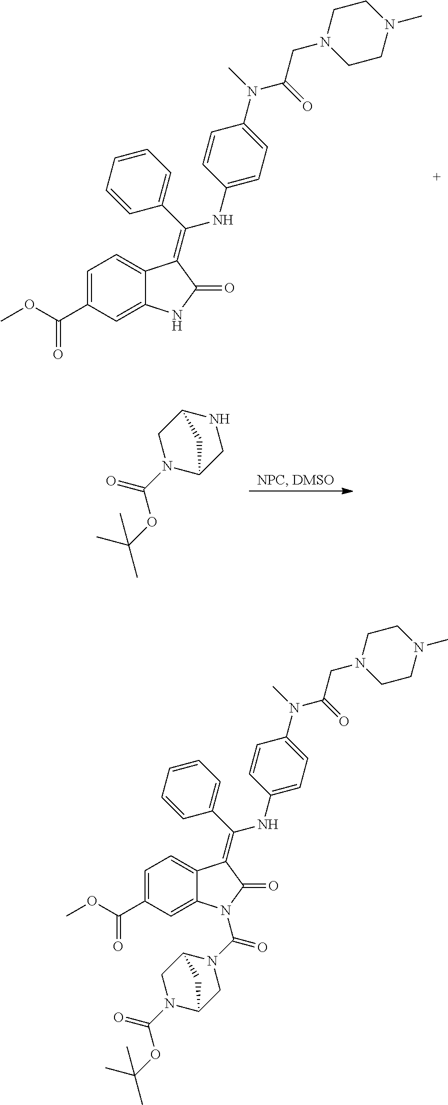

A mixture of methyl (3Z)-3-[[4-[methyl-[2-(4-methylpiperazin-1-yl)acetyl]amino]anilino]-phenyl-methylene]-2-oxo-indoline-6-carboxylate (1349 mg, 2.5 mmol) and bis(4-nitrophenyl) carbonate (912 mg, 3 mmol) in DMSO (27 mL) was stirred at room temperature for 7 hours. Then tert-butyl (1S,4S)-2,5-diazabicyclo[2.2.1]heptane-2-carboxylate (620 mg, 3.125 mmol) was added. The reaction mixture was stirred at 40° C. for 16 hours. The reaction mixture was poured into water and extracted with EtOAc. The organic layer was dried over sodium sulfate, filtered and then concentrated under reduced pressure. The crude residue was purified by column chromatography (DCM:Methanol=20:1) to afford the title compound (1.15 g, Yield: 60.3%). MS (m/z): [M+H]+ calcd for C₄₂H₄₉N₇O₇, 764.90; found: 764.2. ¹H NMR (400 MHz, Chloroform-d) δ 12.08 (s, 1H), 7.91 (d, J=15.4 Hz, 1H), 7.65-7.51 (m, 3H), 7.42 (dt, J=24.9, 7.1 Hz, 3H), 6.99 (dd, J=8.2, 4.9 Hz, 2H), 6.79 (t, J=7.8 Hz, 2H), 5.96 (dd, J=8.3, 5.2 Hz, 1H), 4.93 (s, 1H), 4.61 (s, 1H), 3.81 (d, J=38.8 Hz, 5H), 3.51 (s, 2H), 3.18 (s, 3H), 2.78 (s, 2H), 2.41 (s, 7H), 2.12-1.88 (m, 5H), 1.49 (m, 9H).

### Step 2: Preparation of methyl (Z)-1-((1S,4S)-2,5-diazabicyclo[2.2.1]heptane-2-carbonyl)-3-(((4-(N-methyl-2-(4-methylpiperazin-1-yl)acetamido)phenyl)amino)(phenyl)methylene)-2-oxoindoline-6-carboxylate hydrochloride

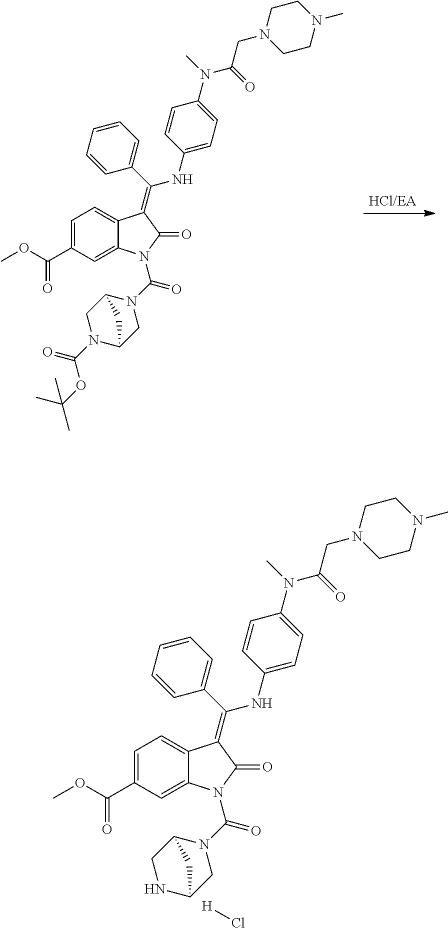

To a solution of methyl (Z)-1-((1S,4S)-5-(tert-butoxycarbonyl)-2,5-diazabicyclo[2.2.1]heptane-2-carbonyl)-3-(((4-(N-methyl-2-(4-methylpiperazin-1-yl)acetamido)phenyl)amino)(phenyl)methylene)-2-oxoindoline-6-carboxylate (600 mg, 0.786 mmol) in ethyl acetate (12 mL) was added 4M HCl in ethyl acetate solution (2.4 mL, 9.6 mmol) dropwise under N2 at 0° C. The resulting reaction mixture was stirred at 0° C. for 30 min and then stirred at room temperature for 20 hours. The solution was diluted with ethyl acetate (20 mL) and concentrated under reduced pressure to afford the title compound as a white solid (540 mg, Yield: 98%) that was used without further purification. MS (m/z): [M+H]+ calcd for C₃₇H₄₁N₇O₅, 664.32; found: 664.3.

### Step 3: Preparation of conjugate of methyl (Z)-1-((1S,4S)-2,5-diazabicyclo[2.2.1]heptane-2-carbonyl)-3-(((4-(N-methyl-2-(4-methylpiperazin-1-yl)acetamido)phenyl)amino)(phenyl)methylene)-2-oxoindoline-6-carboxylate and HA

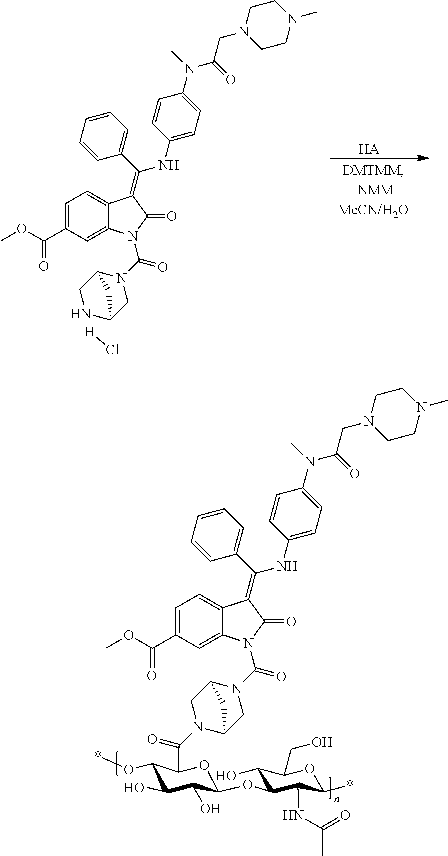

To a solution of Sodium hyaluronate (161 mg, 0.4 mmol) in 32 mL of deionized water and 21 mL of acetonitrile were added methyl (Z)-1-((1S,4S)-2,5-diazabicyclo[2.2.1]heptane-2-carbonyl)-3-(((4-(N-methyl-2-(4-methylpiperazin-1-yl)acetamido)phenyl)amino)(phenyl)methylene)-2-oxoindoline-6-carboxylatehydrochloride (56 mg, 0.08 mmol) and 4-methylmorpholine (NMM, 8 mg, 0.08 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 111 mg, 0.4 mmol) was added and stirred for 72 hours at room temperature. NaCl (234 mg, 4 mmol) was then added to the reaction mixture, which was stirred for 1 hour and followed by the dropwise addition of acetone (40 mL) while stirring. The mixture was filtered. The filter cake was collected, washed with acetone, and dried under vacuum to give the title compound as a white solid. (Sodium hyaluronate MW 50 KDa, 0.169 g, Yield: 41.2%, DSR: 14%); ¹H NMR (400 MHz, Deuterium Oxide) δ 7.69-7.22 (m, 0.84H), 7.21-6.78 (m, 0.56H), 5.93-5.82 (m, 0.14H), 4.96-4.79 (m, 0.14H), 4.50-4.16 (m, 2.14H), 4.00-2.49 (m, 12.66H), 2.30-2.16 (m, 0.7H), 2.00-1.52 (m, 3H).

### Example 84

### Preparation of conjugate of 4-(((3R,4R)-1-(2-cyanoacetyl)-4-methyl piperidin-3-yl)(methyl)amino)-7H-pyrrolo[2,3-d]pyrimidine-7-carbohydrazide and Chondroitin sulfate

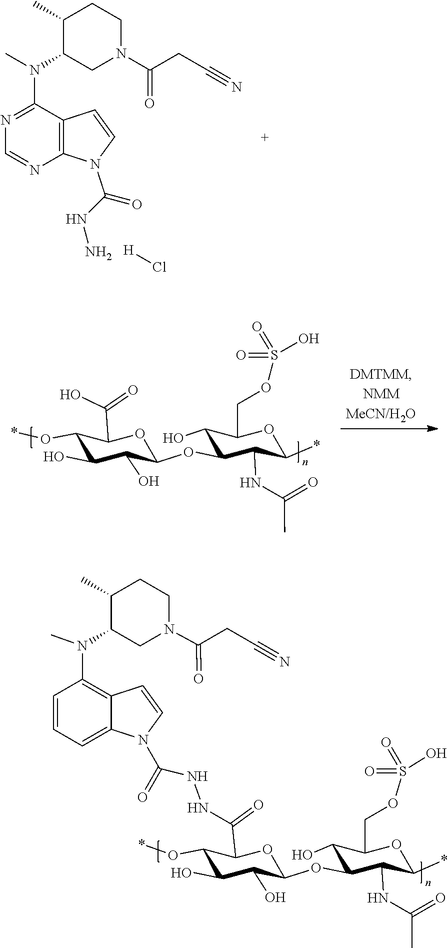

Chondroitin sulfate (231.5 mg, 0.5 mmol) was dissolved in 40 mL of deionized and 26 mL of acetonitrile. To the solution was added 4-(((3R,4R)-1-(2-cyanoacetyl)-4-methylpiperidin-3-yl)(methyl)amino)-7H-pyrrolo[2,3-d]pyrimidine-7-carbohydrazide (129.5 mg, 0.35 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 138 mg, 0.5 mmol) was added and stirred for 72 hours at room temperature. NaCl (293 mg, 5 mmol) was then added to the reaction mixture, which was stirred for 1 hour and followed by the dropwise addition of anhydrous alcohol (40 mL) while stirring. The mixture was filtered. The filter cake was washed with anhydrous alcohol, and dried under vacuum to give the title compound as a white solid (0.23 g, Yield: 55.5%, DSR: 19%). ¹H-NMR (400 MHz, D₂O): δ ppm 8.32-8.24 (m, 0.19H), 7.67-7.58 (m, 0.19H), 6.89-6.79 (m, 0.19H), 4.64-4.33 (m, 2.19H), 4.26-3.19 (m, 11.71H), 2.05-1.44 (m, 3.57H), 1.16-0.83 (m, 0.57H).

### Example 85

### Preparation of conjugate of 4-[1-[(1R)-2-cyano-1-cyclopentyl-ethyl]pyrazol-4-yl]pyrrolo[2,3-d]pyrimidine-7-carbohydrazide and Chondroitin sulfate

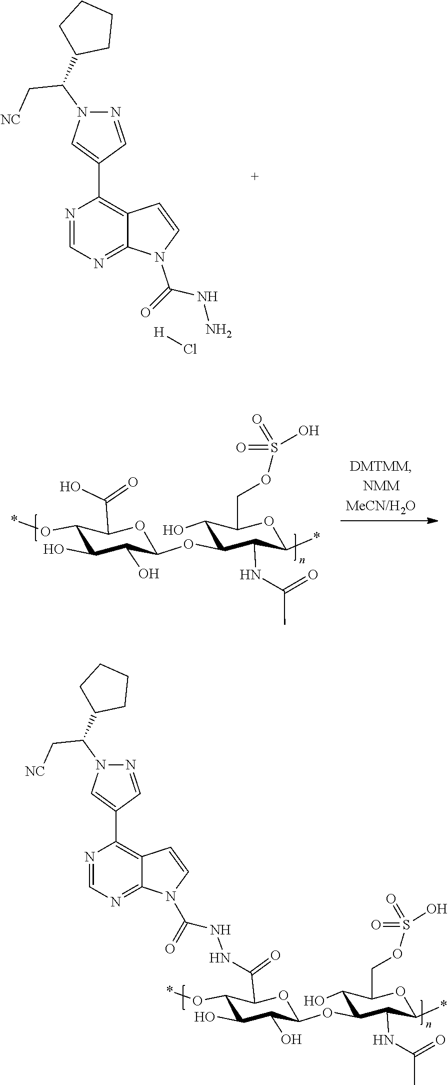

Chondroitin sulfate (266 mg, 0.58 mmol) was dissolved in 50 mL of deionized water and 25 mL of acetonitrile. To the solution was added 4-[1-[(1R)-2-cyano-1-cyclopentyl-ethyl]pyrazol-4-yl]pyrrolo[2,3-d]pyrimidine-7-carbohydrazide (230 mg, 0.58 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 189 mg, 0.68 mmol) and stirred for 72 hours at room temperature. NaCl (339 mg, 5.8 mmol) was then added to the reaction mixture, which was stirred for 1 hour and followed by the dropwise addition of anhydrous alcohol (40 mL) while stirring. The mixture was filtered. The filter cake was washed with anhydrous alcohol, and dried under vacuum to give the title compound as a white solid (0.135 g, Yield: 28.9%, DSR: 7%). ¹H-NMR (400 MHz, D₂O): δ ppm 8.94-8.57 (m, 0.14H), 8.44-8.34 (m, 0.07H), 8.03-7.88 (m, 0.07H), 7.39-7.29 (m, 0.07H), 4.67-4.34 (m, 2.07H), 4.33-3.04 (m, 10.14H), 2.23-1.78 (m, 3.14H), 1.77-1.26 (m, 0.56H).

### Example 86

### Preparation of conjugate of 4-[1-[3-(cyanomethyl)-1-ethylsulfonyl-azetidin-3-yl]pyrazol-4-yl]pyrrolo[2,3-d]pyrimidine-7-carbohydrazide and Chondroitin sulfate

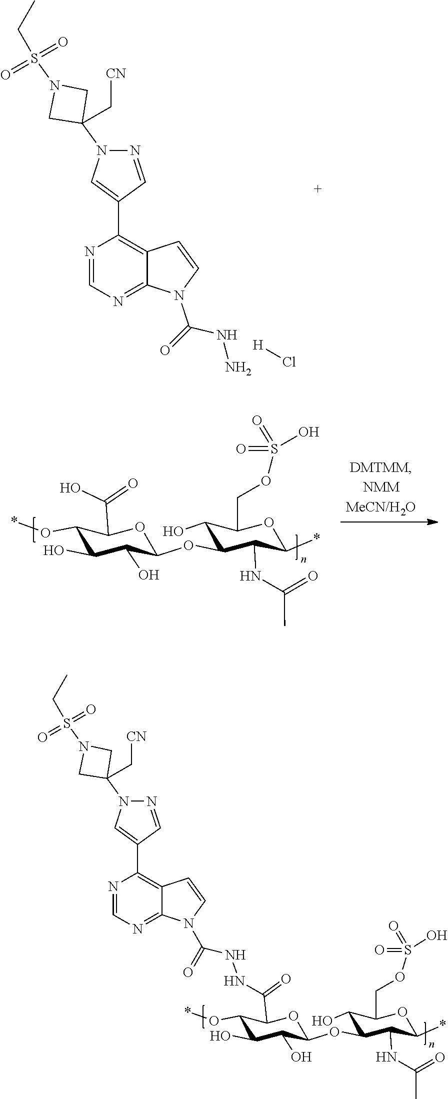

Chondroitin sulfate (200 mg, 0.43 mmol) was dissolved in 40 mL of deionized water and 20 mL of acetonitrile. To the solution was added 4-[1-[3-(cyanomethyl)-1-ethylsulfonyl-azetidin-3-yl]pyrazol-4-yl]pyrrolo[2,3-d]pyrimidine-7-carbohydrazide (200 mg, 0.43 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 141 mg, 0.51 mmol) and stirred for 72 hours at room temperature. NaCl (252 mg, 4.3 mmol) was then added to the reaction mixture, which was stirred for 1 hour and followed by the dropwise addition of anhydrous alcohol (40 mL) while stirring. The mixture was filtered. The filter cake was washed with anhydrous alcohol, and dried under vacuum to give the title compound as a white solid (0.177 g, Yield: 47.3%, DSR: 3%). ¹H-NMR (400 MHz, D₂O): δ ppm 9.06-8.86 (m, 0.06H), 8.50-8.42 (m, 0.03H), 8.14-8.06 (m, 0.03H), 7.51-7.31 (m, 0.03H), 4.62-4.28 (m, 2.12H), 4.24-3.05 (m, 10.12H), 2.55-1.57 (m, 3.09H).

### Example 87

### Preparation of conjugate of 1-[4-[[7-(hydrazinecarbonyl)pyrrolo[2,3-d]pyrimidin-4-yl]-methyl-amino]cyclohexyl]-N-methyl-methanesulfonamide and Chondroitin sulfate

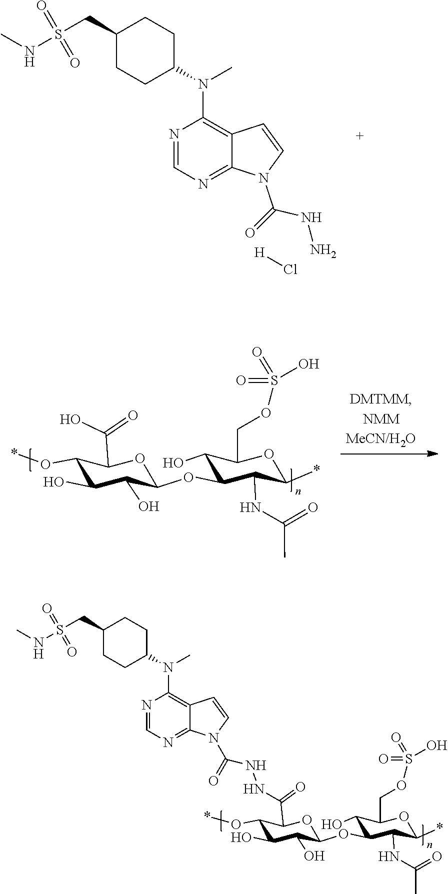

Chondroitin sulfate (250 mg, 0.54 mmol) was dissolved in 50 mL of deionized water and 25 mL of acetonitrile while stirring. To the solution was added 1-[4-[[7-(hydrazinecarbonyl)pyrrolo[2,3-d]pyrimidin-4-yl]-methyl-amino]cyclohexyl]-N-methyl-methanesulfonamide (230 mg, 0.54 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 175 mg, 0.54 mmol) and stirred for 72 hours at room temperature. NaCl (316 mg, 5.4 mmol) was then added to the reaction mixture, which was stirred for 1 hour and followed by the dropwise addition of anhydrous alcohol (40 mL) while stirring. The mixture was filtered. The filter cake was washed with anhydrous alcohol, and dried under vacuum to give the title compound as a white solid (0.314 g, Yield: 69.5%, DSR: 16%). ¹H-NMR (400 MHz, D₂O): δ ppm 8.44-8.35 (m, 0.16H), 7.91-7.80 (m, 0.16H), 7.04-6.92 (m, 0.16H), 4.64-4.36 (m, 2H), 4.35-3.05 (m, 10.48H), 3.03-2.92 (m, 0.32H), 2.77-2.60 (m, 0.48H), 2.56-2.39 (m, 0.16H), 2.20-1.58 (m, 3.8H), 1.49-1.30 (m, 0.64H).

### Example 88

### Preparation of conjugate of methyl (4-(((3R,4R)-1-(2-cyanoacetyl)-4-methylpiperidin-3-yl)(methyl)amino)-7H-pyrrolo[2,3-d]pyrimidine-7-carbonyl)-L-lysinate and Chondroitin sulfate

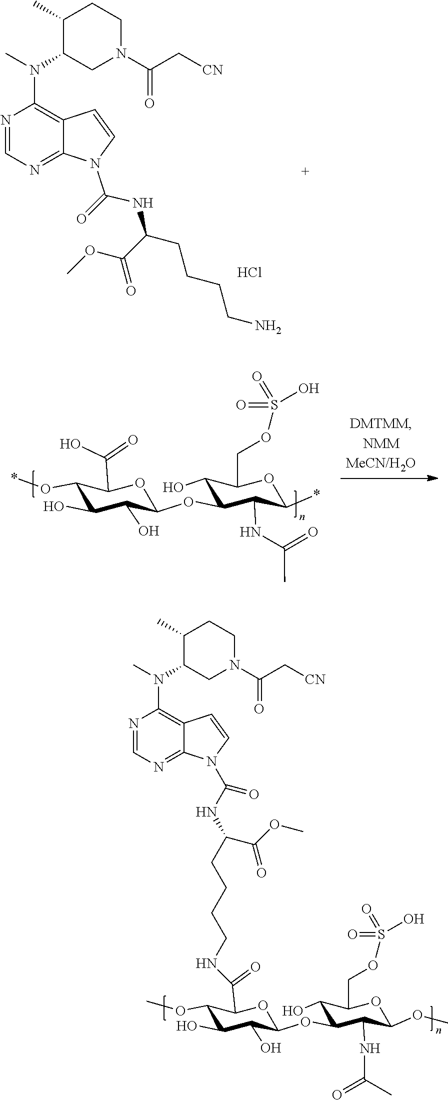

Chondroitin sulfate (231.5 mg, 0.5 mmol) was dissolved in 46 mL of deionized water and 23 mL of acetonitrile while stirring. To the solution was added methyl (4-(((3R,4R)-1-(2-cyanoacetyl)-4-methylpiperidin-3-yl)(methyl)amino)-7H-pyrrolo[2,3-d]pyrimidine-7-carbonyl)-L-lysinate hydrochloride (187 mg, 0.35 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 145 mg, 0.53 mmol) was added and stirred for 72 hours at room temperature. NaCl (293 mg, 5 mmol) was then added to the reaction mixture, which was stirred for 1 hour and followed by the dropwise addition of anhydrous alcohol (40 mL) while stirring. The mixture was filtered. The filter cake was washed with anhydrous alcohol, and dried under vacuum to give the title compound as a white solid (0.23 g, Yield: 48.1%, DSR: 35%). ¹H NMR (400 MHz, D₂O) δ 8.20-7.68 (m, 0.35H), 7.33-6.95 (m, 0.35H), 6.47-6.02 (m, 0.35H), 4.80-4.18 (m, 2.35H), 4.11-2.78 (m, 15.25H), 2.33-1.16 (m, 5.8H), 1.11-0.78 (m, 1.05H).

### Example 89

### Preparation of conjugate of methyl (2S)-6-amino-2-[[4-[1-[(1R)-2-cyano-1-cyclopentyl-ethyl]pyrazol-4-yl]pyrrolo[2,3-d]pyrimidine-7-carbonyl]amino]hexanoate and Chondroitin sulfate

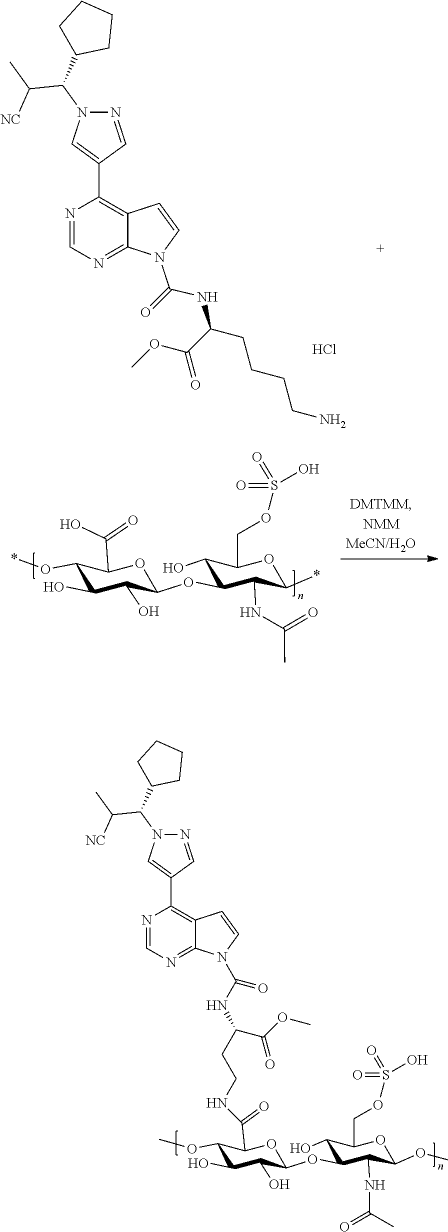

Chondroitin sulfate (184 mg, 0.4 mmol) was dissolved in 42 mL of deionized water and 21 mL of acetonitrile while stirring. To the solution was added methyl (2S)-6-amino-2-[[4-[1-[(1R)-2-cyano-1-cyclopentyl-ethyl]pyrazol-4-yl]pyrrolo[2,3-d]pyrimidine-7-carbonyl]amino]hexanoate (210 mg, 0.4 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 130 mg, 0.47 mmol) and stirred for 72 hours at room temperature. NaCl (234 mg, 4 mmol) was then added to the reaction mixture, which was stirred for 1 hour and followed by the dropwise addition of anhydrous alcohol (40 mL) while stirring. The mixture was filtered. The filter cake was washed with anhydrous alcohol, and dried under vacuum to give the title compound as a white solid. (0.153 g, Yield: 39%, DS: 5%) H-NMR (400 MHz, D₂O): δ ppm 8.96-8.8 (m, 0.1H), 8.44-8.34 (m, 0.05H), 8.05-7.92 (m, 0.05H), 7.40-7.27 (m, 0.05H), 4.62-4.36 (m, 2.1H), 4.29-3.03 (m, 10.25H), 3.02-2.9 (m, 0.1H), 2.47-2.25 (m, 0.05H), 2.20-1.81 (m, 3.15H), 1.72-1.24 (m, 0.55H).

### Example 90

### Preparation of conjugate of methyl (2S)-6-amino-2-[[4-[1-[3-(cyanomethyl)-1-ethylsulfonyl-azetidin-3-yl]pyrazol-4-yl]pyrrolo[2,3-d]pyrimidine-7-carbonyl]amino]hexanoate and Chondroitin sulfate

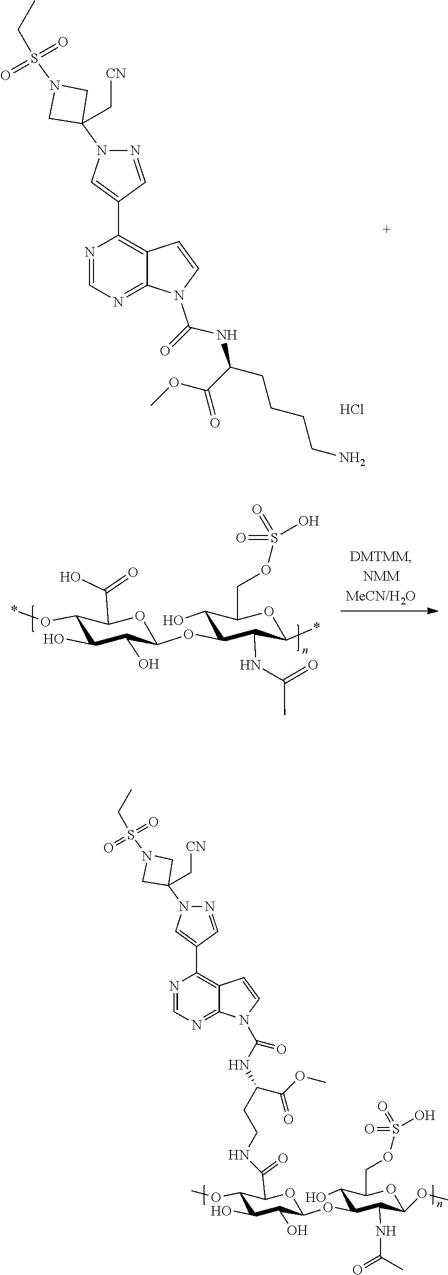

Chondroitin sulfate (117 mg, 0.25 mmol) was dissolved in 35 mL of deionized water and 20 mL of acetonitrile while stirring. To the solution was added methyl (2S)-6-amino-2-[[4-[1-[3-(cyanomethyl)-1-ethylsulfonyl-azetidin-3-yl]pyrazol-4-yl]pyrrolo[2,3-d]pyrimidine-7-carbonyl]amino]hexanoate (150 mg, 0.25 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 83 mg, 0.3 mmol) and stirred for 72 hours at room temperature. NaCl (146 mg, 2.5 mmol) was then added to the reaction mixture, which was stirred for 1 hour and followed by the dropwise addition of anhydrous alcohol (40 mL) while stirring. The mixture was filtered. The filter cake was washed with anhydrous alcohol, and dried under vacuum to give the title compound as a white solid (0.123 g, Yield: %, DS: 4%). ¹H-NMR (400 MHz, D₂O): δ ppm 9.04-8.96 (m, 0.08H), 8.49-8.41 (m, 0.04H), 8.12-8.03 (m, 0.04H), 7.49-7.33 (m, 0.04H), 4.62-4.29 (m, 2.16H), 4.25-3.05 (m, 10.32H), 3.03-2.84 (m, 0.08H), 2.55-1.50 (m, 3.08H), 1.47-1.19 (m, 0.28H).

### Example 91

### Preparation of conjugate of methyl (2S)-6-amino-2-[[4-[methyl-[4-(methylsulfamoylmethyl)cyclohexyl]amino]pyrrolo[2,3-d]pyrimidine-7-carbonyl]amino]hexanoate and Chondroitin sulfate

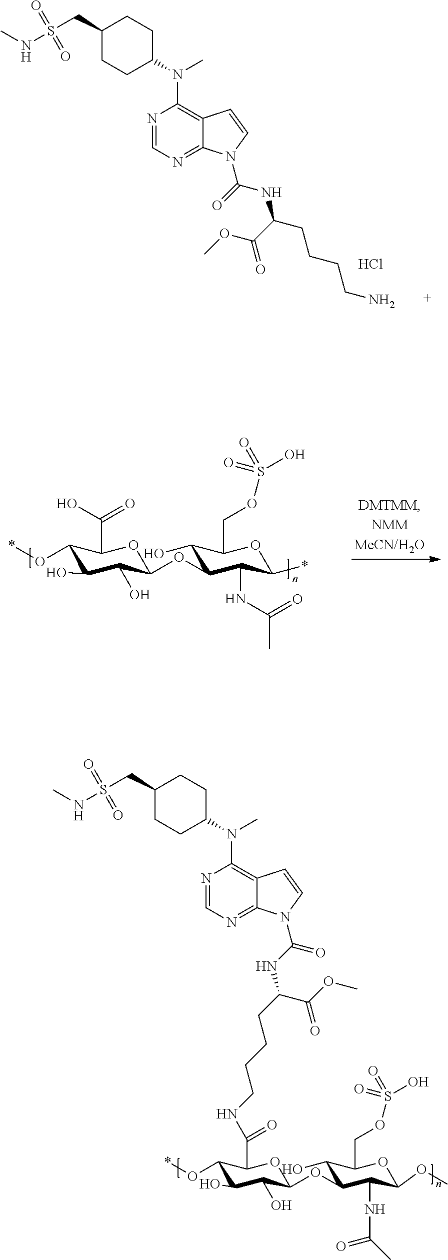

Chondroitin sulfate (331 mg, 0.72 mmol) was dissolved in 35 mL of deionized water and 20 mL of acetonitrile while stirring. To the solution was added methyl (2S)-6-amino-2-[[4-[methyl-[4-(methylsulfamoylmethyl)cyclohexyl]amino]pyrrolo[2,3-d]pyrimidine-7-carbonyl]amino]hexanoate (400 mg, 0.72 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 235 mg, 0.85 mmol) and stirred for 72 hours at room temperature. NaCl (421 mg, 7.2 mmol) was then added to the reaction mixture, which was stirred for 1 hour and followed by the dropwise addition of anhydrous alcohol (40 mL) while stirring. The mixture was filtered. The filter cake was washed with anhydrous alcohol, and dried under vacuum to give the title compound as a white solid (0.255 g, Yield: 35%, DSR: 5%). ¹H-NMR (400 MHz, D₂O): δ ppm 8.44-8.34 (m, 0.05H), 7.91-7.79 (m, 0.05H), 7.02-6.92 (m, 0.05H), 4.64-4.39 (m, 2.05H), 4.31-3.06 (m, 10.5H), 3.03-2.90 (m, 0.1H), 2.52-2.35 (m, 0.15H), 2.34-1.54 (m, 3.4H), 1.48-1.24 (m, 0.35H).

### Example 92

### Preparation of conjugate of 4-(((3R,4R)-1-(2-cyanoacetyl)-4-methylpiperidin-3-yl)(methyl) amino)-N-(4-(piperidin-4-yl)phenyl)-7H-pyrrolo[2,3-d]pyrimidine-7-carboxamide and HA

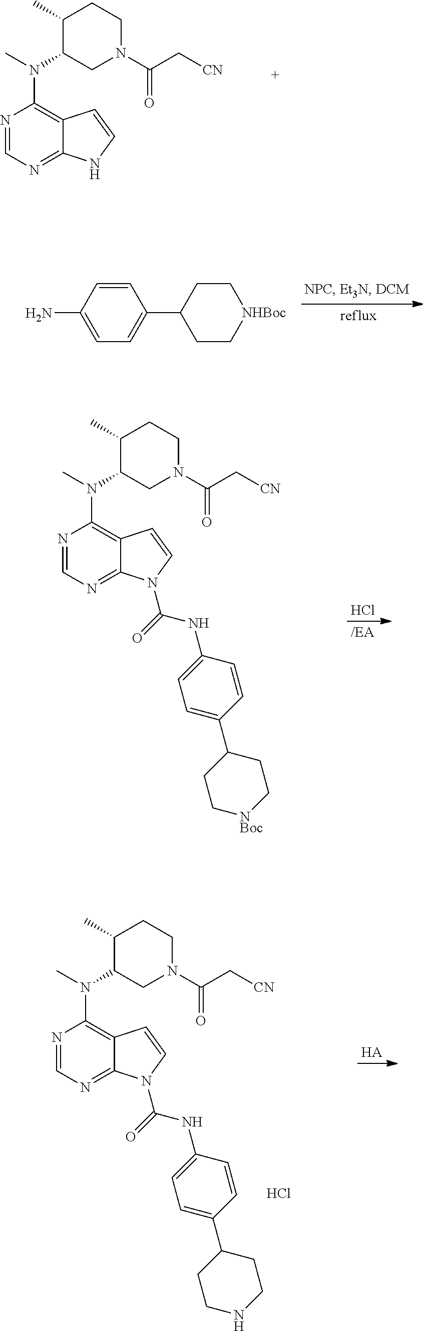

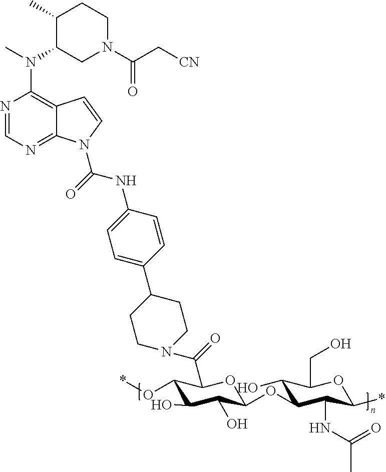

### Step 1: Preparation of tert-butyl 4-(4-(4-(((3R,4R)-1-(2-cyanoacetyl)-4-methylpiperidin-3-yl) (methyl)amino)-7H-pyrrolo[2,3-d]pyrimidine-7-carboxamido)phenyl)piperidine-1-carboxylate

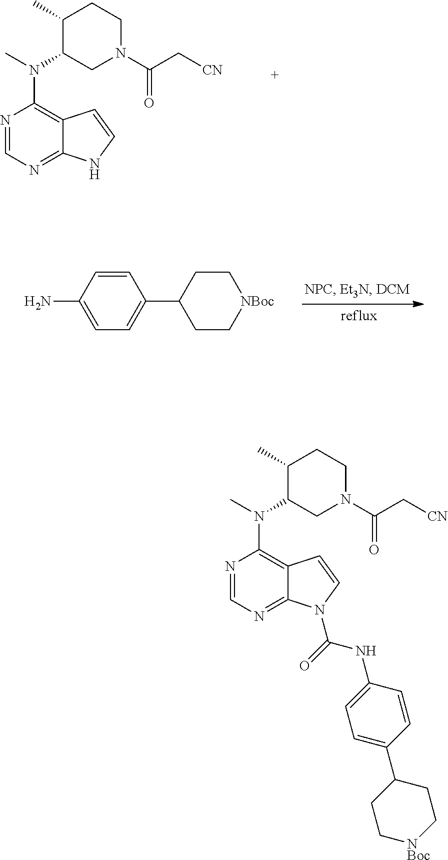

To a mixture of tofacitinib (1.8 g, 5.76 mmol, 1 eq) and bis(4-nitrophenyl) carbonate (1.93 g, 6.34 mmol, 1.1 eq) in DCM (30 mL) was added triethylamine (1.46 g, 14.4 mmol, 2.5 eq) under N₂ and the reaction mixture was heated to reflux for 3 hours. Then tert-butyl 4-(4-aminophenyl) piperidine-1-carboxylate (1.591 g, 5.76 mmol, 1 eq) was added and the resulting mixture was refluxed for 12 h. The solvent was removed under reduced pressure and the residue was purified by silical gel chromatography to give the title product (1.25 g, yield: 35.3%); MS (m/z): [M+H]+ calcd for C₃₃H₄₂N₈O₄, 615.33; found: 615.3.

### Step 2: Preparation of 4-(((3R,4R)-1-(2-cyanoacetyl)-4-methylpiperidin-3-yl)(methyl)amino)-N-(4-(piperidin-4-yl)phenyl)-7H-pyrrolo[2,3-d]pyrimidine-7-carboxamide hydrochloride

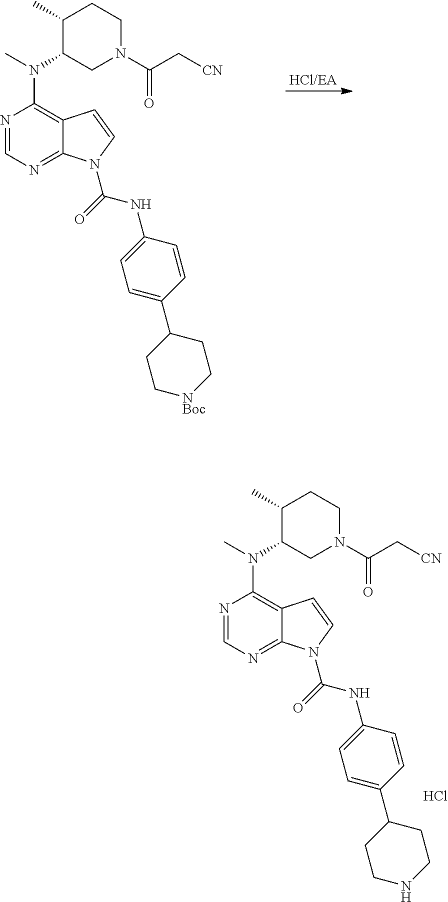 [SMILES: C[C@@H]1CCN(C(=O)CC#N)C[C@@H]1N(C)c1ncnc2c1ccn2C(=O)Nc1ccc(C2CCN(C(=O)OC(C)(C)C)CC2)cc1 ; C[C@@H]1CCN(C(=O)CC#N)C[C@@H]1N(C)c1ncnc2c1ccn2C(=O)Nc1ccc(C2CCNCC2)cc1 ; Cl]

To a solution of tert-butyl 4-(4-(4-(((3R,4R)-1-(2-cyanoacetyl)-4-methyl piperidin-3-yl) (methyl) amino)-7H-pyrrolo[2,3-d]pyrimidine-7-carboxamido)phenyl)piperidine-1-carboxylate (1 g, 3.18 mmol) in ethyl acetate (10 mL) was added 4M HCl in ethyl acetate solution (4 mL, 16 mmol) dropwise under N₂ at 0° C. The resulting reaction mixture was stirred at 0° C. for 30 min and then stirred at room temperature for 2 days. After the solvent was removed under reduced pressure, the resulting solid was stirred in ethyl acetate (24 mL) for 0.5 h, then filtered to give the desired product as solid (0.9 g, yield: 100%); MS (m/z): [M+H]⁺ calcd for C₂₈H₃₄N₈O₂, 515.28; found: 515.3.

### Step 3: Preparation of conjugate of 4-(((3R,4R)-1-(2-cyanoacetyl)-4-methylpiperidin-3-yl)(methyl) amino)-N-(4-(piperidin-4-yl)phenyl)-7H-pyrrolo[2,3-d]pyrimidine-7-carboxamide and HA

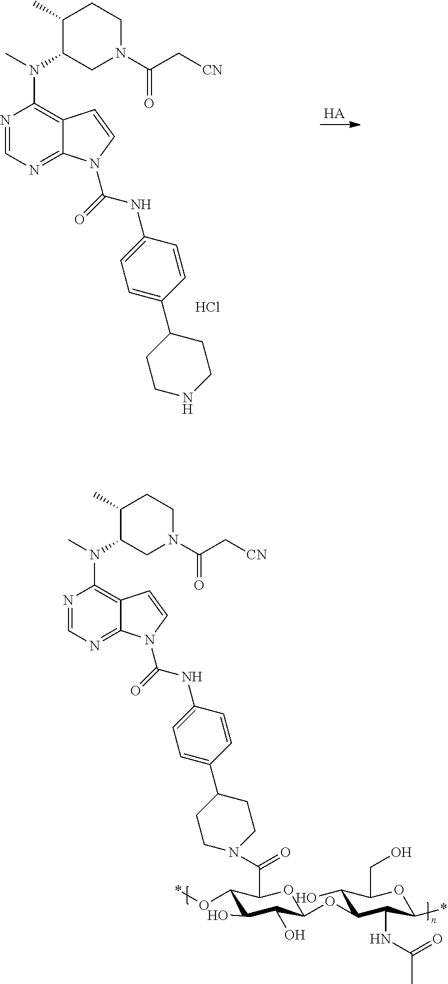

Sodium hyaluronate (0.174 g, 0.432 mmol) was dissolved in MeCN (22 ml) and H₂O (35 mL). Then 4-methylmorpholine (0.066 g, 0.65 mmol) and 2-chloro-4,6-dimethoxy-1,3,5-triazine (0.076 g, 0.432 mmol) were added into the above solution at 0° C. The resulting reaction mixture was stirred at 0° C. for 30 min, then warmed to room temperature and stirred for 1 h. Then 4-(((3R,4R)-1-(2-cyanoacetyl)-4-methylpiperidin-3-yl)(methyl)amino)-N-(4-(piperidin-4-yl)phenyl)-7H-pyrrolo[2,3-d]pyrimidine-7-carboxamide hydrochloride (0.238 g, 0.432 mmol) was added into the solution, and the resulting solution was stirred 3 days at room temperature. NaCl (253 mg, 4.32 mmol) in H₂O (1 ml) was added to the above reaction mixture and stirred for 0.5 h. Then acetone (350 ml) was added dropwise to the above mixture, while the precipitate was formed. The mixture was filtered and the cake was washed with acetone. The solid was dissolved in MeCN (20 ml) and H₂O (40 ml) to form a uniform solution and was Dialysis over a 3.5 kDa Mw cutoff membrane against deionized water, and then lyophilized to afford the title compound (0.15 g, yield: 43.2%, DS: 7.6%). ¹H-NMR (400 MHz, D₂O) δ ppm 7.49-7.29 (m, 0.53H), 4.70-4.20 (m, 2H), 4.00-3.23 (m, 9.14H), 2.50-2.30 (m, 0.19H), 1.99 (d, 3H), 1.60-1.40 (m, 0.51H), 1.3-1.15 (m, 0.2H), 1.15-0.85 (m, 0.2H).

With the Step 3 reaction conditions, sodium hyaluronate (0.174 g MW 2000 KDa) provided corresponding product (0.16 g, yield: 46%, DS=2.3%). ¹H-NMR (400 MHz, D₂O) δ ppm 7.75-7.15 (m, 0.16H), 4.50-4.25 (m, 2H), 4.15-2.90 (m, 10.34H), 2.45-2.35 (m, 0.09H), 1.99 (s, 3H), 1.25-1.15 (m, 0.05H), 1.05-0.95 (m, 0.1H).

### Example 93

### Preparation of conjugate of 4-(((3R,4R)-1-(2-cyanoacetyl)-4-methylpiperidin-3-yl)(methyl) amino)-N′-glycyl-7H-pyrrolo[2,3-d]pyrimidine-7-carbohydrazide and HA

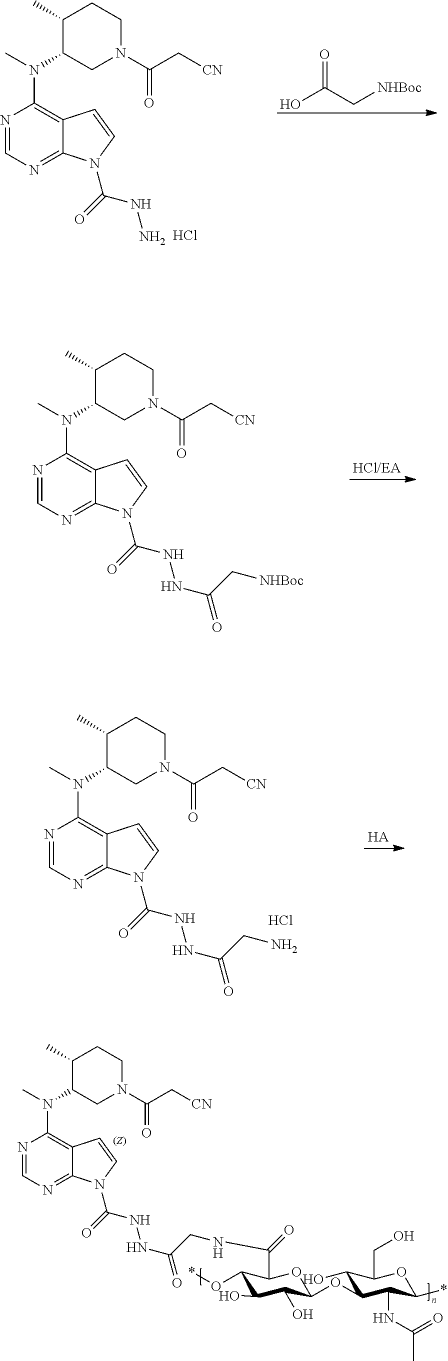

### Step 1: Preparation of tert-butyl (2-(2-(4-(((3R,4R)-1-(2-cyanoacetyl)-4-methylpiperidin-3-yl) (methyl)amino)-7H-pyrrolo[2,3-d]pyrimidine-7-carbonyl)hydrazinyl)-2-oxoethyl)carbamate

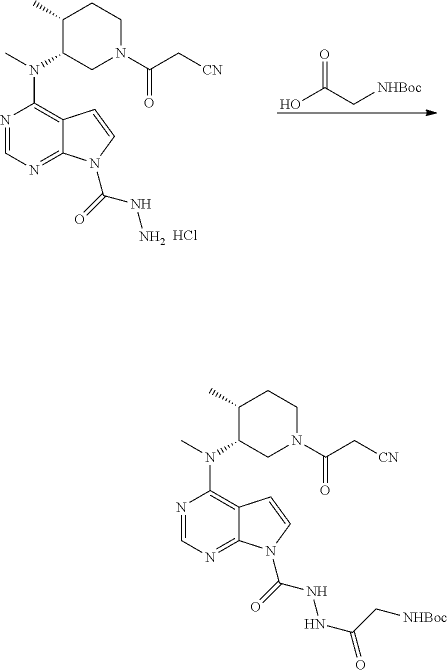 [SMILES: C[C@@H]1CCN(C(=O)CC#N)C[C@@H]1N(C)c1ncnc2c1ccn2C(=O)NN ; Cl ; CC(C)(C)OC(=O)NCC(=O)O ; C[C@@H]1CCN(C(=O)CC#N)C[C@@H]1N(C)c1ncnc2c1ccn2C(=O)NNC(=O)CNC(=O)OC(C)(C)C]

To a solution of (tert-butoxycarbonyl)glycine (0.414 g, 2.364 mmol) in MeCN (10 mL) was added HATU (0.898 g, 2.364 mmol), DIPEA (0.512 g, 3.94 mmol) at room temperature. The resulting reaction mixture was stirred at rt for 30 min, then 4-(((3R,4R)-1-(2-cyanoacetyl)-4-methyl piperidin-3-yl) (methyl)amino)-7H-pyrrolo[2,3-d] pyrimidine-7-carbohydrazide hydrochloride (0.8 g, 1.97 mmol, 1 eq) was added-into the above solution. The resulting solution was stirred at room temperature for 10 hs, then the solvent was removed under reduced pressure, the residue was purified by silical gel chromatography to give the title product (0.642 g, yield: 62%); MS (m/z): [M+H]⁺ calcd for C₂₄H₃₃N₉O₅, 528.26; found: 528.3.

### Step 2: Preparation of 4-(((3R,4R)-1-(2-cyanoacetyl)-4-methylpiperidin-3-yl)(methyl)amino)-N′-glycyl-7H-pyrrolo[2,3-d]pyrimidine-7-carbohydrazide hydrochloride

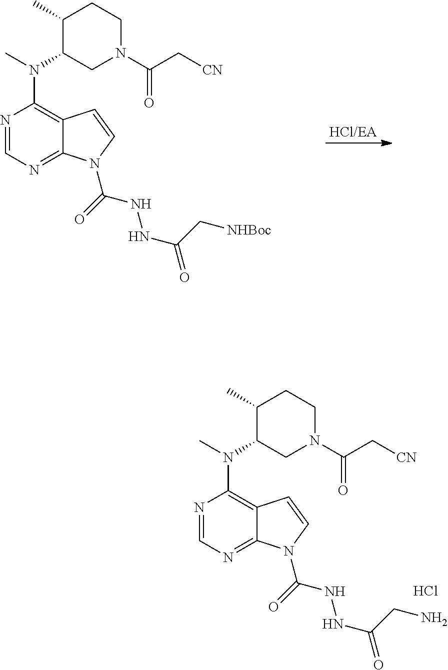 [SMILES: C[C@@H]1CCN(C(=O)CC#N)C[C@@H]1N(C)c1ncnc2c1ccn2C(=O)NNC(=O)CNC(=O)OC(C)(C)C ; C[C@@H]1CCN(C(=O)CC#N)C[C@@H]1N(C)c1ncnc2c1ccn2C(=O)NNC(=O)CN ; Cl]

To a solution of tert-butyl (2-(2-(4-(((3R,4R)-1-(2-cyanoacetyl)-4-methylpiperidin-3-yl) (methyl) amino)-7H-pyrrolo[2,3-d]pyrimidine-7-carbonyl)hydrazinyl)-2-oxoethyl)carbamate (0.642 g, 1.217 mmol) in ethyl acetate (10 mL) was added 4M HCl in ethyl acetate solution (4 mL, 16 mmol) dropwisely under N₂ at 0° C. The resulting reaction mixture was stirred at 0° C. for 30 min and then stirred at room temperature for 2 days. The solvent was removed under reduced pressure, the resulting solid was stirred in ethyl acetate (10 mL) for 0.5 h, then filtered to give the desired product as solid (0.56 g, yield: 100%); MS (m/z): [M+H]⁺ calcd for C₁₉H₂₅N₉O₃, 428.21; found: 428.2.

### Step 3: Preparation of conjugate of 4-(((3R,4R)-1-(2-cyanoacetyl)-4-methylpiperidin-3-yl)(methyl) amino)-N′-glycyl-7H-pyrrolo[2,3-d]pyrimidine-7-carbohydrazide and HA

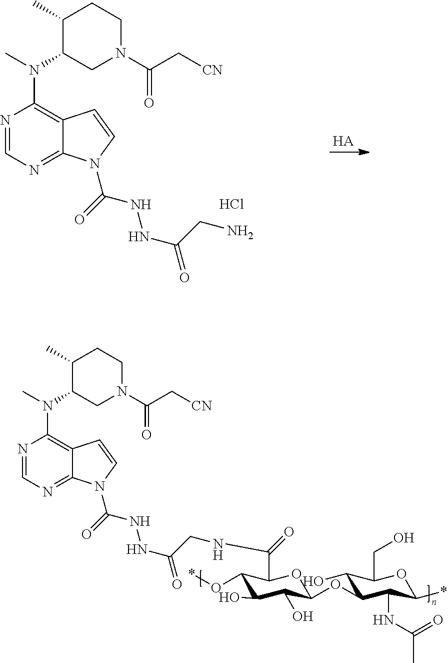

To a mixture of sodium hyaluronate (0.174 g, 0.431 mmol) in MeCN (22 ml) and H₂O (35 ml), 4-methylmorpholine (0.066 g, 0.65 mmol) and 2-chloro-4,6-dimethoxy-1,3,5-triazine (0.076 g, 0.431 mmol) were added at 0° C. The resulting reaction mixture was stirred at 0° C. for 30 min, and then stirred at room temperature for 1 h. Then 4-(((3R,4R)-1-(2-cyanoacetyl)-4-methylpiperidin-3-yl)(methyl)amino)-N′-glycyl-7H-pyrrolo[2,3-d]pyrimidine-7-carbohydrazide hydrochloride (0.2 g, 0.431 mmol) was added into the solution and was stirred for 3 days at room temperature. NaCl (253 mg) in H₂O (2 mL) was added and stirred for 0.5 h. Acetone (350 mL) was added dropwisely to the above mixture, while the precipitate was formed. The mixture was filtered, the cake was washed with acetone and then dissolved in MeCN (20 mL) and H₂O (40 mL) to form a uniform solution. Dialysis over a 3.5 kDa Mw cutoff membrane of the solution against deionized water, and then lyophilized afforded the title compound (0.183 g, yield: 49%, DS: 11%). ¹H-NMR (400 MHz, D₂O) δ ppm 8.90-7.40 (m, 0.11H), 7.75-7.35 (m, 0.11H), 6.95-6.50 (m, 0.11H), 4.75-4.20 (m, 2H), 4.00-3.23 (m, 11.36H), 2.60-2.40 (m, 0.11H), 1.99 (s, 3H), 1.80-1.65 (m, 0.22H), 1.15-0.85 (m, 0.33H).

With the Step 3 reaction condition, sodium hyaluronate (0.174 g MW 2000 KDa) provided corresponding product (0.19 g, yield: 51%, DS=11%). ¹H-NMR (400 MHz, D₂O) δ ppm 8.30-7.90 (m, 0.11H), 7.65-7.20 (m, 0.11H), 6.9-6.30 (m, 0.11H), 4.60-4.20 (m, 2H), 4.00-3.00 (m, 11.36H), 2.50-2.30 (m, 0.11H), 1.99 (s, 3H), 1.70-1.60 (m, 0.22H), 1.10-0.85 (m, 0.33H).

### Example 94

### Preparation of conjugate of phenyl N-(4-aminobutyl)-P-(4-(((3R,4R)-1-(2-cyanoacetyl)-4-methylpiperidin-3-yl)(methyl) amino)-7H-pyrrolo[2,3-d]pyrimidin-7-yl)phosphonamidate and HA

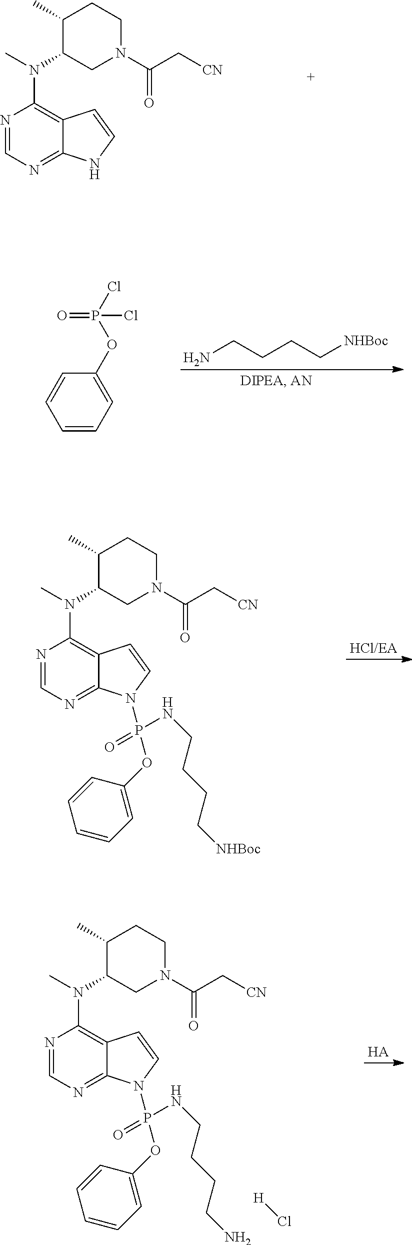

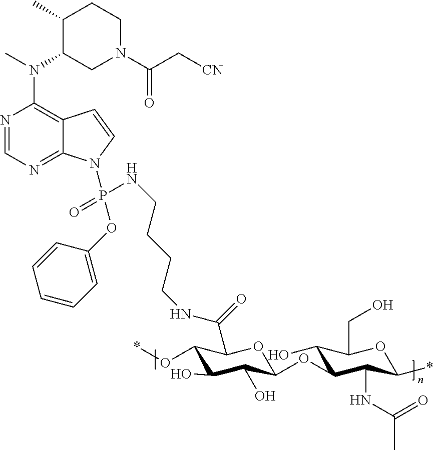

### Step 1: Preparation of tert-butyl (4-(((4-(((3R,4R)-1-(2-cyanoacetyl)-4-methylpiperidin-3-yl)(methyl)amino)-7H-pyrrolo[2,3-d]pyrimidin-7-yl)(phenoxy)phosphoryl)amino)butyl)carbamate

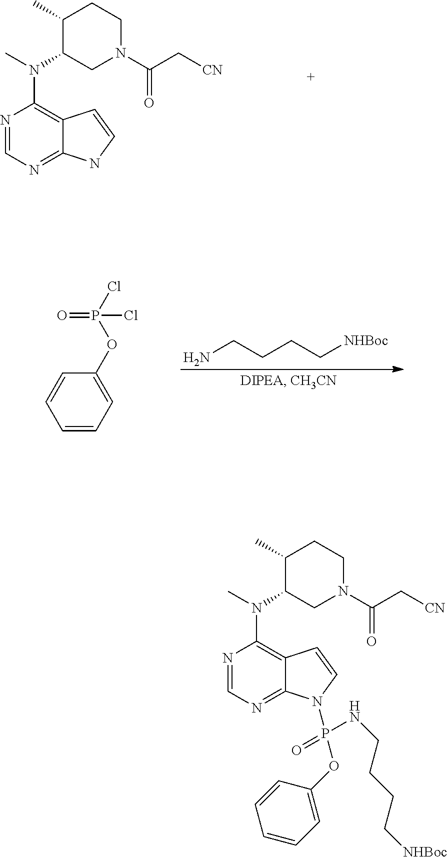 [SMILES: C[C@@H]1CCN(C(=O)CC#N)C[C@@H]1N(C)c1ncnc2[nH]ccc12 ; O=P(Cl)(Cl)Oc1ccccc1 ; CC(C)(C)OC(=O)NCCCCN ; C[C@@H]1CCN(C(=O)CC#N)C[C@@H]1N(C)c1ncnc2c1ccn2P(=O)(NCCCCNC(=O)OC(C)(C)C)Oc1ccccc1]

To a mixture of tofacitinib (1 g, 3.2 mmol), tert-butyl (4-aminobutyl) carbamate (603 mg, 3.2 mmol) and phenyl phosphorodichloridate (675 mg, 3.2 mmol) in MeCN (20 ml) was added N,N-Diisopropylethylamine (1.45 g, 16 mmol) under N2, the reaction mixture was heated to reflux for 16 hours. After the solvent was removed under reduced pressure, the residue was purified by silical gel chromatography to give the title product. (400 mg, yield: 19.6%); MS (m/z): [M+H]⁺ calcd for C₃₁H₄₃N₈O₅P, 639.31; found: 639.3.

### Step 2: Preparation of Phenyl N-(4-aminobutyl)-P-(4-(((3R,4R)-1-(2-cyanoacetyl)-4-methylpiperidin-3-yl)(methyl)amino)-7H-pyrrolo[2,3-d]pyrimidin-7-yl)phosphonamidate hydrochloride

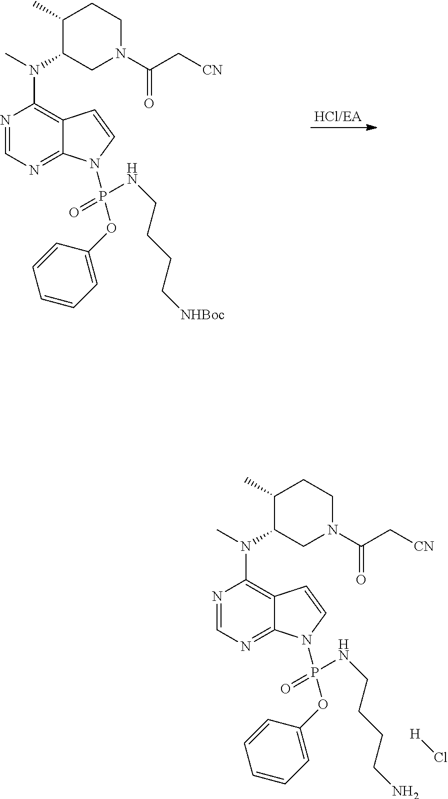

To a solution of tert-butyl (4-(((4-(((3R,4R)-1-(2-cyanoacetyl)-4-methylpiperidin-3-yl) (methyl)amino)-7H-pyrrolo[2,3-d]pyrimidin-7-yl)(phenoxy)phosphoryl)amino)butyl)carbamat (200 mg, 0.313 mmol) in EA (10 ml) was added 4.5 M HCl in EA (0.9 ml, 4.5 mmol) dropwisely under N₂ at 0° C. The resulting reaction mixture was stirred at 0° C. for 30 min, and then stirred at room temperature for 2 days. After solvent was removed under reduced pressure, the resulting solid was stirred in EA (20 ml) for 0.5 h, then filtered to give the desired product (150 mg, yield: 83.4%); MS (m/z): [M+H]⁺ calcd for C₂₆H₃₅N₈O₃P, 539.26; found: 539.3.

### Step 3: Preparation of conjugate of phenyl N-(4-aminobutyl)-P-(4-(((3R,4R)-1-(2-cyanoacetyl)-4-methylpiperidin-3-yl)(methyl) amino)-7H-pyrrolo[2,3-d]pyrimidin-7-yl)phosphonamidate and HA

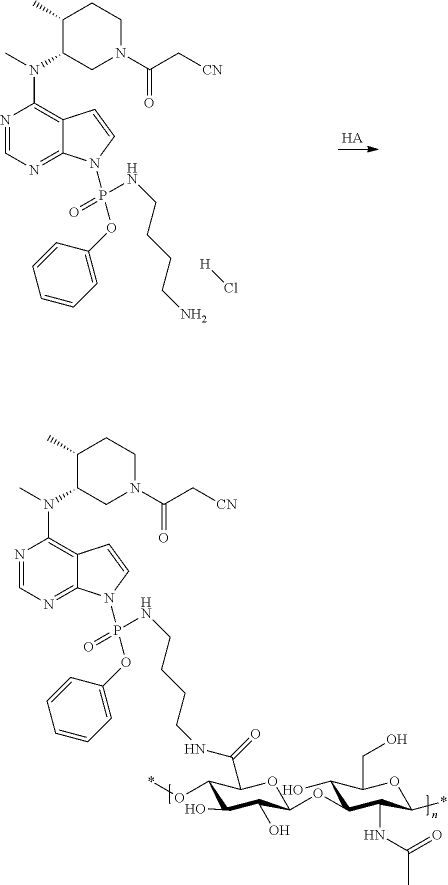

To a mixture of sodium hyaluronate (84 mg, 0.21 mmol) in MeCN (15.6 ml) and H₂O (24 ml), were added 4-methylmorpholine (31.8 mg, 0.31 mmol), 2-chloro-4,6-dimethoxy-1,3,5-triazine (36.6 mg, 0.21 mmol) at 0° C. The resulting reaction mixture was stirred at 0° C. for 30 min, and stirred at room temperature for 1 h. Then phenyl N-(4-aminobutyl)-P-(4-(((3R,4R)-1-(2-cyanoacetyl)-4-methylpiperidin-3-yl) (methyl)amino)-7H-pyrrolo[2,3-d]pyrimidin-7-yl)phosphonamidate hydrochloride (120 mg, 0.21 mmol) was added into the solution, and the pH of the reaction mixture was adjust between 6.5 to 7 with N-MM. The resulting solution was stirred 3 days at room temperature. NaCl (122 mg) in H₂O (1 mL) was added and stirred for 0.5 h. Then acetone (168 mL) was added dropwisely and the resulting mixture was filtered. The cake was washed with acetone and dissolved in MeCN (8.4 mL) and H₂O (16.8 mL) to form a uniform solution. Dialysis of the solution against deionized water for 3 times, then lyophilization afforded the title compound (82 mg, yield: 43.2%, DS=31%). ¹H-NMR (400 MHz, D₂O) δ 8.35-8.25 (m, 0.28H), 7.55-7.35 (m, 0.86H), 7.30-7.15 (m, 0.94H), 6.90-6.80 (m, 0.42H), 4.60-4.20 (m, 2H), 4.10-3.20 (m, 15.37H), 2.65-2.55 (m, 0.53H), 1.99 (s, 3H), 1.75-1.40 (m, 2H), 1.20-1.15 (m, 0.95H).

### Example 95

### Preparation of conjugate of methyl (Z)-1-glycyl-3-(((4-(N-methyl-2-(4-methylpiperazin-1-yl)acetamido)phenyl)amino)(phenyl)methylene)-2-oxoindoline-6-carboxylate and HA

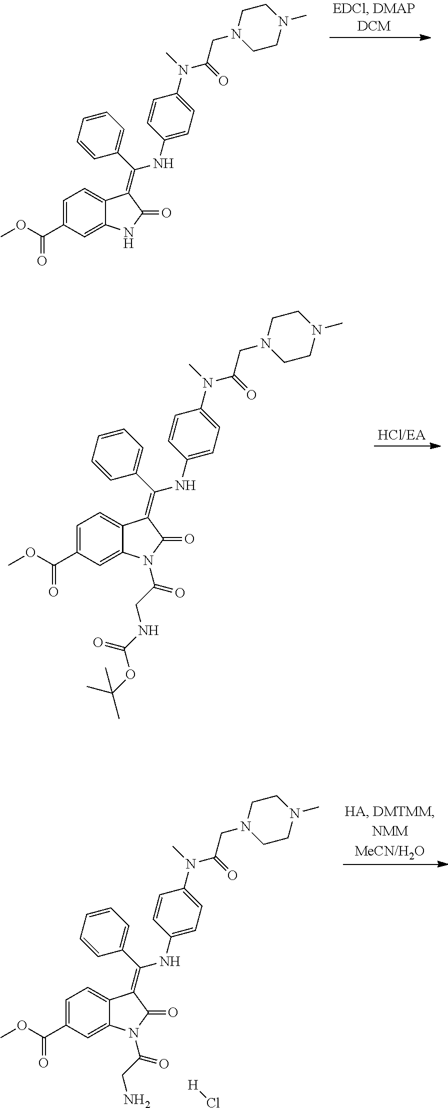

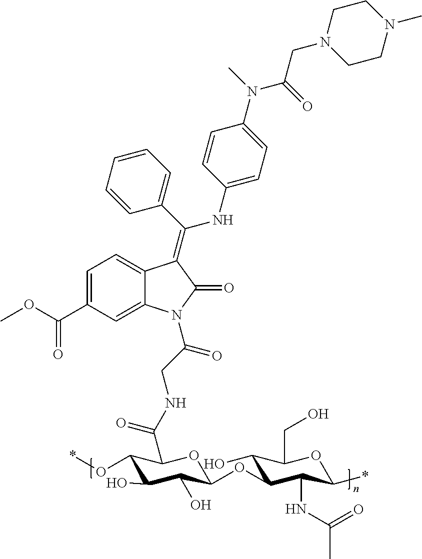

### Step 1: Preparation of methyl (Z)-1-((tert-butoxycarbonyl)glycyl)-3-(((4-(N-methyl-2-(4-methylpiperazin-1-yl)acetamido)phenyl)amino)(phenyl)methylene)-2-oxoindoline-6-carboxylate

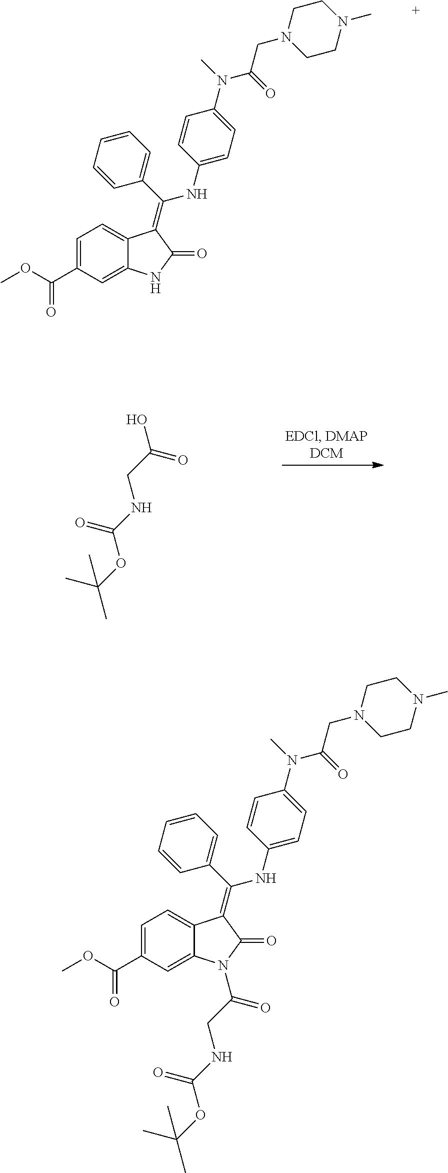

methyl (3Z)-3-[[4-[methyl-[2-(4-methylpiperazin-1-yl)acetyl]amino]anilino]-phenyl-methylene]-2-oxo-indoline-6-carboxylate (1079 mg, 2 mmol), (tert-butoxycarbonyl)glycine (701 mg, 4 mmol), 1-ethyl-3-(3-dimethylaminopropyl)carbodiimide (EDCI, 864.5 mg, 4.5 mmol), N,N-dimethylpyridin-4-amine (DMAP, 1098 mg, 9 mmol) were dissolved in DCM (80 mL). The reaction mixture was stirred at room temperature for 16 hours. The solution was diluted with DCM and washed with water and saturated brine solution. The organic layer was dried over sodium sulfate, filtered and then concentrated under reduced pressure. The crude residue was purified by column chromatography (DCM/MeOH=50:1-20:1) to afford the title compound (700 mg, Yield: 50.2%). MS (m/z): [M+H]⁺ calcd for C₃₈H₄₄N₆O₇, 697.81; found: 697.2. ¹H NMR (400 MHz, Chloroform-d) δ 12.11 (s, 1H), 8.91 (d, J=1.7 Hz, 1H), 7.64-7.50 (m, 4H), 7.42-7.37 (m, 2H), 7.06-7.00 (m, 2H), 6.82 (d, J=8.4 Hz, 2H), 5.92 (d, J=8.3 Hz, 1H), 5.45 (s, 1H), 4.83 (d, J=5.4 Hz, 2H), 3.87 (s, 3H), 3.19 (s, 3H), 2.96 (q, J=7.3 Hz, 2H), 2.79 (s, 2H), 2.56-2.34 (m, 6H), 2.28 (s, 3H), 1.50 (s, 9H).

### Step 2: Preparation of methyl (Z)-1-glycyl-3-(((4-(N-methyl-2-(4-methylpiperazin-1-yl)acetamido)phenyl)amino)(phenyl)methylene)-2-oxoindoline-6-carboxylate hydrochloride

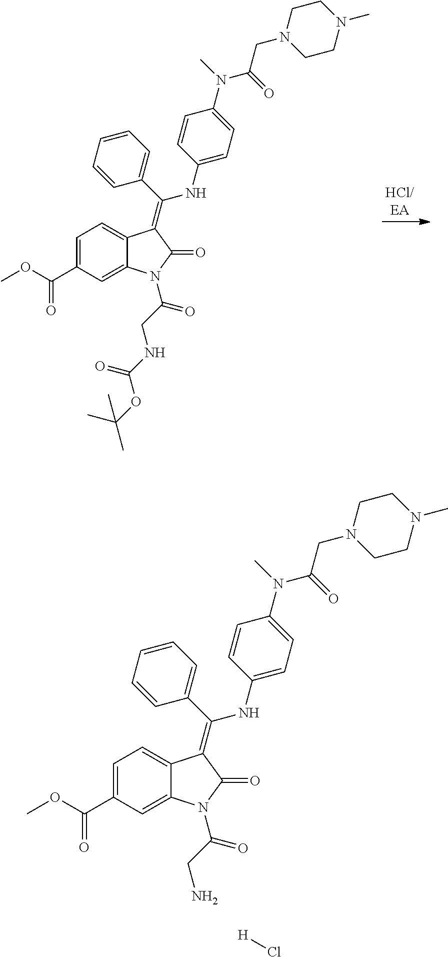

To a solution of methyl (Z)-1-((tert-butoxycarbonyl)glycyl)-3-(((4-(N-methyl-2-(4-methylpiperazin-1-yl)acetamido)phenyl)amino)(phenyl)methylene)-2-oxoindoline-6-carboxylate (139 mg, 0.2 mmol) in ethyl acetate (10 mL) was added 4M HCl in ethyl acetate solution (2 mL, 8 mmol) dropwisely under N₂ at 0° C. The resulting reaction mixture was stirred at 0° C. for 30 min and then stirred at room temperature for 20 hours. The solution was diluted with ethyl acetate (20 mL) and concentrated under reduced pressure to afford the title compound as a white solid (83 mg, Yield: 70%) that was used without further purification. MS (m/z): [M+H]+ calcd for C₃₃H₃₆N₆O₅, 597.69; found: 597.2.

### Step 3: Preparation of conjugate of methyl (Z)-1-glycyl-3-(((4-(N-methyl-2-(4-methylpiperazin-1-yl)acetamido)phenyl)amino)(phenyl)methylene)-2-oxoindoline-6-carboxylate and HA

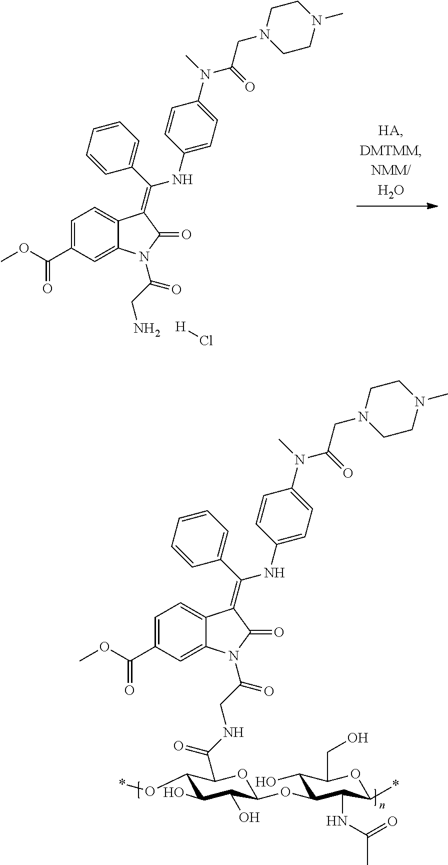

To a solution of sodium hyaluronate (201 mg, 0.5 mmol) in deionized water (40 ml) and acetonitrile (26 ml) was added methyl (Z)-1-glycyl-3-(((4-(N-methyl-2-(4-methylpiperazin-1-yl)acetamido)phenyl)amino)(phenyl)methylene)-2-oxoindoline-6-carboxylate (149 mg, 0.25 mmol) and 4-methylmorpholine (NMM, 25 mg, 0.25 mmol) at room temperature. The reaction mixture was stirred at room temperature for 2 hours. To the solution, 4-(4,6-dimethoxy-1,3,5-triazin-2-yl)-4-methylmorpholinium chloride (DMTMM, 104 mg, 0.75 mmol) was added and stirred for 72 hours at room temperature. NaCl (293 mg) was added to the reaction mixture and was stirred for 1 hour and then followed by the dropwise addition of acetone (200 mL). The mixture was filtered. The filter cake was washed with acetone, and dried under vacuum to give the title compound as a white solid. (250 mg, yield: 52.2%, DS=17%). ¹H NMR (400 MHz, D₂O) δ 8.58-8.40 (m, 0.17H), 7.65-7.15 (m, 1.02H), 7.07-6.75 (m, 0.68H), 4.52-4.21 (m, 2H), 4.01-2.50 (m, 12.89H), 2.33-2.13 (m, 0.51H), 2.00-1.59 (m, 3H)

### Example 96

### Preparation of conjugate of (Z)—N-(2-(diethylamino)ethyl)-5-((5-fluoro-1-(hydrazinecarbonyl)-2-oxoindolin-3-ylidene)methyl)-2,4-dimethyl-1H-pyrrole-3-carboxamide and HA

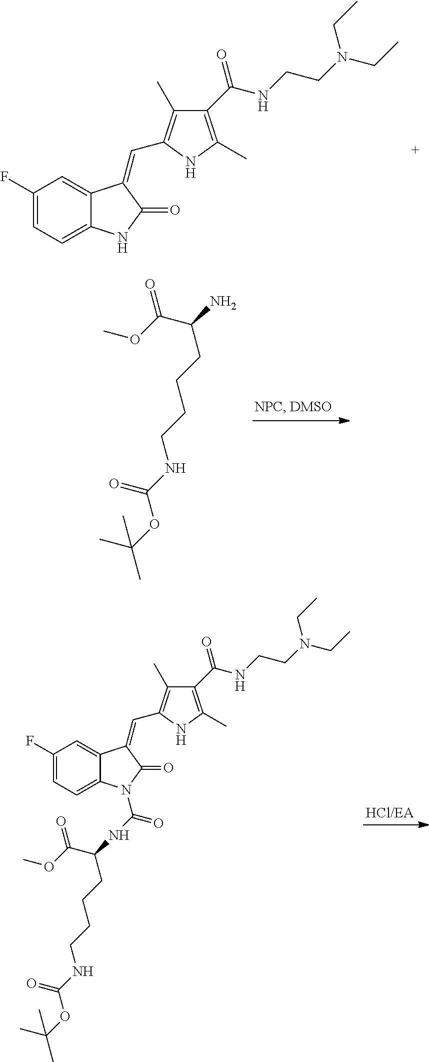

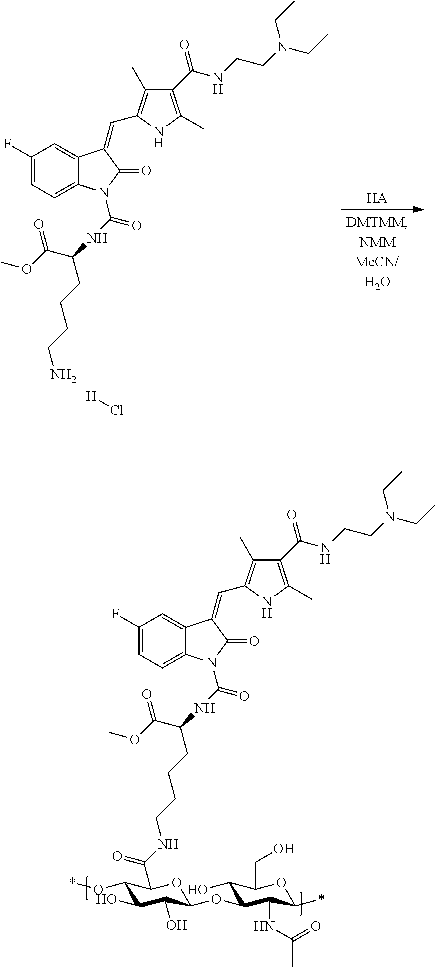

### Step 1: Preparation of methyl (Z)—N⁶-(tert-butoxycarbonyl)-N2-(3-((4-((2-(diethylamino)ethyl)carbamoyl)-3,5-dimethyl-1H-pyrrol-2-yl)methylene)-5-fluoro-2-oxoindoline-7-carbonyl)-L-lysinate

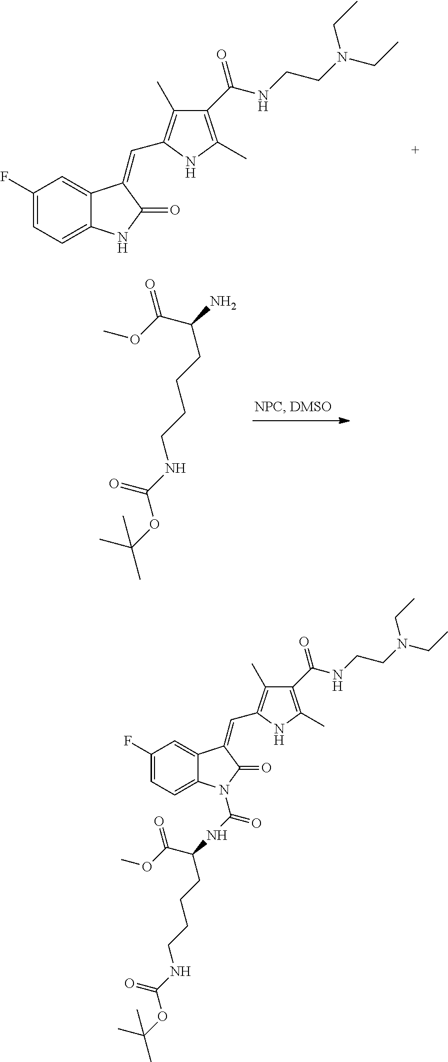

A mixture of (Z)—N-(2-(diethylamino)ethyl)-5-((5-fluoro-2-oxoindolin-3-ylidene)methyl)-2,4-dimethyl-1H-pyrrole-3-carboxamide (1594 mg, 4 mmol) and bis(4-nitrophenyl) carbonate (1460 mg, 4.8 mmol) in DMSO (30 mL) was stirred at room temperature for 7 hours. Then methyl N6-(tert-butoxycarbonyl)-L-lysinate (1250 mg, 4.8 mmol) was added. The reaction mixture was stirred at 40° C. for 16 hours. The reaction mixture was poured into water and extracted with EtOAc. The organic layer was dried over sodium sulfate, filtered and then concentrated under reduced pressure. The crude residue was purified by column chromatography (DCM:Methanol=20:1) to afford the title compound 0.77 g, Yield: 28.10%). MS (m/z): [M+H]+ calcd for C₃₅H₄₉FN₆O₇, 685.81; found: 685.2. ¹H NMR (400 MHz, Chloroform-d) δ ppm 12.75 (s, 1H), 9.31 (d, J=7.6 Hz, 1H), 8.17 (d, J=4.7 Hz, 1H), 7.42 (s, 1H), 7.17 (dd, J=8.4, 2.5 Hz, 1H), 6.91 (td, J=9.0, 2.6 Hz, 1H), 6.63 (s, 1H), 4.75-4.64 (m, 1H), 4.60 (s, 1H), 3.80 (d, J=6.4 Hz, 3H), 3.52 (dd, J=10.9, 5.2 Hz, 2H), 3.14 (d, J=5.5 Hz, 2H), 2.76-2.46 (m, 12H), 2.08-1.79 (m, 2H), 1.60-1.37 (m, 13H), 1.06 (t, J=7.1 Hz, 6H).

### Step 2: Preparation of methyl (Z)-(3-((4-((2-(diethylamino)ethyl)carbamoyl)-3,5-dimethyl-1H-pyrrol-2-yl)methylene)-5-fluoro-2-oxoindoline-1-carbonyl)-L-lysinate hydrochloride

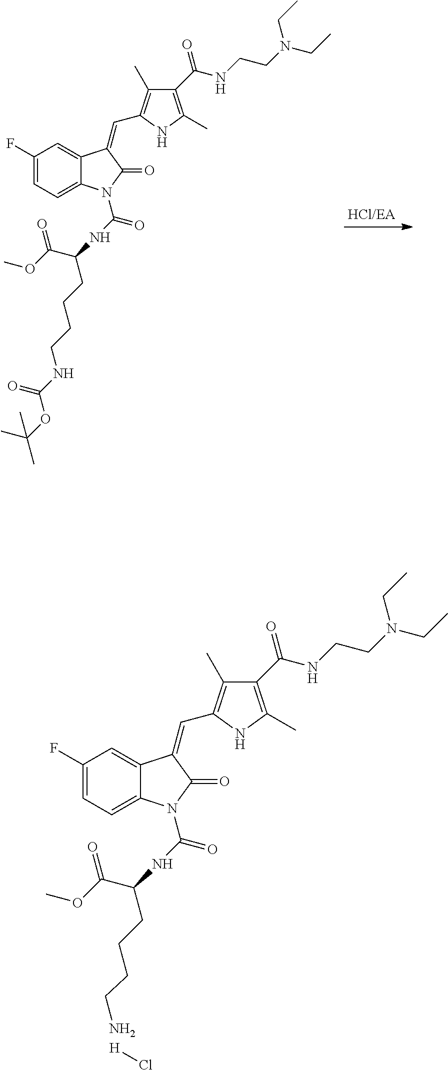

To a solution of methyl (Z)—N⁶-(tert-butoxycarbonyl)-N2-(3-((4-((2-(diethylamino)ethyl)carbamoyl)-3,5-dimethyl-1H-pyrrol-2-yl)methylene)-5-fluoro-2-oxoindoline-1-carbonyl)-L-lysinate (600 mg, 0.877 mmol) in ethyl acetate (18 mL) was added 4M HCl in ethyl acetate solution (3.6 mL, 14.4 mmol) dropwisely under N₂ at 0° C. The resulting reaction mixture was stirred at 0° C. for 30 min and then stirred at room temperature for 20 hours. The solution was diluted with ethyl acetate (20 mL) and concentrated under reduced pressure to afford the title compound as a solid (498 mg, Yield: 91.5%). MS (m/z): [M+H]+ calcd for C₃₀H₄₁FN₆O₅, 585.69; found: 585.2.

### Step 3: Preparation of conjugate of methyl (Z)-(3-((4-((2-(diethylamino)ethyl)carbamoyl)-3,5-dimethyl-1H-pyrrol-2-yl)methylene)-5-fluoro-2-oxoindoline-1-carbonyl)-L-lysinate and HA

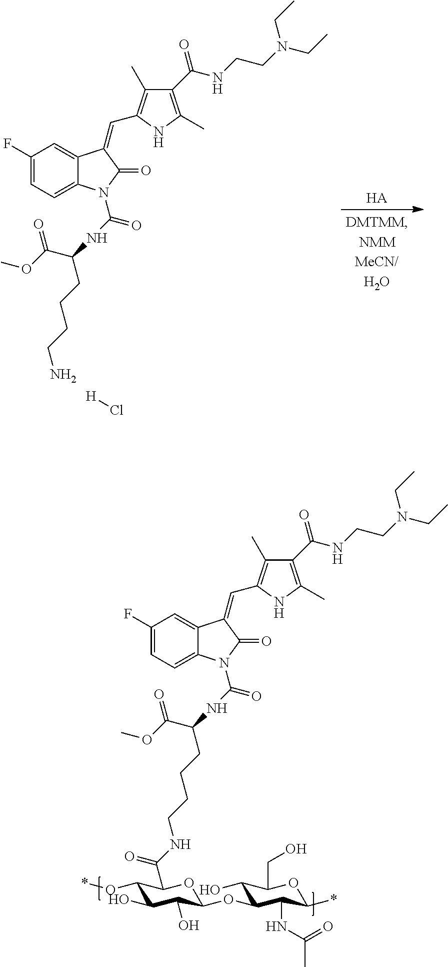

To the solution of sodium hyaluronate (130 mg, 0.21 mmol) in deionized water (24 mL) and acetonitrile (16 mL), were added methyl (Z)-(3-((4-((2-(diethylamino)ethyl)carbamoyl)-3,5-dimethyl-1H-pyrrol-2-yl)methylene)-5-fluoro-2-oxoindoline-1-carbonyl)-L-lysinatehydrochloride (130 mg, 0.21 mmol) and 4-methylmorpholine (NMM, 21 mg, 0.21 mmol) at room temperature. The reaction mixture was stirred at room temperature for 2 hours. And 4-(4,6-dimethoxy-1,3,5-triazin-2-yl)-4-methylmorpholinium chloride (DMTMM, 83 mg, 0.3 mmol) was added and stirred for 72 hours at room temperature. NaCl (176 mg) was then added to the reaction mixture and stirred for 1 hour and followed by the dropwise addition of acetone (40 mL). the mixture was filtered and filter cake was washed with acetone, and dried under vacuum to give the title compound as a white solid. (Sodium hyaluronate MW 50 KDa, 0.19 g, Yield: 95.7%, DS: 6%); ¹H NMR (400 MHz, D₂O) δ 7.82-7.62 (m, 0.06H), 7.38-7.13 (m, 0.06H), 6.97-6.72 (m, 0.12H), 4.55-4.27 (m, 2.06H), 4.06-2.75 (m, 10.42H), 2.53-1.48 (m, 4.08H), 1.39-1.26 (m, 0.06H)

### Example 97

### Preparation of conjugate of (Z)—N-(2-(diethylamino)ethyl)-5-((5-fluoro-1-(hydrazinecarbonyl)-2-oxoindolin-3-ylidene)methyl)-2,4-dimethyl-1H-pyrrole-3-carboxamide and HA

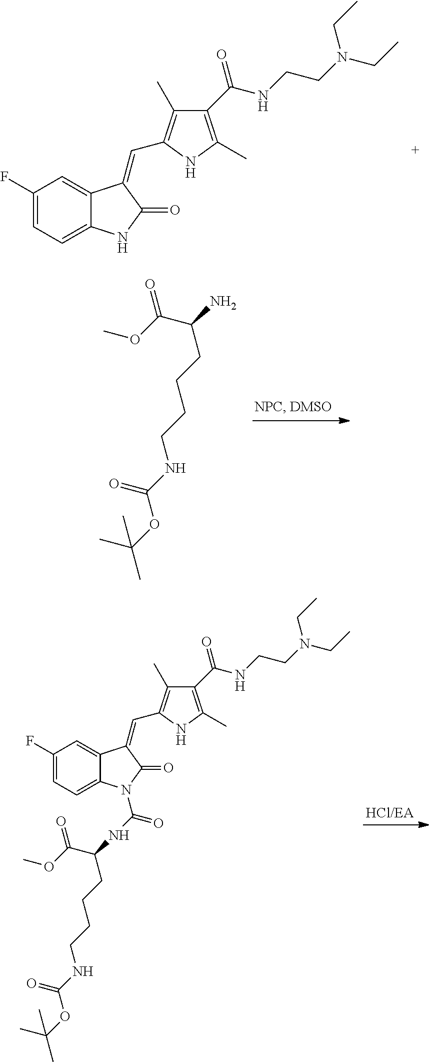

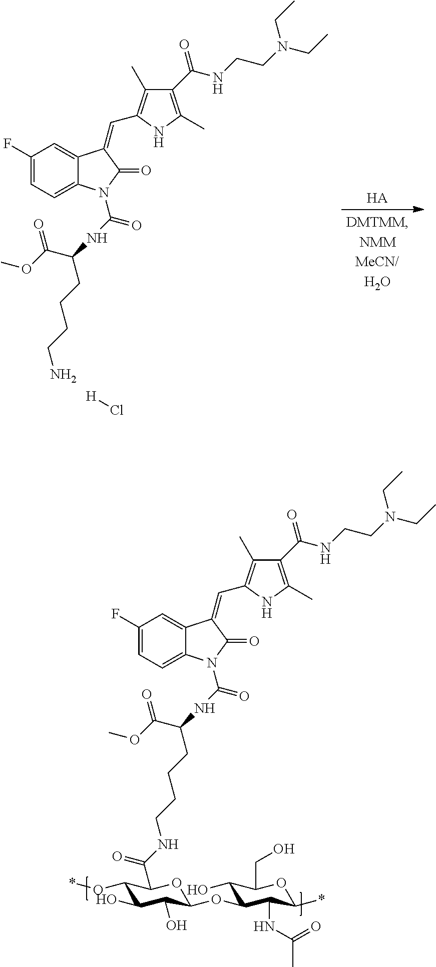

### Step 1: Preparation of tert-butyl (Z)-2-(3-((4-((2-(diethylamino)ethyl)carbamoyl)-3,5-dimethyl-1H-pyrrol-2-yl)methylene)-5-fluoro-2-oxoindoline-4-carbonyl)hydrazine-7-carboxylate

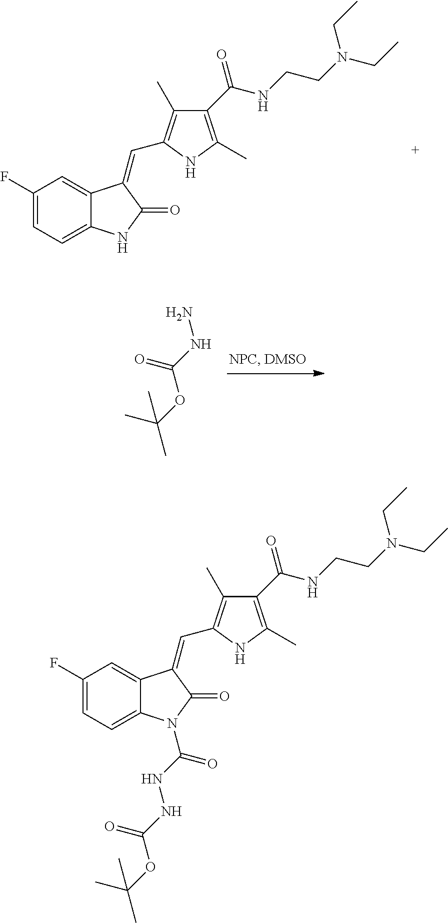

A mixture of (Z)—N-(2-(diethylamino)ethyl)-5-((5-fluoro-2-oxoindolin-3-ylidene)methyl)-2,4-dimethyl-1H-pyrrole-3-carboxamide (1594 mg, 4 mmol) and bis(4-nitrophenyl) carbonate (1460 mg, 4.8 mmol) in DMSO (30 mL) was stirred at room temperature for 7 hours. Then tert-butyl hydrazinecarboxylate (635 mg, 4.8 mmol) was added and the reaction mixture was stirred at 40° C. for 16 hours. The reaction mixture was poured into water and extracted with EtOAc. The organic layer was dried over sodium sulfate, filtered and then concentrated under reduced pressure. The crude residue was purified by column chromatography (DCM:Methanol=40:1) to afford the title compound (1.06 g, Yield: 47.60%). MS (m/z): [M+H]+ calcd for C₂₈H₃₇FN₆O₅, 557.64; found: 557.2. ¹H NMR (400 MHz, Chloroform-d) δ ppm 12.62 (s, 1H), 10.16 (s, 1H), 8.07 (s, 1H), 7.25 (d, J=10.1 Hz, 2H), 7.07 (d, J=8.2 Hz, 1H), 6.87 (t, J=8.5 Hz, 1H), 6.57 (s, 1H), 3.51 (q, J=5.3 Hz, 2H), 2.68 (t, J=5.9 Hz, 2H), 2.60 (dd, J=15.2, 8.0 Hz, 7H), 2.43 (s, 3H), 1.53 (s, 9H), 1.05 (t, J=7.1 Hz, 6H).

### Step 2: Preparation of (Z)—N-(2-(diethylamino)ethyl)-5-((5-fluoro-1-(hydrazinecarbonyl)-2-oxoindolin-3-ylidene)methyl)-2,4-dimethyl-1H-pyrrole-3-carboxamide hydrochloride

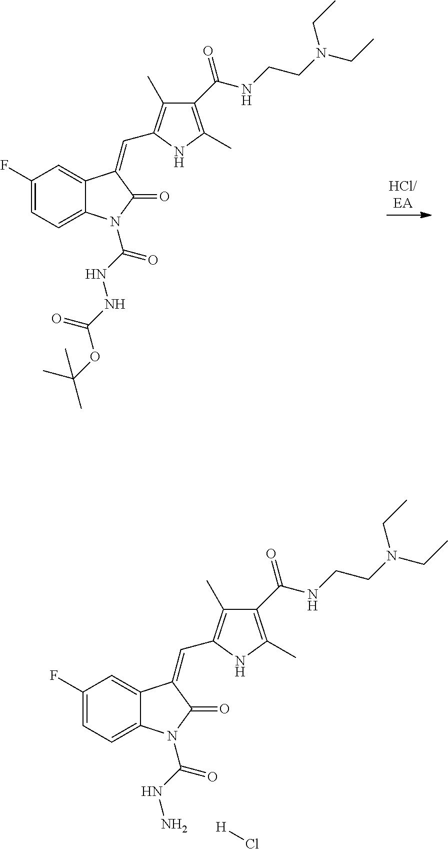 [SMILES: CCN(CC)CCNC(=O)c1c(C)[nH]c(/C=C2\C(=O)N(C(=O)NNC(=O)OC(C)(C)C)c3ccc(F)cc32)c1C ; CCN(CC)CCNC(=O)c1c(C)[nH]c(/C=C2\C(=O)N(C(=O)NN)c3ccc(F)cc32)c1C ; Cl]

To a solution of tert-butyl (Z)-2-(3-((4-((2-(diethylamino)ethyl)carbamoyl)-3,5-dimethyl-1H-pyrrol-2-yl)methylene)-5-fluoro-2-oxoindoline-1-carbonyl)hydrazine-1-carboxylate (1000 mg, 1.8 mmol) in ethyl acetate (40 mL) was added 4M HCl in ethyl acetate solution (8 mL, 32 mmol) dropwisely under N₂ at 0° C. The resulting reaction mixture was stirred at 0° C. for 30 min and then stirred at room temperature for 20 hours. The solution was diluted with ethyl acetate (20 mL) and concentrated under reduced pressure to afford the title compound as a white solid (410 mg, Yield: 82.8%) that was used without further purification. MS (m/z): [M+H]+ calcd for C₂₃H₂₉FN₆O₃, 457.52; found: 457.2.

### Step 3: Preparation of conjugate of (Z)—N-(2-(diethylamino)ethyl)-5-((5-fluoro-1-(hydrazinecarbonyl)-2-oxoindolin-3-ylidene)methyl)-2,4-dimethyl-1H-pyrrole-3-carboxamide and HA

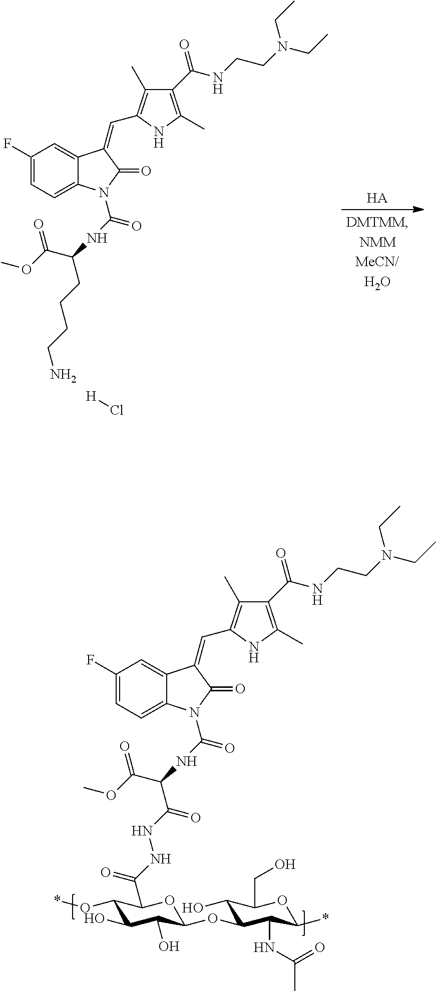

To a solution of sodium hyaluronate (161 mg, 0.4 mmol) in deionized water (32 mL) and acetonitrile (21 mL) were added (Z)—N-(2-(diethylamino)ethyl)-5-((5-fluoro-1-(hydrazinecarbonyl)-2-oxoindolin-3-ylidene)methyl)-2,4-dimethyl-1H-pyrrole-3-carboxamidehydrochloride (138 mg, 0.28 mmol) and 4-methylmorpholine (NMM, 28 mg, 0.28 mmol) at room temperature. The reaction mixture was stirred at room temperature for 2 hours and then 4-(4,6-dimethoxy-1,3,5-triazin-2-yl)-4-methylmorpholinium chloride (DMTMM, 111 mg, 0.4 mmol) was added and stirred for 72 hours at room temperature. NaCl (234 mg) was added to the reaction mixture and stirred for 1 hour and then followed by the dropwise addition of acetone (40 mL) with stirring. The mixture was filtered and filter cake was washed with acetone, and dried under vacuum to give the title compound as a white solid. (Sodium hyaluronate MW 50 KDa, 0.185 g, Yield: 56.6%, DS: 10%); ¹H NMR (400 MHz, D₂O) δ 7.76-7.19 (m, 0.20H), 7.08-6.66 (m, 0.20H), 4.56-4.24 (m, 2H), 4.07-2.88 (m, 10.20H), 2.54-1.55 (m, 4.20H), 1.41-1.22 (m, 0.60H).

### Example 98

### Preparation of conjugate of (Z)—N-(2-(diethylamino)ethyl)-5-((5-fluoro-2-oxo-1-(piperazine-1-carbonyl)indolin-3-ylidene)methyl)-2,4-dimethyl-1H-pyrrole-3-carboxamide and HA

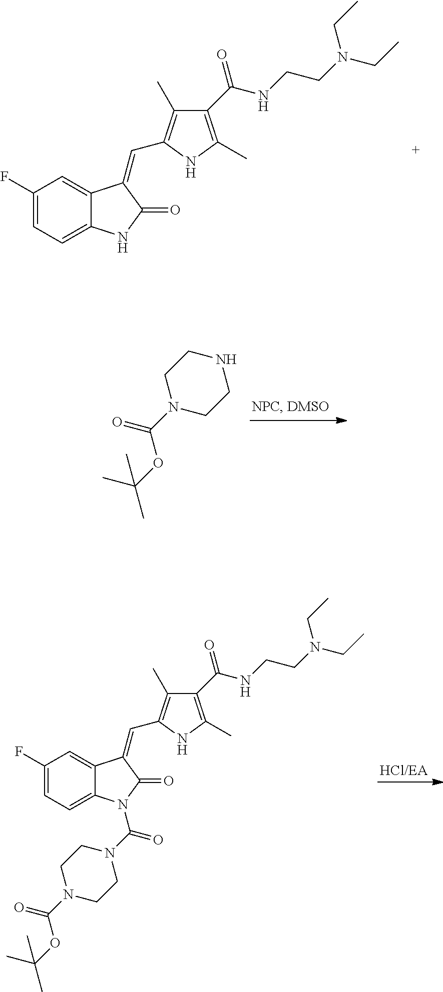

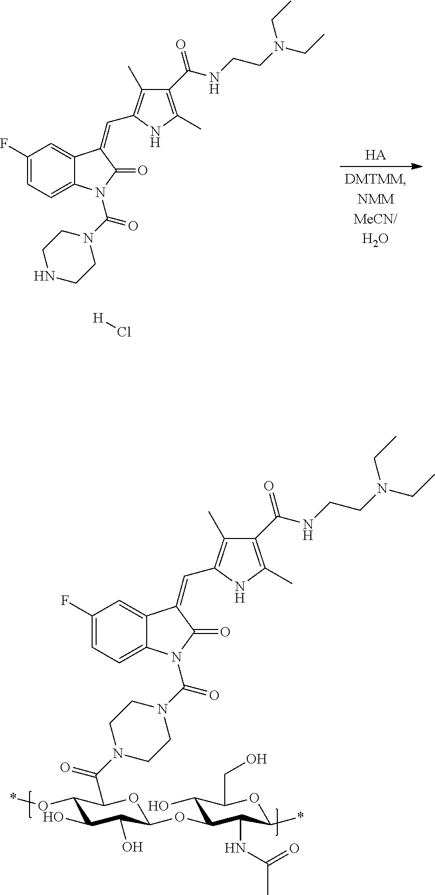

### Step 1: Preparation of tert-butyl (Z)-4-(3-((4-((2-(diethylamino)ethyl)carbamoyl)-3,5-dimethyl-1H-pyrrol-2-yl)methylene)-5-fluoro-2-oxoindoline-1-carbonyl)piperazine-1-carboxylate

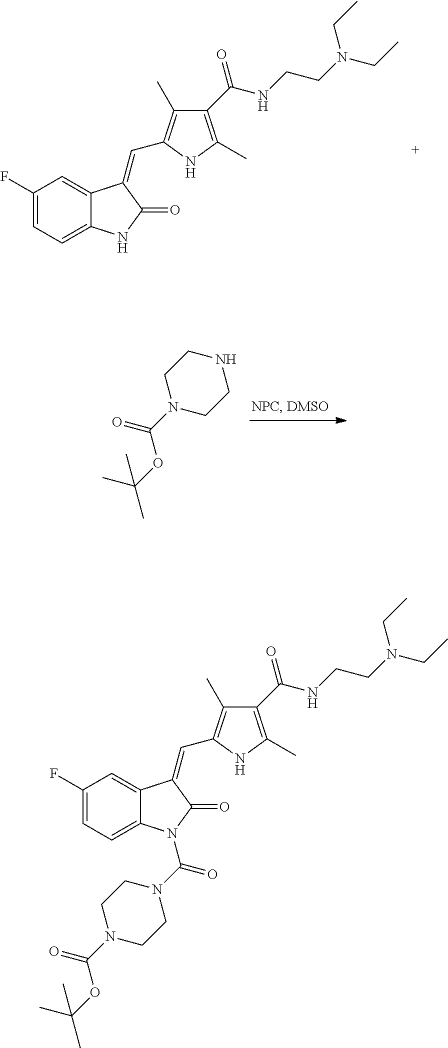

A mixture of (Z)—N-(2-(diethylamino)ethyl)-5-((5-fluoro-2-oxoindolin-3-ylidene)methyl)-2,4-dimethyl-1H-pyrrole-3-carboxamide (1594 mg, 4 mmol) and bis(4-nitrophenyl) carbonate (1460 mg, 4.8 mmol) in DMSO (30 mL) was stirred at room temperature for 7 hours. Then tert-butyl piperazine-1-carboxylate (894 mg, 4.8 mmol) was added and reaction mixture was stirred at 40° C. for 16 hours. The reaction mixture was poured into water and extracted with EtOAc. The organic layer was dried over sodium sulfate, filtered and then concentrated under reduced pressure. The crude residue was purified by column chromatography (DCM:Methanol=40:1) to afford the title compound (1.5 g, Yield: 61.4%). MS (m/z): [M+H]+ calcd for C₃₂H₄₃FN₆O₅, 611.73; found: 611.2. ¹H NMR (400 MHz, Chloroform-d) δ ppm 13.05 (s, 1H), 7.39 (s, 1H), 7.19 (dd, J=8.6, 2.5 Hz, 1H), 7.11 (dd, J=8.7, 4.3 Hz, 1H), 6.90 (td, J=8.9, 2.5 Hz, 1H), 6.66 (s, 1H), 3.89-3.30 (m, 10H), 2.71 (t, J=5.9 Hz, 2H), 2.68-2.57 (m, 7H), 2.51 (s, 3H), 1.49 (s, 9H), 1.07 (t, J=7.1 Hz, 6H).

### Step 2: Preparation of (Z)—N-(2-(diethylamino)ethyl)-5-((5-fluoro-2-oxo-1-(piperazine-1-carbonyl)indolin-3-ylidene)methyl)-2,4-dimethyl-1H-pyrrole-3-carboxamide hydrochloride

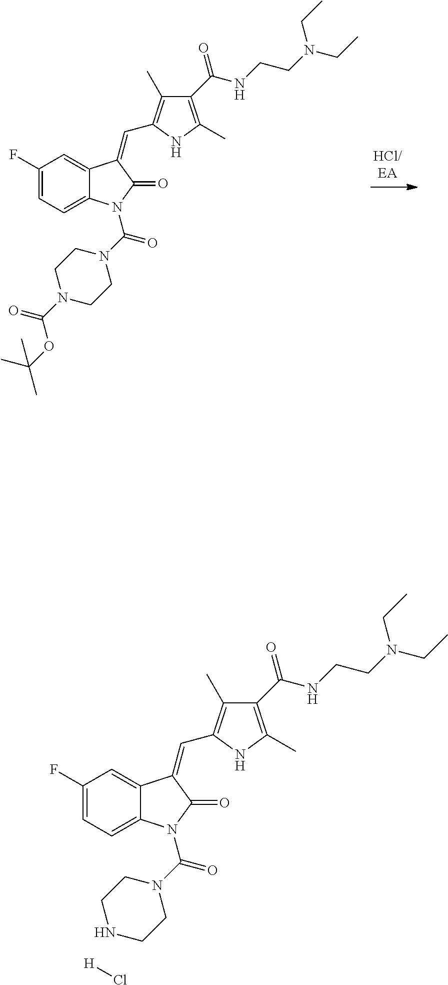

To a solution of tert-butyl (Z)-4-(3-((4-((2-(diethylamino)ethyl)carbamoyl)-3,5-dimethyl-1H-pyrrol-2-yl)methylene)-5-fluoro-2-oxoindoline-1-carbonyl)piperazine-1-carboxylate (1000 mg, 1.64 mmol) in ethyl acetate (50 mL) was added 4M HCl in ethyl acetate solution (8 mL, 32 mmol) dropwisely under N₂ at 0° C. The resulting reaction mixture was stirred at 0° C. for 30 min and then stirred at room temperature for 20 hours. The solution was diluted with ethyl acetate (20 mL) and concentrated under reduced pressure to afford the title compound as a white solid (890 mg, Yield: 99%). MS (m/z): [M+H]+ calcd for C₂₇H₃₅FN₆O₃, 511.61; found: 511.2.

### Step 3: Preparation of conjugate of (Z)—N-(2-(diethylamino)ethyl)-5-((5-fluoro-2-oxo-1-(piperazine-1-carbonyl)indolin-3-ylidene)methyl)-2,4-dimethyl-1H-pyrrole-3-carboxamide and HA

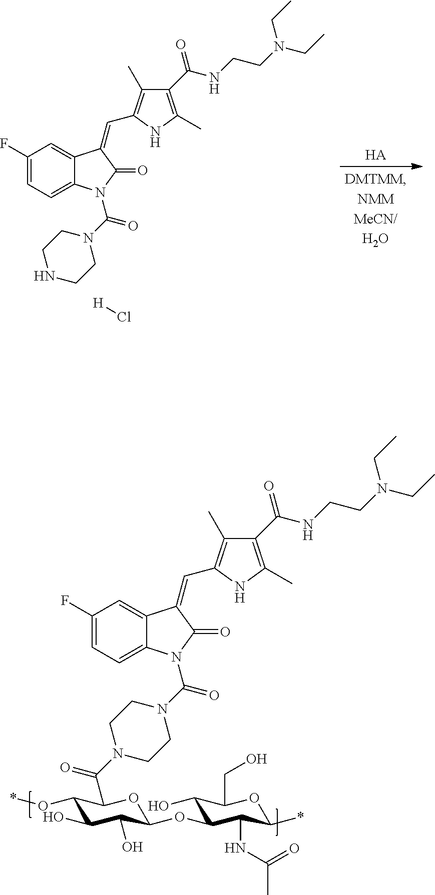

To a solution of sodium hyaluronate (161 mg, 0.4 mmol) in 32 mL of deionized water and 22 mL of acetonitrile while stirring were added (Z)—N-(2-(diethylamino)ethyl)-5-((5-fluoro-2-oxo-1-(piperazine-1-carbonyl)indolin-3-ylidene)methyl)-2,4-dimethyl-1H-pyrrole-3-carboxamidehydrochloride (109 mg, 0.2 mmol) and 4-methylmorpholine (NMM, 20 mg, 0.2 mmol) at room temperature. The reaction mixture was stirred at room temperature for 2 hours and 4-(4,6-dimethoxy-1,3,5-triazin-2-yl)-4-methylmorpholinium chloride (DMTMM, 111 mg, 0.4 mmol) was added and stirred for 72 hours at room temperature. NaCl (234 mg) was then added to the reaction mixture, which was stirred for 1 hour and followed by the dropwise addition of acetone (40 mL) while stirring. The mixture was filtered and the filter cake was washed with acetone, and dried under vacuum to give the title compound as a white solid. (Sodium hyaluronate MW 50 KDa, 0.197 g, Yield: 56.5%, DS: 10%); ¹H NMR (400 MHz, D₂O) δ 7.64-7.46 (m, 0.10H), 7.41-7.21 (m, 0.10H), 7.04-6.71 (m, 0.20H), 4.53-4.27 (m, 2H), 4.06-2.97 (m, 11H), 2.51-1.64 (m, 4.20H), 1.39-1.24 (m, 0.60H).

### Example 99

### Preparation of conjugate of 5-(((Z)-1-((1S,4S)-2,5-diazabicyclo[2.2.1]heptane-2-carbonyl)-5-fluoro-2-oxoindolin-3-ylidene)methyl)-N-(2-(diethylamino)ethyl)-2,4-dimethyl-1H-pyrrole-3-carboxamide and HA

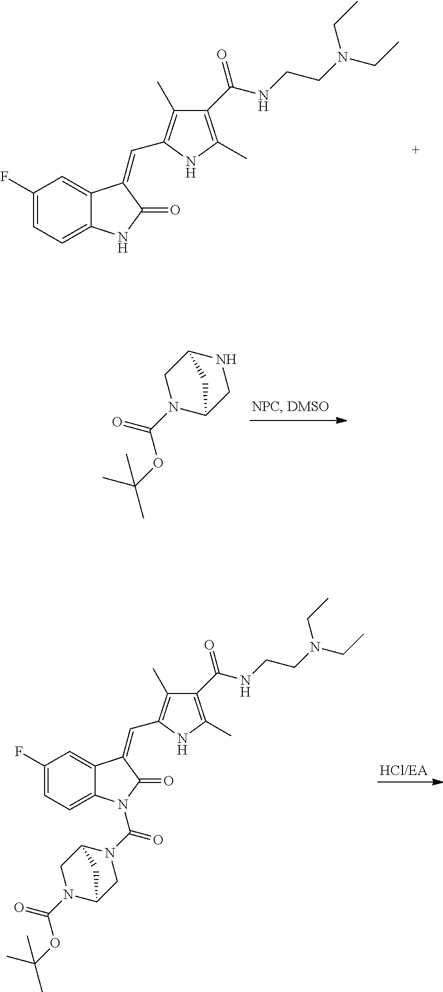

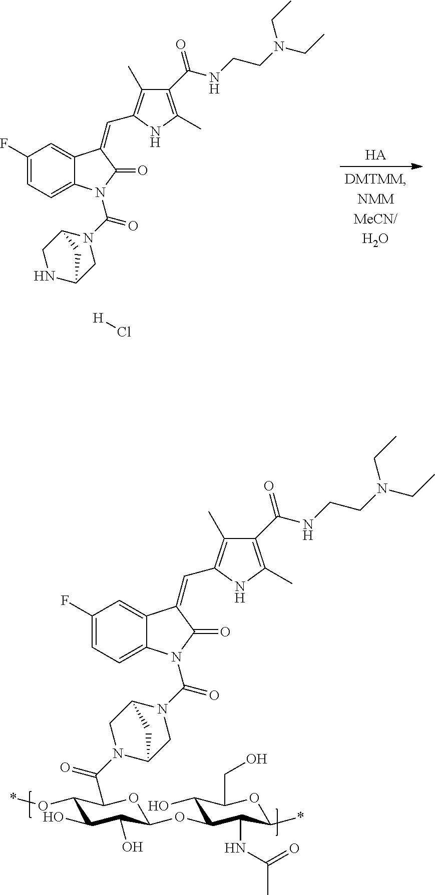

### Step 1: Preparation of tert-butyl (1S,4S)-5-((Z)-3-((4-((2-(diethylamino)ethyl)carbamoyl)-3,5-dimethyl-1H-pyrrol-2-yl)methylene)-5-fluoro-2-oxoindoline-1-carbonyl)-2,5-diazabicyclo[2.2.1]heptane-2-carboxylate

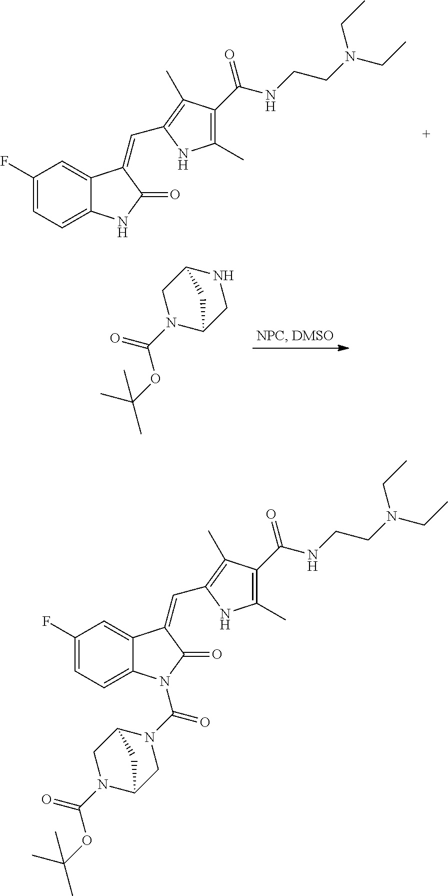

A mixture of (Z)—N-(2-(diethylamino)ethyl)-5-((5-fluoro-2-oxoindolin-3-ylidene)methyl)-2,4-dimethyl-1H-pyrrole-3-carboxamide (1594 mg, 4 mmol) and bis(4-nitrophenyl) carbonate (1460 mg, 4.8 mmol) in DMSO (30 mL) was stirred at room temperature for 7 hours. Then tert-butyl (1S,4S)-2,5-diazabicyclo[2.2.1]heptane-2-carboxylate (992 mg, 5 mmol) was added. The reaction mixture was stirred at 40° C. for 16 hours. The reaction mixture was poured into water and extracted with EtOAc. The organic layer was dried over sodium sulfate, filtered and then concentrated under reduced pressure. The crude residue was purified by column chromatography (DCM:Methanol=8:1) to afford the title compound (1.3 g, Yield: 52%). MS (m/z): [M+H]+ calcd for C₃₃H₄₃FN₆O₅, 623.74; found: 623.2. ¹H NMR (400 MHz, Chloroform-d) δ ppm 13.07 (s, 1H), 7.39 (d, J=9.9 Hz, 1H), 7.18 (h, J=6.6, 5.5 Hz, 2H), 6.91 (td, J=8.6, 2.7 Hz, 1H), 6.57 (s, 1H), 4.98-4.43 (m, 2H), 3.70 (d, J=14.5 Hz, 2H), 3.58-3.34 (m, 4H), 2.68 (t, J=5.9 Hz, 2H), 2.64-2.56 (m, 7H), 2.51 (d, J=3.1 Hz, 3H), 2.03 (dd, J=32.4, 7.3 Hz, 2H), 1.47 (dd, J=20.0, 8.9 Hz, 9H), 1.05 (t, J=7.1 Hz, 6H).

### Step 2: Preparation of 5-(((Z)-1-((1S,4S)-2,5-diazabicyclo[2.2.1]heptane-2-carbonyl)-5-fluoro-2-oxoindolin-3-ylidene)methyl)-N-(2-(diethylamino)ethyl)-2,4-dimethyl-1H-pyrrole-3-carboxamide hydrochloride

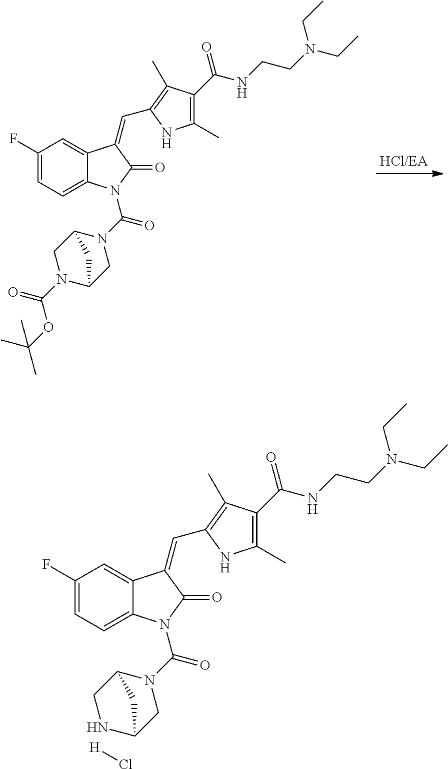

To a solution of tert-butyl (1S,4S)-5-((Z)-3-((4-((2-(diethylamino)ethyl)carbamoyl)-3,5-dimethyl-1H-pyrrol-2-yl)methylene)-5-fluoro-2-oxoindoline-1-carbonyl)-2,5-diazabicyclo[2.2.1]heptane-2-carboxylate (730 mg, 1.17 mmol, 1 eq) in ethyl acetate (15 mL) was added 4M HCl in ethyl acetate solution (2.9 mL, 11.6 mmol) dropwisely under N₂ at 0° C. The resulting reaction mixture was stirred at 0° C. for 30 min and then stirred at room temperature for 20 hours. The solution was diluted with ethyl acetate (20 mL) and concentrated under reduced pressure to afford the title compound as a white solid (0.65 g, Yield: 99%) that was used without further purification. MS (m/z): [M+H]+ calcd for C₂₈H₃₅FN₆O₃, 523.63; found: 522.3.

### Step 3: Preparation of conjugate of 5-(((Z)-1-((1S,4S)-2,5-diazabicyclo[2.2.1]heptane-2-carbonyl)-5-fluoro-2-oxoindolin-3-ylidene)methyl)-N-(2-(diethylamino)ethyl)-2,4-dimethyl-1H-pyrrole-3-carboxamide and HA

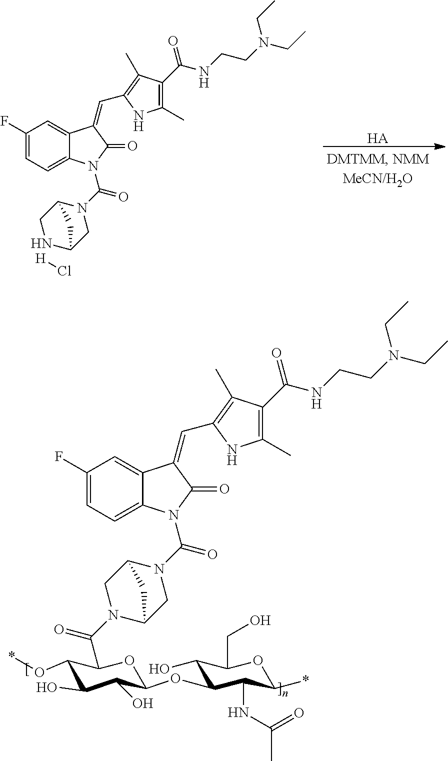

To a solution of sodium hyaluronate (161 mg, 0.4 mmol) in 32 mL of deionized water and 22 mL of acetonitrile were added 5-(((Z)-1-((1S,4S)-2,5-diazabicyclo[2.2.1]heptane-2-carbonyl)-5-fluoro-2-oxoindolin-3-ylidene)methyl)-N-(2-(diethylamino)ethyl)-2,4-dimethyl-1H-pyrrole-3-carboxamidehydrochloride (45 mg, 0.08 mmol) and 4-methylmorpholine (NMM, 8.1 mg, 0.08 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 111 mg, 0.4 mmol) was added and stirred for 72 hours at room temperature. NaCl (234 mg) was then added to the reaction mixture, which was stirred for 1 hour and followed by the dropwise addition of acetone (40 mL) while stirring. The mixture was filtered. The filter cake was washed with acetone, and dried under vacuum to give the title compound as a white solid. (Sodium hyaluronate MW 50 KDa, 0.17 g, Yield: 48.1%, DS: 12%); ¹H NMR (400 MHz, D₂O) δ 7.61-6.69 (m, 0.48H), 4.59-4.29 (m, 2.24H), 4.20-2.93 (m, 10.72H), 2.54-1.58 (m, 4.68H), 1.41-1.23 (m, 0.72H).

### Example 100

### Preparation of conjugate of (Z)—N-(2-(diethylamino)ethyl)-5-((5-fluoro-1-glycyl-2-oxoindolin-3-ylidene)methyl)-2,4-dimethyl-1H-pyrrole-3-carboxamide and HA

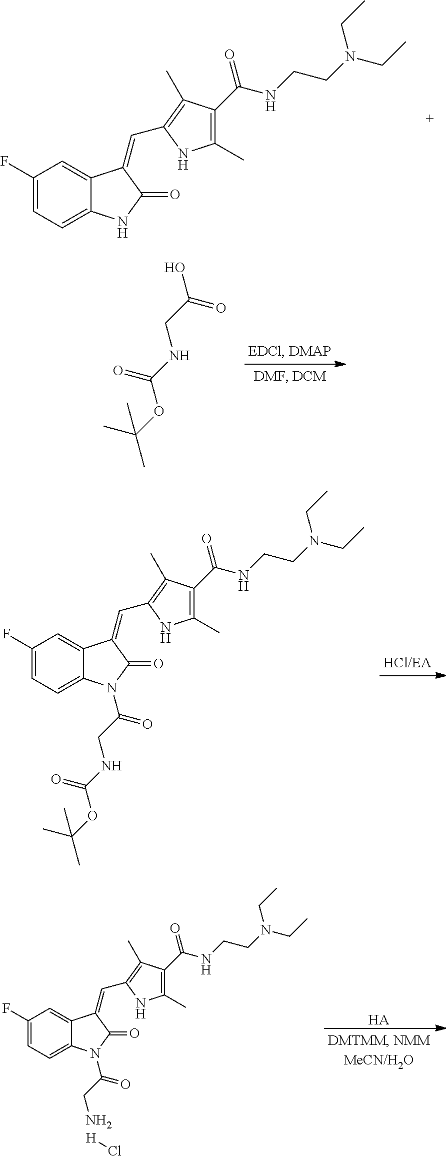

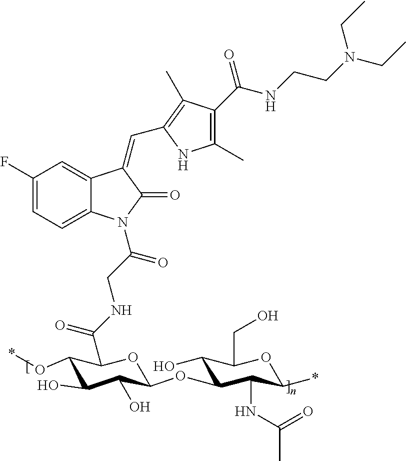

### Step 1: Preparation of tert-butyl (Z)-(2-(3-((4-((2-(diethylamino)ethyl)carbamoyl)-3,5-dimethyl-1H-pyrrol-2-yl)methylene)-5-fluoro-2-oxoindolin-1-yl)-2-oxoethyl)carbamate

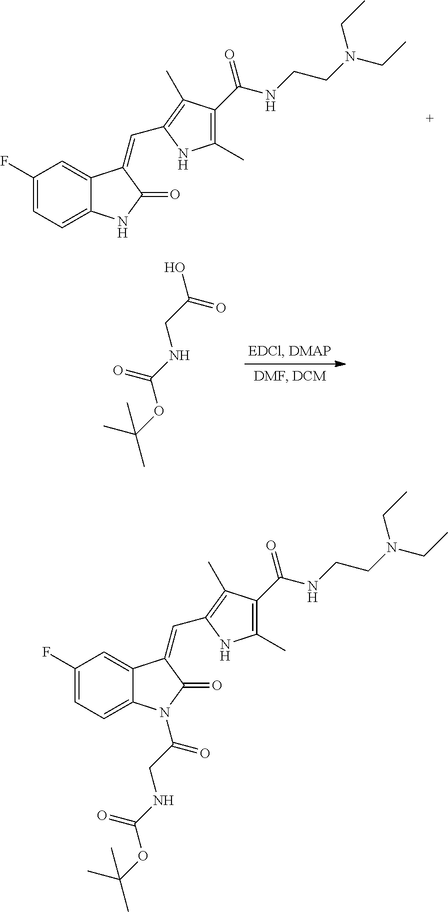

(Z)—N-(2-(diethylamino)ethyl)-5-((5-fluoro-2-oxoindolin-3-ylidene)methyl)-2,4-dimethyl-1H-pyrrole-3-carboxamide (996 mg, 2.5 mmol), (tert-butoxycarbonyl)glycine (876 mg, 5 mmol), 1-ethyl-3-(3-dimethylaminopropyl)carbodiimide (EDCI, 1081 mg, 5.625 mmol), N,N-dimethylpyridin-4-amine (DMAP, 1372 mg, 11.25 mmol) were dissolved in DCM (48 mL) and DMF (12 mL). The reaction mixture was stirred at room temperature for 18 hours. The solution was diluted with DCM and washed with water and saturated brine solution. The organic layer was dried over sodium sulfate, filtered and then concentrated under reduced pressure. The crude residue was purified by column chromatography (DCM/Methanol=50:1-20:1) to afford the title compound (440 mg, Yield: 31.7%). MS (m/z): [M+H]+ calcd for C₂₉H₃₈FN₅O₅, 556.65; found: 556.2. ¹H NMR (400 MHz, Chloroform-d) δ ppm 12.93 (s, 1H), 7.81 (dd, J=8.9, 4.5 Hz, 2H), 7.38 (s, 1H), 7.18 (dd, J=8.4, 2.6 Hz, 1H), 6.97-6.91 (m, 1H), 4.72 (s, 1H), 3.81 (s, 2H), 3.28-3.00 (m, 8H), 2.65 (s, 3H), 2.58 (s, 3H), 1.40 (d, J=28.3 Hz, 15H).

### Step 2: Preparation of (Z)—N-(2-(diethylamino)ethyl)-5-((5-fluoro-1-glycyl-2-oxoindolin-3-ylidene)methyl)-2,4-dimethyl-1H-pyrrole-3-carboxamide hydrochloride

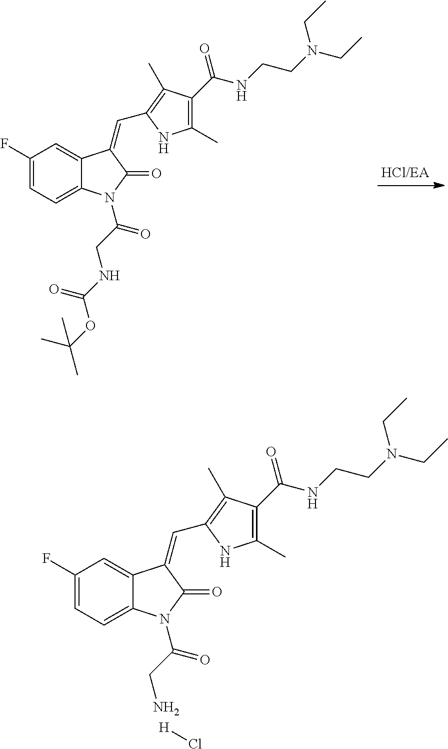

To a solution of tert-butyl (Z)-(2-(3-((4-((2-(diethylamino)ethyl)carbamoyl)-3,5-dimethyl-1H-pyrrol-2-yl)methylene)-5-fluoro-2-oxoindolin-1-yl)-2-oxoethyl)carbamate (222 mg, 0.4 mmol) in ethyl acetate (20 mL) was added 4M HCl in ethyl acetate solution (3 mL, 12 mmol) dropwisely under N₂ at 0° C. The resulting reaction mixture was stirred at 0° C. for 30 min and then stirred at room temperature for 20 hours. The solution was diluted with ethyl acetate (20 mL) and concentrated under reduced pressure to afford the title compound as a white solid (0.14 g, Yield: 71.2%). MS (m/z): [M+H]+ calcd for C₂₄H₃₀FN₅O₃, 456.53; found: 456.2.

### Step 3: Preparation of conjugate of (Z)—N-(2-(diethylamino)ethyl)-5-((5-fluoro-1-glycyl-2-oxoindolin-3-ylidene)methyl)-2,4-dimethyl-1H-pyrrole-3-carboxamide and HA

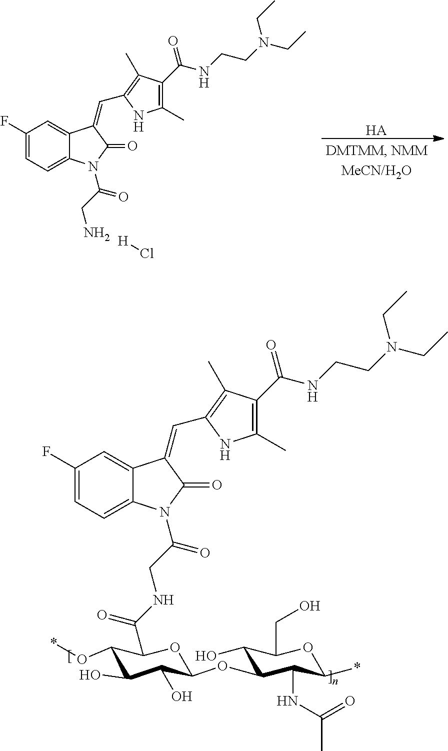

To a solution of sodium hyaluronate (201 mg, 0.5 mmol) in 40 mL of deionized water and 26 mL of acetonitrile were added (Z)—N-(2-(diethylamino)ethyl)-5-((5-fluoro-1-glycyl-2-oxoindolin-3-ylidene)methyl)-2,4-dimethyl-1H-pyrrole-3-carboxamidehydrochloride (56 mg, 0.1 mmol) and 4-methylmorpholine (NMM, 10 mg, 0.1 mmol) at room temperature. The reaction mixture was stirred at this temperature for 2 hours and 4-(4,6-dimethoxy-1,3,5-triazin-2-yl)-4-methylmorpholinium chloride (DMTMM, 28 mg, 0.1 mmol) was added and stirred for 72 hours at room temperature. NaCl (197 mg, 3.37 mmol) was then added to the reaction mixture, which was stirred for 1 hour and followed by the dropwise addition of acetone (40 mL) while stirring. The mixture was filtered and the filter cake was washed with acetone, and dried under vacuum to give the title compound as a white solid. (Sodium hyaluronate M/BW 50 KDa, 0.2 g, Yield: 49%, DS: 9%); ¹H NMR (400 MHz, D₂O) δ ppm 8.03-7.80 (m, 0.09H), 7.51-7.21 (m, 0.18H), 6.99-6.73 (m, 0.09H), 4.43-4.25 (m, 2.18H), 4.11-2.81 (m, 10.90H), 2.51-1.60 (m, 3.54H), 1.40-1.22 (m, 0.54H).

### Example 101

### Preparation of conjugate of 4-aminobutyl (Z)-3-((4-((2-(diethylamino)ethyl)carbamoyl)-3,5-dimethyl-1H-pyrrol-2-yl)methylene)-5-fluoro-2-oxoindoline-1-carboxylate and HA

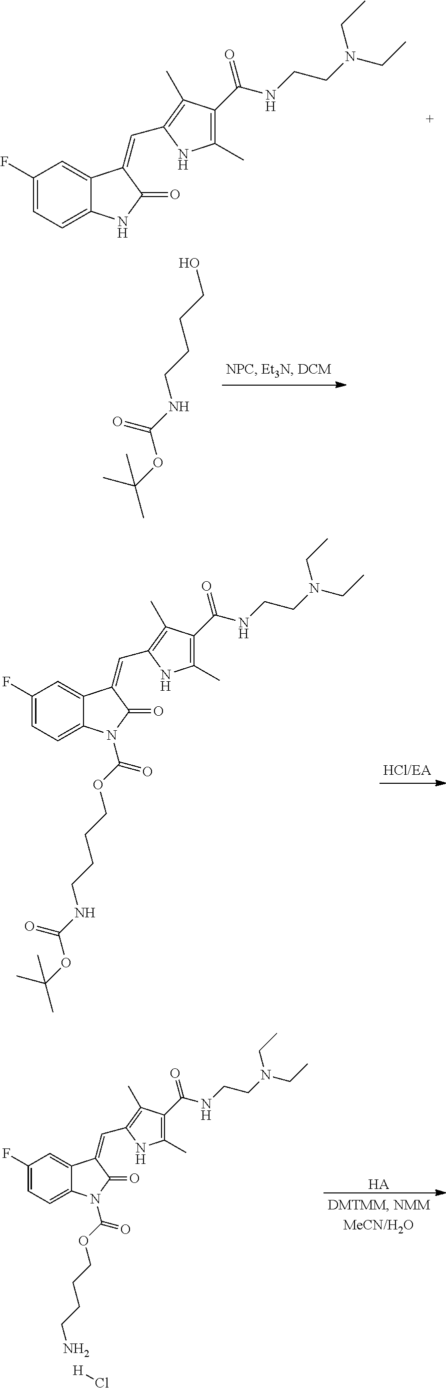

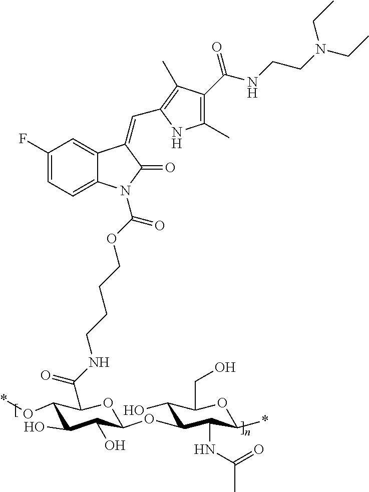

### Step 1: Preparation of 4-((tert-butoxycarbonyl)amino)butyl (Z)-3-((4-((2-(diethylamino)ethyl)carbamoyl)-3,5-dimethyl-1H-pyrrol-2-yl)methylene)-5-fluoro-2-oxoindoline-1-carboxylate

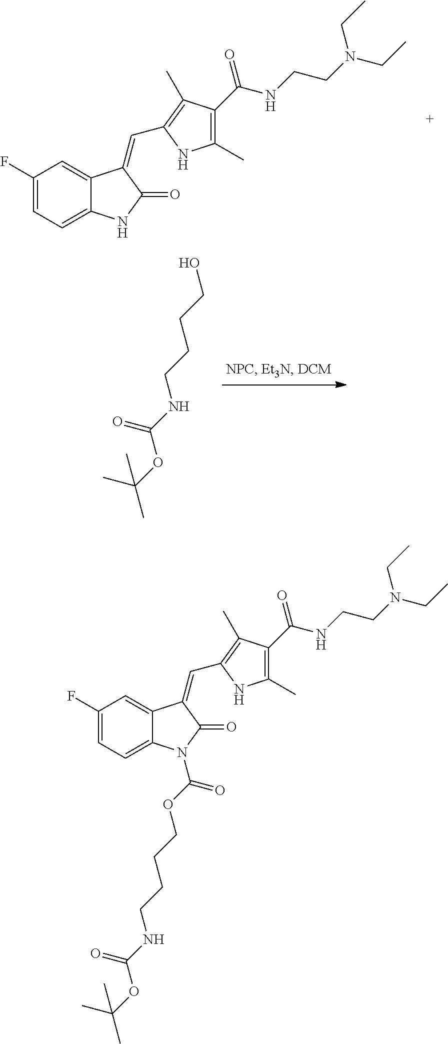

To a stirred mixture of (Z)—N-(2-(diethylamino)ethyl)-5-((5-fluoro-2-oxoindolin-3-ylidene)methyl)-2,4-dimethyl-1H-pyrrole-3-carboxamide (598 mg, 1.5 mmol) and bis(4-nitrophenyl) carbonate (548 mg, 1.8 mmol) in DCM (30 mL) was added triethylamine (379.5 mg, 3.75 mmol). The reaction mixture was heated to 45° C. and stirred at this temperature for 20 hours. Then the reaction mixture was cooled to room temperature. Then tert-butyl (4-hydroxybutyl)carbamate (426 mg, 2.25 mmol) was added. The reaction mixture was stirred at room temperature for 22 hours. The solution was diluted with DCM and washed with saturated NaHCO₃ solution, water and saturated brine solution. The organic layer was dried over sodium sulfate, filtered and then concentrated under reduced pressure. The crude residue was purified by column chromatography (DCM:Methanol=20:1-10:1) to afford the title compound (0.6 g, Yield: 65.2%). MS (m/z): [M+H]+ calcd for C₃₂H₄₄FN₅O₆, 614.73; found: 614.3. ¹H NMR (400 MHz, Chloroform-d) δ 12.93 (s, 1H), 7.81 (dd, J=8.9, 4.5 Hz, 1H), 7.38 (s, 1H), 7.28 (s, 1H), 7.18 (dd, J=8.4, 2.6 Hz, 1H), 6.93 (td, J=9.0, 2.6 Hz, 1H), 4.72 (s, 1H), 4.49 (t, J=6.6 Hz, 2H), 3.81 (s, 2H), 3.27-3.03 (m, 8H), 2.65 (s, 3H), 2.58 (s, 3H), 1.90 (t, J=7.5 Hz, 2H), 1.71 (q, J=7.3 Hz, 2H), 1.44 (s, 9H), 1.38 (d, J=9.3 Hz, 6H).

### Step 2: Preparation of 4-aminobutyl-(Z)-3-((4-((2-(diethylamino)ethyl)carbamoyl)-3,5-dimethyl-1H-pyrrol-2-yl)methylene)-5-fluoro-2-oxoindoline-1-carboxylate hydrochloride

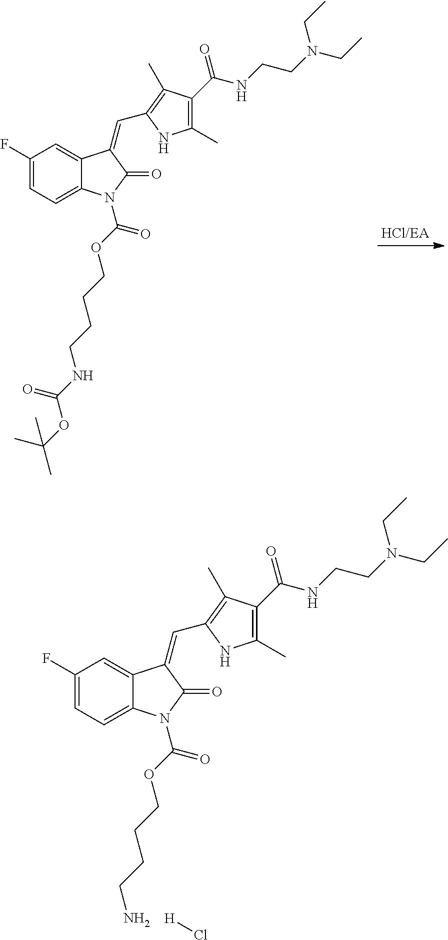 [SMILES: CCN(CC)CCNC(=O)c1c(C)[nH]c(/C=C2\C(=O)N(C(=O)OCCCCNC(=O)OC(C)(C)C)c3ccc(F)cc32)c1C ; CCN(CC)CCNC(=O)c1c(C)[nH]c(/C=C2\C(=O)N(C(=O)OCCCCN)c3ccc(F)cc32)c1C ; Cl]

To a solution of 4-((tert-butoxycarbonyl)amino)butyl-(Z)-3-((4-((2-(diethylamino)ethyl)carbamoyl)-3,5-dimethyl-1H-pyrrol-2-yl)methylene)-5-fluoro-2-oxoindoline-1-carboxylate (184 mg, 0.3 mmol) in ethyl acetate (15 mL) was added 4M HCl in ethyl acetate solution (3 mL, 12 mmol) dropwisely under N₂ at 0° C. The resulting reaction mixture was stirred at 0° C. for 30 min and then stirred at room temperature for 20 hours. The solution was diluted with ethyl acetate (20 mL) and concentrated under reduced pressure to afford the title compound as a white solid (0.12 g, Yield: 72.7%) that was used without further purification. MS (m/z): [M+H]+ calcd for C₂₇H₃₆FN₅O₄, 514.61; found: 514.2.

### Step 3: Preparation of conjugate of 4-aminobutyl (Z)-3-((4-((2-(diethylamino)ethyl)carbamoyl)-3,5-dimethyl-1H-pyrrol-2-yl)methylene)-5-fluoro-2-oxoindoline-1-carboxylate and HA

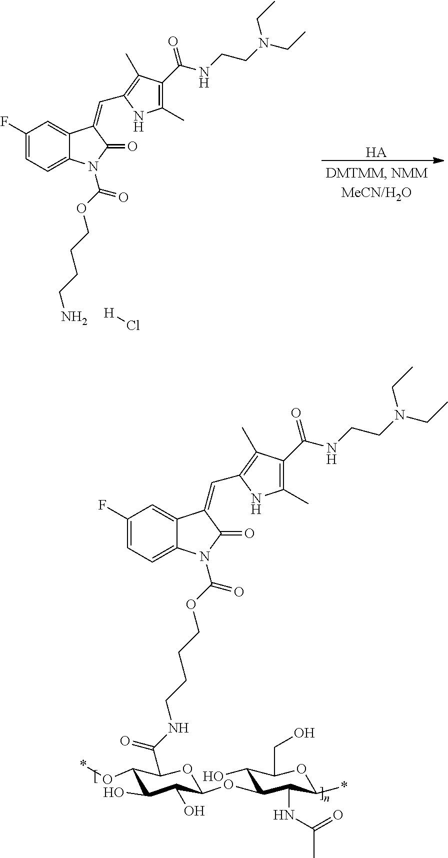

To a solution of Sodium hyaluronate (201 mg, 0.5 mmol) was in 40 mL of deionized water and 26 mL of acetonitrile was added 4-aminobutyl-(Z)-3-((4-((2-(diethylamino)ethyl)carbamoyl)-3,5-dimethyl-1H-pyrrol-2-yl)methylene)-5-fluoro-2-oxoindoline-1-carboxylatehydrochloride (55 mg, 0.1 mmol) and 4-methylmorpholine (NMM, 10 mg, 0.1 mmol) at room temperature. The reaction mixture was stirred at this temperature for 2 hours. To the solution was added 4-(4,6-dimethoxy-1,3,5-triazin-2-yl)-4-methylmorpholinium chloride (DMTMM, 42 mg, 0.15 mmol) was added and stirred for 72 hours at room temperature. NaCl (197 mg, 3.37 mmol) was then added to the reaction mixture, which was stirred for 1 hour and followed by the dropwise addition of acetone (40 mL) while stirring. The mixture was filtered. The filter cake was washed with acetone, and dried under vacuum to give the title compound as a white solid. (Sodium hyaluronate MW 50 KDa, 0.2 g, Yield: 45.7%, DS: 28%); ¹H NMR (400 MHz, D₂O) δ 7.48-6.42 (m, 1.12H), 4.57-4.18 (m, 2.56H), 4.04-2.60 (m, 12.80H), 2.41-1.58 (m, 4.8H), 1.43-1.17 (m, 0.84H).

### Example 102

### Preparation of conjugate of (Z)-5-((1-(4-aminobutanoyl)-5-fluoro-2-oxoindolin-3-ylidene)methyl)-N-(2-(diethylamino)ethyl)-2,4-dimethyl-1H-pyrrole-3-carboxamide and HA

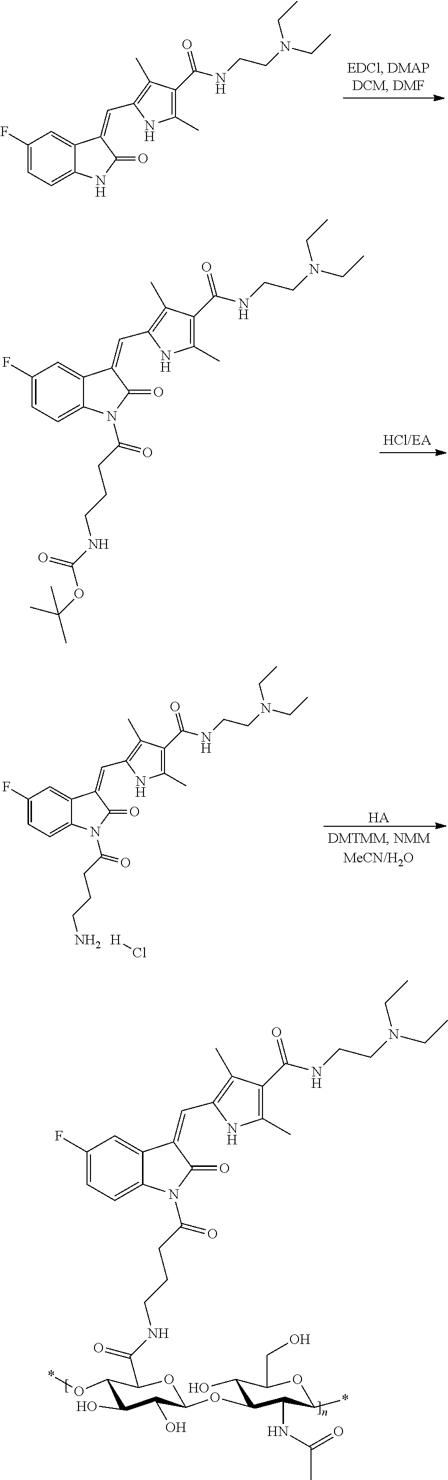

### Step 1: Preparation of tert-butyl (Z)-(4-(3-((4-((2-(diethylamino)ethyl)carbamoyl)-3,5-dimethyl-1H-pyrrol-2-yl)methylene)-5-fluoro-2-oxoindolin-1-yl)-4-oxobutyl)carbamate

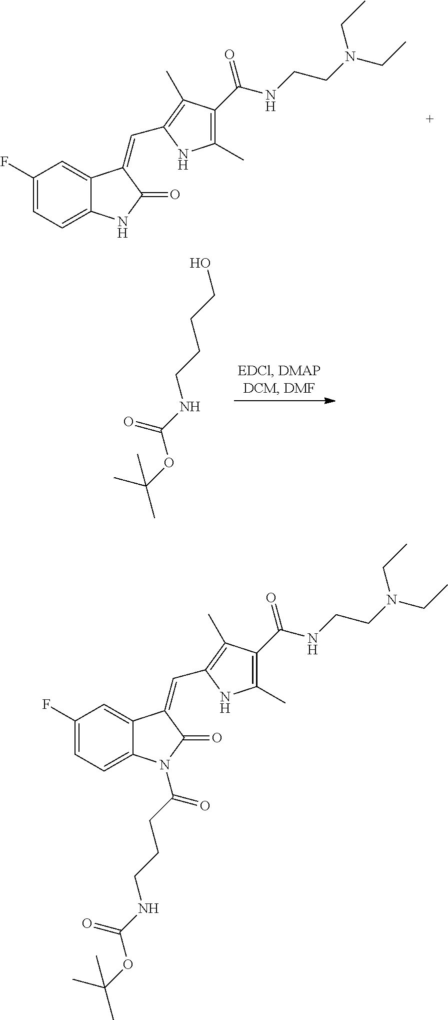

(Z)—N-(2-(diethylamino)ethyl)-5-((5-fluoro-2-oxoindolin-3-ylidene)methyl)-2,4-dimethyl-1H-pyrrole-3-carboxamide (797 mg, 2 mmol), 4-((tert-butoxycarbonyl)amino)butanoic acid (813 mg, 4 mmol), 1-ethyl-3-(3-dimethylaminopropyl)carbodiimide (EDCI, 864.5 mg, 4.5 mmol), N,N-dimethylpyridin-4-amine (DMAP, 1098 mg, 9 mmol) and DIPEA (1034 mg, 8 mmoL) were dissolved in DCM (48 mL) and DMF (12 mL). The reaction mixture was stirred at room temperature for 16 hours. The solution was diluted with DCM and washed with water and saturated brine solution. The organic layer was dried over sodium sulfate, filtered and then concentrated under reduced pressure. The crude residue was purified by column chromatography (DCM/MeOH=50:1-10:1) to afford the title compound (560 mg, Yield: 48%). MS (m/z): [M+H]+ calcd for C₃₁H₄₂FN₅O₅, 584.71; found: 584.2. ¹H NMR (400 MHz, Chloroform-d) δ ppm 12.85 (s, 1H), 8.21 (dd, J=9.0, 4.7 Hz, 1H), 7.98 (s, 1H), 7.37 (s, 1H), 7.16 (dd, J=8.5, 2.7 Hz, 1H), 6.92 (td, J=9.0, 2.6 Hz, 1H), 4.73 (s, 1H), 3.86 (s, 2H), 3.33-3.19 (m, 5H), 3.14 (d, J=7.6 Hz, 3H), 2.69 (s, 3H), 2.58 (s, 3H), 1.98 (q, J=7.1 Hz, 2H), 1.47-1.36 (m, 11H), 1.35-1.18 (m, 6H).

### Step 2: Preparation of (Z)-5-((1-(4-aminobutanoyl)-5-fluoro-2-oxoindolin-3-ylidene)methyl)-N-(2-(diethylamino)ethyl)-2,4-dimethyl-1H-pyrrole-3-carboxamide hydrochloride

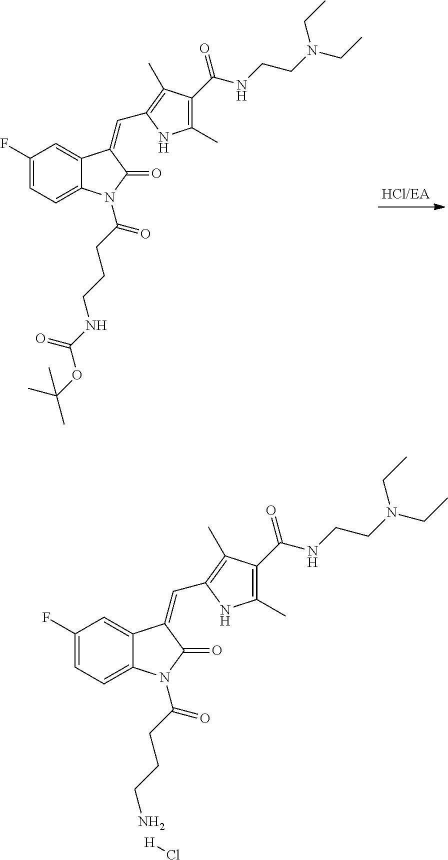

To a solution of tert-butyl (Z)-(4-(3-((4-((2-(diethylamino)ethyl)carbamoyl)-3,5-dimethyl-1H-pyrrol-2-yl)methylene)-5-fluoro-2-oxoindolin-1-yl)-4-oxobutyl)carbamate (204 mg, 0.35 mmol) in ethyl acetate (15 mL) was added 4M HCl in ethyl acetate solution (3 mL, 12 mmol) dropwisely under N₂ at 0° C. The resulting reaction mixture was stirred at 0° C. for 30 min and then stirred at room temperature for 20 hours. The solution was diluted with ethyl acetate (20 mL) and concentrated under reduced pressure to afford the title compound as a white solid (0.16 g, Yield: 94.6%) that was used without further purification. MS (m/z): [M+H]+ calcd for C₂₆H₃₄FN₅O₃, 484.59; found: 484.2.

### Step 3: Preparation of conjugate of (Z)-5-((1-(4-aminobutanoyl)-5-fluoro-2-oxoindolin-3-ylidene)methyl)-N-(2-(diethylamino)ethyl)-2,4-dimethyl-1H-pyrrole-3-carboxamide and HA

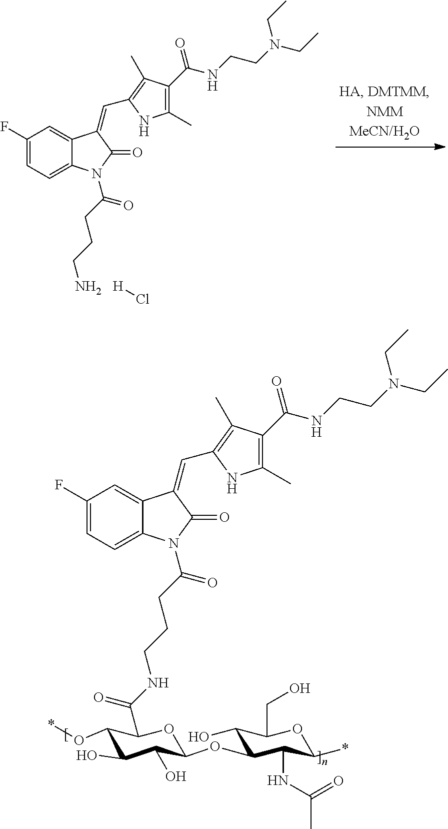

To a solution of sodium hyaluronate (136 mg, 0.337 mmol) was dissolved in 30 mL of deionized water and 19.5 mL of acetonitrile were added (Z)-5-((1-(4-aminobutanoyl)-5-fluoro-2-oxoindolin-3-ylidene)methyl)-N-(2-(diethylamino)ethyl)-2,4-dimethyl-1H-pyrrole-3-carboxamide (150 mg, 0.236 mmol) and 4-methylmorpholine (NMM, 23.9 mg, 0.236 mmol) at room temperature. The reaction mixture was stirred at this temperature for 2 hours. To the solution 4-(4,6-dimethoxy-1,3,5-triazin-2-yl)-4-methylmorpholinium chloride (DMTMM, 93 mg, 0.337 mmol) was added and stirred for 72 hours at room temperature. NaCl (197 mg, 3.37 mmol) was then added to the reaction mixture, which was stirred for 1 hour and followed by the dropwise addition of acetone (200 mL). The mixture was filtered and the filter cake was washed with acetone, and dried under vacuum to give the title compound as a white solid. (153 mg, yield: 53%, DS=10%). ¹H NMR (400 MHz, D₂O) δ ppm 8.03-7.89 (m, 0.1H), 7.47-7.25 (m, 0.2H), 7.02-6.87 (m, 0.1H), 4.58-4.17 (m, 2H), 4.12-2.92 (m, 11H), 2.56-2.45 (m, 0.6H), 2.12-1.70 (m, 3.2H), 1.58-1.46 (m, 0.2H), 1.42-1.25 (m, 0.6H).

### Example 103

### Drug Release of the Drug Delivery System

### Method

The drug release and stability experiments were run using the test compounds. About 2.5±1.0 mg/mL (with regard to conjugate) solution of each test compound was prepared with 10 mM PBS buffer (pH=7.4) in Millipore Amicon® Ultra-0.5 ml 30 k Ultrafiltration centrifuge tube. The solution was kept swelling for 1 h at room temperature and then placed in a shaker at 100 rpm, 37° C. for continuous experiment. At the same time point of each day, samples were centrifuged at 10000 rpm for 1 h. The aliquot was transferred to HPLC vial for analysis. Add 0.4 mL of 10 mM PBS buffer into the centrifuge tube and continue the experiment.

For the HPLC analysis at each time point, the peak areas of all relevant peaks from the chromatograms were retrieved and the concentration of free drug was calculated. Average release of free drug was calculated based on the sum of free drug and numbers of experiment days. The computing equation is as below.

$$Average release of free drug ( µg / mL / d ) = \frac{the sum of free drug ( µg / mL )}{numbers of experiment days ( d )}$$

The sample degradation rate was calculated based on the concentration and degree of substitution (NMR) for conjugated drug with regard to the initial starting point of the experiment (at t=0). The computing equation is as below.

$$Release rate of conjugate ( \% / d ) = \frac{Average release of free drug ( µg / mL / d )}{Concentration ( mg / mL ) * Degree of substitution ( NMR ) * 1000}$$

### Results

Results of the drug release for exemplary drug delivery systems of the present disclosure are shown in Table 1.

TABLE 1

| Example No. | Release rate (%/day) |
|---|---|
| Example 2 | 0.26% |
| Example 3 | 0.26% |
| Example 6 | 1.62% |
| Example 9 | 0.09% |
| Example 10 | 0.32% |
| Example 11 | 0.75% |
| Example 13 | 0.75% |
| Example 17 | 0.14% |
| Example 21 | 0.07% |
| Example 23 | 1.01% |
| Example 24 | 1.39% |
| Example 26 | 0.22% |
| Example 27 | 4.69% |
| Example 28 | 2.02% |
| Example 29 | 5.05% |
| Example 30 | 6.00% |
| Example 31 | 18.37% |
| Example 33 | 6.00% |
| Example 34 | 3.85% |
| Example 35 | 1.78% |
| Example 36 | 2.60% |
| Example 39 | 0.02% |
| Example 41 | 0.43% |
| Example 42 | 0.01% |
| Example 43 | 0.06% |
| Example 48 | 0.07% |
| Example 52 | 1.42% |
| Example 53 | 11.18% |
| Example 54 | 22.08% |
| Example 57 | 9.28% |
| Example 59 | 0.51% |
| Example 61 | 0.18% |
| Example 68 | 10.81% |
| Example 69 | 16.47% |
| Example 73 | 1.90% |
| Example 74 | 4.81% |
| Example75 | 7.14% |
| Example 76 | 0.03% |
| Example 77 | 0.13% |
| Example 78 | 0.08% |
| Example 79 | 1.21% |
| Example 80 | 0.02% |
| Example 81 | 0.28% |
| Example 82 | 0.07% |
| Example 83 | <0.01% |
| Example 84 | 0.42% |
| Example 85 | 7.66% |
| Example 86 | 0.62% |
| Example 87 | 0.93% |
| Example 88 | 0.05% |
| Example 89 | <0.01% |
| Example 90 | <0.01% |
| Example 91 | <0.01% |
| Example 92 | 0.62% |
| Example 93 | 0.35% |
| Example 94 | 1.03% |
| Example 95 | 60.56% |
| Example 96 | 0.15% |
| Example 97 | 3.4% |
| Example 98 | <0.01% |
| Example 99 | <0.01% |
| Example 100 | 8.53% |
| Example 101 | 0.42% |
| Example 102 | 13.6% |

The foregoing description is considered as illustrative only of the principles of the present disclosure. Further, since numerous modifications and changes will be readily apparent to those skilled in the art, it is not desired to limit the invention to the exact construction and process shown as described above. Accordingly, all suitable modifications and equivalents may be considered to fall within the scope of the invention as defined by the claims that follow.

1. A drug delivery system for locally delivering a therapeutic agent at a controlled rate, the drug delivery system comprising:
a biopolymer comprising at least a first binding group BG1 selected from the group consisting of hydroxyl group, carboxylic group, amino group, and a combination thereof;
a therapeutic agent comprising at least a second binding group BG2 selected from the group consisting of hydroxyl group, carboxylic group, amino group, amide group, amine group and a combination thereof; and
a linker covalently linking the biopolymer to the therapeutic agent and capable of retaining the therapeutic agent in the location of administration;
wherein the linker comprises a structure of formula (I): 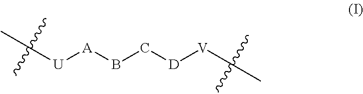
wherein
U is connected to the biopolymer through BG1 such that at least one linkage selected from ester or amide is formed, and U is selected from the group consisting of a direct bond, —N(R¹)—, —O—, —C(═O)— and 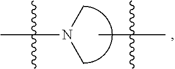
wherein 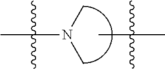
is a nitrogen-containing heterocyclyl optionally comprising one or more additional heteroatoms selected from N, O or S;
A is selected from a direct bond, alkyl and —(CH₂CH₂O)ₘ—, wherein said alkyl is optionally substituted with one or more R^{a} groups;
B is selected from the group consisting of a direct bond, alkyl, cycloalkyl, heterocyclyl, aryl, heteroaryl, —O-cycloalkyl, —O-heterocyclyl, —O-aryl, —O-heteroaryl, wherein each of alkyl, cycloalkyl, heterocyclyl, aryl and heteroaryl is optionally substituted with one or more R^{b} groups;
C is selected from a direct bond, —C(═O)—, —C(═O)N(R²)—, —N(R²)C(═O)—, —[CH₂NHC(═O)]ₙ—, —[NHC(═O)CH₂]ₙ—, and —NH(CH₂)ₚC(═O)—;
D is selected from a direct bond, alkyl, and aryl, wherein said alkyl is optionally substituted with one or more R^{c} groups;
V is connected to the therapeutic agent through BG2 such that at least one linkage selected from the group consisting of amide, urea, thiourea, carbamate, thiocarbamate, phosphoramidate, aza-acetal and combination thereof is formed, and V is selected from the group consisting of a direct bond, —C(═O)—, —N(R²)C(═O)—, N(R²)C(S)—, —OC(═O)—, —OC(═S)—, —OC(═O)OCH₂—, —C(═O)OCH₂—, —N(R²)C(═O)OCH₂—, —OP(═O)(OPh)-, and —N(R²)P(═O)(OPh)-;
R¹ and R² are independently selected from the group consisting of hydrogen, alkyl, alkenyl and alkynyl;
R^{a}, R^{b}, and R^{c} are independently selected from halogen, hydroxyl, amino, cyano, nitro, alkyl, alkoxyl, —C(═O)OR^{e}, and ═NH;
R^{e} is an alkyl;
m is an integer from 0 to 4;
n is an integer from 1 to 4; and
p is an integer from 1 to 4.

2-62. (canceled)

63. The drug delivery system of claim 1, wherein,
(i) BG1 is carboxylic group and U is —N(R¹)—, such that an amide linkage is formed;
(ii) BG1 is carboxylic group and U is 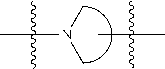
such that an amide linkage is formed;
(iii) BG1 is carboxylic group and U is —O— or a direct bond, such that an ester linkage is formed;
(iv) BG1 is hydroxyl group and U is —C(═O)—, such that an ester linkage is formed; or
(v) BG1 is amino group and U is —C(═O)—, such that an amide linkage is formed.

64. The drug delivery system of claim 63, wherein U is 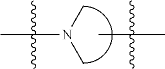
selected from the group consisting of:

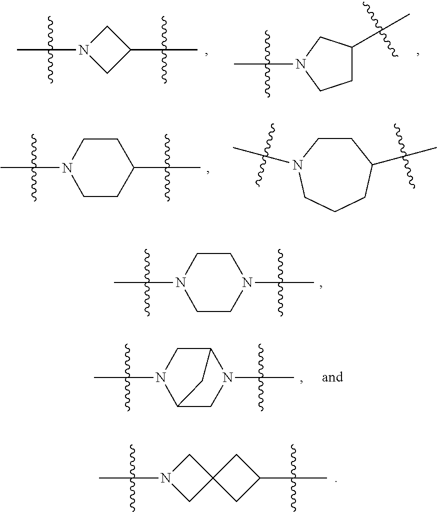

65. The drug delivery system of claim 1, wherein
(i) BG2 is amine group, V is selected from one of the following:
(a) a direct bond;
(b) —N(R²)C(═O)— which is connected to the therapeutic agent through BG2 such that a urea linkage is formed;
(c) —N(R²)C(S)— which is connected to the therapeutic agent through BG2 such that a thiourea linkage is formed;
(d) —OC(═O)— which is connected to the therapeutic agent through BG2 such that a carbamate linkage is formed;
(e) —OC(═S)— which is connected to the therapeutic agent through BG2 such that a thiocarbamate linkage is formed;
(f) —OC(═O)OCH₂— which is connected to the therapeutic agent through BG2 such that an aza-acetal linkage is formed;
(g) —C(═O)OCH₂— which is connected to the therapeutic agent through BG2 such that an aza-acetal linkage is formed;
(h) —N(R²)C(═O)OCH₂— which is connected to the therapeutic agent through BG2 such that an aza-acetal linkage is formed;
(i) —OP(═O)(OPh)- which is connected to the therapeutic agent through BG2 such that a phosphoramidate linkage is formed;
(j) —N(R²)P(═O)(OPh)- which is connected to the therapeutic agent through BG2 such that a phosphoramidate linkage is formed;
(k) —C(═O)— which is connected to the therapeutic agent through BG2 such that an amide linkage is formed;
(ii) BG2 is carboxylic group and V is —O— or a direct bond, such that an ester linkage is formed; or
(iii) BG2 is hydroxyl group and V is —C(═O)—, such that an ester linkage is formed.

66. The drug delivery system of claim 1, wherein:
(i) A is a direct bond, B is selected from the group consisting of a direct bond, cycloalkyl, heterocyclyl, aryl, and heteroaryl;
(ii) A is alkyl, B is selected from the group consisting of a direct bond, cycloalkyl, heterocyclyl, aryl, heteroaryl, —O-cycloalkyl, —O-heterocyclyl, —O-aryl, and —O— heteroaryl; or
(iii) A is —(CH₂CH₂O)ₘ—, B is selected from the group consisting of a direct bond, alkyl, cycloalkyl, heterocyclyl, aryl, and heteroaryl.

67. The drug delivery system of claim 1, wherein:
(i) A is an alkyl, B is selected from the group consisting of a direct bond, cycloalkyl, heterocyclyl, aryl, heteroaryl, —O-cycloalkyl, —O-heterocyclyl, —O-aryl, and —O-heteroaryl, and C is selected from the group consisting of a direct bond, —C(═O)—, —N(R²)C(═O)—, —[CH₂NHC(═O)]ₙ—, —[NHC(═O)CH₂]ₙ—, and —NH(CH₂)ₚC(═O)—; or
(ii) A is —(CH₂CH₂O)ₘ—, B is selected from the group consisting of a direct bond, alkyl, cycloalkyl, heterocyclyl, aryl, and heteroaryl, and C is a direct bond or —N(R²)C(═O)—.

68. The drug delivery system of claim 1, wherein the linker comprises a structure of any one of formula (Ia) to (Im): 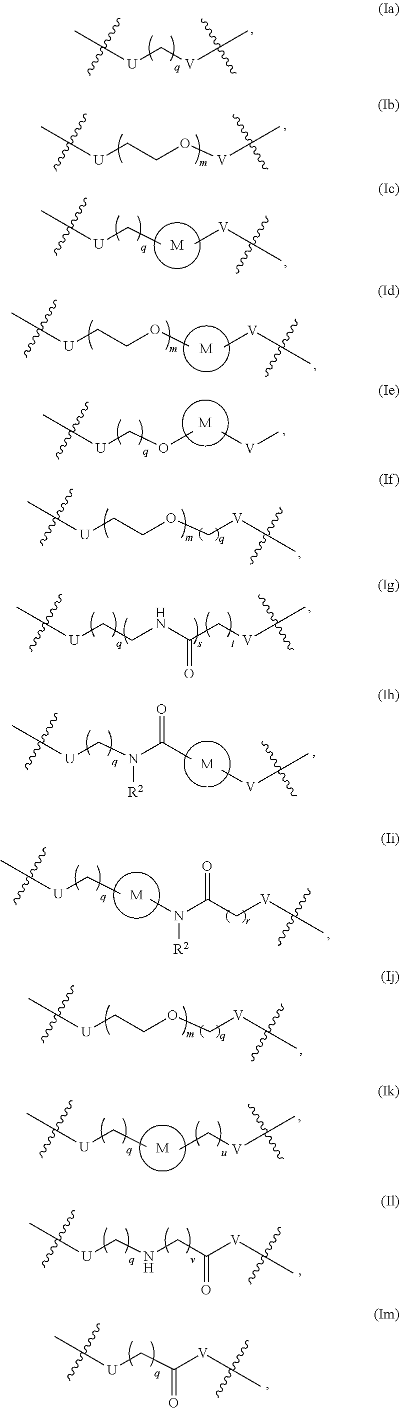 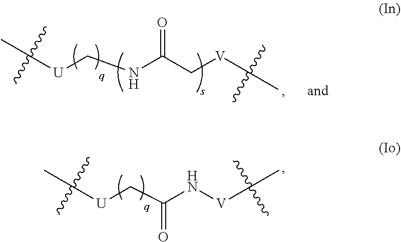
wherein,
U and V are as defined in claim 1;
M is selected from the group consisting of cycloalkyl, heterocyclyl, aryl, and heteroaryl, each of which is optionally substituted with one or more R^{b} groups;
each of 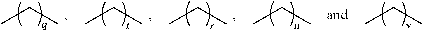
is optionally substituted with —C(═O)OCH₃; and
q, r, s, t, u and v are independently integer from 0 to 5.

69. The drug delivery system of claim 68, wherein M is selected from the group consisting of cyclohexyl, phenyl, pyridinyl, thiazolyl, and adamantyl.

70. The drug delivery system of claim 68, wherein the linker comprises a structure selected from the group consisting of: 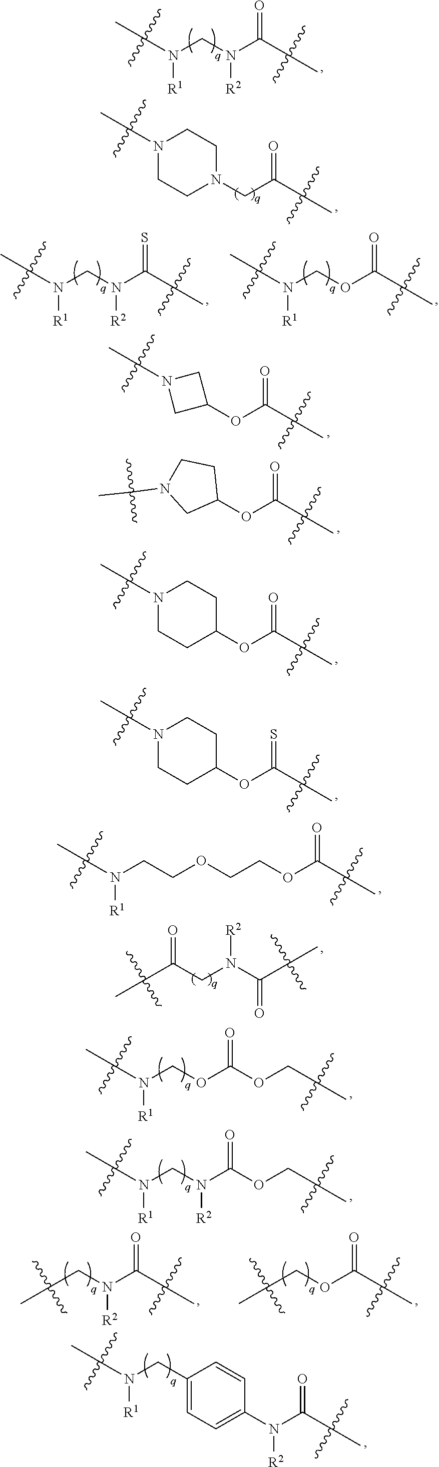 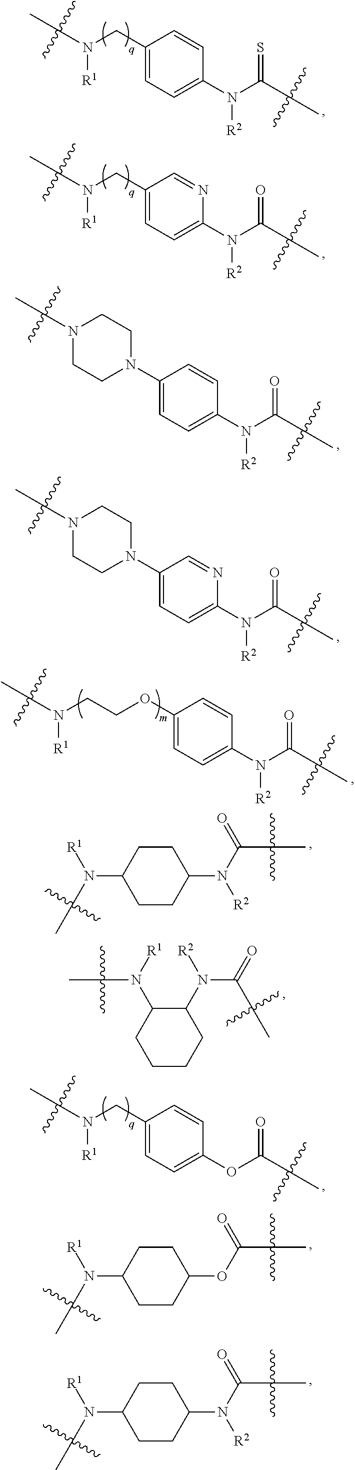 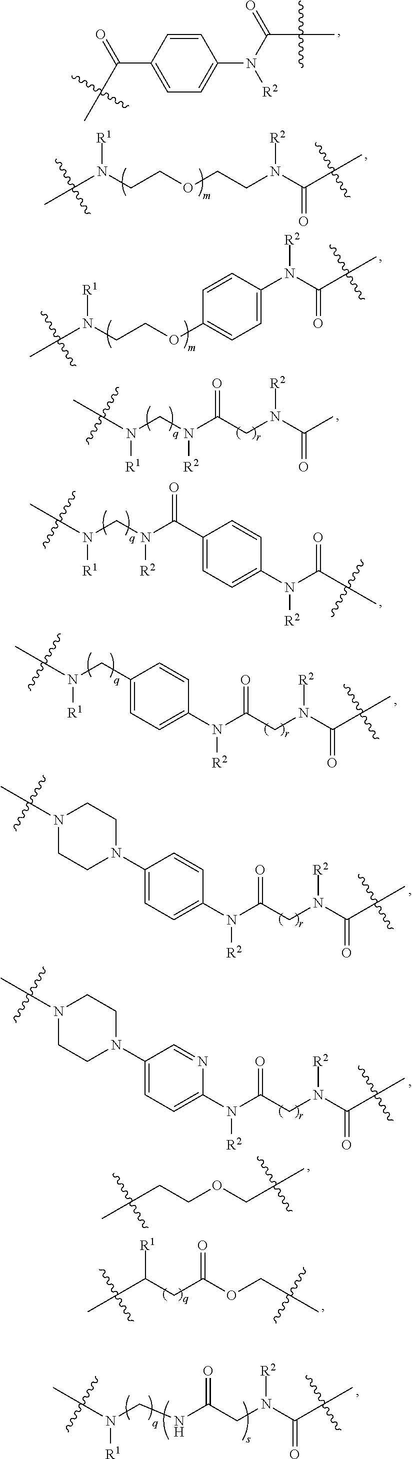 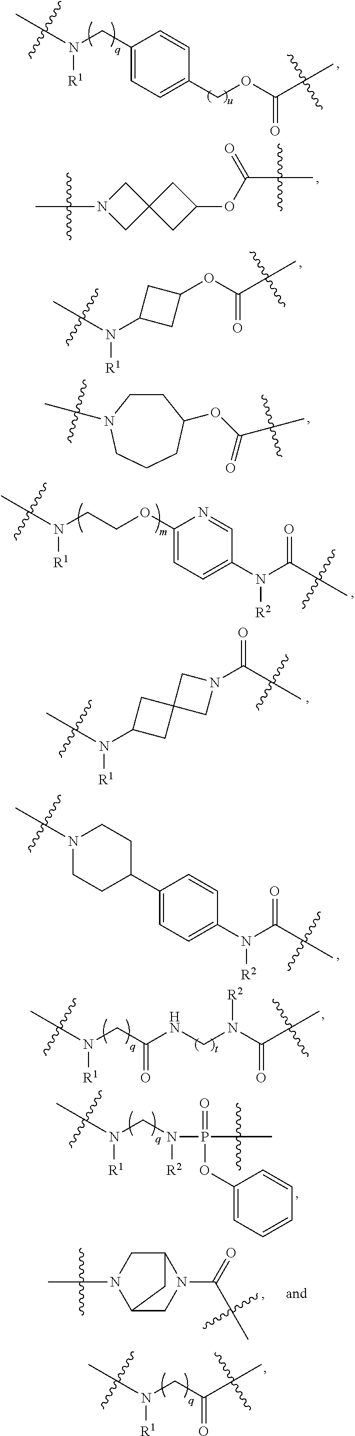
wherein each of 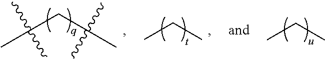
is optionally substituted with C(═O)OCH₃.

71. The drug delivery system of claim 1, wherein the biopolymer is selected from the group consisting of hyaluronic acid, chitosan, chitin, chondroitin, or derivatives thereof.

72. The drug delivery system of claim 71, wherein the biopolymer is hyaluronic acid.

73. The drug delivery system of claim 71, wherein the biopolymer is chondroitin.

74. The drug delivery system of claim 1, wherein the therapeutic agent is selected from the group consisting of anti-cancer drugs, nonsteroidal anti-inflammatory drugs (NSAIDs), Janus kinase (JAK) inhibitors, and vascular endothelial growth factor (VEGF) inhibitors, wherein the NSAID is selected from the group consisting of Piroxicam, Meloxicam, and Diclofenac, the JAK inhibitor is selected from the group consisting of Tofacitinib, Ruxolitinib, Baricitinib, Peficitinib, Fedratinib, Oclacitinib and Upadacitinib, the VEGF inhibitor is selected from the group consisting of Axitinib, Lapatinib, Lenvatinib, Pazopanib, Nintedanib, Sunitinib, and Vandetanib.

75. The drug delivery system of claim 74, wherein the therapeutic agent is Tofacitinib, Upadacitinib, Ruxolitinib, Baricitinib, or Oclacitinib.

76. The drug delivery system of claim 74, wherein the therapeutic agent is Nintedanib or Sunitinib.

77. The drug delivery system of claim 75, wherein the therapeutic agent is Tofacitinib.

78. The drug delivery system of claim 1 selected from the group consisting of:

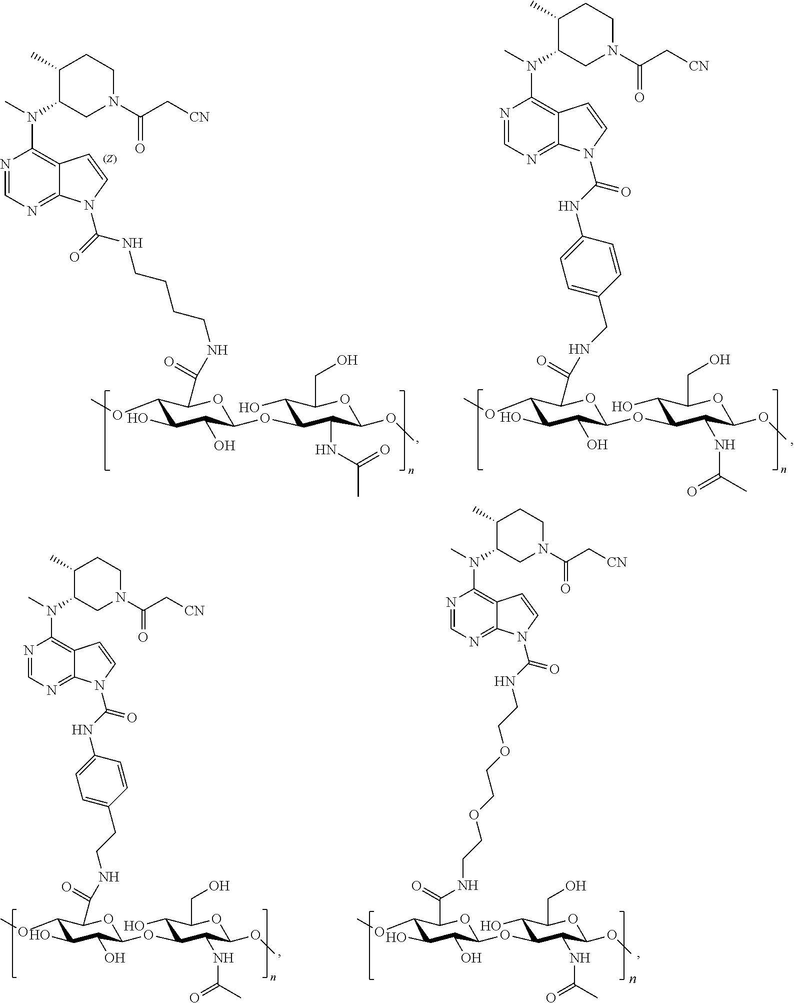

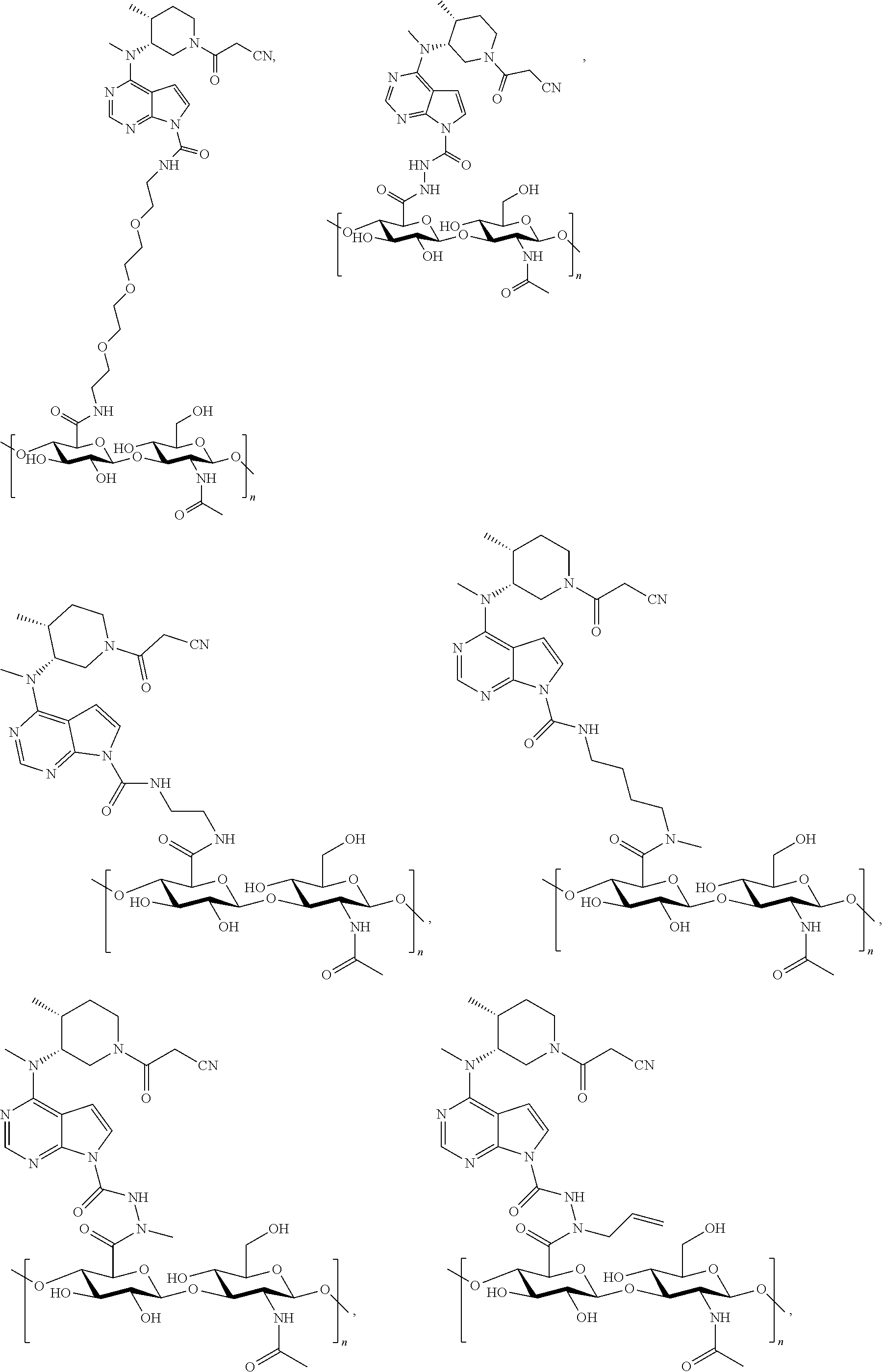

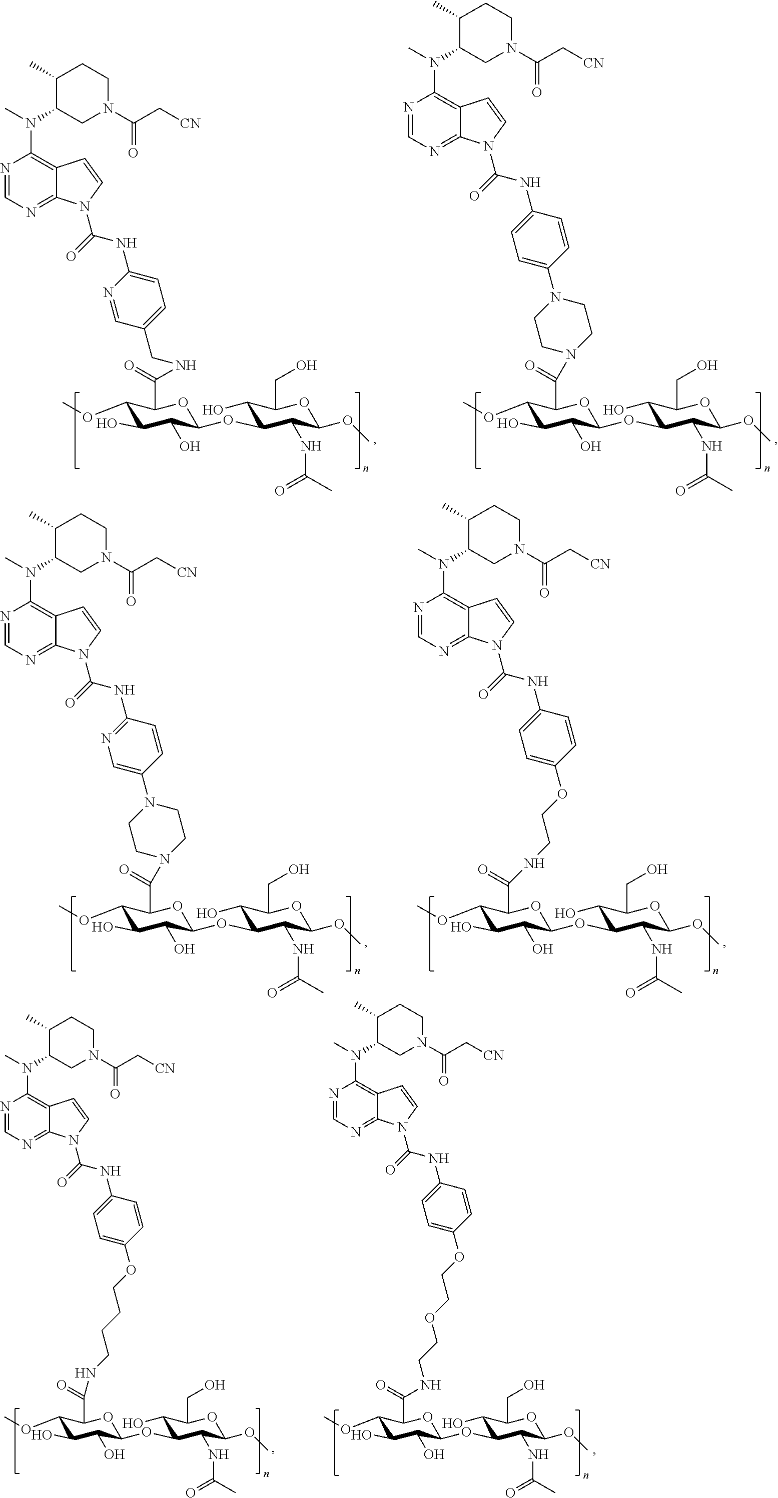

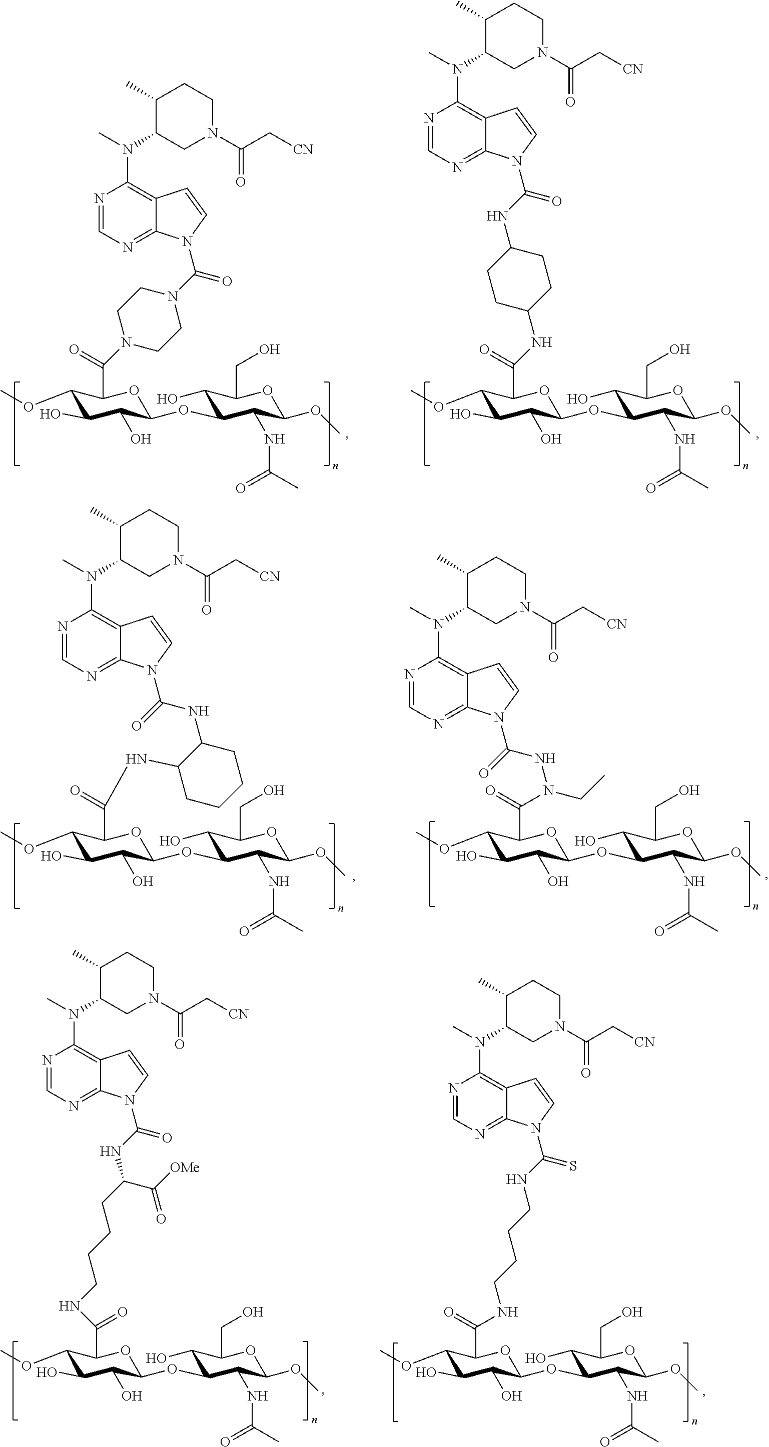

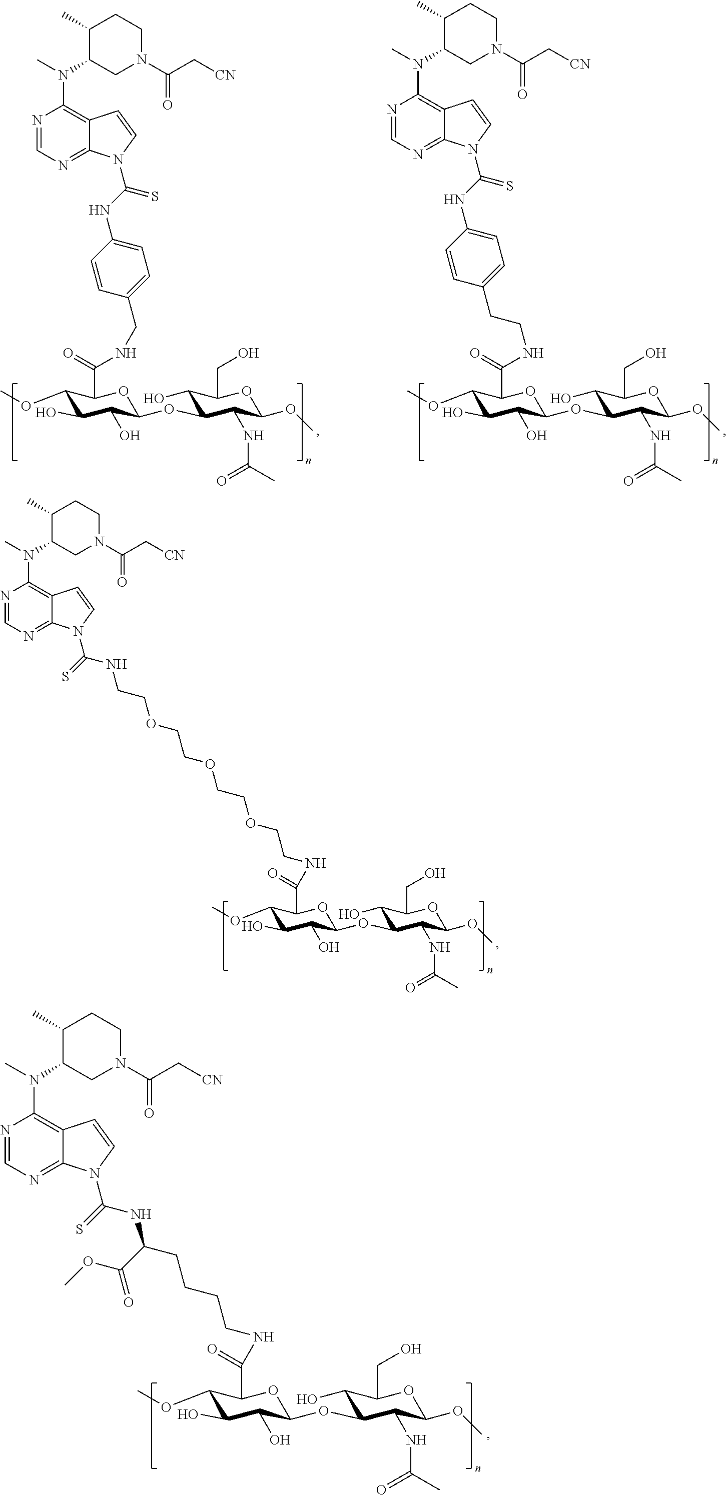

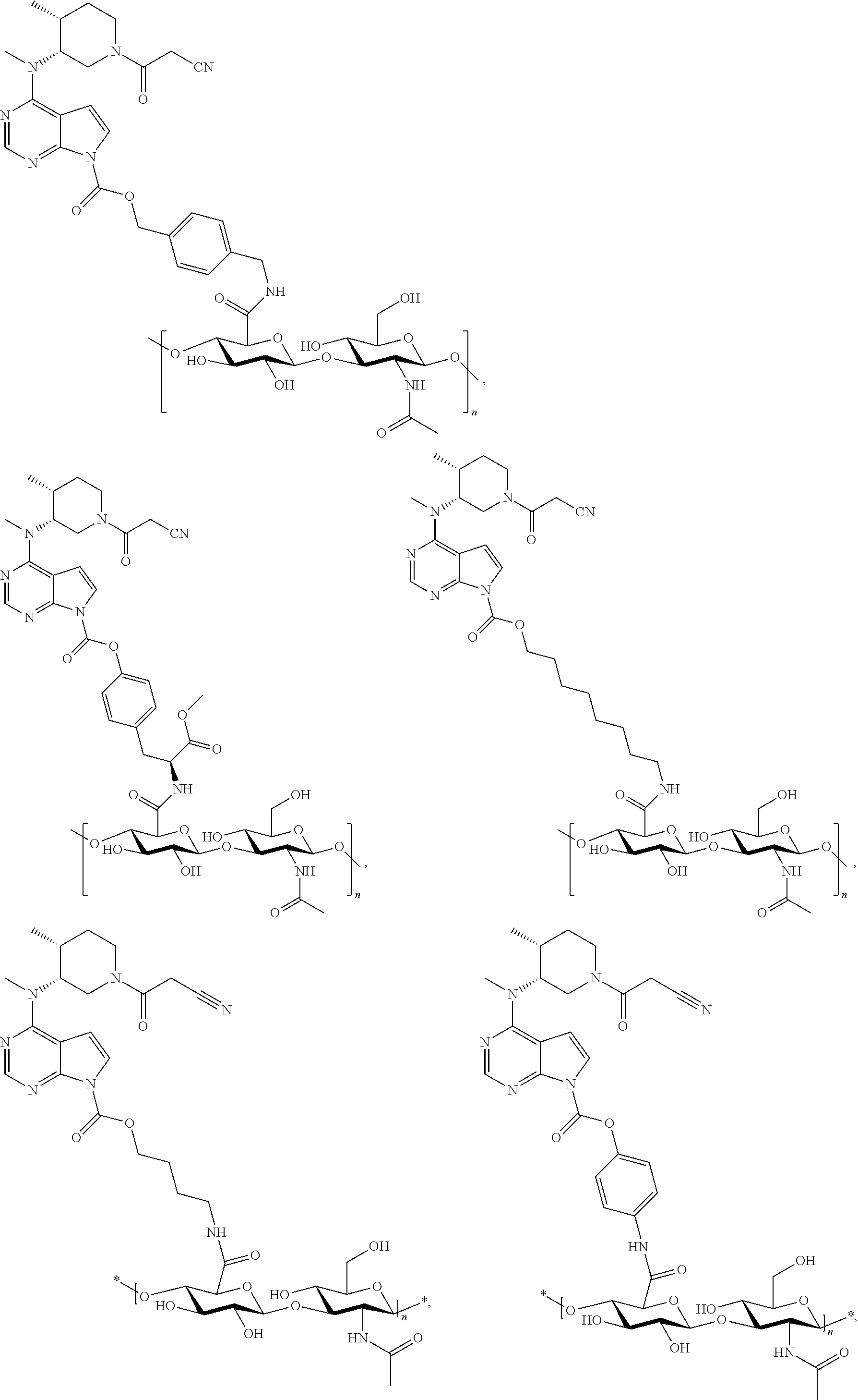

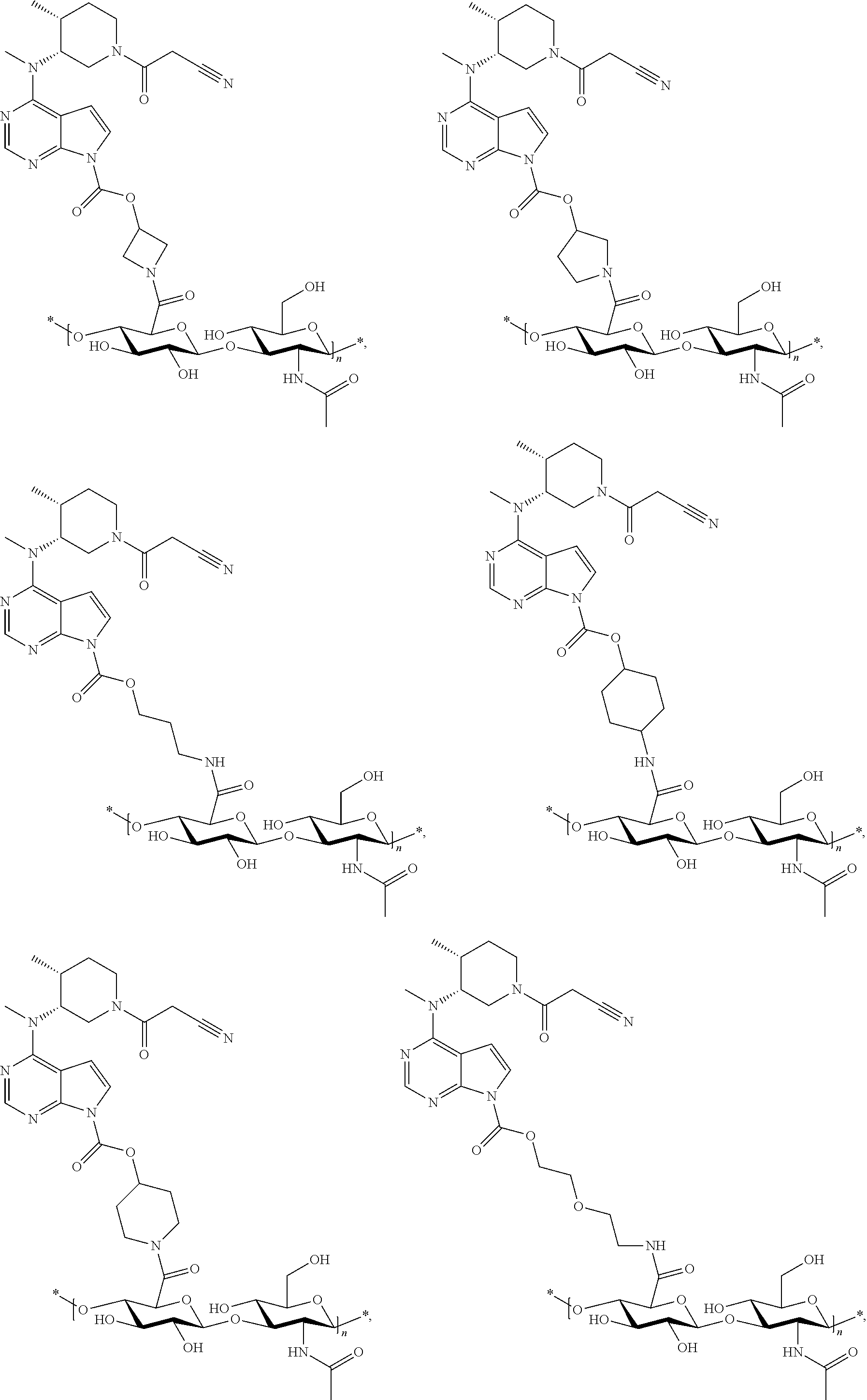

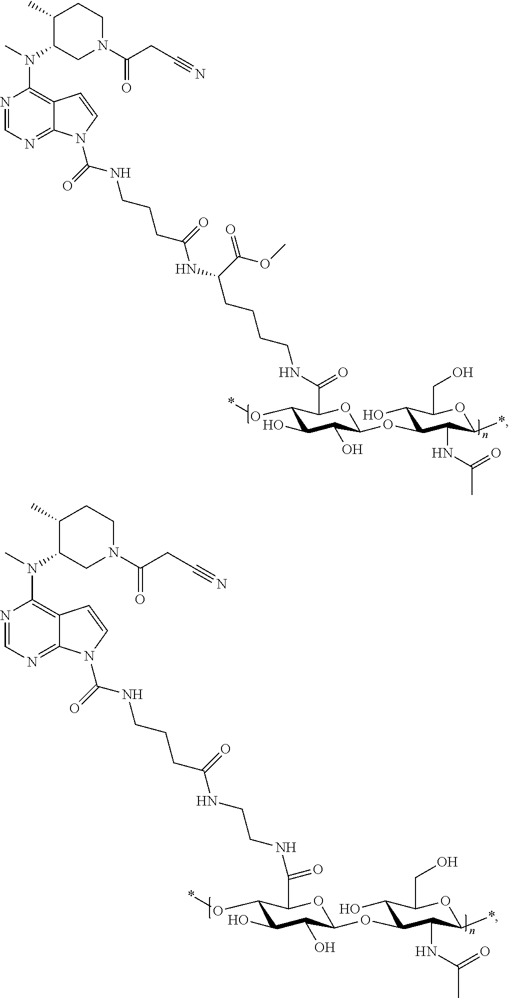

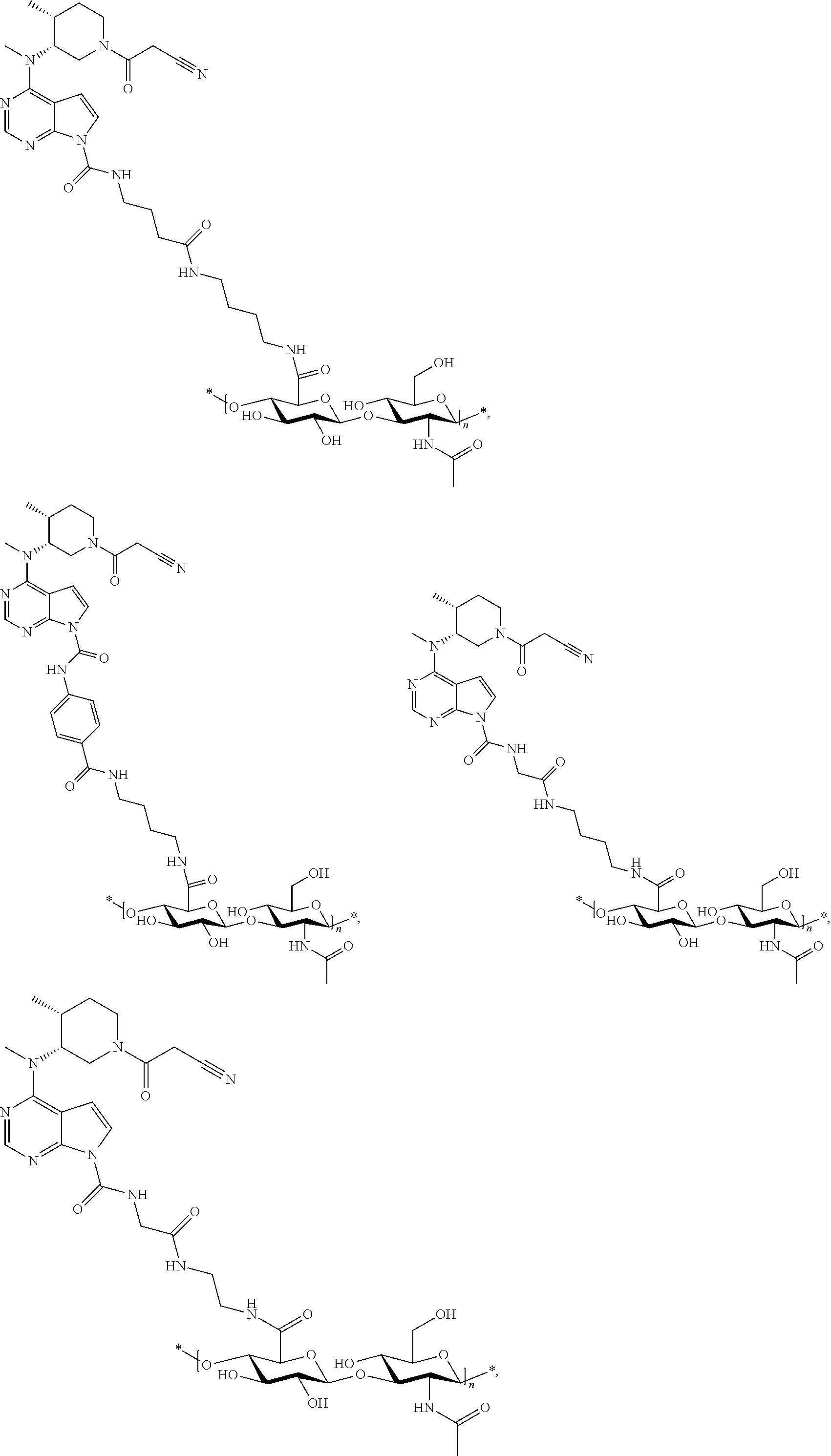

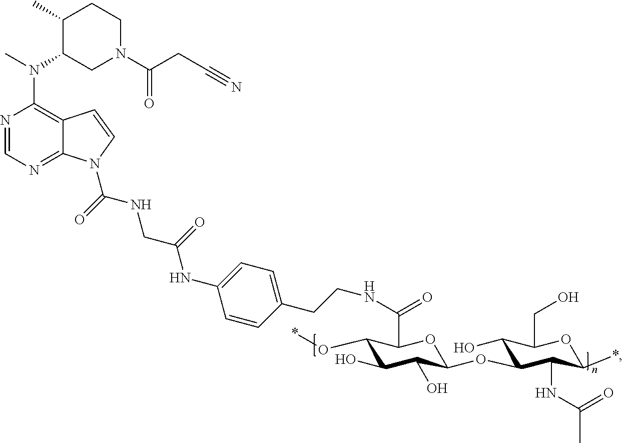

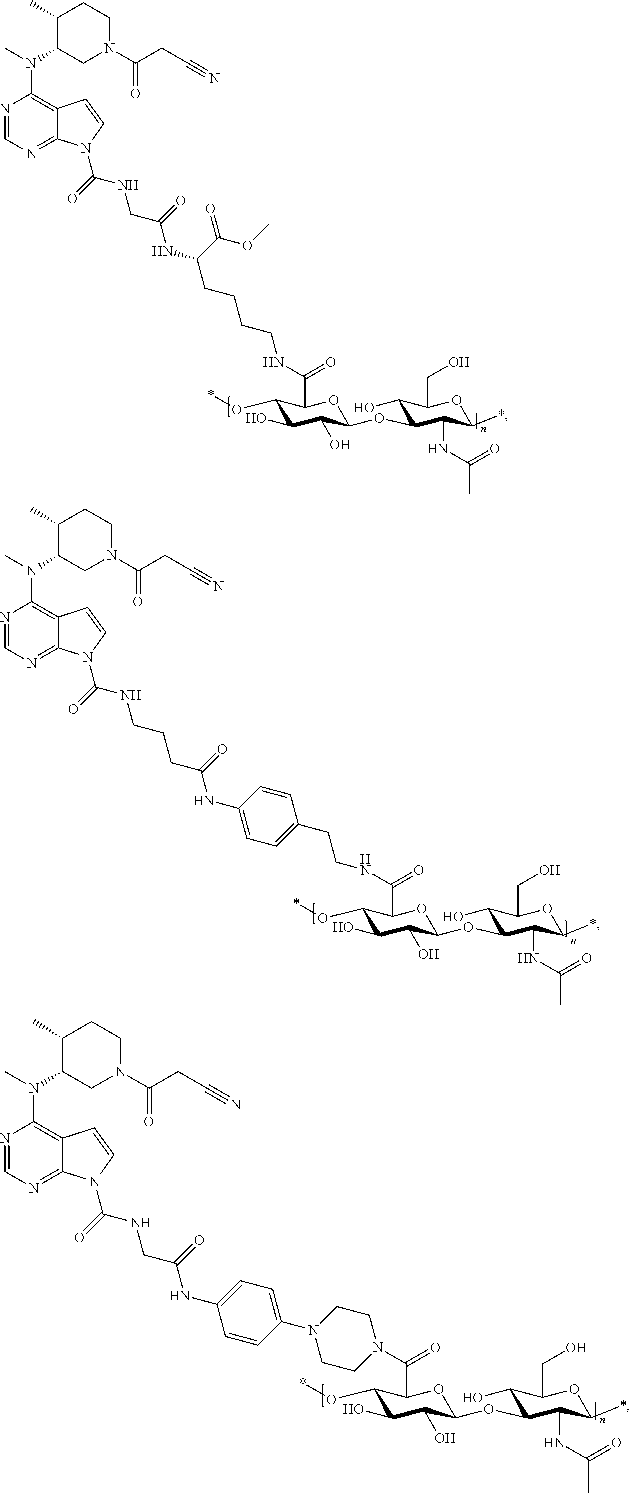

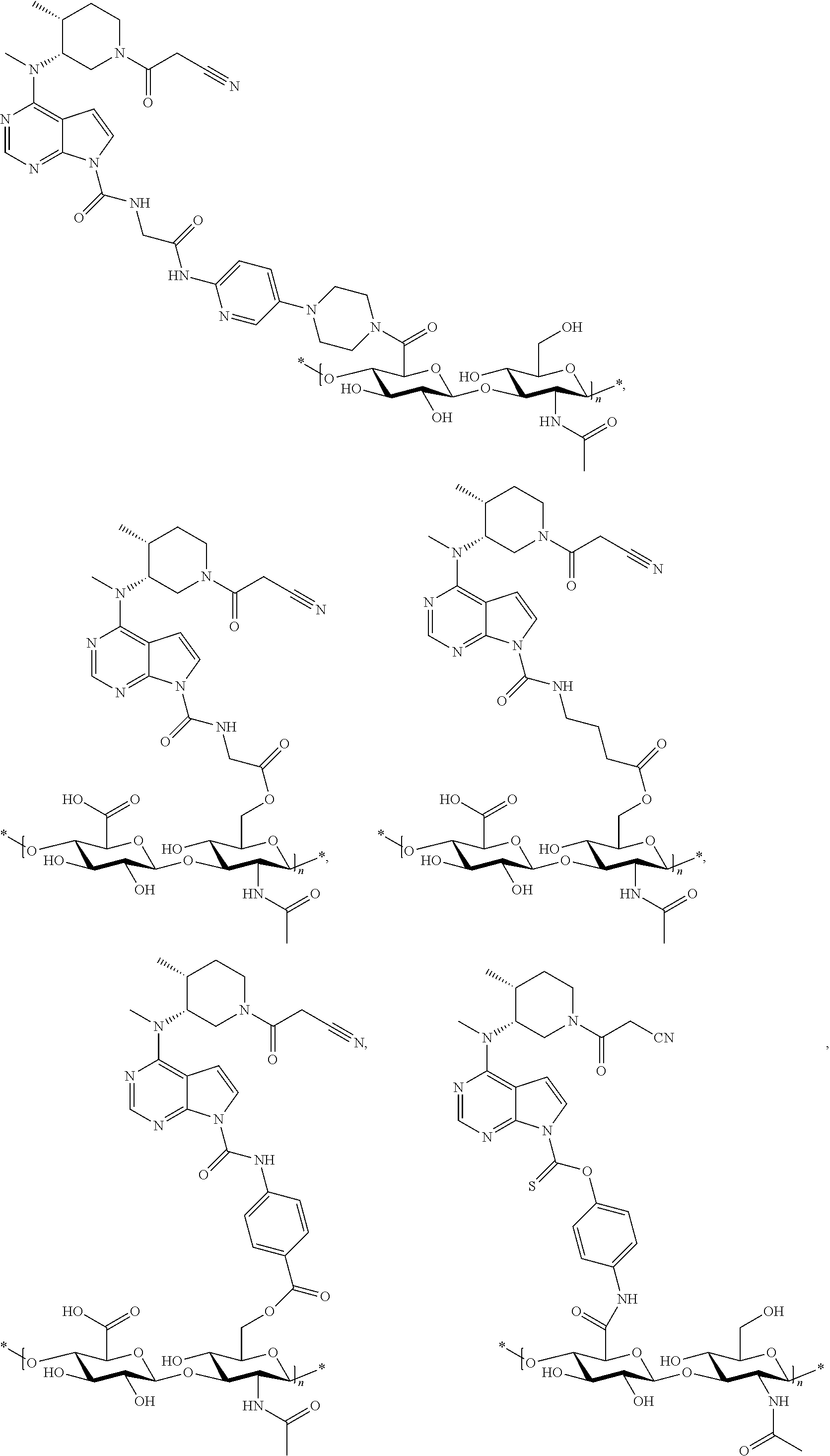

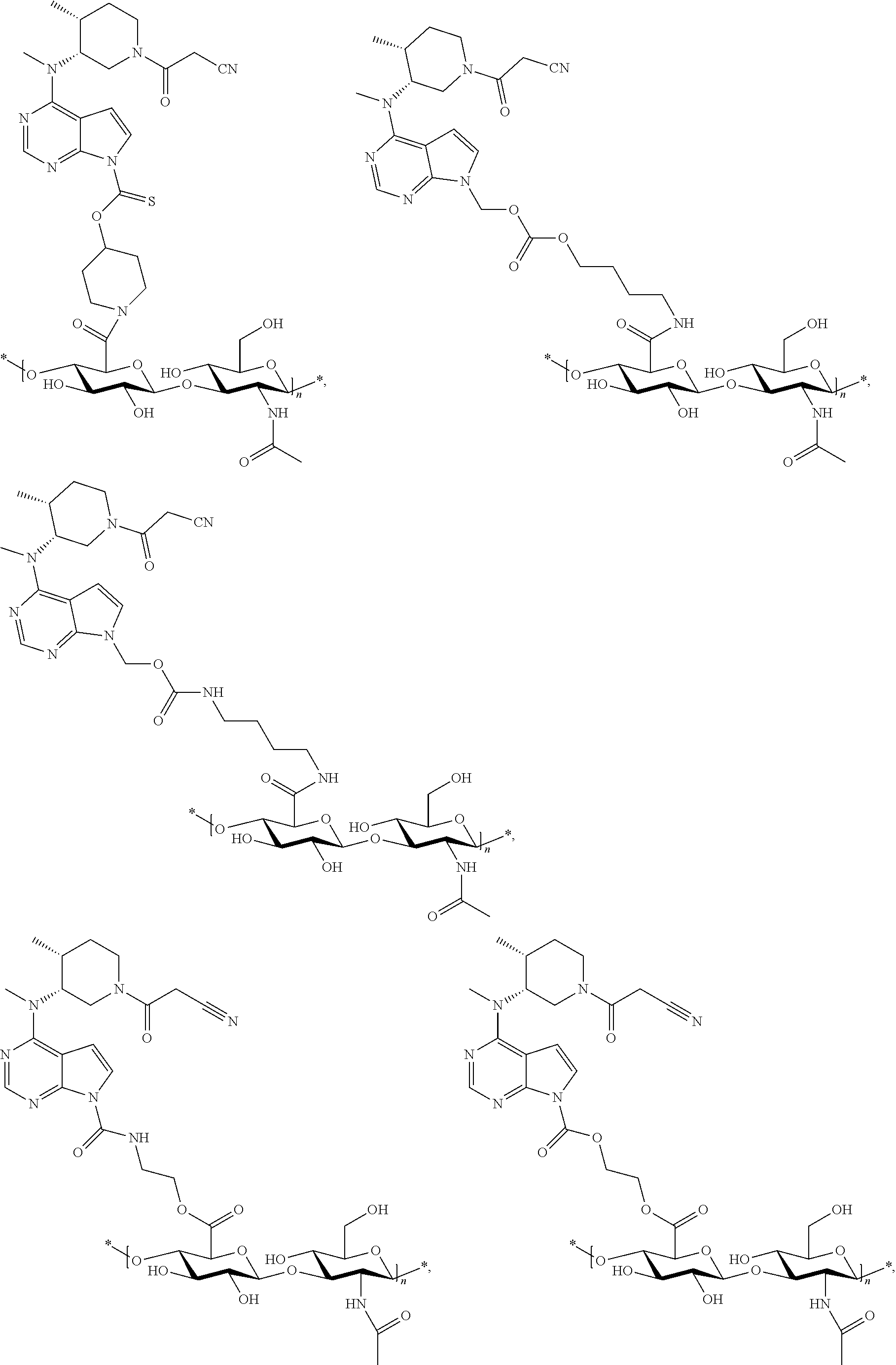

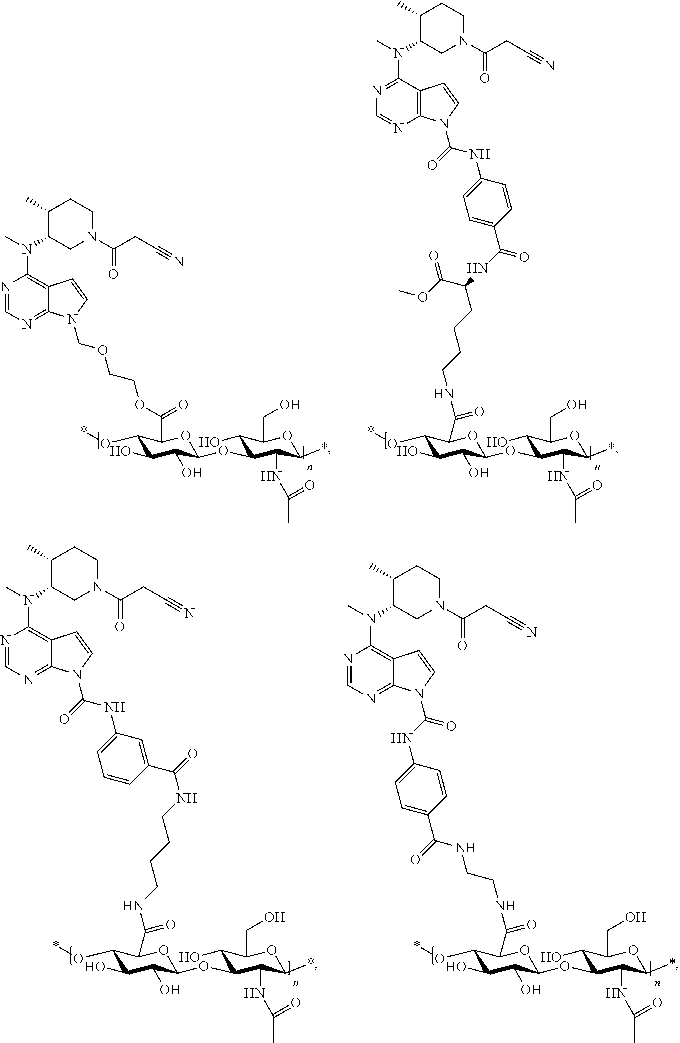

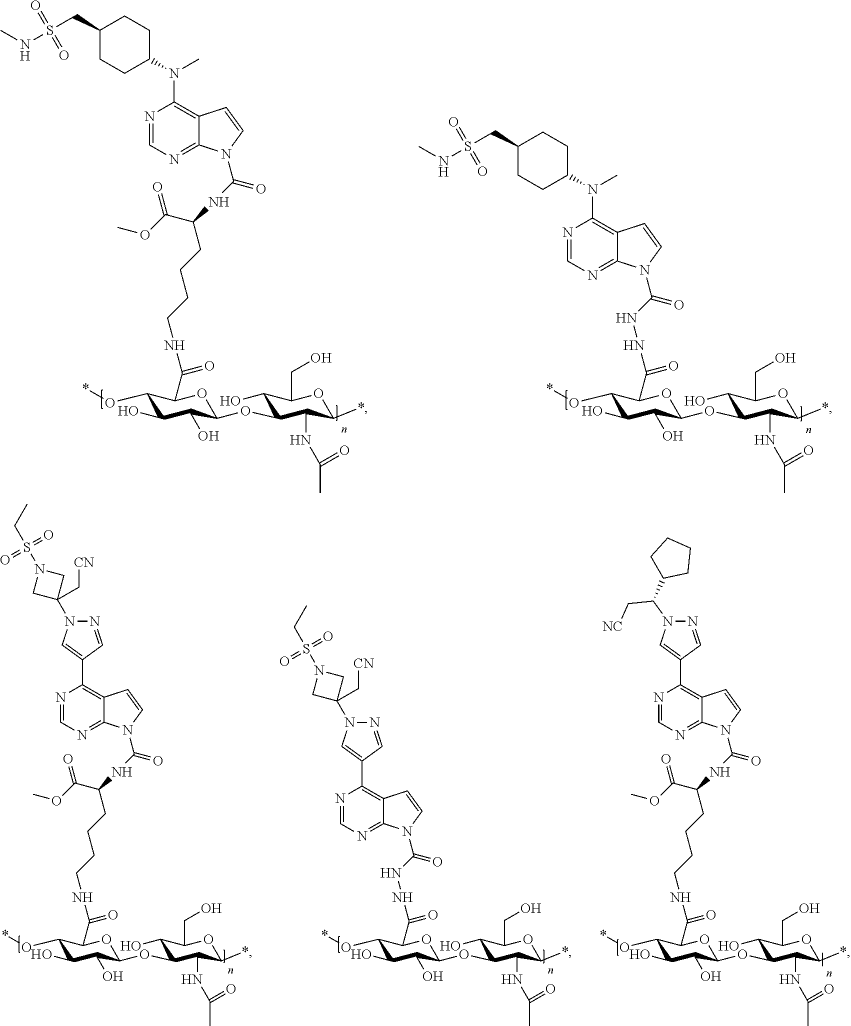

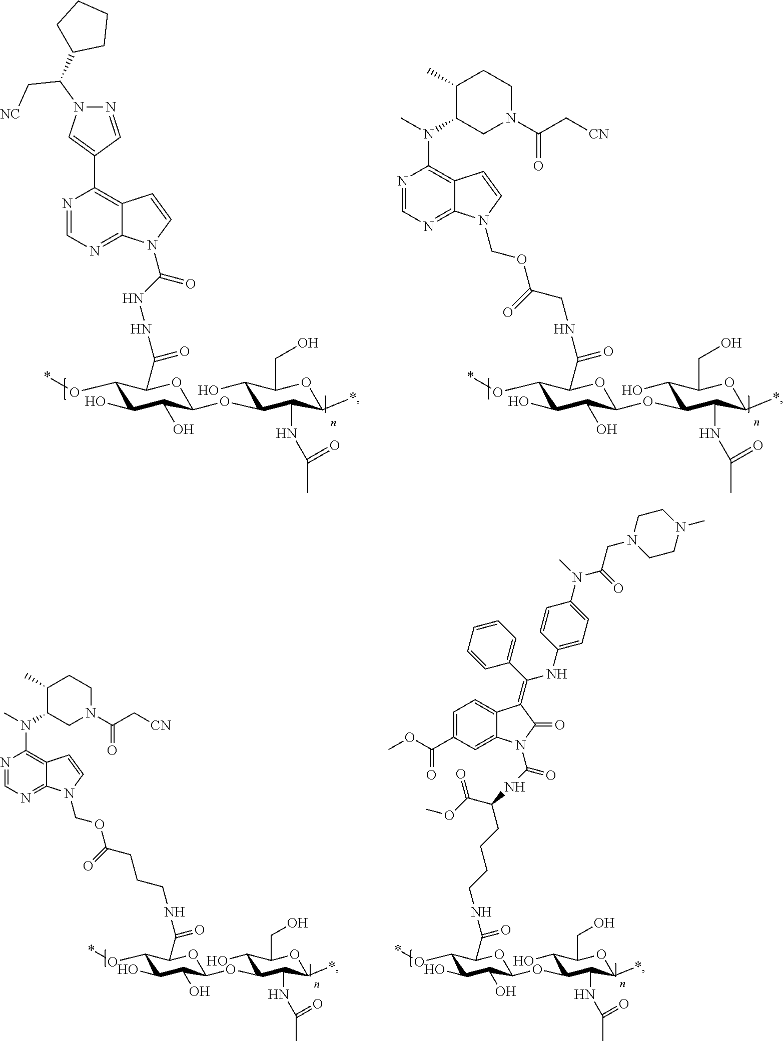

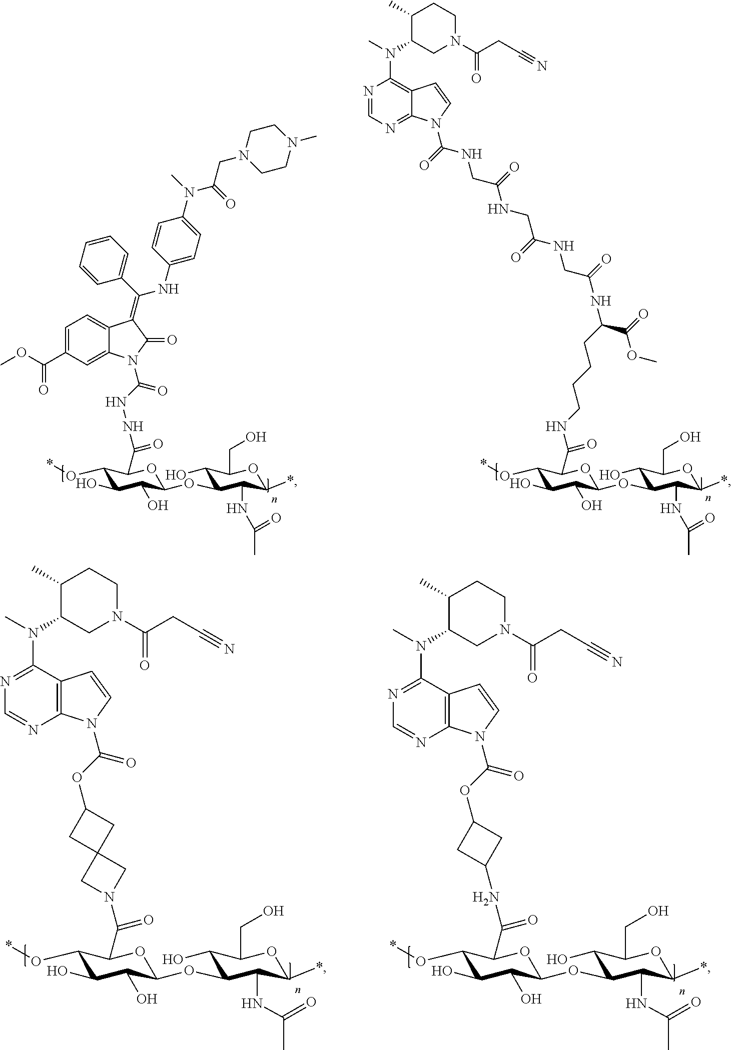

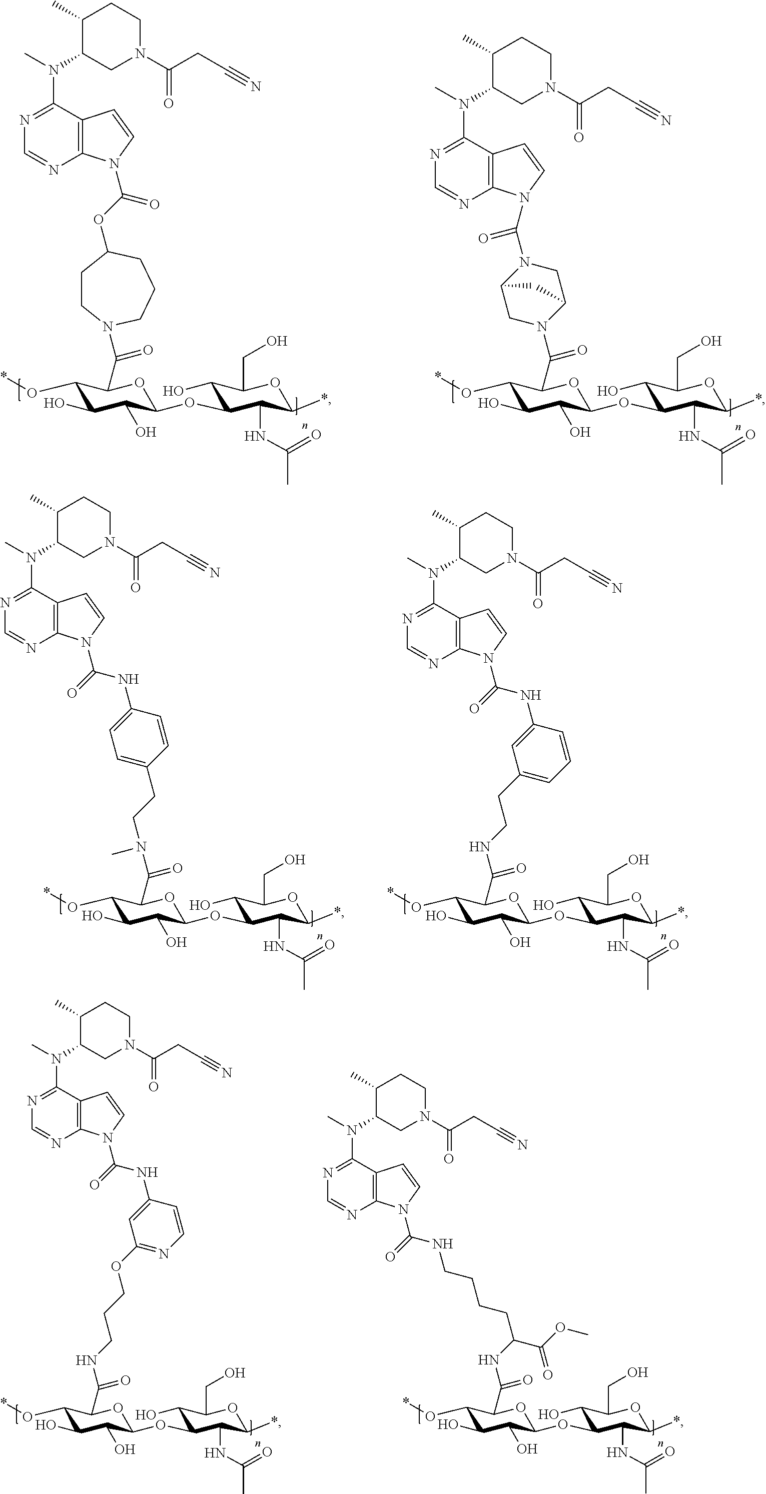

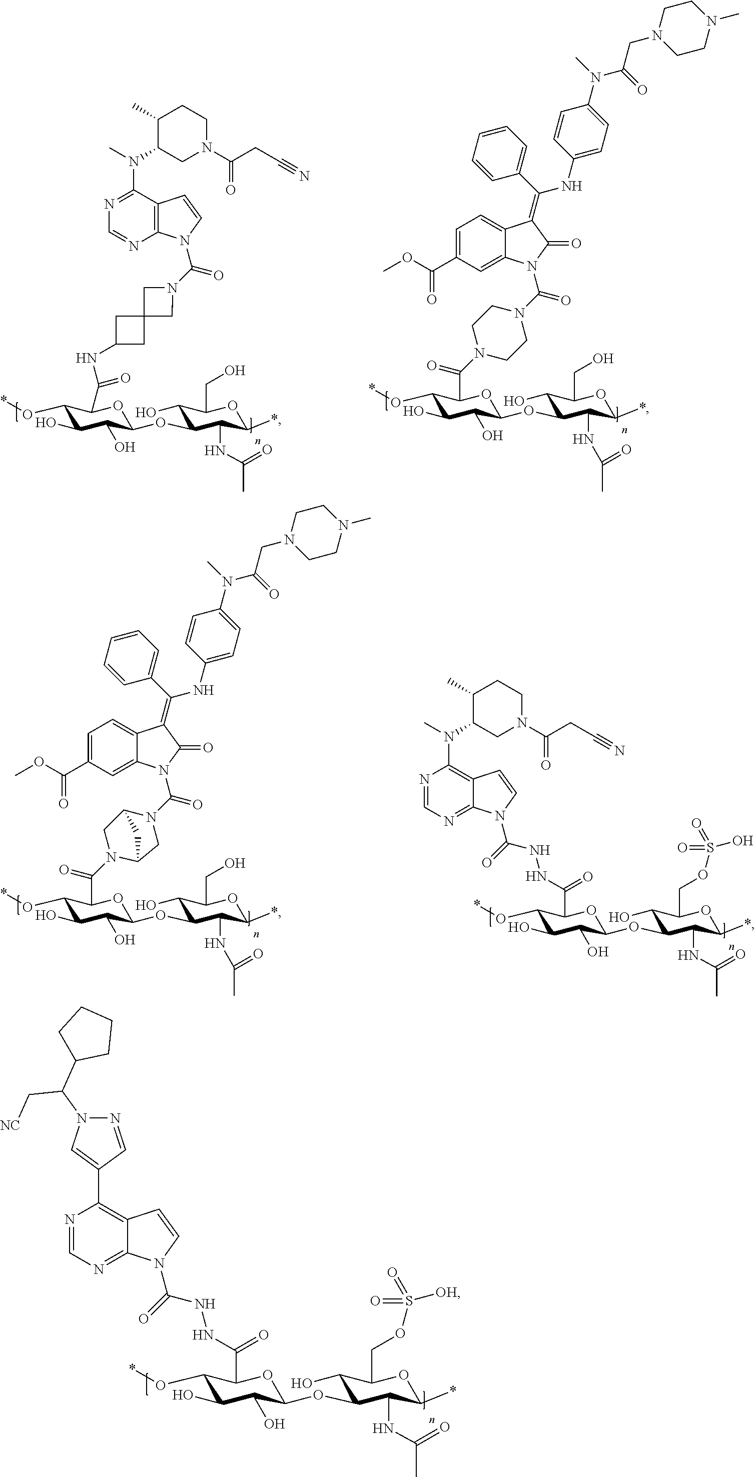

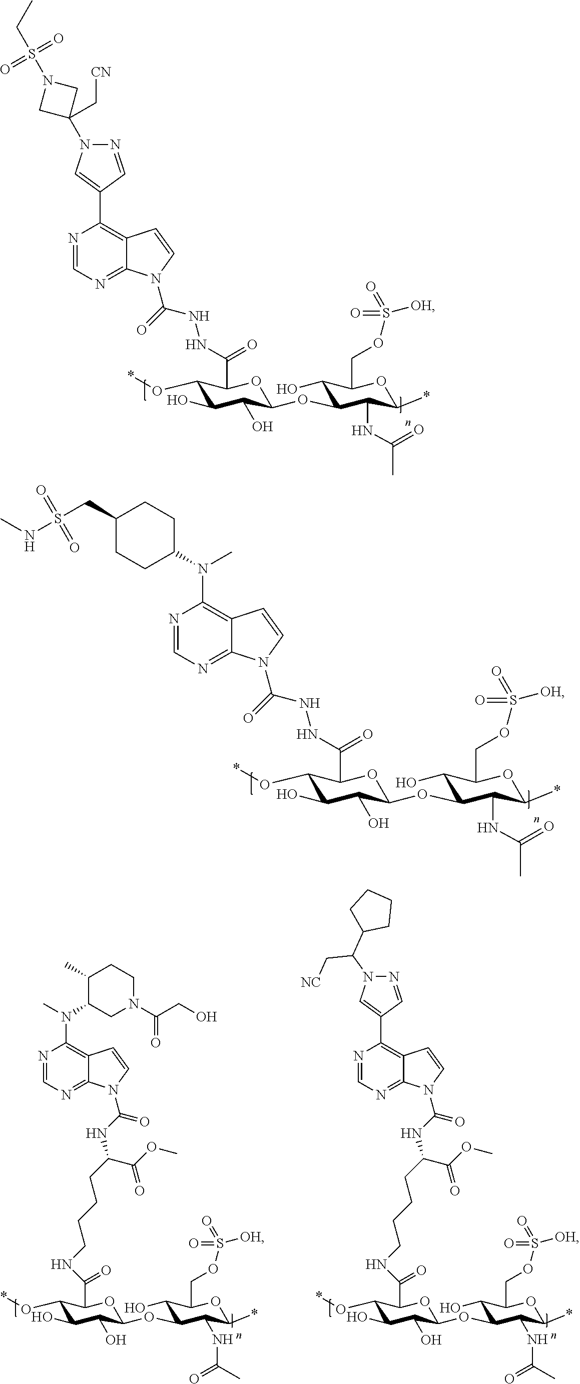

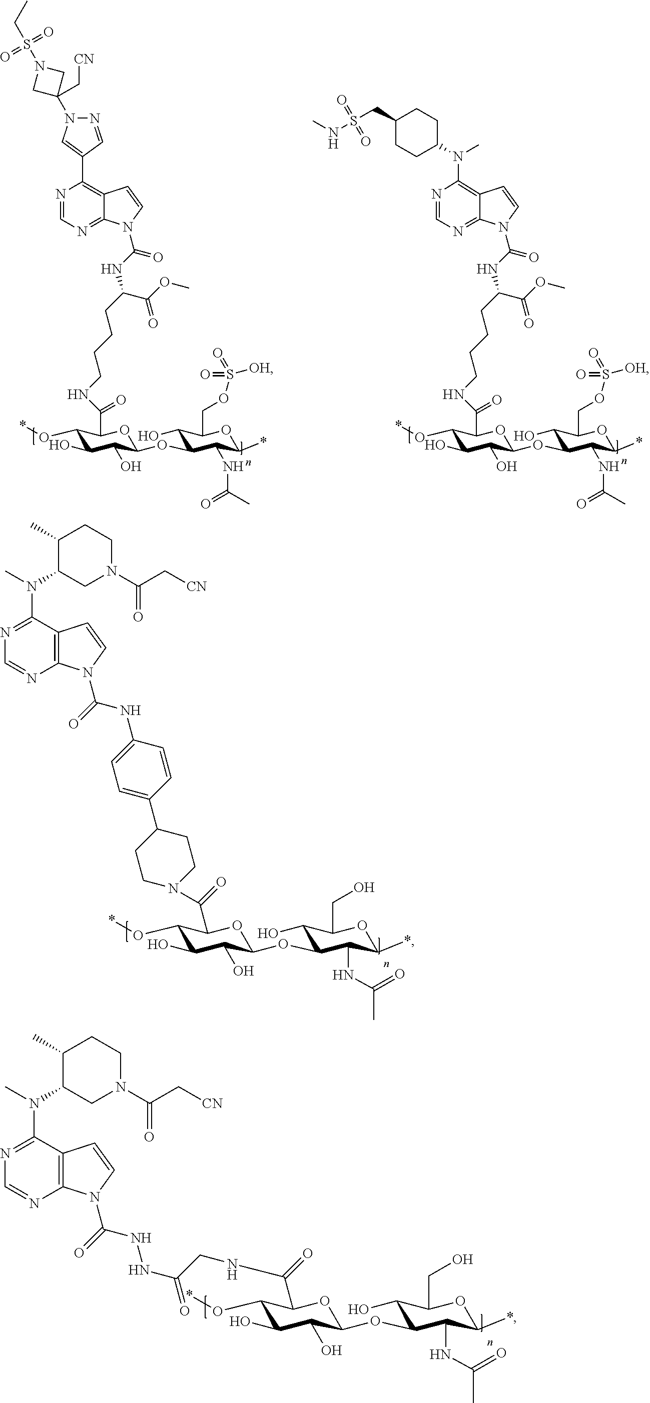

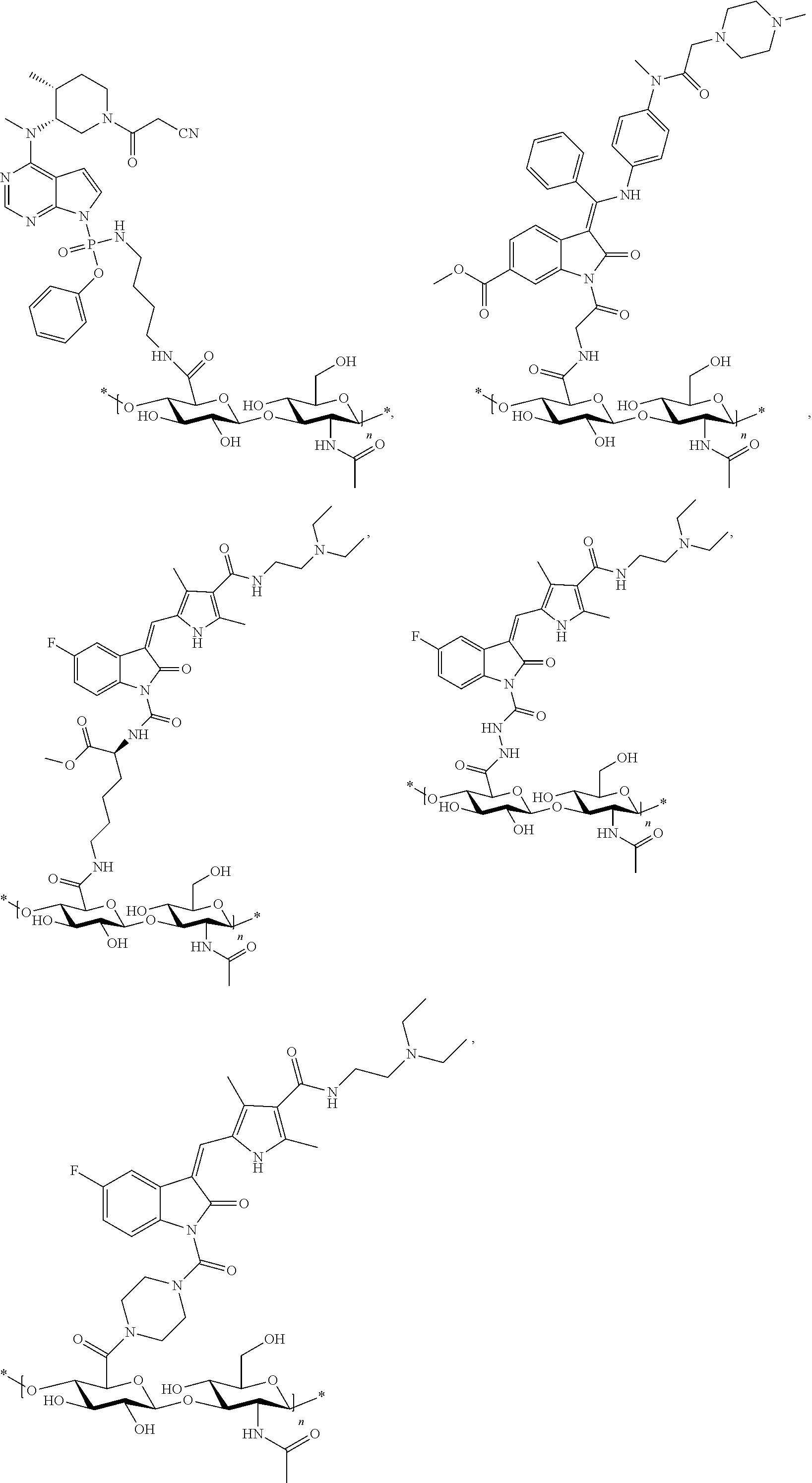

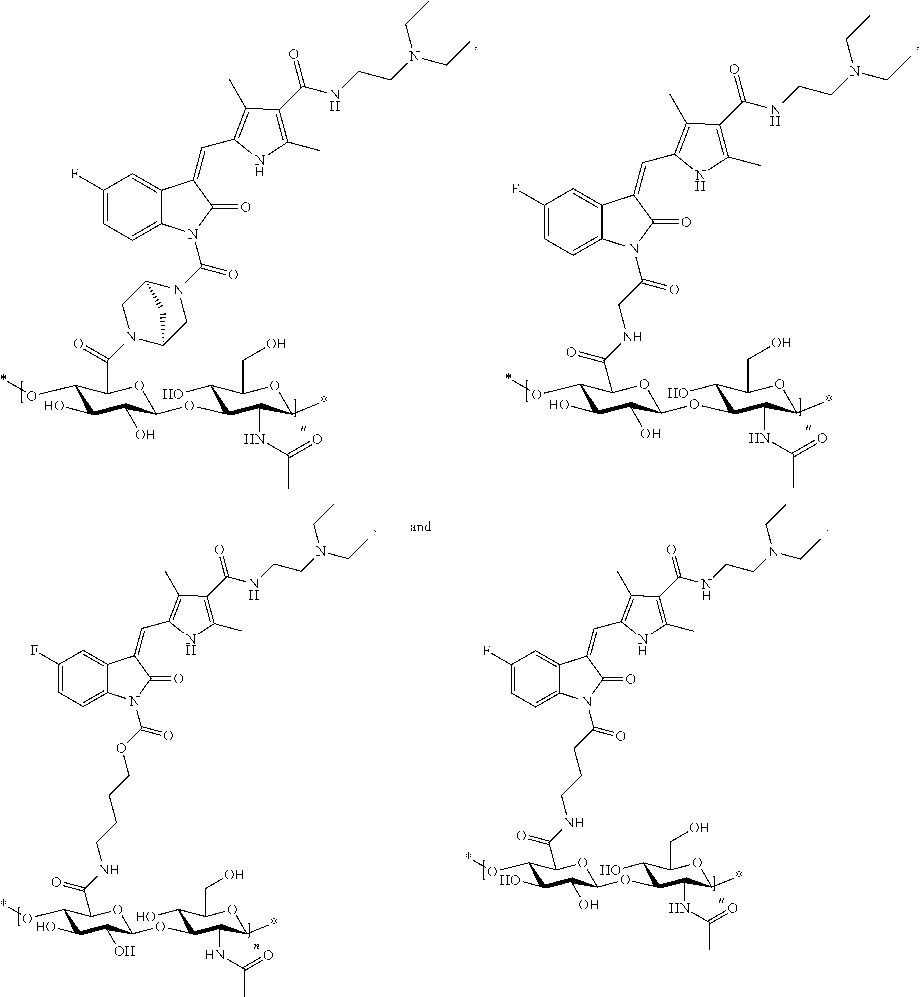

79. The drug delivery system of claim 1, wherein the drug delivery system is locally administrated to a subject in need thereof via injection, oral dosage form, inhalation, implant, or topical application.

80. A pharmaceutical composition comprising the drug delivery system according to claim 1 and a pharmaceutically acceptable excipient.

81. A method of treating a disorder in a subject in need thereof, the method comprising administering to the subject a therapeutic effective amount of the drug delivery system according to claim 1, wherein the disorder is selected from the group consisting of inflammation, cancer, cardiovascular disease, respiratory disease, disease related to vascular endothelial growth factor (VEGF), osteoarthritis, Neovascular (Wet) Age-Related Macular Degeneration (AMD), Macular Edema Following Retinal Vein Occlusion (RVO), Diabetic Macular Edema (DME), Diabetic Retinopathy (DR), Myopic Choroidal Neovascularization (mCNV), dermatitis, psoriasis, chronic obstructive pulmonary disease, and asthma.
